(19) United States
(12) Patent Application Publication
(10) Pub. No.: US 2026/0347364 A1
(43) Pub. Date: Nov. 5, 2026

(54) PYRAZOLO[4,3-F]QUINAZOLINE DERIVATIVES AS MODULATORS OF G12D MUTANT KRAS USEFUL FOR THE TREATMENT OF CANCER

(71) Applicant: JAZZ PHARMACEUTICALS IRELAND LTD., Dublin (IE)

(72) Inventors: Inder BHAMRA, Macclesfield (GB); Clifford D. JONES, Macclesfield (GB); James RYAN, Macclesfield (GB); Helen Elizabeth AYLOTT, Macclesfield (GB); Daniel J. LENG, Macclesfield (GB); Paula JACKSON, Macclesfield (GB); Kelvin HO, Macclesfield (GB)

(21) Appl. No.: 19/483,645

(22) PCT Filed: May 11, 2024
(86) PCT No.: PCT/IB2024/054597

§ 371 Date: Nov. 12, 2025

(30) Foreign Application Priority Data
May 12, 2023 (GB) 2307077.4
Dec. 20, 2023 (GB) 2319678.5

(51) Int. Cl. C07D 487/04; A61K 31/519; A61K 31/5377; A61K 31/5386; A61K 31/55; A61K 31/551; A61K 31/553; C07D 519/00

(52) U.S. Cl. CPC C07D 487/04; A61K 31/519; A61K 31/5377; A61K 31/5386; A61K 31/55; A61K 31/551; A61K 31/553; C07D 519/00

(57) ABSTRACT

This invention relates to compounds that may be useful for inhibiting RAS proteins. More specifically, this invention relates to compounds for inhibiting a broad spectrum of KRAS mutant proteins, including KRAS G12D. The compounds of the invention may therefore be used in treating conditions mediated by KRAS proteins. For example, the compounds may be used in treating cancer.

### CROSS-REFERENCE TO RELATED APPLICATIONS

The present application claims priority to GB2307077.4, filed on May 12, 2023, and entitled “COMPOUNDS,” and GB2319678.5 filed Dec. 20, 2023, and entitled “COMPOUNDS,” the entire disclosures of which are expressly incorporated by reference herein.

### FIELD OF THE INVENTION

This invention relates to compounds and their methods of use. In particular, the compounds of the present invention may be useful for inhibiting RAS proteins. More specifically, this invention relates to compounds for inhibiting a broad spectrum of KRAS proteins including mutant strains and wild-type KRAS. The compounds of the invention may therefore be used in treating conditions mediated by KRAS proteins. For example, the compounds may be used in treating cancer.

### BACKGROUND

RAS (HRAS, KRAS4A and 4B, and NRAS) proteins are a group of closely related monomeric globular proteins that act as molecular switches, cycling between inactive (GDP-bound) and active (GTP-bound) states to transduce upstream cellular signals to downstream effectors to regulate a wide variety of processes, including cellular proliferation. RAS is the most frequently mutated oncogene in cancer (~30%), with KRAS the most commonly mutated isoform accounting for ~85% of RAS mutations (Hobbs et al, Journal of Cell Science (2016) 129, 1287-1292 doi: 10.1242/jcs. 182873).

KRAS G12D is a missense gain of function mutation that results in an amino acid substitution of the glycine (G) at codon 12 with aspartic acid and is the most prevalent accounting for ~26% of all KRAS mutations in cancer. KRAS G12D mutations are present in 36% pancreatic carcinoma patients, 13% colorectal carcinoma patients, 10% rectal carcinoma patients, 6% endometrial carcinoma patients, 4% of non-small cell lung carcinoma patients, 4% gastric carcinoma patients, 3% ovarian carcinoma patients and 2% small cell lung carcinoma patients (e.g. The AACR Project GENIE Consortium, (2017) Cancer Discovery; 7 (8): 818-831. Dataset version 8). Many of these patients with G12D mutations have high unmet need with little option of efficacious targeted therapy. The mainstay of treatment for many of these patients remains chemotherapy combinations with an associated high degree of side effects and lack of efficacy.

Other KRAS missense gain of functions mutations that result in amino acid substitutions at codon 12, codon 13 and codon 61, as well as amplification of KRAS wildtype protein also drive carcinogenesis. Alterations in KRAS are found in approximately one in seven cancers (Hoffman et al, Cancer Discovery (2022) 12, 924-937). Activating mutations in KRAS are highly prevalent in solid tumours and are predominately found in 35% lung, 45% colorectal and up to 90% pancreatic cancers. G12D, G12V and G12C are the most frequently occurring KRAS mutations and are found more than half of all KRAS driven cancers. Other KRAS mutations include KRAS G12V, KRAS G12A, KRAS G13D and KRAS Q61H. KRAS amplifications are found in approximately 7% of cancers with KRAS alterations and are commonly occurring in ovarian carcinoma, breast carcinoma, lung adenocarcinoma, gastric adenocarcinoma, uterine cancers and esophagogastric cancers (Hoffman reviews). Pan KRAS inhibitors have the potential to treat a broader patient population including cancers harbouring KRAS mutations, KRAS wildtype amplifications and cancers driven by loss of the tumour suppressor NF1. In addition, pan KRAS inhibitors can potentially be used to treat cancers with acquired resistance to allele specific inhibitors such as KRAS G12C inhibitors.

Due to this frequency of KRAS mutations in multiple different tumour types and the established role of KRAS as an oncogenic driver mutation in cancer, modulating the activity of KRAS is a highly attractive therapeutic goal and has been the subject of significant research efforts for greater than 30 years. However, it has proven extremely challenging to affect KRAS activity directly and research efforts have focussed on other targets in the signalling cascade that are either upstream or downstream from KRAS. Other approaches to inhibit KRAS activity have included affecting other points on the MAPK pathway (English et al., 2002; Adjei 2014; Chin et al., 2020), many of which have shown MAPK pathway inhibition to be clinically effective. Recently, mutant KRAS G12C selective inhibitors have been reported (Kettle and Cassar 2020)., which bind covalently to an allosteric pocket and have progressed into clinical trials and shown responses in selected patients.

Compounds which are capable of modulating G12D mutant KRAS are described in WO2021/041671, WO2022/248885 and WO2022/258974. Compounds capable of modulating multiple RAS isoforms and mutants have also been described (Kessler et al. 2019), however these compounds are believed to be of limited therapeutic benefit owing to a lack of sufficient potency as well as little selectivity for KRAS over HRAS and NRAS isoforms.

An aim of the present invention is to provide alternative or improved compounds for inhibiting RAS proteins. For example, an aim of the present invention is to provide alternative or improved compounds for inhibiting KRAS proteins.

Furthermore, it is an aim of certain embodiments of this invention to provide new compounds for use in treatment of conditions modulated by RAS proteins. For example, it is an aim of certain embodiments of this invention to provide compounds for use in the treatment of cancer. Said compounds may be more selective for KRAS proteins having the G12D mutation over alternative KRAS proteins than prior art compounds. Alternatively, said compounds may have broad spectrum activity across a range of KRAS proteins.

It is an aim of certain embodiments of this invention to provide new cancer treatments. In particular, it is an aim of certain embodiments of this invention to provide compounds which have comparable activity to existing treatments, optionally they should have better activity.

It is an aim of certain embodiments of this invention to provide compounds which exhibit reduced cytotoxicity relative to prior art compounds and existing therapies.

Another aim of certain embodiments of this invention is to provide compounds having a convenient pharmacokinetic profile and a suitable duration of action following dosing. A further aim of certain embodiments of this invention is to provide compounds in which the metabolised fragment or fragments of the drug after absorption are GRAS (Generally Regarded As Safe).

Certain embodiments of the present invention satisfy some or all of the above aims.

### BRIEF SUMMARY OF THE DISCLOSURE

In accordance with the present inventions there is provided a compound of formula (Ia), or a pharmaceutically acceptable salt thereof:

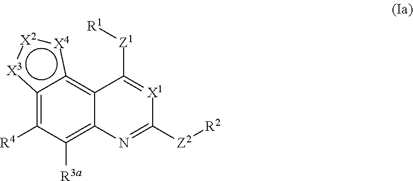

wherein
Z¹ is independently selected from —O— and —NR^{5a}—;
Z² is independently absent or is selected from —O— and —NR^{5b}—;
X¹ is independently selected from N and CR^{3b};
X² is selected from N, NR⁶, O and S;
X³ is selected from N and NR⁶;
X⁴ is CR^{3c};
R¹ is independently selected from C₀-C₃-alkylene-R^{1a} and C₂-C₆-alkylene-R^{1b}; wherein R^{1a} is independently selected from: a 4- to 7-membered heterocycloalkyl ring; a phenyl ring; and a C₃-C₇-cycloalkyl ring optionally substituted with an NR⁷R⁸ group; wherein said heterocycloalkyl ring, said phenyl ring or said cycloalkyl ring is optionally substituted with from 1 to 4 R⁹ groups;
R^{1b} is independently selected from: NR⁷R⁸, OR⁸, SR⁸, SOR⁸, SO²R⁸ and SO(NH)R⁸;
or R¹ and R^{5a} together with the nitrogen to which they are attached form a ring system selected from: a monocyclic 4- to 7-membered heterocycloalkyl group, optionally substituted with from 1 to 4 R⁹ groups; and a fused, spirofused or bridged bicyclic 6- to 11-membered heterocyclyl group, optionally substituted with from 1 to 4 R⁹ groups;
R² is independently C₁-C₆-alkyl, C₁-C₄-haloalkyl, C₀-C₄-alkylene-R^{2a}, C₁-C₄-alkylene-R^{2b}, C₂-C₄-alkylene-R^{2c};
R^{2a} is independently selected from monocyclic 4- to 7-membered heterocycloalkyl group; a fused, spirofused or bridged bicyclic 6- to 11-membered heterocycloalkyl group; a 5-, 6-, 9- or 10-membered monocyclic or bicyclic heteroaryl group; phenyl; and C₃-C₇-cycloalkyl; wherein any heterocycloalkyl or cycloalkyl R^{2a} group is optionally substituted with from 1 to 6 R¹⁰ groups and any heteroaryl or phenyl R^{2a} group is optionally substituted with from 1 to 6 R¹¹ groups;
wherein R^{2b} is independently selected from CONR¹²R¹² and CO₂R¹²;
wherein R^{2c} is independently selected from NR¹²R¹³ and OR¹²;
or R² and R^{5b} together with the nitrogen to which they are attached form a ring system selected from: monocyclic 4- to 7-membered heterocycloalkyl group; and a fused, spirofused or bridged bicyclic 6- to 11-membered heterocycloalkyl group, said heterocycloalkyl group being optionally substituted with from 1 to 6 R¹⁰ groups;
R^{3a}, R^{3b} and R^{3c} are each independently selected from: H, halo, C₁-C₄-alkyl, O—C₁-C₄-alkyl, C₁-C₄-haloalkyl, O—C₁-C₄-haloalkyl, cyclopropyl, nitro and cyano;
R⁴ is independently selected from phenyl, said phenyl being optionally fused to a C₅-C₇-cycloalkyl ring; naphthyl; monocyclic 4- to 7-membered cycloalkyl or heterocycloalkyl; and 5-, to 10-membered monocyclic or bicyclic heterocyclyl; wherein R⁴ is optionally substituted with from 1 to 4 R¹⁴ groups;
R^{5a}, R^{5b}, R⁸ and R¹² are each independently selected at each occurrence from H, C₁-C₄-haloalkyl, cyclopropyl and C₁-C₄-alkyl;
R⁶ is independently selected from H, C₁-C₄-haloalkyl, C₁-C₄-alkyl and C₃-C₄-cycloalkyl;
R⁷ and R¹³ are each independently at each occurrence selected from H, C₁-C₄-alkyl, C₁-C₄-haloalkyl and C(O)—C₁-C₄-alkyl;

or R¹² and R¹³ together with the nitrogen to which they are attached form a ring system selected from: monocyclic 4- to 7-membered heterocycloalkyl group; and a fused, spirofused or bridged bicyclic 6- to 11-membered heterocycloalkyl group; said heterocycloalkyl group being optionally substituted with from 1 to 6 R^{10a} groups;
R⁹ is independently at each occurrence selected from oxo, halo, cyano, NR¹²R¹³, OR¹², COR¹², CO₂R¹², CONR¹²R¹², CONR¹²R¹³, C₁-C₄-alkyl, C₁-C₄-alkyl substituted with NR¹²R¹³, C₁-C₄-alkyl substituted with OR¹², C₁-C₄-alkyl substituted with cyano, C₂-C₄-alkenyl, C₂-C₄-alkynyl, C₁-C₄-haloalkyl and cyclopropyl;
R¹⁰ is independently at each occurrence selected from oxo, halo, cyano, NR¹²R¹³, OR¹², COR¹², CO₂R¹², CONR¹²R¹², C₁-C₄-alkyl, C₁-C₄-alkyl substituted with NR¹²R¹³, C₁-C₄-alkyl substituted with OR¹², C₁-C₄-alkyl substituted with cyano, C₁-C₄-alkyl substituted with phenyl, C₂-C₄-alkenyl, C₂-C₄-alkynyl, C₁-C₄-haloalkyl and cyclopropyl;
R^{10a} is independently at each occurrence selected from oxo, halo, cyano, NR¹²R^{13a}, OR¹², COR¹², CO₂R¹², CONR¹²R¹², CONR¹²R^{13a}, C₁-C₄-alkyl, C₁-C₄-alkyl substituted with NR¹²R^{13a} C₁-C₄-alkyl substituted with OR¹², C₁-C₄-alkyl substituted with cyano, C₁-C₄-alkyl substituted with phenyl, C₂-C₄-alkenyl, C₂-C₄-alkynyl, C₁-C₄-haloalkyl and cyclopropyl;
R^{13a} is independently at each occurrence selected from H, C₁-C₄-alkyl, C₁-C₄-haloalkyl and C(O)—C₁-C₄-alkyl;
R¹¹ is independently selected from halo, cyano, nitro, NR¹²R¹³, OR¹², CO₂R¹², CONR¹²R¹², C₁-C₄-alkyl, C₁-C₄-alkyl substituted with NR¹²R¹³, C₁-C₄-alkyl substituted with OR¹², monocyclic 4- to 7-membered cycloalkyl or heterocycloalkyl, C₂-C₄-alkenyl, C₂-C₄-alkynyl, C₁-C₄-haloalkyl and cyclopropyl;
R¹⁴ is independently at each occurrence selected from halo, cyano, nitro, NR¹²R¹³, OR¹², CO₂R¹², CONR¹²R¹², C₁-C₄-alkyl, C₁-C₄-alkyl substituted with NR¹²R¹³, C₁-C₄-alkyl substituted with OR¹², C₂-C₄-alkenyl, C₂-C₄-alkynyl, C₁-C₄-haloalkyl, phenyl and cyclopropyl;
wherein any of the aforementioned alkyl, alkylene, phenyl or cycloalkyl (e.g. cyclopropyl) groups is optionally substituted, where chemically possible, by 1 to 5 substituents which are each independently at each occurrence selected from the group consisting of: C₁-C₄-alkyl, C₁-C₄-alkyl substituted with OR^{a}, halo, nitro, cyano, NR^{a}R^{b}, OR^{a}, SR^{a}, CO₂R^{a}, C(O)R^{a}, CONR^{a}R^{a};
wherein R^{a} is independently at each occurrence selected from H, C₁-C₄-alkyl and C₁-C₄-haloalkyl; and R^{b} is independently at each occurrence selected from H, C₁-C₄-alkyl, C(O)—C₁-C₄-alkyl and S(O)₂—C₁-C₄-alkyl.

In accordance with the present inventions there is provided a compound of formula (I), or a pharmaceutically acceptable salt thereof:

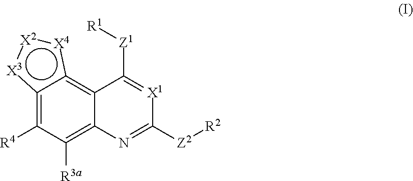

Z¹ is independently selected from —O— and —NR^{5a}—;
Z² is independently absent or is selected from —O— and —NR^{5b}—;
X¹ is independently selected from N and CR^{3b};
X² is selected from N, NR⁶, O and S;
X³ is selected from N and NR⁶;
X⁴ is CR^{3c};
R¹ is independently selected from C₀-C₃-alkylene-R^{1a} and C₂-C₆-alkylene-R^{1b}; wherein R^{1a} is independently selected from an oxygen containing 4- to 7-membered heterocycloalkyl ring, a 4- to 7-membered heterocycloalkyl ring; and a C₃-C₇-cycloalkyl ring substituted with an NR⁷R⁸ group; wherein said heterocycloalkyl ring or said cycloalkyl ring is optionally substituted with from 1 to 4 R⁹ groups; R^{1b} is independently selected from: NR⁷R⁸, OR⁸, SR⁸, SOR⁸, SO₂R⁸ and SO(NH)R⁸;
or R¹ and R^{5a} together with the nitrogen to which they are attached form a ring system selected from: a monocyclic 4- to 7-membered heterocycloalkyl group, optionally substituted with from 1 to 4 R⁹ groups; a fused, spirofused or bridged bicyclic 6- to 11-membered heterocyclyl group, optionally substituted with from 1 to 4 R⁹ groups;
R² is independently C₁-C₆-alkyl, C₁-C₄-haloalkyl, C₀-C₄-alkylene-R^{2a}, C₁-C₄-alkylene-R^{2b}, C₂-C₄-alkylene-R^{2c};
R^{2a} is independently selected from monocyclic 4- to 7-membered heterocycloalkyl group, a fused, spirofused or bridged bicyclic 6- to 11-membered heterocycloalkyl group; a 5-, 6-, 9- or 10-membered monocyclic or bicyclic heteroaryl group; phenyl; C₃-C₇-cycloalkyl; wherein any heterocycloalkyl or cycloalkyl R^{2a} group is optionally substituted with from 1 to 6 R¹⁰ groups and any heteroaryl or phenyl R^{2a} group is optionally substituted with from 1 to 6 R¹¹ groups;
wherein R^{2b} is independently selected from CONR¹²R¹² and CO₂R¹²;
wherein R^{2c} is independently selected from NR¹²R¹³ and OR¹²;
or R² and R^{5b} together with the nitrogen to which they are attached form a ring system selected from: monocyclic 4- to 7-membered heterocycloalkyl group, a fused, spirofused or bridged bicyclic 6- to 11-membered heterocycloalkyl group, said heterocycloalkyl group being optionally substituted with from 1 to 6 R¹⁰ groups;
R^{3a}, R^{3b} and R^{3c} are each independently selected from: H, halo, C₁-C₄-alkyl, O—C₁-C₄-alkyl, C₁-C₄-haloalkyl, O—C₁-C₄-haloalkyl, cyclopropyl, nitro and cyano;
R⁴ is independently selected from phenyl, said phenyl being optionally fused to a C₅-C₇-cycloalkyl ring; naphthyl; monocyclic 4- to 7-membered cycloalkyl or heterocycloalkyl; and 5-, to 10-membered monocyclic or bicyclic heterocyclyl, wherein R⁴ is optionally substituted with from 1 to 4 R¹⁴ groups;
R^{5a}, R^{5b}, R⁸ and R¹² are each independently selected at each occurrence from H, C₁-C₄-haloalkyl, and C₁-C₄-alkyl;
R⁶ is independently selected from H, C₁-C₄-haloalkyl, C₁-C₄-alkyl and C₃-C₄-cycloalkyl;
R⁷ and R¹³ are each independently at each occurrence selected from H, C₁-C₄-alkyl, C₁-C₄-haloalkyl and C(O)—C₁-C₄-alkyl;
or R¹² and R¹³ together with the nitrogen to which they are attached form a ring system selected from: monocyclic 4- to 7-membered heterocycloalkyl group, a fused, spirofused or bridged bicyclic 6- to 11-membered heterocycloalkyl group, said heterocycloalkyl group being optionally substituted with from 1 to 6 R¹⁰ groups;
R⁹ and R¹⁰ are each independently at each occurrence selected from oxo, halo, cyano, NR¹²R¹³, OR¹², CO₂R¹², CONR¹²R¹², C₁-C₄-alkyl, C₁-C₄-alkyl substituted with NR¹²R¹³, C₁-C₄-alkyl substituted with OR¹², C₁-C₄-alkyl substituted with cyano, C₂-C₄-alkenyl, C₂-C₄-alkynyl, C₁-C₄-haloalkyl and cyclopropyl;
R¹¹ is independently selected from halo, cyano, nitro, NR¹²R¹³, OR¹², CO₂R¹², CONR¹²R¹², C₁-C₄-alkyl, C₁-C₄-alkyl substituted with NR¹²R¹³, C₁-C₄-alkyl substituted with OR¹², monocyclic 4- to 7-membered cycloalkyl or heterocycloalkyl, C₂-C₄-alkenyl, C₂-C₄-alkynyl, C₁-C₄-haloalkyl and cyclopropyl;
R¹⁴ is independently at each occurrence selected from halo, cyano, nitro, NR¹²R¹³, OR¹², CO₂R¹², CONR¹²R¹², C₁-C₄-alkyl, C₁-C₄-alkyl substituted with NR¹²R¹³, C₁-C₄-alkyl substituted with OR¹², C₂-C₄-alkenyl, C₂-C₄-alkynyl, C₁-C₄-haloalkyl and cyclopropyl;
wherein any of the aforementioned alkyl, alkylene or cycloalkyl (e.g. cyclopropyl) groups is optionally substituted, where chemically possible, by 1 to 5 substituents which are each independently at each occurrence selected from the group consisting of: C₁-C₄-alkyl, halo, nitro, cyano, NR^{a}R^{b}, OR^{a}, SR^{a}, CO₂R^{a}, C(O)R^{a}, CONR^{a}R^{a}; wherein R^{a} is independently at each occurrence selected from H, C₁-C₄-alkyl and C₁-C₄-haloalkyl; and R^{b} is independently at each occurrence selected from H, C₁-C₄-alkyl, C(O)—C₁-C₄-alkyl and S(O)₂—C₁-C₄-alkyl.

In accordance with the present inventions there is provided a compound of formula (Ib), or a pharmaceutically acceptable salt thereof:

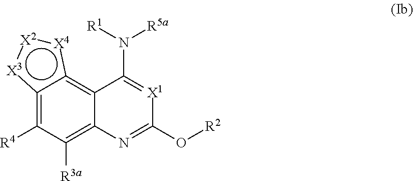

wherein
X¹ is independently selected from N and CR^{3b};
X² is selected from N, NR⁶, O and S;
X³ is selected from N and NR⁶;
X⁴ is CR^{3c};
R¹ is independently selected from C₀-C₃-alkylene-R^{1a} and C₂-C₆-alkylene-R^{1b}; wherein R^{1a} is independently selected from; a 4- to 7-membered heterocycloalkyl ring; a phenyl ring; and a C₃-C₇-cycloalkyl ring optionally substituted with an NR⁷R⁸ group; wherein said heterocycloalkyl ring, said phenyl ring or said cycloalkyl ring is optionally substituted with from 1 to 4 R⁹ groups;
R^{1b} is independently selected from: NR⁷R⁸, OR⁸, SR⁸, SOR⁸, SO₂R⁸ and SO(NH)R⁸;
or R¹ and R^{5a} together with the nitrogen to which they are attached form a ring system selected from: a monocyclic 4- to 7-membered heterocycloalkyl group, optionally substituted with from 1 to 4 R⁹ groups; and a fused, spirofused or bridged bicyclic 6- to 11-membered heterocyclyl group, optionally substituted with from 1 to 4 R⁹ groups;
R² is independently C₁-C₆-alkyl, C₁-C₄-haloalkyl, C₀-C₄-alkylene-R^{2a}, C₁-C₄-alkylene-R^{2b}, C₂-C₄-alkylene-R^{2c};
R^{2a} is independently selected from monocyclic 4- to 7-membered heterocycloalkyl group; a fused, spirofused or bridged bicyclic 6- to 11-membered heterocycloalkyl group; a 5-, 6-, 9- or 10-membered monocyclic or bicyclic heteroaryl group; phenyl; C₃-C₇-cycloalkyl; wherein any heterocycloalkyl or cycloalkyl R^{2a} group is optionally substituted with from 1 to 6 R¹⁰ groups and any heteroaryl or phenyl R^{2a} group is optionally substituted with from 1 to 6 R¹¹ groups;
wherein R^{2b} is independently selected from CONR¹²R¹² and CO₂R¹²;
wherein R^{2c} is independently selected from NR¹²R¹³ and OR¹²;
or R² and R^{5b} together with the nitrogen to which they are attached form a ring system selected from: monocyclic 4- to 7-membered heterocycloalkyl group; and a fused, spirofused or bridged bicyclic 6- to 11-membered heterocycloalkyl group, said heterocycloalkyl group being optionally substituted with from 1 to 6 R¹⁰ groups;
R^{3a}, R^{3b} and R^{3c} are each independently selected from: H, halo, C₁-C₄-alkyl, O—C₁-C₄-alkyl, C₁-C₄-haloalkyl, O—C₁-C₄-haloalkyl, cyclopropyl, nitro and cyano;
R⁴ is independently selected from phenyl, said phenyl being optionally fused to a C₅-C₇-cycloalkyl ring; naphthyl; monocyclic 4- to 7-membered cycloalkyl or heterocycloalkyl; and 5-, to 10-membered monocyclic or bicyclic heterocyclyl; wherein R⁴ is optionally substituted with from 1 to 4 R¹⁴ groups;
R^{5a}, R^{5b}, R⁸ and R¹² are each independently selected at each occurrence from H, C₁-C₄-haloalkyl, cyclopropyl and C₁-C₄-alkyl;
R⁶ is independently selected from H, C₁-C₄-haloalkyl, C₁-C₄-alkyl and C₃-C₄-cycloalkyl;
R⁷ and R¹³ are each independently at each occurrence selected from H, C₁-C₄-alkyl, C₁-C₄-haloalkyl and C(O)—C₁-C₄-alkyl;
or R¹² and R¹³ together with the nitrogen to which they are attached form a ring system selected from: monocyclic 4- to 7-membered heterocycloalkyl group; and a fused, spirofused or bridged bicyclic 6- to 11-membered heterocycloalkyl group; said heterocycloalkyl group being optionally substituted with from 1 to 6 R^{10a} groups;
R⁹ is independently at each occurrence selected from oxo, halo, cyano, NR¹²R¹³, OR¹², COR¹², CO₂R¹², CONR¹²R¹², CONR¹²R¹³, C₁-C₄-alkyl, C₁-C₄-alkyl substituted with NR¹²R¹³, C₁-C₄-alkyl substituted with OR¹², C₁-C₄-alkyl substituted with cyano, C₂-C₄-alkenyl, C₂-C₄-alkynyl, C₁-C₄-haloalkyl and cyclopropyl;
R¹⁰ is independently at each occurrence selected from oxo, halo, cyano, NR¹²R¹³, OR¹², COR¹², CO₂R¹², CONR¹²R¹², C₁-C₄-alkyl, C₁-C₄-alkyl substituted with NR¹²R¹³, C₁-C₄-alkyl substituted with OR¹², C₁-C₄-alkyl substituted with cyano, C₁-C₄-alkyl substituted with phenyl, C₂-C₄-alkenyl, C₂-C₄-alkynyl, C₁-C₄-haloalkyl and cyclopropyl;
R^{10a} is independently at each occurrence selected from oxo, halo, cyano, NR¹²R^{13a}, OR¹², COR¹², CO₂R¹², CONR¹²R¹², CONR¹²R^{13a}, C₁-C₄-alkyl, C₁-C₄-alkyl substituted with NR¹²R^{13a} C₁-C₄-alkyl substituted with OR¹², C₁-C₄-alkyl substituted with cyano, C₁-C₄-alkyl substituted with phenyl, C₂-C₄-alkenyl, C₂-C₄-alkynyl, C₁-C₄-haloalkyl and cyclopropyl;
R^{13a} is independently at each occurrence selected from H, C₁-C₄-alkyl, C₁-C₄-haloalkyl and C(O)—C₁-C₄-alkyl;
R¹¹ is independently selected from halo, cyano, nitro, NR¹²R¹³, OR¹², CO₂R¹², CONR¹²R¹², C₁-C₄-alkyl, C₁-C₄-alkyl substituted with NR¹²R¹³, C₁-C₄-alkyl substituted with OR¹², monocyclic 4- to 7-membered cycloalkyl or heterocycloalkyl, C₂-C₄-alkenyl, C₂-C₄-alkynyl, C₁-C₄-haloalkyl and cyclopropyl;
R¹⁴ is independently at each occurrence selected from halo, cyano, nitro, NR¹²R¹³, OR¹², CO₂R¹², CONR¹²R¹², C₁-C₄-alkyl, C₁-C₄-alkyl substituted with NR¹²R¹³, C₁-C₄-alkyl substituted with OR¹², C₂-C₄-alkenyl, C₂-C₄-alkynyl, C₁-C₄-haloalkyl, phenyl and cyclopropyl;
wherein any of the aforementioned alkyl, alkylene, phenyl or cycloalkyl (e.g. cyclopropyl) groups is optionally substituted, where chemically possible, by 1 to 5 substituents which are each independently at each occurrence selected from the group consisting of: C₁-C₄-alkyl, C₁-C₄-alkyl substituted with OR^{a}, halo, nitro, cyano, NR^{a}R^{b}, OR^{a}, SR^{a}, CO₂R^{a}, C(O)R^{a}, CONR^{a}R^{a};
wherein R^{a} is independently at each occurrence selected from H, C₁-C₄-alkyl and C₁-C₄-haloalkyl; and R^{b} is independently at each occurrence selected from H, C₁-C₄-alkyl, C(O)—C₁-C₄-alkyl and S(O)₂—C₁-C₄-alkyl.

For the absence of doubt, the ring comprising X² and X³ is selected from pyrazole, isoxazole and isothiazole. Thus, either X² is N and X³ is NR⁶ or X³ is N and X² is selected from NR⁶, O and S.

In an embodiment, the compound of formula (I) is a compound of formula (II):

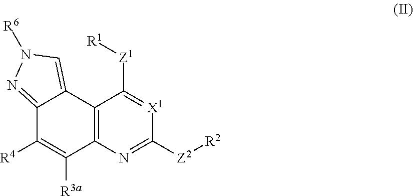

wherein R¹, R², R^{3a}, R⁴, R⁶, X¹, Z¹ and Z² are as described above for compounds of formula (I).

In an embodiment, the compound of formula (I) is a compound of formula (III):

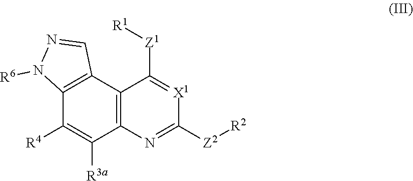

wherein R¹, R², R^{3a}, R⁴, R⁶, X¹, Z¹ and Z² are as described above for compounds of formula (I).

In an embodiment, the compound of formula (I) is a compound of formula (IV):

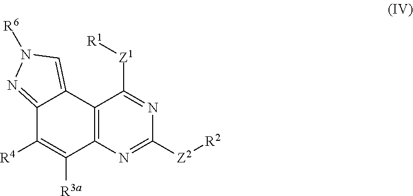

wherein R¹, R², R^{3a}, R⁴, R⁶, Z¹ and Z² are as described above for compounds of formula (I).

In an embodiment, the compound of formula (I) is a compound of formula (IVa):

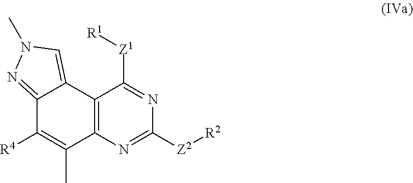

wherein R¹, R², R⁴, Z¹, and Z² are as described above for compounds of formula (I).

In an embodiment, the compound of formula (I) is a compound of formula (IVb):

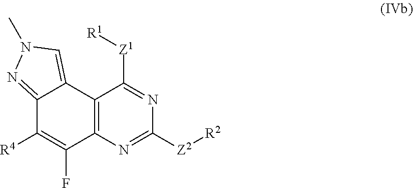

wherein R¹, R², R⁴, Z¹, and Z² are as described above for compounds of formula (I).

In an embodiment, the compound of formula (I) is a compound of formula (V):

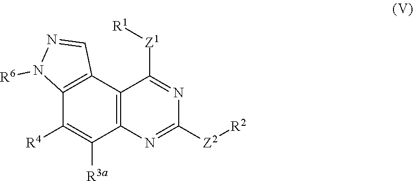

wherein R¹, R², R^{3a}, R⁴, R⁶, Z¹ and Z² are as described above for compounds of formula (I).

In an embodiment, the compound of formula (I) is a compound of formula (Va):

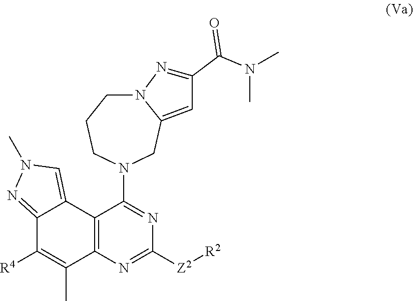

wherein R², R⁴, Z² are as described above for compounds of formula (I).

In an embodiment, the compound of formula (I) is a compound of formula (Vb):

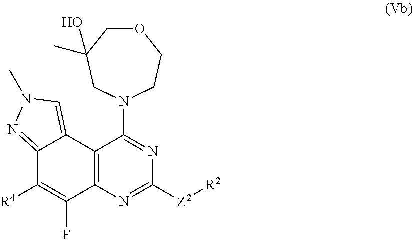

wherein R², R⁴, Z² are as described above for compounds of formula (I).

In an embodiment, the compound of formula (I) is a compound of formula (Vc):

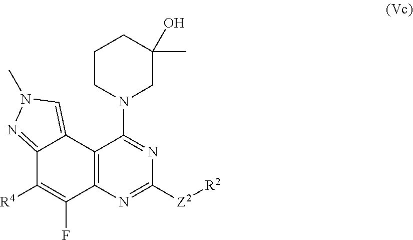

wherein R², R⁴, Z² are as described above for compounds of formula (I).

In an embodiment, the compound of formula (I) is a compound of formula (Vd):

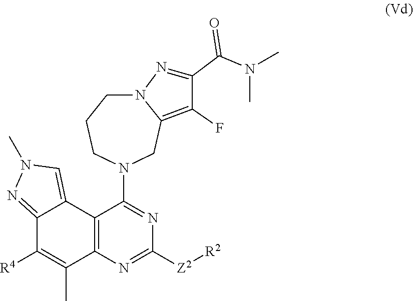

wherein R², R⁴, Z² are as described above for compounds of formula (I).

In an embodiment, the compound of formula (I) is a compound of formula (VI):

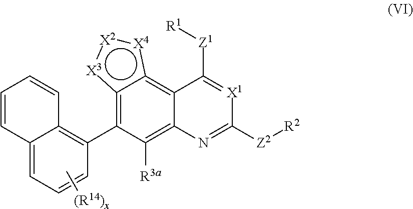

wherein R¹, R², R^{3a}, R¹⁴, X¹, X², X³, X⁴, Z¹ and Z² are as described above for compounds of formula (I); and x is independently selected from 0, 1, 2, 3, and 4. For the absence of doubt, throughout this specification, the x R¹⁴ groups may be attached to either ring of the naphthyl group.

In an embodiment, the compound of formula (I) is a compound of formula (VII):

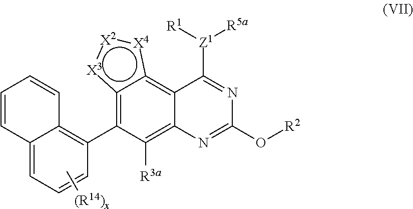

wherein R¹, R², R^{3a}, R^{5a}, R¹⁴, X², X³ and X⁴ are as described above for compounds of formula (I); and x is independently selected from 0, 1, 2, 3, and 4. For the absence of doubt, throughout this specification, the x R¹⁴ groups may be attached to either ring of the naphthyl group.

In an embodiment, the compound of formula (I) is a compound of formula (VIII):

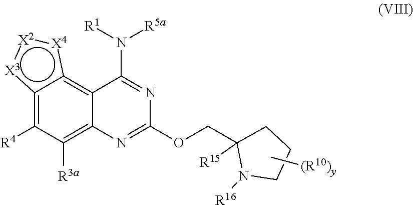

wherein R¹, R^{3a}, R⁴, R^{5a}, R¹⁰, X², X³ and X⁴ are as described above for compounds of formula (I); and wherein R¹⁵ is independently selected from H, C₁-C₄-alkyl; wherein R¹⁶ is independently selected from H, C₁-C₄-alkyl and cyclopropyl; or wherein R¹⁵ and R¹⁶ together with the atoms to which they are attached form a 5- or 6-membered heterocycloalkyl ring, optionally substituted with 1 or 2 R¹⁰ groups; and y is independently selected from 0, 1, 2, 3, and 4.

In an embodiment, the compound of formula (I) is a compound of formula (IX):

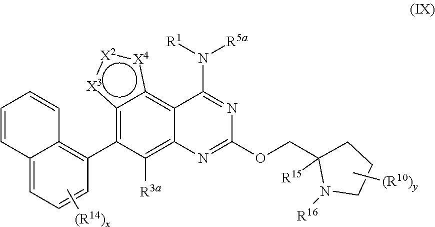

wherein R¹, R^{3a}, R^{5a}, R¹⁰, R¹⁴, X², X³ and X⁴ are as described above for compounds of formula (I); wherein R¹⁵ is independently selected from H, C₁-C₄-alkyl; wherein R¹⁶ is independently selected from H, C₁-C₄-alkyl and cyclopropyl; or wherein R¹⁵ and R¹⁶ together with the atoms to which they are attached form a 5- or 6-membered heterocycloalkyl ring, optionally substituted with 1 or 2 R¹⁰ groups; x is independently selected from 0, 1, 2, 3, and 4; and y is independently selected from 0, 1, 2, 3, and 4.

In an embodiment, the compound of formula (I) is a compound of formula (X):

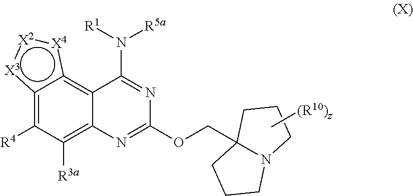

wherein R¹, R^{3a}, R⁴, R^{5a}, R¹⁰, X², X³ and X⁴ are as described above for compounds of formula (I); and wherein z is independently selected from 0, 1, 2, 3, and 4. For the absence of doubt, throughout this specification, the z R¹⁰ groups may be attached to either ring of the pyrrolizidinyl group.

In an embodiment, the compound of formula (I) is a compound of formula (XI):

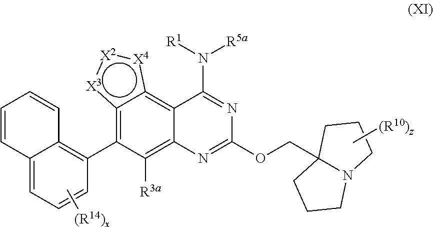

wherein R¹, R^{3a}, R^{5a}, R¹⁰, R¹⁴, X², X³ and X⁴ are as described above for compounds of formula (I); wherein x is independently selected from 0, 1, 2, 3, and 4; and wherein z is independently selected from 0, 1, 2, 3, and 4.

In an embodiment, the compound of formula (I) is a compound of formula (XII):

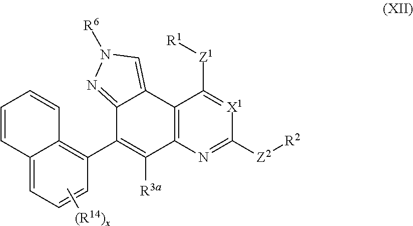

wherein R¹, R², R^{3a}, R⁶, R¹⁴, X¹, Z¹ and Z² are as described above for compounds of formula (I); and x is independently selected from 0, 1, 2, 3, and 4. For the absence of doubt, throughout this specification, the x R¹⁴ groups may be attached to either ring of the naphthyl group.

In an embodiment, the compound of formula (I) is a compound of formula (XIII):

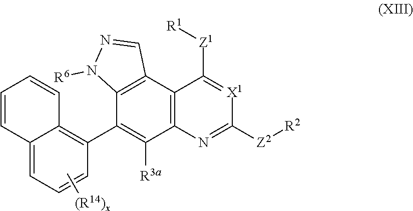

wherein R¹, R², R^{3a}, R⁶, R¹⁴, X¹, Z¹ and Z² are as described above for compounds of formula (I); and x is independently selected from 0, 1, 2, 3, and 4. For the absence of doubt, throughout this specification, the x R¹⁴ groups may be attached to either ring of the naphthyl group.

In an embodiment, the compound of formula (I) is a compound of formula (XIV):

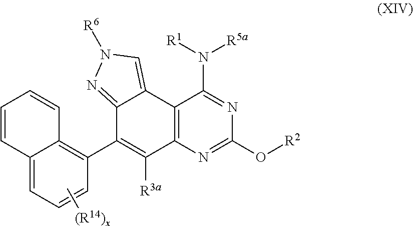

wherein R¹, R², R^{3a}, R⁶, R^{5a}, and R¹⁴ are as described above for compounds of formula (I);
and x is independently selected from 0, 1, 2, 3, and 4. For the absence of doubt, throughout this specification, the x R¹⁴ groups may be attached to either ring of the naphthyl group.

In an embodiment, the compound of formula (I) is a compound of formula (XV):

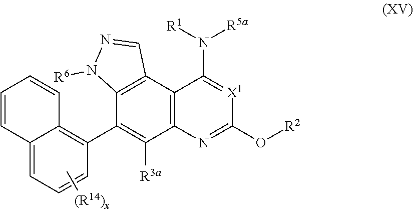

wherein R¹, R², R^{3a}, R⁶, R^{5a}, and R¹⁴ are as described above for compounds of formula (I);
and x is independently selected from 0, 1, 2, 3, and 4. For the absence of doubt, throughout this specification, the x R¹⁴ groups may be attached to either ring of the naphthyl group.

In an embodiment, the compound of formula (I) is a compound of formula (XVI):

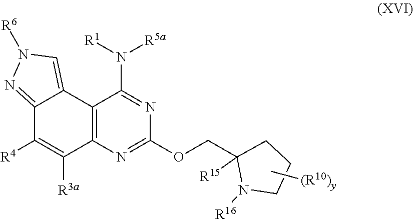

wherein R¹, R^{3a}, R⁴, R^{5a}, R⁶ andR10 are as described above for compounds of formula (I); and
wherein R¹⁵ is independently selected from H, C₁-C₄-alkyl; wherein R¹⁶ is independently selected from H, C₁-C₄-alkyl and cyclopropyl; or wherein R¹⁵ and R¹⁶ together with the atoms to which they are attached form a 5- or 6-membered heterocycloalkyl ring, optionally substituted with 1 or 2 R¹⁰ groups; and y is independently selected from 0, 1, 2, 3, and 4.

In an embodiment, the compound of formula (I) is a compound of formula (XVII):

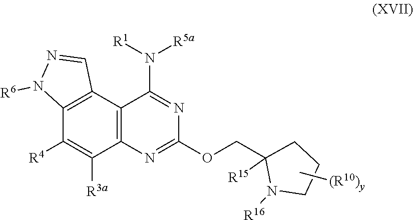

wherein R¹, R^{3a}, R⁴, R^{5a}, R⁶ and R¹⁰ are as described above for compounds of formula (I); and
wherein R¹⁵ is independently selected from H, C₁-C₄-alkyl; wherein R¹⁶ is independently selected from H, C₁-C₄-alkyl and cyclopropyl; or wherein R¹⁵ and R¹⁶ together with the atoms to which they are attached form a 5- or 6-membered heterocycloalkyl ring, optionally substituted with 1 or 2 R¹⁰ groups; and y is independently selected from 0, 1, 2, 3, and 4.

In an embodiment, the compound of formula (I) is a compound of formula (XVIII):

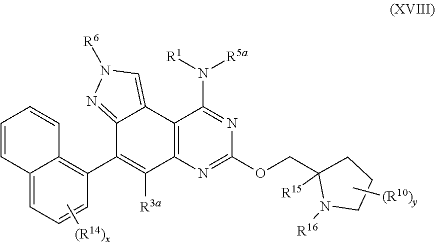

wherein R¹, R^{3a}, R^{5a}, R⁶, R¹⁰, and R¹⁴ are as described above for compounds of formula (I);
wherein R¹⁵ is independently selected from H, C₁-C₄-alkyl; wherein R¹⁶ is independently selected from H, C₁-C₄-alkyl and cyclopropyl; or wherein R¹⁵ and R¹⁶ together with the atoms to which they are attached form a 5- or 6-membered heterocycloalkyl ring, optionally substituted with 1 or 2 R¹⁰ groups; x is independently selected from 0, 1, 2, 3, and 4; and y is independently selected from 0, 1, 2, 3, and 4.

In an embodiment, the compound of formula (I) is a compound of formula (XIX):

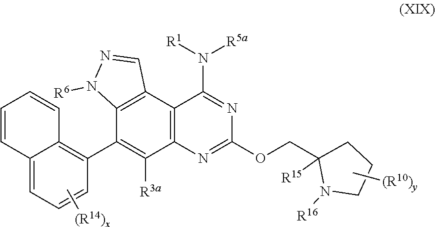

wherein R¹, R^{3a}, R^{5a}, R⁶, R¹⁰, and R¹⁴ are as described above for compounds of formula (I);
wherein R¹⁵ is independently selected from H, C₁-C₄-alkyl; wherein R¹⁶ is independently selected from H, C₁-C₄-alkyl and cyclopropyl; or wherein R¹⁵ and R¹⁶ together with the atoms to which they are attached form a 5- or 6-membered heterocycloalkyl ring, optionally substituted with 1 or 2 R¹⁰ groups; x is independently selected from 0, 1, 2, 3, and 4; and y is independently selected from 0, 1, 2, 3, and 4.

In an embodiment, the compound of formula (I) is a compound of formula (XX):

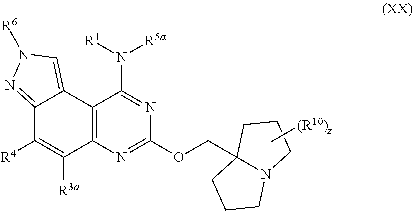

wherein R¹, R^{3a}, R⁴, R^{5a}, R⁶ and R¹⁰ are as described above for compounds of formula (I); and
wherein z is independently selected from 0, 1, 2, 3, and 4. For the absence of doubt, throughout this specification, the z R¹⁰ groups may be attached to either ring of the pyrrolizidinyl group.

In an embodiment, the compound of formula (I) is a compound of formula (XXI):

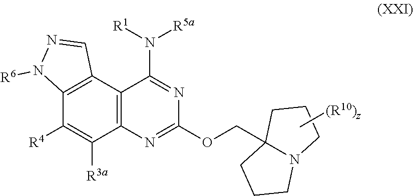

wherein R¹, R^{3a}, R⁴, R^{5a}, R⁶ and R¹⁰ are as described above for compounds of formula (I); and
wherein z is independently selected from 0, 1, 2, 3, and 4. For the absence of doubt, throughout this specification, the z R¹⁰ groups may be attached to either ring of the pyrrolizidinyl group.

In an embodiment, the compound of formula (I) is a compound of formula (XXII):

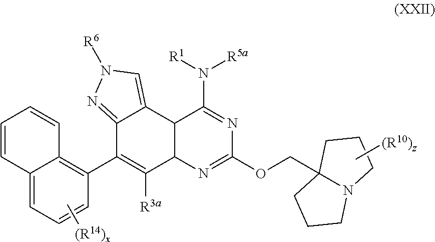

wherein R¹, R^{3a}, R^{5a}, R⁶, R¹⁰, and R¹⁴ are as described above for compounds of formula (I);
wherein x is independently selected from 0, 1, 2, 3, and 4; and wherein z is independently selected from 0, 1, 2, 3, and 4.

In an embodiment, the compound of formula (I) is a compound of formula (XXIII):

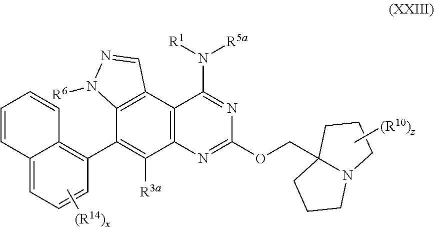

wherein R¹, R^{3a}, R^{5a}, R⁶, R¹⁰, and R¹⁴ are as described above for compounds of formula (I);
wherein x is independently selected from 0, 1, 2, 3, and 4; and wherein z is independently selected from 0, 1, 2, 3, and 4.

In an embodiment, the compound of formula (I) is a compound of formula (XXIV):

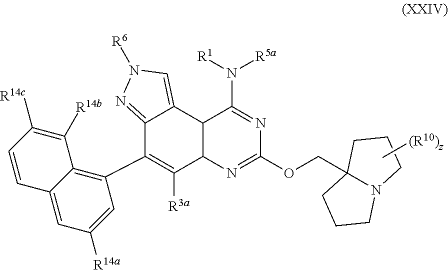

wherein R¹, R^{3a}, R^{5a}, R⁶, and R¹⁰ are as described above for compounds of formula (I); wherein z is independently selected from 0, 1, 2, 3, and 4; and wherein R^{14a} is OR¹² e.g., OH; R^{14b} is C₁-C₄-alkyl e.g., methyl or ethyl; and R^{14c} is halo e.g, F.

In an embodiment, the compound of formula (I) is a compound of formula (XXV):

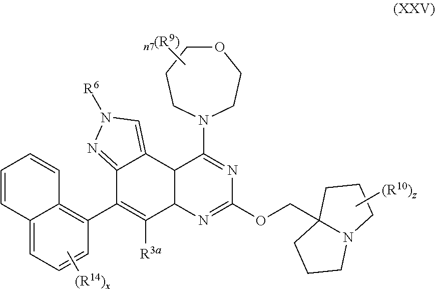

wherein R^{3a}, R⁶, R¹⁰, R⁹ and R¹⁴ are as described above for compounds of formula (I); wherein x is independently selected from 0, 1, 2, 3, and 4; wherein n10 is independently selected from 0, 1 and 2; and wherein z is independently selected from 0, 1, 2, 3, and 4. n6 may be 0. n6 may be 1. n6 may be 2.

In an embodiment, the compound of formula (I) is a compound of formula (XXVI):

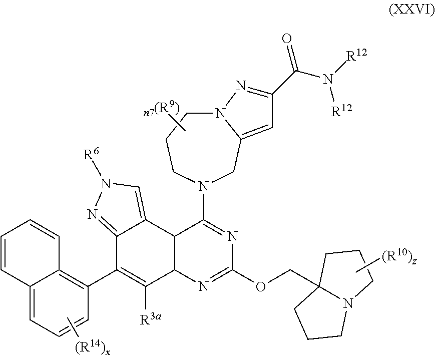

wherein R^{3a}, R⁶, R⁹, R¹⁰ and R¹⁴ are as described above for compounds of formula (I); wherein R¹² is independently at each occurrence selected from H, and C₁-C₄-alkyl, e.g., methyl;
wherein x is independently selected from 0, 1, 2, 3, and 4; wherein n7 is independently selected from 0 and 1; and wherein z is independently selected from 0, 1, 2, 3, and 4. n7 may be 0.

The following embodiments apply to compounds of any of formulae (I)-(XXIII). These embodiments are independent and interchangeable. Any one embodiment may be combined with any other embodiment, where chemically allowed. In other words, any of the features described in the following embodiments may (where chemically allowable) be combined with the features described in one or more other embodiments. In particular, where a compound is exemplified or illustrated in this specification, any two or more of the embodiments listed below, expressed at any level of generality, which encompass that compound may be combined to provide a further embodiment which forms part of the present disclosure.

X¹ may be N. X¹ may be CR^{3b}.

The ring comprising X² and X³ may be pyrazole, The ring comprising X² and X³ may be selected from isoxazole and isothiazole. It may be that X² is N and X³ is NR⁶. It may be that X³ is N and X² is selected from NR⁶, O and S. It may be that X³ is N and X² is NR⁶. It may be that X³ is N and X² is selected from O and S.

Z¹ may be —O—. Z¹ may be —NR^{5a}—.

Z² may be —O—. Z² may be —NR^{5b}—.

R¹ is independently C₀-C₃-alkylene-R^{1a} wherein R^{1a} is independently selected from a 4- to 7-membered heterocycloalkyl ring; a phenyl ring; and a C₃-C₇-cycloalkyl ring optionally substituted with an NR⁷R⁸ group; wherein said heterocycloalkyl ring, said phenyl ring or said cycloalkyl ring is optionally substituted with from 1 to 4 R⁹ groups.

R¹ is independently C₀-C₃-alkylene-R^{1a} wherein R^{1a} is independently selected from a 4- to 7-membered heterocycloalkyl ring; and a C₃-C₇-cycloalkyl ring optionally substituted with an NR⁷R⁸ group; wherein said heterocycloalkyl ring or said cycloalkyl ring is optionally substituted with from 1 to 4 R⁹ groups.

R¹ may be C₀-C₃-alkylene-R^{1a}. R¹ may be C₀-C₃-alkylene-R^{1a} wherein R^{1a} is independently selected from an oxygen containing 4- to 7-membered heterocycloalkyl ring, a 4- to 7-membered heterocycloalkyl ring; and a C₃-C₇-cycloalkyl ring optionally substituted with an NR⁷R⁸ group; wherein said heterocycloalkyl ring or said cycloalkyl ring is optionally substituted with from 1 to 4 R⁹ groups. R¹ may be CH₂-R^{1a} wherein R^{1a} is independently selected from a 4- to 7-membered heterocycloalkyl ring; and a C₃-C₇-cycloalkyl ring optionally substituted with an NR⁷R⁸ group; wherein said heterocycloalkyl ring or said cycloalkyl ring is optionally substituted with from 1 to 4 R⁹ groups.

R¹ may be C₀-C₃-alkylene-R^{1a} wherein R^{1a} is independently selected from an oxygen containing 4- to 7-membered heterocycloalkyl ring, a nitrogen containing 4- to 7-membered heterocycloalkyl ring; and a C₃-C₇-cycloalkyl ring optionally substituted with an NR⁷R⁸ group; wherein said heterocycloalkyl ring or said cycloalkyl ring is optionally substituted with from 1 to 4 R⁹ groups. R¹ may be CH₂—R^{1a} wherein R^{1a} is independently selected from a nitrogen containing 4- to 7-membered heterocycloalkyl ring; and a C₃-C₇-cycloalkyl ring optionally substituted with an NR⁷R⁸ group; wherein said heterocycloalkyl ring or said cycloalkyl ring is optionally substituted with from 1 to 4 R⁹ groups.

R¹ may be R^{1a} wherein R^{1a} is independently selected from a 4- to 7-membered heterocycloalkyl ring; and a C₃-C₇-cycloalkyl ring optionally substituted with an NR⁷R⁸ group; wherein said heterocycloalkyl ring or said cycloalkyl ring is optionally substituted with from 1 to 4 R⁹ groups.

R¹ may be R^{1a} wherein R^{1a} is independently selected from a nitrogen containing 4- to 7-membered heterocycloalkyl ring; and a C₃-C₇-cycloalkyl ring optionally substituted with an NR⁷R⁸ group; wherein said heterocycloalkyl ring or said cycloalkyl ring is optionally substituted with from 1 to 4 R⁹ groups. R¹ may be R^{1a} wherein R^{1a} is an oxygen containing 4- to 7-membered heterocycloalkyl ring; wherein said heterocycloalkyl ring is optionally substituted with from 1 to 4 R⁹ groups. R¹ may be R^{1a} wherein R^{1a} is an oxygen containing 4- to 7-membered heterocycloalkyl ring e.g. a tetrahydropyranyl ring.

R¹ is independently C₀-C₃-alkylene-R^{1a} wherein R^{1a} is independently selected from a 4- to 7-membered heterocycloalkyl ring; and a C₃-C₇-cycloalkyl ring substituted with an NR⁷R⁸ group; wherein said heterocycloalkyl ring or said cycloalkyl ring is optionally substituted with from 1 to 4 R⁹ groups.

R¹ may be C₀-C₃-alkylene-R^{1a}. R¹ may be C₀-C₃-alkylene-R^{1a} wherein R^{1a} is independently selected from an oxygen containing 4- to 7-membered heterocycloalkyl ring, a 4- to 7-membered heterocycloalkyl ring; and a C₃-C₇-cycloalkyl ring substituted with an NR⁷R⁸ group; wherein said heterocycloalkyl ring or said cycloalkyl ring is optionally substituted with from 1 to 4 R⁹ groups. R¹ may be CH₂—R^{1a} wherein R^{1a} is independently selected from a 4- to 7-membered heterocycloalkyl ring; and a C₃-C₇-cycloalkyl ring substituted with an NR⁷R⁸ group; wherein said heterocycloalkyl ring or said cycloalkyl ring is optionally substituted with from 1 to 4 R⁹ groups.

R¹ may be C₀-C₃-alkylene-R^{1a} wherein R^{1a} is independently selected from an oxygen containing 4- to 7-membered heterocycloalkyl ring, a nitrogen containing 4- to 7-membered heterocycloalkyl ring; and a C₃-C₇-cycloalkyl ring substituted with an NR⁷R⁸ group; wherein said heterocycloalkyl ring or said cycloalkyl ring is optionally substituted with from 1 to 4 R⁹ groups. R¹ may be CH₂—R^{1a} wherein R^{1a} is independently selected from a nitrogen containing 4- to 7-membered heterocycloalkyl ring; and a C₃-C₇-cycloalkyl ring substituted with an NR⁷R⁸ group; wherein said heterocycloalkyl ring or said cycloalkyl ring is optionally substituted with from 1 to 4 R⁹ groups.

R¹ may be R^{1a} wherein R^{1a} is independently selected from a 4- to 7-membered heterocycloalkyl ring; and a C₃-C₇-cycloalkyl ring substituted with an NR⁷R⁸ group; wherein said heterocycloalkyl ring or said cycloalkyl ring is optionally substituted with from 1 to 4 R⁹ groups.

R¹ may be R^{1a} wherein R^{1a} is independently selected from a nitrogen containing 4- to 7-membered heterocycloalkyl ring; and a C₃-C₇-cycloalkyl ring substituted with an NR⁷R⁸ group; wherein said heterocycloalkyl ring or said cycloalkyl ring is optionally substituted with from 1 to 4 R⁹ groups. R¹ may be R^{1a} wherein R^{1a} is an oxygen containing 4- to 7-membered heterocycloalkyl ring; wherein said heterocycloalkyl ring is optionally substituted with from 1 to 4 R⁹ groups. R¹ may be R^{1a} wherein R^{1a} is an oxygen containing 4- to 7-membered heterocycloalkyl ring e.g. a tetrahydropyranyl ring.

R¹ may be C₀-C₃-alkylene-R^{1a} wherein R^{1a} is a 4- to 7-membered heterocycloalkyl ring; wherein said heterocycloalkyl ring is optionally substituted with from 1 to 4 R⁹ groups. R¹ may be CH₂-alkylene-R^{1a} wherein R^{1a} is a 4- to 7-membered heterocycloalkyl ring; wherein said heterocycloalkyl ring is optionally substituted with from 1 to 4 R⁹ groups.

R¹ may be C₀-C₃-alkylene-R^{1a} wherein R^{1a} is a nitrogen containing 4- to 7-membered heterocycloalkyl ring; wherein said heterocycloalkyl ring is optionally substituted with from 1 to 4 R⁹ groups. R¹ may be CH₂-alkylene-R^{1a} wherein R^{1a} is a nitrogen containing 4- to 7-membered heterocycloalkyl ring; wherein said heterocycloalkyl ring is optionally substituted with from 1 to 4 R⁹ groups.

R¹ may be C₀-C₃-alkylene-R^{1a} wherein R^{1a} is a 4- to 7-membered heterocycloalkyl ring wherein the ring does not comprise any nitrogen atoms; wherein said heterocycloalkyl ring is optionally substituted with from 1 to 4 R⁹ groups. R¹ may be CH₂-alkylene-R^{1a} wherein R^{1a} is a 4- to 7-membered heterocycloalkyl ring wherein the ring does not comprise any nitrogen atoms; wherein said heterocycloalkyl ring is optionally substituted with from 1 to 4 R⁹ groups.

R¹ may be R^{1a} wherein R^{1a} is a 4- to 7-membered heterocycloalkyl ring; wherein said heterocycloalkyl ring is optionally substituted with from 1 to 4 R⁹ groups. R¹ may be R^{1a} wherein R^{1a} is a nitrogen containing 4- to 7-membered heterocycloalkyl ring; wherein said heterocycloalkyl ring is optionally substituted with from 1 to 4 R⁹ groups. R¹ may be R^{1a} wherein R^{1a} is a 4- to 7-membered heterocycloalkyl ring; wherein said heterocycloalkyl ring is optionally substituted with from 1 to 4 R⁹ groups wherein the ring does not comprise any nitrogen atoms.

R¹ may be C₀-C₃-alkylene-R^{1a} wherein R^{1a} is a C₃-C₇-cycloalkyl ring substituted with an NR⁷R⁸ group; wherein said cycloalkyl ring is optionally substituted with from 1 to 4 R⁹ groups. R¹ may be CH₂-alkylene-R^{1a} wherein R^{1a} is a C₃-C₇-cycloalkyl ring substituted with an NR⁷R⁸ group; wherein said cycloalkyl ring is optionally substituted with from 1 to 4 R⁹ groups.

R¹ may be R^{1a} wherein R^{1a} is a C₃-C₇-cycloalkyl ring optionally substituted with an NR⁷R⁸ group; wherein said cycloalkyl ring is optionally substituted with from 1 to 4 R⁹ groups.

R¹ may be R^{1a} wherein R^{1a} is a C₃-C₇-cycloalkyl ring substituted with an NR⁷R⁸ group; wherein said cycloalkyl ring is optionally substituted with from 1 to 4 R⁹ groups.

R¹ may be R^{1a} wherein R^{1a} is phenyl optionally substituted with from 1 to 4 R⁹ groups. R¹ may be R^{1a} wherein R^{1a} is unsubstituted phenyl.

R¹ may be C₂-C₆-alkylene-R^{1b}. R¹ may be C₂-C₃-alkylene-R^{1b}. R¹ may be C₃-alkylene-R^{1b}. R^{1b} may be independently selected from: NR⁷R⁸, OR⁸ and SR⁸. R^{1b} may be OR⁸. R^{1b} may be SR⁸. R^{1b} may be NR⁷R⁸. R⁸ may be C₁-C₄-alkyl, e.g. Me.

It may be that R¹ and R^{5a} are selected such that NR¹R^{5a} comprises no more than a single amine, wherein said single amine may be a primary, secondary or tertiary amine. It may be that R¹ and R^{5a} are selected such that NR¹R^{5a} comprises no more than a single secondary or tertiary amine, wherein said single amine may be a secondary or tertiary amine. Compounds having no more than a single amine at this position surprisingly exhibit broad spectrum inhibition across a range of mutant KRAS forms as well as wild type KRAS rather than inhibition of the specific KRAS G12C and G12D proteins. Compounds having a single amine in this portion of the molecule typically exhibit broad spectrum inhibition of KRAS mutants including KRAS G12D, KRAS G12C, KRAS G12V, KRAS G12A, KRAS G13D and KRAS Q61H as well as wild-type KRAS. As such these compounds may be of therapeutic benefit in treating cancers bearing KRAS mutations beyond G12D and G12C, as well as cancers dependent on wild type KRAS.

It may be that R¹ and R^{5a} are selected such that NR¹R^{5a} comprises more than one amine, wherein said amines may be a primary, secondary or tertiary amine. It may be that R¹ and R^{5a} are selected such that NR¹R^{5a} comprises more than one amine, wherein said amines may be a secondary or tertiary amine. It may be that R¹ and R^{5a} are selected such that NR¹R^{5a} comprises two amines, wherein said amines may be a primary, secondary or tertiary amine. It may be that R¹ and R^{5a} are selected such that NR¹R^{5a} comprises two amines, wherein said amines may be a secondary or tertiary amine. Compounds in which R¹ and R^{5a} are selected such that NR¹R^{5a} comprises more than a single amine, e.g. two amines, typically inhibit KRAS G12D selectively.

It may be that R¹ and R^{5a} together with the nitrogen to which they are attached form a ring system selected from: a monocyclic 4- to 7-membered heterocycloalkyl group, optionally substituted with from 1 to 4 R⁹ groups; a fused or spirofused bicyclic 6- to 11-membered heterocyclyl group, optionally substituted with from 1 to 4 R⁹ groups; and a bridged bicyclic 6- to 11-membered heterocycloalkyl group, optionally substituted with from 1 to 4 R⁹ groups; wherein the nitrogen to which R¹ and R^{5a} are attached is the only heteroatom in the ring system.

It may be that R¹ and R^{5a} together with the nitrogen to which they are attached form a ring system selected from: a monocyclic 4- to 7-membered heterocycloalkyl group, optionally substituted with from 1 to 4 R⁹ groups; a fused or spirofused bicyclic 6- to 11-membered heterocyclyl group, optionally substituted with from 1 to 4 R⁹ groups; and a bridged bicyclic 6- to 11-membered heterocycloalkyl group, optionally substituted with from 1 to 4 R⁹ groups; wherein the nitrogen to which R¹ and R^{5a} are attached is the only nitrogen in the ring system.

It may be that R¹ and R^{5a} are selected such that the nitrogen of NR¹R^{5a} is the nitrogen of the single amine. It may be that R¹ and R^{5a} are selected such that NR¹R^{5a} is the single amine. For the avoidance of doubt, the term “amine” as used herein encompasses primary amines, e.g., methylamine; secondary amines, e.g., dimethylamine; tertiary amines, e.g., trimethylamine; cyclic amines, e.g., piperidine. For the avoidance of doubt, the term “amine” as used herein excludes amides and lactams, e.g., piperazinonyl.

It may be that R¹ and R^{5a} together with the nitrogen to which they are attached form a ring system having the structure:

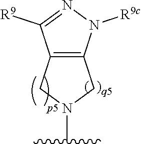

wherein R^{9c} is selected from H and C₁-C₄-alkyl, p5 and q5 and are each selected from 0, 1, 2 and 3; providing that the sum of p5 and q5 is 1 or greater.

It may be that R¹ and R^{5a} together with the nitrogen to which they are attached form a ring system having a structure selected from:

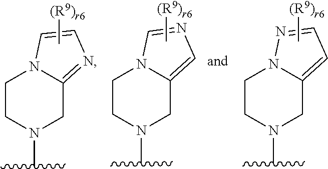

wherein r6 is selected from 0, 1 and 2.

It may be that R¹ and R^{5a} together with the nitrogen to which they are attached form a ring system having a structure selected from:

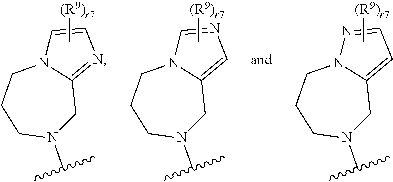

wherein r7 is selected from 0, 1 and 2.

It may be that R¹ and R^{5a} together with the nitrogen to which they are attached form a ring system selected from: a monocyclic 4- to 7-membered group heterocycloalkyl group, optionally substituted with from 1 to 4 R⁹ groups; a fused, spirofused or bridged bicyclic 6- to 11-membered heterocyclyl group, optionally substituted with from 1 to 4 R⁹ groups.

It may be that R¹ and R^{5a} together with the nitrogen to which they are attached form a ring system selected from: a monocyclic 4- to 7-membered group heterocycloalkyl group, optionally substituted with from 1 to 4 R⁹ groups; a fused, spirofused or bridged bicyclic 6- to 11-membered heterocyclyl group, optionally substituted with from 1 to 4 R⁹ groups; wherein the ring system does not comprise a nitrogen other than the nitrogen to which R¹ and R^{5a} are attached.

It may be that R¹ and R^{5a} together with the nitrogen to which they are attached form a ring system selected from: a monocyclic 4- to 7-membered group heterocycloalkyl group, optionally substituted with from 1 to 4 R⁹ groups; a fused or spirofused bicyclic 6- to 11-membered heterocyclyl group, optionally substituted with from 1 to 4 R⁹ groups; and a bridged bicyclic 6- to 11-membered heterocycloalkyl group, optionally substituted with from 1 to 4 R⁹ groups.

It may be that R¹ and R^{5a} together with the nitrogen to which they are attached form a ring system selected from: a monocyclic 4- to 7-membered group heterocycloalkyl group, optionally substituted with from 1 to 4 R⁹ groups; a fused or spirofused bicyclic 6- to 11-membered heterocyclyl group, optionally substituted with from 1 to 4 R⁹ groups.

It may be that R¹ and R^{5a} together with the nitrogen to which they are attached form a 6 or 7-membered group heterocycloalkyl group, optionally substituted with from 1 to 4 R⁹ groups. It may be that R¹ and R^{5a} together with the nitrogen to which they are attached form a 6 or 7-membered group heterocycloalkyl group, optionally substituted with from 1 to 4 R⁹ groups, wherein the total number of heteroatoms in the 6 or 7-membered group heterocycloalkyl group is 1 or 2. The total number of heteroatoms may be 2. It may be that R¹ and R^{5a} together with the nitrogen to which they are attached form a 6 or 7-membered group heterocycloalkyl group, optionally substituted with 1 R⁹ group.

It may be that R¹ and R^{5a} together with the nitrogen to which they are attached form a monocyclic 4- to 7-membered group heterocycloalkyl group, optionally substituted with from 1 to 4 R⁹ groups. It may be that R¹ and R^{5a} together with the nitrogen to which they are attached form an unsubstituted monocyclic 4- to 7-membered group heterocycloalkyl group. It may be that there is at least one R⁹ group and that at least one of said R⁹ groups is selected from NR¹²R¹³ and C₁-C₄-alkyl substituted with NR¹²R¹³. It may be that R¹ and R^{5a} together with the nitrogen to which they are attached form a ring system having the structure:

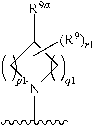

wherein R^{9a} is selected from NR¹²R¹³ and C₁-C₄-alkyl substituted with NR¹²R¹³; p1 is selected from 0, 1, 2 and 3, q1 is selected from 0, 1 and 2; and r1 is selected from 0, 1, 2 and 3. R¹ may be 0. R⁹ may independently at each occurrence be methyl. R^{9a} may be selected from NHR¹² and C₁-C₄-alkyl substituted with NHR¹².

It may be that R¹ and R^{5a} together with the nitrogen to which they are attached form a monocyclic 4- to 7-membered group heterocycloalkyl group comprising two nitrogen atoms in the ring, optionally substituted with from 1 to 4 R⁹ groups.

It may be that R¹ and R^{5a} together with the nitrogen to which they are attached form a ring system having the structure:

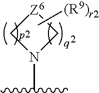

wherein Z⁶ is independently selected from C(O)NR^{9b}, NR^{9b}, O, S, S(O)₂, S(O), S(O)(NR^{9b}) and S(O)(NH); R^{9b} is selected from H and C₁-C₄-alkyl; p2 is selected from 2 and 3, q2 is 2; and r2 is selected from 0, 1, 2 and 3. Z⁶ may be selected from NR^{9b}, O, S, S(O)₂, S(O) and S(O)(NH). Z⁶ may be selected from C(O)NR^{9b}, O, S, S(O)₂, S(O), S(O)(NR^{9b}) and S(O)(NH). Z⁶ may be selected from O, S, S(O)₂, S(O) and S(O)(NH).
Z⁶ may be selected from NR^{9b}, O and S. Z⁶ may be selected from O and S. Z⁶ may be O.

It may be that R¹ and R^{5a} together with the nitrogen to which they are attached form a ring system having the structure:

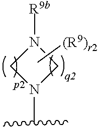

wherein R^{9b} is selected from H and C₁-C₄-alkyl; p2 is selected from 2 and 3, q2 is 2; and r2 is selected from 0, 1, 2 and 3. R² may be 0. R⁹ may independently at each occurrence be methyl. R^{9b} may be H. R^{9b} may be C₁-C₄-alkyl.

It may be that R¹ and R^{5a} together with the nitrogen to which they are attached form a fused or spirofused bicyclic 6- to 11-membered heterocyclyl group, optionally substituted with from 1 to 4 R⁹ groups. It may be that R¹ and R^{5a} together with the nitrogen to which they are attached form a fused or spirofused bicyclic 6- to 11-membered heterocyclyl group comprising two nitrogen atoms in the ring system, optionally substituted with from 1 to 4 R⁹ groups.

It may be that R¹ and R^{5a} together with the nitrogen to which they are attached form a spirofused bicyclic 6- to 11-membered heterocycloalkyl group, optionally substituted with from 1 to 4 R⁹ groups. It may be that R¹ and R^{5a} together with the nitrogen to which they are attached form a spirofused bicyclic 6- to 11-membered heterocycloalkyl group comprising two nitrogen atoms in the ring system, optionally substituted with from 1 to 4 R⁹ groups. It may be that R¹ and R^{5a} together with the nitrogen to which they are attached form a ring system having the structure:

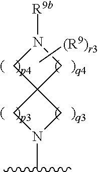

wherein R^{9b} is selected from H and C₁-C₄-alkyl; p3, p4, q3 and q4 are each independently selected from 0, 1, 2 and 3; providing that the sum of p3, p4, q3 and q4 is from 3 to 8, the sum of p3 and q3 is 2 or greater, and the sum of p4 and q4 is 2 or greater; and r3 is selected from 0, 1, 2 and 3. For the absence of doubt, throughout this specification, the r3 R⁹ groups may be attached to either ring of the spirofused bicyclic ring system. R³ may be 0. R⁹ may independently at each occurrence be methyl. R^{9b} may be H.

It may be that R¹ and R^{5a} together with the nitrogen to which they are attached form a fused bicyclic 6- to 11-membered heterocyclyl group, optionally substituted with from 1 to 4 R⁹ groups. It may be that R¹ and R^{5a} together with the nitrogen to which they are attached form a fused bicyclic 6- to 11-membered heterocyclyl group comprising two nitrogen atoms in the ring system, optionally substituted with from 1 to 4 R⁹ groups. It may be that R¹ and R^{5a} together with the nitrogen to which they are attached form a ring system having the structure:

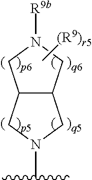

wherein R^{9b} is selected from H and C₁-C₄-alkyl; p5, p6, q5 and are each selected from 0, 1, 2 and 3; providing that the sum of p3, p4, q3 and q4 is from 2 to 7, the sum of p5 and q5 is 1 or greater, and the sum of p6 and q6 is 1 or greater; and r5 is selected from 0, 1, 2 and 3. For the absence of doubt, throughout this specification, the r5 R⁹ groups may be attached to either ring of the fused bicyclic ring system. R⁵ may be 0. R⁹ may independently at each occurrence be methyl. R^{9b} may be H.

It may be that R¹ and R^{5a} together with the nitrogen to which they are attached form a bridged bicyclic 6- to 11-membered heterocycloalkyl group, optionally substituted with from 1 to 4 R⁹ groups.

It may be that R¹ and R^{5a} together with the nitrogen to which they are attached form a fused bicyclic 6- to 11-membered heterocycloalkyl group, optionally substituted with from 1 to 4 R⁹ groups.

It may be that R¹ and R^{5a} together with the nitrogen to which they are attached form a ring system selected from: a monocyclic 4- to 7-membered group heterocycloalkyl group, optionally substituted with from 1 to 4 R⁹ groups; a fused or spirofused bicyclic 6- to 11-membered heterocycloalkyl group, optionally substituted with from 1 to 4 R⁹ groups; and a bridged bicyclic 6- to 11-membered heterocycloalkyl group, optionally substituted with from 1 to 4 R⁹ groups, wherein the bridged bicyclic 6- to 11-membered heterocycloalkyl group is not:

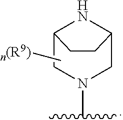

It may be that R¹ and R^{5a} together with the nitrogen to which they are attached form a bridged bicyclic 6- to 11-membered heterocycloalkyl group, optionally substituted with from 1 to 4 R⁹ groups, wherein the bridged bicyclic 6- to 11-membered heterocycloalkyl group is not:

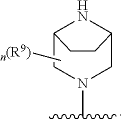

It may be that R¹ and R^{5a} together with the nitrogen to which they are attached form a ring system having the structure:

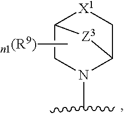

wherein Y¹ is independently selected from C(O)NR^{9d}, O and NR¹⁷; Z³ is independently selected from: CH₂, CH₂CH₂, CH₂—O—CH₂CH₂, CH₂—O—CH₂, CH₂—NR¹⁷—CH₂CH₂ and CH₂—NR¹⁷—CH₂; R¹⁷ is independently at each occurrence selected from H, C₁-C₄-haloalkyl, and C₁-C₄-alkyl; R^{9d} is independently selected from H and C₁-C₄-alkyl; and n1 is an integer selected from 0, 1, 2, 3 and 4. For the absence of doubt, throughout this specification, the n1 R⁹ groups may be attached to either ring of the bridged bicyclic ring system. Z³ may be independently selected from: CH₂, CH₂CH₂, CH₂—O—CH₂CH₂, CH₂—O—CH₂, Y¹ may be independently selected from O and NR¹⁷. Y¹ may be NR¹⁷. Y¹ may be NH. n1 may be 0. R⁹ may independently at each occurrence be methyl.

It may be that R¹ and R^{5a} together with the nitrogen to which they are attached form a ring system having the structure:

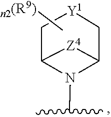

wherein Y¹ is independently selected from C(O)NR⁹d, O and NR¹⁷; Z⁴ is independently selected from: CH₂, CH₂CH₂, CH₂—O—CH₂CH₂, CH₂—O—CH₂, CH₂—NR¹⁷—CH₂CH₂ and CH₂—NR¹⁷—CH₂; R¹⁷ is independently at each occurrence selected from H, C₁-C₄-haloalkyl, and C₁-C₄-alkyl; Rod is independently selected from H and C₁-C₄-alkyl; and n2 is an integer selected from 0, 1, 2, 3 and 4. For the absence of doubt, throughout this specification, the n2 R⁹ groups may be attached to either ring of the bridged bicyclic ring system. Z⁴ may be independently selected from: CH₂, CH₂CH₂, CH₂—O—CH₂CH₂, CH₂—O—CH₂, Y¹ may be independently selected from O and NR¹⁷. Y¹ may be NR¹⁷. Y¹ may be NH. n2 may be 0. R⁹ may independently at each occurrence be methyl.

It may be that R¹ and R^{5a} together with the nitrogen to which they are attached form a ring system having the structure:

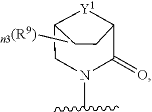

wherein Y¹ is independently selected from C(O)NR^{9d}, O and NR¹⁷; R¹⁷ is independently at each occurrence selected from H, C₁-C₄-haloalkyl, and C₁-C₄-alkyl; R^{9d} is independently selected from H and C₁-C₄-alkyl; and n3 is an integer selected from 0, 1, 2, 3 and 4. For the absence of doubt, throughout this specification, the n3 R⁹ groups may be attached to either ring of the bridged bicyclic ring system. Y¹ may be independently selected from O and NR¹⁷. Y¹ may be NR¹⁷. Y¹ may be NH. n3 may be 0. R⁹ may independently at each occurrence be methyl.

It may be that R¹ and R^{5a} together with the nitrogen to which they are attached form a ring system having the structure:

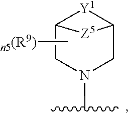

wherein Y¹ is independently selected from C(O)NR⁹d, O and NR¹⁷; Z⁵ is independently selected from: CH₂, CH₂CH₂, CH₂—O—CH₂CH₂, CH₂—O—CH₂, CH₂—NR¹⁷—CH₂CH₂ and CH₂—NR¹⁷—CH₂; R¹⁷ is independently at each occurrence selected from H, C₁-C₄-haloalkyl, and C₁-C₄-alkyl; Rod is independently selected from H and C₁-C₄-alkyl; and n5 is an integer selected from 0, 1, 2, 3 and 4. For the absence of doubt, throughout this specification, the n5 R⁹ groups may be attached to either ring of the bridged bicyclic ring system. Z⁵ is independently selected from: CH₂, CH₂CH₂, CH₂—O—CH₂CH₂, CH₂—O—CH₂. Y¹ may be independently selected from O and NR¹⁷. Y¹ may be NR¹⁷. Y¹ may be NH. n5 may be 0. R⁹ may independently at each occurrence be methyl.

It may be that R¹ and R^{5a} together with the nitrogen to which they are attached form a ring system having the structure:

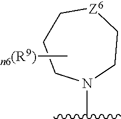

wherein Z⁶ is independently selected from C(O)NR^{9b}, O, S, S(O)₂, S(O), S(O)(NR^{9b}), S(O)(NH) and NR^{9b}; R^{9b} is independently at each occurrence selected from H and C₁-C₄-alkyl; and n6 is an integer selected from 0, 1, 2, 3 and 4. n6 may be 0. Z⁶ may be selected from NR^{9b}, O, S, S(O)₂, S(O), S(O)(NR^{9b}), and S(O)(NH). Z⁶ may be selected from C(O)NR^{9b}, O, S, S(O)₂, S(O), S(O)(NR^{9b}), and S(O)(NH). Z⁶ may be selected from C(O)NR^{9b}, O, S, S(O)₂, S(O) and S(O)(NH). Z⁶ may be selected from O, S, S(O)₂, S(O) and S(O)(NH). Z⁶ may be selected from NR^{9b}, O and S. Z⁶ may be selected from O and S. Z⁶ may be O.

It may be that R¹ and R^{5a} together with the nitrogen to which they are attached form a ring system having the structure:

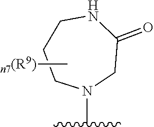

wherein n7 is an integer selected from 0, 1, 2 and 3.

It may be that R¹ and R^{5a} together with the nitrogen to which they are attached form a ring system having the structure:

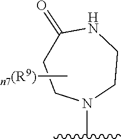

wherein n7 is an integer selected from 0, 1, 2 and 3.

It may be that R¹ and R^{5a} together with the nitrogen to which they are attached form a ring system having the structure:

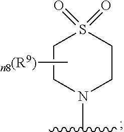

wherein n8 is an integer selected from 0, 1, 2 and 3.

n7 may be 0.

It may be that R¹ and R^{5a} together with the nitrogen to which they are attached form a ring system having the structure:

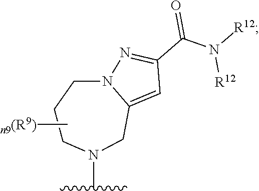

Wherein R¹² is independently at each occurrence selected from H, C₁-C₄-haloalkyl, and C₁-C₄-alkyl; and n9 is an integer selected from 0, 1, 2 and 3. R¹² may be C₁-C₄-alkyl e.g., methyl. n9 may be 0.

It may be that, where R¹ and R^{5a} together with the nitrogen to which they are attached form a ring system, e.g. one of the ring systems described above, that ring system is substituted with at least one hydroxy group.

It may be that R¹ and R^{5a} together with the nitrogen to which they are attached form a ring system having the structure:

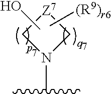

wherein Z⁷ is independently selected from carbon, C(O)NR^{9b}, O, S, S(O)₂, S(O), S(O)(NR^{9b}) and S(O)(NH); p7 is selected from 2 and 3, q2 is 2; and r6 is selected from 0, 1 and 2. Z⁷ may be selected from carbon, O, S, S(O)₂, S(O). Z⁷ may be carbon. Z⁷ may be selected from O and S. Z⁷ may be O. p7 may be 2. p7 may be 3. r6 may be 0. r6 may be 1. Where present, R⁹ may be C₁-C₄-alkyl, e.g. methyl.

It may be that R¹ and R^{5a} together with the nitrogen to which they are attached form a ring system having the structure:

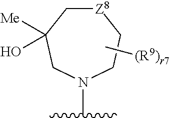

wherein Z⁸ is independently selected from a bond, carbon, C(O)NR^{9b}, O, S, S(O)₂, S(O), S(O)(NR^{9b}) and S(O)(NH); and r7 is selected from 0, 1 and 2. Z⁸ may be a bond. Z⁸ may be carbon. Z⁸ may be selected from O and S. Z⁸ may be O. r7 may be 0.

It may be that R¹ and R^{5a} together with the nitrogen to which they are attached form a ring system having the structure:

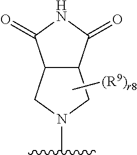

wherein r8 is an integer selected from 0, 1 and 2.

It may be that R¹ and R^{5a} together with the nitrogen to which they are attached form a ring system having the structure:

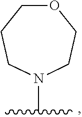 [SMILES: *N1CCCOCC1]

R² may be C₀-C₄-alkylene-R^{2a}. R² may be CH₂—R^{2a}. R^{2a} may be selected from monocyclic 4- to 7-membered heterocycloalkyl group, a fused, spirofused or bridged bicyclic 6- to 11-membered heterocycloalkyl group; wherein said R^{2a} group is optionally substituted with from 1 to 6 R¹⁰ groups. R^{2a} may comprise at least one nitrogen in the ring system. R^{2a} may comprise a single nitrogen in the ring system. R^{2a} may be selected from monocyclic 4- to 7-membered heterocycloalkyl group, a fused, spirofused or bridged bicyclic 6- to 11-membered heterocycloalkyl group; wherein said R^{2a} group is optionally substituted with from 1 to 6 R¹⁰ groups and wherein R^{2a} comprises at least one nitrogen in the ring system. R^{2a} may be monocyclic 4- to 7-membered heterocycloalkyl group; wherein said R^{2a} group is optionally substituted with from 1 to 6 R¹⁰ groups and wherein R^{2a} comprises at least one nitrogen in the ring system. R^{2a} may be a fused, spirofused or bridged bicyclic 6- to 11-membered heterocycloalkyl group; wherein said R^{2a} group is optionally substituted with from 1 to 6 R¹⁰ groups and wherein R^{2a} comprises at least one nitrogen in the ring system.

R² may have the structure:

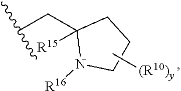

wherein R¹⁵ is independently selected from H, C₁-C₄-alkyl; wherein R¹⁶ is independently selected from H, C₁-C₄-alkyl and cyclopropyl; or wherein R¹⁵ and R¹⁶ together with the atoms to which they are attached form a 5- or 6-membered heterocycloalkyl ring, optionally substituted with 1 or 2 R¹⁰ groups; and y is independently selected from 0, 1, 2, 3, and 4. y may be selected from 0 and 1. y may be 0. y may be 1. R¹⁵ may be H. R¹⁶ may be C₁-C₄-alkyl.

R² may have the structure:

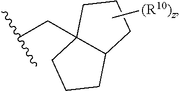

wherein z is independently selected from 0, 1, 2, 3, and 4. z may be selected from 0 and 1. z may be 0. z may be 1.

R⁴ may be phenyl, said phenyl being optionally fused to a C₅-C₇-cycloalkyl ring, wherein R⁴ is optionally substituted with from 1 to 4 R¹⁴ groups. R⁴ may be phenyl, optionally substituted with from 1 to 4 R¹⁴ groups. R⁴ may be monocyclic 4- to 7-membered cycloalkyl. R⁴ may be monocyclic 4- to 7-membered heterocycloalkyl, said heterocycloalkyl being optionally fused to a C₅-C₆-heteroaryl ring. R⁴ may be a 5-membered heterocycloalkyl, e.g., thiophenyl, fused to a monocyclic 5- to 6-membered cycloalkyl, e.g., cyclohexyl. R⁴ may be substituted with from 1 to 4 R¹⁴ groups. For the avoidance of doubt, where R⁴ comprises a fused ring structure, e.g. a 5-membered heterocycloalkyl fused to a monocyclic 5- to 6-membered cycloalkyl, the 1 to 4 R¹⁴ groups may be attached to either ring. For example, the 5-membered heterocycloalkyl may be substituted with e.g., cyano and —NH₂ and the monocyclic 5- to 6-membered cycloalkyl to which it is fused may be further substituted with e.g., C₁-C₄-alkyl.

R⁴ may have the structure:

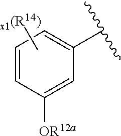

wherein R^{12a} is independently H or C₁-C₄-alkyl; x1 is independently selected from 0, 1, 2 and 3. R^{12a} may be H.

R^{3a} may be selected from halo, C₁-C₄-alkyl, O—C₁-C₄-alkyl, C₁-C₄-haloalkyl, O—C₁-C₄-haloalkyl, cyclopropyl, nitro and cyano. R^{3a} may be selected from H, halo and C₁-C₄-alkyl. R^{3a} may be F. R^{3a} may be C₁-C₄-alkyl, e.g. Me. R^{3a} may be H.

R^{3b} may be selected from halo, C₁-C₄-alkyl, O—C₁-C₄-alkyl, C₁-C₄-haloalkyl, O—C₁-C₄-haloalkyl, cyclopropyl, nitro and cyano. R^{3b} may be F. R^{3b} may be C₁-C₄-alkyl, e.g. Me. R^{3b} may be H.

R^{3c} may be selected from H, halo and C₁-C₄-alkyl. R^{3c} may be selected from H, F, Cl and CH₃. R^{3c} may be F. R^{3c} may be C₁-C₄-alkyl, e.g. Me. R^{3c} may be H.

R⁴ may be naphthyl, optionally substituted with from 1 to 4 R¹⁴ groups. R⁴ may have the structure:

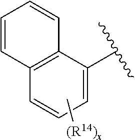

wherein x is independently selected from 0, 1, 2, 3, and 4. For the absence of doubt, throughout this specification, the x R¹⁴ groups may be attached to either ring of the naphthyl group. R¹⁴ may be independently selected from OR¹², halo e.g., F, and C₁-C₄-alkyl e.g., ethyl. x may be 0. x may be 1. x may be 2. x may be 3.

R⁴ may have the structure:

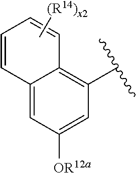

wherein R^{12a} is independently H or C₁-C₄-alkyl; x2 is independently selected from 0, 1, 2 and 3. For the absence of doubt, throughout this specification, the x2 R¹⁴ groups may be attached to either ring of the naphthyl group. R^{12a} may be H.

R⁴ may have the structure:

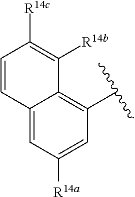

wherein R^{14a} is OR¹² e.g., OH; R^{14b} is C₁-C₄-alkyl e.g., methyl or ethyl; and R^{14c} is halo e.g, F.

R⁴ may have the structure:

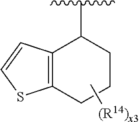

wherein R¹⁴ is independently H, cyano, NR¹²R¹³ and C₁-C₄-alkyl. X³ is independently selected from 0, 1, 2 and 3. For the absence of doubt, throughout this specification, the x3 R¹⁴ groups may be attached to either ring of the heterocyclyl group.

R⁴ may be 5-, 6-, 9- or 10-membered monocyclic or bicyclic heteroaryl, optionally substituted with from 1 to 4 R¹⁴ groups. R⁴ may be 9- or 10-membered bicyclic heteroaryl, optionally substituted with from 1 to 4 R¹⁴ groups.

R^{5a} may be H. R^{5a} may be C₁-C₄-alkyl, e.g. methyl.

R^{5b} may be H. R^{5b} may be C₁-C₄-alkyl, e.g. methyl.

R⁶ may be selected from C₁-C₄-haloalkyl, C₁-C₄-alkyl and C₃-C₄-cycloalkyl. R⁶ may be C₁-C₄-alkyl. R⁶ may be C₁-C₂-alkyl, e.g. methyl.

R⁷ may be selected from H and C₁-C₄-alkyl. R⁷ may be H. R⁷ may be C₁-C₄-alkyl, e.g. methyl.

R⁸ may be selected from H and C₁-C₄-alkyl. R⁸ may be H. R⁸ may be C₁-C₄-alkyl, e.g. methyl.

R⁹ may be independently at each occurrence selected from oxo, fluoro, cyano, NR¹²R¹³, OR¹², COR¹², C₁-C₄-alkyl, CONR¹²R¹³, C₁-C₄-alkyl substituted with NR¹²R¹³, C₁-C₄-alkyl substituted with OR¹², C₁-C₄-alkyl substituted with cyano, C₁-C₄-alkyl substituted with phenyl. R⁹ may be independently at each occurrence selected from oxo, fluoro, NR¹²R¹³, OR¹², COR¹², CONR¹²R¹³; C₁-C₄-alkyl, C₁-C₄-alkyl substituted with NR¹²R¹³, C₁-C₄-alkyl substituted with phenyl and C₁-C₄-alkyl substituted with OR 12.

R⁹ may be independently at each occurrence selected from oxo, fluoro, cyano, NR¹²R¹³, OR¹², C₁-C₄-alkyl, C₁-C₄-alkyl substituted with NR¹²R¹³, C₁-C₄-alkyl substituted with OR¹², C₁-C₄-alkyl substituted with cyano. R⁹ may be independently at each occurrence selected from oxo, fluoro, NR¹²R¹³, OR¹², C₁-C₄-alkyl, C₁-C₄-alkyl substituted with NR¹²R¹³ and C₁-C₄-alkyl substituted with OR¹².

R⁹ may be independently at each occurrence selected from oxo, halo, cyano, NR¹²R¹³ provided that R¹² is not H and R¹³ is not H, OR¹², COR¹², CO₂R¹², CONR¹²R¹², C₁-C₄-alkyl, C₁-C₄-alkyl substituted with NR¹²R¹³ provided that R¹² is not H and R¹³ is not H, C₁-C₄-alkyl substituted with OR¹², C₁-C₄-alkyl substituted with cyano, C₂-C₄-alkenyl, C₂-C₄-alkynyl, C₁-C₄-haloalkyl and cyclopropyl.

R⁹ may be independently at each occurrence selected from oxo, halo, cyano, NR¹²R¹³ provided that R¹² is not H and R¹³ is not H, OR¹², CO₂R¹², CONR¹²R¹², C₁-C₄-alkyl, C₁-C₄-alkyl substituted with NR¹²R¹³ provided that R¹² is not H and R¹³ is not H, C₁-C₄-alkyl substituted with OR¹², C₁-C₄-alkyl substituted with cyano, C₂-C₄-alkenyl, C₂-C₄-alkynyl, C₁-C₄-haloalkyl and cyclopropyl.

R⁹ may be independently at each occurrence selected from C₁-C₄-alkyl e.g., methyl and OR¹² e.g., OH.

R¹⁰ may be independently at each occurrence selected from oxo, halo, cyano, NR¹²R¹³, OR¹², CO₂R¹², CONR¹²R¹², C₁-C₄-alkyl, C₁-C₄-alkyl substituted with NR¹²R¹³, C₁-C₄-alkyl substituted with OR¹², C₁-C₄-alkyl substituted with cyano, C₁-C₄-alkyl substituted with phenyl, C₂-C₄-alkenyl, C₂-C₄-alkynyl, C₁-C₄-haloalkyl and cyclopropyl.

R¹⁰ may be independently at each occurrence selected from oxo, halo, cyano, NR¹²R¹³, OR¹², CO₂R¹², CONR¹²R¹², C₁-C₄-alkyl, C₁-C₄-alkyl substituted with NR¹²R¹³, C₁-C₄-alkyl substituted with OR¹², C₁-C₄-alkyl substituted with cyano, C₂-C₄-alkenyl, C₂-C₄-alkynyl, C₁-C₄-haloalkyl and cyclopropyl.

R¹⁰ may be independently at each occurrence selected from oxo, fluoro, NR¹²R¹³, OR¹², C₁-C₄-alkyl, C₁-C₄-alkyl substituted with phenyl, C₁-C₄-alkyl substituted with NR¹²R¹³ and C₁-C₄-alkyl substituted with OR¹².

R¹⁰ may be independently at each occurrence selected from oxo, fluoro, NR¹²R¹³, OR¹², C₁-C₄-alkyl, C₁-C₄-alkyl substituted with NR¹²R¹³ and C₁-C₄-alkyl substituted with OR 12.

R^{10a} may be independently at each occurrence selected from oxo, halo, cyano, NR¹²R¹³, OR¹², CO₂R¹², CONR¹²R¹², C₁-C₄-alkyl, C₁-C₄-alkyl substituted with NR¹²R^{13a} C₁-C₄-alkyl substituted with OR¹², C₁-C₄-alkyl substituted with cyano, C₁-C₄-alkyl substituted with phenyl, C₂-C₄-alkenyl, C₂-C₄-alkynyl, C₁-C₄-haloalkyl and cyclopropyl.

R^{10a} may be independently at each occurrence selected from oxo, halo, cyano, NR¹²R¹³, OR¹², CO₂R¹², CONR¹²R¹², C₁-C₄-alkyl, C₁-C₄-alkyl substituted with NR¹²R^{13a}, C₁-C₄-alkyl substituted with OR¹², C₁-C₄-alkyl substituted with cyano, C₂-C₄-alkenyl, C₂-C₄-alkynyl, C₁-C₄-haloalkyl and cyclopropyl.

R^{10a} may be independently at each occurrence selected from oxo, fluoro, NR¹²R^{13a} OR¹², C₁-C₄-alkyl, C₁-C₄-alkyl substituted with phenyl, C₁-C₄-alkyl substituted with NR¹²R^{13a} and C₁-C₄-alkyl substituted with OR¹².

R^{10a} may be independently at each occurrence selected from oxo, fluoro, NR¹²R^{13a}, OR¹², C₁-C₄-alkyl, C₁-C₄-alkyl substituted with NR¹²R^{13a} and C₁-C₄-alkyl substituted with OR 12.

R^{10a} may be independently at each occurrence selected from halo, cyano, C₁-C₄-alkyl, C₁-C₄-haloalkyl and cyclopropyl. R^{10a} may be C₁-C₄-haloalkyl e.g., trifluoromethyl.

R¹¹ may be each independently at each occurrence selected from halo, cyano, nitro, NR¹²R¹³, OR¹², C₁-C₄-alkyl, C₁-C₄-alkyl substituted with NR¹²R¹³, C₁-C₄-alkyl substituted with OR¹², monocyclic 4- to 7-membered cycloalkyl or heterocycloalkyl, C₁-C₄-haloalkyl and cyclopropyl. R¹¹ may be each independently at each occurrence selected from OR¹², monocyclic 4- to 7-membered cycloalkyl or heterocycloalkyl, C₁-C₄-alkyl, C₁-C₄-haloalkyl and cyclopropyl.

R¹¹ may be each independently at each occurrence selected from halo, cyano, nitro, NR¹²R¹³, OR¹², C₁-C₄-alkyl, C₁-C₄-alkyl substituted with NR¹²R¹³, C₁-C₄-alkyl substituted with OR¹², C₁-C₄-haloalkyl and cyclopropyl. R¹¹ may be each independently at each occurrence selected from OR¹², C₁-C₄-alkyl, C₁-C₄-haloalkyl and cyclopropyl.

It may be that R¹² is independently selected at each occurrence from H, C₁-C₄-haloalkyl, C₁-C₄-alkyl and cyclopropyl; and R¹³ is independently at each occurrence selected from H, C₁-C₄-alkyl, C₁-C₄-haloalkyl and C(O)—C₁-C₄-alkyl.

It may be that R¹² is independently selected at each occurrence from H, C₁-C₄-haloalkyl, and C₁-C₄-alkyl; and R¹³ is independently at each occurrence selected from H, C₁-C₄-alkyl, C₁-C₄-haloalkyl and C(O)—C₁-C₄-alkyl.

R¹² may independently at each occurrence be selected from H, cyclopropyl and C₁-C₄-alkyl. R¹² may be C₁-C₄-alkyl e.g., methyl. R¹² may be cyclopropyl.

R¹² may independently at each occurrence be selected from H and C₁-C₄-alkyl.

R¹³ may independently at each occurrence be selected from H and C₁-C₄-alkyl.

In compounds of formula (I), it may be that R¹² and R¹³ together with the nitrogen to which they are attached form a ring system selected from: monocyclic 4- to 7-membered heterocycloalkyl group, a fused, spirofused or bridged bicyclic 6- to 11-membered heterocycloalkyl group, said heterocycloalkyl group being optionally substituted with from 1 to 6 R¹⁰ groups. It may be that R¹² and R¹³ together with the nitrogen to which they are attached form a monocyclic 4- to 7-membered heterocycloalkyl group e.g., piperidinyl.

It may be that R¹² and R¹³ together with the nitrogen to which they are attached form a ring system selected from: monocyclic 4- to 7-membered heterocycloalkyl group, a fused, spirofused or bridged bicyclic 6- to 11-membered heterocycloalkyl group, said heterocycloalkyl group being optionally substituted with from 1 to 6 R^{10a} groups. It may be that R¹² and R¹³ together with the nitrogen to which they are attached form a monocyclic 4- to 7-membered heterocycloalkyl group e.g., piperidinyl.

R^{13a} may be independently at each occurrence selected from H, C₁-C₄-alkyl and C(O)—C₁-C₄-alkyl. R^{13a} may be independently at each occurrence selected from H and C₁-C₄-alkyl.

R¹⁴ may be each independently at each occurrence selected from halo, cyano, nitro, NR¹²R¹³, OR¹², C₁-C₄-alkyl, C₁-C₄-alkyl substituted with NR¹²R¹³, C₁-C₄-alkyl substituted with OR¹², C₁-C₄-haloalkyl, phenyl and cyclopropyl. R¹⁴ may be each independently at each occurrence selected from OR¹², C₁-C₄-alkyl, C₁-C₄-haloalkyl, phenyl and cyclopropyl.

R¹⁴ may be each independently at each occurrence selected from halo, cyano, nitro, NR¹²R¹³, OR¹², C₁-C₄-alkyl, C₁-C₄-alkyl substituted with NR¹²R¹³, C₁-C₄-alkyl substituted with OR¹², C₁-C₄-haloalkyl and cyclopropyl. R¹⁴ may be each independently at each occurrence selected from OR¹², C₁-C₄-alkyl, C₁-C₄-haloalkyl and cyclopropyl.

The compound of formula (I) may be selected from:

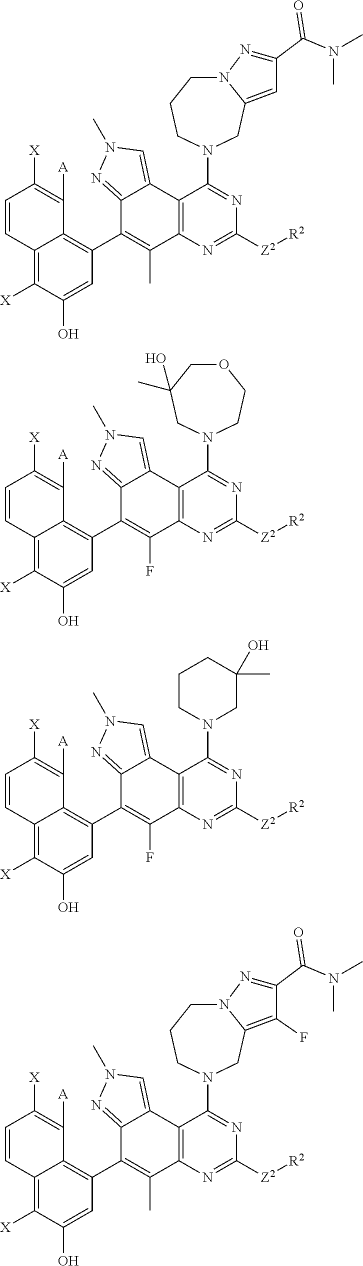

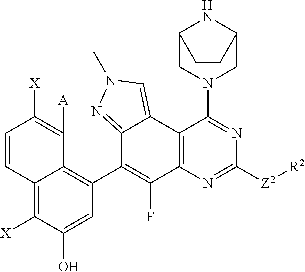

where A is methyl, ethyl, propyl, cyclopropyl, ethenyl or ethynyl, and X is F or Cl.

The compound of formula (I) may be selected from:

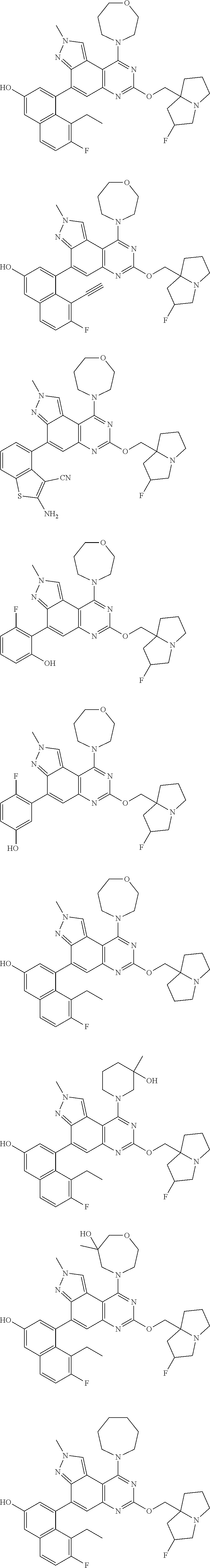

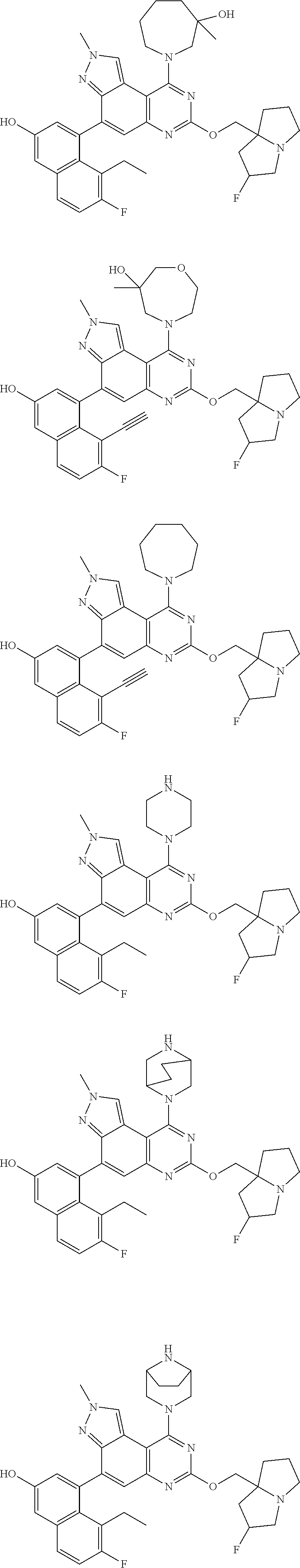 [SMILES: CCc1c(F)ccc2cc(O)cc(-c3cc4nc(OCC56CCCN5CC(F)C6)nc(N5CCCCC(C)(O)C5)c4c4cn(C)nc34)c12 ; C#Cc1c(F)ccc2cc(O)cc(-c3cc4nc(OCC56CCCN5CC(F)C6)nc(N5CCOCC(C)(O)C5)c4c4cn(C)nc34)c12 ; C#Cc1c(F)ccc2cc(O)cc(-c3cc4nc(OCC56CCCN5CC(F)C6)nc(N5CCCCCC5)c4c4cn(C)nc34)c12 ; CCc1c(F)ccc2cc(O)cc(-c3cc4nc(OCC56CCCN5CC(F)C6)nc(N5CCNCC5)c4c4cn(C)nc34)c12 ; CCc1c(F)ccc2cc(O)cc(-c3cc4nc(OCC56CCCN5CC(F)C6)nc(N5CC6CCC5CN6)c4c4cn(C)nc34)c12 ; CCc1c(F)ccc2cc(O)cc(-c3cc4nc(OCC56CCCN5CC(F)C6)nc(N5CC6CCC(C5)N6)c4c4cn(C)nc34)c12]

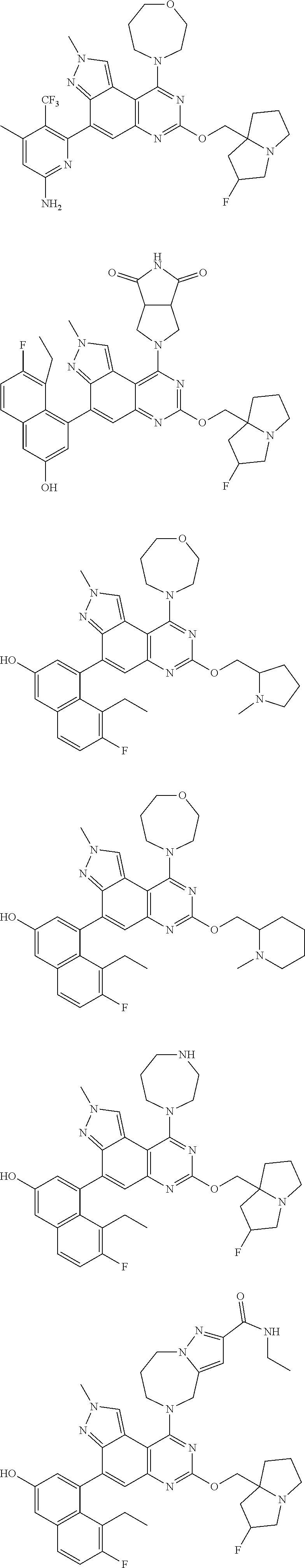 [SMILES: Cc1cc(N)nc(-c2cc3nc(OCC45CCCN4CC(F)C5)nc(N4CCCOCC4)c3c3cn(C)nc23)c1C(F)(F)F ; CCc1c(F)ccc2cc(O)cc(-c3cc4nc(OCC56CCCN5CC(F)C6)nc(N5CC6C(=O)NC(=O)C6C5)c4c4cn(C)nc34)c12 ; CCc1c(F)ccc2cc(O)cc(-c3cc4nc(OCC5CCCN5C)nc(N5CCCOCC5)c4c4cn(C)nc34)c12 ; CCc1c(F)ccc2cc(O)cc(-c3cc4nc(OCC5CCCCN5C)nc(N5CCCOCC5)c4c4cn(C)nc34)c12 ; CCc1c(F)ccc2cc(O)cc(-c3cc4nc(OCC56CCCN5CC(F)C6)nc(N5CCCNCC5)c4c4cn(C)nc34)c12 ; CCNC(=O)c1cc2n(n1)CCCN(c1nc(OCC34CCCN3CC(F)C4)nc3cc(-c4cc(O)cc5ccc(F)c(CC)c45)c4nn(C)cc4c13)C2]

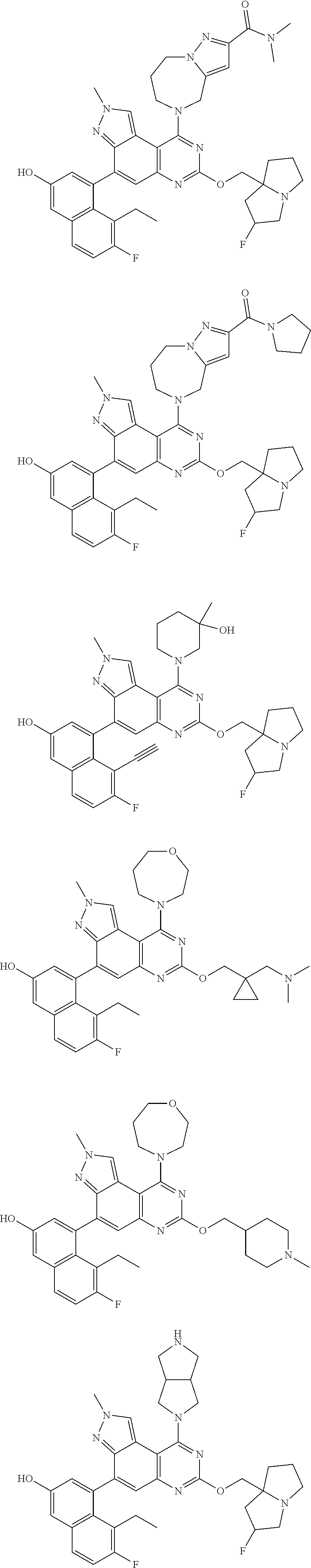

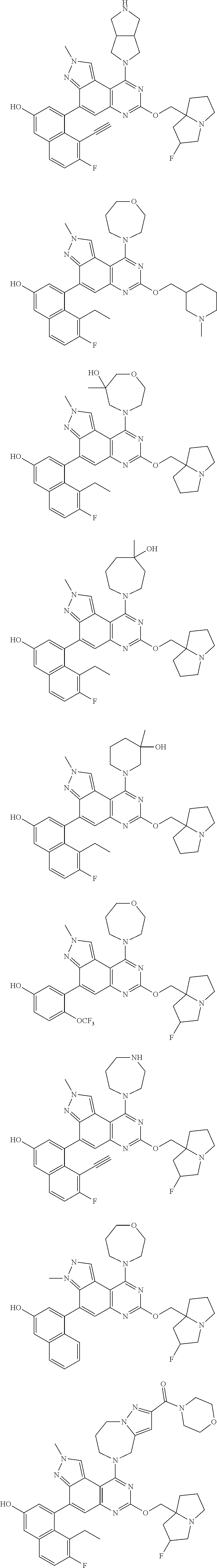

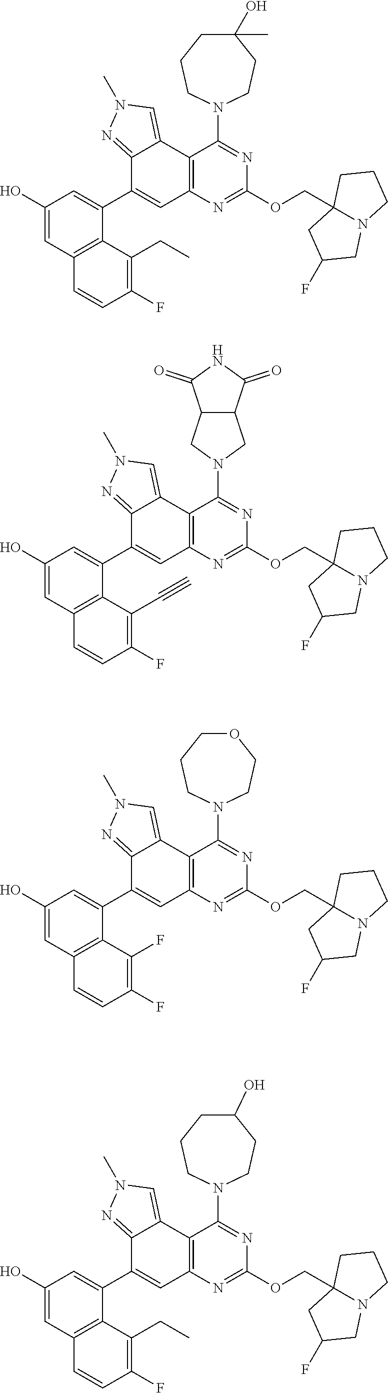 [SMILES: CCc1c(F)ccc2cc(O)cc(-c3cc4nc(OCC56CCCN5CC(F)C6)nc(N5CCCC(C)(O)CC5)c4c4cn(C)nc34)c12 ; C#Cc1c(F)ccc2cc(O)cc(-c3cc4nc(OCC56CCCN5CC(F)C6)nc(N5CC6C(=O)NC(=O)C6C5)c4c4cn(C)nc34)c12 ; Cn1cc2c(n1)c(-c1cc(O)cc3ccc(F)c(F)c13)cc1nc(OCC34CCCN3CC(F)C4)nc(N3CCCOCC3)c12 ; CCc1c(F)ccc2cc(O)cc(-c3cc4nc(OCC56CCCN5CC(F)C6)nc(N5CCCC(O)CC5)c4c4cn(C)nc34)c12]

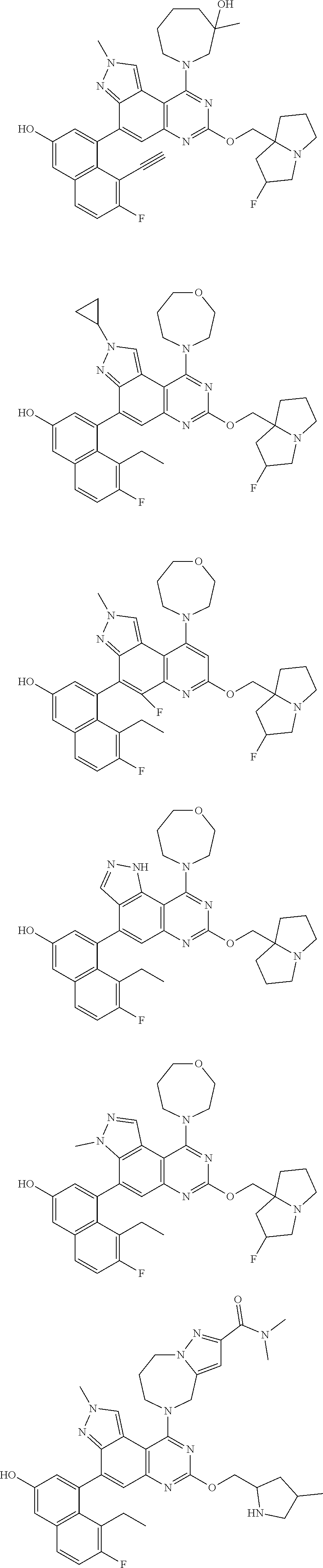 [SMILES: C#Cc1c(F)ccc2cc(O)cc(-c3cc4nc(OCC56CCCN5CC(F)C6)nc(N5CCCCC(C)(O)C5)c4c4cn(C)nc34)c12 ; CCc1c(F)ccc2cc(O)cc(-c3cc4nc(OCC56CCCN5CC(F)C6)nc(N5CCCOCC5)c4c4cn(C5CC5)nc34)c12 ; CCc1c(F)ccc2cc(O)cc(-c3c(F)c4nc(OCC56CCCN5CC(F)C6)cc(N5CCCOCC5)c4c4cn(C)nc34)c12 ; CCc1c(F)ccc2cc(O)cc(-c3cc4nc(OCC56CCCN5CCC6)nc(N5CCCOCC5)c4c4[nH]ncc34)c12 ; CCc1c(F)ccc2cc(O)cc(-c3cc4nc(OCC56CCCN5CC(F)C6)nc(N5CCCOCC5)c4c4cnn(C)c34)c12 ; CCc1c(F)ccc2cc(O)cc(-c3cc4nc(OCC5CC(C)CN5)nc(N5CCCn6nc(C(=O)N(C)C)cc6C5)c4c4cn(C)nc34)c12]

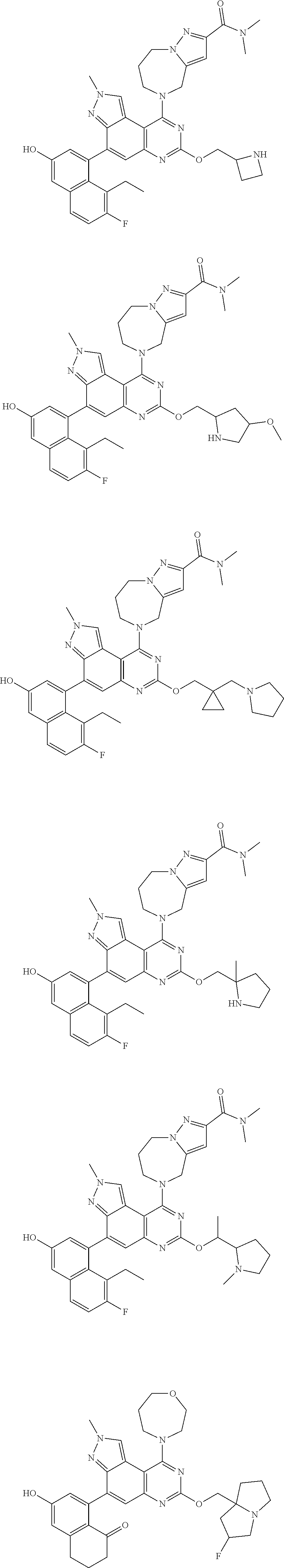

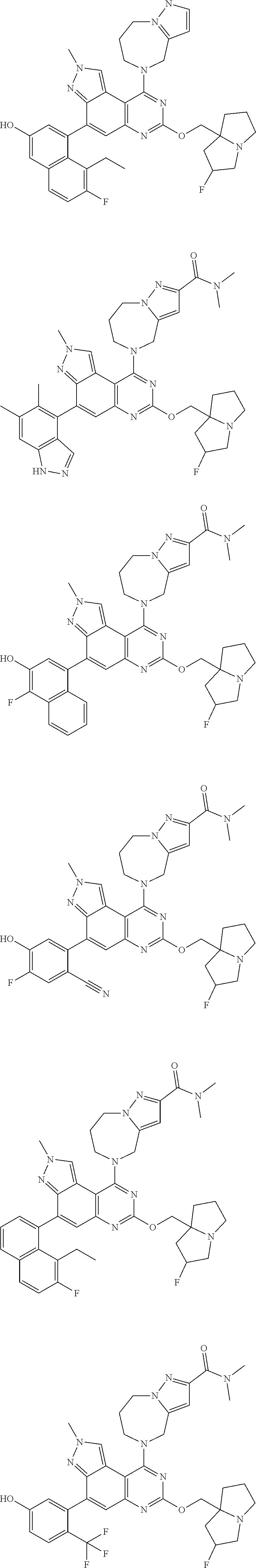

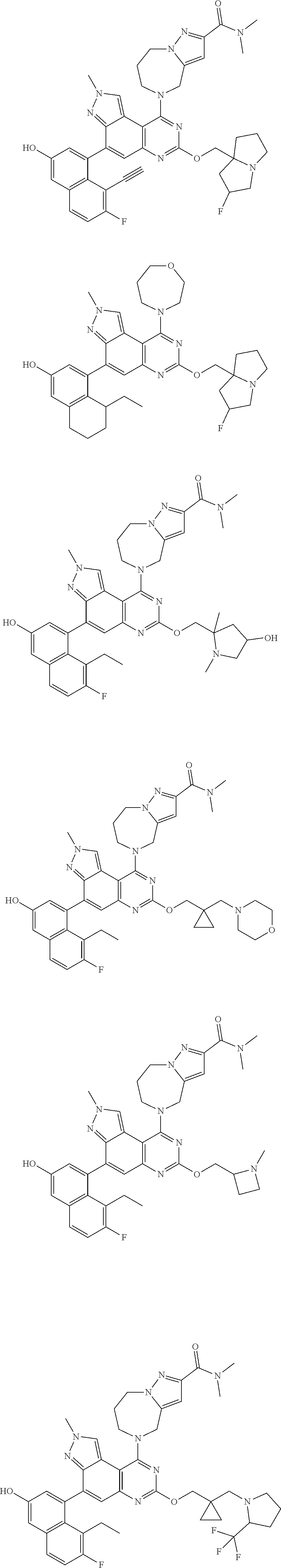

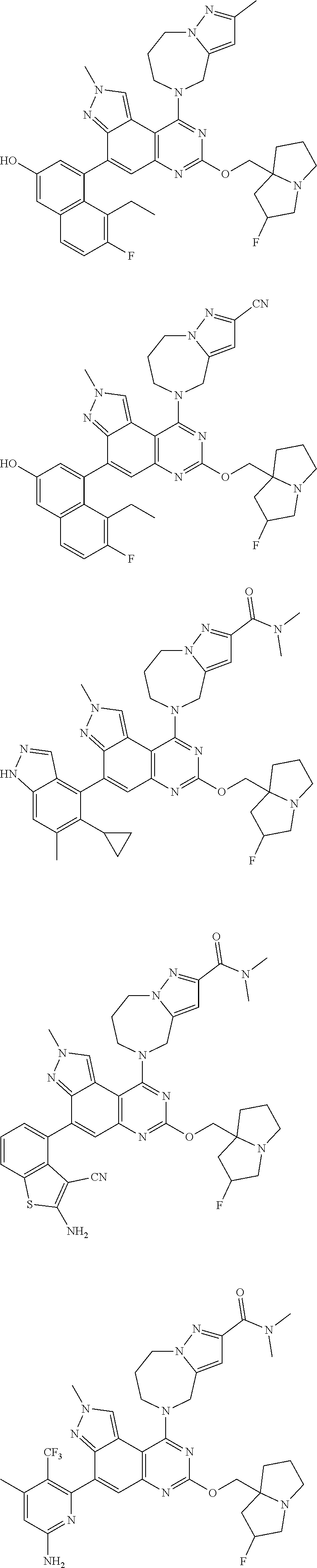 [SMILES: CCc1c(F)ccc2cc(O)cc(-c3cc4nc(OCC56CCCN5CC(F)C6)nc(N5CCCn6nc(C)cc6C5)c4c4cn(C)nc34)c12 ; CCc1c(F)ccc2cc(O)cc(-c3cc4nc(OCC56CCCN5CC(F)C6)nc(N5CCCn6nc(C#N)cc6C5)c4c4cn(C)nc34)c12 ; Cc1cc2[nH]ncc2c(-c2cc3nc(OCC45CCCN4CC(F)C5)nc(N4CCCn5nc(C(=O)N(C)C)cc5C4)c3c3cn(C)nc23)c1C1CC1 ; CN(C)C(=O)c1cc2n(n1)CCCN(c1nc(OCC34CCCN3CC(F)C4)nc3cc(-c4cccc5sc(N)c(C#N)c45)c4nn(C)cc4c13)C2 ; Cc1cc(N)nc(-c2cc3nc(OCC45CCCN4CC(F)C5)nc(N4CCCn5nc(C(=O)N(C)C)cc5C4)c3c3cn(C)nc23)c1C(F)(F)F]

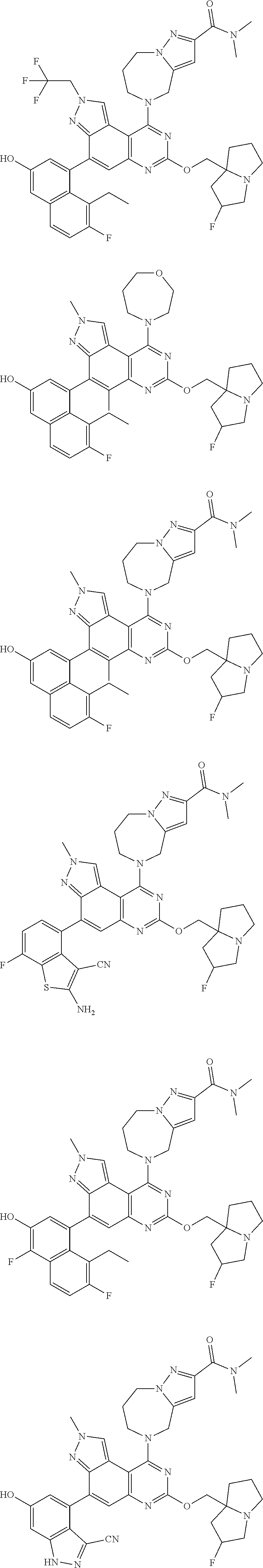

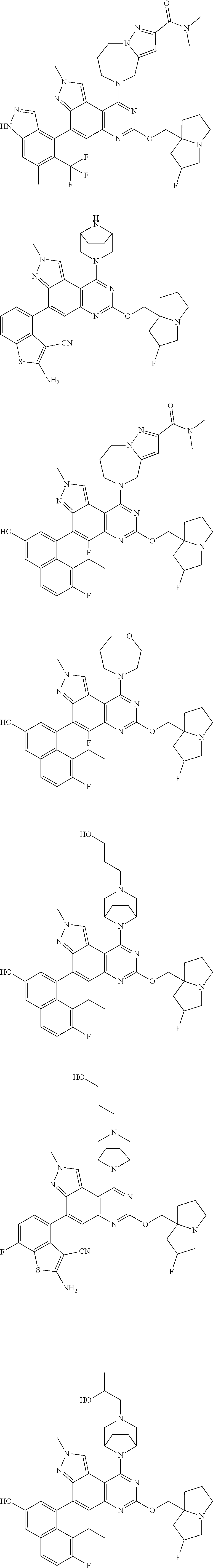

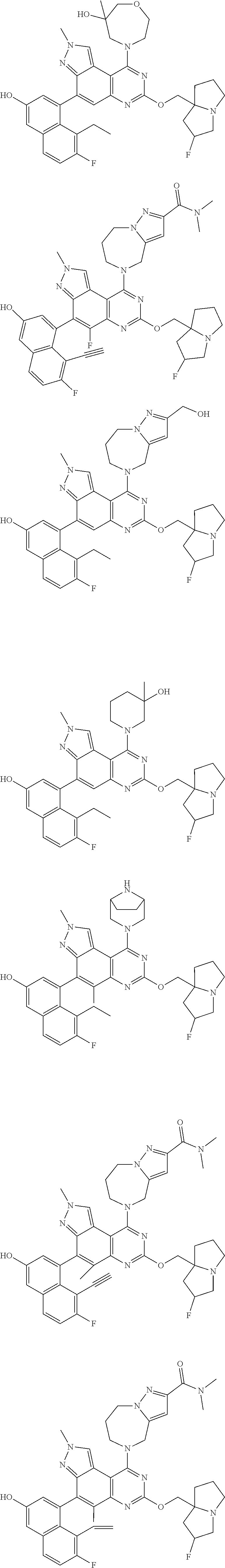

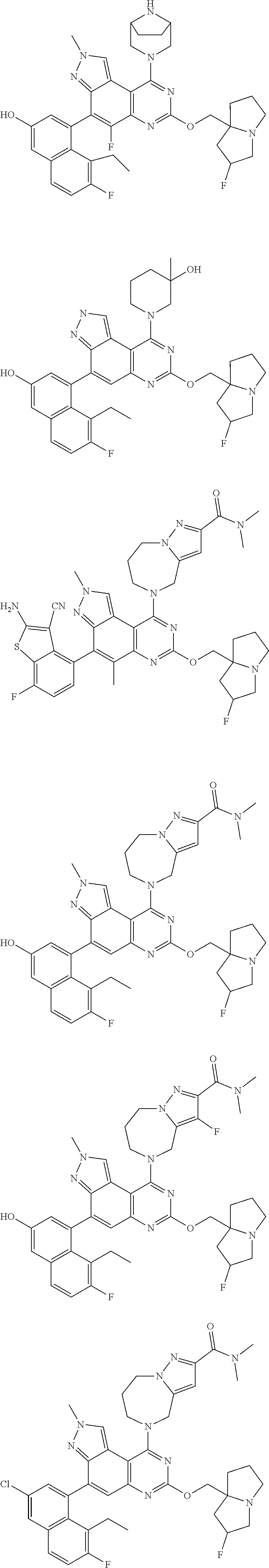

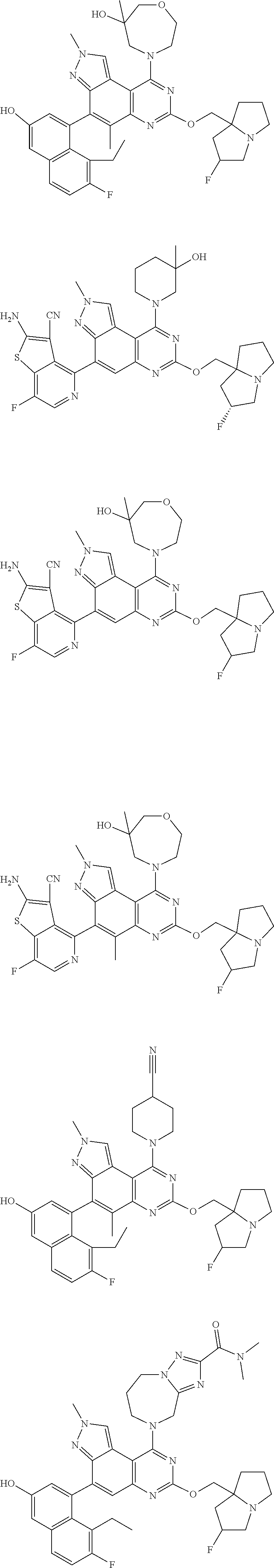

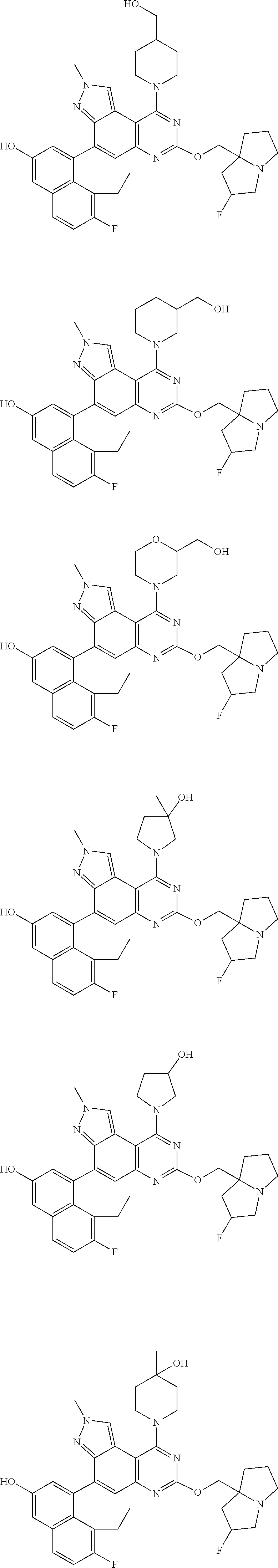 [SMILES: CCc1c(F)ccc2cc(O)cc(-c3cc4nc(OCC56CCCN5CC(F)C6)nc(N5CCC(CO)CC5)c4c4cn(C)nc34)c12 ; CCc1c(F)ccc2cc(O)cc(-c3cc4nc(OCC56CCCN5CC(F)C6)nc(N5CCCC(CO)C5)c4c4cn(C)nc34)c12 ; CCc1c(F)ccc2cc(O)cc(-c3cc4nc(OCC56CCCN5CC(F)C6)nc(N5CCOC(CO)C5)c4c4cn(C)nc34)c12 ; CCc1c(F)ccc2cc(O)cc(-c3cc4nc(OCC56CCCN5CC(F)C6)nc(N5CCC(C)(O)C5)c4c4cn(C)nc34)c12 ; CCc1c(F)ccc2cc(O)cc(-c3cc4nc(OCC56CCCN5CC(F)C6)nc(N5CCC(O)C5)c4c4cn(C)nc34)c12 ; CCc1c(F)ccc2cc(O)cc(-c3cc4nc(OCC56CCCN5CC(F)C6)nc(N5CCC(C)(O)CC5)c4c4cn(C)nc34)c12]

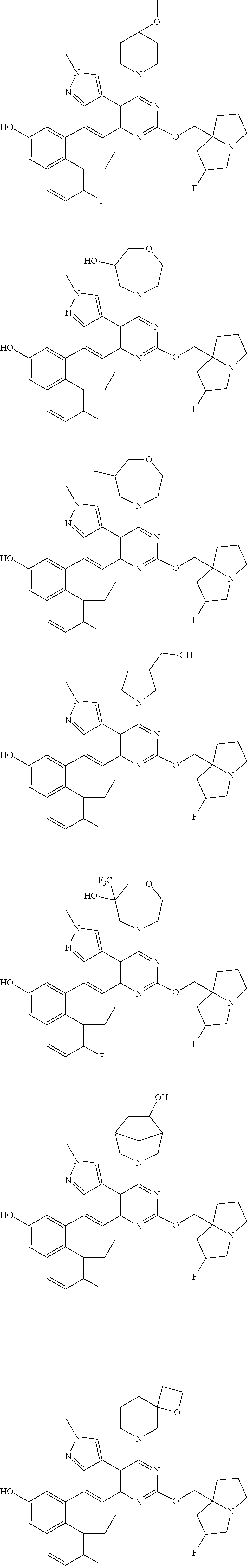

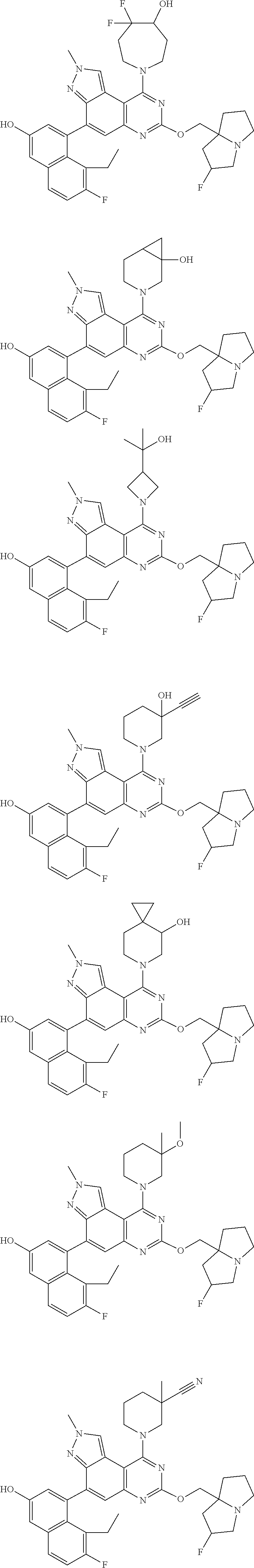

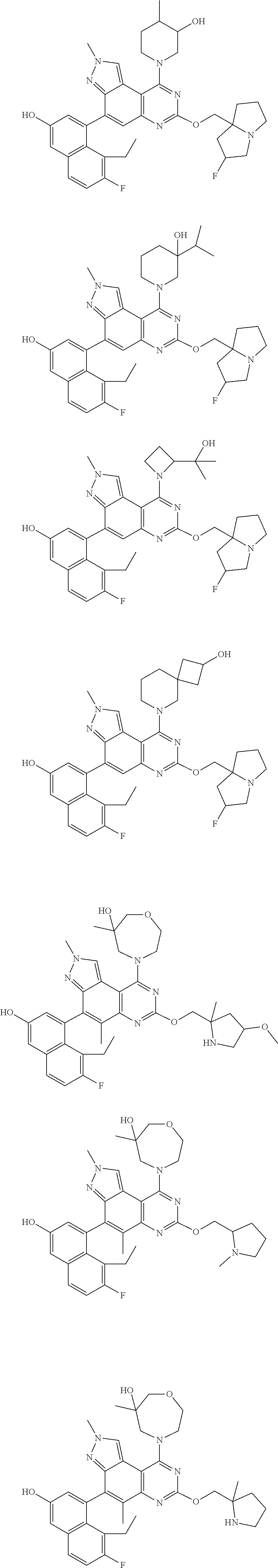

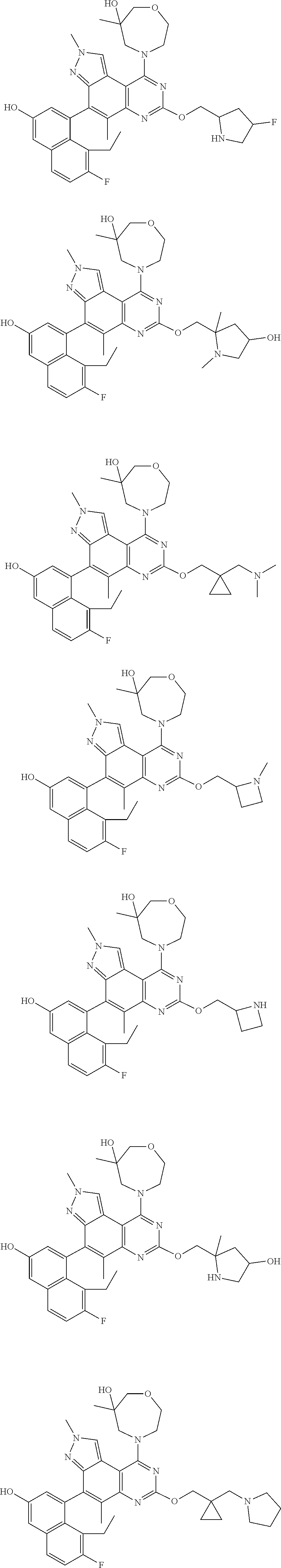

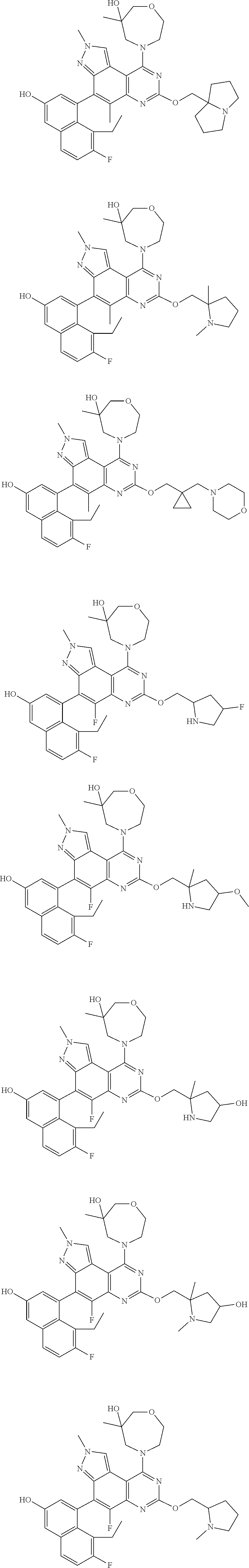

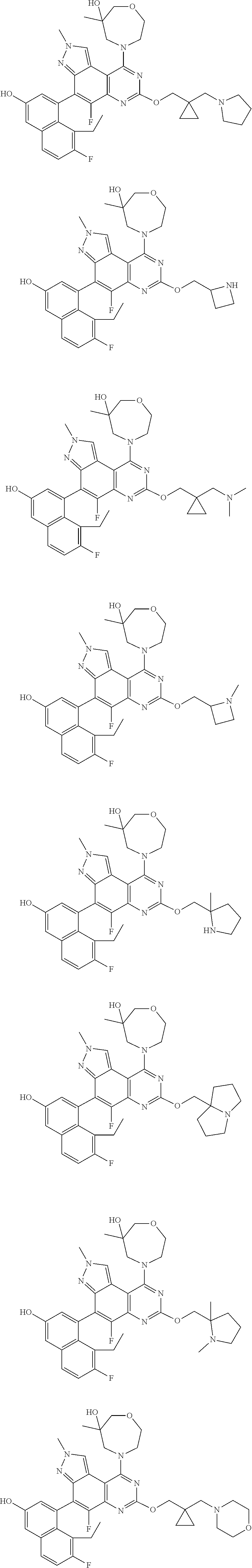

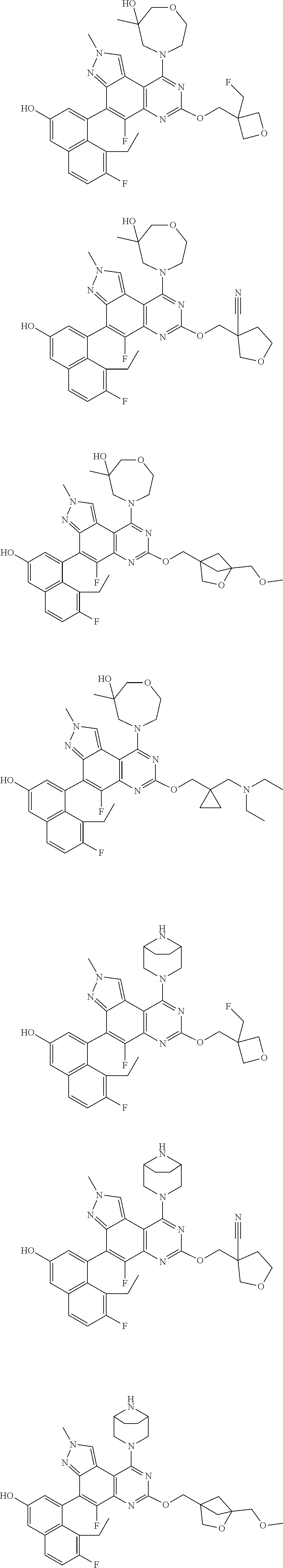

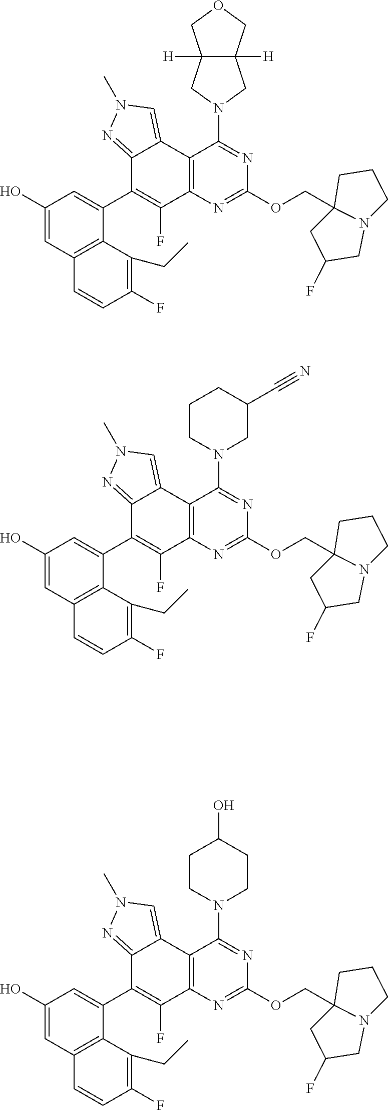 [SMILES: CCc1c(F)ccc2cc(O)cc(-c3c(F)c4nc(OCC56CCCN5CC(F)C6)nc(N5CC6COCC6C5)c4c4cn(C)nc34)c12 ; CCc1c(F)ccc2cc(O)cc(-c3c(F)c4nc(OCC56CCCN5CC(F)C6)nc(N5CCCC(C#N)C5)c4c4cn(C)nc34)c12 ; CCc1c(F)ccc2cc(O)cc(-c3c(F)c4nc(OCC56CCCN5CC(F)C6)nc(N5CCC(O)CC5)c4c4cn(C)nc34)c12]

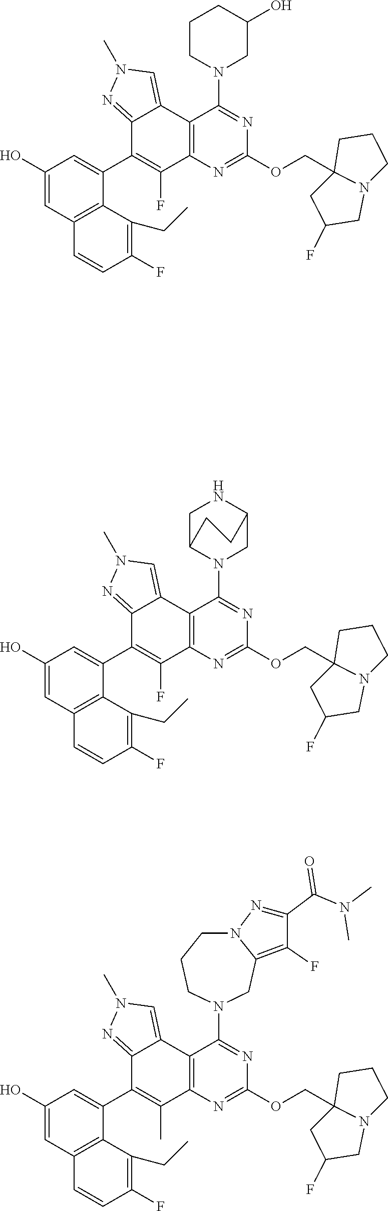 [SMILES: CCc1c(F)ccc2cc(O)cc(-c3c(F)c4nc(OCC56CCCN5CC(F)C6)nc(N5CCCC(O)C5)c4c4cn(C)nc34)c12 ; CCc1c(F)ccc2cc(O)cc(-c3c(F)c4nc(OCC56CCCN5CC(F)C6)nc(N5CC6CCC5CN6)c4c4cn(C)nc34)c12 ; CCc1c(F)ccc2cc(O)cc(-c3c(C)c4nc(OCC56CCCN5CC(F)C6)nc(N5CCCn6nc(C(=O)N(C)C)c(F)c6C5)c4c4cn(C)nc34)c12]

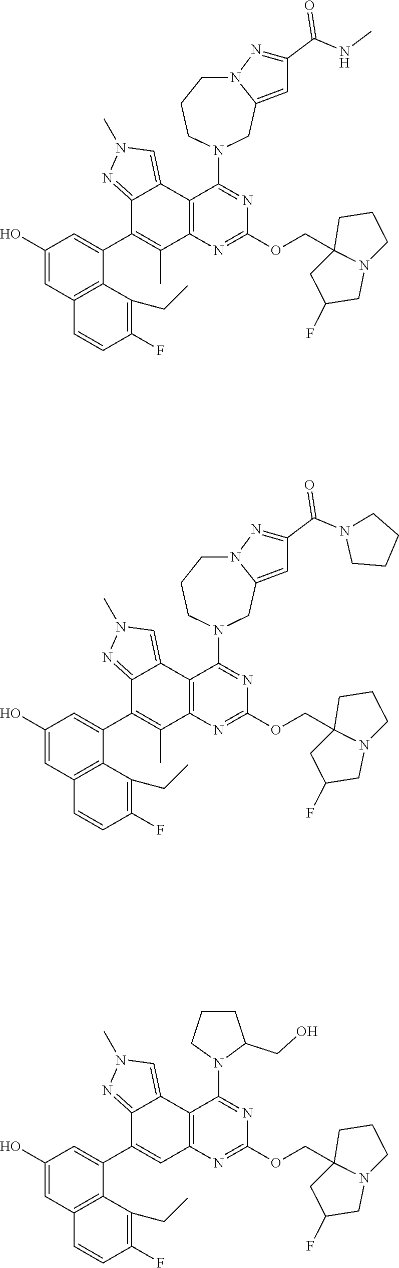 [SMILES: CCc1c(F)ccc2cc(O)cc(-c3c(C)c4nc(OCC56CCCN5CC(F)C6)nc(N5CCCn6nc(C(=O)NC)cc6C5)c4c4cn(C)nc34)c12 ; CCc1c(F)ccc2cc(O)cc(-c3c(C)c4nc(OCC56CCCN5CC(F)C6)nc(N5CCCn6nc(C(=O)N7CCCC7)cc6C5)c4c4cn(C)nc34)c12 ; CCc1c(F)ccc2cc(O)cc(-c3cc4nc(OCC56CCCN5CC(F)C6)nc(N5CCCC5CO)c4c4cn(C)nc34)c12]

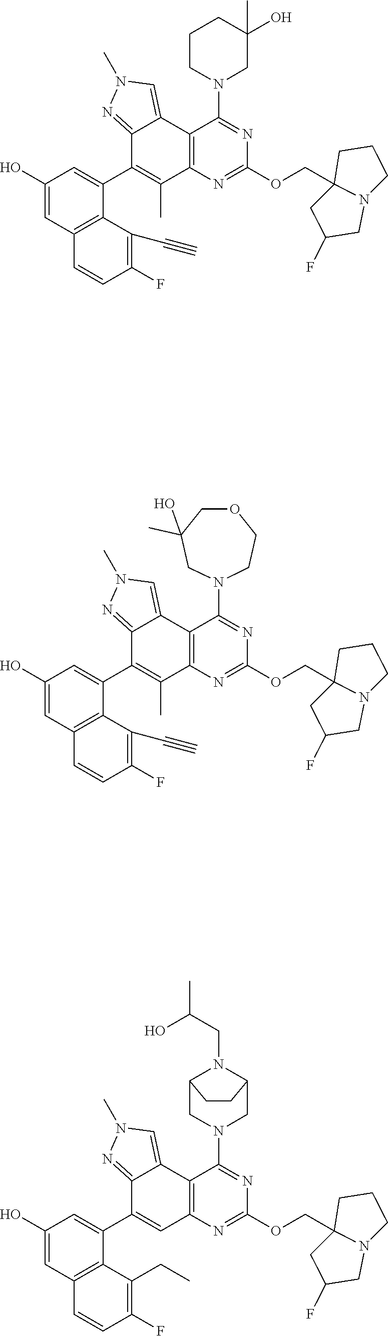 [SMILES: C#Cc1c(F)ccc2cc(O)cc(-c3c(C)c4nc(OCC56CCCN5CC(F)C6)nc(N5CCCC(C)(O)C5)c4c4cn(C)nc34)c12 ; C#Cc1c(F)ccc2cc(O)cc(-c3c(C)c4nc(OCC56CCCN5CC(F)C6)nc(N5CCOCC(C)(O)C5)c4c4cn(C)nc34)c12 ; CCc1c(F)ccc2cc(O)cc(-c3cc4nc(OCC56CCCN5CC(F)C6)nc(N5CC6CCC(C5)N6CC(C)O)c4c4cn(C)nc34)c12]

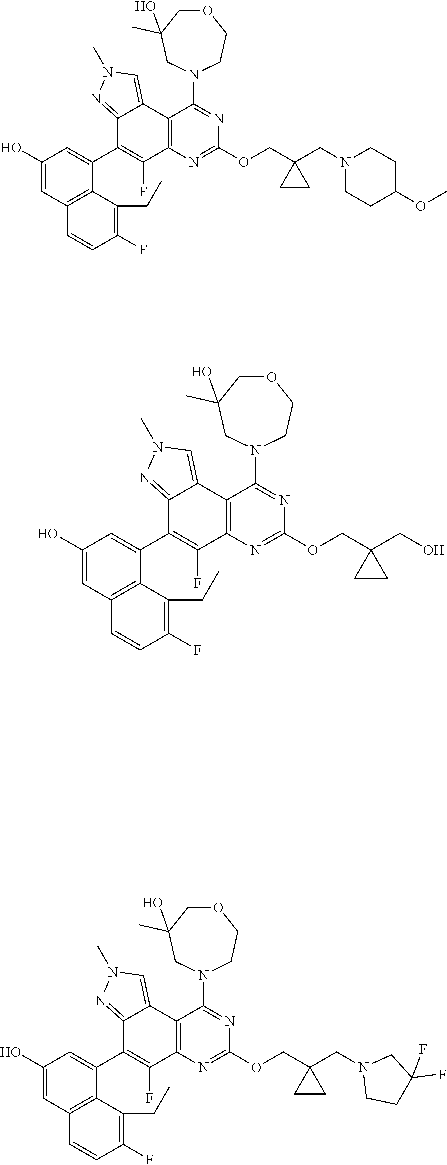 [SMILES: CCc1c(F)ccc2cc(O)cc(-c3c(F)c4nc(OCC5(CN6CCC(OC)CC6)CC5)nc(N5CCOCC(C)(O)C5)c4c4cn(C)nc34)c12 ; CCc1c(F)ccc2cc(O)cc(-c3c(F)c4nc(OCC5(CO)CC5)nc(N5CCOCC(C)(O)C5)c4c4cn(C)nc34)c12 ; CCc1c(F)ccc2cc(O)cc(-c3c(F)c4nc(OCC5(CN6CCC(F)(F)C6)CC5)nc(N5CCOCC(C)(O)C5)c4c4cn(C)nc34)c12]

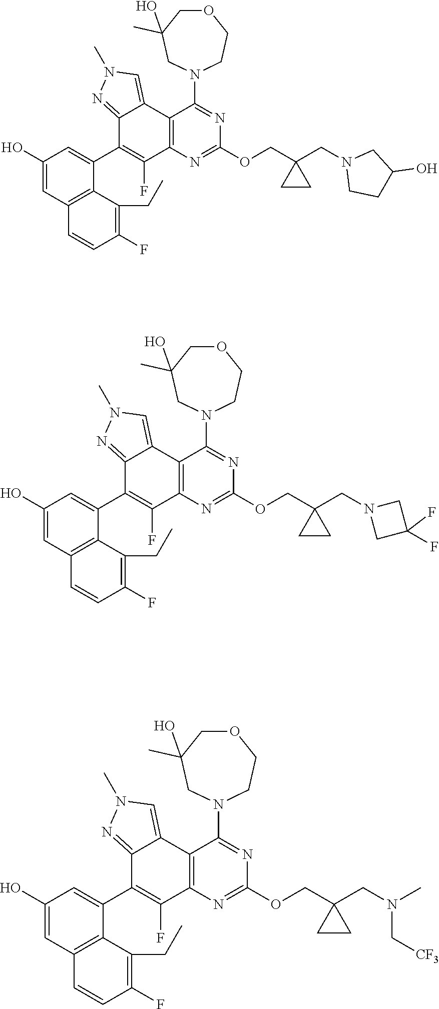 [SMILES: CCc1c(F)ccc2cc(O)cc(-c3c(F)c4nc(OCC5(CN6CCC(O)C6)CC5)nc(N5CCOCC(C)(O)C5)c4c4cn(C)nc34)c12 ; CCc1c(F)ccc2cc(O)cc(-c3c(F)c4nc(OCC5(CN6CC(F)(F)C6)CC5)nc(N5CCOCC(C)(O)C5)c4c4cn(C)nc34)c12 ; CCc1c(F)ccc2cc(O)cc(-c3c(F)c4nc(OCC5(CN(C)CC(F)(F)F)CC5)nc(N5CCOCC(C)(O)C5)c4c4cn(C)nc34)c12]

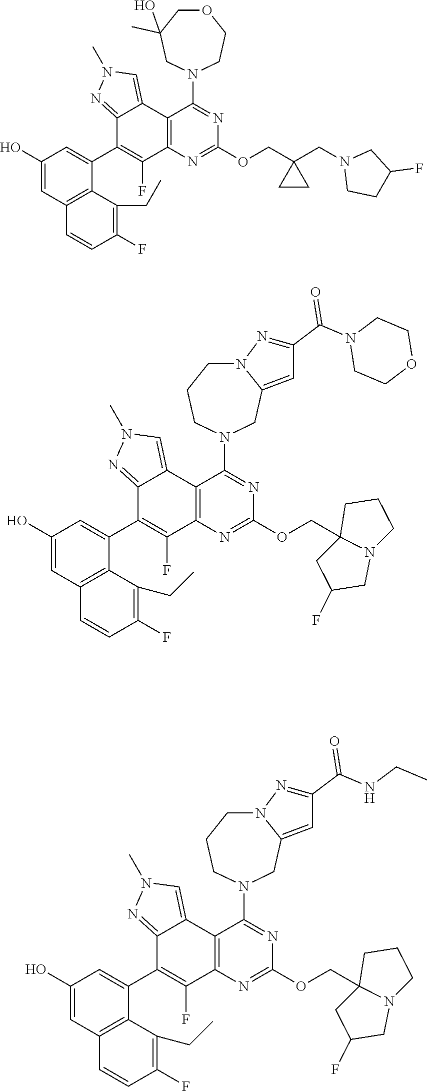 [SMILES: CCc1c(F)ccc2cc(O)cc(-c3c(F)c4nc(OCC5(CN6CCC(F)C6)CC5)nc(N5CCOCC(C)(O)C5)c4c4cn(C)nc34)c12 ; CCc1c(F)ccc2cc(O)cc(-c3c(F)c4nc(OCC56CCCN5CC(F)C6)nc(N5CCCn6nc(C(=O)N7CCOCC7)cc6C5)c4c4cn(C)nc34)c12 ; CCNC(=O)c1cc2n(n1)CCCN(c1nc(OCC34CCCN3CC(F)C4)nc3c(F)c(-c4cc(O)cc5ccc(F)c(CC)c45)c4nn(C)cc4c13)C2]

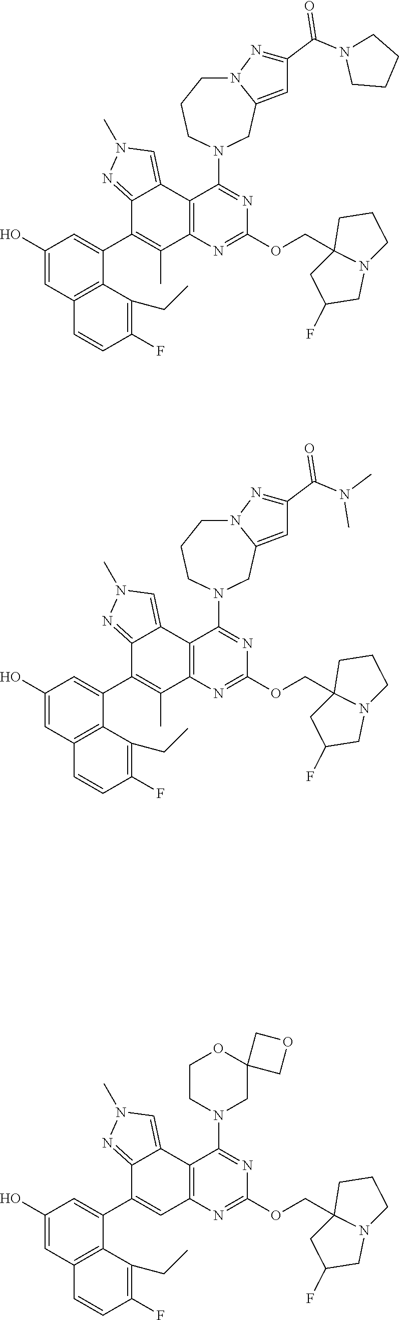 [SMILES: CCc1c(F)ccc2cc(O)cc(-c3c(C)c4nc(OCC56CCCN5CC(F)C6)nc(N5CCCn6nc(C(=O)N7CCCC7)cc6C5)c4c4cn(C)nc34)c12 ; CCc1c(F)ccc2cc(O)cc(-c3c(C)c4nc(OCC56CCCN5CC(F)C6)nc(N5CCCn6nc(C(=O)N(C)C)cc6C5)c4c4cn(C)nc34)c12 ; CCc1c(F)ccc2cc(O)cc(-c3cc4nc(OCC56CCCN5CC(F)C6)nc(N5CCOC6(COC6)C5)c4c4cn(C)nc34)c12]

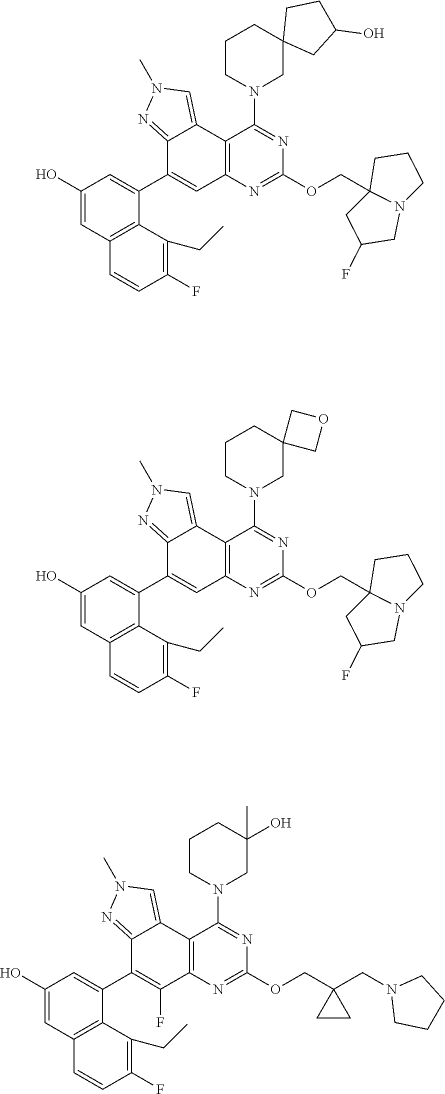 [SMILES: CCc1c(F)ccc2cc(O)cc(-c3cc4nc(OCC56CCCN5CC(F)C6)nc(N5CCCC6(CCC(O)C6)C5)c4c4cn(C)nc34)c12 ; CCc1c(F)ccc2cc(O)cc(-c3cc4nc(OCC56CCCN5CC(F)C6)nc(N5CCCC6(COC6)C5)c4c4cn(C)nc34)c12 ; CCc1c(F)ccc2cc(O)cc(-c3c(F)c4nc(OCC5(CN6CCCC6)CC5)nc(N5CCCC(C)(O)C5)c4c4cn(C)nc34)c12]

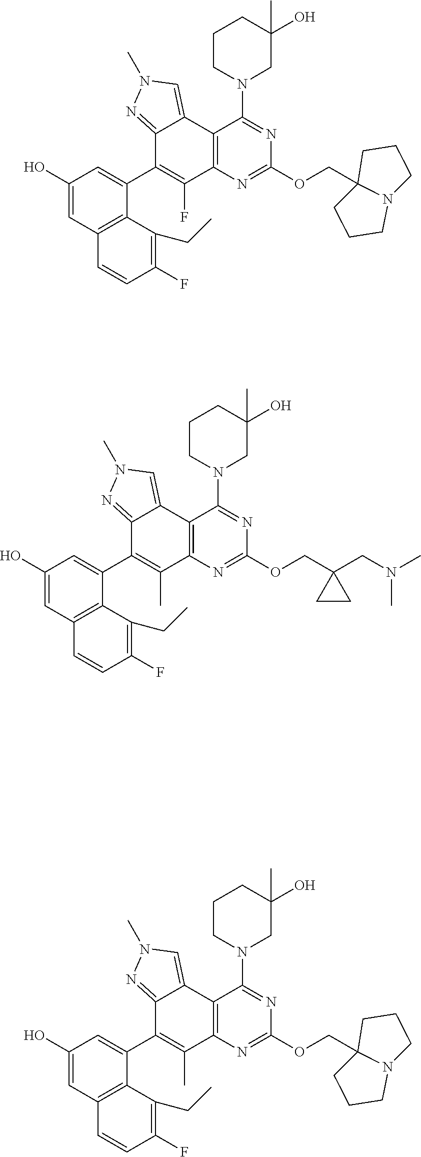 [SMILES: CCc1c(F)ccc2cc(O)cc(-c3c(F)c4nc(OCC56CCCN5CCC6)nc(N5CCCC(C)(O)C5)c4c4cn(C)nc34)c12 ; CCc1c(F)ccc2cc(O)cc(-c3c(C)c4nc(OCC5(CN(C)C)CC5)nc(N5CCCC(C)(O)C5)c4c4cn(C)nc34)c12 ; CCc1c(F)ccc2cc(O)cc(-c3c(C)c4nc(OCC56CCCN5CCC6)nc(N5CCCC(C)(O)C5)c4c4cn(C)nc34)c12]

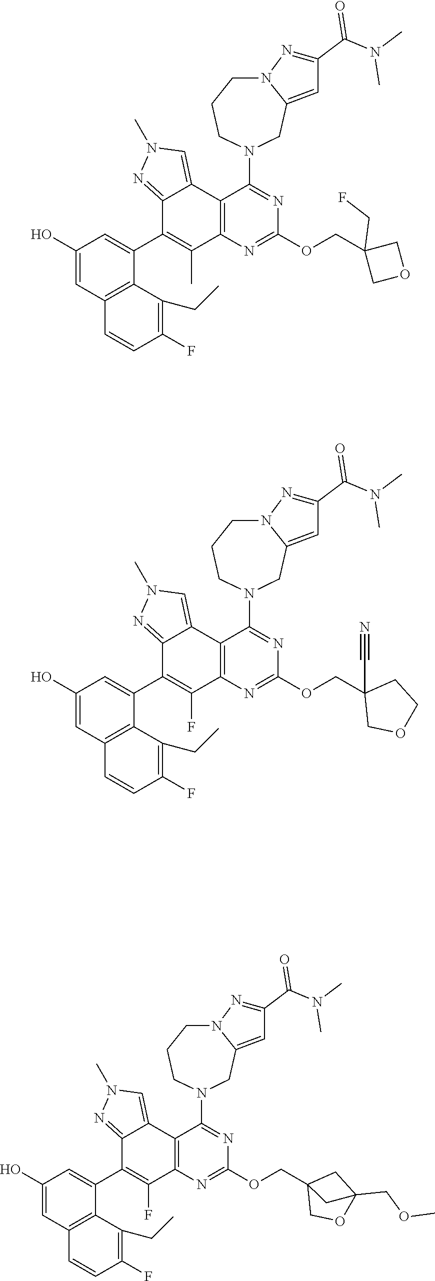 [SMILES: CCc1c(F)ccc2cc(O)cc(-c3c(C)c4nc(OCC5(CF)COC5)nc(N5CCCn6nc(C(=O)N(C)C)cc6C5)c4c4cn(C)nc34)c12 ; CCc1c(F)ccc2cc(O)cc(-c3c(F)c4nc(OCC5(C#N)CCOC5)nc(N5CCCn6nc(C(=O)N(C)C)cc6C5)c4c4cn(C)nc34)c12 ; CCc1c(F)ccc2cc(O)cc(-c3c(F)c4nc(OCC56COC(COC)(C5)C6)nc(N5CCCn6nc(C(=O)N(C)C)cc6C5)c4c4cn(C)nc34)c12]

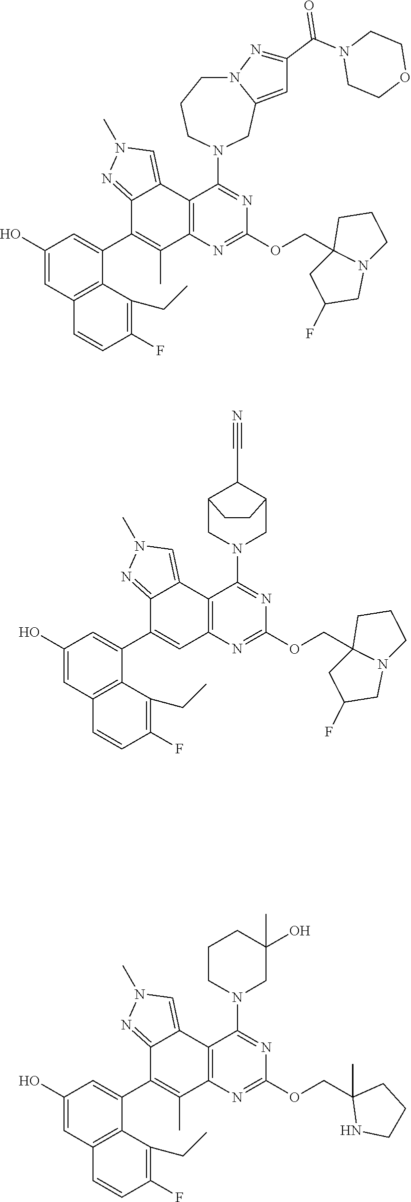 [SMILES: CCc1c(F)ccc2cc(O)cc(-c3c(C)c4nc(OCC56CCCN5CC(F)C6)nc(N5CCCn6nc(C(=O)N7CCOCC7)cc6C5)c4c4cn(C)nc34)c12 ; CCc1c(F)ccc2cc(O)cc(-c3cc4nc(OCC56CCCN5CC(F)C6)nc(N5CC6CCC(C5)C6C#N)c4c4cn(C)nc34)c12 ; CCc1c(F)ccc2cc(O)cc(-c3c(C)c4nc(OCC5(C)CCCN5)nc(N5CCCC(C)(O)C5)c4c4cn(C)nc34)c12]

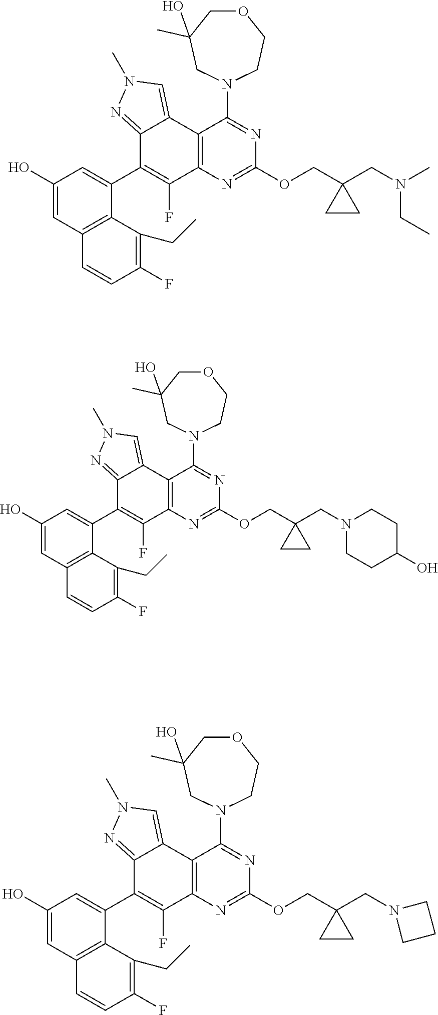 [SMILES: CCc1c(F)ccc2cc(O)cc(-c3c(F)c4nc(OCC5(CN(C)CC)CC5)nc(N5CCOCC(C)(O)C5)c4c4cn(C)nc34)c12 ; CCc1c(F)ccc2cc(O)cc(-c3c(F)c4nc(OCC5(CN6CCC(O)CC6)CC5)nc(N5CCOCC(C)(O)C5)c4c4cn(C)nc34)c12 ; CCc1c(F)ccc2cc(O)cc(-c3c(F)c4nc(OCC5(CN6CCC6)CC5)nc(N5CCOCC(C)(O)C5)c4c4cn(C)nc34)c12]

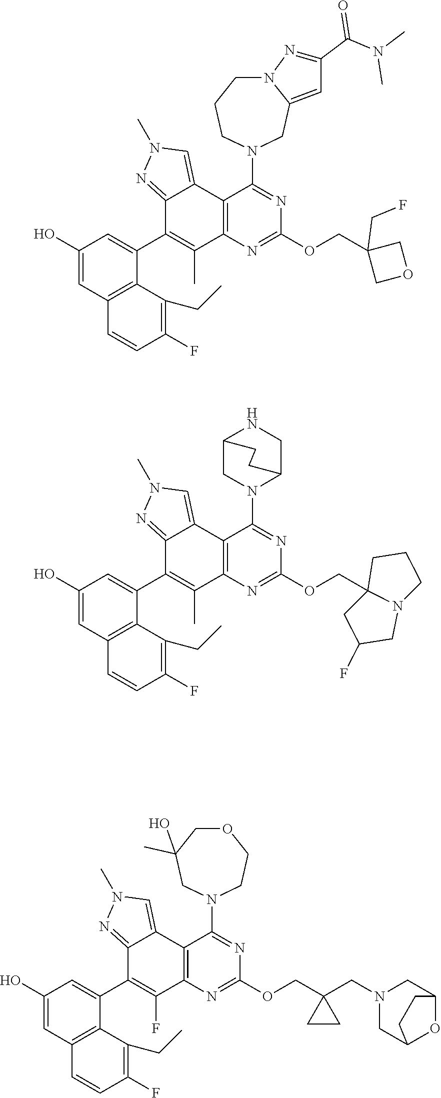 [SMILES: CCc1c(F)ccc2cc(O)cc(-c3c(C)c4nc(OCC5(CF)COC5)nc(N5CCCn6nc(C(=O)N(C)C)cc6C5)c4c4cn(C)nc34)c12 ; CCc1c(F)ccc2cc(O)cc(-c3c(C)c4nc(OCC56CCCN5CC(F)C6)nc(N5CC6CCC5CN6)c4c4cn(C)nc34)c12 ; CCc1c(F)ccc2cc(O)cc(-c3c(F)c4nc(OCC5(CN6CC7CCC(C6)O7)CC5)nc(N5CCOCC(C)(O)C5)c4c4cn(C)nc34)c12]

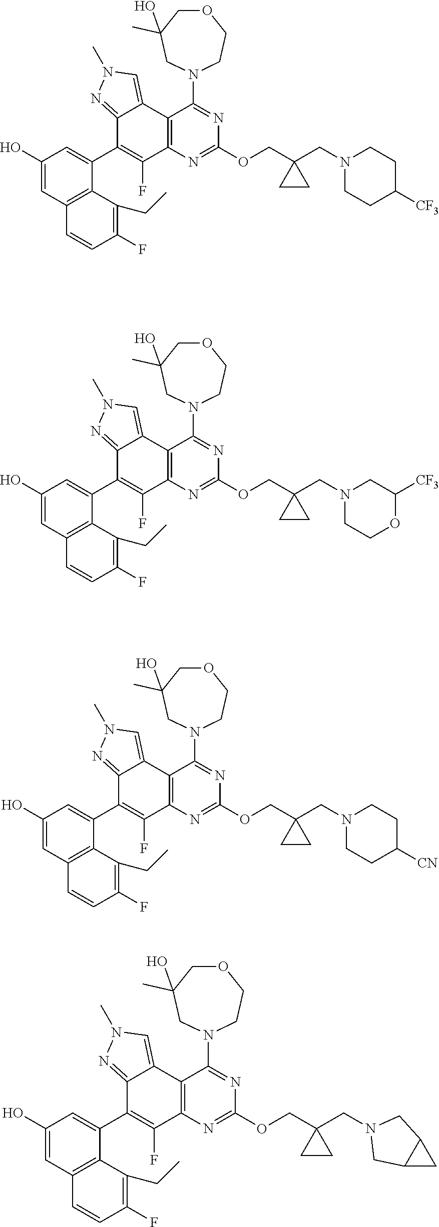 [SMILES: CCc1c(F)ccc2cc(O)cc(-c3c(F)c4nc(OCC5(CN6CCC(C(F)(F)F)CC6)CC5)nc(N5CCOCC(C)(O)C5)c4c4cn(C)nc34)c12 ; CCc1c(F)ccc2cc(O)cc(-c3c(F)c4nc(OCC5(CN6CCOC(C(F)(F)F)C6)CC5)nc(N5CCOCC(C)(O)C5)c4c4cn(C)nc34)c12 ; CCc1c(F)ccc2cc(O)cc(-c3c(F)c4nc(OCC5(CN6CCC(C#N)CC6)CC5)nc(N5CCOCC(C)(O)C5)c4c4cn(C)nc34)c12 ; CCc1c(F)ccc2cc(O)cc(-c3c(F)c4nc(OCC5(CN6CC7CC7C6)CC5)nc(N5CCOCC(C)(O)C5)c4c4cn(C)nc34)c12]

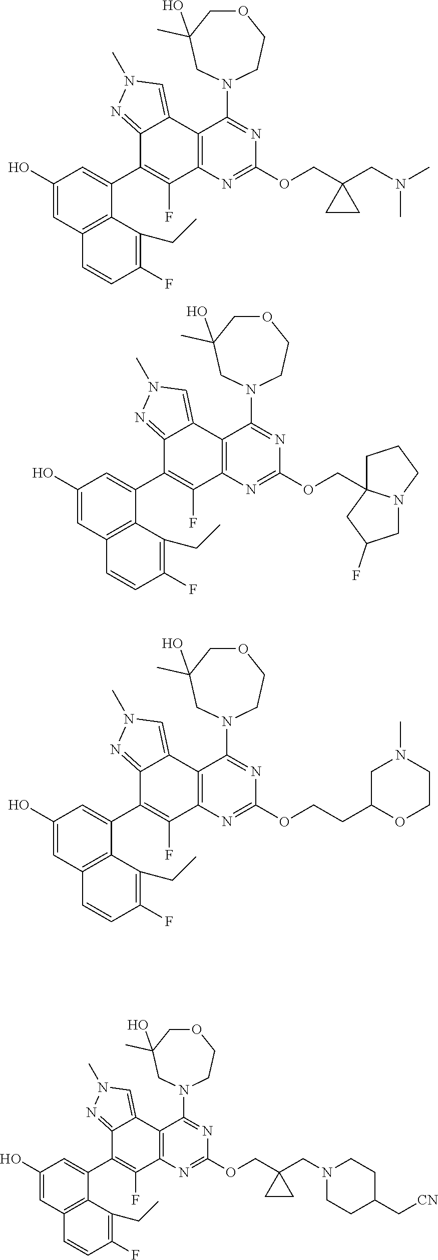

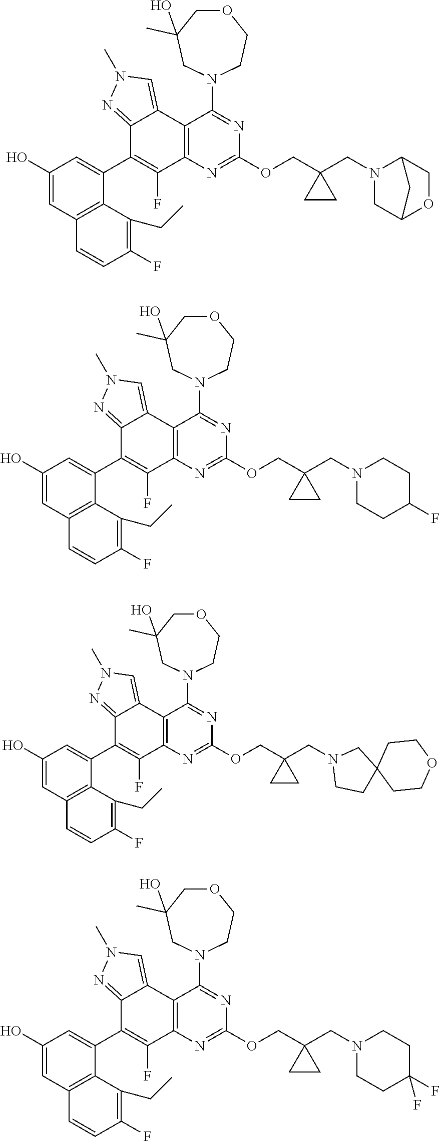 [SMILES: CCc1c(F)ccc2cc(O)cc(-c3c(F)c4nc(OCC5(CN6CC7CC6CO7)CC5)nc(N5CCOCC(C)(O)C5)c4c4cn(C)nc34)c12 ; CCc1c(F)ccc2cc(O)cc(-c3c(F)c4nc(OCC5(CN6CCC(F)CC6)CC5)nc(N5CCOCC(C)(O)C5)c4c4cn(C)nc34)c12 ; CCc1c(F)ccc2cc(O)cc(-c3c(F)c4nc(OCC5(CN6CCC7(CCOCC7)C6)CC5)nc(N5CCOCC(C)(O)C5)c4c4cn(C)nc34)c12 ; CCc1c(F)ccc2cc(O)cc(-c3c(F)c4nc(OCC5(CN6CCC(F)(F)CC6)CC5)nc(N5CCOCC(C)(O)C5)c4c4cn(C)nc34)c12]

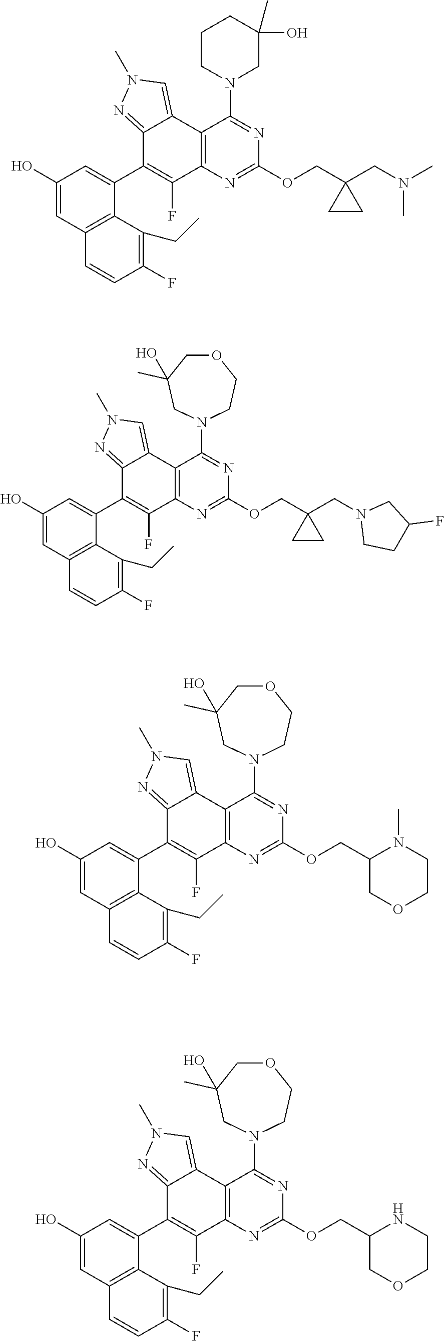 [SMILES: CCc1c(F)ccc2cc(O)cc(-c3c(F)c4nc(OCC5(CN(C)C)CC5)nc(N5CCCC(C)(O)C5)c4c4cn(C)nc34)c12 ; CCc1c(F)ccc2cc(O)cc(-c3c(F)c4nc(OCC5(CN6CCC(F)C6)CC5)nc(N5CCOCC(C)(O)C5)c4c4cn(C)nc34)c12 ; CCc1c(F)ccc2cc(O)cc(-c3c(F)c4nc(OCC5COCCN5C)nc(N5CCOCC(C)(O)C5)c4c4cn(C)nc34)c12 ; CCc1c(F)ccc2cc(O)cc(-c3c(F)c4nc(OCC5COCCN5)nc(N5CCOCC(C)(O)C5)c4c4cn(C)nc34)c12]

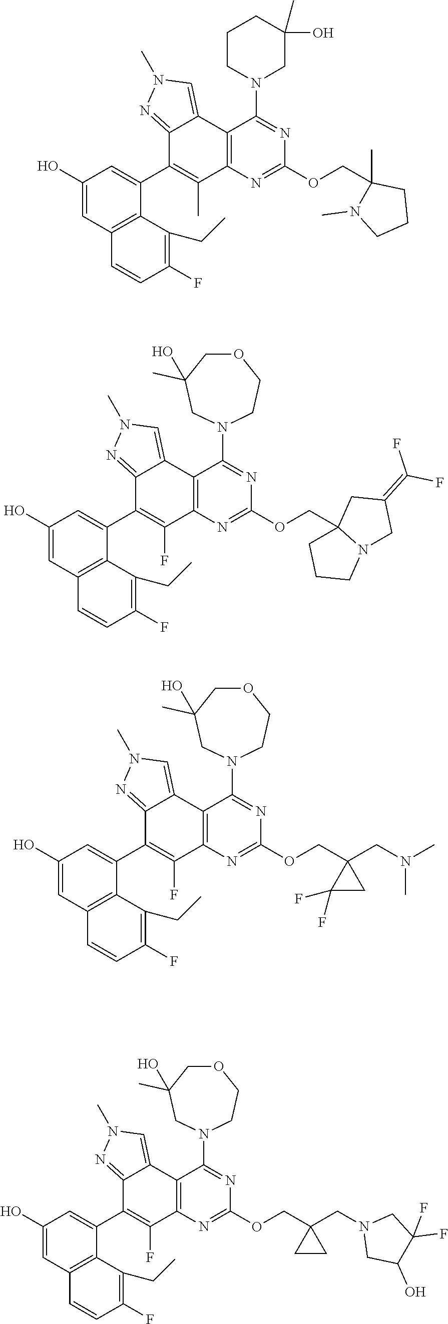 [SMILES: CCc1c(F)ccc2cc(O)cc(-c3c(C)c4nc(OCC5(C)CCCN5C)nc(N5CCCC(C)(O)C5)c4c4cn(C)nc34)c12 ; CCc1c(F)ccc2cc(O)cc(-c3c(F)c4nc(OCC56CCCN5CC(=C(F)F)C6)nc(N5CCOCC(C)(O)C5)c4c4cn(C)nc34)c12 ; CCc1c(F)ccc2cc(O)cc(-c3c(F)c4nc(OCC5(CN(C)C)CC5(F)F)nc(N5CCOCC(C)(O)C5)c4c4cn(C)nc34)c12 ; CCc1c(F)ccc2cc(O)cc(-c3c(F)c4nc(OCC5(CN6CC(O)C(F)(F)C6)CC5)nc(N5CCOCC(C)(O)C5)c4c4cn(C)nc34)c12]

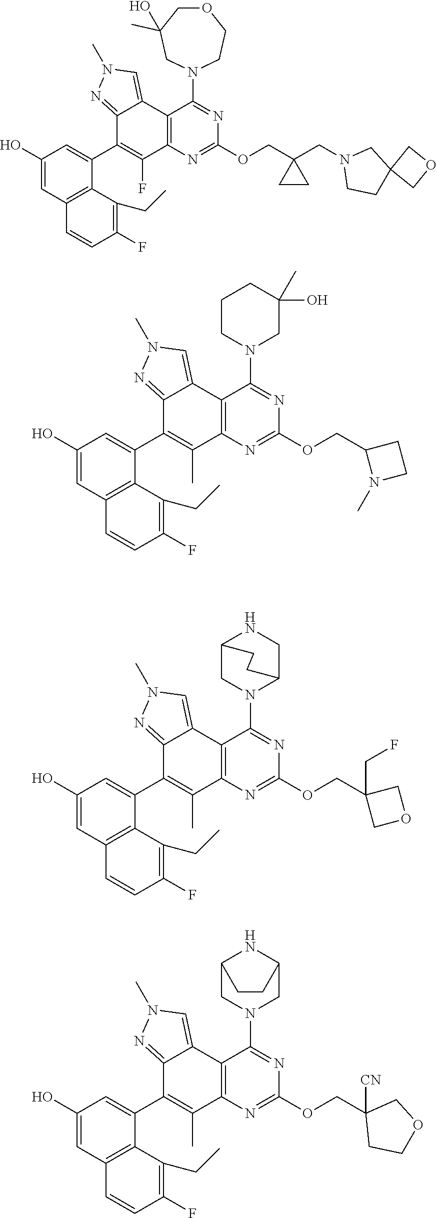 [SMILES: CCc1c(F)ccc2cc(O)cc(-c3c(F)c4nc(OCC5(CN6CCC7(COC7)C6)CC5)nc(N5CCOCC(C)(O)C5)c4c4cn(C)nc34)c12 ; CCc1c(F)ccc2cc(O)cc(-c3c(C)c4nc(OCC5CCN5C)nc(N5CCCC(C)(O)C5)c4c4cn(C)nc34)c12 ; CCc1c(F)ccc2cc(O)cc(-c3c(C)c4nc(OCC5(CF)COC5)nc(N5CC6CCC5CN6)c4c4cn(C)nc34)c12 ; CCc1c(F)ccc2cc(O)cc(-c3c(C)c4nc(OCC5(C#N)CCOC5)nc(N5CC6CCC(C5)N6)c4c4cn(C)nc34)c12]

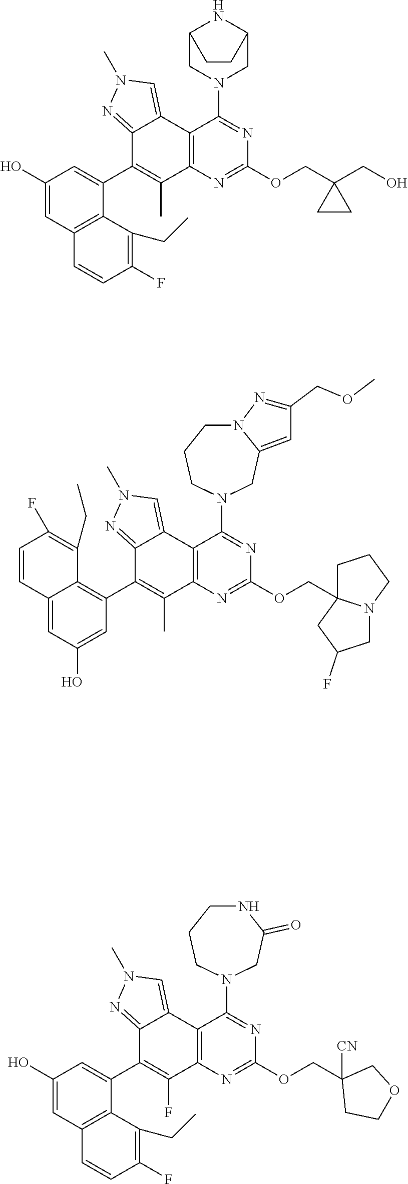 [SMILES: CCc1c(F)ccc2cc(O)cc(-c3c(C)c4nc(OCC5(CO)CC5)nc(N5CC6CCC(C5)N6)c4c4cn(C)nc34)c12 ; CCc1c(F)ccc2cc(O)cc(-c3c(C)c4nc(OCC56CCCN5CC(F)C6)nc(N5CCCn6nc(COC)cc6C5)c4c4cn(C)nc34)c12 ; CCc1c(F)ccc2cc(O)cc(-c3c(F)c4nc(OCC5(C#N)CCOC5)nc(N5CCCNC(=O)C5)c4c4cn(C)nc34)c12]

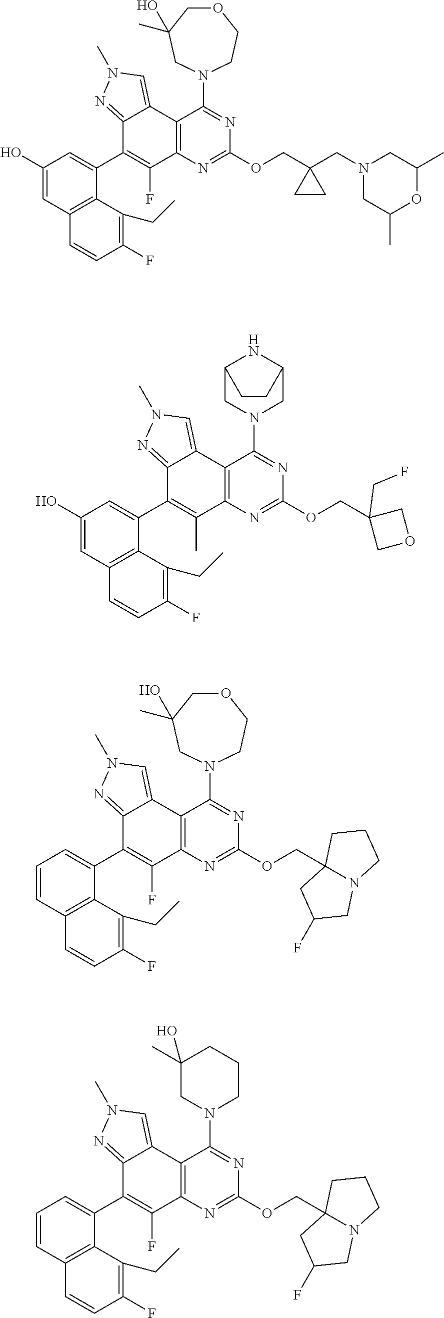 [SMILES: CCc1c(F)ccc2cc(O)cc(-c3c(F)c4nc(OCC5(CN6CC(C)OC(C)C6)CC5)nc(N5CCOCC(C)(O)C5)c4c4cn(C)nc34)c12 ; CCc1c(F)ccc2cc(O)cc(-c3c(C)c4nc(OCC5(CF)COC5)nc(N5CC6CCC(C5)N6)c4c4cn(C)nc34)c12 ; CCc1c(F)ccc2cccc(-c3c(F)c4nc(OCC56CCCN5CC(F)C6)nc(N5CCOCC(C)(O)C5)c4c4cn(C)nc34)c12 ; CCc1c(F)ccc2cccc(-c3c(F)c4nc(OCC56CCCN5CC(F)C6)nc(N5CCCC(C)(O)C5)c4c4cn(C)nc34)c12]

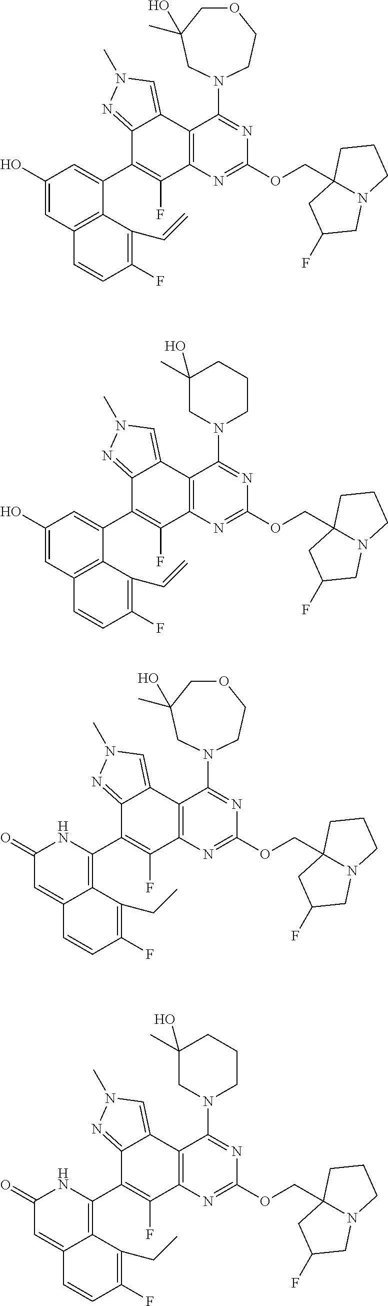 [SMILES: C=Cc1c(F)ccc2cc(O)cc(-c3c(F)c4nc(OCC56CCCN5CC(F)C6)nc(N5CCOCC(C)(O)C5)c4c4cn(C)nc34)c12 ; C=Cc1c(F)ccc2cc(O)cc(-c3c(F)c4nc(OCC56CCCN5CC(F)C6)nc(N5CCCC(C)(O)C5)c4c4cn(C)nc34)c12 ; CCc1c(F)ccc2cc(=O)[nH]c(-c3c(F)c4nc(OCC56CCCN5CC(F)C6)nc(N5CCOCC(C)(O)C5)c4c4cn(C)nc34)c12 ; CCc1c(F)ccc2cc(=O)[nH]c(-c3c(F)c4nc(OCC56CCCN5CC(F)C6)nc(N5CCCC(C)(O)C5)c4c4cn(C)nc34)c12]

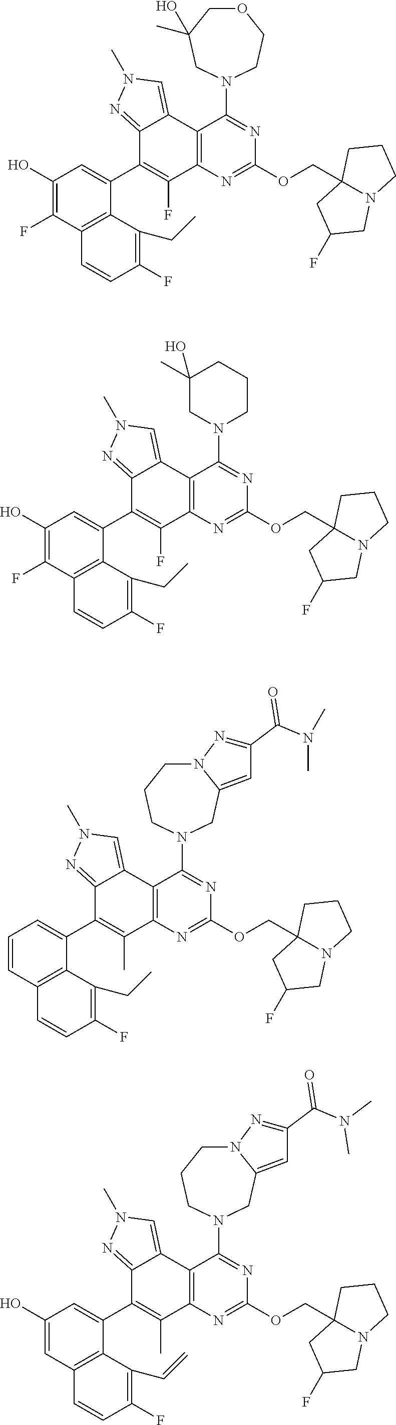 [SMILES: CCc1c(F)ccc2c(F)c(O)cc(-c3c(F)c4nc(OCC56CCCN5CC(F)C6)nc(N5CCOCC(C)(O)C5)c4c4cn(C)nc34)c12 ; CCc1c(F)ccc2c(F)c(O)cc(-c3c(F)c4nc(OCC56CCCN5CC(F)C6)nc(N5CCCC(C)(O)C5)c4c4cn(C)nc34)c12 ; CCc1c(F)ccc2cccc(-c3c(C)c4nc(OCC56CCCN5CC(F)C6)nc(N5CCCn6nc(C(=O)N(C)C)cc6C5)c4c4cn(C)nc34)c12 ; C=Cc1c(F)ccc2cc(O)cc(-c3c(C)c4nc(OCC56CCCN5CC(F)C6)nc(N5CCCn6nc(C(=O)N(C)C)cc6C5)c4c4cn(C)nc34)c12]

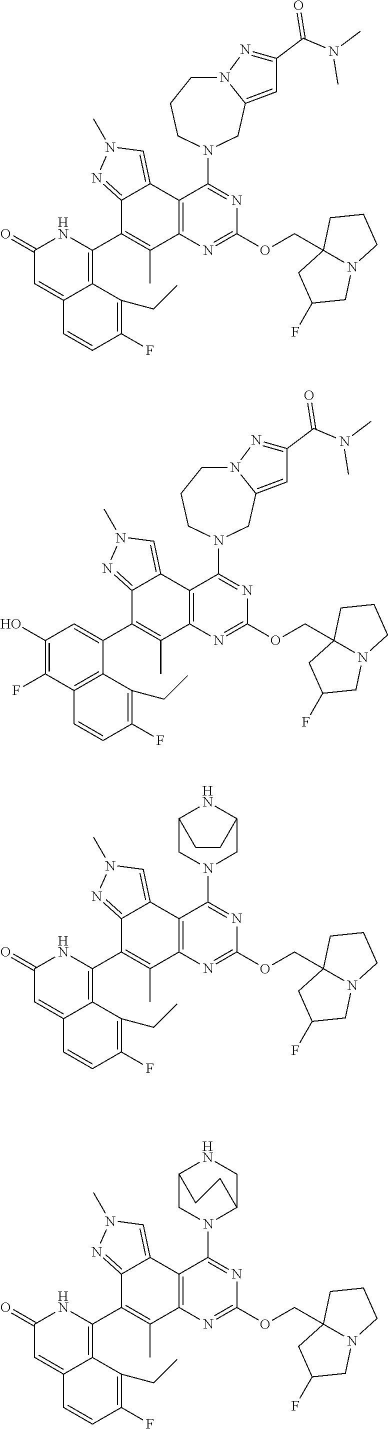 [SMILES: CCc1c(F)ccc2cc(=O)[nH]c(-c3c(C)c4nc(OCC56CCCN5CC(F)C6)nc(N5CCCn6nc(C(=O)N(C)C)cc6C5)c4c4cn(C)nc34)c12 ; CCc1c(F)ccc2c(F)c(O)cc(-c3c(C)c4nc(OCC56CCCN5CC(F)C6)nc(N5CCCn6nc(C(=O)N(C)C)cc6C5)c4c4cn(C)nc34)c12 ; CCc1c(F)ccc2cc(=O)[nH]c(-c3c(C)c4nc(OCC56CCCN5CC(F)C6)nc(N5CC6CCC(C5)N6)c4c4cn(C)nc34)c12 ; CCc1c(F)ccc2cc(=O)[nH]c(-c3c(C)c4nc(OCC56CCCN5CC(F)C6)nc(N5CC6CCC5CN6)c4c4cn(C)nc34)c12]

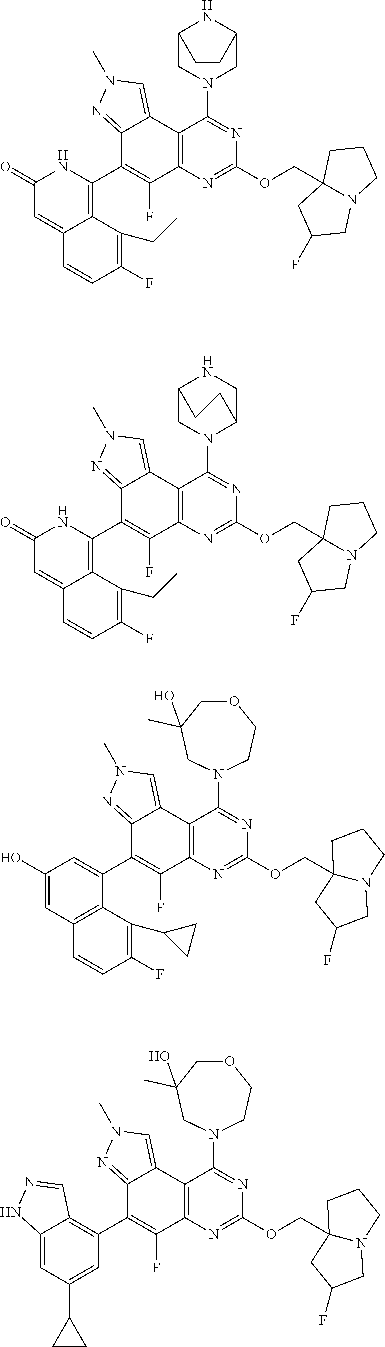 [SMILES: CCc1c(F)ccc2cc(=O)[nH]c(-c3c(F)c4nc(OCC56CCCN5CC(F)C6)nc(N5CC6CCC(C5)N6)c4c4cn(C)nc34)c12 ; CCc1c(F)ccc2cc(=O)[nH]c(-c3c(F)c4nc(OCC56CCCN5CC(F)C6)nc(N5CC6CCC5CN6)c4c4cn(C)nc34)c12 ; Cn1cc2c(n1)c(-c1cc(O)cc3ccc(F)c(C4CC4)c13)c(F)c1nc(OCC34CCCN3CC(F)C4)nc(N3CCOCC(C)(O)C3)c12 ; Cn1cc2c(n1)c(-c1cc(C3CC3)cc3[nH]ncc13)c(F)c1nc(OCC34CCCN3CC(F)C4)nc(N3CCOCC(C)(O)C3)c12]

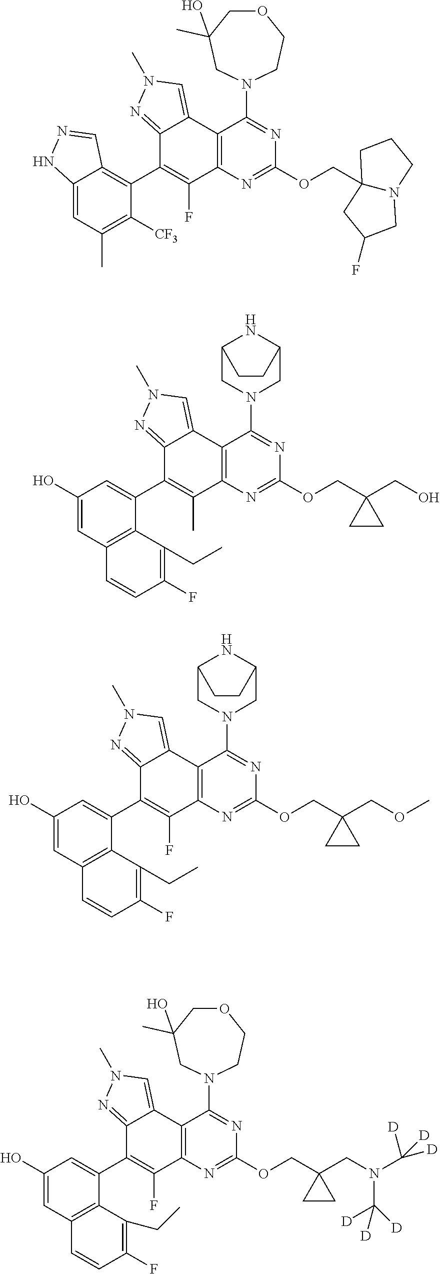 [SMILES: Cc1cc2[nH]ncc2c(-c2c(F)c3nc(OCC45CCCN4CC(F)C5)nc(N4CCOCC(C)(O)C4)c3c3cn(C)nc23)c1C(F)(F)F ; CCc1c(F)ccc2cc(O)cc(-c3c(C)c4nc(OCC5(CO)CC5)nc(N5CC6CCC(C5)N6)c4c4cn(C)nc34)c12 ; CCc1c(F)ccc2cc(O)cc(-c3c(F)c4nc(OCC5(COC)CC5)nc(N5CC6CCC(C5)N6)c4c4cn(C)nc34)c12 ; [2H]C([2H])([2H])N(CC1(COc2nc(N3CCOCC(C)(O)C3)c3c(n2)c(F)c(-c2cc(O)cc4ccc(F)c(CC)c24)c2nn(C)cc23)CC1)C([2H])([2H])[2H]]

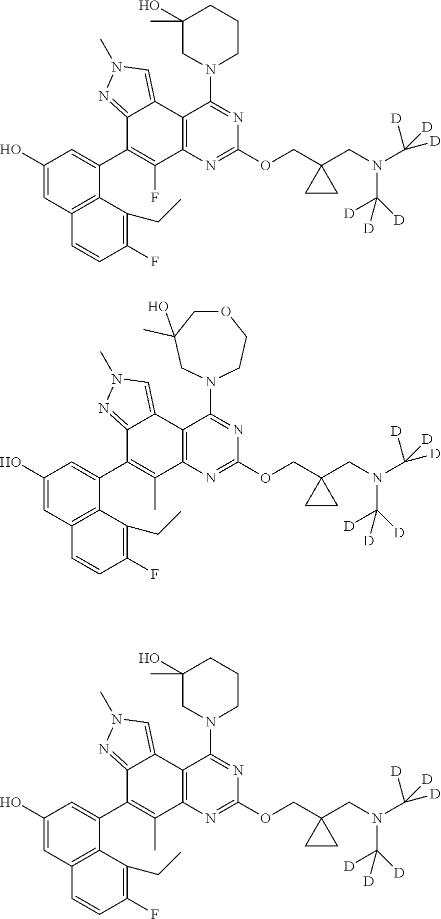 [SMILES: [2H]C([2H])([2H])N(CC1(COc2nc(N3CCCC(C)(O)C3)c3c(n2)c(F)c(-c2cc(O)cc4ccc(F)c(CC)c24)c2nn(C)cc23)CC1)C([2H])([2H])[2H] ; [2H]C([2H])([2H])N(CC1(COc2nc(N3CCOCC(C)(O)C3)c3c(n2)c(C)c(-c2cc(O)cc4ccc(F)c(CC)c24)c2nn(C)cc23)CC1)C([2H])([2H])[2H] ; [2H]C([2H])([2H])N(CC1(COc2nc(N3CCCC(C)(O)C3)c3c(n2)c(C)c(-c2cc(O)cc4ccc(F)c(CC)c24)c2nn(C)cc23)CC1)C([2H])([2H])[2H]]

The compound of formula (I) may be selected from:

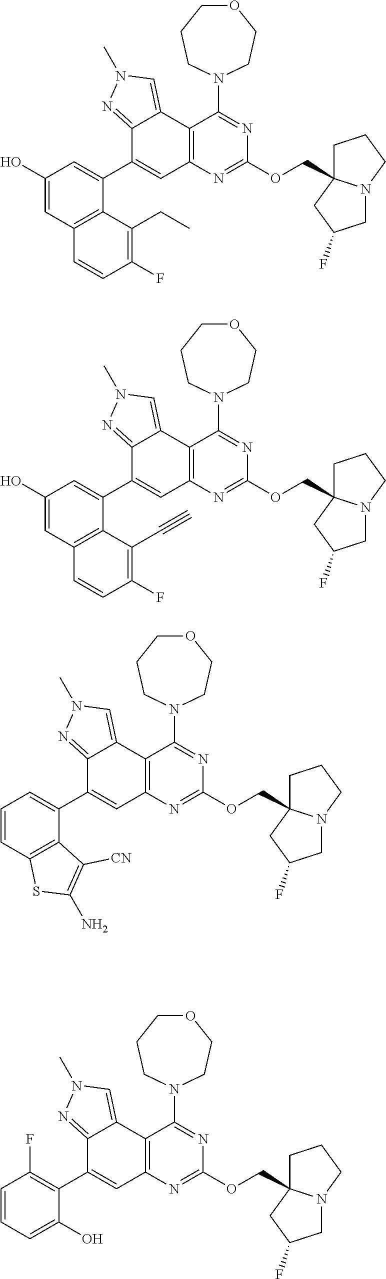

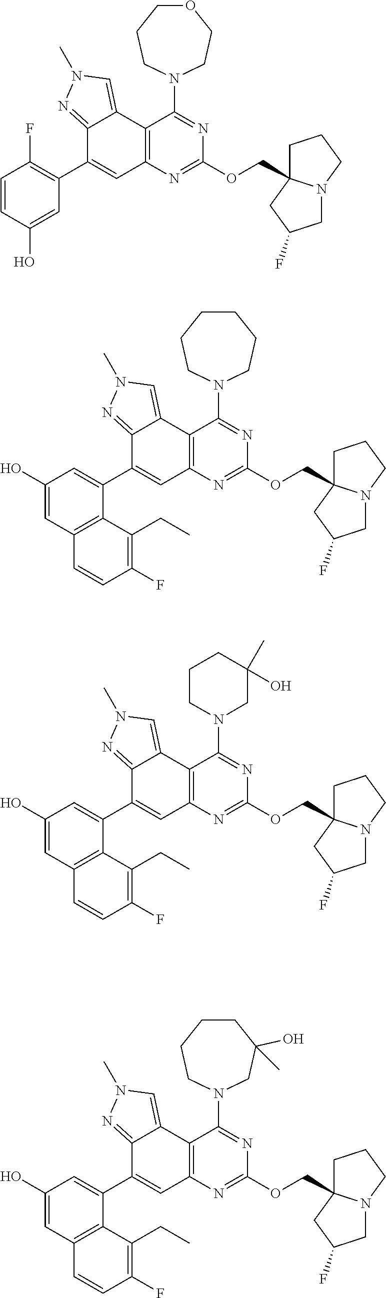 [SMILES: Cn1cc2c(n1)c(-c1cc(O)ccc1F)cc1nc(OC[C@@]34CCCN3C[C@H](F)C4)nc(N3CCCOCC3)c12 ; CCc1c(F)ccc2cc(O)cc(-c3cc4nc(OC[C@@]56CCCN5C[C@H](F)C6)nc(N5CCCCCC5)c4c4cn(C)nc34)c12 ; CCc1c(F)ccc2cc(O)cc(-c3cc4nc(OC[C@@]56CCCN5C[C@H](F)C6)nc(N5CCCC(C)(O)C5)c4c4cn(C)nc34)c12 ; CCc1c(F)ccc2cc(O)cc(-c3cc4nc(OC[C@@]56CCCN5C[C@H](F)C6)nc(N5CCCCC(C)(O)C5)c4c4cn(C)nc34)c12]

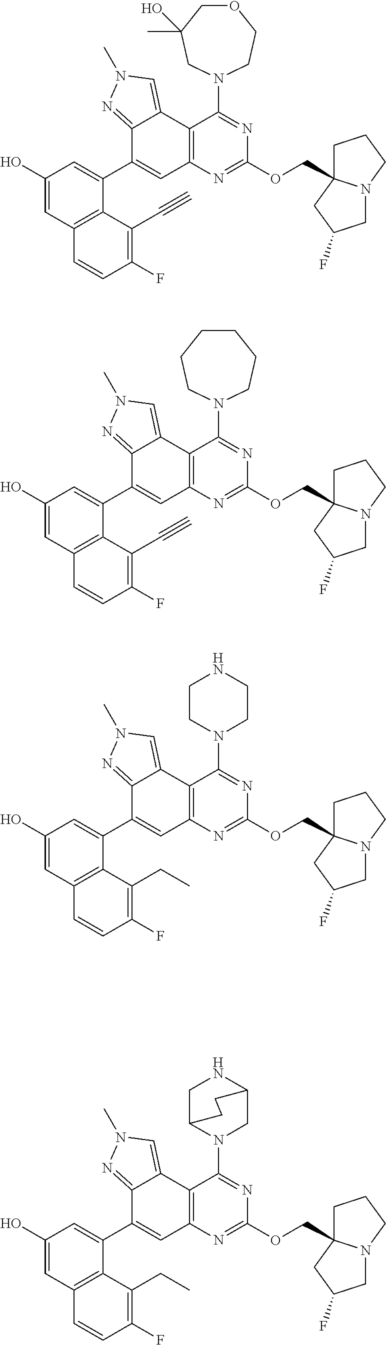 [SMILES: C#Cc1c(F)ccc2cc(O)cc(-c3cc4nc(OC[C@@]56CCCN5C[C@H](F)C6)nc(N5CCOCC(C)(O)C5)c4c4cn(C)nc34)c12 ; C#Cc1c(F)ccc2cc(O)cc(-c3cc4nc(OC[C@@]56CCCN5C[C@H](F)C6)nc(N5CCCCCC5)c4c4cn(C)nc34)c12 ; CCc1c(F)ccc2cc(O)cc(-c3cc4nc(OC[C@@]56CCCN5C[C@H](F)C6)nc(N5CCNCC5)c4c4cn(C)nc34)c12 ; CCc1c(F)ccc2cc(O)cc(-c3cc4nc(OC[C@@]56CCCN5C[C@H](F)C6)nc(N5CC6CCC5CN6)c4c4cn(C)nc34)c12]

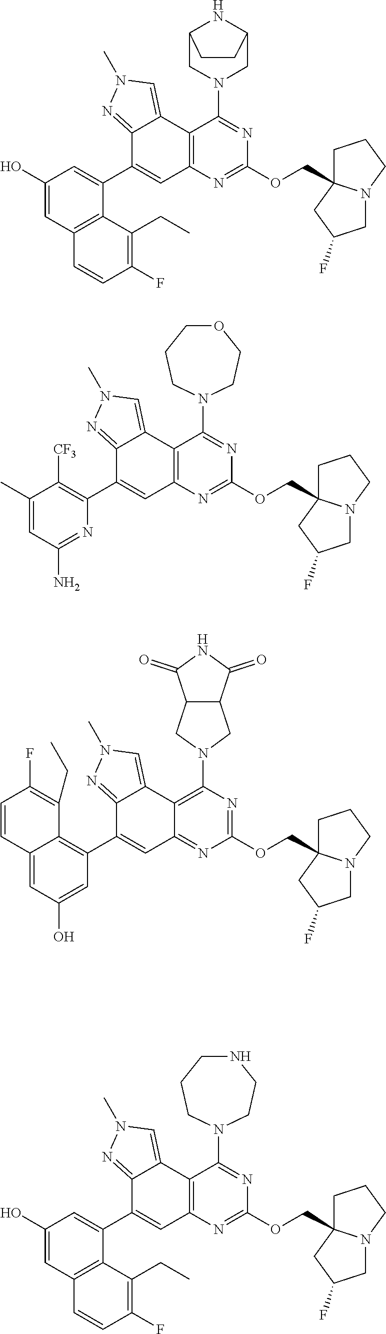 [SMILES: CCc1c(F)ccc2cc(O)cc(-c3cc4nc(OC[C@@]56CCCN5C[C@H](F)C6)nc(N5CC6CCC(C5)N6)c4c4cn(C)nc34)c12 ; Cc1cc(N)nc(-c2cc3nc(OC[C@@]45CCCN4C[C@H](F)C5)nc(N4CCCOCC4)c3c3cn(C)nc23)c1C(F)(F)F ; CCc1c(F)ccc2cc(O)cc(-c3cc4nc(OC[C@@]56CCCN5C[C@H](F)C6)nc(N5CC6C(=O)NC(=O)C6C5)c4c4cn(C)nc34)c12 ; CCc1c(F)ccc2cc(O)cc(-c3cc4nc(OC[C@@]56CCCN5C[C@H](F)C6)nc(N5CCCNCC5)c4c4cn(C)nc34)c12]

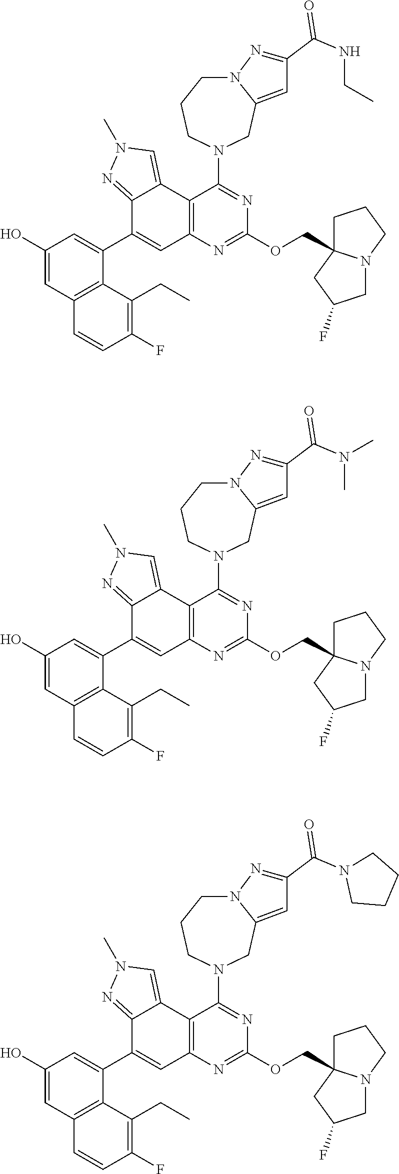 [SMILES: CCNC(=O)c1cc2n(n1)CCCN(c1nc(OC[C@@]34CCCN3C[C@H](F)C4)nc3cc(-c4cc(O)cc5ccc(F)c(CC)c45)c4nn(C)cc4c13)C2 ; CCc1c(F)ccc2cc(O)cc(-c3cc4nc(OC[C@@]56CCCN5C[C@H](F)C6)nc(N5CCCn6nc(C(=O)N(C)C)cc6C5)c4c4cn(C)nc34)c12 ; CCc1c(F)ccc2cc(O)cc(-c3cc4nc(OC[C@@]56CCCN5C[C@H](F)C6)nc(N5CCCn6nc(C(=O)N7CCCC7)cc6C5)c4c4cn(C)nc34)c12]

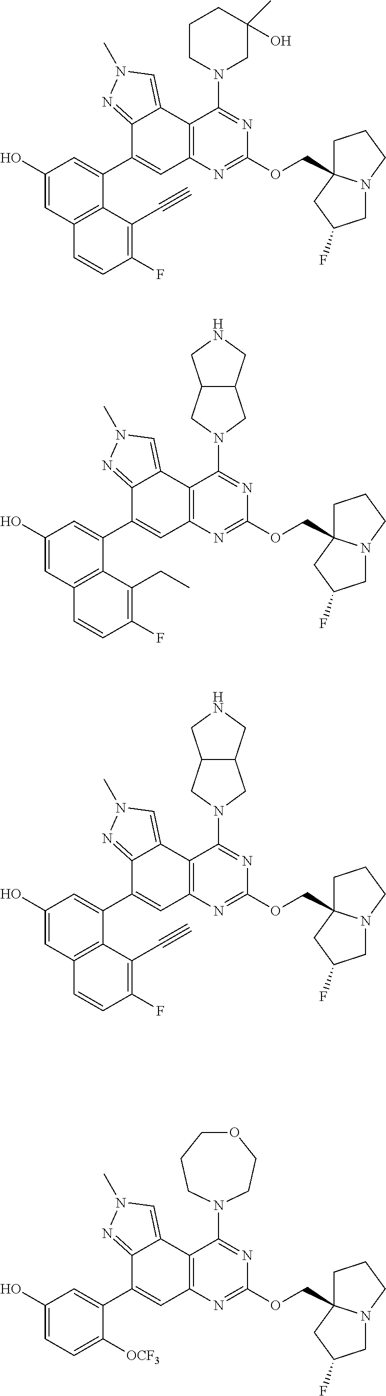 [SMILES: C#Cc1c(F)ccc2cc(O)cc(-c3cc4nc(OC[C@@]56CCCN5C[C@H](F)C6)nc(N5CCCC(C)(O)C5)c4c4cn(C)nc34)c12 ; CCc1c(F)ccc2cc(O)cc(-c3cc4nc(OC[C@@]56CCCN5C[C@H](F)C6)nc(N5CC6CNCC6C5)c4c4cn(C)nc34)c12 ; C#Cc1c(F)ccc2cc(O)cc(-c3cc4nc(OC[C@@]56CCCN5C[C@H](F)C6)nc(N5CC6CNCC6C5)c4c4cn(C)nc34)c12 ; Cn1cc2c(n1)c(-c1cc(O)ccc1OC(F)(F)F)cc1nc(OC[C@@]34CCCN3C[C@H](F)C4)nc(N3CCCOCC3)c12]

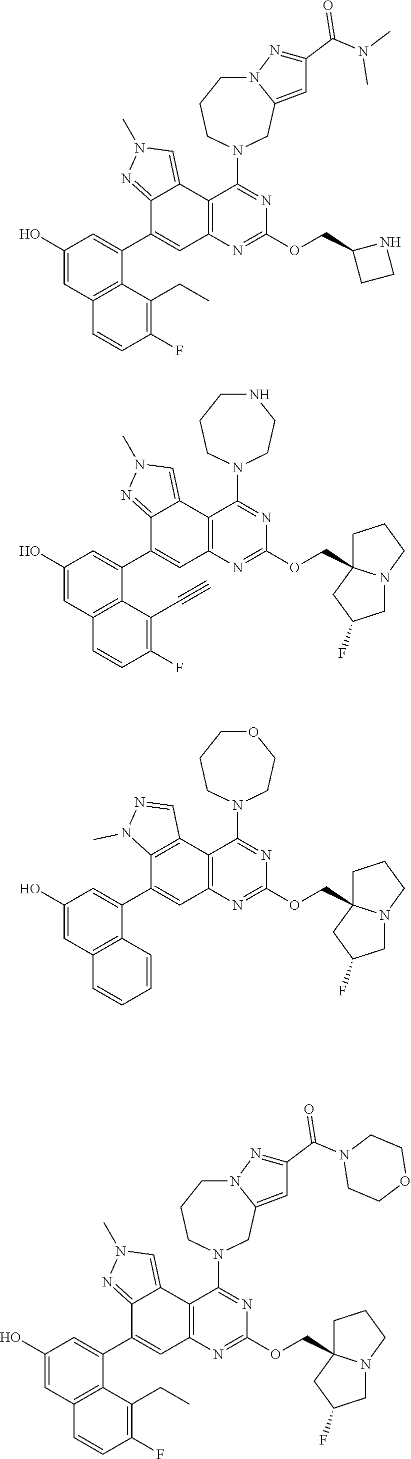 [SMILES: CCc1c(F)ccc2cc(O)cc(-c3cc4nc(OC[C@@H]5CCN5)nc(N5CCCn6nc(C(=O)N(C)C)cc6C5)c4c4cn(C)nc34)c12 ; C#Cc1c(F)ccc2cc(O)cc(-c3cc4nc(OC[C@@]56CCCN5C[C@H](F)C6)nc(N5CCCNCC5)c4c4cn(C)nc34)c12 ; Cn1ncc2c3c(N4CCCOCC4)nc(OC[C@@]45CCCN4C[C@H](F)C5)nc3cc(-c3cc(O)cc4ccccc34)c21 ; CCc1c(F)ccc2cc(O)cc(-c3cc4nc(OC[C@@]56CCCN5C[C@H](F)C6)nc(N5CCCn6nc(C(=O)N7CCOCC7)cc6C5)c4c4cn(C)nc34)c12]

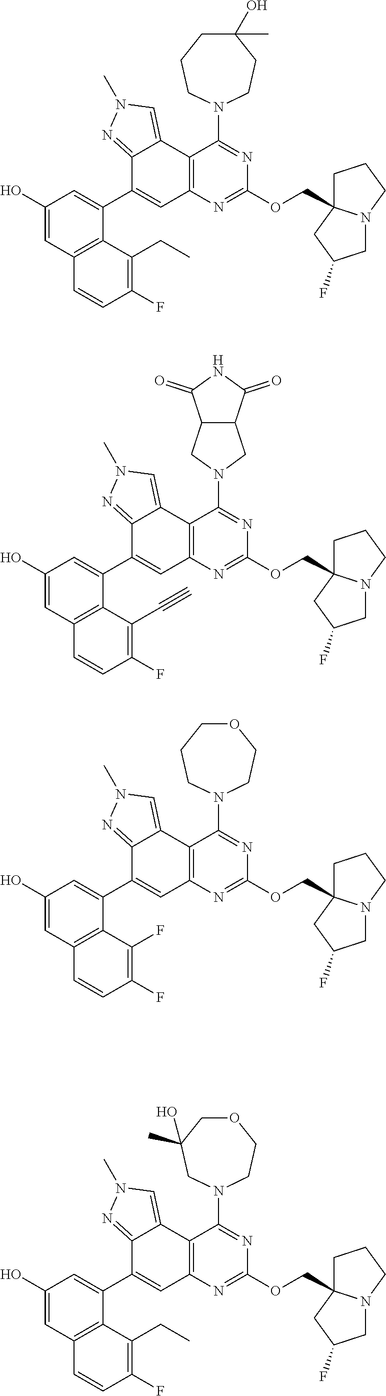 [SMILES: CCc1c(F)ccc2cc(O)cc(-c3cc4nc(OC[C@@]56CCCN5C[C@H](F)C6)nc(N5CCCC(C)(O)CC5)c4c4cn(C)nc34)c12 ; C#Cc1c(F)ccc2cc(O)cc(-c3cc4nc(OC[C@@]56CCCN5C[C@H](F)C6)nc(N5CC6C(=O)NC(=O)C6C5)c4c4cn(C)nc34)c12 ; Cn1cc2c(n1)c(-c1cc(O)cc3ccc(F)c(F)c13)cc1nc(OC[C@@]34CCCN3C[C@H](F)C4)nc(N3CCCOCC3)c12 ; CCc1c(F)ccc2cc(O)cc(-c3cc4nc(OC[C@@]56CCCN5C[C@H](F)C6)nc(N5CCOC[C@@](C)(O)C5)c4c4cn(C)nc34)c12]

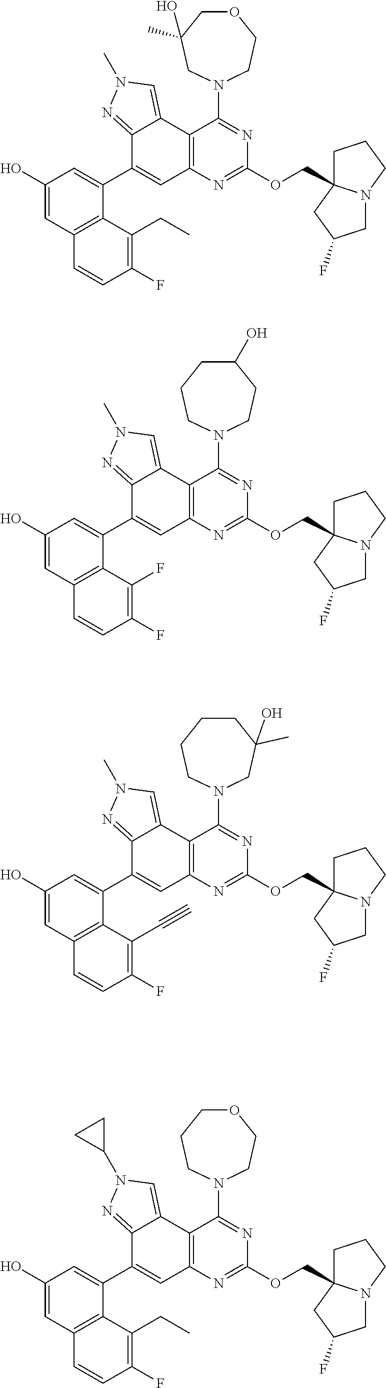 [SMILES: CCc1c(F)ccc2cc(O)cc(-c3cc4nc(OC[C@@]56CCCN5C[C@H](F)C6)nc(N5CCOC[C@](C)(O)C5)c4c4cn(C)nc34)c12 ; Cn1cc2c(n1)c(-c1cc(O)cc3ccc(F)c(F)c13)cc1nc(OC[C@@]34CCCN3C[C@H](F)C4)nc(N3CCCC(O)CC3)c12 ; C#Cc1c(F)ccc2cc(O)cc(-c3cc4nc(OC[C@@]56CCCN5C[C@H](F)C6)nc(N5CCCCC(C)(O)C5)c4c4cn(C)nc34)c12 ; CCc1c(F)ccc2cc(O)cc(-c3cc4nc(OC[C@@]56CCCN5C[C@H](F)C6)nc(N5CCCOCC5)c4c4cn(C5CC5)nc34)c12]

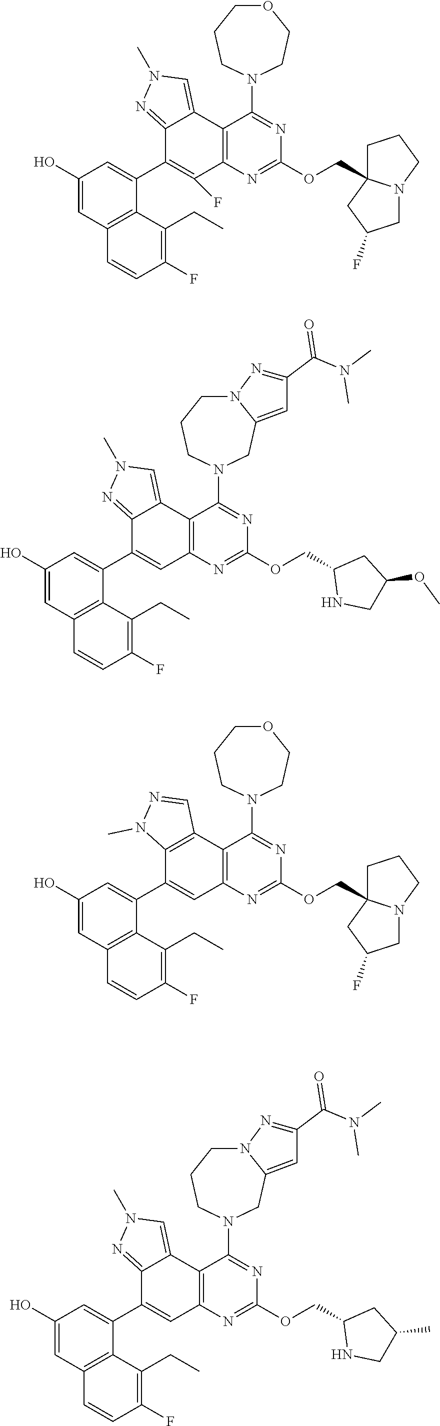 [SMILES: CCc1c(F)ccc2cc(O)cc(-c3c(F)c4nc(OC[C@@]56CCCN5C[C@H](F)C6)nc(N5CCCOCC5)c4c4cn(C)nc34)c12 ; CCc1c(F)ccc2cc(O)cc(-c3cc4nc(OC[C@@H]5C[C@@H](OC)CN5)nc(N5CCCn6nc(C(=O)N(C)C)cc6C5)c4c4cn(C)nc34)c12 ; CCc1c(F)ccc2cc(O)cc(-c3cc4nc(OC[C@@]56CCCN5C[C@H](F)C6)nc(N5CCCOCC5)c4c4cnn(C)c34)c12 ; CCc1c(F)ccc2cc(O)cc(-c3cc4nc(OC[C@@H]5C[C@H](C)CN5)nc(N5CCCn6nc(C(=O)N(C)C)cc6C5)c4c4cn(C)nc34)c12]

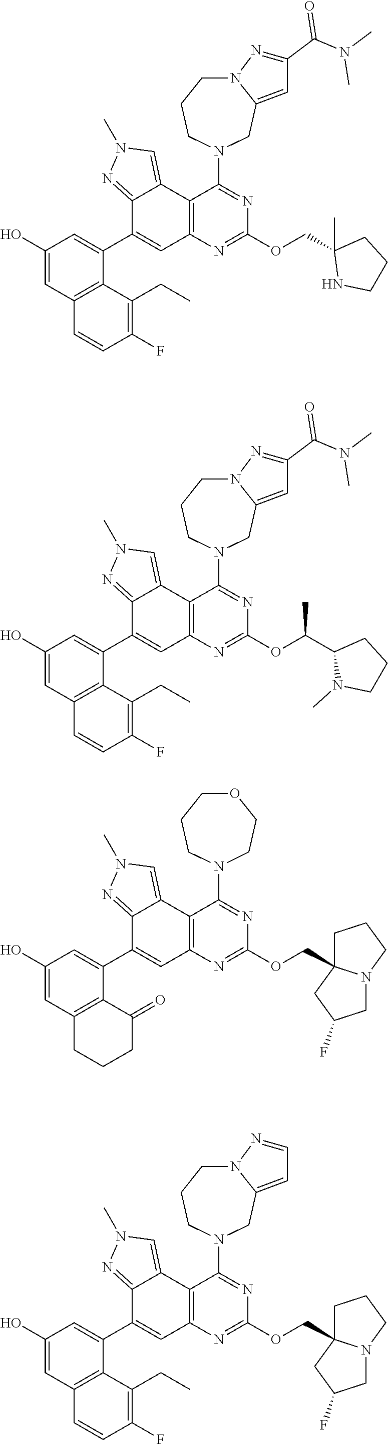 [SMILES: CCc1c(F)ccc2cc(O)cc(-c3cc4nc(OC[C@]5(C)CCCN5)nc(N5CCCn6nc(C(=O)N(C)C)cc6C5)c4c4cn(C)nc34)c12 ; CCc1c(F)ccc2cc(O)cc(-c3cc4nc(O[C@@H](C)[C@@H]5CCCN5C)nc(N5CCCn6nc(C(=O)N(C)C)cc6C5)c4c4cn(C)nc34)c12 ; Cn1cc2c(n1)c(-c1cc(O)cc3c1C(=O)CCC3)cc1nc(OC[C@@]34CCCN3C[C@H](F)C4)nc(N3CCCOCC3)c12 ; CCc1c(F)ccc2cc(O)cc(-c3cc4nc(OC[C@@]56CCCN5C[C@H](F)C6)nc(N5CCCn6nccc6C5)c4c4cn(C)nc34)c12]

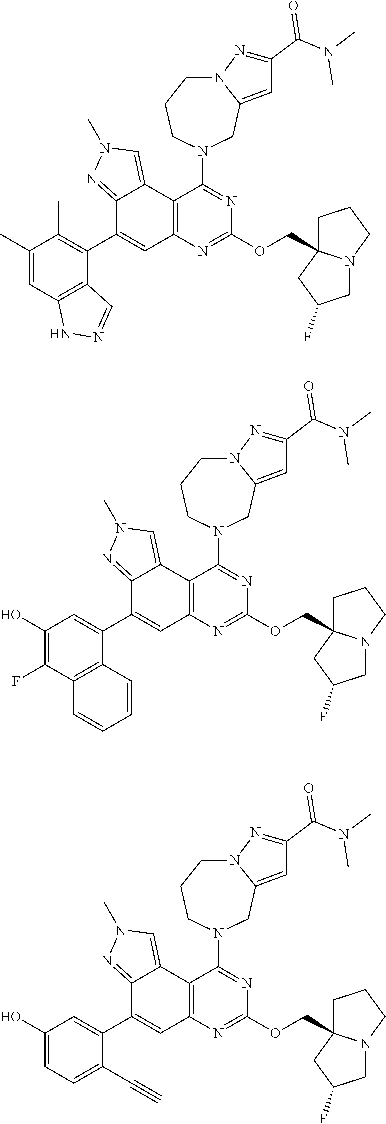 [SMILES: Cc1cc2[nH]ncc2c(-c2cc3nc(OC[C@@]45CCCN4C[C@H](F)C5)nc(N4CCCn5nc(C(=O)N(C)C)cc5C4)c3c3cn(C)nc23)c1C ; CN(C)C(=O)c1cc2n(n1)CCCN(c1nc(OC[C@@]34CCCN3C[C@H](F)C4)nc3cc(-c4cc(O)c(F)c5ccccc45)c4nn(C)cc4c13)C2 ; C#Cc1ccc(O)cc1-c1cc2nc(OC[C@@]34CCCN3C[C@H](F)C4)nc(N3CCCn4nc(C(=O)N(C)C)cc4C3)c2c2cn(C)nc12]

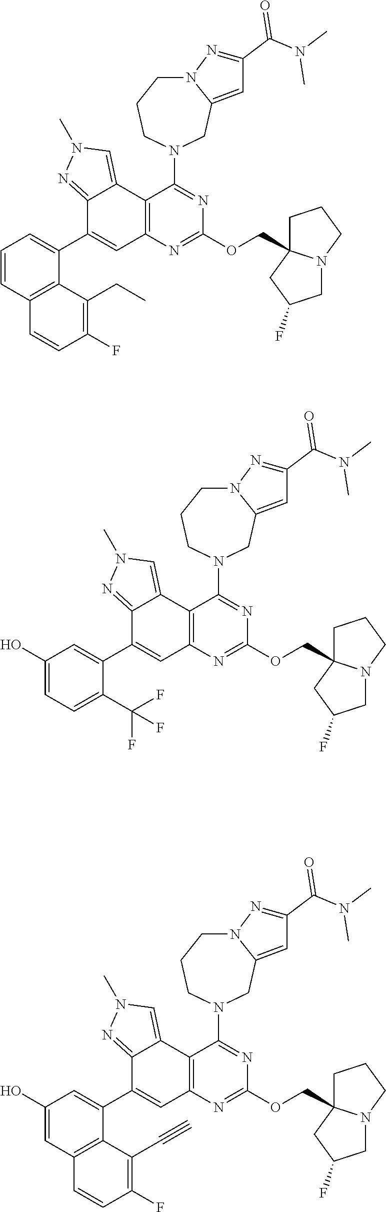 [SMILES: CCc1c(F)ccc2cccc(-c3cc4nc(OC[C@@]56CCCN5C[C@H](F)C6)nc(N5CCCn6nc(C(=O)N(C)C)cc6C5)c4c4cn(C)nc34)c12 ; CN(C)C(=O)c1cc2n(n1)CCCN(c1nc(OC[C@@]34CCCN3C[C@H](F)C4)nc3cc(-c4cc(O)ccc4C(F)(F)F)c4nn(C)cc4c13)C2 ; C#Cc1c(F)ccc2cc(O)cc(-c3cc4nc(OC[C@@]56CCCN5C[C@H](F)C6)nc(N5CCCn6nc(C(=O)N(C)C)cc6C5)c4c4cn(C)nc34)c12]

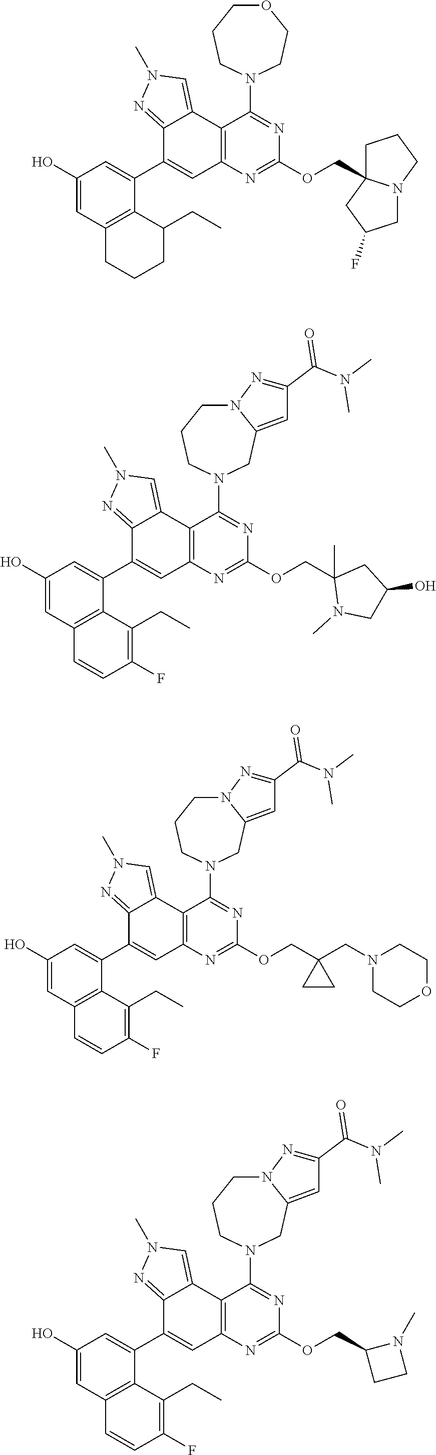 [SMILES: CCC1CCCc2cc(O)cc(-c3cc4nc(OC[C@@]56CCCN5C[C@H](F)C6)nc(N5CCCOCC5)c4c4cn(C)nc34)c21 ; CCc1c(F)ccc2cc(O)cc(-c3cc4nc(OCC5(C)C[C@@H](O)CN5C)nc(N5CCCn6nc(C(=O)N(C)C)cc6C5)c4c4cn(C)nc34)c12 ; CCc1c(F)ccc2cc(O)cc(-c3cc4nc(OCC5(CN6CCOCC6)CC5)nc(N5CCCn6nc(C(=O)N(C)C)cc6C5)c4c4cn(C)nc34)c12 ; CCc1c(F)ccc2cc(O)cc(-c3cc4nc(OC[C@@H]5CCN5C)nc(N5CCCn6nc(C(=O)N(C)C)cc6C5)c4c4cn(C)nc34)c12]

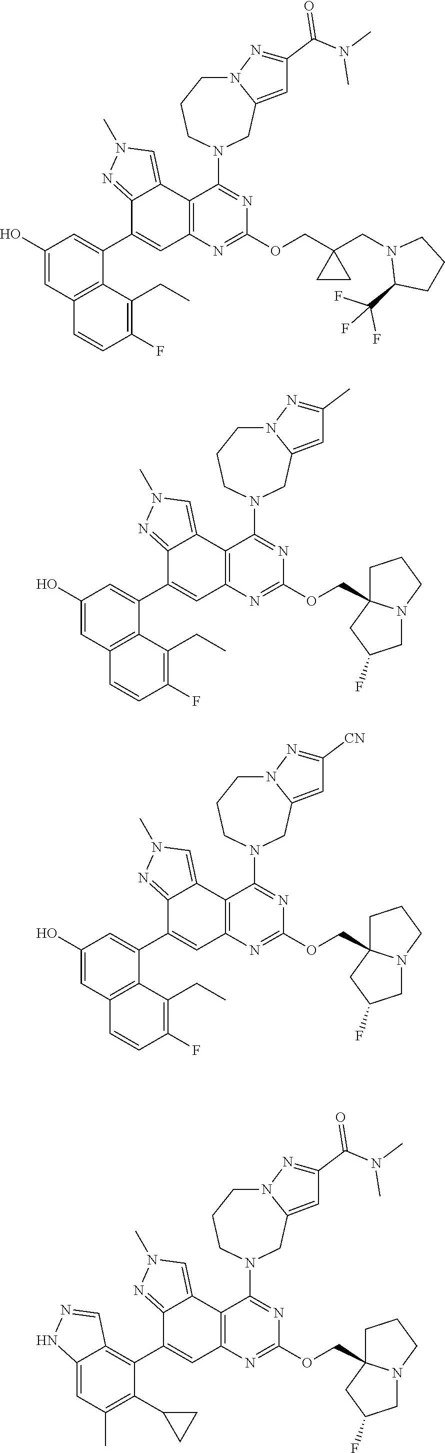 [SMILES: CCc1c(F)ccc2cc(O)cc(-c3cc4nc(OCC5(CN6CCC[C@H]6C(F)(F)F)CC5)nc(N5CCCn6nc(C(=O)N(C)C)cc6C5)c4c4cn(C)nc34)c12 ; CCc1c(F)ccc2cc(O)cc(-c3cc4nc(OC[C@@]56CCCN5C[C@H](F)C6)nc(N5CCCn6nc(C)cc6C5)c4c4cn(C)nc34)c12 ; CCc1c(F)ccc2cc(O)cc(-c3cc4nc(OC[C@@]56CCCN5C[C@H](F)C6)nc(N5CCCn6nc(C#N)cc6C5)c4c4cn(C)nc34)c12 ; Cc1cc2[nH]ncc2c(-c2cc3nc(OC[C@@]45CCCN4C[C@H](F)C5)nc(N4CCCn5nc(C(=O)N(C)C)cc5C4)c3c3cn(C)nc23)c1C1CC1]

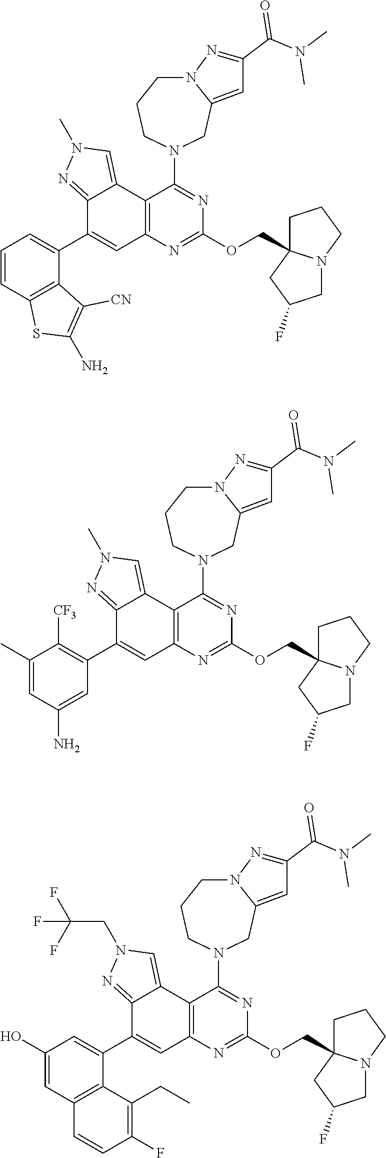 [SMILES: CN(C)C(=O)c1cc2n(n1)CCCN(c1nc(OC[C@@]34CCCN3C[C@H](F)C4)nc3cc(-c4cccc5sc(N)c(C#N)c45)c4nn(C)cc4c13)C2 ; Cc1cc(N)cc(-c2cc3nc(OC[C@@]45CCCN4C[C@H](F)C5)nc(N4CCCn5nc(C(=O)N(C)C)cc5C4)c3c3cn(C)nc23)c1C(F)(F)F ; CCc1c(F)ccc2cc(O)cc(-c3cc4nc(OC[C@@]56CCCN5C[C@H](F)C6)nc(N5CCCn6nc(C(=O)N(C)C)cc6C5)c4c4cn(CC(F)(F)F)nc34)c12]

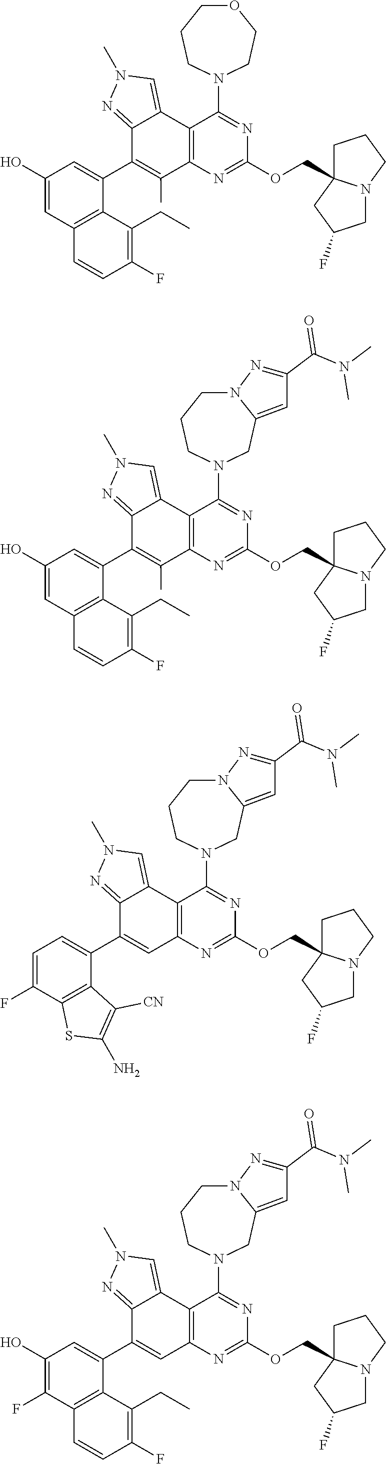 [SMILES: CCc1c(F)ccc2cc(O)cc(-c3c(C)c4nc(OC[C@@]56CCCN5C[C@H](F)C6)nc(N5CCCOCC5)c4c4cn(C)nc34)c12 ; CCc1c(F)ccc2cc(O)cc(-c3c(C)c4nc(OC[C@@]56CCCN5C[C@H](F)C6)nc(N5CCCn6nc(C(=O)N(C)C)cc6C5)c4c4cn(C)nc34)c12 ; CN(C)C(=O)c1cc2n(n1)CCCN(c1nc(OC[C@@]34CCCN3C[C@H](F)C4)nc3cc(-c4ccc(F)c5sc(N)c(C#N)c45)c4nn(C)cc4c13)C2 ; CCc1c(F)ccc2c(F)c(O)cc(-c3cc4nc(OC[C@@]56CCCN5C[C@H](F)C6)nc(N5CCCn6nc(C(=O)N(C)C)cc6C5)c4c4cn(C)nc34)c12]

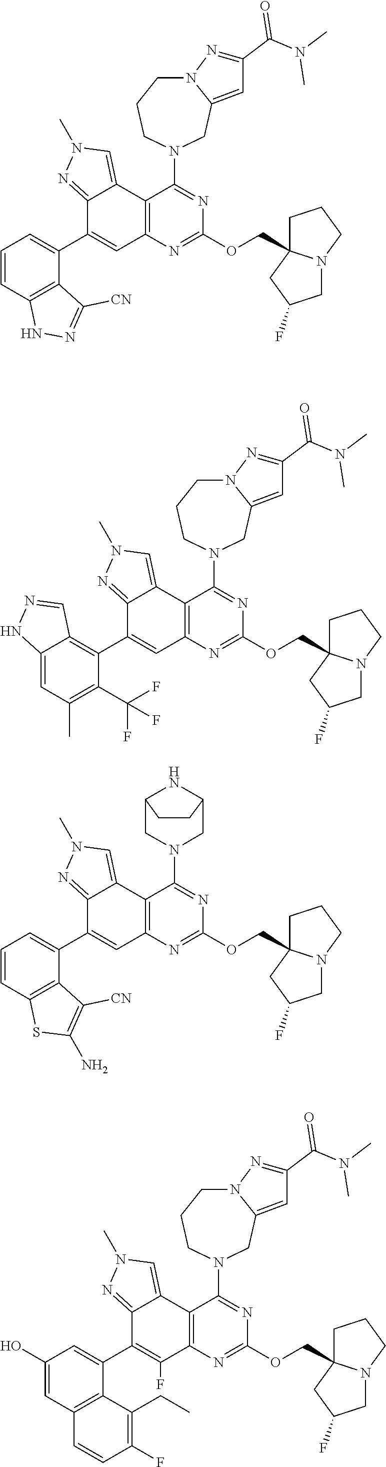 [SMILES: CN(C)C(=O)c1cc2n(n1)CCCN(c1nc(OC[C@@]34CCCN3C[C@H](F)C4)nc3cc(-c4cccc5[nH]nc(C#N)c45)c4nn(C)cc4c13)C2 ; Cc1cc2[nH]ncc2c(-c2cc3nc(OC[C@@]45CCCN4C[C@H](F)C5)nc(N4CCCn5nc(C(=O)N(C)C)cc5C4)c3c3cn(C)nc23)c1C(F)(F)F ; Cn1cc2c(n1)c(-c1cccc3sc(N)c(C#N)c13)cc1nc(OC[C@@]34CCCN3C[C@H](F)C4)nc(N3CC4CCC(C3)N4)c12 ; CCc1c(F)ccc2cc(O)cc(-c3c(F)c4nc(OC[C@@]56CCCN5C[C@H](F)C6)nc(N5CCCn6nc(C(=O)N(C)C)cc6C5)c4c4cn(C)nc34)c12]

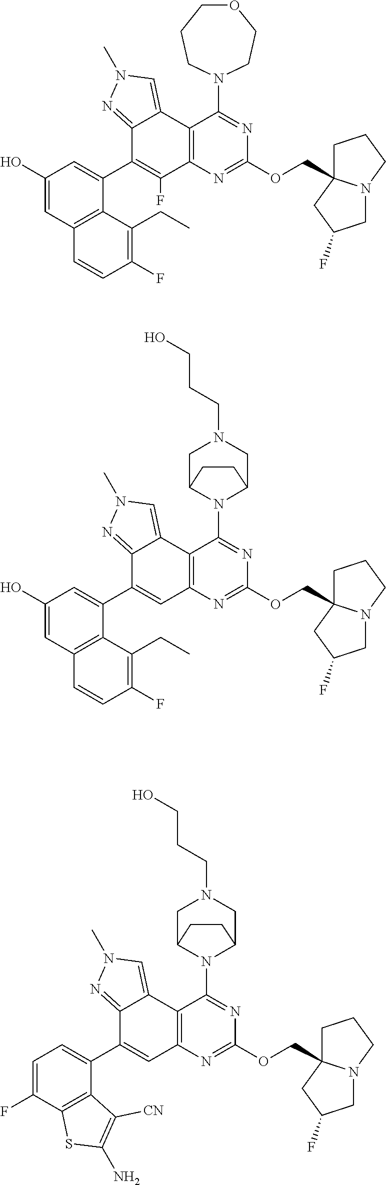 [SMILES: CCc1c(F)ccc2cc(O)cc(-c3c(F)c4nc(OC[C@@]56CCCN5C[C@H](F)C6)nc(N5CCCOCC5)c4c4cn(C)nc34)c12 ; CCc1c(F)ccc2cc(O)cc(-c3cc4nc(OC[C@@]56CCCN5C[C@H](F)C6)nc(N5C6CCC5CN(CCCO)C6)c4c4cn(C)nc34)c12 ; Cn1cc2c(n1)c(-c1ccc(F)c3sc(N)c(C#N)c13)cc1nc(OC[C@@]34CCCN3C[C@H](F)C4)nc(N3C4CCC3CN(CCCO)C4)c12]

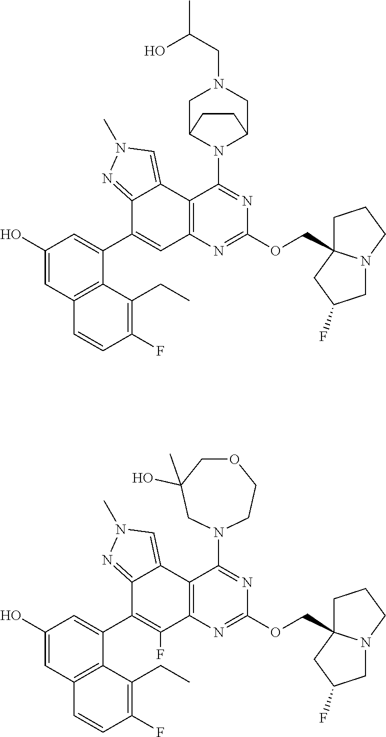 [SMILES: CCc1c(F)ccc2cc(O)cc(-c3cc4nc(OC[C@@]56CCCN5C[C@H](F)C6)nc(N5C6CCC5CN(CC(C)O)C6)c4c4cn(C)nc34)c12 ; CCc1c(F)ccc2cc(O)cc(-c3c(F)c4nc(OC[C@@]56CCCN5C[C@H](F)C6)nc(N5CCOCC(C)(O)C5)c4c4cn(C)nc34)c12]

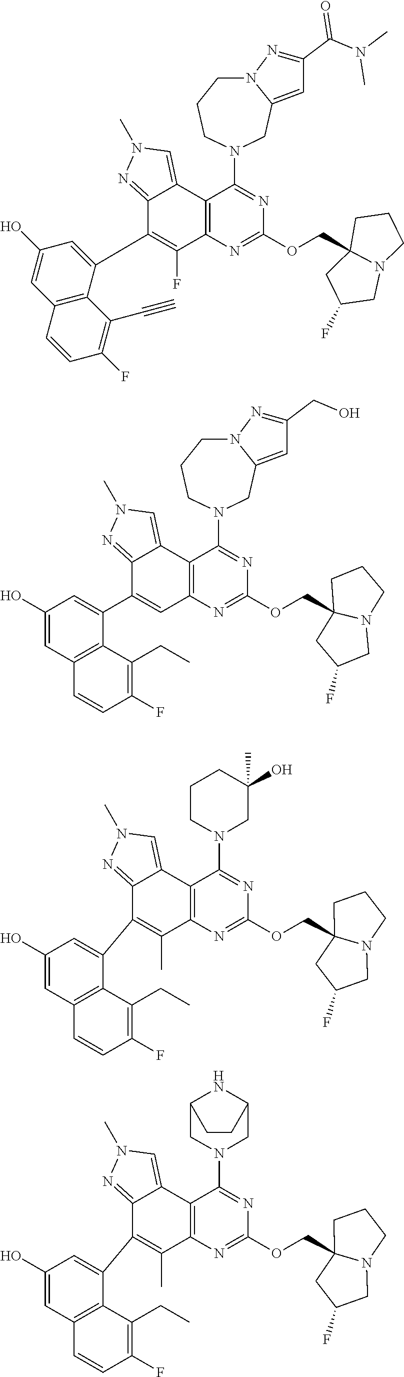 [SMILES: C#Cc1c(F)ccc2cc(O)cc(-c3c(F)c4nc(OC[C@@]56CCCN5C[C@H](F)C6)nc(N5CCCn6nc(C(=O)N(C)C)cc6C5)c4c4cn(C)nc34)c12 ; CCc1c(F)ccc2cc(O)cc(-c3cc4nc(OC[C@@]56CCCN5C[C@H](F)C6)nc(N5CCCn6nc(CO)cc6C5)c4c4cn(C)nc34)c12 ; CCc1c(F)ccc2cc(O)cc(-c3c(C)c4nc(OC[C@@]56CCCN5C[C@H](F)C6)nc(N5CCC[C@@](C)(O)C5)c4c4cn(C)nc34)c12 ; CCc1c(F)ccc2cc(O)cc(-c3c(C)c4nc(OC[C@@]56CCCN5C[C@H](F)C6)nc(N5CC6CCC(C5)N6)c4c4cn(C)nc34)c12]

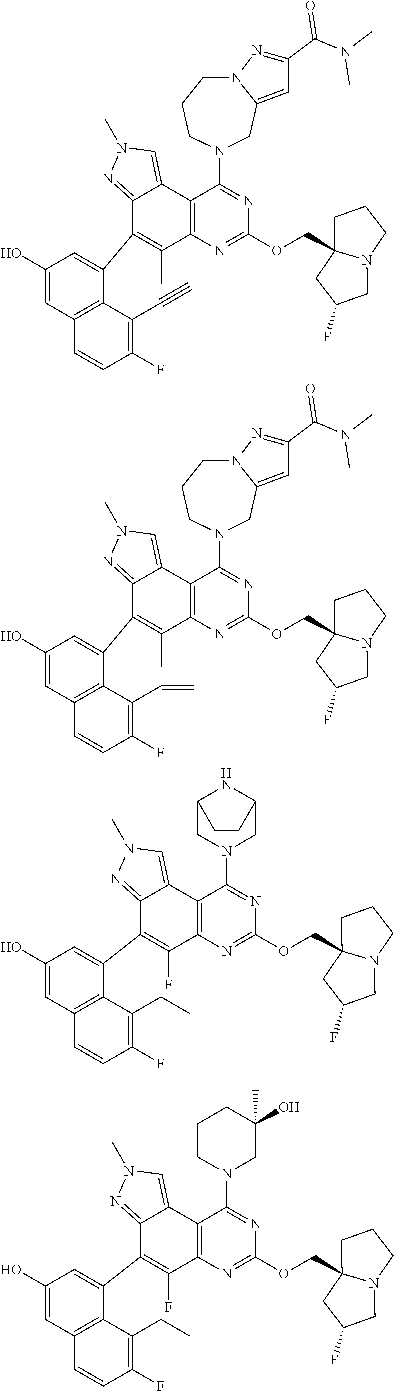 [SMILES: C#Cc1c(F)ccc2cc(O)cc(-c3c(C)c4nc(OC[C@@]56CCCN5C[C@H](F)C6)nc(N5CCCn6nc(C(=O)N(C)C)cc6C5)c4c4cn(C)nc34)c12 ; C=Cc1c(F)ccc2cc(O)cc(-c3c(C)c4nc(OC[C@@]56CCCN5C[C@H](F)C6)nc(N5CCCn6nc(C(=O)N(C)C)cc6C5)c4c4cn(C)nc34)c12 ; CCc1c(F)ccc2cc(O)cc(-c3c(F)c4nc(OC[C@@]56CCCN5C[C@H](F)C6)nc(N5CC6CCC(C5)N6)c4c4cn(C)nc34)c12 ; CCc1c(F)ccc2cc(O)cc(-c3c(F)c4nc(OC[C@@]56CCCN5C[C@H](F)C6)nc(N5CCC[C@@](C)(O)C5)c4c4cn(C)nc34)c12]

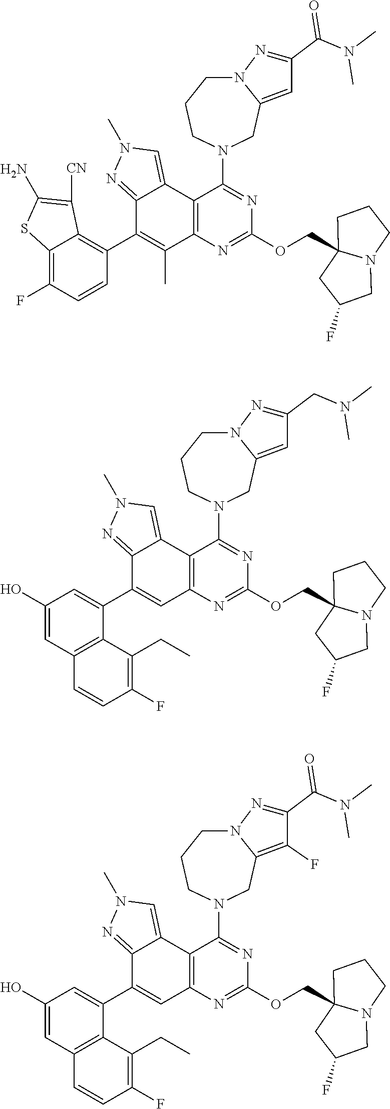 [SMILES: Cc1c(-c2ccc(F)c3sc(N)c(C#N)c23)c2nn(C)cc2c2c(N3CCCn4nc(C(=O)N(C)C)cc4C3)nc(OC[C@@]34CCCN3C[C@H](F)C4)nc12 ; CCc1c(F)ccc2cc(O)cc(-c3cc4nc(OC[C@@]56CCCN5C[C@H](F)C6)nc(N5CCCn6nc(CN(C)C)cc6C5)c4c4cn(C)nc34)c12 ; CCc1c(F)ccc2cc(O)cc(-c3cc4nc(OC[C@@]56CCCN5C[C@H](F)C6)nc(N5CCCn6nc(C(=O)N(C)C)c(F)c6C5)c4c4cn(C)nc34)c12]

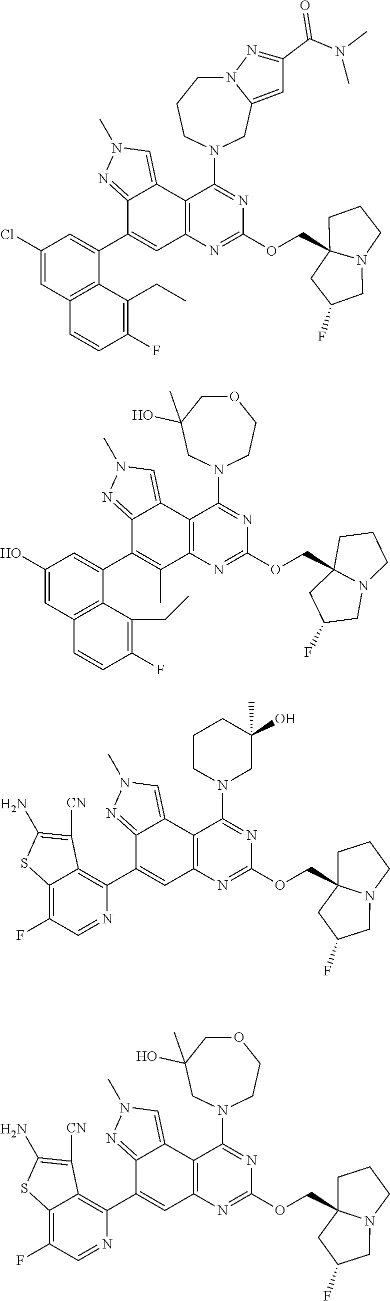

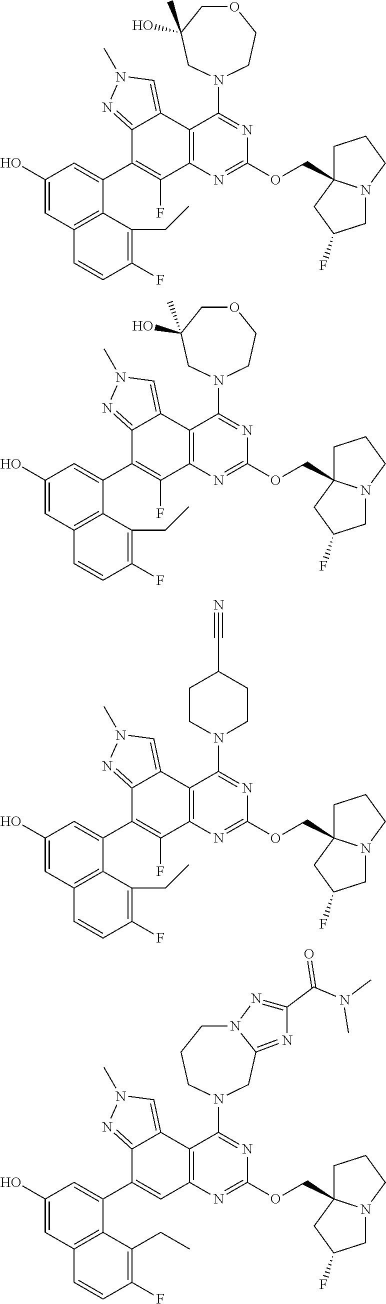

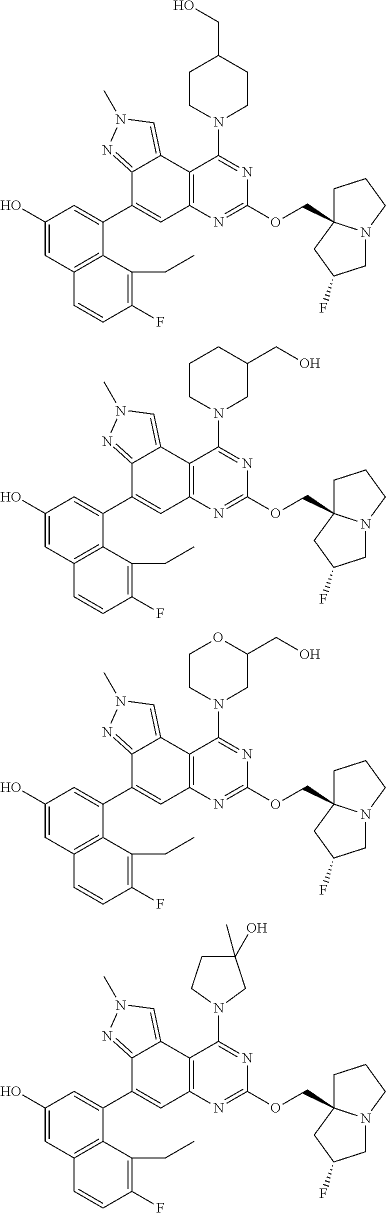 [SMILES: CCc1c(F)ccc2cc(O)cc(-c3cc4nc(OC[C@@]56CCCN5C[C@H](F)C6)nc(N5CCC(CO)CC5)c4c4cn(C)nc34)c12 ; CCc1c(F)ccc2cc(O)cc(-c3cc4nc(OC[C@@]56CCCN5C[C@H](F)C6)nc(N5CCCC(CO)C5)c4c4cn(C)nc34)c12 ; CCc1c(F)ccc2cc(O)cc(-c3cc4nc(OC[C@@]56CCCN5C[C@H](F)C6)nc(N5CCOC(CO)C5)c4c4cn(C)nc34)c12 ; CCc1c(F)ccc2cc(O)cc(-c3cc4nc(OC[C@@]56CCCN5C[C@H](F)C6)nc(N5CCC(C)(O)C5)c4c4cn(C)nc34)c12]

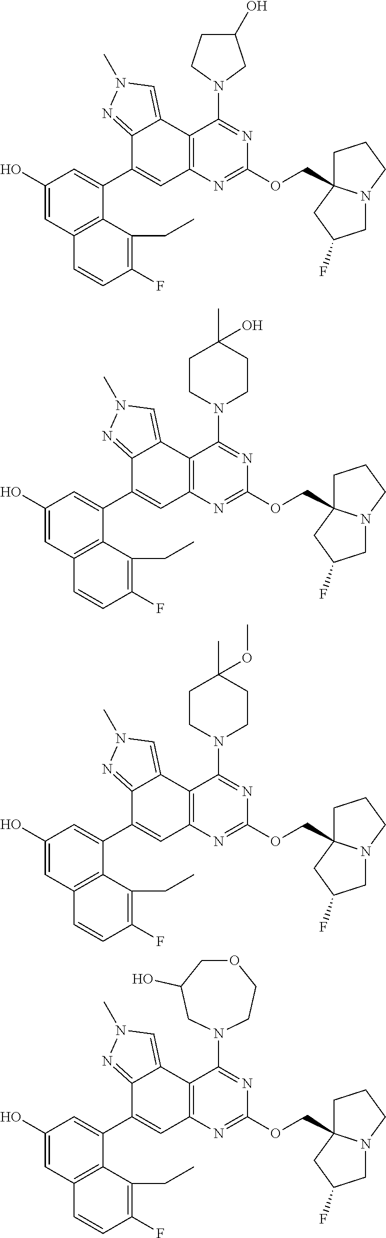

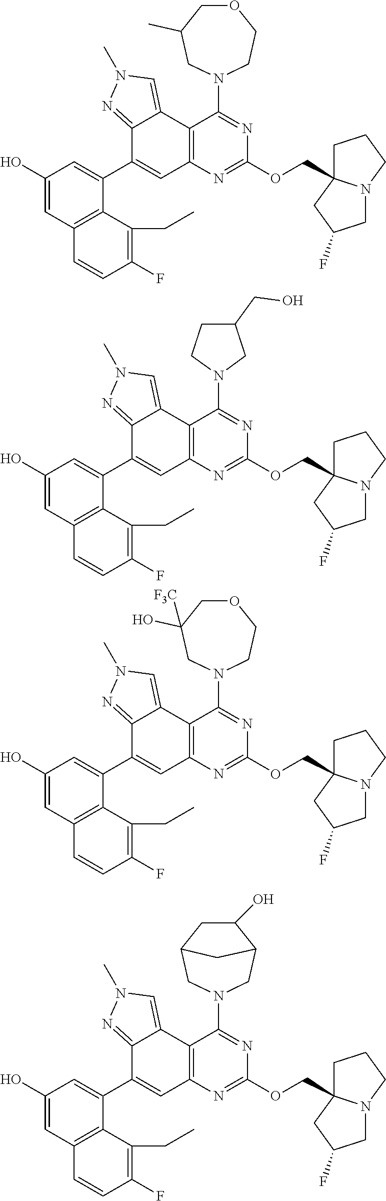

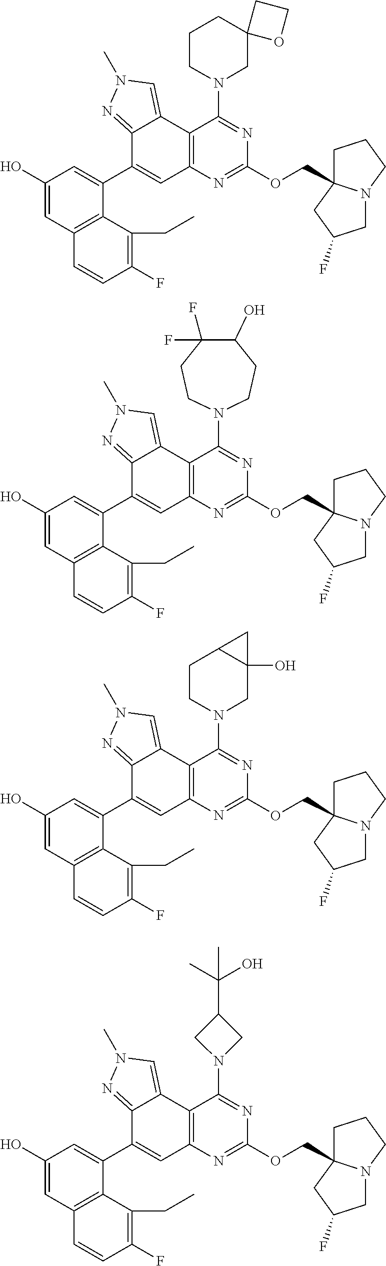

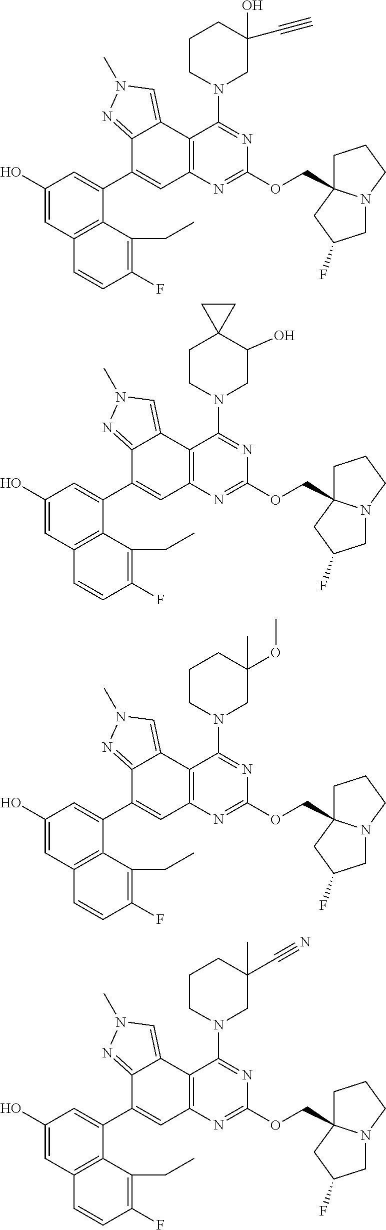

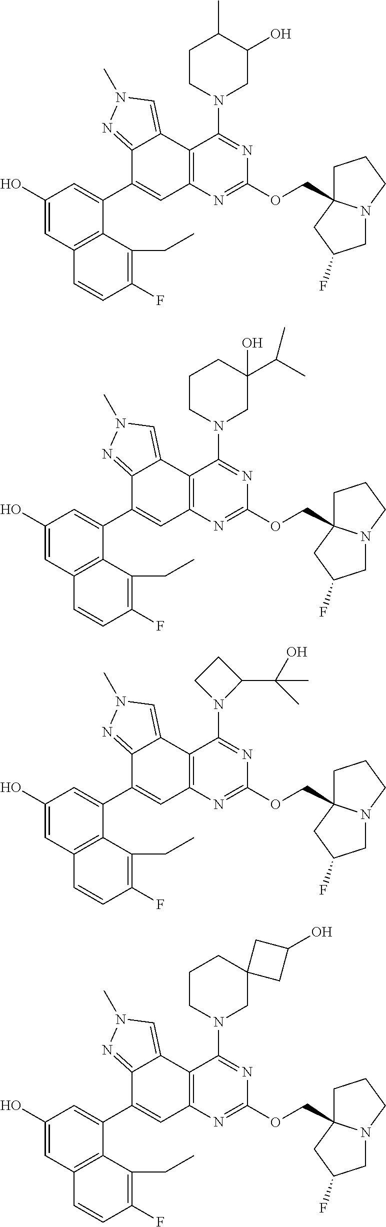

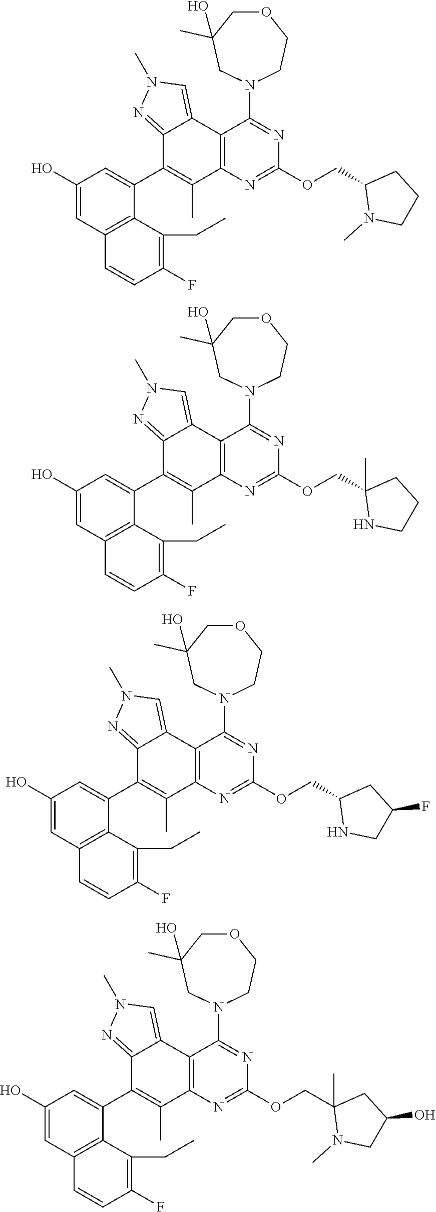

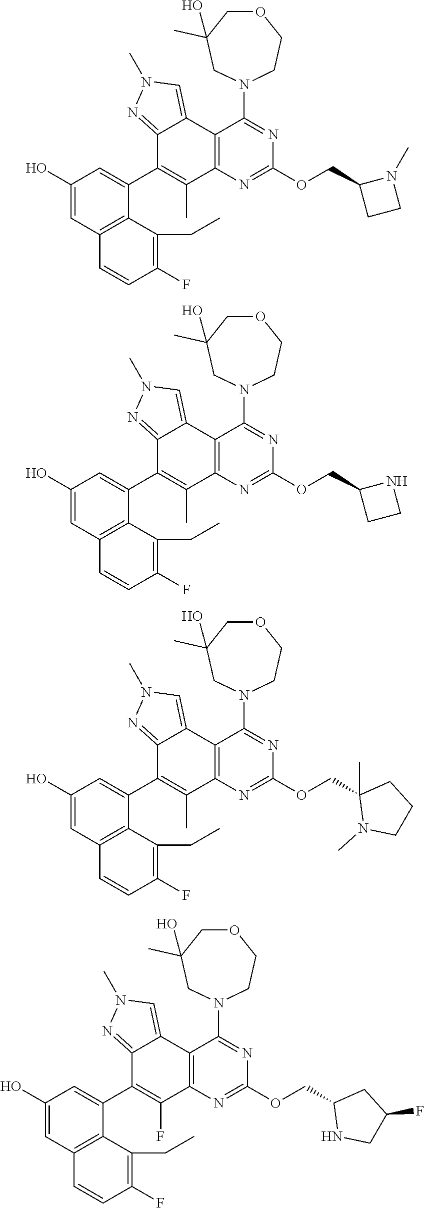

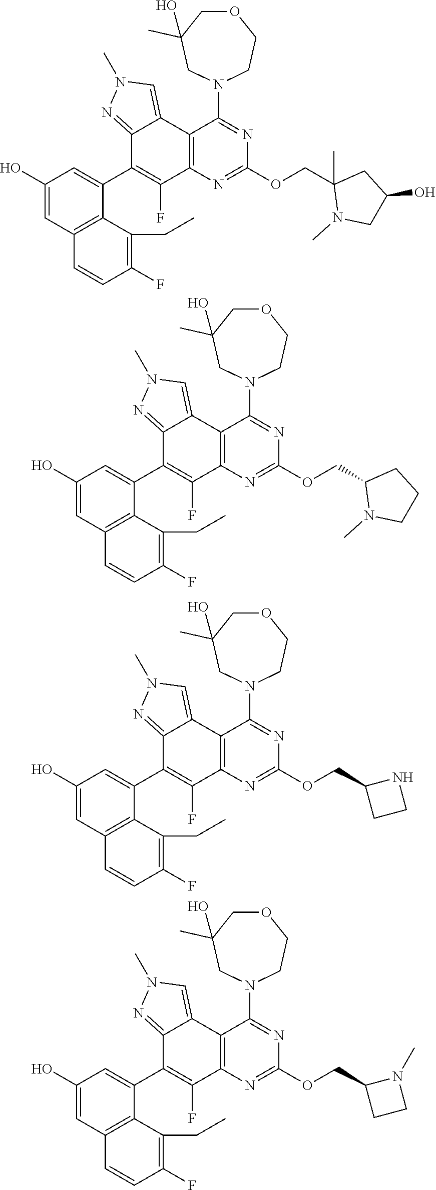

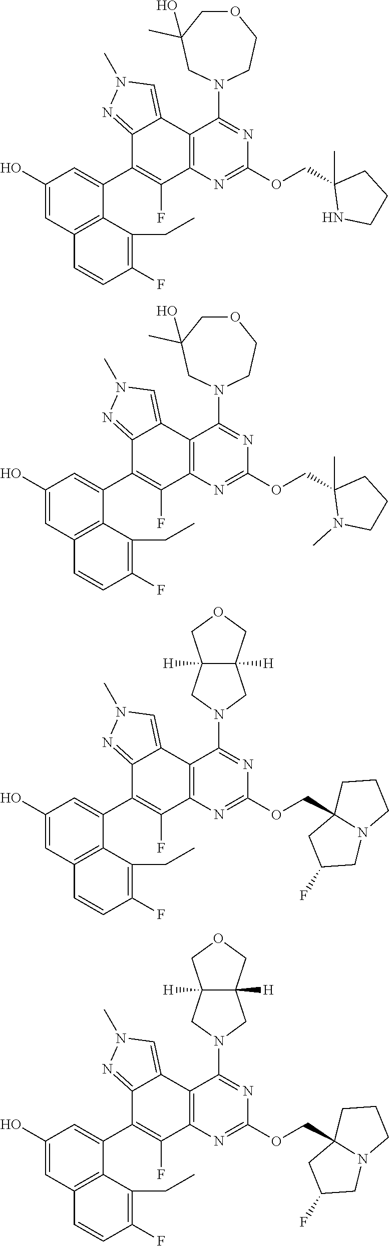

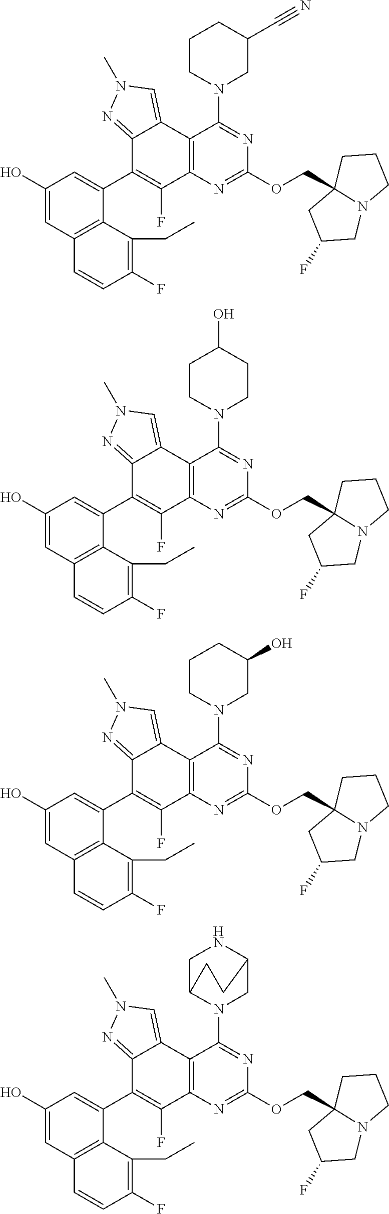 [SMILES: CCc1c(F)ccc2cc(O)cc(-c3c(F)c4nc(OC[C@@]56CCCN5C[C@H](F)C6)nc(N5CCCC(C#N)C5)c4c4cn(C)nc34)c12 ; CCc1c(F)ccc2cc(O)cc(-c3c(F)c4nc(OC[C@@]56CCCN5C[C@H](F)C6)nc(N5CCC(O)CC5)c4c4cn(C)nc34)c12 ; CCc1c(F)ccc2cc(O)cc(-c3c(F)c4nc(OC[C@@]56CCCN5C[C@H](F)C6)nc(N5CCC[C@@H](O)C5)c4c4cn(C)nc34)c12 ; CCc1c(F)ccc2cc(O)cc(-c3c(F)c4nc(OC[C@@]56CCCN5C[C@H](F)C6)nc(N5CC6CCC5CN6)c4c4cn(C)nc34)c12]

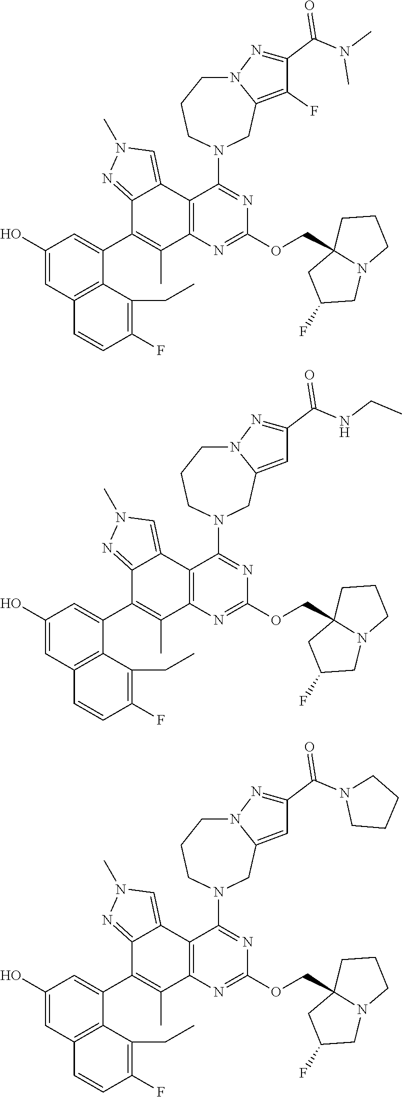

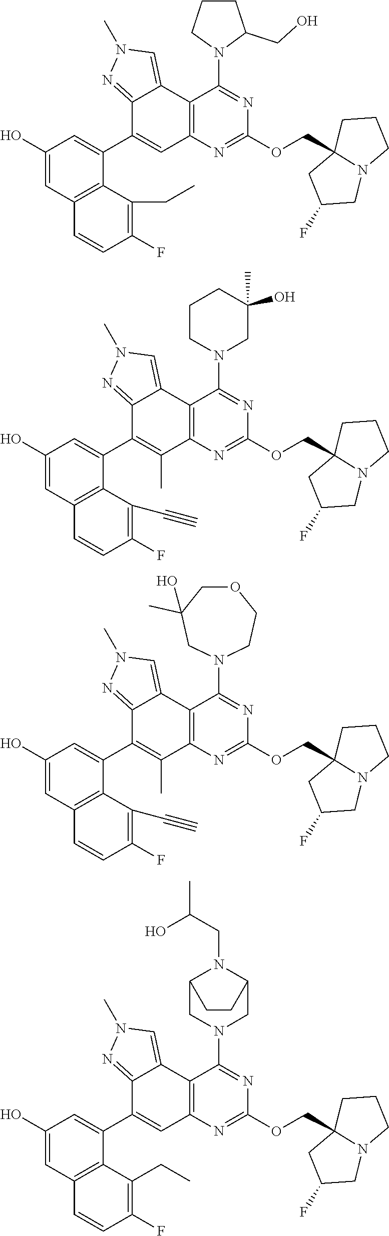

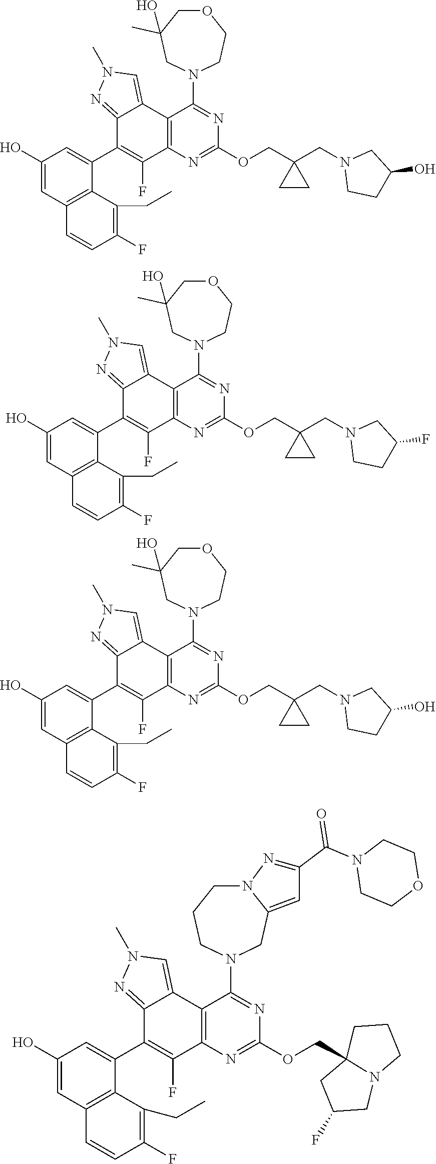

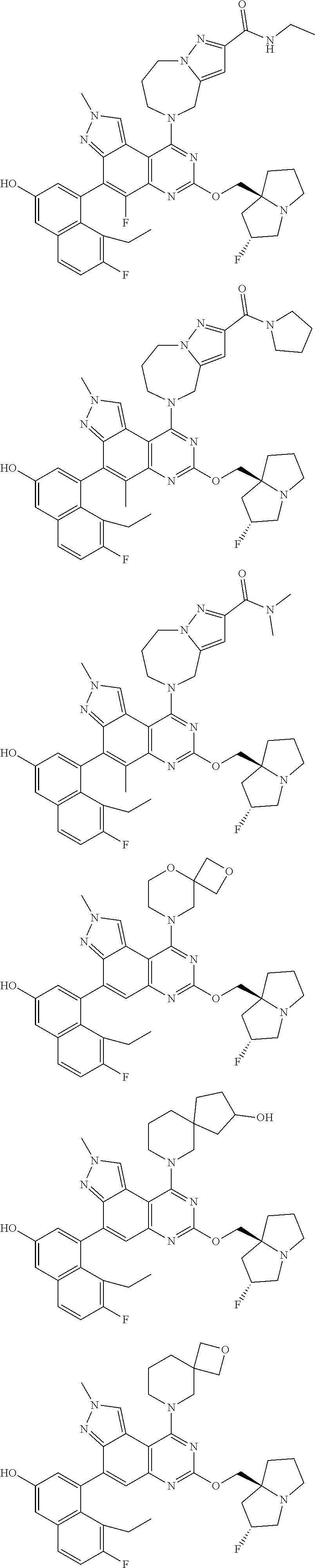

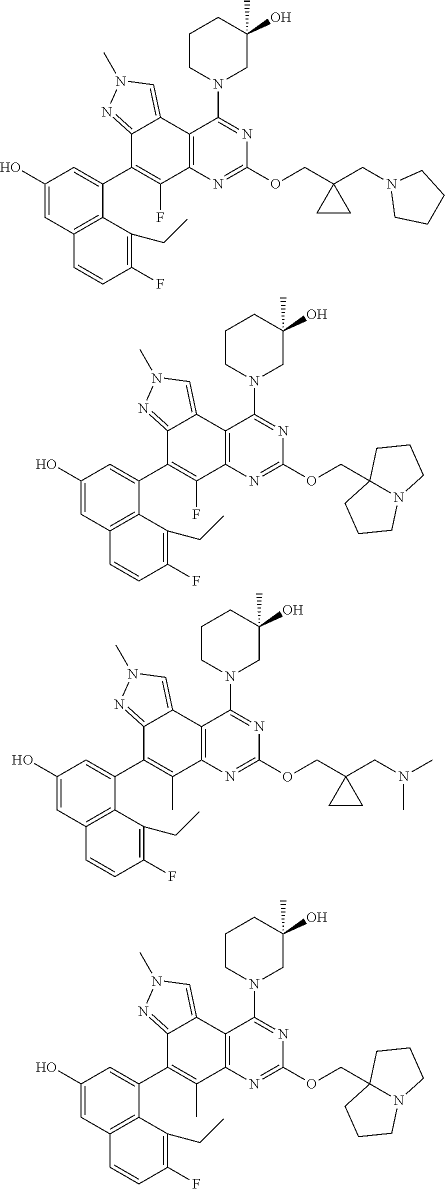

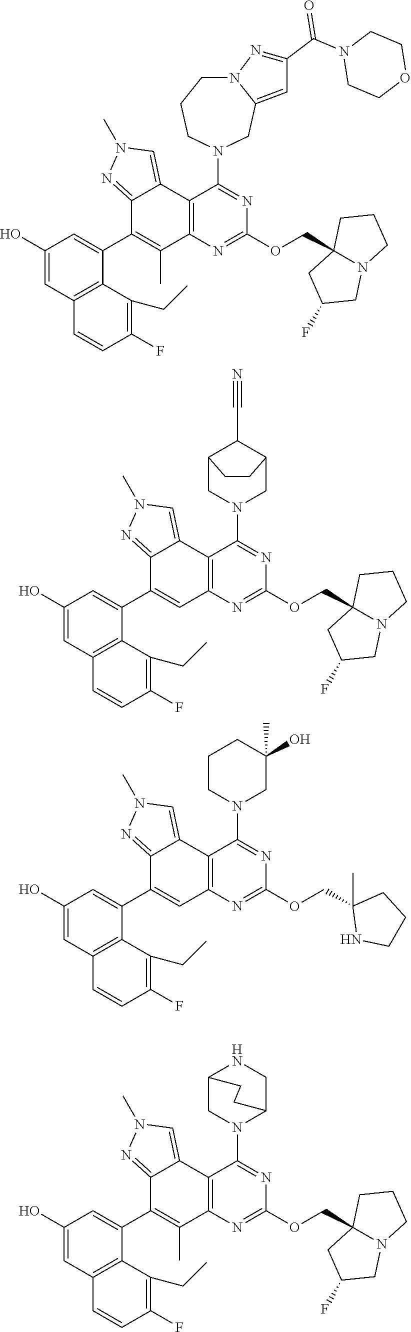 [SMILES: CCc1c(F)ccc2cc(O)cc(-c3c(C)c4nc(OC[C@@]56CCCN5C[C@H](F)C6)nc(N5CCCn6nc(C(=O)N7CCOCC7)cc6C5)c4c4cn(C)nc34)c12 ; CCc1c(F)ccc2cc(O)cc(-c3cc4nc(OC[C@@]56CCCN5C[C@H](F)C6)nc(N5CC6CCC(C5)C6C#N)c4c4cn(C)nc34)c12 ; CCc1c(F)ccc2cc(O)cc(-c3cc4nc(OC[C@]5(C)CCCN5)nc(N5CCC[C@@](C)(O)C5)c4c4cn(C)nc34)c12 ; CCc1c(F)ccc2cc(O)cc(-c3c(C)c4nc(OC[C@@]56CCCN5C[C@H](F)C6)nc(N5CC6CCC5CN6)c4c4cn(C)nc34)c12]

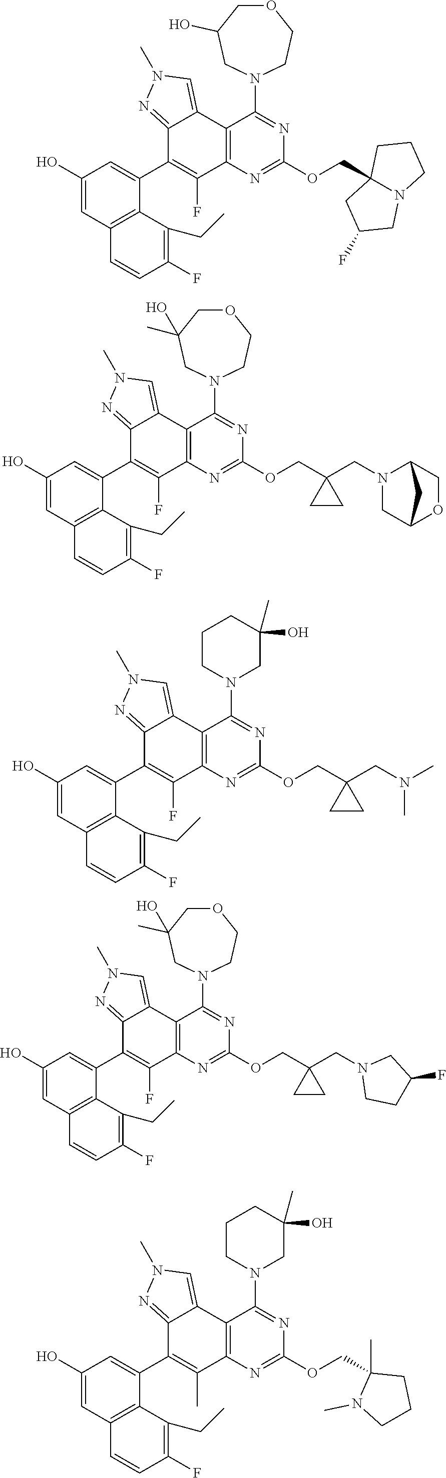

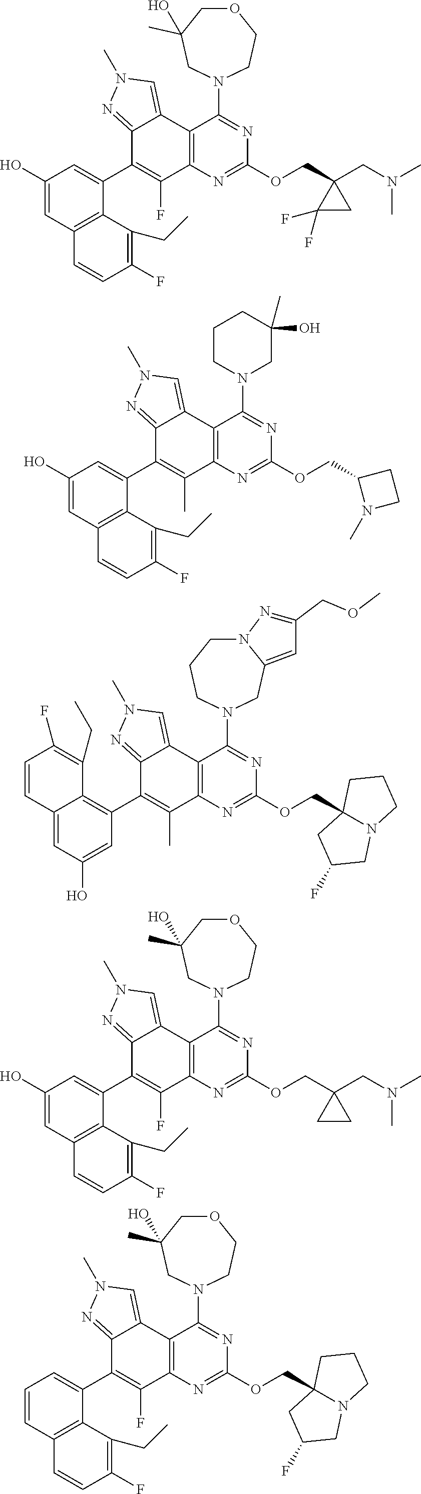

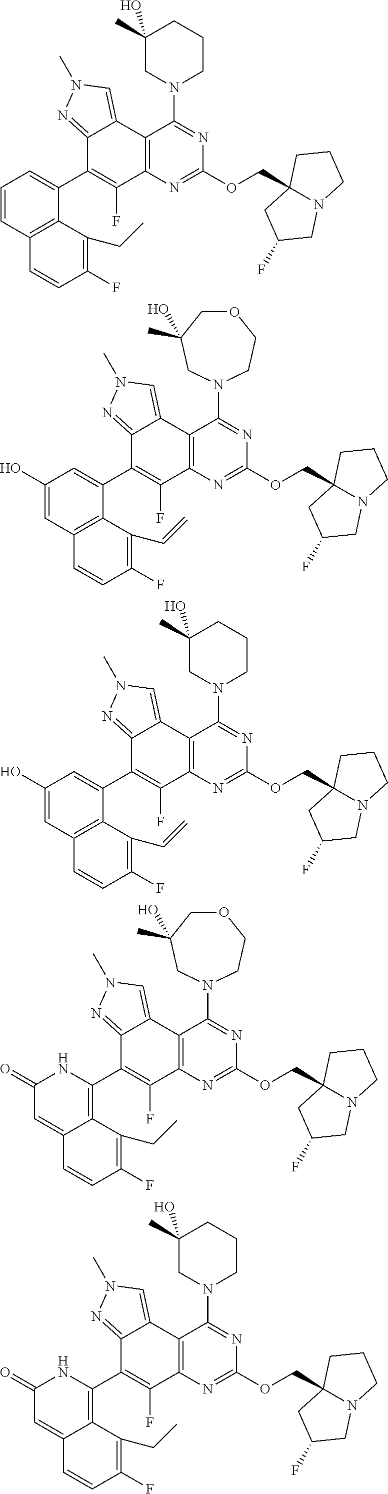

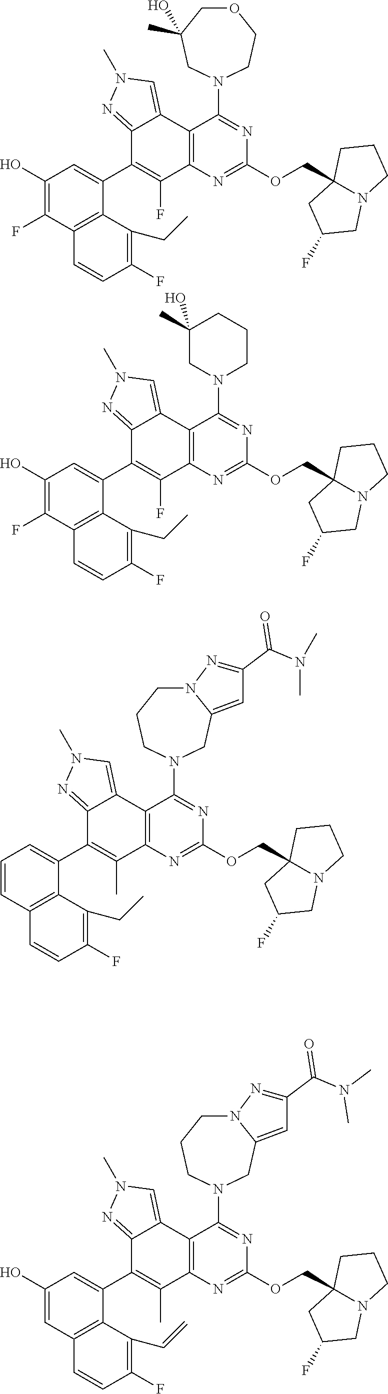

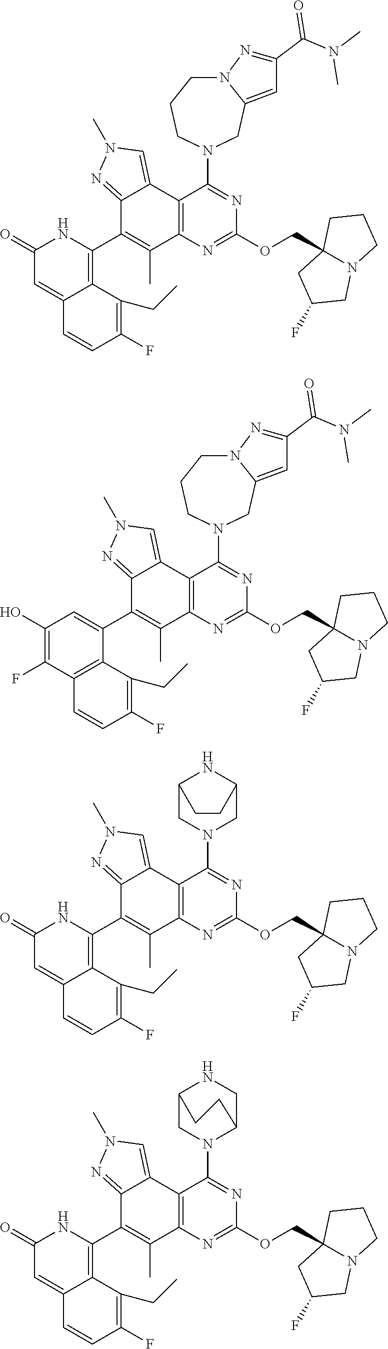 [SMILES: CCc1c(F)ccc2cc(=O)[nH]c(-c3c(C)c4nc(OC[C@@]56CCCN5C[C@H](F)C6)nc(N5CCCn6nc(C(=O)N(C)C)cc6C5)c4c4cn(C)nc34)c12 ; CCc1c(F)ccc2c(F)c(O)cc(-c3c(C)c4nc(OC[C@@]56CCCN5C[C@H](F)C6)nc(N5CCCn6nc(C(=O)N(C)C)cc6C5)c4c4cn(C)nc34)c12 ; CCc1c(F)ccc2cc(=O)[nH]c(-c3c(C)c4nc(OC[C@@]56CCCN5C[C@H](F)C6)nc(N5CC6CCC(C5)N6)c4c4cn(C)nc34)c12 ; CCc1c(F)ccc2cc(=O)[nH]c(-c3c(C)c4nc(OC[C@@]56CCCN5C[C@H](F)C6)nc(N5CC6CCC5CN6)c4c4cn(C)nc34)c12]

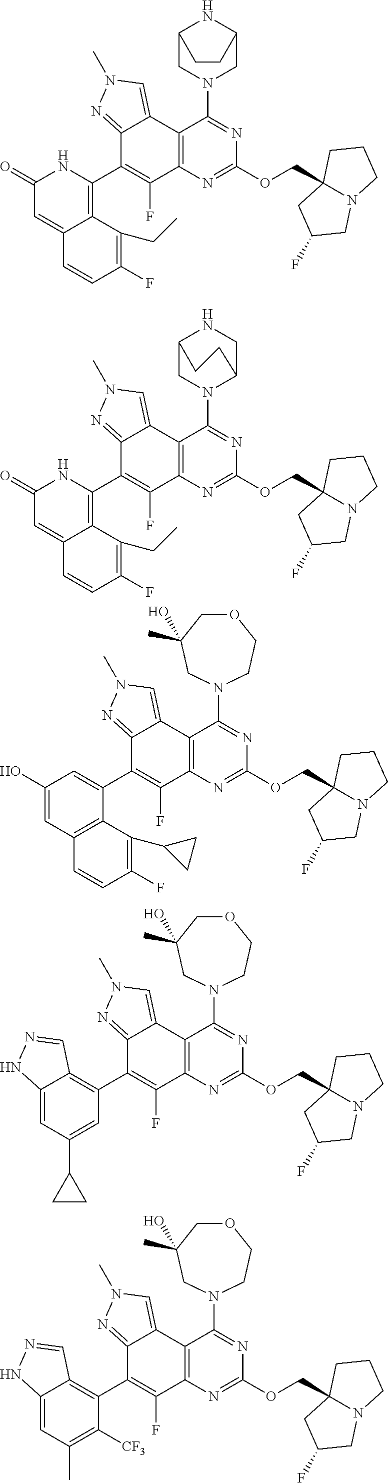

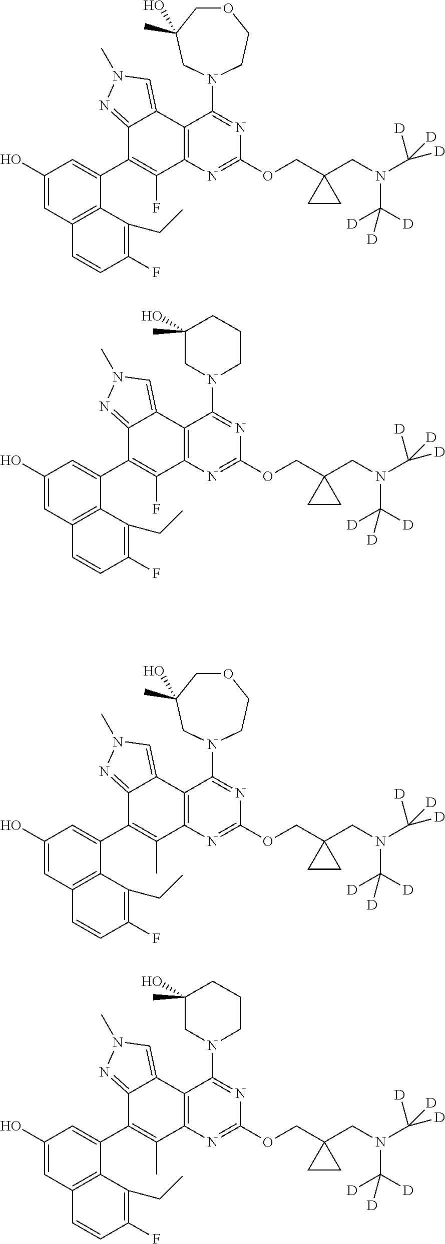

### DETAILED DESCRIPTION

In an aspect of the invention there is provided the compounds of the present invention for use as a medicament.

In accordance with another aspect, the present invention provides a method of treating a condition which can be modulated by inhibition of KRAS proteins having the G12D mutation, the method comprising administering a therapeutically effective amount of a compound of the invention to a subject in need thereof.

In accordance with another aspect, the present invention provides a pharmaceutical formulation comprising a compound of the present invention and a pharmaceutically acceptable excipient.

In an embodiment, the pharmaceutical composition may be a combination product comprising an additional pharmaceutically active agent. The additional pharmaceutically active agent may be, for example anti-inflammatory agents, anti-fibrotic agents, chemotherapeutics, anti-cancer agents, immunosuppressants, anti-tumour vaccines, cytokine therapy, or tyrosine kinase inhibitors.

In an aspect of the invention there is provided the compounds of the present invention for use in treating cancer.

In an aspect of the invention there is provided a method of treating cancer, the method comprising administering to a subject in need thereof a therapeutically effective amount of a compound of the invention.

In an aspect of the invention there is provided the use of a compound of the invention for manufacture of a medicament for the treatment of cancer.

The cancer may be a solid tumour or a liquid tumour. The cancer may be a carcinoma.

The cancer may be selected from cervical cancer, endometrial cancer, multiple myeloma, stomach cancer, bladder cancer, uterine cancer, esophageal squamous cell carcinoma, gastric cancer, glioblastomas, astrocytomas; retinoblastoma, osteosarcoma, chondosarcoma, Ewing's sarcoma, rabdomysarcoma, Wilm's tumor, basal cell carcinoma, non-small cell lung cancer, brain tumour, hormone refractory prostate cancer, prostate cancer, metastatic breast cancer, breast cancer, metastatic pancreatic cancer, pancreatic cancer, colorectal cancer, head and neck squamous cell carcinoma, cancer of the head and neck, appendix cancer, cholangiocarcinoma, cancer of unknown primary, ampulla of Vater cancer, ovarian cancer, acute myeloid leukaemia, small cell lung carcinoma, germ cell tumour, small bowel cancer, melanoma, soft tissue sarcoma, gastrointestinal stromal tumour, thyroid cancer, gastrointestinal neuroendocrine tumour, renal cell carcinoma and histiocytosis.

The cancer may be selected from pancreatic carcinoma, colorectal carcinoma, rectal carcinoma, endometrial carcinoma, non-small cell lung carcinoma, gastric carcinoma, ovarian carcinoma and small cell lung carcinoma.

The cancer may have wild-type KRAS. The cancer may have a KRAS mutation. The cancer may have a KRAS mutation selected from: KRAS G12D, KRAS G12C, KRAS G12V, KRAS G12A, KRAS G12S, KRAS G13D and KRAS Q61H. The cancer may have a KRAS G12D mutation. The cancer may have a KRAS G12D mutation, said cancer being selected from pancreatic carcinoma, colorectal carcinoma, rectal carcinoma, endometrial carcinoma, non-small cell lung carcinoma, gastric carcinoma, ovarian carcinoma and small cell lung carcinoma.

The cancer may have a confirmed KRAS G12D mutation. The cancer may have a confirmed KRAS G12D mutation, said cancer being selected from pancreatic carcinoma, colorectal carcinoma, rectal carcinoma, endometrial carcinoma, non-small cell lung carcinoma, gastric carcinoma, ovarian carcinoma and small cell lung carcinoma.

The subject may be human.

The subject may have a cancer with a KRAS G12D mutation. The subject may have a cancer with a KRAS G12D mutation, said cancer being selected from pancreatic carcinoma, colorectal carcinoma, rectal carcinoma, endometrial carcinoma, non-small cell lung carcinoma, gastric carcinoma, ovarian carcinoma and small cell lung carcinoma.

The subject may have a cancer with a confirmed KRAS G12D mutation. The subject may have a cancer with a confirmed KRAS G12D mutation, said cancer being selected from pancreatic carcinoma, colorectal carcinoma, rectal carcinoma, endometrial carcinoma, non-small cell lung carcinoma, gastric carcinoma, ovarian carcinoma and small cell lung carcinoma.

The subject may have a confirmed G12D mutation in their tumour. To be confirmed, the test for G12D presence in the tumour must have >95% for analytical specificity for the detection of mutations in the KRAS gene. Such validated tests would include already commercially available tests i.e. Foundation One CDx and CARIS DNA sequencing.

As mentioned above, the invention includes a method of treating cancer. The method may comprise:
a) confirming that the subject has a cancer with a G12D mutation; and
b) administering to a subject in need thereof a therapeutically effective amount of a compound of the invention.

The term “halo” refers to one of the halogens, group 17 of the periodic table. In particular the term refers to fluorine, chlorine, bromine and iodine. Preferably, the term refers to fluorine or chlorine.

The term “alkyl” refers to a linear or branched hydrocarbon chain. For example, the term “C₁₋₆ alkyl” or “C₁₋₄-alkyl” refers to a linear or branched hydrocarbon chain containing 1, 2, 3, 4, 5, or 6 carbon atoms, for example methyl, ethyl, n-propyl, iso-propyl, n-butyl, sec-butyl, tert-butyl, n-pentyl and n-hexyl. Where an alkyl group is indicated as being C₀₋₄alkyl, then it should be appreciated that this represents the possibility for the alkyl unit to be absent or 1, 2, 3, or 4 carbon atoms in length. Alkylene groups may likewise be linear or branched and may have two places of attachment to the remainder of the molecule. Furthermore, an alkylene group may, for example, correspond to one of those alkyl groups listed in this paragraph. The alkyl and alkylene groups may be unsubstituted or substituted by one or more substituents. Possible substituents are described below. Substituents for the alkyl group may be halogen, e.g. fluorine, chlorine, bromine and iodine, OH, C₁₋₆ alkoxy.

The term “alkoxy” refers to an alkyl group which is attached to a molecule via oxygen. For example, the term “C₁₋₆ alkoxy” refers to an alkyl group which is attached to a molecule via oxygen. This includes moieties where the alkyl part may be linear or branched and may contain 1, 2, 3, 4, 5, or 6 carbon atoms, for example methyl, ethyl, n-propyl, iso-propyl, n-butyl, sec-butyl, tert-butyl, n-pentyl and n-hexyl. Therefore, the alkoxy group may be methoxy, ethoxy, n-propoxy, iso-propoxy, n-butoxy, sec-butoxy, tert-butoxy, n-pentoxy and n-hexoxy. The alkyl part of the alkoxy group may be unsubstituted or substituted by one or more substituents. Possible substituents are described below. Substituents for the alkyl group may be halogen, e.g. fluorine, chlorine, bromine and iodine, OH, C₁₋₆ alkoxy.

The term “haloalkyl” refers to a hydrocarbon chain substituted with at least one halogen atom independently chosen at each occurrence, for example fluorine, chlorine, bromine and iodine. For example, the term “C₁₋₆ haloalkyl” refers to a linear or branched hydrocarbon chain containing 1, 2, 3, 4, 5 or 6 carbon atoms substituted with at least one halogen. The halogen atom may be present at any position on the hydrocarbon chain. For example, C₁₋₆ haloalkyl may refer to chloromethyl, fluoromethyl, trifluoromethyl, chloroethyl e.g. 1-chloromethyl and 2-chloroethyl, trichloroethyl e.g. 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl.

The term “alkenyl” refers to a branched or linear hydrocarbon chain containing at least one double bond. For example, the term “C₂₋₆ alkenyl” refers to a branched or linear hydrocarbon chain containing at least one double bond and having 2, 3, 4, 5 or 6 carbon atoms. The double bond(s) may be present as the E or Z isomer. The double bond may be at any possible position of the hydrocarbon chain. For example, the “C₂₋₆ alkenyl” may be ethenyl, propenyl, butenyl, butadienyl, pentenyl, pentadienyl, hexenyl and hexadienyl.

The term “alkynyl” refers to a branched or linear hydrocarbon chain containing at least one triple bond. For example, the term “C₂₋₆ alkynyl” refers to a branched or linear hydrocarbon chain containing at least one triple bond and having 2, 3, 4, 5 or 6 carbon atoms. The triple bond may be at any possible position of the hydrocarbon chain. For example, the “C₂₋₆ alkynyl” may be ethynyl, propynyl, butynyl, pentynyl and hexynyl.

The term “heteroalkyl” refers to a branched or linear hydrocarbon chain containing at least one heteroatom selected from N, O and S positioned between any carbon in the chain or at an end of the chain. For example, the term “C₁₋₆ heteroalkyl” refers to a branched or linear hydrocarbon chain containing 1, 2, 3, 4, 5, or 6 carbon atoms and at least one heteroatom selected from N, O and S positioned between any carbon in the chain or at an end of the chain. For example, the hydrocarbon chain may contain one or two heteroatoms. The C₁₋₆ heteroalkyl may be bonded to the rest of the molecule through a carbon or a heteroatom. For example, the “C₁₋₆ heteroalkyl” may be C₁₋₆ N-alkyl, C₁₋₆ N,N-alkyl, or C₁₋₆ O-alkyl.

The term “cycloalkyl” refers to a saturated hydrocarbon ring system. For example, the “C₃₋₈ cycloalkyl” may be cyclopropyl, cyclobutyl, cyclopentyl, cyclohexyl, cycloheptyl and cyclooctyl.

The term “cycloalkenyl” refers to an unsaturated hydrocarbon ring system containing that is not aromatic. The ring may contain more than one double bond provided that the ring system is not aromatic. For example, the “C₃₋₈ cycloalkyl” may be cyclopropenyl, cyclobutenyl, cyclopentenyl, cyclopentadienyl, I, cyclohexenyl, cyclohexadienyl, cycloheptenyl, cycloheptadiene, cyclooctenyl and cycloatadienyl.

The term “heterocycloalkyl” refers to a saturated hydrocarbon ring system containing carbon atoms and at least one heteroatom within the ring selected from N, O and S. For example, there may be 1, 2 or 3 heteroatoms, optionally 1 or 2. The “heterocycloalkyl” may be bonded to the rest of the molecule through any carbon atom or heteroatom. For example, the “heterocycloalkyl” may be a “C₃₋₈ heterocycloalkyl”. The term “C₃₋₈ heterocycloalkyl” refers to a saturated hydrocarbon ring system containing 3, 4, 5, 6, 7 or 8 carbon atoms and at least one heteroatom within the ring selected from N, O and S. For example, there may be 1, 2 or 3 heteroatoms, optionally 1 or 2. The “C₃₋₈ heterocycloalkyl” may be bonded to the rest of the molecule through any carbon atom or heteroatom. A “heterocycloakyl” group may be monocyclic. A “heterocycloakyl” group may be bicyclic e.g., a fused, spirofused or bridged heterocycloalkyl ring system. For example, the “C₃₋₈ heterocycloalkyl” may be oxirane, aziridine, azetidine, oxetane, tetrahydrofuran, pyrrolidine, imidazolidine, succinimide, pyrazolidine, oxazolidine, isoxazolidine, thiazolidine, isothiazolidine, piperidine, morpholine, thiomorpholine, piperazine, and tetrahydropyran.

The term “heterocyclyl” refers to a saturated or unsaturated hydrocarbon ring system containing carbon atoms and at least one heteroatom within the ring selected from N, O and S. For example, there may be 1, 2 or 3 heteroatoms, optionally 1 or 2. The “heterocyclyl” may be bonded to the rest of the molecule through any carbon atom or heteroatom. The “heterocyclyl” may be a heterocycloalkyl. The “heterocyclyl” may be a heteroaryl. The “heterocyclyl” may be a bicyclic heterocyclyl, e.g. a bicyclic heteroaryl or a bicyclic heterocycloalkyl. The “heterocyclyl” may be a heterocycloalkyl ring fused to an aryl or heteroaryl ring. The “heterocyclyl” may be a heteroaryl ring fused to an cycloalkyl or heterocycloalkyl ring. The “heterocyclyl” may be a monocyclic heterocyclyl, e.g. a monocyclic heteroaryl, a monocyclic heterocycloalkyl.

The term “heterocycloalkenyl” refers to an unsaturated hydrocarbon ring system that is not aromatic, containing carbon atoms and at least one heteroatom within the ring selected from N, O and S. For example, there may be 1, 2 or 3 heteroatoms, optionally 1 or 2. The “heterocycloalkenyl” may be bonded to the rest of the molecule through any carbon atom or heteroatom. For example, the “heterocycloalkenyl” may be a “C₃₋₈ heterocycloalkenyl”. The term “C₃₋₈ heterocycloalkenyl” refers to a saturated hydrocarbon ring system containing 3, 4, 5, 6, 7 or 8 atoms at least one of the atoms being a heteroatom within the ring selected from N, O and S. The “heterocycloalkenyl” may be tetrahydropyridine, dihydropyran, dihydrofuran, pyrroline.

The term “fused” refers to a bicyclic ring system in which the two rings are attached via two atoms that are situated adjacent to each other on each ring.

The term “spirofused” refers to a bicyclic ring system in which the two rings are attached via a single atom.

The term “bridged” refers to a bicyclic ring system in which the two rings are attached via two atoms that are not situated adjacent to each other on either ring.

The term “aromatic” when applied to a substituent as a whole means a single ring or polycyclic ring system with 4n+2 electrons in a conjugated IT system within the ring or ring system where all atoms contributing to the conjugated IT system are in the same plane.

The term “aryl” refers to an aromatic hydrocarbon ring system. The ring system has 4n+2 electrons in a conjugated IT system within a ring where all atoms contributing to the conjugated TT system are in the same plane. For example, the “aryl” may be phenyl and naphthyl. The aryl system itself may be substituted with other groups.

The term “heteroaryl” refers to an aromatic hydrocarbon ring system with at least one heteroatom within a single ring or within a fused ring system, selected from O, N and S. The ring or ring system has 4n+2 electrons in a conjugated IT system where all atoms contributing to the conjugated IT system are in the same plane. For example, the “heteroaryl” may be imidazole, thiene, furane, thianthrene, pyrrole, benzimidazole, pyrazole, pyrazine, pyridine, pyrimidine and indole.

The term “halogen” herein includes reference to F, Cl, Br and I. Halogen may be Br. Halogen may be I.

A bond terminating in a “ [symbol] ” represents that the bond is connected to another atom that is not shown in the structure. A bond terminating inside a cyclic structure and not terminating at an atom of the ring structure represents that the bond may be connected to any of the atoms in the ring structure where allowed by valency.

Where a moiety is substituted, it may be substituted at any point on the moiety where chemically possible and consistent with atomic valency requirements. The moiety may be substituted by one or more substituents, e.g. 1, 2, 3 or 4 substituents; optionally there are 1 or 2 substituents on a group. Where there are two or more substituents, the substituents may be the same or different.

Substituents are only present at positions where they are chemically possible, the person skilled in the art being able to decide (either experimentally or theoretically) without inappropriate effort which substitutions are chemically possible, and which are not.

Ortho, meta and para substitution are well understood terms in the art. For the absence of doubt, “ortho” substitution is a substitution pattern where adjacent carbons possess a substituent, whether a simple group, for example the fluoro group in the example below, or other portions of the molecule, as indicated by the bond ending in “ [symbol] ”.

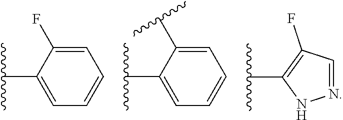

“Meta” substitution is a substitution pattern where two substituents are on carbons one carbon removed from each other, i.e. with a single carbon atom between the substituted carbons. In other words, there is a substituent on the second atom away from the atom with another substituent. For example, the groups below are meta substituted.

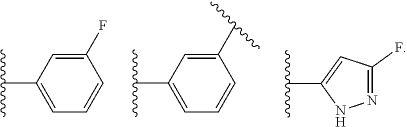

“Para” substitution is a substitution pattern where two substituents are on carbons two carbons removed from each other, i.e with two carbon atoms between the substituted carbons. In other words, there is a substituent on the third atom away from the atom with another substituent. For example, the groups below are para substituted.

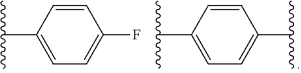

Throughout the description the disclosure of a compound also encompasses pharmaceutically acceptable salts, solvates and stereoisomers thereof. Where a compound has a stereocentre, both (R) and(S) stereoisomers are contemplated by the invention, equally mixtures of stereoisomers or a racemic mixture are completed by the present application. Where a compound of the invention has two or more stereocentres any combination of (R) and(S) stereoisomers is contemplated. The combination of (R) and(S) stereoisomers may result in a diastereomeric mixture or a single diastereoisomer. The compounds of the invention may be present as a single stereoisomer or may be mixtures of stereoisomers, for example racemic mixtures and other enantiomeric mixtures, and diasteroemeric mixtures. Where the mixture is a mixture of enantiomers the enantiomeric excess may be any of those disclosed above. Where the compound is a single stereoisomer the compounds may still contain other diasteroisomers or enantiomers as impurities. Hence a single stereoisomer does not necessarily have an enantiomeric excess (e.e.) or diastereomeric excess (d.e.) of 100% but could have an e.e. or d.e. of about at least 85%, at least 60% or less. For example, the e.e. or d.e. may be 90% or more, 90% or more, 80% or more, 70% or more, 60% or more, 50% or more, 40% or more, 30% or more, 20% or more, or 10% or more.

The invention contemplates pharmaceutically acceptable salts of the compounds of the invention. These may include the acid addition and base salts of the compounds. These may be acid addition and base salts of the compounds. In addition, the invention contemplates solvates of the compounds. These may be hydrates or other solvated forms of the compound.

Suitable acid addition salts are formed from acids which form non-toxic salts. Examples include the acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 1,5-naphthalenedisulfonate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts. For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of formula (I) may be prepared by one or more of three methods:
(i) by reacting the compound of the invention with the desired acid or base;
(ii) by removing an acid- or base-labile protecting group from a suitable precursor of the compound of the invention or by ring-opening a suitable cyclic precursor, for example, a lactone or lactam, using the desired acid or base; or
(iii) by converting one salt of the compound of the invention to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in both unsolvated and solvated forms.

The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

Hereinafter all references to compounds of any formula include references to salts, solvates and complexes thereof and to solvates and complexes of salts thereof.

The compounds of the invention include compounds of a number of formulae as herein defined, including all polymorphs and crystal habits thereof, prodrugs and isomers thereof (including optical, geometric and tautomeric isomers) as hereinafter defined and isotopically-labelled compounds of the invention.

The present invention also includes all pharmaceutically acceptable isotopically-labelled compounds of the invention wherein one or more atoms are replaced by atoms having the same atomic number, but an atomic mass or mass number different from the atomic mass or mass number most commonly found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Before purification, the compounds of the present invention may exist as a mixture of enantiomers depending on the synthetic procedure used. The enantiomers can be separated by conventional techniques known in the art. Thus the invention covers individual enantiomers as well as mixtures thereof.

For some of the steps of the process of preparation of the compounds of the invention, it may be necessary to protect potential reactive functions that are not wished to react, and to cleave said protecting groups in consequence. In such a case, any compatible protecting radical can be used. In particular methods of protection and deprotection such as those described by T.W. GREENE (Protective Groups in Organic Synthesis, A. Wiley-Interscience Publication, 1981) or by P. J. Kocienski (Protecting groups, Georg Thieme Verlag, 1994), can be used. All of the above reactions and the preparations of novel starting materials used in the preceding methods are conventional and appropriate reagents and reaction conditions for their performance or preparation as well as procedures for isolating the desired products will be well-known to those skilled in the art with reference to literature precedents and the examples and preparations hereto.

Also, the compounds of the present invention as well as intermediates for the preparation thereof can be purified according to various well-known methods, such as for example crystallization or chromatography.

One or more compounds of the invention may be combined with one or more pharmaceutical agents, for example anti-inflammatory agents, anti-fibrotic agents, chemotherapeutics, anti-cancer agents, immunosuppressants, anti-tumour vaccines, cytokine therapy, or tyrosine kinase inhibitors, for the treatment of conditions modulated by the inhibition of RAS proteins, for example cancer, sarcoma, melanoma, skin cancer, haematological tumors, lymphoma, carcinoma, and leukemia.

The method of treatment or the compound for use in the treatment of cancer, sarcoma, melanoma, skin cancer, haematological tumors, lymphoma, carcinoma, and leukemia as defined hereinbefore may be applied as a sole therapy or be a combination therapy with an additional active agent.

The method of treatment or the compound for use in the treatment of cancer, sarcoma, melanoma, skin cancer, haematological tumors, lymphoma, carcinoma, and leukemia may involve, in addition to the compound of the invention, additional active agents. The additional active agents may be one or more active agents used to treat the condition being treated by the compound of the invention and additional active agent. The additional active agents may include one or more of the following active agents:
(i) steroids such as corticosteroids, including glucocorticoids and mineralocorticoids, for example aclometasone, aclometasone dipropionate, aldosterone, amcinonide, beclomethasone, beclomethasone dipropionate, betamethasone, betamethasone dipropionate, betamethasone sodium phosphate, betamethasone valerate, budesonide, clobetasone, clobetasone butyrate, clobetasol propionate, cloprednol, cortisone, cortisone acetate, cortivazol, deoxycortone, desonide, desoximetasone, dexamethasone, dexamethasone sodium phosphate, dexamethasone isonicotinate, difluorocortolone, fluclorolone, flumethasone, flunisolide, fluocinolone, fluocinolone acetonide, fluocinonide, fluocortin butyl, fluorocortisone, fluorocortolone, fluocortolone caproate, fluocortolone pivalate, fluorometholone, fluprednidene, fluprednidene acetate, flurandrenolone, fluticasone, fluticasone propionate, halcinonide, hydrocortisone, hydrocortisone acetate, hydrocortisone butyrate, hydrocortisone aceponate, hydrocortisone buteprate, hydrocortisone valerate, icomethasone, icomethasone enbutate, meprednisone, methylprednisolone, mometasone paramethasone, mometasone furoate monohydrate, prednicarbate, prednisolone, prednisone, tixocortol, tixocortol pivalate, triamcinolone, triamcinolone acetonide, triamcinolone alcohol and their respective pharmaceutically acceptable derivatives. A combination of steroids may be used, for example a combination of two or more steroids mentioned in this paragraph;
(ii) TNF inhibitors for example etanercept; monoclonal antibodies (e.g. infliximab (Remicade), adalimumab (Humira), certolizumab pegol (Cimzia), golimumab (Simponi)); fusion proteins (e.g. etanercept (Enbrel)); and 5-HT2A agonists (e.g. 2,5-dimethoxy-4-iodoamphetamine, TCB-2, lysergic acid diethylamide (LSD), lysergic acid dimethylazetidide);
(iii) anti-inflammatory drugs, for example non-steroidal anti-inflammatory drugs;
(iv) dihydrofolate reductase inhibitors/antifolates, for example methotrexate, trimethoprim, brodimoprim, tetroxoprim, iclaprim, pemetrexed, ralitrexed and pralatrexate; and
(v) immunosuppressants for example cyclosporins, tacrolimus, sirolimus pimecrolimus, angiotensin II inhibitors (e.g. Valsartan, Telmisartan, Losartan, Irbesatan, Azilsartan, Olmesartan, Candesartan, Eprosartan) and ACE inhibitors e.g. sulfhydryl-containing agents (e.g. Captopril, Zofenopril), dicarboxylate-containing agents (e.g. Enalapril, Ramipril, Quinapril, Perindopril, Lisinopril, Benazepril, Imidapril, Zofenopril, Trandolapril), phosphate-containing agents (e.g. Fosinopril), casokinins, lactokinins and lactotripeptides.
(vi) Anti-fibrotic agents for example: Pirfenidone, Nintedanib, Anti-IL-13 monoclonal antibodies (e.g. Tralokinumab, QAX576, Lebrikizumab), simtuzumab, FG-3019, lysophosphatidic acid receptor antagonists (e.g. BMS-986020, AM966), LOXL2 inhibitors, BET bromodomain inhibitors (e.g. JQ1), HDAC inhibitors (e.g. Vorinostat), thrombin inhibitors (e.g. Dabigatran), FactorXa inhibitors (e.g. Apixban, Rivaroxaban) 15PGDH inhibitors, anti-avβ6 monoclonal antibodies (e.g. BG00011), Anti-CTGF monoclonal antibodies (e.g. FG-3019), PAR1 inhibitors, Nox4 inhibitors and PAI-1 inhibitors.
(vii) CNS therapies, for example: Levodopa, Dopamine agonists, Apomorphine, Glutamate antagonist, Anticholinergics, COMT inhibitors, MAO-B inhibitors, riluzole (Rilutek), Tetrabenazine (Xenazine), haloperidol (Haldol), chlorpromazine, risperidone (Risperdal), quetiapine (Seroquel), amantadine, levetiracetam (Keppra), clonazepam (Klonopin), Donepezil (Aricept), Galantamine (Razadyne), Rivastigmine (Exelon)), Memantine (Ebixa, Axura), Aducanumab, Ocrelizumab, interferon beta-1a (Avonex, Rebif), peginterferon beta-1a (Plegridy), teriflunomide (Aubagio), fingolimod (Gilenya), mitoxantrone (Novantrone), dimethyl fumarate (Tecfidera), natalizumab (Tysabri)

The method of treatment or the compound for use in the treatment of cancer, sarcoma, melanoma, skin cancer, haematological tumors, lymphoma, carcinoma, leukemia, and central nervous system disorders may involve, in addition to the compound of the invention, conventional surgery or radiotherapy or chemotherapy. Such chemotherapy may include one or more of the following categories of anti-tumor agents:
(i) antiproliferative/antineoplastic drugs and combinations thereof, such as alkylating agents (for example cis platin, oxaliplatin, carboplatin, cyclophosphamide, nitrogen mustard, uracil mustard, bendamustin, melphalan, chlorambucil, chlormethine, busulphan, temozolamide, nitrosoureas, ifosamide, melphalan, pipobroman, triethylene-melamine, triethylenethiophoporamine, carmustine, lomustine, stroptozocin and dacarbazine); antimetabolites (for example gemcitabine and antifolates such as fluoropyrimidines like 5 fluorouracil and tegafur, raltitrexed, methotrexate, pemetrexed, cytosine arabinoside, floxuridine, cytarabine, 6-mercaptopurine, 6-thioguanine, fludarabine phosphate, pentostatine, and gemcitabine and hydroxyurea); antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); proteasome inhibitors, for example carfilzomib and bortezomib; interferon therapy; and topoisomerase inhibitors (for example epipodophyllotoxins like etoposide and teniposide, amsacrine, topotecan, mitoxantrone and camptothecin); bleomcin, dactinomycin, daunorubicin, doxorubicin, epirubicin, idarubicin, ara-C, paclitaxel (Taxol™), nabpaclitaxel, docetaxel, mithramycin, deoxyco-formycin, mitomycin-C, L-asparaginase, interferons (especially IFN-a), etoposide, and teniposide;
(ii) cytostatic agents such as antiestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5*-reductase such as finasteride; and navelbene, CPT-II, anastrazole, letrazole, capecitabine, reloxafme, cyclophosphamide, ifosamide, and droloxafine;
(iii) anti-invasion agents, for example dasatinib and bosutinib (SKI-606), and metalloproteinase inhibitors, inhibitors of urokinase plasminogen activator receptor function or antibodies to Heparanase;
(iv) inhibitors of growth factor function: for example such inhibitors include growth factor antibodies and growth factor receptor antibodies, for example the anti erbB2 antibody trastuzumab [Herceptin™], the anti-EGFR antibody panitumumab, the anti erbB1 antibody cetuximab, tyrosine kinase inhibitors, for example inhibitors of the epidermal growth factor family (for example EGFR family tyrosine kinase inhibitors such as gefitinib, erlotinib, 6-acrylamido-N-(3-chloro-4-fluorophenyl)-7-(3-morpholinopropoxy)-quinazolin-4-amine (CI 1033), erbB2 tyrosine kinase inhibitors such as lapatinib) and antibodies to costimulatory molecules such as CTLA-4, 4-IBB and PD-I, or antibodies to cytokines (IL-10, TGF-beta); inhibitors of the hepatocyte growth factor family; inhibitors of the insulin growth factor family; modulators of protein regulators of cell apoptosis (for example Bcl-2 inhibitors); inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib, tipifarnib and lonafarnib), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor, kinase inhibitors; aurora kinase inhibitors and cyclin dependent kinase inhibitors such as CDK2 and/or CDK4 inhibitors; and CCR2, CCR4 or CCR6 modulator;
(v) antiangiogenic agents such as those which inhibit the effects of vascular endothelial growth factor, for example the anti vascular endothelial cell growth factor antibody bevacizumab (Avastin™); thalidomide; lenalidomide; and for example, a VEGF receptor tyrosine kinase inhibitor such as vandetanib, vatalanib, sunitinib, axitinib and pazopanib;
(vi) gene therapy approaches, including for example approaches to replace aberrant genes such as aberrant p53 or aberrant BRCA1 or BRCA2;
(vii) immunotherapy approaches, including for example antibody therapy such as alemtuzumab, rituximab, ibritumomab tiuxetan (Zevalin®) and ofatumumab; interferons such as interferon α; interleukins such as IL-2 (aldesleukin); interleukin inhibitors for example IRAK4 inhibitors; cancer vaccines including prophylactic and treatment vaccines such as HPV vaccines, for example Gardasil, Cervarix, Oncophage and Sipuleucel-T (Provenge); gp100; dendritic cell-based vaccines (such as Ad.p53 DC); and toll-like receptor modulators for example TLR-7 or TLR-9 agonists; and
(viii) cytotoxic agents for example fludaribine (fludara), cladribine, pentostatin (Nipent™);
(ix) steroids such as corticosteroids, including glucocorticoids and mineralocorticoids, for example aclometasone, aclometasone dipropionate, aldosterone, amcinonide, beclomethasone, beclomethasone dipropionate, betamethasone, betamethasone dipropionate, betamethasone sodium phosphate, betamethasone valerate, budesonide, clobetasone, clobetasone butyrate, clobetasol propionate, cloprednol, cortisone, cortisone acetate, cortivazol, deoxycortone, desonide, desoximetasone, dexamethasone, dexamethasone sodium phosphate, dexamethasone isonicotinate, difluorocortolone, fluclorolone, flumethasone, flunisolide, fluocinolone, fluocinolone acetonide, fluocinonide, fluocortin butyl, fluorocortisone, fluorocortolone, fluocortolone caproate, fluocortolone pivalate, fluorometholone, fluprednidene, fluprednidene acetate, flurandrenolone, fluticasone, fluticasone propionate, halcinonide, hydrocortisone, hydrocortisone acetate, hydrocortisone butyrate, hydrocortisone aceponate, hydrocortisone buteprate, hydrocortisone valerate, icomethasone, icomethasone enbutate, meprednisone, methylprednisolone, mometasone paramethasone, mometasone furoate monohydrate, prednicarbate, prednisolone, prednisone, tixocortol, tixocortol pivalate, triamcinolone, triamcinolone acetonide, triamcinolone alcohol and their respective pharmaceutically acceptable derivatives. A combination of steroids may be used, for example a combination of two or more steroids mentioned in this paragraph;
(x) targeted therapies, for example PI3Kd inhibitors, for example idelalisib and perifosine; PD-1, PD-L1, PD-L2 and CTL4-A modulators, antibodies and vaccines; other IDO inhibitors (such as indoximod); anti-PD-1 monoclonal antibodies (such as MK-3475 and nivolumab); anti-PD-L1 monoclonal antibodies (such as MEDI-4736 and RG-7446); anti-PD-L2 monoclonal antibodies; and anti-CTLA-4 antibodies (such as ipilimumab);
(xii) chimeric antigen receptors, anticancer vaccines and arginase inhibitors.

Such combination treatment may be achieved by way of the simultaneous, sequential or separate dosing of the individual components of the treatment. Such combination products employ the compounds of this invention within a therapeutically effective dosage range described hereinbefore and the other pharmaceutically-active agent within its approved dosage range.

Compounds of the invention may exist in a single crystal form or in a mixture of crystal forms or they may be amorphous. Thus, compounds of the invention intended for pharmaceutical use may be administered as crystalline or amorphous products. They may be obtained, for example, as solid plugs, powders, or films by methods such as precipitation, crystallization, freeze drying, or spray drying, or evaporative drying. Microwave or radio frequency drying may be used for this purpose.

For the above-mentioned compounds of the invention the dosage administered will, of course, vary with the compound employed, the mode of administration, the treatment desired and the disorder indicated. For example, if the compound of the invention is administered orally, then the daily dosage of the compound of the invention may be in the range from 0.01 micrograms per kilogram body weight (μg/kg) to 100 milligrams per kilogram body weight (mg/kg).

A compound of the invention, or pharmaceutically acceptable salt thereof, may be used on their own but will generally be administered in the form of a pharmaceutical composition in which the compounds of the invention, or pharmaceutically acceptable salt thereof, is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration of the compounds of the invention, the pharmaceutical composition which is used to administer the compounds of the invention will preferably comprise from 0.05 to 99% w (percent by weight) compounds of the invention, more preferably from 0.05 to 80% w compounds of the invention, still more preferably from 0.10 to 70% w compounds of the invention, and even more preferably from 0.10 to 50% w compounds of the invention, all percentages by weight being based on total composition.

The pharmaceutical compositions may be administered topically (e.g. to the skin) in the form, e.g., of creams, gels, lotions, solutions, suspensions, or systemically, e.g. by oral administration in the form of tablets, capsules, syrups, powders or granules; or by parenteral administration in the form of a sterile solution, suspension or emulsion for injection (including intravenous, subcutaneous, intramuscular, intravascular or infusion); by rectal administration in the form of suppositories; or by inhalation in the form of an aerosol.

For oral administration the compounds of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compounds of the invention may be admixed with, for example, a vegetable oil or polyethylene glycol. Hard gelatine capsules may contain granules of the compound using either the above-mentioned excipients for tablets. Also, liquid or semisolid formulations of the compound of the invention may be filled into hard gelatine capsules. Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, sweetening agents (such as saccharine), preservative agents and/or carboxymethylcellulose as a thickening agent or other excipients known to those skilled in art.

For intravenous (parenteral) administration the compounds of the invention may be administered as a sterile aqueous or oily solution.

The size of the dose for therapeutic purposes of compounds of the invention will naturally vary according to the nature and severity of the conditions, the age and sex of the animal or patient and the route of administration, according to well-known principles of medicine.

Dosage levels, dose frequency, and treatment durations of compounds of the invention are expected to differ depending on the formulation and clinical indication, age, and co-morbid medical conditions of the patient.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### Experimental

Solvents, reagents and starting materials were purchased from commercial vendors and used as received unless otherwise described. Intermediates for which synthesis is not described were prepared following procedures reported in publicly accessible scientific literature. All reactions were performed at room temperature unless otherwise stated. Compound identity and purity confirmations were performed by LCMS UV using a Waters Acquity SQ Detector 2 (ACQ-SQD2 #LCA081). The diode array detector wavelength was 254 nM and the MS was in positive and negative electrospray mode (m/z: 150-800). A 2 μL aliquot was injected onto a guard column (0.2 μm×2 mm filters) and UPLC column (C18, 50×2.1 mm, <2 μm) in sequence maintained at 40° C. The samples were eluted at a flow rate of 0.6 mL/min with a mobile phase system composed of A (0.1% (v/v) Formic Acid in Water) and B (0.1% (v/v) Formic Acid in Acetonitrile) according to the gradients outlined in Table 1 below. Retention times RT are reported in minutes. The following methods were also used on occasions when described throughout the experimental section, gradients are detailed in Table 1. Method 3 utilised a Shimadzu 2020 series spectrometer equipped with a binary pump and diode array detector (acquisition wavelength 214 and 254 nm) and the MS was in positive and negative electrospray mode (m/z: 100-900). 2 μL Aliquot were injected onto an Agilent Poroshell 120 EC-C18 column (2.7 μm, 4.6×50 mm) maintained at 35° C. and eluted at 1.0 ml/min using mobile phase consisting of: A: 0.05% Formic acid in water (v/v), B: 0.05% Formic acid in MeCN (v/v). Method 4 utilised an Agilent Technologies 1290 series spectrometer equipped with a binary pump and diode array detector (acquisition wavelength 214 and 254 nm) and the MS was in positive electrospray mode (m/z: 70-1000). 2 μL aliquots were injected onto an Agilent Eclipse Plus RRHD C18, (1.8 μm, 3.0×50 mm) column maintained at 40° C. and eluted at 0.8 ml/min using mobile phase consisting of: A: 0.05% Formic acid in water (v/v), B: 0.05% Formic acid in MeCN (v/v). Methods 5 and 6 utilised a Waters Acquity H-Class QDA Detector (with PDA). The diode array detector wavelength was 254 nM and the MS was in positive and negative electrospray mode (m/z: 150-800). A 2 μL aliquot was injected onto a guard column (0.2 μm×2 mm filters) and UPLC column (C18, 50×2.1 mm, <2 μm) in sequence maintained at 40° C. The samples were eluted at a flow rate of 0.6 mL/min with a mobile phase system composed of A (0.1% (v/v) Formic Acid in Water) and B (0.1% (v/v) Formic Acid in MeCN) according to the gradients outlined in Table 1 below. Retention times RT are reported in minutes.

TABLE 1

| | | |
|---|---|---|
| Time (min) | % A | % B |

Method 1 (Long acidic)

| | | |
|---|---|---|
| 0 | 95 | 5 |
| 1.1 | 95 | 5 |
| 6.1 | 5 | 95 |
| 7 | 5 | 95 |
| 7.5 | 95 | 5 |
| 8 | 95 | 5 |

Method 2 (Short acidic)

| | | |
|---|---|---|
| 0 | 95 | 5 |
| 0.3 | 95 | 5 |
| 2 | 5 | 95 |
| 2.6 | 95 | 5 |
| 3 | 95 | 5 |

Method 3

| | | |
|---|---|---|
| 0.01 | 85 | 15 |
| 1.0 | 85 | 15 |
| 4.0 | 0 | 100 |
| 4.5 | 0 | 100 |
| 4.51 | 85 | 15 |
| 5.0 | 85 | 15 |

Method 4

| | | |
|---|---|---|
| 0.00 | 80 | 20 |
| 2.65 | 20 | 80 |
| 3.00 | 20 | 80 |

Method 5 (Short acidic)

| | | |
|---|---|---|
| 0 | 95 | 5 |
| 0.3 | 95 | 5 |
| 2 | 5 | 95 |
| 2.6 | 95 | 5 |
| 3 | 95 | 5 |

Method 6 (Long acidic)

| | | |
|---|---|---|
| 0 | 95 | 5 |
| 1.1 | 95 | 5 |
| 6.1 | 5 | 95 |
| 7 | 5 | 95 |
| 7.5 | 95 | 5 |
| 8 | 95 | 5 |

NMR was also used to characterise final compounds. NMR spectra were obtained on a Bruker AVIII 400 Nanobay with 5 mm BBFO probe. Optionally, compound Rf values on silica thin layer chromatography (TLC) plates were measured. Compound purification was performed by flash column chromatography on silica or by preparative LCMS. LCMS purification was performed using a Waters 3100 Mass detector in positive and negative electrospray mode (m/z: 150-800) with a Waters 2489 UV/Vis detector. Samples were eluted at a flow rate of 20 mL/min on a XBridge™ prep C18 5 μM OBD 19×100 mm column with a mobile phase system composed of A (0.1% (v/v) Formic Acid in Water) and B (0.1% (v/v) Formic Acid in Acetonitrile) according to the gradient outlined in Table 2 below.

TABLE 2

| Time (min) | % A | % B |
|---|---|---|
| 0 | 90 | 10 |
| 1.5 | 90 | 10 |
| 11.7 | 5 | 95 |
| 13.7 | 5 | 95 |
| 14 | 90 | 90 |
| 15 | 90 | 90 |

### General Schemes

Compounds of formula (Ia) can be prepared using the steps described in general scheme 1, where LG¹, LG² and LG³ are suitable leaving groups such as chloride (Cl), bromide (Br) or iodide (I) or alkyl sulfonyl groups eg SO₂Et, or a triflate (OTf).

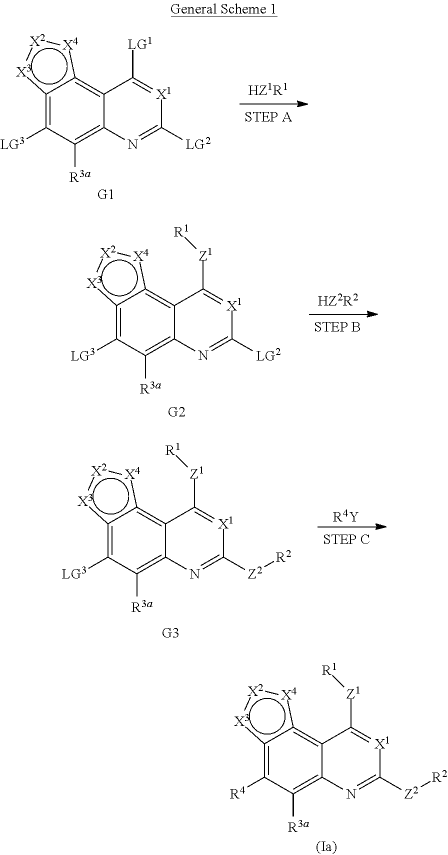

In step A G1 in which LG1 is a halogen, for example a chloride undergoes a nucleophilic substitution reaction with an alcolhol or amine (HZ1R¹) in the presence of a base, for example DIPEA in a solvent such as THF at room temperature to form G2. In step B, a second nucleophilic substitution of LG2 (where LG2 may be a chloride or on SO₂Et group) with an alcohol or amine (HZ2R²) can be effected using a solvent such as anhydrous THF and a base, for example NaH. In step C, G3 can undergo a transition metal catalysed cross coupling reaction, for example a Suzuki reaction (LG3 may be a bromide and R⁴Y a boronic acid or ester of R⁴) in the presence of an appropriate catalyst, for example [1,1′-Bis(di-tert-butylphosphino)ferrocene]dichloropalladium(II), a base such as cesium carbonate in a solvent system such as 1,4-dioxane and water at elevated temperature. Additional standard deprotection steps may be required depending on the nature of the R¹, R² and R⁴ substituents installed during steps A to C. Additionally the order of the steps can be changed so that R⁴ can be installed prior to Z¹R¹ and Z²R².

Intermediates of formula G1 can be prepared by the routes in general schemes 2 to 5. The starting compounds for each of these routes (described by formula G4) are commercially available or can be prepared by methods well described in the chemical literature. Intermediates of formula G1a (where X¹═N, LG¹=LG²=Cl and LG³=Br) can be formed according to general scheme 2 or general scheme 3.

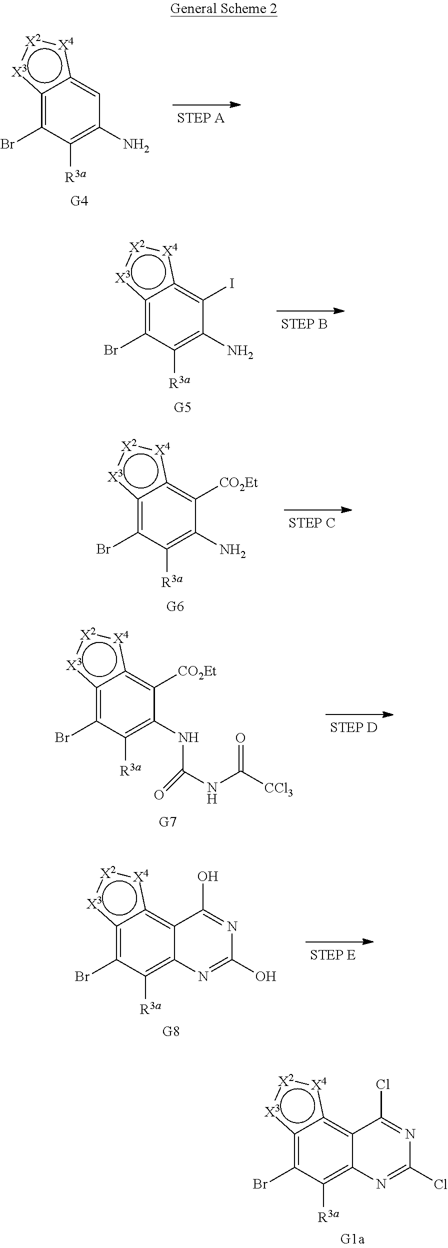

In step A aniline G4 can be ortho-iodinated using for example, NIS in MeCN at 0° C. In step B the iodide of G5 can undergo carbonylation using a carbonyl donor such as molybdenum hexacarbonyl, a catalyst such as Pd(dppf)Cl₂ and DBU in EtOH at 70° C. In step C the anilinic nitrogen of G6 is alkylated with trichloroacetyl isocyanate to afford G7 which in step D is treated with ammonia in MeOH at elevated temperature to cyclise and form dihydroxypyrimidine of formula G8 which can be chlorinated, for example using POCl₃ and DIPEA at reflux.

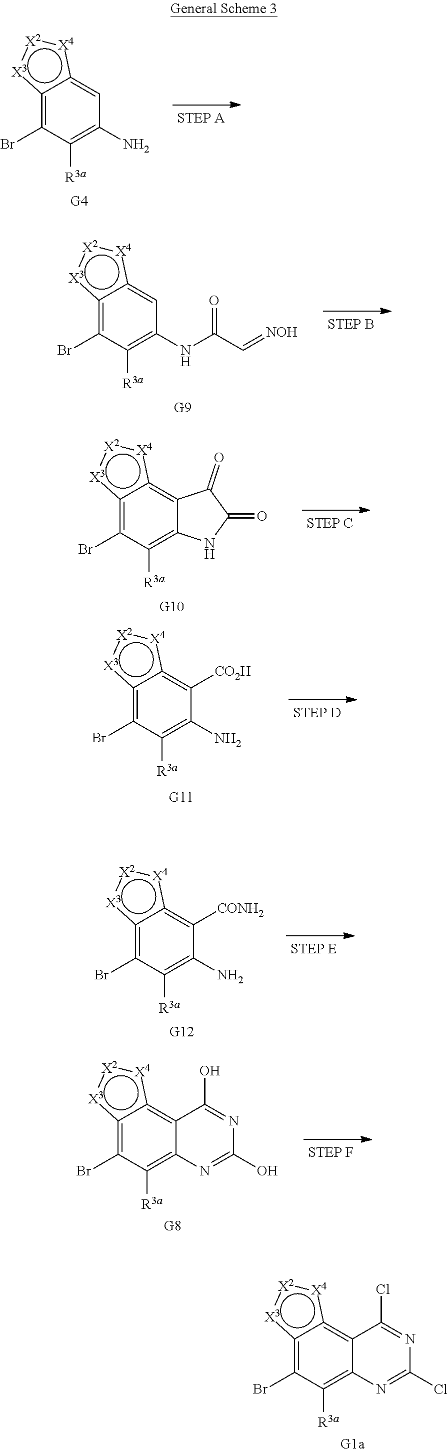

Alternatively, aniline G4 can undergo Sandmeyer isatin formation under standard conditions in steps A and B to form G10 which can be hydrolysed to a carboxylic acid of formula G11 for instance, using sodium hydroxide and sodium peroxide at room temperature in step C. Step D involves conversion of the carboxylic acid to a primary amide using an ammonia source such as ammonium chloride, a base such as DIPEA a carboxylate activating agent such as HATU and a solvent, for instance, DMF. In step E a primary amide of formula G12 is converted to a dihydroxypyrimidine of formula G8 with a carbonyl donor, for example, CDI in a solvent such as THF at reflux. Finally chlorination of dihydroxpyrimidines of formula G8 affords compounds of formula G1a, this can be done using POCl₃ as previously described in general scheme 2.

Intermediates of formula G1b (where X¹═N, LG¹=Cl, LG²=SO₂Et and LG³=Br) can be formed according to the route described in general scheme 4.

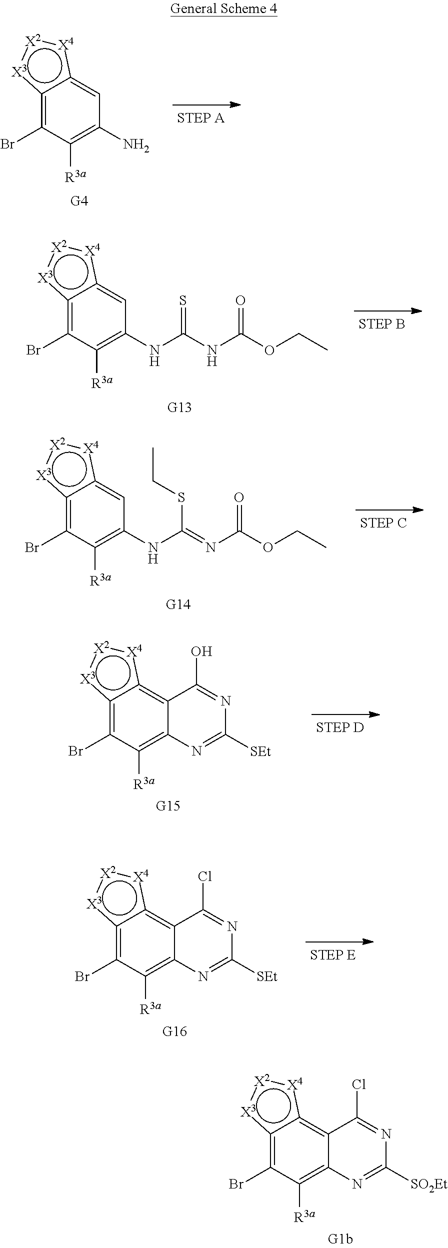

In step A aniline G4 can be reacted with a isothiocyanate such as ethoxycarbonyl isothiocyanate in DCM or another suitably inert solvent to form a thiourea of formula G13. G13 can be S-alkylated to form G14 in step B using an alkylating agent such as ethyl iodide in DMF with a base such as potassium carbonate present. Cyclisation to G15 can be effected in step C by heating at elevated temperature in a high boiling solvent, this may be done using microwave irradiation to achieve a temperature of 170° C. in NMP as solvent. Step D involves chlorination of the hydroxy group of G15, for example this can be done with POCl₃ in DMF at 90° C. Finally, in step E, G16 can be converted to an intermediate of formula G1b by oxidation of the thioether linkage under suitable conditions, for instance, using a peracid such as mCPBA in DCM at room temperature.
Intermediates of structure G1c (where X¹═C, LG¹=LG²=Cl and LG³=Br) can be formed according to the route described in general scheme 5, treating an aniline of formula G4 with malonic acid in POCl₃ at reflux.

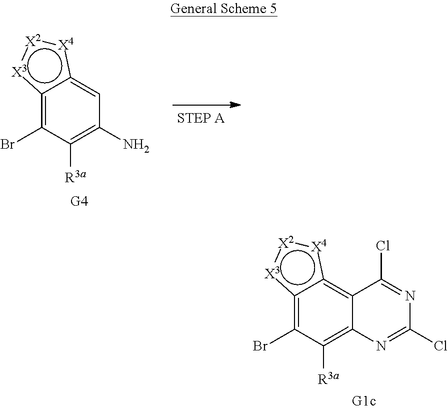

### Synthesis of Intermediates

### Intermediate A-1, 2-[3-(methoxymethoxy)-1-naphthyl]-4,4,5,5-tetramethyl-1,3,2-dioxaborolane

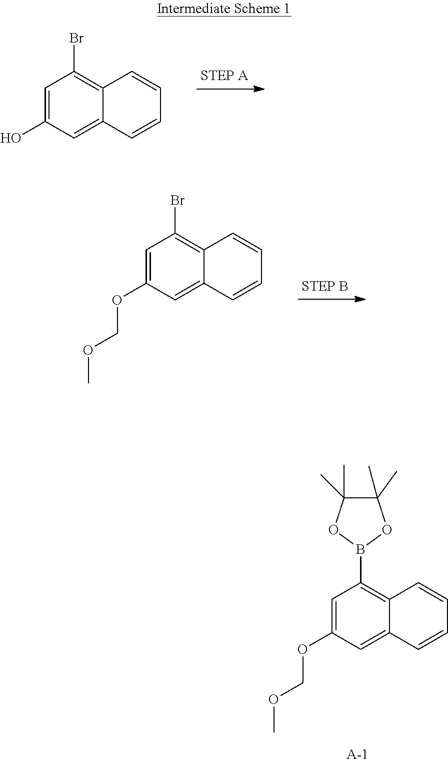 [SMILES: Oc1cc(Br)c2ccccc2c1 ; COCOc1cc(Br)c2ccccc2c1 ; COCOc1cc(B2OC(C)(C)C(C)(C)O2)c2ccccc2c1 ; "A-1"]

Step A, 3-(methoxymethoxy) naphthalene. To a suspension of 4-bromonaphthalen-2-ol (3 g, 13.45 mmol) and N,N-diisopropylethylamine (7.03 mL, 40.35 mmol) in DCM (30 mL) at 0° C. was added chloromethyl methyl ether (1.53 mL, 20.17 mmol). The resulting mixture was stirred for 30 min. The reaction was then diluted with distilled water, extracted with DCM (×2), organics were combined and washed with brine (×2), dried over Na₂SO₄, filtered and the filtrate evaporated in vacuo to afford a reddish/pink oil. This was purified by flash column chromatography eluting 0-60% EtOAc in pet. ether (40 g SiO₂, dry loaded with DCM). The desired fractions were combined and evaporated in vacuo to afford a pink oil analysed as 1-bromo-3-(methoxymethoxy) naphthalene (3.2 g, 11.9 mmol, 89% yield).

UPLC-MS (ES⁺, Method 1): 2.06 min, m/z 266.9, 268.9 [M+H]⁺

¹H NMR (400 MHz, CDCl₃) δ/ppm: 8.19-8.15 (1H, m), 7.77-7.73 (1H, m), 7.60 (1H, d, J=2.4 Hz), 7.53-7.46 (2H, m), 7.42-7.40 (1H, m), 5.31 (2H, s), 3.55 (3H, s).

Step B, 2-[3-(methoxymethoxy)-1-naphthyl]-4,4,5,5-tetramethyl-1,3,2-dioxaborolane (A-1). To a nitrogen purged suspension of 1-bromo-3-(methoxymethoxy) naphthalene (5.2 g, 19.4 mmol), bis(pinacolato)diboron (9.9 g, 38.9 mmol) and potassium acetate (6.7 g, 68 mmol) in toluene (50 mL) was added [1,1′bis(diphenylphosphino)ferrocene]dichloropalladium(II) (1.4 g, 1.9 mmol). The mixture was stirred at 110° C. for 2.5 hrs. The reaction mixture was then filtered over celite, washing with EtOAc and the filtrate evaporated in vacuo to afford a black oil. This was taken up in distilled water and EtOAc, extracted aqueous (×2) with EtOAc, combined organics were washed with brine, dried over Na₂SO₄, filtered and evaporated in vacuo to afford a black oil. This was purified by flash column chromatography (40 g SiO₂, dry loaded with DCM) eluting with 0-20% EtOAc in petrol ether, desired fractions were combined and evaporated in vacuo to afford waxy colourless residue. Analysed as 2-[3-(methoxymethoxy)-1-naphthyl]-4,4,5,5-tetramethyl-1,3,2-dioxaborolane (6 g, 98% yield).

UPLC-MS (ES⁺, Method 2): 2.16 min, m/z 315 [M+H]⁺

¹H NMR (400 MHz, D₆-DMSO) δ/ppm: 8.56-8.59 (1H, d, J=7.1 Hz), 7.86-7.89 (1H, d, J=7.1 Hz), 7.63-7.65 (1H, d, J=2.4 Hz), 7.58-7.60 (1H, d, J=2.4 Hz), 7.40-7.50 (2H, m), 5.31 (2H, s), 3.55 (3H, s), 1.2 (s, 12H).

### Intermediate A-2, 1,2,3,5,6,7-hexahydropyrrolizin-8-ylmethanol (A-2)

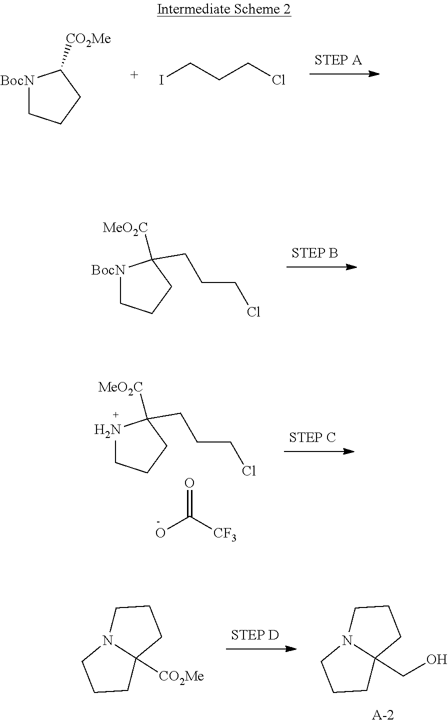

Step A, O1-tert-butyl O-2-methyl 2-(3-chloropropyl) pyrrolidine-1,2-dicarboxylate. A 1M solution of lithium bis(trimethylsilyl)amide (43.6 mL, 43.6 mmol) in THF was added to a solution of N-boc-Proline methyl ester (10 g, 43.6 mmol) in THF (100 mL) at −78° C. Afterwards, the mixture allowed to stir at that temperature for 30 min. 3-chloropropyl iodide (5.6 mL, 52.3 mmol) was added and reaction mixture was allowed to gradually warm up to 0° C. After 2 hrs in ice bath TLC (3:1 pet. ether:EtOAc) shows complete consumption of starting material (vis. with |2). Reaction mixture was quenched with a saturated solution of aqueous ammonium chloride (50 mL), partitioned between a layer of ethyl acetate (100 mL) and water (50 mL). The organic layer was separated. The aqueous layer was extracted with ethyl acetate (100 mL), organic extracts were combined, washed with a saturated solution of brine (100 mL), dried over sodium sulfate, filtered and concentrated under reduced pressure to afford a pale yellow oil analysed as O1-tert-butyl O2-methyl 2-(3-chloropropyl) pyrrolidine-1,2-dicarboxylate (13 g, 42.5 mmol, 97% yield).

¹H NMR (400 MHz, CDCl₃) δ/ppm: 3.50-3.62 (4H, m), 3.35-3.45 (2H, m), 3.19-3.23 (1H, m), 2.11-2.20 m, 1H), 1.50-2.05 (m, 6H), 1.25-1.29 (9H, d).

Step B, Methyl 2-(3-chloropropyl) pyrrolidin-1-ium-2-carboxylate; 2,2,2-trifluoroacetate. Trifluoroacetic acid (10 mL, 130.2 mmol) was added to a solution of O1-tert-butyl O2-methyl 2-(3-chloropropyl) pyrrolidine-1,2-dicarboxylate (13 g, 42.5 mmol) in DCM (22 mL). The resulting solution was allowed to stir at room temp overnight. All volatiles removed under reduced pressure and dark oil re-dissolved in DCM and evaporated again to remove TFA. The resulting dark oil was analysed as methyl 2-(3-chloropropyl) pyrrolidin-1-ium-2-carboxylate; 2,2,2-trifluoroacetate (19 g, 59.4 mmol, 140% yield). NMR indicates product is TFA salt and contains approximately 1 equiv. of TFA remains, used without further purification.

¹H NMR (400 MHz, CDCl₃)/ppm: 10.20-10.45 (1H, bs), 7.70-8-02 (1H, bs), 3.91 (s, 3H), 3.45-3.70 (m, 4H), 2.45-2.55 (1H, m), 2.26-2.31 (1H, m), 2.15-2.25 (3H, m), 1.95-2.04 (2H, m), 1.55-1.65 (1H, m).

Step C, Methyl 1,2,3,5,6,7-hexahydropyrrolizine-8-carboxylate. Potassium carbonate (27 g, 195.4 mmol) was added to a mixture of potassium iodide (1 g, 6.02 mmol) and methyl 2-(3-chloropropyl) pyrrolidin-1-ium-2-carboxylate; 2,2,2-trifluoroacetate (13.5 g, 42.2 mmol) in methanol (200 mL). The mixture was allowed to stir at 35° C. for 90 mins before concentrating under reduced pressure. Then the reaction mixture was partitioned between a layer of DCM (150 mL) and water (150 mL). The organic layer was separated. The aqueous layer was extracted with DCM (100 mL). The organic extracts were combined, washed with brine (100 ml) dried (Na₂SO₄), filtered and concentrated under reduced pressure to afford methyl 1,2,3,5,6,7-hexahydropyrrolizine-8-carboxylate (6.5 g, 38.4 mmol, 91% yield) as a clear oil.

¹H NMR (400 MHz, CDCl₃) δ/ppm: 3.73 (3H, s), 3.12-3.20 (2H, m), 2.62-2.70 (2H, m), 2.25-2.31 (2H, m), 1.78-1.85 (4H, m), 1.62-1.74 (2H, m).

Step D, 1,2,3,5,6,7-hexahydropyrrolizin-8-ylmethanol (A-2). At 0° C., under an N₂ atmosphere a 1M solution of lithium aluminium hydride (19.5 mL, 19.5 mmol) in THF was added dropwise to a solution of methyl 1,2,3,5,6,7-hexahydropyrrolizine-8-carboxylate (1.1 g, 6.5 mmol) in THF (10 mL). The mixture was allowed to stir at that temperature for 30 mins. Maintaining temp of 0° C., inert atmosphere and using vigorous stirring, the reaction was quenched with dropwise addition of water (0.7 mL) then dropwise addition of a 15% aq. NaOH (0.7 mL) followed by more water (2 ml). The mixture was allowed to stir and warm up to room temperature until the precipitated salts dispersed into a freely moving suspension, before filtering, washing the filter cake with THF (2×10 mL). The filtrate was collected and concentrated under reduced pressure to afford 1,2,3,5,6,7-hexahydropyrrolizin-8-ylmethanol (1.0 g, 7.1 mmol, 100% yield) as a yellow oil.

¹H NMR (400 MHz, CDCl₃) δ/ppm: 3.75-3.79 (1H, m), 3.30 (2H, s), 2.98-3.03 (2H, m), 2.59-2.65 (2H, m), 1.51-1.92 (8H, m).

### Intermediate A-3, [(2R,8S)-2-Fluoro-1,2,3,5,6,7-hexahydropyrrolizin-8-yl]methanol

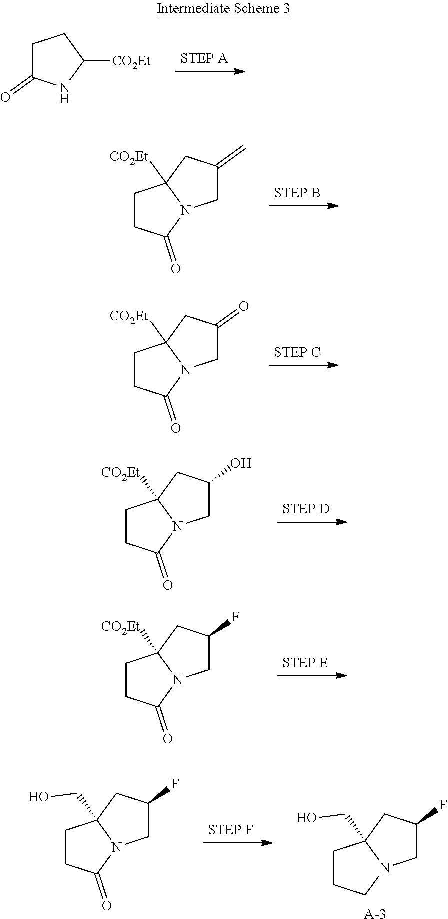

Step A, Ethyl 6-methylene-3-oxo-1,2,5,7-tetrahydropyrrolizine-8-carboxylate. At −40° C., LIHMDS (1.34 L, 1.34 mol, 2.1 eq) was added dropwise into a solution of ethyl 5-oxo-2-pyrrolidinecarboxylate (100 g, 637 mmol, 1.0 eq) and 3-chloro-2-chloromethyl-1-propene (239 g, 1.91 mol, 3.0 eq) in THF (2000 mL). The mixture was allowed to warm to room temperature and stir over night. The mixture was cooled to −60° C. and adjusted to pH 7 with 2M HCl, poured into water (10 L), extracted with EtOAc (2 L×3), washed with brine (5 L) and dried over Na₂SO₄. The filtered mixture was concentrated and purified by silica gel column (5:1 Pet.ether/EtOAc to 3:1 Pet.ether/EtOAc) to afford ethyl 6-methylene-3-oxo-1,2,5,7-tetrahydropyrrolizine-8-carboxylate as a colourless oil (40 g, 30% yield).

¹H NMR (400 MHz, CDCl₃) δ/ppm: 5.01-5.06 (2H, d), 4.16-4.30 (3H, m), 3.69-3.73 (1H, d), 3.02-3.06 (1H, d), 2.30-2.75 (4H, m), 2.07-2.16 (1H, m), 1.23-1.27 (3H, t).

Step B, Ethyl 3,6-dioxo-1,2,5,7-tetrahydropyrrolizine-8-carboxylate. At −78° C., O2 was bubbled through a solution of ethyl 6-methylene-3-oxo-1,2,5,7-tetrahydropyrrolizine-8-carboxylate (115.1 g, 550 mmol, 1.0 eq) in DCM (1 L) and MeOH (100 mL) for 30 minutes. Ozone was then bubbled through the solution with stirring at −78° C. until the solution became blue. O2 was then bubbled through the solution at the same temperature for a further 30 min. Dimethyl sulfide (68.35 g, 1.1 mol, 2.0 eq) was added at −78° C. and the solution was allowed to reach room temperature and stir over night. The mixture was concentrated and purified by silica gel column (5:1 Pet.ether/EtOAc to 3:1 Pet.ether/EtOAc) to give ethyl 3,6-dioxo-1,2,5,7-tetrahydropyrrolizine-8-carboxylate (108 g, 93% yield) as a colourless oil.

¹H NMR (400 MHz, CDCl₃) δ/ppm: 4.20-4.24 (2H, q), 4.08-4.14 (1H, d), 3.52-3.56 (1H, d), 2.95-3.00 (2H, m), 2.81-2.86 (1H, m), 2.39-2.48 (2H, m), 2.19-2.24 (1H, m), 1.26-1.29 (3H, t).

Step C Ethyl(2S,8S)-2-hydroxy-5-oxo-2,3,6,7-tetrahydro-1H-pyrrolizine-8-carboxylate. At 0° C., NaBH₄ (5.81 g, 153 mmol, 0.3 eq) was added to a solution of ethyl 3,6-dioxo-1,2,5,7-tetrahydropyrrolizine-8-carboxylate (108 g, 512 mmol, 1.0 eq) in EtOH (550 mL) and stirred for 10 min. To the mixture was added aqueous NH₄Cl (50 mL) before stirring for a further 20 min at 0° C. The mixture was concentrated in vacuo and the crude was purified by silica gel column (30:1 DCM/MeOH) to afford ethyl(2S,8S)-2-hydroxy-5-oxo-2,3,6,7-tetrahydro-1H-pyrrolizine-8-carboxylate as a yellow oil and apparent mixture of sterioisomers (88 g, 81% yield).

LC-MS (ES⁺, Method 4): 0.40 min, 214.10 [M+H]⁺.

Step D, Ethyl(2R,8S)-2-fluoro-5-oxo-2,3,6,7-tetrahydro-1H-pyrrolizine-8-carboxylate. At −78° C., DAST (99.8 g, 619 mmol, 1.5 eq) was added dropwise into a solution of rac-ethyl(2S,8S)-2-hydroxy-5-oxo-2,3,6,7-tetrahydro-1H-pyrrolizine-8-carboxylate (88 g, 413 mmol, 1.0 eq) in DCM (1.5 L) and stirred at room temperature overnight. The mixture was cooled to 0° C. before adding MeOH (60 mL) and diluting with brine (2000 mL). The phases were separated and the organics dried over Na₂SO₄, filtered and concentrated under reduced pressure. The crude was purified by silica gel column (8:1 to 5:1 pet. Ether/EtOAc) to give ethyl(2R,8S)-2-fluoro-5-oxo-2,3,6,7-tetrahydro-1H-pyrrolizine-8-carboxylate as a yellow oil (42 g, 47% yield).

¹H NMR (400 MHz, CDCl₃) δ/ppm: 5.21-5.36 (1H, d), 4.20-4.47 (3H, m), 3.19-3.41 (1H, dd), 2.10-2.60 (6H, m), 1.24-1.28 (3H, t).

Step E, (6R,8S)-6-Fluoro-8-(hydroxymethyl)-2,5,6,7-tetrahydro-1H-pyrrolizin-3-one. At 0° C., LiBH₄ (81 mL, 163 mmol, 1.0 eq) was added dropwise into ethyl(2R,8S)-2-fluoro-5-oxo-2,3,6,7-tetrahydro-1H-pyrrolizine-8-carboxylate (35 g, 163 mmol, 1.0 eq) in THF (350 mL) and stirred at room temperature for 2 hrs. The mixture was cooled to 0° C., before adding aqueous NH₄Cl (100 mL) and stirring for 30 mins at 0° C., the mixture was concentrated and the crude was purified by silica gel column (30:1 DCM/MeOH) to give (6R,8S)-6-fluoro-8-(hydroxymethyl)-2,5,6,7-tetrahydro-1H-pyrrolizin-3-one (28 g, 99% yield).

¹H NMR (400 MHz, CDCl₃)/ppm: 5.21-5.35 (1H, d), 4.05-4.16 (1H, m), 3.51-3.55 (1H, d), 3.42-3.46 (1H, d) 2.52-3.11 (3H, m), 1.94-2.41 (5H, m).

Step F, [(2R,8S)-2-Fluoro-1,2,3,5,6,7-hexahydropyrrolizin-8-yl]methanol (A-3). At 0° C., BH₃. DMS (10M, 75.1 mL, 751 mmol, 5.0 eq) was added dropwise into rac-(6R,8S)-6-fluoro-8-(hydroxymethyl)-2,5,6,7-tetrahydro-1H-pyrrolizin-3-one (26 g, 150 mmol, 1.0 eq) in THF (1300 mL) and stirred at room temperature overnight. The mixture was cooled to 0° C., MeOH (300 mL) was added and stirring continued for 1 hr at 0° C. The mixture was concentrated and the crude was dissolved in MeOH (300 mL) and stirred at 50° C. overnight. The mixture was concentrated to give (21 g, 88% yield) of [(2R,8S)-2-fluoro-1,2,3,5,6,7-hexahydropyrrolizin-8-yl]methanol, intermediate 21 as a colourless oil.

¹H NMR (400 MHz, CDCl₃) δ/ppm: 5.11-525 (1H, d), 2.80-3.31 (6H, m), 1.74-2.11 (6H, m).

### Intermediate A-4, 6-chloro-N,N-bis[(4-methoxyphenyl)methyl]-4-methyl-5-(trifluoromethyl)pyridine-2-amine

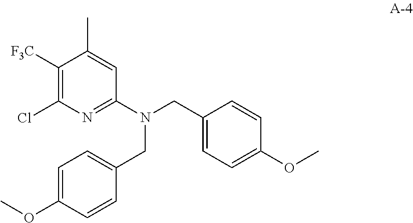

Intermediate A-4 was prepared following the route described in Intermediate Scheme 4.

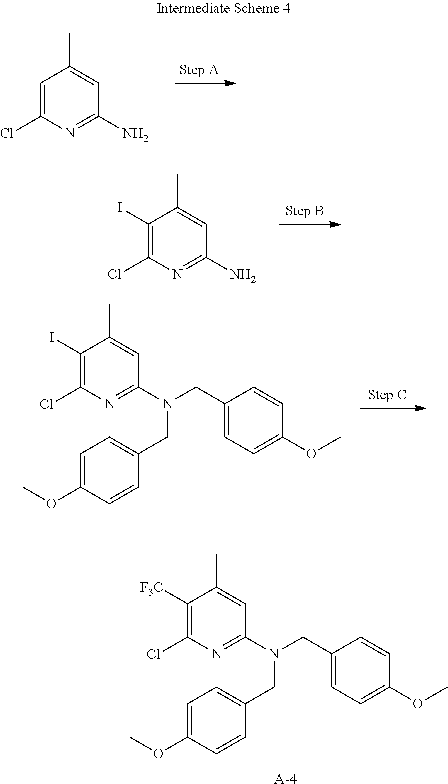

Step A, 6-chloro-5-iodo-4-methyl-pyridin-2-amine. To a solution of 6-chloro-4-methyl-2-pyridinamine (1.00 g, 7.01 mmol) in MeCN (16 mL) was added N-Iodosuccinimide (1.89 g, 8.42 mmol). The reaction mixture was stirred at rt for 2 h. The reaction mixture was concentrated to dryness and partitioned between DCM and brine. The organics were extracted, passed through phase separating filter paper and concentrated in vacuo. The crude material was purified by column chromatography eluting 10-100% EtOAc in pet ether. The desired fractions were combined and concentrated to dryness to provide 6-chloro-5-iodo-4-methyl-pyridin-2-amine (1.52 g, 5.66 mmol, 81% yield) as an orange solid.

UPLC-MS (ES⁺, Method 2): 1.76 min, m/z 268.8/270.8 [M+H]⁺

Step B, 6-chloro-5-iodo-N,N-bis[(4-methoxyphenyl)methyl]-4-methyl-pyridin-2-amine. To a solution of 6-chloro-5-iodo-4-methyl-pyridin-2-amine (1.50 g, 5.59 mmol) in DMF (20 mL) was added 4-methoxybenzylchloride (2.3 mL, 16.8 mmol) and sodium hydride, (60% dispersed in mineral oil) (536 mg, 22.4 mmol) and the reaction stirred at rt for 16 h. The reaction mixture was partitioned between EtOAc and water. The organics were separated and the aqueous layer was washed three times with EtOAc. The combined organic washings were washed with brine and passed through phase separating filter paper before concentrating to dryness. The crude material was then purified by column chromatography eluting 10% EtOAc in pet. ether to 100% EtOAc. The desired fractions were combined and concentrated in vacuo to provide 6-chloro-5-iodo-N,N-bis[(4-methoxyphenyl)methyl]-4-methyl-pyridin-2-amine (2.83 g, 5.56 mmol, 100% yield) as a yellow solid.

UPLC-MS (ES⁺, Method 2): 2.59 min, m/z 509.0 [M+H]⁺

Step C, 6-chloro-N,N-bis[(4-methoxyphenyl)methyl]-4-methyl-5-(trifluoromethyl)pyridin-2-amine (A-4). To a solution of 6-chloro-5-iodo-N,N-bis[(4-methoxyphenyl)methyl]-4-methyl-pyridin-2-amine (1.00 g, 1.97 mmol) in DMF (10 mL) was added Copper (I) iodide (749 mg, 3.93 mmol) and methyl-2,2-difluoro-2-(fluorosulfonyl)acetate (0.5 mL, 3.93 mmol) and the reaction heated to 90° C. for 16 h. The reaction was diluted with EtOAc and water, the organics extracted, passed through phase separating filter paper and concentrated to dryness. The crude material was then purified by flash column chromatography eluting 0-60% EtOAc in pet. ether. The desired fractions were combined and concentrated in vacuo to provide 6-chloro-N,N-bis[(4-methoxyphenyl)methyl]-4-methyl-5-(trifluoromethyl)pyridin-2-amine (563 mg, 1.2487 mmol, 64% yield) as an orange oil which solidified to a white solid when left standing.

UPLC-MS (ES⁺, Method 2): 2.51 min, m/z 451.1 [M+H]⁺

### Intermediate A-5, 5-(2-nitrophenyl) sulfonyl-4,6,7,8-tetrahydropyrazolo[1,5-a][1,4]diazepine-2-carboxylic acid

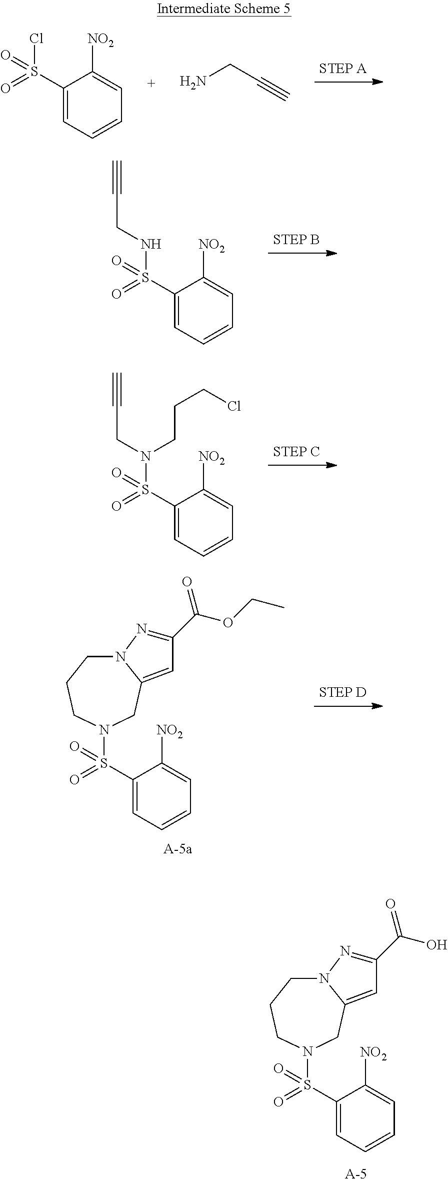

Step A, 2-nitro-N-prop-2-ynyl-benzenesulfonamide. A solution of propargylamine (37.28 g, 676.83 mmol) and N,N-diisopropylethylamine (129.81 mL, 1353.7 mmol) in DCM (1.86 L) was cooled to 0° C. 2-Nitrobenzenesulfonyl chloride (149.99 g, 676.83 mmol) was added portion-wise. The reaction mixture was then allowed to warm to room temperature and left to stir at room temperature overnight. The reaction mixture was concentrated in vacuo and the crude residue purified by flash column chromatography (SiO₂, 20-50% EtOAc/PE) to afford the desired product 2-nitro-N-prop-2-ynyl-benzenesulfonamide (151.6 g, 75% yield) as a white solid.

LC-MS (ES⁺, Method 3): 1.23 min, 241.05 [M+H]⁺

Step B, N-(3-chloropropyl)-2-nitro-N-prop-2-ynyl-benzenesulfonamide. 1-bromo-3-chloropropane (245.75 g, 1561 mmol) was added dropwise to a stirring mixture of 2-nitro-n-prop-2-ynyl-benzenesulfonamide (50 g, 208.13 mmol) and cesium carbonate (169.54 g, 520.33 mmol) in acetone (2 L). The reaction mixture was left to stir at room temperature for 5 hours. The reaction mixture was concentrated in vacuo and the crude residue partitioned between EtOAc (3 L) and water (2 L). The aqueous layer was twice extracted with additional EtOAC (1.5 L), the combined organic layers were dried with Na₂SO₄, concentrated in vacuo and purified by flash column chromatography (SiO₂, PE/EA=2/1) to afford the desired product N-(3-chloropropyl)-2-nitro-N-prop-2-ynyl-benzenesulfonamide (43.48 g, 63% yield) as yellow solid.

LC-MS (ES⁺, Method 3): 1.70 min, 317.0 [M+H]⁺

Step C, ethyl 5-(2-nitrophenyl) sulfonyl-4,6,7,8-tetrahydropyrazolo[1,5-a][1,4]diazepine-2-carboxylate. N,N-diisopropylethylamine (1.12 mL, 6.4 mmol) was added to a stirring solution of n-(3-chloropropyl)-2-nitro-n-prop-2-ynyl-benzenesulfonamide (2. g, 6.31 mmol) and ethyldiazoacetate (1.09 mL, 9.54 mmol) in benzene (6 mL). The reaction mixture was irradiated in the microwave at 140° C. for 1 hour. The reaction mixture was left to cool to room temperature before cesium carbonate (2.49 g, 7.64 mmol) and THF (2 mL) were added and the reaction mixture again irradiated 140° C. for 30 minutes. The reaction mixture was left to cool to room temperature and the solvents removed in vacuo. The crude residue was partitioned between water (180 mL) and EtOAc (80 mL). The aqueous layer was washed with additional EtOAc (150 mL) and the combined organic layers washed with brine (100 mL×2). The organic layer was dried over anhydrous Na₂SO₄, the solvents removed in vacuo and concentrated and purified by flash column chromatography (SiO₂, PE/EA=1/1) to afford the desired product ethyl 5-(2-nitrophenyl) sulfonyl-4,6,7,8-tetrahydropyrazolo[1,5-a][1,4]diazepine-2-carboxylate (1.7 g, 61% yield) as yellow oil.

LC-MS (ES⁺, Method 3): 1.47 min, 395.05 [M+H]⁺

Step D, 5-(2-nitrophenyl) sulfonyl-4,6,7,8-tetrahydropyrazolo[1,5-a][1,4]diazepine-2-carboxylic acid (A-5). A solution of ethyl 5-(2-nitrophenyl) sulfonyl-4,6,7,8-tetrahydropyrazolo[1,5-a][1,4]diazepine-2-carboxylate (1.7 g, 4.31 mmol) and 1M LiOH (17.2 mL, 17.24 mmol) in THF (40 mL) and Methanol (10 mL) was left to stir at room temperature for 2 hours. The reaction mixture was concentrated in vacuo and the residue acidified to pH 3 with slow addition of 1M HCl. The resulting solid was collected via filtration to afford the desired product 5-(2-nitrophenyl) sulfonyl-4,6,7,8-tetrahydropyrazolo[1,5-a][1,4]diazepine-2-carboxylic acid (1.5 g, 86% yield) as white solid.

LC-MS (ES⁺, Method 3): 1.10 min, 367.05 [M+H]⁺

### Intermediate A-6, N-ethyl-5,6,7,8-tetrahydro-4H-pyrazolo[1,5-a][1,4]diazepine-2-carboxamide

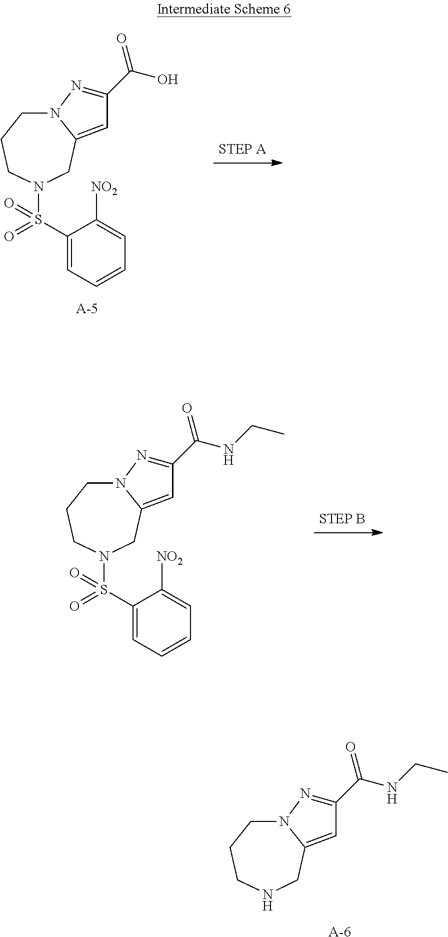

Step A, N-ethyl-5-(2-nitrophenyl) sulfonyl-4,6,7,8-tetrahydropyrazolo[1,5-a][1,4]diazepine-2-carboxamide. TBTU (1.45 g, 4.5 mmol) and N,N-diisopropylethylamine (2.65 g, 20.47 mmol) were added to a stirring mixture of 5-(2-nitrophenyl) sulfonyl-4,6,7,8-tetrahydropyrazolo[1,5-a][1,4]diazepine-2-carboxylic acid (1.5 g, 4.09 mmol) and ethanamine hydrochloride (330 mg, 4.09 mmol) at 0° C. under N₂. The reaction mixture was left to stir at 0° C. for 15 minutes before being left to warm to room temperature and stirred for 4 hours. The reaction mixture was diluted with water (600 mL) and extracted with EtOAc (200 mL×3). The combined organic layers were washed with brine (200 mL×2), dried over Na₂SO₄ and concentrated in vacuo to afford the crude product. The crude mixture was purified by flash column chromatography (SiO₂, DCM/MeOH=20/1) to afford the desired product N-ethyl-5-(2-nitrophenyl) sulfonyl-4,6,7,8-tetrahydropyrazolo[1,5-a][1,4]diazepine-2-carboxamide (1.75 g, 97.8%) as a brown oil.

LC-MS (ES+, Method 4): 1.23 min, 394.10 [M+H]⁺

Step B, N-ethyl-5,6,7,8-tetrahydro-4H-pyrazolo[1,5-a][1,4]diazepine-2-carboxamide (A-6). A mixture of N-ethyl-5-(2-nitrophenyl) sulfonyl-4,6,7,8-tetrahydropyrazolo[1,5-a][1,4]diazepine-2-carboxamide (1.75 g, 4.45 mmol), Thiophenol (0.98 g, 8.9 mmol) and cesium carbonate (2.9 g, 8.9 mmol) in MeCN (40 mL) was stirred at room temperature for 1 hour. The reaction mixture was concentrated in vacuo and the crude residue purified by flash column chromatography (SiO₂, DCM/MeOH=10/1) to afford the desired product N-ethyl-5,6,7,8-tetrahydro-4H-pyrazolo[1,5-a][1,4]diazepine-2-carboxamide (700 mg, 68% yield) as a yellow solid.

LC-MS (ES+, Method 4): 0.17 min, 209.15 [M+H]⁺

The intermediates in table 3 below were prepared by analogy with A-6 (intermediate scheme 6), replacing the amine building block ethanamine hydrochloride as described in the table entry.

TABLE 3

| Intermediate | | |
|---|---|---|
| Building block | Intermediate Structure/Name | UPLC-MS (ES⁺): min, m/z |
| Intermediate A-5 and | Intermediate A-7 | 0.26 min, 209.1 [M + H]⁺ |
| 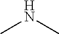 Dimethylamine hydrochloride | 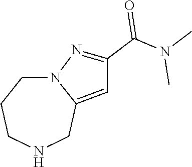 | (Method 3) |
| | A-7 | |
| | N,N-dimethyl-5,6,7,8-tetrahydro- | |
| | 4H-pyrazolo[1,5-a][1,4]diazepine- | |
| | 2-carboxamide (A-7) | |
| Intermediate A-5 and | Intermediate A-8 | 0.39 min, 235.0 [M + H]⁺ |
| 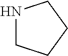 Pyrrolidine | 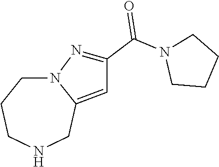 [SMILES: O=C(c1cc2n(n1)CCCNC2)N1CCCC1] | (Method 4) |
| | A-8 | |
| | pyrrolidin-1-yl(5,6,7,8-tetrahydro- | |
| | 4H-pyrazolo[1,5-a][1,4]diazepin-2- | |
| | yl)methanone (A-8) | |
| Intermediate A-5 and | Intermediate A-9 | 0.23 min, 251.1 [M + H]⁺ |
| 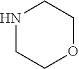 Morpholine | 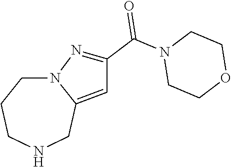 [SMILES: O=C(c1cc2n(n1)CCCNC2)N1CCOCC1] | (Method 3) |
| | A-9 | |
| | morpholino(5,6,7,8-tetrahydro-4H- | |
| | pyrazolo[1,5-a][1,4]diazepin-2- | |
| | yl)methanone (A-9) | |

### Intermediate A-10, 5,6,7,8-tetrahydro-4H-pyrazolo[1,5-a][1,4]diazepine (A-10)

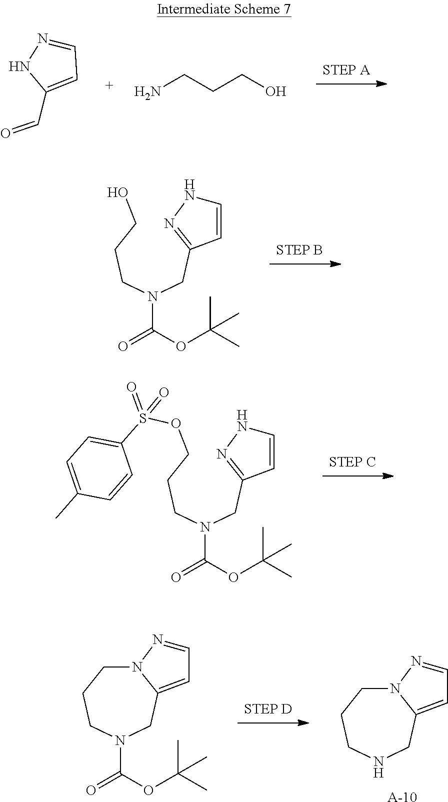

Step A, tert-butyl N-(3-hydroxypropyl)-N-(1H-pyrazol-3-ylmethyl)carbamate. A stirring suspension of 1H-pyrazole-3-carbaldehyde (1.9 g, 19.77 mmol) and 3-amino-1-propanol (2.27 mL, 29.66 mmol) in Methanol (400 mL) was cooled to 0° C. and stirred for 30 mins. Sodium Borohydride (1.5 g, 39.55 mmol) was added portionwise over 30 mins maintaining temperature <10° C. and the mixture left to stir for 1 hour. Water (15 mL) was slowly added and the mixture stirred for 40 min before portionwise addition of Di-tert-butyl dicarbonate (8.63 g, 39.55 mmol) and the reaction mixture left to stir for 72 hours. The reaction mixture was diluted with water and brine and extracted with EtOAc. Organic portions were combined and washed with brine and dried over Na₂SO₄, filtered, and concentrated to obtain the crude product as an orange oil. The crude material was purified by flash column chromatography (eluting in 0-100% EtOAc in Pet. Ether) to afford the desired product tert-butyl N-(3-hydroxypropyl)-N-(1H-pyrazol-3-ylmethyl)carbamate (1.92 g, 7.5203 mmol, 38% yield) as a pale yellow oil.

UPLC-MS (ES+, Method 2): 1.28 min, 254.2 [M−H]⁺

¹H NMR (400 MHz, DMSO-d6) δ/ppm: 12.59 (s, 1H), 7.62 (s, 1H), 6.08 (s, 1H), 4.39 (m, 1H), 4.31 (s, 2H), 3.36 (m, 2H), 3.17 (s, 2H), 1.58 (m, 2H), 1.40 (s, 9H).

Step B, 3-[tert-butoxycarbonyl(1H-pyrazol-3-ylmethyl)amino]propyl 4-methylbenzenesulfonate. A stirring solution of tert-butyl N-(3-hydroxypropyl)-N-(1H-pyrazol-3-ylmethyl)carbamate (1.92 g, 7.52 mmol) in DCM (75.203 mL) was cooled to 0° C. and stirred for 10 min before adding p-toluenesulfonyl chloride (1.72 g, 9.02 mmol) and triethylamine (1.57 mL, 11.28 mmol). The reaction mixture was left to stir for 4.5 hours. Additional p-toluenesulfonyl chloride (358.43 mg, 1.88 mmol) and triethylamine (1.57 mL, 11.28 mmol) were added and the reaction mixture was left to stir for a further hour. The reaction mixture was diluted with water (100 mL) and extracted with DCM (2×50 mL). Organic fractions were combined, dried over Na₂SO₄, filtered, and concentrated to afford a brown oil. The crude material was purified by flash column chromatography (80 g, 10-80% EtAOc in pet. ether), fractions were combined and concentrated to afford 3-[tert-butoxycarbonyl(1H-pyrazol-3-ylmethyl)amino]propyl 4-methylbenzenesulfonate (1.75 g, 4.2735 mmol, 57% yield) as a pale yellow solid.

UPLC-MS (ES+, Method 5): 1.88 min, 410.2 [M+H]⁺

¹H NMR (400 MHz, DMSO-d6) δ/ppm: 8.38 (s, 1H), 7.83 (d, 2H), 7.45 (d, 2H), 6.39 (s, 1H), 4.37 (m, 1H), 4.27 (s, 2H), 3.29 (s, 2H), 3.16 (s, 2H), 2.39 (s, 3H), 1.49 (s, 2H), 1.38 (s, 4H), 1.22 (s, 5H)

Step C, tert-butyl 4,6,7,8-tetrahydropyrazolo[1,5-a][1,4]diazepine-5-carboxylate. A stirring solution of 3-[tert-butoxycarbonyl(1H-pyrazol-3-ylmethyl)amino]propyl 4-methylbenzenesulfonate (1.75 g, 4.27 mmol) in DMF (42.735 mL) was flushed with nitrogen and cooled to 0° C. before adding Sodium hydride (60% dispersed in mineral oil) (512.82 mg, 12.82 mmol). The reaction mixture was allowed to warm to room temperature and left to stir overnight. The reaction mixture was diluted with water (30 mL) and extracted with EtOAc (3×15 mL). Organic fractions were combined, dried over Na₂SO₄, filtered and concentrated. The crude material was purified by flash column chromatography (12 g, 0-10% MeOH in DCM), like fractions were combined and concentrated to afford tert-butyl 4,6,7,8-tetrahydropyrazolo[1,5-a][1,4]diazepine-5-carboxylate (812 mg, 3.4218 mmol, 80% yield) as a pale yellow oil.

UPLC-MS (ES+, Method 5): 1.60 min, 238.1 [M+H]⁺

¹H NMR (400 MHz, DMSO-d6) δ/ppm: 7.22 (s, 1H), 6.13 (d, 1H), 4.44-4.33 (m, 4H), 3.63 (s, 2H), 1.72 (s, 2H), 1.33 (s, 9H)

Step D, 5,6,7,8-tetrahydro-4H-pyrazolo[1,5-a][1,4]diazepine (A-10). Trifluoroacetic acid (13.46 mL, 175.73 mmol) was added to a stirring solution of tert-butyl 4,6,7,8-tetrahydropyrazolo[1,5-a][1,4]diazepine-5-carboxylate (417. mg, 1.76 mmol) in DCM (20 mL) and the reaction mixture left to stir at room temperature overnight. The reaction mixture was concentrated and then dissolved in methanol before loading onto an 10 g SCX cartridge. This was washed with water and methanol before eluting with 1M ammonia methanol and concentrated to afford 5,6,7,8-tetrahydro-4H-pyrazolo[1,5-a][1,4]diazepine (210 mg, 1.5307 mmol, 87% yield) as a yellow oil.

¹H NMR (400 MHz, DMSO-d6) δ/ppm: 7.15 (d, J=1.72 Hz, 1H), 6.03 (d, J=1.72 Hz, 1H), 4.29-4.26 (m, 2H), 3.76 (s, 2H), 3.01-2.98 (m, 2H), 1.69-1.64 (m, 2H). Exchangeable proton not seen.

The intermediates in table 4 below were prepared by analogy with A-10 (intermediate scheme 7), replacing 1H-pyrazole-3-carbaldehyde with building block as described in the table entry.

TABLE 4

| | Intermediate Structure/ | ¹H NMR (400 MHz, DMSO- |
|---|---|---|
| Intermediate Building block | Name | d₆) δ/ppm |
| 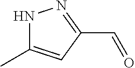 5-methyl-1H-pyrazole-3- carbaldehyde | Intermediate A-20 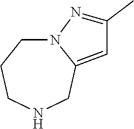 | 5.80 (1H, s), 4.19-4.14 (2H,m), 3.68 (2H, s), 2.99- 2.94 (2H, m), 2.05 (3H, s), 1.67-1.61 (2H, m). |
| | A-20 | |
| | 2-methyl-5,6,7,8-tetrahydro- | |
| | 4H-pyrazolo[1,5- | |
| | a][1,4]diazepine (A-20) | |

### Intermediate A-11, [1-(pyrrolidin-1-ylmethyl)cyclopropyl]methanol

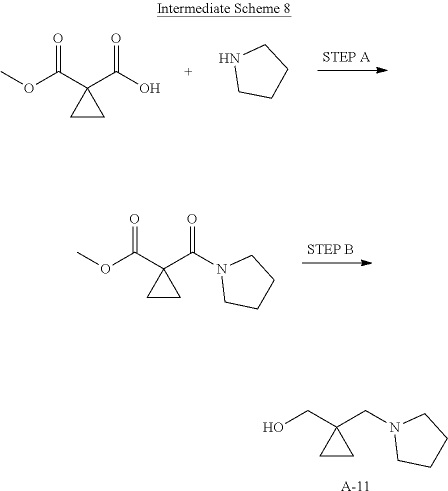

Step A, Methyl 1-(pyrrolidine-1-carbonyl)cyclopropanecarboxylate. To a solution of cyclopropane-1,1-dicarboxylic acid methyl ester (400. mg, 2.78 mmol) and N,N-dimethylformamide (0. mL, 0.03 mmol) in DCM (1.7345 mL) at 0° C. was added oxalyl chloride (0.3 mL, 3.55 mmol). The reaction mixture was left to stir for 3 hours and reduced in vacuo to afford a yellow oil. The residue taken up in THF (1.7345 mL) and cooled to 0° C. before the addition of pyrrolidine (1.19 mL, 2.39 mmol). The mixture was left to stir at room temperature for 2 hours. The reaction mixture was taken up in EtOAc and washed with brine, organics were combined and dried under Na₂SO₄, filtered and the solvents removed in vacuo to afford methyl 1-(pyrrolidine-1-carbonyl)cyclopropanecarboxylate (460 mg, 2.3323 mmol, 84% yield) as a yellow oil.

¹H NMR (400 MHz, DMSO-d6) δ/ppm: 3.65 (s, 3H), 3.37-3.26 (m, 4H), 1.91-1.72 (m, 4H), 1.31-1.28 (m, 2H), 1.26-1.22 (m, 2H).

Step B, [1-(pyrrolidin-1-ylmethyl)cyclopropyl]methanol (A-11). To a solution of methyl 1-(pyrrolidine-1-carbonyl)cyclopropanecarboxylate (460. mg, 2.33 mmol) in dry THF (2.0281 mL) at 0° C. was added Lithium Aluminium hydride 1M in THF (5.13 mL, 5.13 mmol). The reaction mixture was left to stir at room temperature for 2 hours. The reaction mixture was quenched on addition of 2M HCl at 0° C. and the solids filtered. The filtrate was passed through a pre-equilibrated SCX-2 cartridge (2 g, washing with MeOH and eluting with 1M NH₃/MeOH) and the solvents reduced in vacuo to afford [1-(pyrrolidin-1-ylmethyl)cyclopropyl]methanol (110 mg, 0.7086 mmol, 30% yield) as a colourless oil.

¹H NMR (400 MHz, DMSO-d6) δ/ppm: 4.59 (br s, 1H), 2.45-2.39 (m, 4H), 2.35 (s, 2H), 1.69-1.62 (m, 4H), 0.40-0.36 (m, 2H), 0.24-0.20 (m, 2H).

The intermediates in table 5 below were prepared by analogy with A-11 (intermediate scheme 8), using Cyclopropane-1,1-dicarboxylic acid methyl ester and alternative amine building blocks as described in the table entry.

TABLE 5

| | | ¹H NMR (400 |
|---|---|---|
| Intermediate Building | | MHz, DMSO- |
| block | Intermediate Structure/Name | d₆) δ/ppm |
| 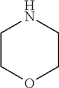 Morpholine | Intermediate A-18 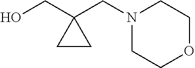 [SMILES: OCC1(CN2CCOCC2)CC1] | 4.46 (s, 1H), 3.58-3.54 (m, 5H), 2.40-2.33 (m, 5H), 2.24 (s, 2H), 0.40 (dd, J = 4.0, 5.9 Hz, 2H), 0.20 |
| | A-18 | (dd, J = 4.1, 5.8 |
| | [1-(morpholino- | Hz, 2H). |
| | methyl)cyclopropyl] | |
| | methanol | |
| 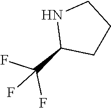 2(S)-2-Trifluoro- methylpyrrolidine | Intermediate A-19 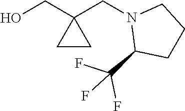 [SMILES: OCC1(CN2CCC[C@H]2C(F)(F)F)CC1] | 4.29-4.26 (m, 1H), 3.40- 3.34 (m, 1H), 3.31-3.27 (m,2H), 3.14- 3.09 (m,2H), 2.42-2.36 (m,1H), 2.22- 2.19 (m, 1H), 2.01-1.91 |
| | A-19 | (m, 1H), 1.79- |
| | [1-[[(2S)-2-(trifluoromethyl)pyrrolidin-1- | 1.67 (m,3H), |
| | yl]methyl]cyclopropyl]methanol | 0.46-0.42 |
| | | (m,1H), 0.39- |
| | | 0.34 (m, 1H), |
| | | 0.32-0.28 |
| | | (m,1H), 0.19- |
| | | 0.14 (m, 1H). |
| 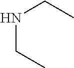 Diethylamine | Intermediate A-32 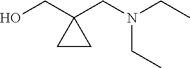 | 4.69 (s, 1H), 2.52-2.46 (m, 6H), 2.37 (s, 2H), 0.93 (t, J = 7.2 Hz, 6H), 0.39-0.21 (m, |
| | A-32 | 4H). |
| | [1-(diethylaminomethyl) | |
| | cyclopropyl]methanol | |
| 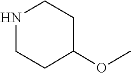 4-Methoxypiperidine | Intermediate A-33 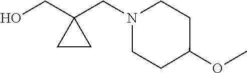 | 4.56 (s, 1H), 3.21 (s, 3H), 3.17-3.09 (m, 1H), 2.77-2.68 (m, 2H), 2.52- 2.49 (m, 2H), 2.23 (s, 2H), |
| | A-33 | 2.08-1.96 (m, |
| | [1-[(4-methoxy-1-piperidyl) | 2H), 1.86-1.74 |
| | methyl]cyclopropyl]methanol | (m, 2H), 1.45- |
| | | 1.31 (m, 2H), |
| | | 0.42-0.17 (m, |
| | | 4H). |

Intermediate A-12, Tert-butyl(2S,4R)-2-(hydroxymethyl)-4-methoxy-pyrrolidine-1-carboxylate.

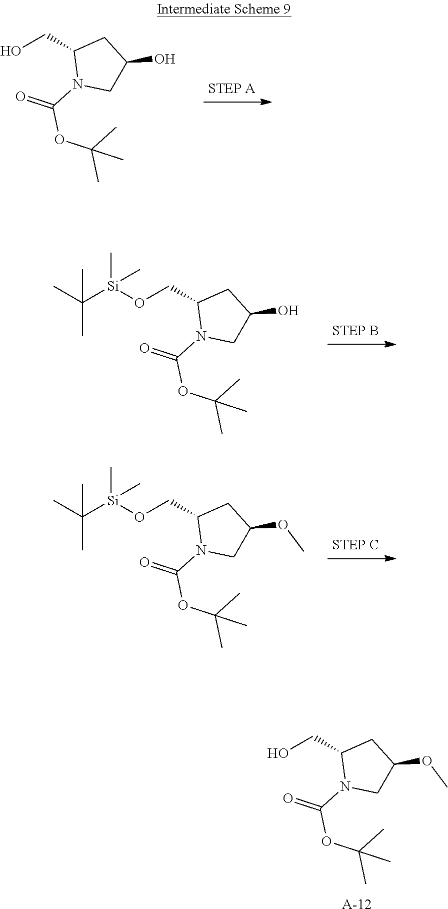

Step A, Tert-butyl(2S,4R)-2-[[tert-butyl(dimethyl) silyl]oxymethyl]-4-hydroxy-pyrrolidine-1-carboxylate. To a solution of tert-butyl(2S,4R)-2-(hydroxymethyl)-4-hydroxy-pyrrolidine-1-carboxylate (500 mg, 2.3 mmol) in DCM (5 mL) at 0° C. was added triethylamine (0.38 mL, 2.76 mmol) and 4-dimethylamino pyridine, DMAP (28.12 mg, 0.23 mmol). The reaction mixture was stirred for 5 mins at 0° C. before the addition of tert-butyl-chlorodimethylsilane (381.55 mg, 2.53 mmol). The reaction mixture was left to stir at room temperature overnight. The mixture was diluted with DCM and washed with sat.aq. NaHCO₃. Organics were combined, dried over Na₂SO₄, filtered and the filtrate evaporated in vacuo to afford tert-butyl(2S,4R)-2-[tert-butyl(dimethyl) silyl]oxymethyl]-4-hydroxy-pyrrolidine-1-carboxylate (763 mg, 2.3015 mmol, 100% yield) as a colourless oil.

¹H NMR (400 MHz, CDCl₃) δ/ppm: 4.55-4.42 (m, 1H), 4.09-3.30 (m, 5H), 2.29-2.15 (m, 1H), 2.11-1.82 (m, 1H), 1.46 (s, 9H), 0.87 (s, 9H), 0.03 (s, 6H). Exchangeable proton not seen.

Step B, Tert-butyl(2S,4R)-2-[[tert-butyl(dimethyl) silyl]oxymethyl]-4-methoxy-pyrrolidine-1-carboxylate. To a solution of tert-butyl(2S,4R)-2-[tert-butyl(dimethyl) silyl]oxymethyl]-4-hydroxy-pyrrolidine-1-carboxylate (763 mg, 2.3 mmol) in DMF (10 mL) at 0° C. was added sodium hydride, (60% dispersed in mineral oil) (119.68 mg, 2.99 mmol). The reaction mixture was stirred for 20 mins at 0° C. before the addition of iodomethane (0.29 mL, 4.6 mmol). The reaction mixture was left to stir at room temperature for 2.5 hours. The reaction mixture was diluted with EtOAc, washed with sat.aq. NH₄Cl, organics combined and washed with water and brine. The organics were combined and dried under Na₂SO₄, filtered and the filtrate evaporated in vacuo to afford a yellow oil. The crude material was purified by flash column chromatography (12 g, eluting in 0-25% EtOAc in pet. ether) and the solvents removed in vacuo to afford tert-butyl(2S,4R)-2-[tert-butyl(dimethyl) silyl]oxymethyl]-4-methoxy-pyrrolidine-1-carboxylate (550 mg, 1.5917 mmol, 69% yield) as a colourless oil.

¹H NMR (400 MHz, CDCl₃) δ/ppm: 4.07-3.83 (m, 3H), 3.73-3.51 (m, 2H), 3.47-3.34 (m, 1H), 3.31 (s, 3H), 2.23-2.11 (m, 1H), 2.09-1.89 (m, 1H), 1.46 (s, 9H), 0.88 (s, 9H), 0.03 (s, 6H).

Step C, Tert-butyl(2S,4R)-2-(hydroxymethyl)-4-methoxy-pyrrolidine-1-carboxylate (A-12). To a stirring solution of tert-butyl(2S,4R)-2-[tert-butyl(dimethyl) silyl]oxymethyl]-4-methoxy-pyrrolidine-1-carboxylate (550 mg, 1.59 mmol) in THF (7.40 mL) was added tetrabutylammonium fluoride 1.0M in THF (3.18 mL, 3.18 mmol). The reaction mixture was left to stir at room temperature for 1 hour. The mixture was reduced in vacuo and the crude residue purified by flash column chromatography (12 g, eluting 0-100% EtOAc in Pet. Ether) to afford the desired product tert-butyl(2S,4R)-2-(hydroxymethyl)-4-methoxy-pyrrolidine-1-carboxylate (300 mg, 1.2971 mmol, 81% yield).

¹H NMR (400 MHz, CDCl₃) δ/ppm: 4.99-4.80 (m, 1H), 4.15-3.99 (m, 1H), 3.93-3.77 (m, 1H), 3.76-3.66 (m, 1H), 3.65-3.50 (m, 2H), 3.43-3.33 (m, 1H), 3.31 (s, 3H), 2.19-2.08 (m, 1H), 1.64-1.54 (m, 1H), 1.47 (s, 9H).

### Intermediate A-13, 8-bromo-6-(methoxymethoxy)tetralin-1-one

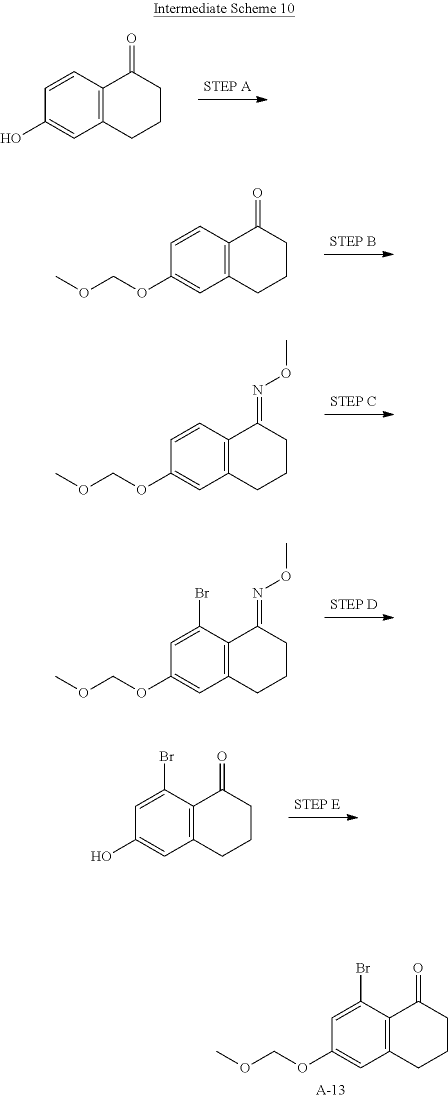

Step A, 6-(methoxymethoxy)tetralin-1-one. To a 0° C. solution of 6-Hydroxy-1-tetralone (3000 mg, 18.5 mmol) and N,N-diisopropylethylamine (4.83 mL, 27.75 mmol) in DCM (36.994 mL) was added bromomethyl methyl ether (1.81 mL, 22.2 mmol). Allowed to stir at that temperature for 30 min. Concentrated under reduced pressure. Partitioned between a layer of ethyl acetate (20 mL) and water (20 mL). Organic layer separated and aqueous layer extracted with ethyl acetate (20 mL). Organic layers combined, washed with a saturated solution of brine (20 mL), dried over sodium sulfate, filtered and concentrated under reduced pressure to afford 6-(methoxymethoxy)tetralin-1-one (3760.8 mg, 18.235 mmol, 99% yield) as a brown oil.

UPLC-MS (ES+, Method 2): 1.53 min, m/z 207.2 [M+H]⁺.

Step B, (E)-N-methoxy-6-(methoxymethoxy)tetralin-1-imine. Triethylamine (7.62 mL, 54.71 mmol) was added to a suspension of methoxyamine hydrochloride (3807.48 mg, 45.59 mmol) and 6-(methoxymethoxy)tetralin-1-one (3760.8 mg, 18.24 mmol) in methanol (36.47 mL). The reaction mixture was heated to 60° C. and left to stir overnight. Additional triethylamine (3.81 mL, 27.35 mmol) and methoxyamine hydrochloride (2284.49 mg, 27.35 mmol) were added and the reaction again left to stir at 60° C. over the weekend. Reaction mixture allowed to cool back down to room temperature and concentrated under reduced pressure. Suspended in ethyl acetate (20 mL), filtered over a celite plug, plug washed with ethyl acetate (2×10 mL), filtrate collected and concentrated under reduced pressure. Purification by flash column chromatography (eluting in 0-40% EtOAc in Pet. Ether) to afford (E)-N-methoxy-6-(methoxymethoxy)tetralin-1-imine (3235.1 mg, 13.75 mmol, 75% yield) as a black oil.

UPLC-MS (ES⁺, Method 2): 2.02 min, m/z 236.4 [M+H]⁺.

Step C, (E)-8-bromo-N-methoxy-6-(methoxymethoxy)tetralin-1-imine. N-Bromosuccinimide (2447.28 mg, 13.75 mmol) and palladium(II) acetate (308.7 mg, 1.38 mmol) were added sequentially to a nitrogen degassed suspension of (E)-N-methoxy-6-(methoxymethoxy)tetralin-1-imine (3235.1 mg, 13.75 mmol) in acetic acid (55 mL). The reaction mixture was heated to 80° C. and left to stir for 30 min. Reaction mixture allowed to cool back down to room temperature, concentrated under reduced pressure and the residue basified with a saturated solution of sodium bicarbonate. Partitioned between a layer of ethyl acetate (40 mL) and water (30 mL). Organic layer separated and aqueous layer extracted with ethyl acetate (40 mL). Organic layers combined, washed with a saturated solution of brine (20 mL), dried over sodium sulfate, filtered and concentrated under reduced pressure to afford (E)-8-bromo-N-methoxy-6-(methoxymethoxy)tetralin-1-imine (4320 mg, 13.75 mmol, 100% yield) as a brown oil.

UPLC-MS (ES⁺, Method 2): 2.16 min, m/z 316.0 [M+H]⁺.

Step D, 8-bromo-6-hydroxy-tetralin-1-one. Hydrogen chloride (11.46 mL, 137.5 mmol) (conc) was added to a solution of (E)-8-bromo-N-methoxy-6-(methoxymethoxy)tetralin-1-imine (4320 mg, 13.75 mmol) in 1,4-dioxane (55 mL). Reaction mixture allowed to stir at 100° C. for 1 h. Reaction mixture allowed to cool back down to room temperature, basified with a saturated solution of sodium bicarbonate and partitioned between a layer of ethyl acetate (80 mL) and water (80 mL). Organic layer was separated and the aqueous layer extracted with ethyl acetate (2×50 mL). Organic layers combined, washed with a saturated solution of brine (40 mL), dried over sodium sulfate, filtered and concentrated under reduced pressure to afford 8-bromo-6-hydroxy-tetralin-1-one (3314.9 mg, 13.75 mmol, 100% yield) as a brown oil.

UPLC-MS (ES⁺, Method 2): 1.39 min, m/z 242.9 [M+H]⁺.

Step E, 8-bromo-6-(methoxymethoxy)tetralin-1-one (A-13). To a solution of 8-bromo-6-hydroxy-tetralin-1-one (3314.9 mg, 13.75 mmol) and N,N-Diisopropylethylamine (4.79 mL, 27.5 mmol) in DCM (27.5 mL) was added Bromomethyl methyl ether (1.35 mL, 16.5 mmol) at 0° C. The reaction mixture was allowed to stir for 30 min and concentrated under reduced pressure. The residue was partitioned between a layer of ethyl acetate (50 mL) and water (50 mL). Organic layer was separated and aqueous layer extracted with ethyl acetate (30 mL). Organic layers combined, washed with a saturated solution of brine (20 mL), dried over sodium sulfate, filtered and concentrated under reduced pressure. The crude material was purified by flash column chromatography (eluting with 0-50% EtOAc in Pet. Ether) to afford 8-bromo-6-(methoxymethoxy)tetralin-1-one (1907.3 mg, 6.689 mmol, 49% yield) as a yellow oil.

UPLC-MS (ES⁺, Method 2): 1.76 min, m/z 286.9 [M+H]⁺.

### Intermediate A-14, 8-bromo-1-ethyl-6-(methoxymethoxy)tetralin

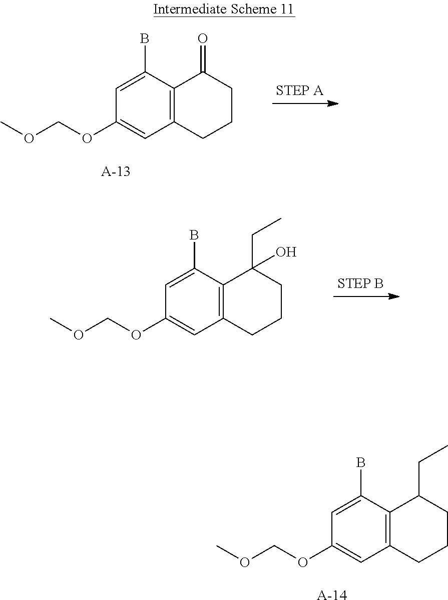

Step A, 8-bromo-1-ethyl-6-(methoxymethoxy)tetralin-1-ol. Ethylmagnesium bromide (7.36 mL, 7.36 mmol) (1M in THF) was added to a 0° C. solution of 8-bromo-6-(methoxymethoxy)tetralin-1-one (700. mg, 2.45 mmol) in THF (4.9099 mL). The reaction mixture was left to stir at 0° C. temperature for 1 h and quenched with a saturated solution of ammonium chloride. Partitioned between a layer of ethyl acetate (20 mL) and water (20 mL). Organic layer separated and aqueous layer extracted with ethyl acetate (20 mL). Organic layers combined, washed with a saturated solution of brine (10 mL), dried over sodium sulfate, filtered and concentrated under reduced pressure. Purification by flash column chromatography (eluting 0-40% EtOAc in Pet. Ether) to afford 8-bromo-1-ethyl-6-(methoxymethoxy)tetralin-1-ol (214.1 mg, 0.6792 mmol, 28% yield) as a brown oil.

UPLC-MS (ES⁺, Method 2): 2.08 min, m/z 298.9 [M+H]⁺.

Step B, 8-bromo-1-ethyl-6-(methoxymethoxy)tetralin (A-14). Trifluoroacetic acid (0.07 mL, 0.95 mmol) was added to a solution of Triethylsilane (0.3 mL, 1.9 mmol) and 8-bromo-1-ethyl-6-(methoxymethoxy)tetralin-1-ol (150. mg, 0.48 mmol) in DCM (2.3794 mL). The reaction mixture was left to stir at room temperature overnight. Basified with a saturated solution of sodium bicarbonate and partitioned between a layer of ethyl acetate (20 mL) and water (20 mL). Organic layer separated and aqueous layer extracted with ethyl acetate (20 mL). Organic layers combined, washed with a saturated solution of brine (10 mL), dried over sodium sulfate, filtered and concentrated under reduced pressure. Purification by flash column chromatography (eluting with 0-30% EtOAc in Pet. Ether) to afford 8-bromo-1-ethyl-6-(methoxymethoxy)tetralin (103.3 mg, 0.3452 mmol, 73% yield) as a clear oil.

¹H NMR (400 MHz, CDCl₃) δ/ppm: 7.12 (d, J=2.6 Hz, 1H), 6.71 (d, J=2.5 Hz, 1H), 5.11 (s, 2H), 3.46 (s, 3H), 2.86-2.65 (m, 3H), 2.02-1.92 (m, 1H), 1.86-1.54 (m, 4H), 1.41-1.24 (m, 1H), 1.00 (t, J=7.4 Hz, 3H).

### Intermediate A-15, (8-ethyl-7-fluoro-1-naphthyl)trifluoromethanesulfonate

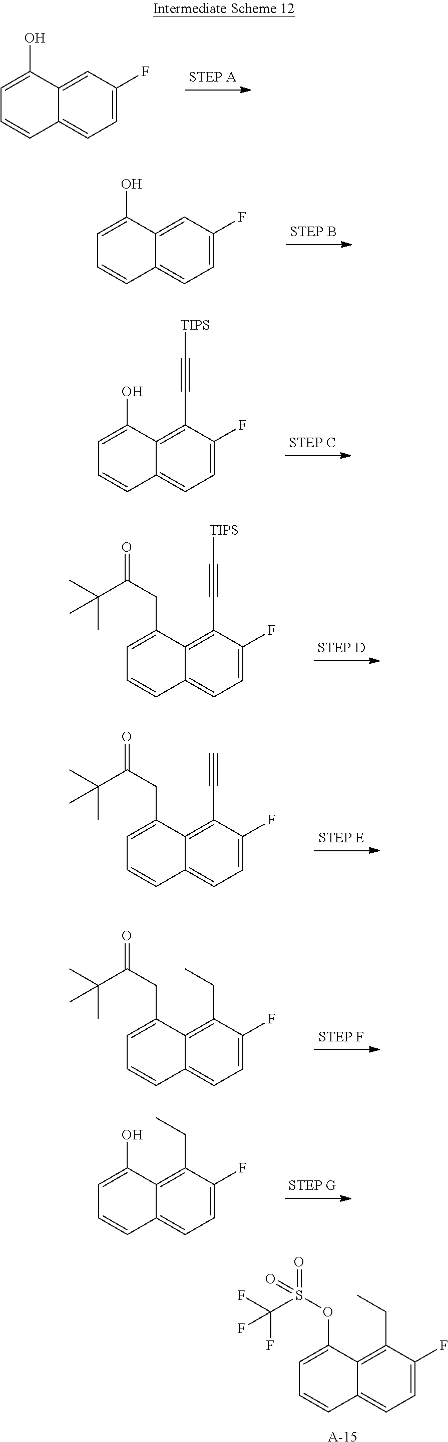

Step A, 7-fluoronaphthalen-1-ol. A solution of 7-Fluoro-3,4-dihydronaphthalen-1 (2H)-one (500. mg, 3.05 mmol) in acetic acid (10 mL) was cooled to 0° C. and HBr (24.64 mg, 0.3 mmol) added. A solution of bromine (535.36 mg, 3.35 mmol) in acetic acid (1 mL) was added to the mixture. The reaction mixture was warmed to room temperature and left to stir for 3 h. The reaction mixture was diluted with water (100 mL) and extracted with DCM (40 mL×3). The combined organic layers were washed with brine (100 mL), dried over Na₂SO₄ and concentrated in vacuo. The residue was taken up in DMF (10 mL), lithium bromide (449.64 mg, 5.18 mmol) and lithium carbonate (382.55 mg, 5.18 mmol) were added. The mixture was heated to 160° C. and left to stir for 3.5 hrs. The reaction mixture was diluted with water (150 mL) and extracted with EtOAc (60 mL×3). The combined organic layers were washed with brine (200 mL), dried over Na₂SO₄ and concentrated in vacuo. The crude material was purified by flash column chromatography to afford 7-fluoronaphthalen-1-ol (270 mg, 1.665 mmol, 55% yield) as a brown solid.

¹H NMR (400 MHz, DMSO-d6) δ/ppm: 10.27 (s, 1H), 7.69 (dd, J=9.0, 5.8 Hz, 1H), 7.78 (dd, J=10.8, 2.5 Hz, 1H), 7.47-7.42 (m, 2H), 7.37-7.30 (m, 1H), 6.99 (d, J=7.6 Hz, 1H).

Step B, 7-fluoro-8-(2-triisopropylsilylethynyl)naphthalen-1-ol. To a stirring solution of 7-fluoronaphthalen-1-ol (270. mg, 1.67 mmol) in 1,4-dioxane (5 mL) was added potassium acetate (326.81 mg, 3.33 mmol), 1-isopropyl-4-methylbenzene dimer (101.96 mg, 0.17 mmol) and (bromoethynyl)triisopropylsilane (0.43 mL, 1.75 mmol). The reaction mixture was heated to 110° C. and left to stir for 2 hours. The reaction mixture was cooled to room temperature, filtered and concentrated in vacuo. The crude material was purified by flash column chromatography to afford 7-fluoro-8-(2-triisopropylsilylethynyl)naphthalen-1-ol (470 mg, 1.3721 mmol, 82% yield) as a yellow oil.

¹H NMR (400 MHz, DMSO-d6) δ/ppm: 10.12 (s, 1H), 7.92 (dd, J=9.0, 5.9 Hz, 1H), 7.43 (d, J=9.0 Hz, 1H), 7.40 (d, J=8.2 Hz, 1H), 7.36-7.28 (m, 1H), 6.97 (d, J=7.5 Hz, 1H), 1.23-1.11 (m, 21H).

Step C, [7-fluoro-8-(2-triisopropylsilylethynyl)-1-naphthyl] 2,2 dimethylpropanoate. To a stirring solution of 7-fluoro-8-(2-triisopropylsilylethynyl)naphthalen-1-ol (470. mg, 1.37 mmol) in DCM (5 mL) at 0° C. was added triethylamine (416.54 mg, 4.12 mmol) and 4-dimethylaminopyridine (33.53 mg, 0.27 mmol). The reaction mixture was stirred at 0° C. for 5 mins. Trimethyl acetyl chloride (0.51 mL, 4.12 mmol) was added, the reaction mixture warmed to room temperature and left to stir for 1 h. The reaction mixture was diluted with sat. aq. NH₄Cl (100 mL) and extracted with EtOAc (30 mL×4). The combined organic layers were washed with brine (200 mL×3), dried over Na₂SO₄ and concentrated in vacuo. The crude material was purified by flash column chromatography (eluting in Pet.ether/EtOAc=5/1) to afford [7-fluoro-8-(2-triisopropylsilylethynyl)-1-naphthyl] 2,2-dimethylpropanoate (560 mg, 1.3126 mmol, 96% yield) as a yellow solid.

¹H NMR (400 MHz, DMSO-d6) δ/ppm: 8.20 (dd, J=9.0, 5.8 Hz, 1H), 8.00 (d, J=8.2 Hz, 1H), 7.69-7.58 (m, 2H), 7.29 (d, J=7.60, Hz, 1H), 1.46 (s, 9H), 1.23-1.11 (m, 21H).

Step D, (8-ethynyl-7-fluoro-1-naphthyl) 2,2-dimethylpropanoate. CsF (35.6 g, 234.38 mmol) was added to a stirring solution of [7-fluoro-8-(2-triisopropylsilylethynyl)-1-naphthyl] 2,2-dimethylpropanoate (10. g, 23.44 mmol) in Methanol (100 mL) and the reaction mixture left to stir at room temperature for 30 mins. The reaction mixture was diluted with water (1000 mL) and extracted with EtOAc (200 mL×3). The combined organic layers were washed with brine (1000 mL), dried over Na₂SO₄ and concentrated in vacuo. The crude material was purified by flash column chromatography to afford (8-ethynyl-7-fluoro-1-naphthyl) 2,2-dimethylpropanoate (4.8 g, 17.758 mmol, 76% yield) as a brown oil.

¹H NMR (400 MHz, CDCl₃) δ/ppm: 7.86 (dd, J=9.0, 5.7 Hz, 1H), 7.72 (d, J=8.2 Hz, 1H), 7.49-7.41 (m, 1H), 7.34-7.24 (m, 1H), 7.11 (d, J=7.4 Hz, 1H), 3.60 (s, 1H), 1.47 (s, 9H).

Step E, (8-ethyl-7-fluoro-1-naphthyl) 2,2-dimethylpropanoate. A stirring solution of (8-ethynyl-7-fluoro-1-naphthyl) 2,2-dimethylpropanoate (3.8 g, 14.06 mmol) in Methanol (100 mL) was degassed and backfilled with nitrogen three times before Pd/C (380 mg) was added. The stirring mixture was degassed and backfilled with hydrogen, the mixture warmed to 40° C. and left to stir for 2 hours. The reaction mixture was cooled to room temperature before being degassed and backfilled with nitrogen then filtered through a pad of celite, washing with MeOH and the filtrate reduced in vacuo to afford the desired product (8-ethyl-7-fluoro-1-naphthyl) 2,2-dimethylpropanoate (2.8 g, 10.206 mmol, 73% yield) as a brown oil.

¹H NMR (400 MHz, DMSO-d6) δ/ppm: 7.94 (dd, J=8.9, 5.9 Hz, 1H), 7.88 (d, J=8.1 Hz, 1H), 7.52-7.41 (m, 2H), 7.15 (d, J=7.6 Hz, 1H), 3.12-3.01 (m, 2H), 1.40 (s, 9H), 1.16 (t, J=7.5 Hz, 3H).

Step F, 8-ethyl-7-fluoro-naphthalen-1-ol. To a solution of (8-ethyl-7-fluoro-1-naphthyl) 2,2-dimethylpropanoate (950. mg, 3.46 mmol) in Methanol (10 mL) was added potassium hydroxide (777.2 mg, 13.85 mmol) and the mixture stirred at room temperature for 30 mins. The reaction mixture was diluted with water (100 mL) and extracted with DCM (20 mL×3). The combined organic layers were washed with brine (100 mL), dried over Na₂SO₄ and concentrated in vacuo. The crude material was purified by flash column chromatography to afford 8-ethyl-7-fluoro-naphthalen-1-ol (250 mg, 1.3143 mmol, 38% yield) as a brown oil.

¹H NMR (400 MHz, DMSO-d6) δ/ppm: 10.06 (s, 1H), 7.72 (dd, J=9.0, 6.0 Hz, 1H), 7.39-7.21 (m, 3H), 6.92 (d, J=7.5 Hz, 1H), 3.40-3.30 (m, 2H), 1.25 (t, J=7.3 Hz, 3H).

Step G, (8-ethyl-7-fluoro-1-naphthyl)trifluoromethanesulfonate (A-15). N,N-Diisopropylethylamine (4.08 g, 31.54 mmol) was added to a stirring solution of 8-ethyl-7-fluoro-naphthalen-1-ol (1. g, 5.26 mmol) in DCM (10 mL) at −40° C. and the mixture stirred for 5 mins. trifluoromethanesulfonic anhydride (1.93 g, 6.83 mmol) was added and the mixture was stirred for 30 mins. The reaction mixture was diluted with water (100 mL) and extracted with DCM (40 mL×3). The combined organic layers were washed with brine (100 mL), dried over Na₂SO₄ and concentrated in vacuo. The crude material was purified by flash column chromatography to afford (8-ethyl-7-fluoro-1-naphthyl)trifluoromethanesulfonate (1.5 g, 4.6545 mmol, 89% yield) as a brown oil.

¹H NMR (400 MHz, DMSO-d6) δ/ppm: 8.15 (dd, J=8.0, 1.0 Hz, 1H), 8.09 (dd, J=9.0, 5.9 Hz, 1H), 7.71 (d, J=7.7 Hz, 1H), 7.67-7.58 (m, 2H), 3.26-3.17 (m, 2H), 1.20 (t, J=7.5 Hz, 3H).

### Intermediate A-16, 4-bromo-1-fluoro-2-(methoxymethoxy) naphthalene

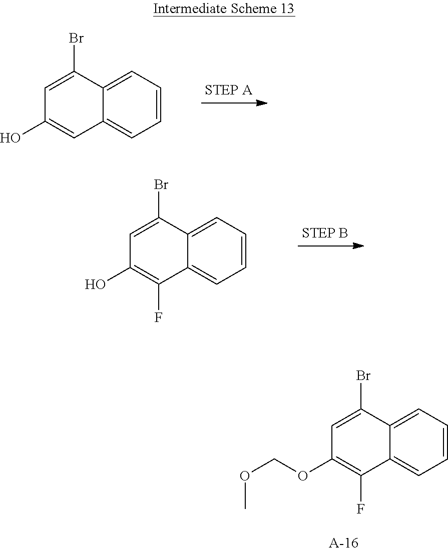

Step A, 4-bromo-1-fluoro-naphthalen-2-ol. To a stirring solution of 1-Bromo-3-hydroxynaphthalene (500. mg, 2.24 mmol) in MeCN (20 mL) at 0′C was added Selectfluor (794.06 mg, 2.24 mmol). The mixture was stirred at 0′C to 15′C (ice bath) overnight. Additional Selectfluor (397.03 mg, 1.12 mmol) was added and the mixture was left to stir for 2 hrs. The reaction mixture was quenched with sat. NaHCO₃ solution and extracted with DCMx3. The combined organic layers were filtered through a phase separator cartridge and concentrated in vacuo to afford 4-bromo-1-fluoro-naphthalen-2-ol (540.32 mg, 2.2414 mmol, 100% yield) as a yellow oil.

UPLC-MS (ES−, Method 5): 2.02 min, m/z 238.9 240.9 [M−H].

Step B, 4-bromo-1-fluoro-2-(methoxymethoxy) naphthalene (A-16). A stirring solution of 4-bromo-1-fluoro-naphthalen-2-ol (540. mg, 1.39 mmol) in DCM (12 mL) was cooled to 0° C. and N,N-Diisopropylethylamine (0.48 mL, 2.78 mmol) and Bromomethyl methyl ether (0.12 mL, 1.53 mmol) were added. The reaction mixture was stirred at 0° C. for 2 hrs. The reaction mixture was quenched with water and extracted with DCMx3. The combined organics were filtered through a phase separator cartridge and concentrated in vacuo. The crude material was purified by flash column chromatography (12 g, eluting in 0-20% EtOAc in Pet. ether) to afford 4-bromo-1-fluoro-2-(methoxymethoxy) naphthalene (189 mg, 0.5635 mmol, 41% yield) as a yellow oil.

¹H NMR (400 MHz, CDCl₃) δ/ppm: 8.16-8.11 (m, 1H), 8.08-8.04 (m, 1H), 7.75 (d, J=7.4 Hz, 1H), 7.60-7.51 (s, 2H), 5.29 (s, 2H), 3.58 (s, 3H).

### Intermediate A-17, [(4R)-4-[tert-butyl(dimethyl) silyl]oxy-1,2-dimethyl-pyrrolidin-2-yl]methanol

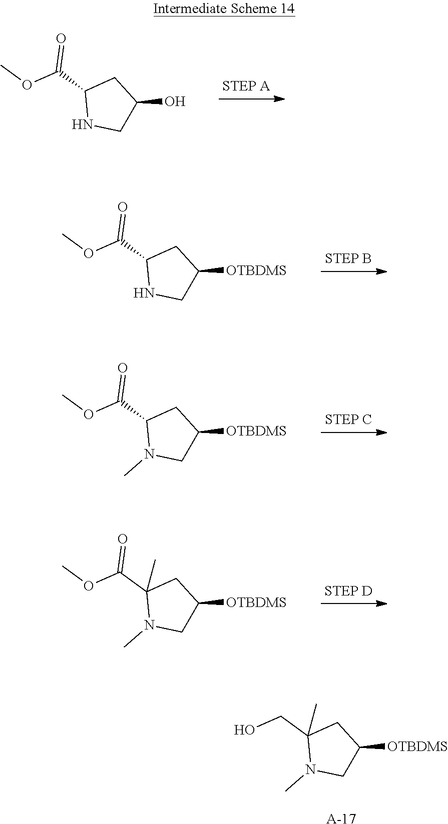

Step A, methyl(2S,4R)-4-[tert-butyl(dimethyl) silyl]oxypyrrolidine-2-carboxylate. To a solution of L-4-Hydroxyproline methyl ester hydrochloride (300 mg, 2.07 mmol) in DCM (8 mL) at 25° C. was added Tert-butyl-chlorodimethylsilane purum (373.8 mg, 2.48 mmol) and imidazole (281.4 mg, 4.13 mmol). The mixture was stirred at 25° C. for 18 hrs. Reaction mixture was quenched with water and the layers separated. The aqueous layer was extracted x3 with DCM. Combined organics were washed with brine, passed through an hydrophobic frit and concentrated under reduced pressure to yield methyl(2S,4R)-4-[tert-butyl(dimethyl) silyl]oxypyrrolidine-2-carboxylate (390 mg, 1.50 mmol, 73% yield) as a clear oil.

¹H NMR (400 MHz, CDCl₃) δ/ppm: 4.41-4.34 (m, 1H), 4.00 (t, J=8.0 Hz, 1H), 3.73 (s, 3H), 3.11 (dd, J=11.5, 4.5 Hz, 1H), 2.87-2.81 (m, 1H), 2.10-2.02 (m, 1H), 1.99-1.89 (m, 2H), 0.88 (s, 9H), 0.06 (s, 3H), 0.05 (s, 3H).

Step B, methyl(2S,4R)-4-[tert-butyl(dimethyl) silyl]oxy-1-methyl-pyrrolidine-2-carboxylate. To a solution of methyl(2S,4R)-4-[tert-butyl(dimethyl) silyl]oxypyrrolidine-2-carboxylate (190 mg, 0.73 mmol) in Methanol (5 mL) was added formaldehyde solution (36.5-38%) in water (0.3 mL, 3.66 mmol). The mixture was stirred at 25° C. for 10 mins then sodium triacetoxyborohydride (232.84 mg, 1.1 mmol) was added. Allowed to stir for 2 hrs at 25° C. Reaction mixture was concentrated in vacuo, taken up in DCM and washed with NaHCO₃ saturated aqueous solution x2 and brine. The resulting organics were passed through a hydrophobic frit and reduced in vacuo to yield methyl (2S,4R)-4-[tert-butyl(dimethyl) silyl]oxy-1-methyl-pyrrolidine-2-carboxylate (132 mg, 0.48 mmol, 66% yield) as an orange oil.

¹H NMR (400 MHz, CDCl₃) δ/ppm: 4.45-4.38 (m, 1H), 3.73 (s, 3H), 3.37 (dd, J=9.6, 6.0 Hz, 1H), 3.26 (t, J=8.3 Hz, 1H), 2.40 (s, 3H), 2.30 (dd, J=9.6, 5.5 Hz, 1H), 2.24-2.12 (m, 1H), 2.07-1.96 (m, 1H), 0.88 (s, 9H), 0.04 (s, 3H), 0.03 (s, 3H).

Step C, methyl (4R)-4-[tert-butyl(dimethyl) silyl]oxy-1,2-dimethyl-pyrrolidine-2-carboxylate. Diisopropylamine (1.54 mL, 10.97 mmol) was dissolved in dry THF (30 mL) and cooled to −78° C. n-Butyllithium solution (6.86 mL, 10.97 mmol) was added, and the mixture was stirred for 30 mins at −78° C. At −78° C., methyl(2S,4R)-4-[tert-butyl(dimethyl) silyl]oxy-1-methyl-pyrrolidine-2-carboxylate (1.5 g, 5.49 mmol) in THF (10 mL) was added dropwise. Temperature was maintained at −78° C. for 5 min before warming to 0° C. After 1 hr, Iodomethane (1.02 mL, 16.46 mmol) was added at 0° C. and reaction mixture was allowed to warm to 25° C. and stir for 18 hrs. The reaction mixture was quenched with a saturated aqueous solution of NH₄Cl. EtOAc was added and the layers separated. The aqueous layer was extracted with EtOAc x3. Organic layers were combined, dried over Na₂SO₄ and filtered. The filtrate was concentrated under reduced pressure. Crude residue was purified by flash chromatography (12 g, dry-load onto HM-N absorbant, 0-80% EtOAc in Petroleum ether, detected using TLC). Relevant fractions were pooled and concentrated to yield methyl rac-(4R)-4-[tert-butyl(dimethyl) silyl]oxy-1,2-dimethyl-pyrrolidine-2-carboxylate (493 mg, 1.7149 mmol, 31% yield) as a yellow oil.

¹H NMR (400 MHz, CDCl₃) δ/ppm: 4.47-4.40 (m, 0.75H), 4.40-4.34 (m, 0.25H), 3.70 (s, 1H), 3.68 (s, 2H), 3.09 (dd, J=9.7, 6.7 Hz, 1H), 2.90 (dd, J=9.4, 4.8 Hz, 0.25H), 2.77 (dd, J=9.7, 3.5 Hz, 0.75H), 2.52 (dd, J=13.3, 7.8 Hz, 0.75H), 2.37 (s, 1H), 2.28 (s, 2H), 2.25 (dd, J=13.2, 4.4 Hz, 0.25H), 2.05-1.98 (m, 0.25H), 1.70 (dd, J=13.3, 4.6 Hz, 0.75H), 1.35 (s, 2H), 1.27 (s, 1H), 0.87 (s, 6H), 0.86 (s, 3H), 0.04 (s, 4H), 0.02 (s, 2H).

Step D, [(4R)-4-[tert-butyl(dimethyl) silyl]oxy-1,2-dimethyl-pyrrolidin-2-yl]methanol (A-17). Lithium aluminium hydride (3.77 mL, 3.77 mmol) was added to a solution of methyl (4R)-4-[tert-butyl(dimethyl) silyl]oxy-1,2-dimethyl-pyrrolidine-2-carboxylate (493. mg, 1.71 mmol) in dry THF (7 mL) at 0° C. Mixture was allowed to warm to 25° C. and stirred for 2 hrs. The reaction mixture was quenched with MeOH, water and Rochelle's salt, and filtered. EtOAc was added and the layers were separated. The aqueous layer was extracted with EtOAc x3. Combined organics were washed with brine, passed through hydrophobic filter paper and concentrated under reduced pressure to yield [(4R)-4-[tert-butyl(dimethyl) silyl]oxy-1,2-dimethyl-pyrrolidin-2-yl]methanol (285 mg, 1.10 mmol, 64% yield) (A-17) as an orange oil.

¹H NMR (400 MHz, CDCl₃) δ/ppm: 4.28-4.20 (m, 1H), 3.27-3.20 (m, 2H), 3.14 (d, J=10.4 Hz, 1H), 2.54 (dd, J=9.2, 6.6 Hz, 1H), 2.33-2.23 (m, 1H), 2.16 (s, 3H), 1.45 (dd, J=13.2, 4.7 Hz, 1H), 0.99 (s, 3H), 0.87 (s, 9H), 0.04-0.02 (m, 6H).

### Intermediate A-37, tert-butyl (4R)-2-(hydroxymethyl)-2-methyl-4-tetrahydropyran-2-yloxy-pyrrolidine-1-carboxylate

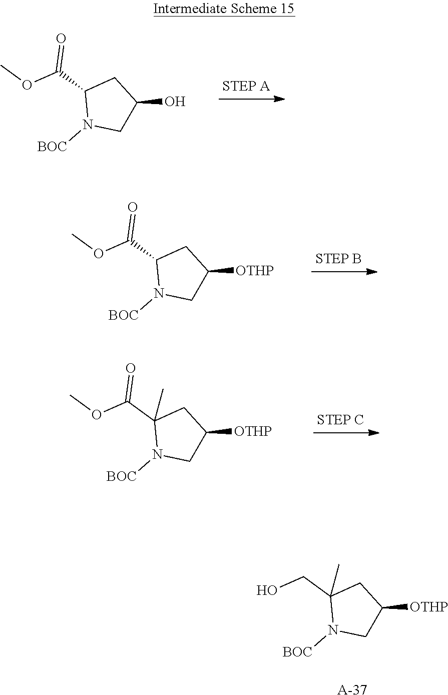

Step A, O1-tert-butyl O2-methyl(2S,4R)-4-tetrahydropyran-2-yloxypyrrolidine-1,2-dicarboxylate. To a solution of n-boc-trans-4-hydroxy-I-proline methyl ester (1000 mg, 4.08 mmol) in DCM (10 mL) at 0° C. was added p-toluenesulfonic acid monohydrate (77.55 mg, 0.41 mmol) followed by 3,4-dihydro-2H-pyran (1.12 mL, 12.23 mmol). The reaction mixture was stirred at room temperature overnight. for overnight. The reaction mixture was reduced in vacuo to afford a black/brown oily residue. Crude material was purified by flash column chromatography (25 g column, eluting 0-60% EtOAc in pet ether) to afford the desired product O1-tert-butyl O2-methyl (2S,4R)-4-tetrahydropyran-2-yloxypyrrolidine-1,2-dicarboxylate (1.056 g, 3.2059 mmol, 79% yield) as an yellow oil.

UPLC-MS (ES⁺, Method 2): 1.76 min, m/z 352.2 [M+Na]⁺ and m/z 230.1 [M-Boc]⁺

Step B, O1-tert-butyl 02-methyl rac-(4R)-2-methyl-4-tetrahydropyran-2-yloxy-pyrrolidine-1,2-dicarboxylate. To a solution of 01-tert-butyl O2-methyl(2S,4R)-4-tetrahydropyran-2-yloxypyrrolidine-1,2-dicarboxylate (335. mg, 1.02 mmol) in dry THF (5 mL) under N₂ was added lithium diisopropylamide (0.61 mL, 1.22 mmol) dropwise at −78° C. The reaction mixture was stirred for 1 hour and Iodomethane (0.09 mL, 1.53 mmol) was added dropwise. The reaction mixture was then allowed to warm to room temperature and left to stir overnight. The reaction mixture was quenched with sat. NH₄Cl (aq), EtOAc added and the layers separated. The aqueous layer further extracted with EtOAc (×3). The organic layers were combined, dried over Na₂SO₄, filtered and the solvents removed in vacuo. The crude material was purified by flash chromatography (12 g, eluting in 0-50% EtOAa in Pet. Ether, ELS detector) to afford the desired product O1-tert-butyl O2-methyl (4R)-2-methyl-4-tetrahydropyran-2-yloxy-pyrrolidine-1,2-dicarboxylate (241 mg, 0.7018 mmol, 69% yield) as a yellow oil.

UPLC-MS (ES⁺, Method 5): 2.04 min, m/z 366.3 [M+Na]⁺

Step C, tert-butyl (4R)-2-(hydroxymethyl)-2-methyl-4-tetrahydropyran-2-yloxy-pyrrolidine-1-carboxylate (A-37). Lithium borohydride (38.21 mg, 1.75 mmol) was added to a solution of O1-tert-butyl 02-methyl (4R)-2-methyl-4-tetrahydropyran-2-yloxy-pyrrolidine-1,2-dicarboxylate (241. mg, 0.7 mmol) in THF (4 mL) at 0° C. The reaction mixture was allowed to warm to room temperature and left to stir overnight. The reaction mixture was quenched with MeOH and concentrated in vacuo. The crude residue was purified by flash chromatography (4 g, eluting in 0-50% EtOAa in Pet. ether) to afford the desired product tert-butyl (4R)-2-(hydroxymethyl)-2-methyl-4-tetrahydropyran-2-yloxy-pyrrolidine-1-carboxylate (198 mg, 0.6278 mmol, 89.454% yield) as a colourless oil.

UPLC-MS (ES⁺, Method 5): 1.84 min, m/z 316.1 [M+H]⁺

Intermediate A-31, [1-(methoxymethyl)-2-oxabicyclo[2.1.1]hexan-4-yl]methanol.

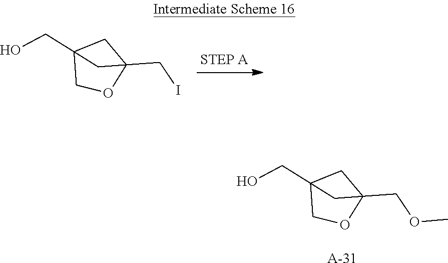

Step A, [1-(methoxymethyl)-2-oxabicyclo[2.1.1]hexan-4-yl]methanol (A-31). Sodium methoxide (0.73 mL, 3.94 mmol) (5.4M in MeOH) was added to a solution of [1-(iodomethyl)-2-oxabicyclo[2.1.1]hexan-4-yl]methanol (100. mg, 0.39 mmol) in Methanol (1.968 mL). The reaction mixture was heated to 60° C. and left to stir overnight. Reaction mixture allowed to cool back down to room temperature. Partitioned between a layer of ethyl acetate (20 mL) and water (20 mL). Organic layer separated and aqueous layer extracted with ethyl acetate (20 mL). Organic layers combined, washed with a saturated solution of brine (10 mL), dried over sodium sulfate, filtered and concentrated under reduced pressure to afford [1-(methoxymethyl)-2-oxabicyclo[2.1.1]hexan-4-yl]methanol (36.4 mg, 0.2301 mmol, 58% yield) as a yellow oil.

¹H NMR (400 MHz, CDCl₃) δ/ppm: 3.91 (s, 2H), 3.75 (s, 2H), 3.47 (s, 2H), 3.42 (s, 3H), 1.76-1.71 (m, 2H), 1.68-1.62 (m, 2H), 1.39 (br s, 1H).

### Intermediate A-21, 5,6,7,8-tetrahydro-4H-pyrazolo[1,5-a][1,4]diazepine-2-carbonitrile

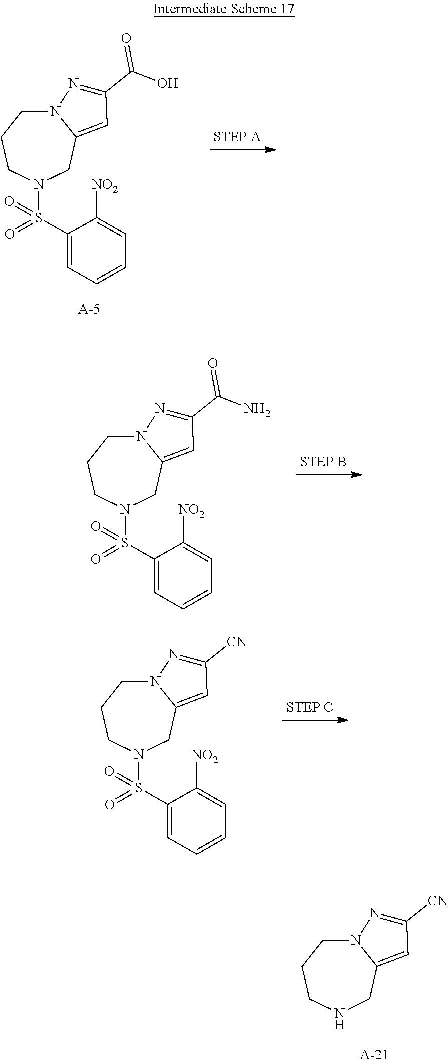

Step A, 5-(2-nitrophenyl) sulfonyl-4,6,7,8-tetrahydropyrazolo[1,5-a][1,4]diazepine-2-carboxamide. HATU (856.3 mg, 2.25 mmol) was added to a solution of ammonium chloride (1095.1 mg, 20.47 mmol), N,N-Diisopropylethylamine (1.07 mL, 6.14 mmol) and 5-(2-nitrophenyl) sulfonyl-4,6,7,8-tetrahydropyrazolo[1,5-a][1,4]diazepine-2-carboxylic acid (750 mg, 2.05 mmol) in dry DMF (4.0944 mL) and stirred at 25° C. overnight. Further N,N-Diisopropylethylamine (0.53 mL, 3.07 mmol) and HATU (311.37 mg, 0.82 mmol) were added and stirred at 25° C. overnight again. Mixture was acidified to pH2, diluted with water and EtOAc and the aqueous was extracted with EtOAc. Combined organics washed with brine and dried under Na₂SO₄, filtered, filtrate evaporated in vacuo. Residue was Suspended in EtOAc/MeOH and cream solid was filtered off and dried under vacuum affording some of the product. Filtrate was purified by flash chromatography (eluting 0-15% MeOH in EtOAc). The two batches were combined to afford the desired product 5-(2-nitrophenyl) sulfonyl-4,6,7,8-tetrahydropyrazolo[1,5-a][1,4]diazepine-2-carboxamide (684 mg, 1.872 mmol, 91% yield) as a beige solid.

¹H NMR (400 MHz, DMSO-d6) δ/ppm: 8.04-7.97 (2H, m), 7.92-7.87 (1H, m), 7.85-7.79 (1H, m), 7.42 (1H, s) 7.18 (1H, s), 6.60 (1H, s), 4.66 (2H, s), 4.48-4.39 (2H, m), 3.71-3.61 (2H, m), 1.97-1.87 (2H, m).

Step B, 5-(2-nitrophenyl) sulfonyl-4,6,7,8-tetrahydropyrazolo[1,5-a][1,4]diazepine-2-carbonitrile. 5-(2-nitrophenyl) sulfonyl-4,6,7,8-tetrahydropyrazolo[1,5-a][1,4]diazepine-2-carboxamide (408.5 mg, 1.12 mmol) was dissolved in DCM (11.2 mL) and Triethylamine (0.47 mL, 3.35 mmol) then cooled to 0° C. Trifluoroacetic anhydride (0.31 mL, 2.24 mmol) was added dropwise and stirred at 0° C. for 1 hr. Diluted with DCM, washed twice with aqueous saturated NaHCO₃ solution, passed through a hydrophobic filter paper and concentrated in vacuo affording 5-(2-nitrophenyl) sulfonyl-4,6,7,8-tetrahydropyrazolo[1,5-a][1,4]diazepine-2-carbonitrile (611.5 mg, 1.7605 mmol, 99% yield) as a yellow/orange gum. Material was used without further purification.

¹H NMR (400 MHz, CDCl₃) δ/ppm: 8.04-7.99 (1H, m), 7.77-7.64 (3H, m), 6.64 (1H, s), 4.57 (2H, s), 4.52-4.45 (2H, m), 3.76-3.65 (2H, m), 2.15-2.05 (2H, m).

Step C, 5,6,7,8-tetrahydro-4H-pyrazolo[1,5-a][1,4]diazepine-2-carbonitrile (A-21). To 5-(2-nitrophenyl) sulfonyl-4,6,7,8-tetrahydropyrazolo[1,5-a][1,4]diazepine-2-carbonitrile (611. mg, 1.76 mmol) and Cesium Carbonate (1.15 g, 3.52 mmol) in MeCN (8.80 mL) was added Thiophenol (0.36 mL, 3.52 mmol) and the reaction was stirred at room temperature for 90 mins in a sealed vial. The reaction was diluted with EtOAc, passed through a plug of celite, plug washed with EtOAc, the filtrate was collected and concentrated under reduced pressure. Purification by flash column chromatography on silica gel eluting with 0-20% MeOH in DCM afforded 5,6,7,8-tetrahydro-4H-pyrazolo[1,5-a][1,4]diazepine-2-carbonitrile (81.4 mg, 0.5018 mmol, 29% yield) as a pale yellow gum.

¹H NMR (400 MHz, CDCl₃) δ/ppm: 6.46 (s, 1H), 4.50-4.40 (m, 2H), 3.92 (s, 2H), 3.27-3.17 (m, 2H), 1.93-1.83 (m, 2H). Exchangeable proton not seen.

### Intermediate A-22, 5-cyclopropyl-6-methyl-1-tetrahydropyran-2-yl-4-(4,4,5,5-tetramethyl-1,3,2-dioxaborolan-2-yl) indazole

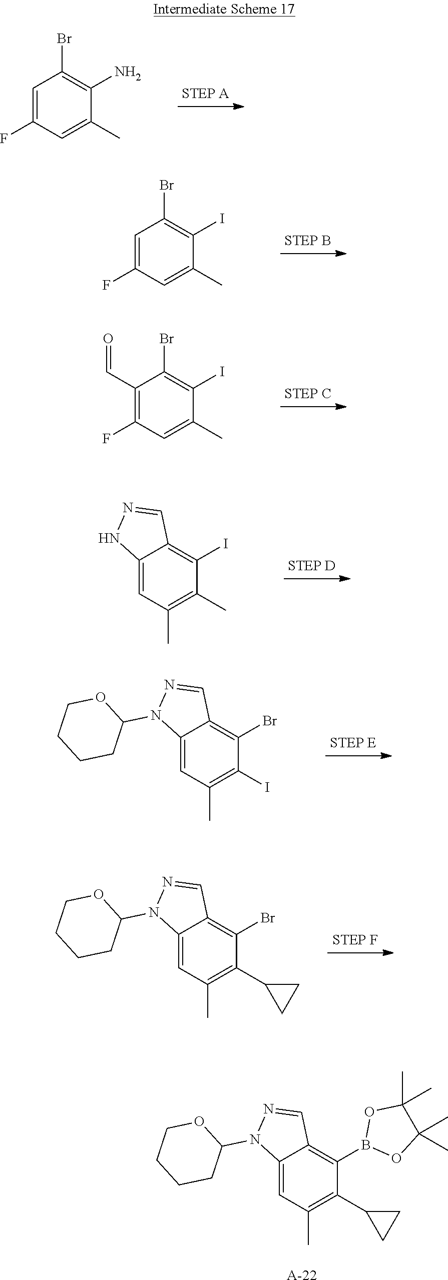

Step A, 1-bromo-5-fluoro-2-iodo-3-methyl-benzene. 2-bromo-4-fluoro-6-methylaniline (4000 mg, 19.6 mmol) was stirred at 70° C. with Hydrogen Chloride (6M in water) (40 mL, 240 mmol) for 30 minutes until completely dissolved. The mixture was cooled to −5° C., and a solution of Sodium nitrite (1623 mg, 23.52 mmol) in Water (10 mL) was added. The mixture was stirred for 30 minutes, then a solution of Potassium iodide (3905.1 mg, 23.52 mmol) in Water (16 mL) was added. The mixture was diluted with EtOAc (2×40 mL), and washed with brine (3×60 mL). The combined organics were dried with Na₂SO₄, filtered and concentrated to give a black solid. The crude was passed through a silica plug, eluting with 20% EtOAc in petroleum ether. The desired fractions were washed with aqueous saturated Na₂S₂O₃, dried and concentrated to afford 1-bromo-5-fluoro-2-iodo-3-methyl-benzene (4653 mg, 14.8 mmol, 75% yield) as a pale orange solid.

¹H NMR (400 MHz, CDCl₃) δ/ppm: 7.26-7.23 (m, 1H), 6.97-6.92 (m, 1H), 2.55 (s, 3H).

Step B, 2-bromo-6-fluoro-3-iodo-4-methyl-benzaldehyde. A solution of 1-bromo-5-fluoro-2-iodo-3-methyl-benzene (4653 mg, 14.8 mmol) in anhydrous THF (17.6 mL) under N₂, was cooled to −78° C., and Lithium diisopropylamide (2M in THF/heptane/ethylbenzene) (8.87 mL, 17.7 mmol) was added slowly at −78° C., the mixture was stirred for 30 minutes. Ethyl formate (0.93 mL, 16.99 mmol) was then added, and stirred at −78° C. for 15 minutes. The mixture was then poured into aqueous saturated NH₄Cl and extracted with EtOAc. The combined organics were dried over Na₂SO₄, filtered and concentrated to afford 2-bromo-6-fluoro-3-iodo-4-methyl-benzaldehyde (5066 mg, 14.8 mmol, 100% yield) a pale orange solid, material was used without further purification.

¹H NMR (400 MHz, CDCl₃) δ/ppm: 10.19 (s, 1H), 7.1 (d, J=10.9 Hz, 1H), 2.63 (s, 3H).

Step C, 4-bromo-5-iodo-6-methyl-1H-indazole. Hydrazine hydrate (4.26 mL, 87.5 mmol) was added to 2-bromo-6-fluoro-3-iodo-4-methyl-benzaldehyde (5000 mg, 14.6 mmol) in DMSO (50 mL) at room temperature under N₂. The mixture was heated to 120° C. overnight. The reaction was cooled to room temperature and diluted with water (50 mL), then extracted with EtOAc (3×100 mL). The combined organics were washed twice with brine, dried with Na₂SO₄, filtered and concentrated to give 4-bromo-5-iodo-6-methyl-1H-indazole (4911 mg, 14.574 mmol, 100% yield) as a pale orange solid, material was used without further purification.

¹H NMR (400 MHz, DMSO-d6) δ/ppm: 13.39 (br s, 1H), 7.93-7.92 (m, 1H), 7.58-7.56 (m, 1H), 2.63 (s, 3H).

Step D, 4-bromo-5-iodo-6-methyl-1-tetrahydropyran-2-yl-indazole. p-Toluenesulfonic acid monohydrate (510.12 mg, 2.96 mmol) was added to 4-bromo-5-iodo-6-methyl-1H-indazole (4991. mg, 14.8 mmol) and 3,4-Dihydro-2H-pyran (2.7 mL, 29.6 mmol) in THF (60 mL), and stirred at 50° C. overnight. The mixture reduced in volume in vacuo and diluted with EtOAc and water. The organics were washed with sat. NaHCO₃×2, and brine, dried with Na₂SO₄, filtered, and concentrated. The crude was purified by flash column chromatography eluting 0-15% EtOAc in petroleum ether. The desired fractions were concentrated and triturated with MeOH (10 mL). The solid was dried in vacuo to afford 4-bromo-5-iodo-6-methyl-1-tetrahydropyran-2-yl-indazole (3939 mg, 9.35 mmol, 63% yield) as a white solid.

UPLC-MS (ES⁺, Method 2): 2.21 min, m/z 421.0 [M+H]⁺.

Step E, 4-bromo-5-cyclopropyl-6-methyl-1-tetrahydropyran-2-yl-indazole. [1,1′-Bis(diphenylphosphino)ferrocene]Palladium(II) chloride dichloromethane complex (77.58 mg, 0.09 mmol) was added to 4-bromo-5-iodo-6-methyl-1-tetrahydropyran-2-yl-indazole (400 mg, 0.95 mmol), cesium carbonate (619.02 mg, 1.9 mmol), and cyclopropylboronic acid (89.8 mg, 1.04 mmol) in 1,4-dioxane (5.28 mL) and water (1.05 mL) degassed with N₂. The mixture was heated to 90° C. overnight. The reaction was concentrated in vacuo, and crude was purified by flash column chromatography eluting 5-30% EtOAc in petroleum ether. The desired fractions were concentrated to afford 4-bromo-5-cyclopropyl-6-methyl-1-tetrahydropyran-2-yl-indazole (214 mg, 0.6383 mmol, 67% yield) as a colourless oil.

UPLC-MS (ES⁺, Method 5): 2.36 min, m/z 335.0 [M+H]⁺.

Step F, 5-cyclopropyl-6-methyl-1-tetrahydropyran-2-yl-4-(4,4,5,5-tetramethyl-1,3,2-dioxaborolan-2-yl) indazole (A-22). [1,1′-Bis(diphenylphosphino)ferrocene]Palladium(II) chloride dichloromethane complex (52.13 mg, 0.06 mmol) was added to a solution of 4-bromo-5-cyclopropyl-6-methyl-1-tetrahydropyran-2-yl-indazole (214 mg, 0.64 mmol), bis(pinacolato)diboron (243.15 mg, 0.96 mmol), and potassium acetate (125.3 mg, 1.28 mmol) in 1,4-dioxane (4.2557 mL), degassed with N₂. The mixture was heated to 100° C. overnight. The reaction was concentrated in vacuo, and the crude was purified by flash column chromatography eluting 5-50% EtOAc in petroleum ether. The desired fractions were concentrated to afford 5-cyclopropyl-6-methyl-1-tetrahydropyran-2-yl-4-(4,4,5,5-tetramethyl-1,3,2-dioxaborolan-2-yl) indazole (57 mg, 0.149 mmol, 23% yield) as a colourless gum.

UPLC-MS (ES⁺, Method 5): 2.33 min, m/z 383.1 [M+H]⁺.

### Intermediate A-23, 5-ethyl-1,6-difluoro-4-(4,4,5,5-tetramethyl-1,3,2-dioxaborolan-2-yl)naphthalen-2-ol

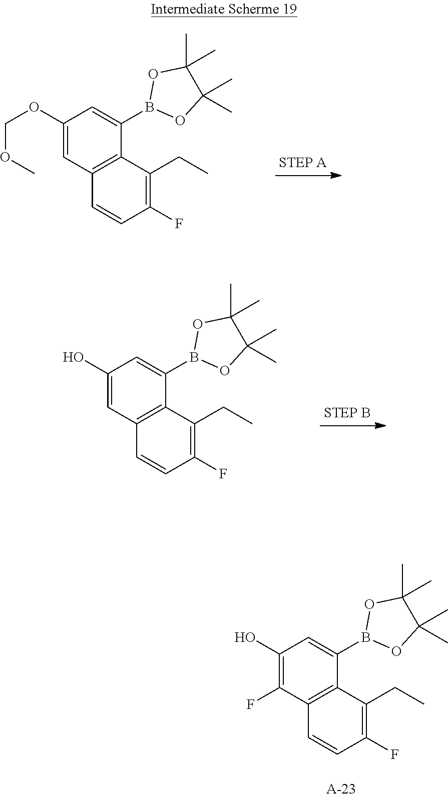

### Step A, 5-ethyl-6-fluoro-4-(4,4,5,5-tetramethyl-1,3,2-dioxaborolan-2-yl)naphthalen-2-ol

Hydrogen Chloride (4M in dioxane) (0.69 mL, 2.78 mmol) was added to a solution of 2-[8-ethyl-7-fluoro-3-(methoxymethoxy)-1-naphthyl]-4,4,5,5-tetramethyl-1,3,2-dioxaborolane (200 mg, 0.56 mmol) in DCM (2.78 mL) stirred at room temperature for 1 hr, then concentrated in vacuo. The crude was purified by flash column chromatography eluting 0-25% EtOAc in petroleum ether. The desired fractions were concentrated to afford 5-ethyl-6-fluoro-4-(4,4,5,5-tetramethyl-1,3,2-dioxaborolan-2-yl)naphthalen-2-ol (89 mg, 0.282 mmol, 51% yield) as a translucent pink oil.

UPLC-MS (ES⁺, Method 5): 2.14 min, m/z 317.1 [M+H]⁺.

### Step B, 5-ethyl-1,6-difluoro-4-(4,4,5,5-tetramethyl-1,3,2-dioxaborolan-2-yl)naphthalen-2-ol (A-23)

Selectfluor (251.43 mg, 0.71 mmol) was added to a solution of 5-ethyl-6-fluoro-4-(4,4,5,5-tetramethyl-1,3,2-dioxaborolan-2-yl)naphthalen-2-ol (187 mg, 0.59 mmol) in MeCN (3.94 mL), and stirred at room temperature for 3 hrs. The mixture was diluted with EtOAc, washed with water and brine, dried with Na₂SO₄, filtered and concentrated. The crude was purified by flash column chromatography eluting 0-25% EtOAc in petroleum ether. The desired fractions were concentrated to afford 5-ethyl-1,6-difluoro-4-(4,4,5,5-tetramethyl-1,3,2-dioxaborolan-2-yl)naphthalen-2-ol (87 mg, 0.2603 mmol, 44% yield) as a yellow solid.

¹H NMR (400 MHz, CDCl₃)/ppm: 7.84-7.77 (m, 1H), 7.43-7.36 (m, 1H), 7.29-7.21 (m, 1H), 5.59 (br s, 1H), 3.17-3.07 (m, 2H), 1.43 (s, 12H), 1.30-1.23 (t, J=7.5 Hz, 3H).

### Intermediate A-24, 4-bromo-6-methyl-5-(trifluoromethyl)-1H-indazole

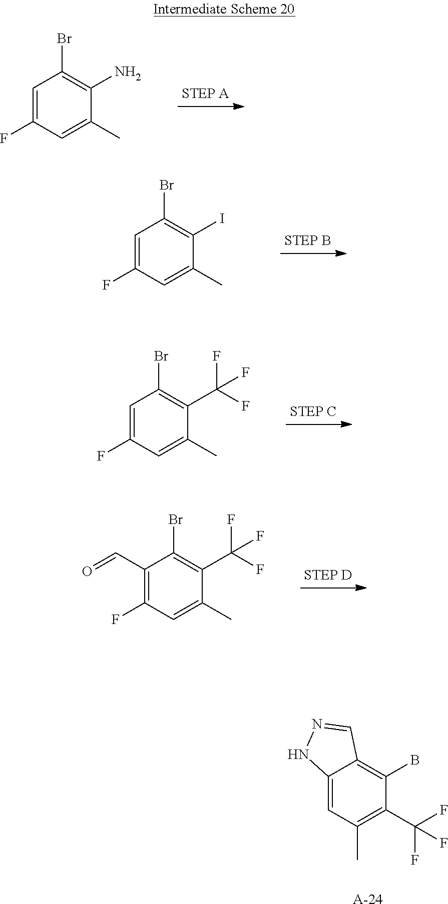

Step A, 1-bromo-5-fluoro-2-iodo-3-methyl-benzene. 2-bromo-4-fluoro-6-methylaniline (1 g, 4.9 mmol) was stirred at 70° C. with Hydrogen Chloride (6M in water) (10 mL, 60 mmol) for 30 minutes until completely dissolved. MeCN (4 mL) was added to aid solubility. The mixture was cooled to −5° C., and a solution of Sodium nitrite (405.74 mg, 5.88 mmol) in water (2.5 mL) was added. The mixture was stirred for 30 minutes, then potassium iodide (976.3 mg, 5.88 mmol) as a solution in water (4 mL) was added. The mixture was diluted with EtOAc (2×40 mL), and washed with brine (3×60 mL). The combined organics were dried with Na₂SO₄, filtered and concentrated to give a black solid. The crude was passed through a silica plug, eluting with 20% EtOAc in petroleum ether. The desired fractions were washed with sat. Na₂S₂O₃, dried and concentrated to afford 1-bromo-5-fluoro-2-iodo-3-methyl-benzene (908 mg, 2.8833 mmol, 59% yield) as a pale orange solid.

¹H NMR (400 MHz, CDCl₃) δ/ppm: 7.26-7.23 (m, 1H), 6.97-6.92 (m, 1H), 2.55 (s, 3H).

Step B, 1-bromo-5-fluoro-3-methyl-2-(trifluoromethyl)benzene. To a stirred solution of 1-bromo-5-fluoro-2-iodo-3-methyl-benzene (908. mg, 2.88 mmol) in DMF (14.42 mL) was added methyl-2,2-difluoro-2-(fluorosulfonyl)acetate (3.3 mL, 25.9 mmol) and methyl-2,2-difluoro-2-(fluorosulfonyl)acetate (3.3 mL, 25.95 mmol). The reaction was heated to 60° C. and stirred overnight. The reaction was allowed to cool to room temperature and diluted with 100 ml of water. The suspension was extracted with petroleum ether (2×100 mL). The combined organic extracts were washed with brine, dried via phase separator and evaporated to afford 1-bromo-5-fluoro-3-methyl-2-(trifluoromethyl)benzene (369 mg, 1.44 mmol, 50% yield) as a pale orange oil. Material was used without further purification.

UPLC-MS (ES⁺, Method 5): 2.23 min.

Step C, 2-bromo-6-fluoro-4-methyl-3-(trifluoromethyl)benzaldehyde. To a stirred solution of 1-bromo-5-fluoro-3-methyl-2-(trifluoromethyl)benzene (369 mg, 1.44 mmol) in THF (1.9 mL) under nitrogen at −65° C. was added Lithium diisopropylamide (0.8 mL, 1.59 mmol) (2 M in THF) dropwise. The reaction was stirred for 30 mins at −65° C. N,N-Dimethylformamide (0.12 mL, 1.58 mmol) was then added dropwise and the reaction continued to stir at this temperature. After 2 hours the reaction was warmed to 0° C. and quenched with 1 M HCl until the solution was pH 4. The aqueous mixture was extracted with EtOAc (3×). The combined organic extracts were washed with brine, dried via phase separator and evaporated to afford 2-bromo-6-fluoro-4-methyl-3-(trifluoromethyl)benzaldehyde (251 mg, 0.881 mmol, 61% yield) as a brown oil. Material was used without further purification.

UPLC-MS (ES⁺, Method 5): 2.00 min.

Step D, 4-bromo-6-methyl-5-(trifluoromethyl)-1H-indazole (A-24). To a stirred solution of 2-bromo-6-fluoro-4-methyl-3-(trifluoromethyl)benzaldehyde (250 mg, 0.88 mmol) in THF (2 mL) was added Hydrazine Hydrate (0.13 mL, 2.63 mmol). The reaction was stirred at 60° C. for 21 hrs. The reaction was partitioned between EtOAc and water and the layers separated. The aqueous layer was extracted with EtOAc. The combined organic extracts were washed with brine, dried via phase separator and evaporated to afford an orange oil. This material was purified via silica gel chromatography eluting 0-100% EtOAc in petroleum ether to afford 4-bromo-6-methyl-5-(trifluoromethyl)-1H-indazole (137 mg, 0.491 mmol, 56% yield) as a yellow solid (A-24).

UPLC-MS (ES⁺, Method 5): 1.93 min, m/z 280.8 [M+H]⁺.

Intermediate A-25, N,N,2-trimethyl-5-(4,4,5,5-tetramethyl-1,3,2-dioxaborolan-2-yl)pyrazole-3-carboxamide.

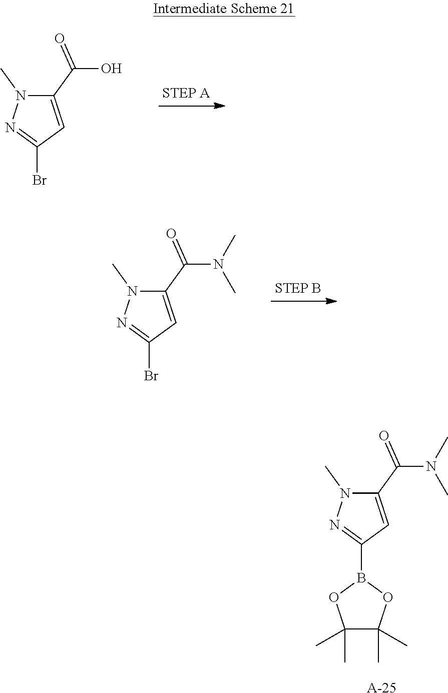

Step A, 3-bromo-N,N, 1-trimethyl-pyrazole-5-carboxamide. To a stirred solution of 3-bromo-1-methyl-pyrazole-5-carboxylic acid (500 mg, 2.44 mmol) in DMF (10 mL) at room temperature was added N,N-Diisopropylethylamine (1.27 mL, 7.32 mmol), N,N,N′,N′-tetramethyl-O-(benzotriazol-1-yl) uronium tetrafluoroborate (1370.41 mg, 4.27 mmol) and dimethylamine hydrochloride (298.3 mg, 3.66 mmol). The reaction was stirred overnight at room temperature. The reaction was partitioned between EtOAc and water and the layers separated. The aqueous was extracted with EtOAc (2×). The organic extracts were washed with water and brine, dried via phase separator and evaporated to afford 3-bromo-N,N,1-trimethyl-pyrazole-5-carboxamide (566 mg, 2.439 mmol, 100% yield) as an orange oil.

UPLC-MS (ES⁺, Method 5): 1.29 min, m/z 231.9, 233.9 [M+H]⁺.

¹H NMR (400 MHz, CDCl₃) δ/ppm: 6.34 (s, 1H), 3.94 (s, 3H), 3.11 (s, 7H).

Step B, N,N,2-trimethyl-5-(4,4,5,5-tetramethyl-1,3,2-dioxaborolan-2-yl)pyrazole-3-carboxamide (A-25). To a stirred solution of 3-bromo-N,N, 1-trimethyl-pyrazole-5-carboxamide (570 mg, 2.46 mmol) in 1,4-Dioxane (10 mL) was added 1,1′-Bis(diphenylphosphino)ferrocene (68.08 mg, 0.12 mmol), Bis(pinacolato)diboron (686.06 mg, 2.7 mmol), Potassium acetate (723.13 mg, 7.37 mmol) and [1,1′-Bis(diphenylphosphino)ferrocene]Palladium(II) chloride dichloromethane complex (100.29 mg, 0.12 mmol). The reaction was degassed by cycling vacuum and nitrogen three times, DMF (1 mL) was added to aid solubility and the reaction was stirred overnight at 100° C. The reaction was filtered through a pad of Celite, washing with EtOAc and the filtrate evaporated to afford N,N,2-trimethyl-5-(4,4,5,5-tetramethyl-1,3,2-dioxaborolan-2-yl)pyrazole-3-carboxamide (685 mg, 2.4539 mmol, 100% yield)

UPLC-MS (ES⁺, Method 5): 0.60 min, m/z 198.0 [M+H]⁺.

Intermediate A-26, tert-butyl N-[3-cyano-7-fluoro-4-(4,4,5,5-tetramethyl-1,3,2-dioxaborolan-2-yl)benzothiophen-2-yl]carbamate.

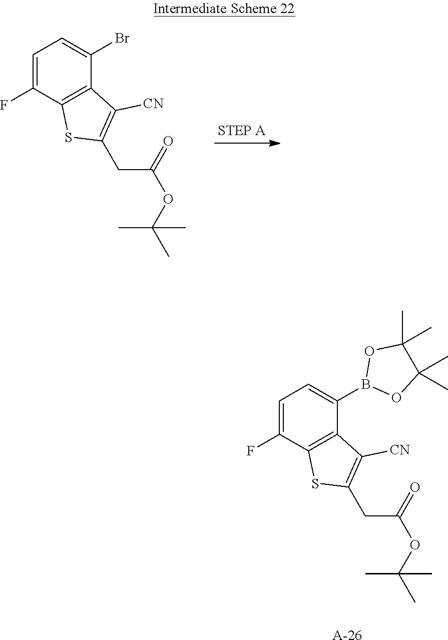 [SMILES: CC(C)(C)OC(=O)Cc1sc2c(F)ccc(Br)c2c1C#N ; CC(C)(C)OC(=O)Cc1sc2c(F)ccc(B3OC(C)(C)C(C)(C)O3)c2c1C#N ; "A-26"]

Step A, tert-butyl N-[3-cyano-7-fluoro-4-(4,4,5,5-tetramethyl-1,3,2-dioxaborolan-2-yl)benzothiophen-2-yl]carbamate (A-26). A mixture of [1,1′-bis(diphenylphosphino)ferrocene]dichloropalladium II (59.3 mg, 0.08 mmol) tert-Butyl (4-bromo-3-cyano-7-fluorobenzo[b]thiophen-2-yl)carbamate (300. mg, 0.81 mmol) potassium acetate (237.94 mg, 2.42 mmol) bis(pinacolato)diboron (410.44 mg, 1.62 mmol) in DMF (8.08 mL) in a sealed vial under N₂ atmosphere was stirred at 90° C. overnight. Then the mixture was extracted with EtOAc for three times and the organic phase was washed with water and brine. The organic phase was dried with sodium sulfate and filtered and concentrated in vacuum to give a brown oil, which was purified by flash chromatography eluting 0 to 5% EtOAc in Petroleum ether to give product tert-butyl N-[3-cyano-7-fluoro-4-(4,4,5,5-tetramethyl-1,3,2-dioxaborolan-2-yl)benzothiophen-2-yl]carbamate (408 mg, 0.6535 mmol, 81% yield) as a pale yellow oil.

UPLC-MS (ES⁺, Method 5): 2.42 min, m/z 419.0 [M+H]⁺.

Intermediate A-27, 2-(methoxymethoxymethyl)-5,6,7,8-tetrahydro-4H-pyrazolo[1,5-a][1,4]diazepine and intermediate A-36, 2-(methoxymethyl)-5,6,7,8-tetrahydro-4H-pyrazolo[1,5-a][1,4]diazepine.

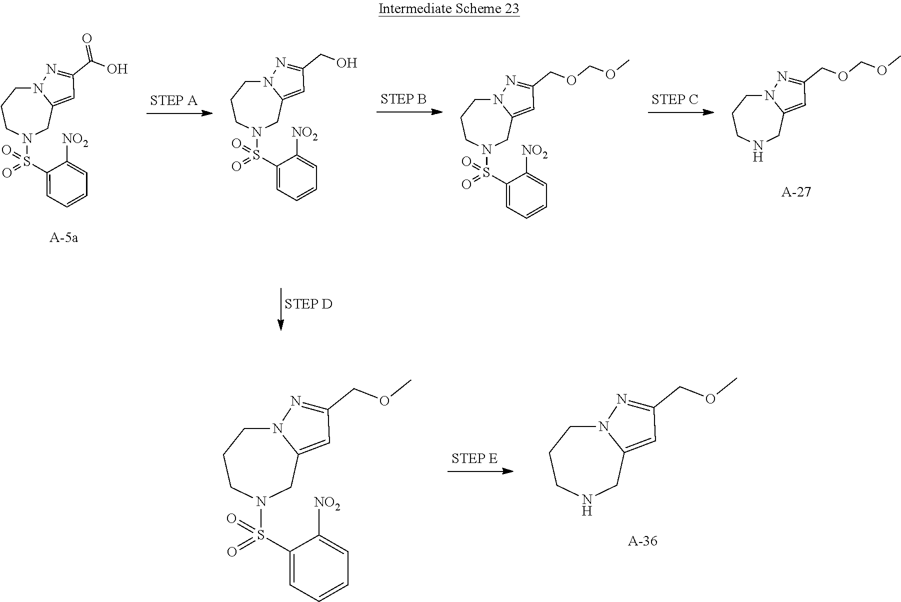

Step A, [5-(2-nitrophenyl) sulfonyl-4,6,7,8-tetrahydropyrazolo[1,5-a][1,4]diazepin-2-yl]methanol. Lithium borohydride (99.63 mg, 4.56 mmol) was added to ethyl 5-(2-nitrophenyl) sulfonyl-4,6,7,8-tetrahydropyrazolo[1,5-a][1,4]diazepine-2-carboxylate (1.5 g, 3.8 mmol) in dry THF (12.7 mL) at 0° C. and stirred at room temperature overnight. A further portion of lithium borohydride (41.51 mg, 1.9 mmol) was added and the reaction stirred for 3 hrs further at room temperature. The mixture was acidified to pH3 with 1N HCl and extracted with EtOAc (2×). The combined organic extracts were dried via phase separator and evaporated to afford an orange oil which was purified via silica gel chromatography eluting 0-10% MeOH in DCM to afford [5-(2-nitrophenyl) sulfonyl-4,6,7,8-tetrahydropyrazolo[1,5-a][1,4]diazepin-2-yl]methanol (448 mg, 1.27 mmol, 33% yield) as an orange solid.

¹H NMR (400 MHz, CDCl₃) δ/ppm: 7.98 (d, J=7.7 Hz, 1H), 7.77-7.60 (m, 3H), 6.26 (s, 1H), 5.32 (s, 1H), 4.63 (s, 2H), 4.57 (s, 2H), 4.44-4.32 (m, 2H), 3.78-3.64 (m, 2H), 2.10-1.94 (m, 2H).

UPLC-MS (ES⁺, Method 5): 1.39 min, m/z 353.0 [M+H]⁺.

Step B, 2-(methoxymethoxymethyl)-5-(2-nitrophenyl) sulfonyl-4,6,7,8-tetrahydropyrazolo[1,5-a][1,4]diazepine. Sodium hydride (60% dispersed in mineral oil) (26.7 mg, 0.67 mmol) was added to a stirred solution of [5-(2-nitrophenyl) sulfonyl-4,6,7,8-tetrahydropyrazolo[1,5-a][1,4]diazepin-2-yl]methanol (224. mg, 0.64 mmol) in THF (3.5 mL) at 0° C. The reaction was stirred for 30 mins at 0° C. Chloromethyl methyl ether (0.05 mL, 0.65 mmol) was added and the reaction was stirred at room temperature overnight. A further portion of Chloromethyl methyl ether (0.02 mL, 0.32 mmol) was added and the reaction stirred for 2 hours at room temperature. The reaction was partitioned between EtOAc and water and the layers separated. The aqueous layer was extracted with EtOAc (3×). The combined organic extracts were dried via phase separator and evaporated to afford a brown oil. This material was purified via silica gel chromatography eluting 0-80% EtOAc in petroleum ether to afford 2-(methoxymethoxymethyl)-5-(2-nitrophenyl) sulfonyl-4,6,7,8-tetrahydropyrazolo[1,5-a][1,4]diazepine (168 mg, 0.424 mmol, 67% yield) as a brown solid.

UPLC-MS (ES⁺, Method 5): 1.61 min, m/z 397.0 [M+H]⁺.

Step c, 2-(methoxymethoxymethyl)-5,6,7,8-tetrahydro-4H-pyrazolo[1,5-a][1,4]diazepine (A-27). To 2-(methoxymethoxymethyl)-5-(2-nitrophenyl) sulfonyl-4,6,7,8-tetrahydropyrazolo[1,5-a][1,4]diazepine (168. mg, 0.42 mmol) and Cesium Carbonate (276.16 mg, 0.85 mmol) in MeCN (2.5 mL) was added Thiophenol (0.09 mL, 0.85 mmol) and the reaction stirred at room temperature for 1 hr. The reaction mixture was filtered through Celite, washing with EtOAc. The filtrate was evaporated to afford a brown oil. This material was loaded on to an SCX-II Cartridge, eluting first with MeOH then with 1 M NH₃/MeOH. The ammoniacal fraction was evaporated to afford 2-(methoxymethoxymethyl)-5,6,7,8-tetrahydro-4H-pyrazolo[1,5-a][1,4]diazepine (57 mg, 0.2698 mmol, 64% yield) as a yellow gum. Material was used without further purification.

¹H NMR (400 MHz, CDCl₃) δ/ppm: 6.09 (s, 1H), 4.72 (s, 2H), 4.53 (s, 2H), 4.38-4.34 (m, 2H), 3.88 (s, 3H), 3.42-3.39 (m, 3H), 3.22-3.15 (m, 3H), 1.87-1.80 (m, 3H).

Step D, 2-(methoxymethyl)-5-(2-nitrophenyl) sulfonyl-4,6,7,8-tetrahydropyrazolo[1,5-a][1,4]diazepine. To a stirring solution of [5-(2-nitrophenyl) sulfonyl-4,6,7,8-tetrahydropyrazolo[1,5-a][1,4]diazepin-2-yl]methanol (220 mg, 0.62 mmol) in THF (6 mL) under an inert atmosphere, sodium hydride, (60% dispersed in mineral oil) (24.97 mg, 0.62 mmol) was added at 0° C. The reaction was left to stir for 30 minutes. Iodomethane (0.04 mL, 0.69 mmol) was added to the flask and the ice bath removed and left to stir for 2 h at 50° C. The reaction was quenched with water and extracted with EtOAc (30 mL). The organic layer was washed with water and brine (15 mL each), passed through a phase separator cartridge and concentrated. The residue was purified by flash chromatography (12 g column, gradient 0-100%, EtOAc in pet. ether) to afford 2-(methoxymethyl)-5-(2-nitrophenyl) sulfonyl-4,6,7,8-tetrahydropyrazolo[1,5-a][1,4]diazepine (144 mg, 0.393 mmol, 34.11% yield) as an orange solid.

UPLC-MS (ES⁺, Method 5): 1.56 min, m/z 367.1 [M+H]⁺

¹H NMR (400 MHz, CDCl₃) δ 7.96-7.92 (m, 1H), 7.72-7.58 (m, 3H), 6.24 (s, 1H), 4.56 (s, 2H), 4.42-4.33 (m, 4H), 3.72-3.65 (m, 2H), 3.38 (s, 3H), 2.06-1.97 (m, 2H).

Step E, 2-(methoxymethyl)-5,6,7,8-tetrahydro-4H-pyrazolo[1,5-a][1,4]diazepine (A-36). To 2-(methoxymethyl)-5-(2-nitrophenyl) sulfonyl-4,6,7,8-tetrahydropyrazolo[1,5-a][1,4]diazepine (144 mg, 0.39 mmol) and cesium carbonate (256 mg, 0.79 mmol) in MeCN (2.5 mL) was added thiophenol (0.08 mL, 0.79 mmol) and the reaction stirred at room temperature for 2 hours. The reaction mixture was filtered through Celite, washing with EtOAc. The filtrate was evaporated to afford a brown oil. This material was loaded on to an SCX-II cartridge, eluting first with MeOH then with 1 M NH₃/MeOH. The ammoniacal fraction was evaporated to afford 2-(methoxymethyl)-5,6,7,8-tetrahydro-4H-pyrazolo[1,5-a][1,4]diazepine (63 mg, 0.3476 mmol, 88% yield) as an orange oil.

UPLC-MS (ES⁺, Method 5): 0.33 min, m/z 182.1 [M+H]⁺

¹H NMR (400 MHz, DMSO-d6) δ 6.01 (s, 1H), 4.32 (s, 2H), 4.31-4.26 (m, 2H), 3.81 (s, 2H), 3.32 (s, 3H), 3.15-3.09 (m, 2H), 1.91-1.82 (m, 1H), 1.80-1.72 (m, 2H).

### Intermediate A-28, N,N-dimethyl-1-(5,6,7,8-tetrahydro-4H-pyrazolo[1,5-a][1,4]diazepin-2-yl) methanamine

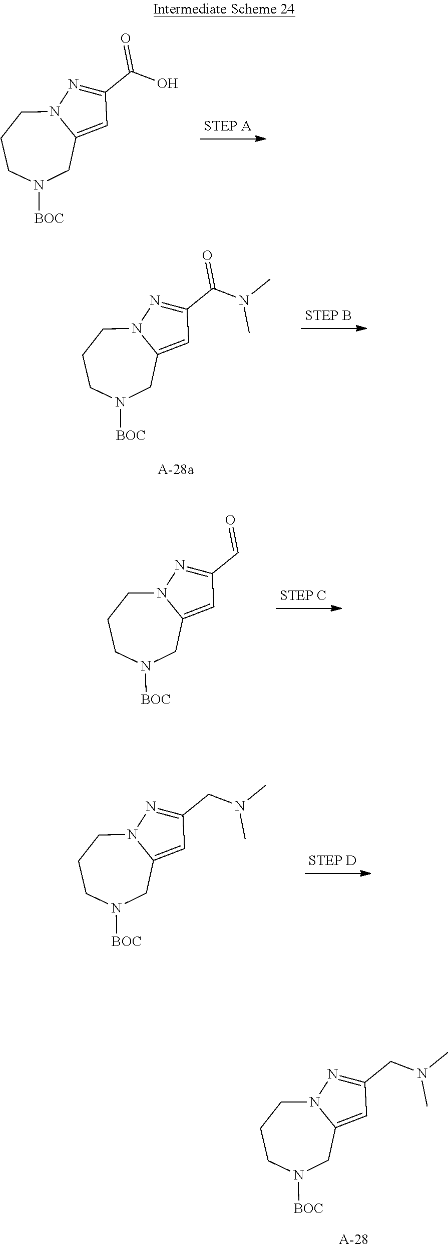 [SMILES: CC(C)(C)OC(=O)N1CCCn2nc(C(=O)O)cc2C1 ; CN(C)C(=O)c1cc2n(n1)CCCN(C(=O)OC(C)(C)C)C2 ; CC(C)(C)OC(=O)N1CCCn2nc(C=O)cc2C1 ; CN(C)Cc1cc2n(n1)CCCN(C(=O)OC(C)(C)C)C2 ; CN(C)Cc1cc2n(n1)CCCN(C(=O)OC(C)(C)C)C2 ; "A-28"]

Step A, tert-butyl 2-(dimethylcarbamoyl)-4,6,7,8-tetrahydropyrazolo[1,5-a][1,4]diazepine-5-carboxylate. Propylphosphonic anhydride (9.52 mL, 16. mmol) and N,N-diisopropylethylamine (3.72 mL, 21.3 mmol) were added to a flask containing 5-(tert-butoxycarbonyl)-5,6,7,8-tetrahydro-4H-pyrazolo[1,5-a][1,4]diazepine-2-carboxylic acid (3000 mg, 10.7 mmol) and dimethylamine hydrochloride (1304.36 mg, 16.0 mmol) in THF (100 mL). The reaction mixture was heated to 65° C. and left to stir overnight. Added further dimethylamine hydrochloride (434.79 mg, 5.33 mmol) and N, N-Diisopropylethylamine (1.86 mL, 10.66 mmol), followed by propylphosphonic anhydride (6.35 mL, 10.66 mmol). The reaction mixture was heated to 65° C. and left to stir overnight. The reaction mixture was cooled to room temperature and diluted with 500 ml water. The aqueous layer was extracted with EtOAc (3×150 mL). The combined organics were washed with brine (200 mL), passed through a phase separator and reduced in vacuo to afford tert-butyl 2-(dimethylcarbamoyl)-4,6,7,8-tetrahydropyrazolo[1,5-a][1,4]diazepine-5-carboxylate (3.188 g, 10.338 mmol, 97% yield) as a white solid.

UPLC-MS (ES⁺, Method 5): 1.50 min, m/z 309.1 [M+H]⁺.

¹H NMR (400 MHz, DMSO-d6) δ/ppm: 6.43 (s, 1H), 4.47 (s, 2H), 4.42 (m, 2H), 3.65 (s, 2H), 3.24 (s, 3H), 2.94 (s, 3H), 1.76 (s, 2H), 1.33 (s, 9H) ppm.

Step B, tert-butyl 2-formyl-4,6,7,8-tetrahydropyrazolo[1,5-a][1,4]diazepine-5-carboxylate. To a stirred solution of tert-butyl 2-(dimethylcarbamoyl)-4,6,7,8-tetrahydropyrazolo[1,5-a][1,4]diazepine-5-carboxylate (500. mg, 1.62 mmol) in THF (10 mL) at 0° C. was added Lithium aluminium hydride (1.35 mL, 3.24 mmol) (2.4 M in THF) dropwise. The reaction was allowed to warm to RT and stirred for 30 mins. Glauber's salt was added cautiously until no more effervescence occurred, then the reaction mixture was filtered. The filtrate was evaporated to afford tert-butyl 2-formyl-4,6,7,8-tetrahydropyrazolo[1,5-a][1,4]diazepine-5-carboxylate (430 mg, 1.6207 mmol, 100% yield) as a colourless oil. Used without purification in reductive amination step.

UPLC-MS (ES⁺, Method 5): 1.60 min, m/z 266.0 [M+H]⁺.

Step C, tert-butyl 2-[(dimethylamino)methyl]-4,6,7,8-tetrahydropyrazolo[1,5-a][1,4]diazepine-5-carboxylate (A-28). To a stirred solution of tert-butyl 2-formyl-4,6,7,8-tetrahydropyrazolo[1,5-a][1,4]diazepine-5-carboxylate (430. mg, 1.62 mmol) in DCM (10 mL) was added dimethylamine (2.43 mL, 4.86 mmol) (2 M in THF) and acetic acid (Glacial) (0.14 mL, 2.43 mmol). The reaction was stirred for 30 mins at room temperature. Sodium triacetoxyborohydride (1030.5 mg, 4.86 mmol) was added and the reaction was stirred for 1 hr. The reaction was quenched cautiously with saturated aqueous NaHCO₃ then extracted with DCM (2×). The combined organic layers were dried via phase separator and evaporated to afford a yellow oil which was purified via silica gel chromatography eluting 0-30% MeOH in DCM to afford tert-butyl 2-[(dimethylamino)methyl]-4,6,7,8-tetrahydropyrazolo[1,5-a][1,4]diazepine-5-carboxylate (184 mg, 0.625 mmol, 39% yield) as a colourless gum.

UPLC-MS (ES⁺, Method 5): 1.19 min, m/z 295.1 [M+H]⁺.

¹H NMR (400 MHz, CDCl₃) δ/ppm: 6.21 (s, 1H), 4.52-4.30 (m, 4H), 3.74-3.64 (m, 2H), 3.64-3.55 (m, 2H), 2.38 (s, 6H), 1.96-1.86 (m, 2H), 1.41 (s, 9H).

Step D, N,N-dimethyl-1-(5,6,7,8-tetrahydro-4H-pyrazolo[1,5-a][1,4]diazepin-2-yl) methanamine (A-28). To a stirred solution of tert-butyl 2-[(dimethylamino)methyl]-4,6,7,8-tetrahydropyrazolo[1,5-a][1,4]diazepine-5-carboxylate (184. mg, 0.63 mmol) in DCM (10 mL) at room temperature was added Trifluoroacetic acid (1.2 mL, 15.63 mmol). The reaction was stirred for 1 hr then loaded directly on to an SCX-II Cartridge, eluting first with MeOH and then with 1 M NH₃/MeOH. The ammoniacal fraction was evaporated to afford N, N-dimethyl-1-(5,6,7,8-tetrahydro-4H-pyrazolo[1,5-a][1,4]diazepin-2-yl) methanamine (102 mg, 0.525 mmol, 84% yield) as a colourless oil.

¹H NMR (400 MHz, CDCl₃) δ/ppm: 6.04 (s, 1H), 4.39-4.31 (m, 2H), 3.87 (s, 2H), 3.43 (s, 2H), 3.23-3.14 (m, 2H), 2.28 (s, 6H), 1.86-1.78 (m, 2H).

### Intermediate A-29, 3-fluoro-N,N-dimethyl-5,6,7,8-tetrahydro-4H-pyrazolo[1,5-a][1,4]diazepine-2-carboxamide

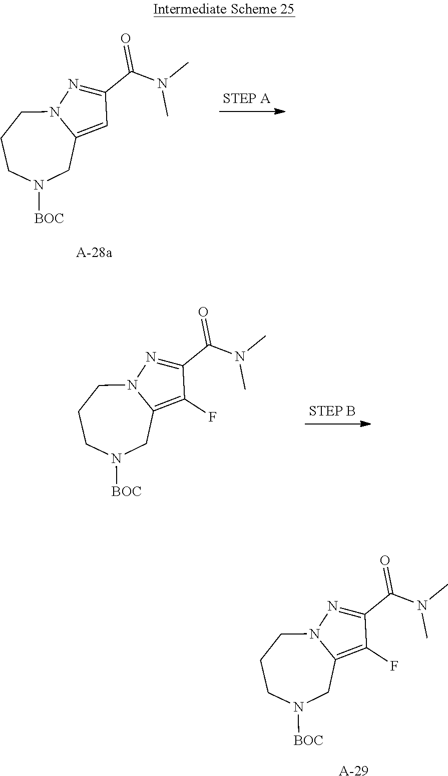 [SMILES: CN(C)C(=O)c1cc2n(n1)CCCN(C(=O)OC(C)(C)C)C2 ; CN(C)C(=O)c1nn2c(c1F)CN(C(=O)OC(C)(C)C)CCC2 ; CN(C)C(=O)c1nn2c(c1F)CN(C(=O)OC(C)(C)C)CCC2 ; "A-29"]

Step A, tert-butyl 2-(dimethylcarbamoyl)-3-fluoro-4,6,7,8-tetrahydropyrazolo[1,5-a][1,4]diazepine-5-carboxylate. N-chloromethyl-N′-fluorotriethylenediammonium bis(tetrafluoroborate)-(selectfluor) (2297.6 mg, 6.49 mmol) was added to a solution of tert-butyl 2-(dimethylcarbamoyl)-4,6,7,8-tetrahydropyrazolo[1,5-a][1,4]diazepine-5-carboxylate (1000. mg, 3.24 mmol) in MeCN (16 mL) and the suspension was stirred at 50° C. The reaction mixture was stirred overnight at room temperature. The mixture was filtered and the filter cake was washed with a minimal amount of MeCN. The filtrate was concentrated in vacuo. The residue taken up in DCM (50 mL) washed with H₂O (50 mL). The aqueous layer was extracted with DCM. The combined organics were passed through a phase separator and reduced in vacuo. The material was purified by reverse phase chromatography (eluting 0-100% MeCN in H₂O (+0.1% FA)). The relevant fractions were united and reduced in vacuo, then co-distilled with MeOH to afford tert-butyl 2-(dimethylcarbamoyl)-3-fluoro-4,6,7,8-tetrahydropyrazolo[1,5-a][1,4]diazepine-5-carboxylate (328 mg, 1.01 mmol, 31% yield) as a red oil.

¹H NMR (400 MHz, CDCl₃) δ/ppm: 4.58-4.31 (m, 4H), 3.77-3.67 (m, 2H), 3.18 (s, 3H), 3.09 (s, 3H), 1.98-1.88 (m, 2H), 1.43 (s, 9H).

Step B, 3-fluoro-N,N-dimethyl-5,6,7,8-tetrahydro-4H-pyrazolo[1,5-a][1,4]diazepine-2-carboxamide (A-29). Trifluoroacetic acid (0.77 mL, 10.05 mmol) was added to a stirring solution of tert-butyl 2-(dimethylcarbamoyl)-3-fluoro-4,6,7,8-tetrahydropyrazolo[1,5-a][1,4]diazepine-5-carboxylate (328. mg, 1. mmol) in DCM (5 mL). The reaction mixture was left to stir at room temperature for 2 hours. The reaction mixture was concentrated in vacuo and purified by SCX-2 column (5 g, washing with MeOH (×3) and eluting in 1N NH₃/MeOH (×3)). Ammoniacal fraction was evaporated in vacuo to afford 3-fluoro-N, N-dimethyl-5,6,7,8-tetrahydro-4H-pyrazolo[1,5-a][1,4]diazepine-2-carboxamide (200 mg, 0.8839 mmol, 88% yield) as a red oil.

UPLC-MS (ES⁺, Method 5): 0.31 min, m/z 227.0 [M+H]⁺.

### Intermediate A-35

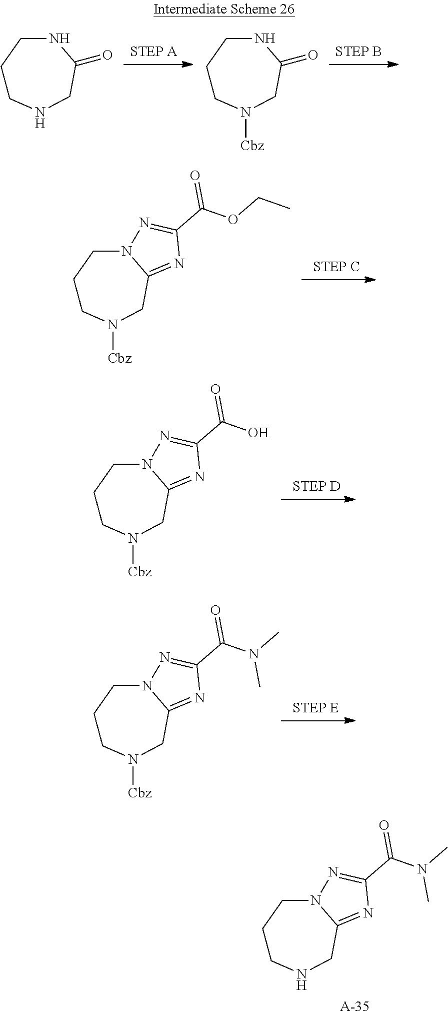

Step A, benzyl 3-oxo-1,4-diazepane-1-carboxylate. To a solution of 1,4-diazepan-2-one (1.09 g, 9.55 mmol) and triethylamine (2.7 mL, 19.37 mmol) in DCM (24 mL) at 0° C., under N₂, was added benzyl chloroformate (1.5 mL, 10.51 mmol) dropwise. The mixture was stirred at 20° C. overnight. The mixture was diluted with DCM (60 mL) and washed with water (20 mL) and brine (20 mL). The organic layer was passed through a phase separator cartridge and concentrated. The residue was purified by flash chromatography (gradient 0-20% methanol in DCM) to afford benzyl 3-oxo-1,4-diazepane-1-carboxylate (2.054 g, 8.2729 mmol, 86% yield) as a white solid.

UPLC-MS (ES+, Method 5): 1.44 min, m/z 249.0 [M+H]⁺.

¹H NMR (400 MHz, CDCl₃) δ/ppm: 7.41-7.28 (5H, m), 6.16-5.90 (1H, m), 5.17 (2H, s), 4.23-4.11 (2H, m), 3.70-3.61 (2H, m), 3.34-3.23 (2H, m), 1.95-1.84 (2H, m).

Step B, O8-benzyl O2-ethyl 5,6,7,9-tetrahydro-[1,2,4]triazolo[1,5-a][1,4]diazepine-2,8-dicarboxylate. To a solution of benzyl 3-oxo-1,4-diazepane-1-carboxylate (1.05 g, 4.23 mmol) in anhydrous DMF (40 mL) at 0° C., under N₂, was added sodium hydride, (60% dispersed in mineral oil) (254. mg, 6.35 mmol) and the mixture was stirred for 45 minutes. O-(diphenylphosphinyl)-hydroxylamine (1.48 g, 6.35 mmol) was added and the white suspension was stirred at 200C for 3.5 hours. The mixture was filtered, and the filtrate concentrated. The residue was dissolved in ethanol (15 mL) and ethyl 2-ethoxy-2-iminoacetate (1.5 mL, 10.77 mmol) was added. The mixture was heated at 90° C. for 18 hours. After cooling to room temperature, the mixture was concentrated, and the residue was purified twice by flash chromatography (gradient 0-8% methanol in DCM then 0-100% EtOAc in petroleum ether) to afford 08-benzyl O2-ethyl 5,6,7,9-tetrahydro-[1,2,4]triazolo[1,5-a][1,4]diazepine-2,8-dicarboxylate (643 mg, 1.8672 mmol, 44% yield) as a light yellow gum.

UPLC-MS (ES+, Method 5): 1.65 min, m/z 345.1 [M+H]⁺.

¹H NMR (400 MHz, DMSO-d6) δ/ppm: 7.40-7.24 (5H, m), 5.06 (2H, s), 4.81-4.72 (2H, m), 4.49-4.44 (2H, m), 4.30 (2H, q, J=7.1 Hz), 3.76 (2H, br s), 1.95 (2H, br s), 1.29 (3H, t, J=7.1 Hz).

Step C, 8-benzyloxycarbonyl-5,6,7,9-tetrahydro-[1,2,4]triazolo[1,5-a][1,4]diazepine-2-carboxylic acid. To a solution of 08-benzyl 02-ethyl 5,6,7,9-tetrahydro-[1,2,4]triazolo[1,5-a][1,4]diazepine-2,8-dicarboxylate (1.21 g, 3.5 mmol) in THF (17 mL) was added lithium hydroxide hydrate (1:1:1) (294.3 mg, 7.01 mmol) in water (3.5 mL). The mixture was stirred at 20° C. for 90 minutes and concentrated. The residue was diluted with water (10 mL) and acidified to pH 2-3 with 1N aq. HCl. The mixture was extracted with DCM (5×10 mL). The combined organic layers were passed through a phase separator cartridge and concentrated to afford 8-benzyloxycarbonyl-5,6,7,9-tetrahydro-[1,2,4]triazolo[1,5-a][1,4]diazepine-2-carboxylic acid (1.034 g, 3.2688 mmol, 93% yield) as a white solid.

UPLC-MS (ES+, Method 5): 1.41 min, m/z 317.1 [M+H]⁺.

Step D, benzyl 2-(dimethylcarbamoyl)-5,6,7,9-tetrahydro-[1,2,4]triazolo[1,5-a][1,4]diazepine-8-carboxylate. To a solution of 8-benzyloxycarbonyl-5,6,7,9-tetrahydro-[1,2,4]triazolo[1,5-a][1,4]diazepine-2-carboxylic acid (1.03 g, 3.27 mmol) and dimethylamine hydrochloride (400. mg, 4.91 mmol) in THF (22 mL) were successively added propylphosphonic anhydride (3.9 mL, 6.55 mmol) and N,N-diisopropylethylamine (2.6 mL, 14.93 mmol). The mixture was stirred at 20° C. overnight. The mixture was concentrated, and the residue dissolved in EtOAc (60 mL). The organic layer was washed with water (2×20 mL) and brine (20 mL), passed through a phase separator cartridge and concentrated to afford benzyl 2-(dimethylcarbamoyl)-5,6,7,9-tetrahydro-[1,2,4]triazolo[1,5-a][1,4]diazepine-8-carboxylate (880 mg, 2.5627 mmol, 78% yield) as a very thick yellow oil.

UPLC-MS (ES+, Method 5): 1.51 min, m/z 344.1 [M+H]⁺.

¹H NMR (400 MHz, DMSO-d6) δ/ppm: 7.39-7.23 (5H, m), 5.06 (2H, s), 4.79-4.71 (2H, m), 4.44-4.37 (2H, m), 3.81-3.73 (2H, m), 3.08-3.00 (3H, m), 2.96 (3H, s), 2.01-1.90 (2H, m).

Step E, N,N-dimethyl-6,7,8,9-tetrahydro-5H-[1,2,4]triazolo[1,5-a][1,4]diazepine-2-carboxamide (A-35). A flask containing a solution of benzyl 2-(dimethylcarbamoyl)-5,6,7,9-tetrahydro-[1,2,4]triazolo[1,5-a][1,4]diazepine-8-carboxylate (880. mg, 2.56 mmol) in EtOAc (25 mL) was evacuated/backfilled with N₂ then Palladium, 10 wt % on carbon, 55-65% wet (546. mg, 0.26 mmol) was added. The flask was then evacuated/backfilled with hydrogen and the mixture was stirred at 20° C., under a hydrogen atmosphere, overnight. The flask was evacuated/backfilled with N₂. The mixture was concentrated, and the filter cake washed with EtOAc. The filtrate was concentrated to afford N, N-dimethyl-6,7,8,9-tetrahydro-5H-[1,2,4]triazolo[1,5-a][1,4]diazepine-2-carboxamide (464 mg, 2.2174 mmol, 87% yield) as a white solid.

UPLC-MS (ES+, Method 5): 0.26 min, m/z 210.1 [M+H]⁺.

¹H NMR (400 MHz, DMSO-d6) δ/ppm: 4.35-4.31 (2H, m), 3.91 (2H, s), 3.06 (3H, s), 3.06-3.02 (2H, m), 2.95 (3H, s), 2.60 (1H, br s), 1.82-1.75 (2H, m).

### Intermediate A-30, 2-(3-chloro-8-ethyl-7-fluoro-1-naphthyl)-4,4,5,5-tetramethyl-1,3,2-dioxaborolane

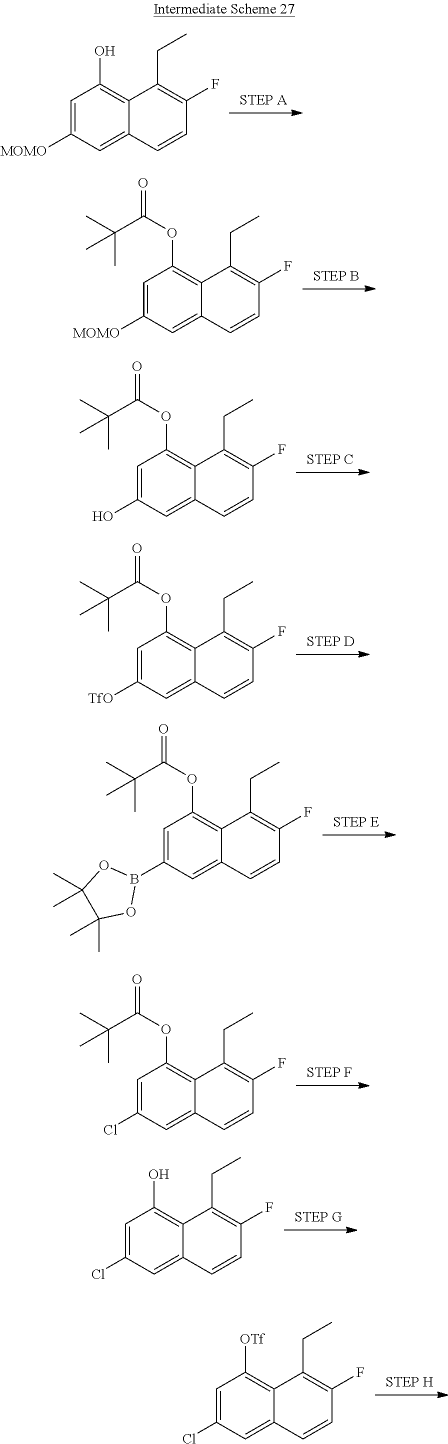

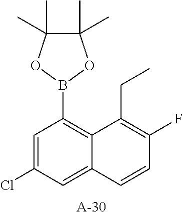

Step A, [8-ethyl-7-fluoro-3-(methoxymethoxy)-1-naphthyl] 2,2-dimethylpropanoate. To a solution of 8-ethyl-7-fluoro-3-(methoxymethoxy) naphthalen-1-ol (1000 mg, 4 mmol) and N,N-Diisopropylethylamine (0.84 mL, 4.79 mmol) in DCM (7.99 mL) was added trimethyl acetyl chloride (0.59 mL, 4.79 mmol) at 0° C. Allowed to stir at room temperature for 2 hrs. Extra N,N-Diisopropylethylamine (0.21 mL, 1.2 mmol) and trimethyl acetyl chloride (0.15 mL, 1.2 mmol) were added. Reaction mixture stirred at room temperature overnight. Partitioned between a layer of DCM (20 mL) and water (20 mL). Organic layer separated and aqueous layer extracted with DCM (2×20 mL). Organic layers combined, filtered over hydrophobic frit and concentrated under reduced pressure to afford [8-ethyl-7-fluoro-3-(methoxymethoxy)-1-naphthyl] 2,2-dimethylpropanoate (1336.1 mg, 4 mmol, 100% yield) as a brown oil.

UPLC-MS (ES⁺, Method 2): 2.24 min, m/z 335.2 [M+H]⁺.

Step B, (8-ethyl-7-fluoro-3-hydroxy-1-naphthyl) 2,2-dimethylpropanoate. Hydrogen Chloride (4.99 mL, 19.98 mmol) (4N in dioxane) was added to a solution of [8-ethyl-7-fluoro-3-(methoxymethoxy)-1-naphthyl] 2,2-dimethylpropanoate (1336.1 mg, 4 mmol) in DCM (7.99 mL). Afterwards, allowed to stir at room temperature for 1 hr. Concentrated under reduced pressure. Material was purified by flash column chromatography eluting in 0-70% EtOAc in petroleum ether affording (8-ethyl-7-fluoro-3-hydroxy-1-naphthyl) 2,2-dimethylpropanoate (955.2 mg, 3.29 mmol, 83% yield) as a brown oil.

UPLC-MS (ES⁺, Method 5): 2.47 min, m/z 291.0 [M+H]⁺.

Step C, [8-ethyl-7-fluoro-3-(trifluoromethylsulfonyloxy)-1-naphthyl] 2,2-dimethylpropanoate. At 0° C., Trifluoromethanesulfonic anhydride (0.13 mL, 0.76 mmol) was added to a solution of N, N-Diisopropylethylamine (0.3 mL, 1.72 mmol) and (8-ethyl-7-fluoro-3-hydroxy-1-naphthyl) 2,2-dimethylpropanoate (200. mg, 0.69 mmol) in DCM (3.44 mL). Afterwards, allowed to stir at that temperature for 15 min. Reaction mixture was diluted with ice-water (20 mL) and extracted with EtOAc (3×20 mL). Organic layers were combined, washed with a saturated solution of brine (10 mL), dried over sodium sulfate, filtered and concentrated under reduced pressure. Material was purified by flash column chromatography (eluting 0-50% EtOAc in petroleum ether) affording [8-ethyl-7-fluoro-3-(trifluoromethylsulfonyloxy)-1-naphthyl] 2,2-dimethylpropanoate (225 mg, 0.5327 mmol, 78% yield) as an off-white solid.

UPLC-MS (ES⁺, Method 5): 2.51 min, m/z 421.0 [M+H]⁺.

¹H NMR (400 MHz, CDCl₃) δ/ppm: 7.74 (dd, J=9.0, 5.6 Hz, 1H), 7.64 (d, J=2.6 Hz, 1H), 7.36 (t, J=9.2 Hz, 1H), 6.94 (d, J=2.5 Hz, 1H), 3.21-3.13 (m, 2H), 1.47 (s, 9H), 1.28-1.21 (m, 3H).

Step D, [8-ethyl-7-fluoro-3-(4,4,5,5-tetramethyl-1,3,2-dioxaborolan-2-yl)-1-naphthyl] 2,2-dimethylpropanoate. A mixture of Bis(pinacolato)diboron (201.96 mg, 0.8 mmol), [8-ethyl-7-fluoro-3-(trifluoromethylsulfonyloxy)-1-naphthyl] 2,2-dimethylpropanoate (225. mg, 0.53 mmol) and Potassium acetate (182.99 mg, 1.86 mmol) in 1,4-Dioxane (2.6634 mL) was degassed with N₂ for 10 minutes then [1,1′-Bis(diphenylphosphino)ferrocene]Palladium(II) chloride dichloromethane complex (43.5 mg, 0.05 mmol) was added and the reaction mixture was heated at 100° C. for 1 hr. The reaction mixture was cooled down to room temperature. Diluted with EtOAc (10 mL), filtered over a celite plug, plug washed with EtOAc (2×10 mL), filtrate collected and concentrated under reduced pressure. Material was purified by flash column chromatography eluting 0-50% EtOAc in petroleum ether affording [8-ethyl-7-fluoro-3-(4,4,5,5-tetramethyl-1,3,2-dioxaborolan-2-yl)-1-naphthyl] 2,2-dimethylpropanoate (206 mg, 0.5146 mmol, 97% yield) as an off-white solid.

UPLC-MS (ES⁺, Method 5): 2.64 min, m/z 401.1 [M+H]⁺.

Step E, (3-chloro-8-ethyl-7-fluoro-1-naphthyl) 2,2-dimethylpropanoate. To a microwave vial, copper (ii) chloride (502.1 mg, 3.73 mmol) was added to [8-ethyl-7-fluoro-3-(4,4,5,5-tetramethyl-1,3,2-dioxaborolan-2-yl)-1-naphthyl] 2,2-dimethylpropanoate (498.3 mg, 1.24 mmol) in a mixture of MeCN (4.67 mL) and Water (1.56 mL). Afterwards, the vial was sealed and allowed to stir at 90° C. overnight. Reaction mixture allowed to cool back down to room temperature. Partitioned between a layer of EtOAc (20 mL) and water (20 mL). Organic layer separated and aqueous layer extracted with EtOAc (2×10 mL). Organic layers combined, washed with a saturated solution of brine (10 mL), dried over sodium sulfate, filtered and concentrated under reduced pressure. Material was purified by flash column chromatography eluting 0-30% EtOAc in petroleum ether affording (3-chloro-8-ethyl-7-fluoro-1-naphthyl) 2,2-dimethylpropanoate (260.8 mg, 0.8446 mmol, 67% yield) as a clear oil.

UPLC-MS (ES−, Method 5): 2.51 min, m/z 307.0 [M−H].

Step F, 3-chloro-8-ethyl-7-fluoro-naphthalen-1-ol. Potassium hydroxide (210.68 mg, 3.76 mmol) was added to a solution of (3-chloro-8-ethyl-7-fluoro-1-naphthyl) 2,2-dimethylpropanoate (463.8 mg, 1.5 mmol) in Methanol (7.5 mL). Afterwards, allowed to stir at room temperature for 30 min. Acidified to ~pH2 with 2M HCl (aq). Reaction mixture partitioned between a layer of ethyl acetate (20 mL) and water (20 mL). The organic layer was separated. The aqueous layer was extracted with EtOAc (20 mL), organic extracts combined, washed with a saturated solution of brine (20 mL), dried over sodium sulfate, filtered and concentrated under reduced pressure. Material was purified by flash column chromatography eluting 0-50% EtOAc in petroleum ether affording 3-chloro-8-ethyl-7-fluoro-naphthalen-1-ol (257 mg, 1.41 mmol, 76% yield) as a yellow solid.

UPLC-MS (ES⁺, Method 5): 2.14 min, m/z 223.0 [M−H].

Step G, (3-chloro-8-ethyl-7-fluoro-1-naphthyl)trifluoromethanesulfonate. At 0

° C., Trifluoromethanesulfonic anhydride (0.21 mL, 1.26 mmol) was added to a solution of N,N-Diisopropylethylamine (0.5 mL, 2.86 mmol) and 3-chloro-8-ethyl-7-fluoro-naphthalen-1-ol (257.3 mg, 1.15 mmol) in DCM (5.73 mL). Afterwards, allowed to stir at that temperature for 15 min. Reaction mixture was diluted with ice-water (20 mL) and extracted with EtOAc (3×20 mL). Organic layers were combined, washed with a saturated solution of brine (10 mL), dried over sodium sulfate, filtered and concentrated under reduced pressure. Material was purified by flash column chromatography eluting 0-30% EtOAc in petroleum ether affording (3-chloro-8-ethyl-7-fluoro-1-naphthyl)trifluoromethanesulfonate (253.2 mg, 0.710 mmol, 62% yield) as an off-white solid.

UPLC-MS (ES⁺, Method 5): 2.51 min, m/z 354.9 [M−2H]⁻.

Step H, 2-(3-chloro-8-ethyl-7-fluoro-1-naphthyl)-4,4,5,5-tetramethyl-1,3,2-dioxaborolane (A-30). A mixture of Bis(pinacolato)diboron (269.11 mg, 1.06 mmol), (3-chloro-8-ethyl-7-fluoro-1-naphthyl)trifluoromethanesulfonate (253.2 mg, 0.71 mmol) and potassium acetate (139.32 mg, 1.42 mmol) in 1,4-Dioxane (3.55 mL) was degassed with N2 for 2 min then [1,1′-Bis(diphenylphosphino)ferrocene]Palladium(II) chloride dichloromethane complex (57.97 mg, 0.07 mmol) was added and the reaction mixture was heated at 80° C. for 1 hr. Extra [1,1′-Bis(diphenylphosphino)ferrocene]Palladium(II) chloride dichloromethane complex (57.97 mg, 0.07 mmol) was added. Reaction mixture allowed to stir at 80° C. overnight. The reaction mixture was cooled down to room temperature. Diluted with EtOAc (10 mL), filtered over a celite plug, plug washed with EtOAc (2×10 mL), filtrate collected and concentrated under reduced pressure. Purification by flash column chromatography on silica gel eluting with 0-30% ethyl acetate in petrol affording 2-(3-chloro-8-ethyl-7-fluoro-1-naphthyl)-4,4,5,5-tetramethyl-1,3,2-dioxaborolane (77.8 mg, 0.2325 mmol, 33% yield) as an off-white solid.

¹H NMR (400 MHz, CDCl₃) δ/ppm: 7.81 (d, J=2.3 Hz, 1H), 7.59 (dd, J=9.0, 5.8 Hz, 1H), 7.55 (d, J=2.2 Hz, 1H), 7.26 (d, J=18.4 Hz, 1H), 3.13 (q, J=7.5 Hz, 2H), 1.45 (s, 12H), 1.26 (t, J=7.5 Hz, 3H).

### Intermediate A-34, tert-butyl N-(4-chloro-3-cyano-7-fluoro-thieno[3,2-c]pyridin-2-yl)carbamate

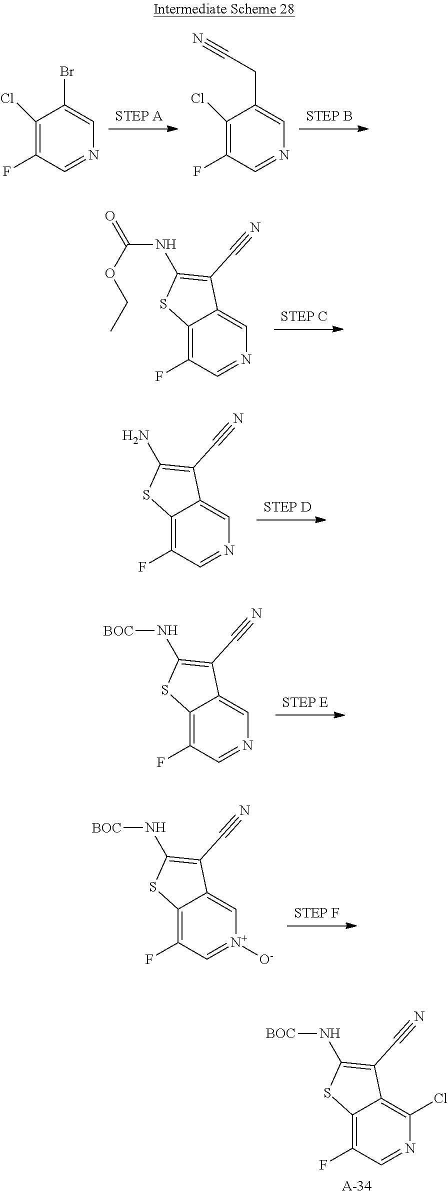

Step A, 2-(4-chloro-5-fluoro-3-pyridyl) acetonitrile. A solution of 3-Bromo-4-chloro-5-fluoropyridine (500. mg, 2.38 mmol), 4-(4,4,5,5-Tetramethyl-1,3,2-dioxaborolan-2-yl)-1,2-oxazole (556.06 mg, 2.85 mmol), and Potassium fluoride (414.15 mg, 7.13 mmol) in Water (0.5 mL) and DMSO (5 mL) was sparged with nitrogen for 15 mins. [1,1′-Bis(di-tert-butylphosphino)ferrocene]dichloropalladium(II) (154.86 mg, 0.24 mmol) was added to the mixture then heated to 130° C. using microwave irradiation for 30 mins, then cooled to rt, filtered through a pad of celite, diluted with EtOAc and purified by silica gel chromatography (12 g, eluting in 50% EtOAc in Pet. Ether) the desired fractions were combined to give 2-(4-chloro-5-fluoro-3-pyridyl) acetonitrile (273 mg, 1.6005 mmol, 67% yield) as a brown glass.

UPLC-MS (ES⁺, Method 5): 1.32 min, m/z 170.9 [M+H]⁺

Step B, ethyl N-(3-cyano-7-fluoro-thieno[3,2-c]pyridin-2-yl)carbamate. To a stirring solution of 2-(4-chloro-5-fluoro-3-pyridyl) acetonitrile (336. mg, 1.97 mmol) in THF (10 mL) under nitrogen at 0° C. Sodium hydride, (60% dispersed in mineral oil) (118.18 mg, 2.95 mmol) was added portionwise and stirred for 15 mins. Ethoxycarbonyl isothiocyanate (0.26 mL, 2.17 mmol) was added and the mixture stirred at 0° C. for 5 mins before being brought to room temperature and stirred for a further 10 mins. Copper (I) iodide (37.52 mg, 0.2 mmol) and L-Proline (45.36 mg, 0.39 mmol) were added and the reaction mixture heated to 65° C. for 1 h. The reaction mixture was cooled to rt and quenched by the addition of water, and EtOAc added. The phases were separated and the aqueous phase was washed with EtOac (×2). The combined organic fractions were dried over Na₂SO₄, filtered and concentrated under reduced pressure. The crude material was purified by silica gel chromatography (12 g, 0-100% EtOac in pet. ether then 0-20% MeOH in EtOAc) the desired fractions were combined and concentrated under reduced pressure to give ethyl N-(3-cyano-7-fluoro-thieno[3,2-c]pyridin-2-yl)carbamate (325 mg, 1.2252 mmol, 62% yield) as a dark yellow powder.

UPLC-MS (ES+, Method 5): 1.65 min, m/z 265.9 [M+H]⁺

Step C, 2-amino-7-fluoro-thieno[3,2-c]pyridine-3-carbonitrile. To a solution of ethyl N-(3-cyano-7-fluoro-thieno[3,2-c]pyridin-2-yl)carbamate (500. mg, 1.88 mmol) in DMSO (4 mL) at 0° C. was added a solution of Sodium hydroxide (753.92 mg, 18.85 mmol) in Water (4 mL) dropwise. The reaction mixture was stirred for 15 min, then heated to 105° C. and stirred for a further 1.5 h. The reaction mixture was then cooled to rt and poured into an ice/water mix (80 mL) and stirred until all the ice had melted. The resulting suspension was filtered through a pad of celite washing with water then methanol/DCM. the organic fraction was concentrated under reduced pressure and dried in a vacuum oven to give 2-amino-7-fluoro-thieno[3,2-c]pyridine-3-carbonitrile (110 mg, 0.5694 mmol, 30% yield) as a yellow powder.

UPLC-MS (ES+, Method 5): 1.16 min, m/z 193.9 [M+H]⁺

Step D, tert-butyl N-(3-cyano-7-fluoro-thieno[3,2-c]pyridin-2-yl)carbamate. To a solution of 2-amino-7-fluoro-thieno[3,2-c]pyridine-3-carbonitrile (175 mg, 0.91 mmol) in DCM (2 mL) and DMF (2 mL) at 0° C. was added N, N-Diisopropylethylamine (0.32 mL, 1.81 mmol), 4-dimethylamino pyridine, DMAP (11.07 mg, 0.09 mmol), and di-tert-butyl dicarbonate (296.53 mg, 1.36 mmol). The reaction mixture was warmed to room temperature and stirred for 2 h. The reaction mixture was concentrated under reduced pressure and the crude material was purified by silica gel chromatography (12 g, 10-70% EtOAc in pet. ether) the desired fractions were combined and concentrated under reduced pressure to give tert-butyl N-(3-cyano-7-fluoro-thieno[3,2-c]pyridin-2-yl)carbamate (115 mg, 0.3921 mmol, 43% yield) as yellow powder.

UPLC-MS (ES+, Method 5): 1.92 min, m/z 293.9 [M+H]⁺

¹H NMR (400 MHz, DMSO-d6) δ/ppm: 12.31 (1H, s), 8.74 (1H, s), 8.49 (1H, s), 1.53 (9H, s)

Step E, tert-butyl N-(3-cyano-7-fluoro-5-oxido-thieno[3,2-c]pyridin-5-ium-2-yl)carbamate. m-chloroperbenzoic acid, MCPBA (703.63 mg, 4.08 mmol) was added to a solution of tert-butyl N-(3-cyano-7-fluoro-thieno[3,2-c]pyridin-2-yl)carbamate (598. mg, 2.04 mmol) in DCM (15 mL) and the reaction mixture was stirred at room temperature for 2 h. The reaction mixture was concentrated under reduced pressure and the crude material was purified by silica gel chromatography (12 g, 0-6% MeOH in DCM) the desired fractions were combined and concentrated under reduced pressure to give tert-butyl N-(3-cyano-7-fluoro-5-oxido-thieno[3,2-c]pyridin-5-ium-2-yl)carbamate (507 mg, 1.6391 mmol, 80% yield) as an off-white powder.

UPLC-MS (ES+, Method 5): 1.56 min, m/z 309.9 [M+H]⁺

¹H NMR (400 MHz, DMSO-d6) δ/ppm: 12.45 (1H, br. s), 8.54 (1H, dd, J=5.4, 1.4 Hz), 8.35 (1H, d, J=1.4 Hz), 1.53 (9H, s)

Step F, tert-butyl N-(4-chloro-3-cyano-7-fluoro-thieno[3,2-c]pyridin-2-yl)carbamate (A-34). Phosphorus oxychloride (0.09 mL, 1. mmol) was added to a solution of tert-butyl N-(3-cyano-7-fluoro-5-oxido-thieno[3,2-c]pyridin-5-ium-2-yl)carbamate (155 mg, 0.5 mmol) and DMF (0.05 mL) in Toluene (5 mL) and the reaction mixture was left to stir at room temperature for 2 h. The reaction was quenched by dropwise addition to sat.aq. NaHCO₃ (10 mL) adding solid NaHCO₃as needed to maintain pH >7. The reaction vessel was rinsed with DCM (2*15 mL) adding to the aq solution. The phases were separated and the aqueous phase washed with DCM (15 mL). The combined organic fractions were dried over Na₂SO₄, filtered, concentrated under reduced pressure. The crude material was purified by silica gel chromatography (12 g, 50-100% DCM in pet. ether) the desired fractions were combined to give tert-butyl N-(4-chloro-3-cyano-7-fluoro-thieno[3,2-c]pyridin-2-yl)carbamate (135 mg, 0.4119 mmol, 82% yield) as a colourless solid

UPLC-MS (ES+, Method 5): 2.10 min, m/z 327.9 [M+H]⁺¹H NMR (400 MHz, DMSO-d6) δ/ppm: 12.23 (1H, br. s), 8.34 (1H, s), 1.54 (9H, s)

### Intermediate I-1, 4-bromo-7,9-dichloro-2-methyl-pyrazolo[4,3-f]quinazoline

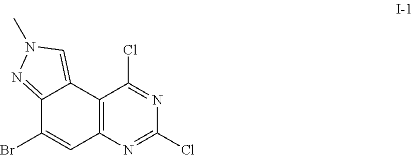 [SMILES: Cn1cc2c(n1)c(Br)cc1nc(Cl)nc(Cl)c12]

Intermediate I-1 was prepared following the route in Intermediate Scheme 29 below.

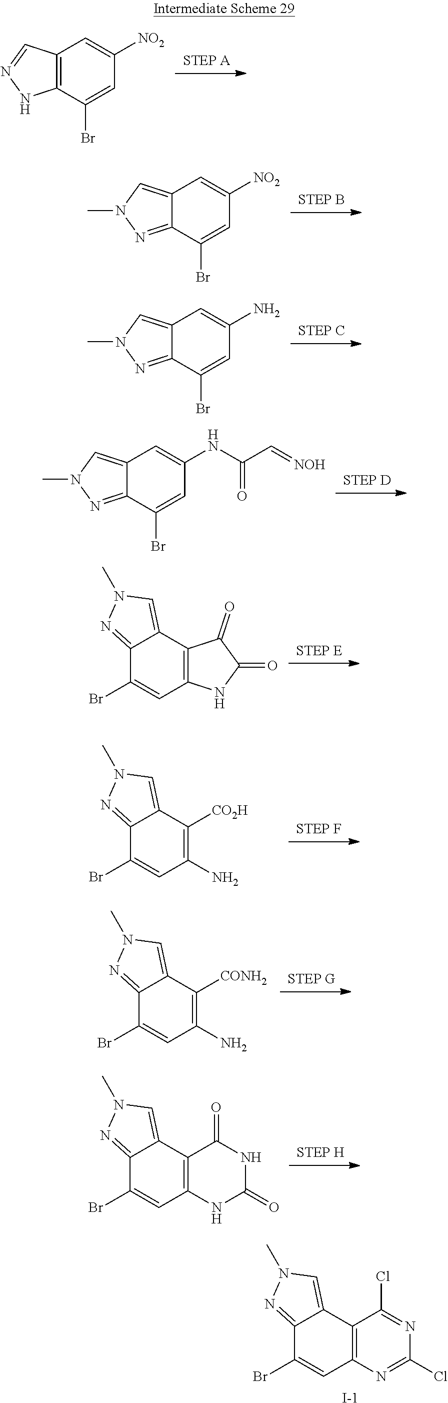

Step A, 7-bromo-2-methyl-5-nitro-indazole. Potassium carbonate (2.3 g, 16.53 mmol) was added to 7-Bromo-5-nitro-1H-indazole (4 g, 16.53 mmol) in DMF (41 mL), followed by iodomethane (3.1 mL, 49.58 mmol). The reaction was stirred at room temperature overnight. The solvent was removed in vacuo and the crude material dissolved in EtOAc and water. The phases were separated and the aqueous was extracted with EtOAc (3×50 mL), dried with Na₂SO₄, filtered and concentrated. The crude was taken up in DCM, loaded onto silica and purified by flash column chromatography (220 g SiO₂, 15-75% EtOAc in petroleum ether). The desired fractions were concentrated to afford 7-bromo-2-methyl-5-nitro-indazole (3.58 g, 13.98 mmol, 38% yield) as a dark orange solid.

UPLC-MS (ES⁺, Method 2): 1.55 min, m/z 256.0, 257.9 [M+H]⁺.

¹H NMR (400 MHz, CDCl₃)/ppm: 8.69 (d, J=2.0 Hz, 1H), 8.37 (d, J=2.0 Hz, 1H), 8.31 (s, 1H), 4.34 (s, 3H).

Step B, 7-bromo-2-methyl-indazol-5-amine. A solution of 7-bromo-2-methyl-5-nitro-indazole (3.58 g, 13.98 mmol) in ethyl acetate (93 mL) was purged 3× with vacuum/N₂, and platinum on carbon, 5 wt. % loading (272 mg, 1.4 mmol) was added. The mixture was then purged 3× with vacuum/H₂ and heated to 60° C. overnight. The reaction was purged with N₂, cooled to room temperature then filtered through a Celite pad, washing with EtOAc. The filtrate was concentrated to afford 7-bromo-2-methyl-indazol-5-amine (3.43 g, 13.98 mmol, 100% yield) as a brown solid.

UPLC-MS (ES⁺, Method 2): 0.56 min, 225.9, 227.9 [M+H]⁺.

¹H NMR (400 MHz, CDCl₃) δ/ppm: 7.71 (s, 1H), 7.07 (d, J=1.9 Hz, 1H), 6.72 (d, J=1.9 Hz, 1H), 4.18 (s, 3H), 3.60 (br s, 2H).

Step C, N-(7-bromo-2-methyl-indazol-5-yl)-2-hydroxyimino-acetamide. To a solution of sodium sulfate (38.8 g, 273 mmol) in water (69.887 mL), was added chloral hydrate (2.76 g, 16.7 mmol) at room temperature. To the resulting solution was added 7-bromo-2-methyl-indazol-5-amine (3.43 g, 15.2 mmol) in hydrogen chloride (1M in water) (16.7 mL, 16.7 mmol). The mixture turned cloudy and hydroxylamine hydrochloride (3.37 g, 48.6 mmol) was added. The mixture was heated to 80° C. for 2 hours, then returned to room temperature, filtered, and the solid collected to give N-(7-bromo-2-methyl-indazol-5-yl)-2-hydroxyimino-acetamide (3.13 g, 10.5 mmol, 69% yield) as a black-brown solid after drying in the vacuum oven.

UPLC-MS (ES⁺, Method 2): 1.16 min, m/z 296.9, 298.9 [M+H]⁺.

¹H NMR (400 MHz, DMSO-d6) δ/ppm: 12.17 (s, 1H), 10.20 (s, 1H), 8.46 (s, 1H), 8.16 (d, J=1.7 Hz, 1H), 7.76 (d, J=1.6 Hz, 1H), 7.65 (s, 1H), 4.18 (s, 3H).

Step D, 4-bromo-2-methyl-6H-pyrrolo[3,2-e]indazole-7,8-dione. N-(7-bromo-2-methyl-indazol-5-yl)-2-hydroxyimino-acetamide (3.13 g, 10.53 mmol) was dissolved in sulfuric acid (19.7 mL, 368.7 mmol) and heated to 60° C. for 2 hours. The reaction was then cooled to room temperature and poured into ice-water. The precipitate was collected by filtration and dried in the vacuum oven, to afford 4-bromo-2-methyl-6H-pyrrolo[3,2-e]indazole-7,8-dione (2.31 g, 8.25 mmol, 78% yield) as a black-purple solid.

UPLC-MS (ES⁺, Method 2): 1.20 min, m/z 279.9 [M+H]⁺.

¹H NMR (400 MHz, DMSO-d₆) δ/ppm: 10.81 (s, 1H), 8.58 (s, 1H), 7.25 (s, 1H), 4.19 (s, 3H).

Step E, 5-amino-7-bromo-2-methyl-indazole-4-carboxylic acid. To a solution of 4-bromo-2-methyl-6H-pyrrolo[3,2-e]indazole-7,8-dione (2.31 g, 8.26 mmol) in 1,4-dioxane (41.3 mL) was added sodium hydroxide (19M in water) (4.35 mL, 82.6 mmol) at 0° C. Hydrogen peroxide solution 30 wt % in H₂O (4.22 mL, 41.29 mmol) was added dropwise. The solution was returned to room temperature and stirred for 2 hours. The reaction was quenched with sat. Na₂S₂O₃ (5 mL) and adjusted to pH 1 with 2N HCl (aq). The volatiles were removed in vacuo. The solid was extracted with n-BuOH (3×10 mL), and the organic phase concentrated to afford 5-amino-7-bromo-2-methyl-indazole-4-carboxylic acid (1.63 g, 6.04 mmol, 73% yield) as a brown solid.

UPLC-MS (ES⁺, Method 2): 1.20 min, m/z 269.9, 271.8 [M+H]⁺.

¹H NMR (400 MHz, MeOD) δ/ppm: 8.42 (s, 1H), 7.16 (s, 1H), 4.14 (s, 3H).

Step F, 5-amino-7-bromo-2-methyl-indazole-4-carboxamide. To a solution of 5-amino-7-bromo-2-methyl-indazole-4-carboxylic acid (1.61 g, 5.96 mmol), ammonium chloride (3.2 g, 59.6 mmol), and N,N-Diisopropylethylamine (3.11 mL, 17.9 mmol) in DMF (39.7 mL) was added HATU (2.26 g, 5.96 mmol), and stirred at room temperature for 5 hours. The solvent was removed in vacuo, and the residue suspended in water, then extracted with n-butanol (3×20 mL). The solvent was removed in vacuo to afford 5-amino-7-bromo-2-methyl-indazole-4-carboxamide (1.07 g, 3.99 mmol, 67% yield) as a brown solid.

UPLC-MS (ES⁺, Method 2): 1.25 min, m/z 268.9, 270.9 [M+H]⁺.

Step G, 4-bromo-2-methyl-6H-pyrazolo[4,3-f]quinazoline-7,9-dione. 1,1′-Carbonyldiimidazole (970.7 mg, 5.99 mmol) was added to a solution of 5-amino-7-bromo-2-methyl-indazole-4-carboxamide (1.07 g, 3.99 mmol) in THF (26.6 mL), and heated to reflux for 3 hours. The solvent was removed in vacuo, and the residue suspended in 1M HCl. The precipitate was collected by filtration to afford 4-bromo-2-methyl-6H-pyrazolo[4,3-f]quinazoline-7,9-dione (418 mg, 1.42 mmol, 36% yield) as a brown solid.

UPLC-MS (ES⁺, Method 2): 1.07 min, m/z 294.9, 296.9 [M+H]⁺.

¹H NMR (400 MHz, DMSO-d6) δ/ppm: 11.44 (s, 1H), 11.32 (s, 1H), 8.76 (s, 1H), 8.19 (s, 1H), 4.22 (s, 3H).

Step H, 4-bromo-7,9-dichloro-2-methyl-pyrazolo[4,3-f]quinazoline (1-1). Phosphorus oxychloride (1.22 mL, 13.1 mmol) was added to a suspension of 4-bromo-2-methyl-6H-pyrazolo[4,3-f]quinazoline-7,9-dione (155 mg, 0.53 mmol) in dimethylaniline (0.33 mL, 2.63 mmol), the reaction tube was sealed, and heated to 75° C. overnight. The reaction was cooled to room temperature, and slowly added to an ice-cold sat. NaHCO₃ solution. The precipitate was collected by filtration and dried under vacuum to afford 4-bromo-7,9-dichloro-2-methyl-pyrazolo[4,3-f]quinazoline (89 mg, 0.27 mmol, 51% yield) as a black-green solid.

UPLC-MS (ES⁺, Method 2): 1.82 min, m/z 332.6 [M+H]⁺.

### Intermediate I-55, 4-bromo-7,9-dichloro-3-methyl-pyrazolo[4,3-f]quinazoline

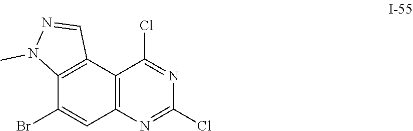 [SMILES: Cn1ncc2c3c(Cl)nc(Cl)nc3cc(Br)c21]

Intermediate I-55 was prepared by analogy with I-1 (intermediate scheme 29) above, using the other regio-isomer formed during step A.

### Intermediate I-49, 4-bromo-7,9-dichloro-2-cyclopropyl-pyrazolo[4,3-f]

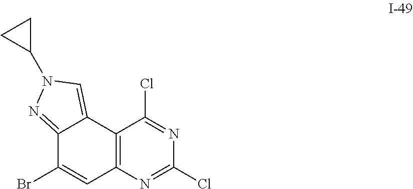 [SMILES: Clc1nc(Cl)c2c(cc(Br)c3nn(C4CC4)cc32)n1]

Intermediate I-49 was prepared by analogy with I-1 (intermediate scheme 29) above, with the following variation on Step A shown below.

Step A, 7-bromo-2-cyclopropyl-5-nitro-indazole. A solution of 7-Bromo-5-nitro-1H-indazole (19.2 g, 79.33 mmol), copper (II) acetate (43.23 g, 237.99 mmol), cyclopropylboronic acid (20.44 g, 237.99 mmol), DMAP (38.77 g, 317.32 mmol) and Pyridine (25.66 mL, 317.32 mmol) in 1,4-Dioxane (800 mL) was left to stir under oxygen at 100° C. overnight. The mixture was diluted with water (1000 ml) and extracted with EtOAc (300 ml×3). The combined organic phases were washed with brine, dried over sodium sulfate and concentrated under vacuum. The crude residue was purified by silica gel chromatography (eluting with 1/3 EA/PE) to afford 7-bromo-2-cyclopropyl-5-nitro-indazole as a yellow solid (4.2 g, 18% yield).

LCMS (ES-API, Method 4) 0.77 min, 282.0/284.0 [M+H]⁺.

### Intermediate I-52, 4-bromo-7,9-dichloro-5-fluoro-2-methyl-pyrazolo[4,3-f]quinoline

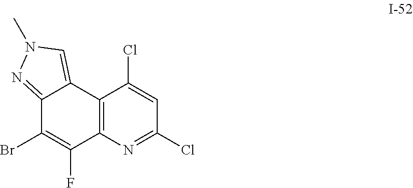 [SMILES: Cn1cc2c(n1)c(Br)c(F)c1nc(Cl)cc(Cl)c12]

Intermediate 1-52 was prepared following the route in Intermediate Scheme 30 below.

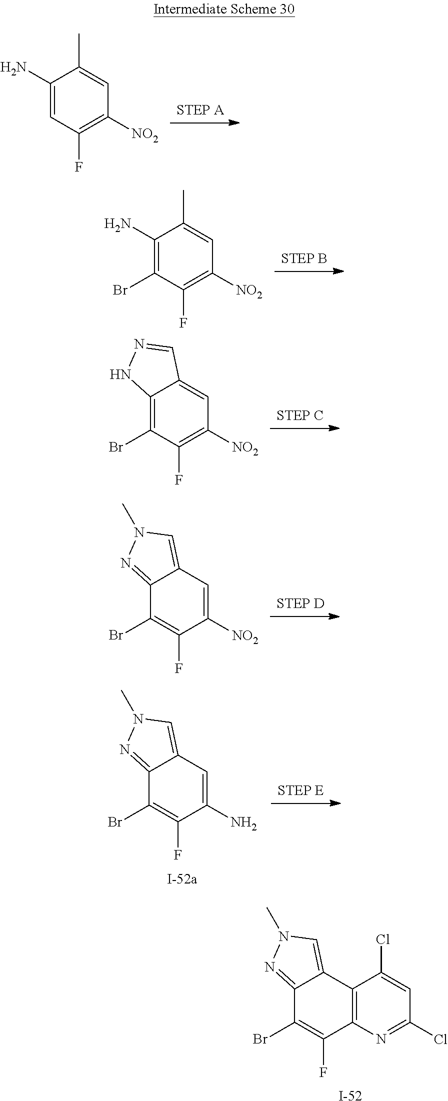

Step A, 2-bromo-3-fluoro-6-methyl-4-nitro-aniline. A stirring mixture of 5-fluoro-2-methyl-4-nitro-aniline (49 g, 288. mmol) and NBS (76.8, 432 mmol) in MeCN (1 L) was heated to 65° C. and left to stir for 2 hours. The mixture was concentrated under vacuum and the residue poured into DCM (2 L) and washed with NaOH (1N, 1 L). The organic layers were combined and concentrated to afford 2-bromo-3-fluoro-6-methyl-4-nitro-aniline to give (60 g, 240.93 mmol, 84% yield) as a yellow solid.

LC-MS (ES-API, Method 4): 1.84 min, 249.0 [M+H]⁺.

Step B, 7-bromo-6-fluoro-5-nitro-1H-indazole. To a stirring mixture of 2-bromo-3-fluoro-6-methyl-4-nitro-aniline (60. g, 240.93 mmol) in Chloroform (600 mL) was added Potassium acetate (28.37 g, 289.11 mmol). The mixture was cooled to 0° C. and Acetic Anhydride (68.32 mL, 722.78 mmol) added dropwise. The mixture was left to stir at 0° C. for 1 hour after which time isopentyl nitrite (64.73 mL, 481.85 mmol) was added. The reaction mixture was then heated to 65° C. and left to stir overnight. The reaction mixture was cooled to room temperature and concentrated under vacuum before being treated with Na₂CO₃ (aq) and extracted with DCM (600 mL×3). The organic layers were concentrated under vacuum, poured into THF (600 mL) and NaOH (aq, 2M) added. The resulting mixture was stirred at 0° C. for 2 hours. The mixture was concentrated under vacuum and the residue extracted with DCM (600 mL×3). The organic layers were concentrated under vacuum and purified by silica gel chromatography (eluting with 3% MeOH in DCM) to afford 7-bromo-6-fluoro-5-nitro-1H-indazole (17 g, 32.69 mmol, 14% yield) as a red solid.

LC-MS (ES-API, Method 4): 1.52 min, 259.9 [M+H]⁺.

Step C, 7-bromo-6-fluoro-2-methyl-5-nitro-indazole. To a stirring solution of 7-bromo-6-fluoro-5-nitro-1H-indazole (5. g, 9.61 mmol) in Ethyl acetate (50 mL) was added Trimethyloxonium tetrafluoroborate (1.85 g, 12.5 mmol) and the reaction left to stir at room temperature overnight. The reaction mixture was treated with water (60 mL) and extracted with EtOAc (60 mL×3). The organic layers were concentrated under vacuum to afford 7-bromo-6-fluoro-2-methyl-5-nitro-indazole (5 g, 9.1224 mmol, 95% yield) as an orange solid.

LC-MS (ES-API, Method 4): 1.64 min, 274.0 [M+H]⁺.

Step D, 7-bromo-6-fluoro-2-methyl-indazol-5-amine. To a stirring mixture of 7-bromo-6-fluoro-2-methyl-5-nitro-indazole (5.0 g, 9.12 mmol) in Ethanol (50 mL) and Water (50 mL) was added Fe (2.55 g, 45.61 mmol) and NH₄Cl (2.44 g, 45.61 mmol). The mixture was heated to 80° C. and left to stir for 2 hours. The mixture was filtered and extracted with EtOAc (500 mL×3). The organic layers were concentrated under vacuum and purified by silica gel chromatography (eluting with 35% EtOAc in Pet. Ether) to afford 7-bromo-6-fluoro-2-methyl-indazol-5-amine (1.5 g, 6.1458 mmol, 67% yield) as a red solid.

LC-MS (ES-API, Method 4): 0.81 min, 244.0 [M+H]⁺.

Step E, 4-bromo-7,9-dichloro-5-fluoro-2-methyl-pyrazolo[4,3-f]quinoline (I-52). A mixture of 7-bromo-6-fluoro-2-methyl-indazol-5-amine (1.5 g, 6.15 mmol) and malonic ccid (1.28 g, 12.29 mmol) in POCl₃ (20 mL) was heated to 110° C. and left to stir for 3 hours. The reaction mixture was concentrated under vacuum, diluted with water (200 mL) and extracted with EtOAc (200 mL×3). The combine organic phases were washed with brine, dried over sodium sulfate, concentrated under vacuum and purified by silica gel column (Pet.ether:EtOAc 1:1) to afford 4-bromo-7,9-dichloro-5-fluoro-2-methyl-pyrazolo[4,3-f]quinoline (560 mg, 1.6047 mmol, 26% yield) as a yellow solid.

LC-MS (ES-API, Method 4) 2.36 min, 347.9 [M+H]⁺

### Intermediate 1-72, 4-bromo-7,9-dichloro-2-(2,2,2-trifluoroethyl)pyrazolo[4,3-f]quinazoline

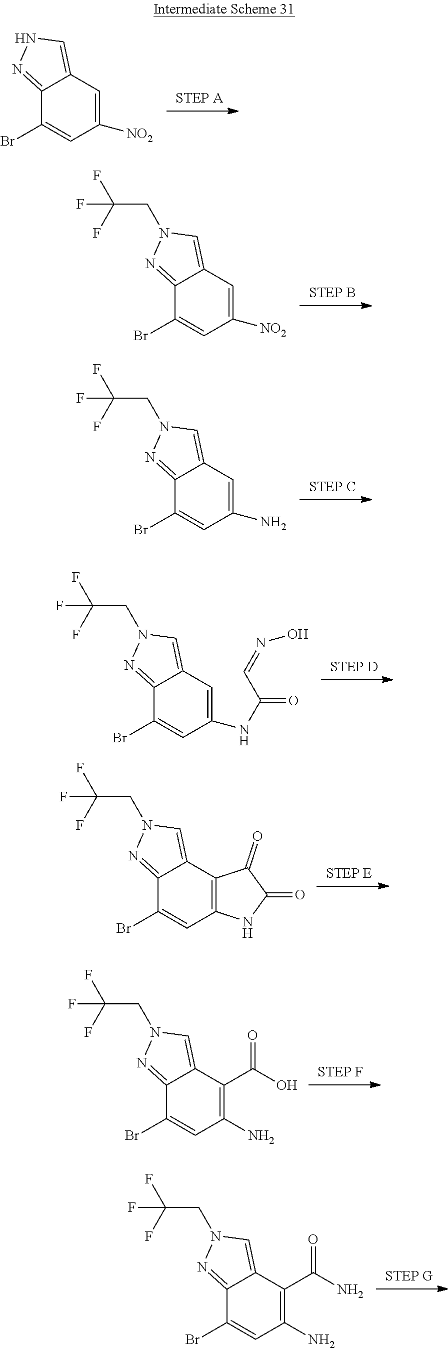

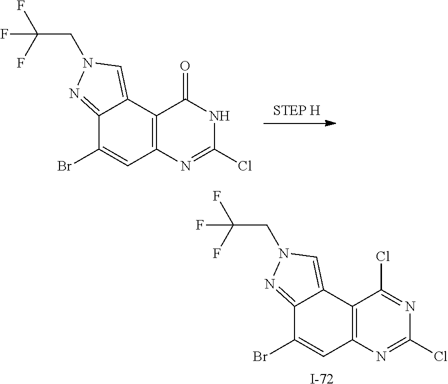

Step A, 7-bromo-5-nitro-2-(2,2,2-trifluoroethyl) indazole. To a stirring solution of 7-Bromo-5-nitro-1H-indazole (3000 mg, 12.4 mmol) and Cesium Carbonate (8077.18 mg, 24.79 mmol) in DMF (24.79 mL) was added 2,2,2-Trifluoroethyl triflate (3.04 mL, 21.07 mmol). It was stirred at 70° C. for 30 min. Reaction mixture allowed to cool back down to room temperature and water (200 ml) added. This was extracted with EtOAc (3×50 ml), organic extracts combined, washed with water (2×50 mL), saturated solution of brine (50 mL), dried over sodium sulfate, filtered, and evaporated. It was purified by silica chromatography eluting 0-40% EtOAc in petroleum ether to give the product 7-bromo-5-nitro-2-(2,2,2-trifluoroethyl) indazole (2594.6 mg, 8.01 mmol, 65% yield) as a brown solid.

UPLC-MS (ES⁺, Method 2): 1.77 min, m/z 326.0 [M+H]⁺.

¹H NMR (400 MHz, CDCl₃) δ/ppm: 8.73 (d, J=1.8 Hz, 1H), 8.51 (s, 1H), 8.40 (dd, J=1.9, 0.7 Hz, 1H), 5.15 (q, J=8.1 Hz, 2H).

Step B, 7-bromo-2-(2,2,2-trifluoroethyl) indazol-5-amine. To a suspension of 7-bromo-5-nitro-2-(2,2,2-trifluoroethyl) indazole (2594.6 mg, 8.01 mmol) in Ethanol (20.016 mL)/Water (6.6721 mL) were successively added ammonium chloride (1.28 g, 24.02 mmol) and Iron (1.34 g, 24.02 mmol). The mixture was heated to 80° C. and vigorously stirred for 45 min. Additional Iron (1341.5 mg, 24.02 mmol) was added. Reaction mixture was allowed to stir at 80° C. for 1 hr. The mixture was cooled to room temperature and passed through a plug of celite. The filter cake was washed with ethyl acetate (2×30 mL). Filtrate collected and transferred to a separatory funnel. Water (30 mL) was added and layers partitioned. Organic layer separated and aqueous layer extracted with EtOAc (20 mL). Organic layers combined, washed with a saturated solution of brine (20 mL), dried over sodium sulfate, filtered and concentrated under reduced pressure. Purification by flash column chromatography on silica gel eluting with 20-80% EtOAc in petroleum ether to afford 7-bromo-2-(2,2,2-trifluoroethyl) indazol-5-amine (1838.2 mg, 6.2507 mmol, 78% yield) as an off-white solid.

UPLC-MS (ES⁺, Method 5): 1.30 min, m/z 295.9 [M+H]⁺.

¹H NMR (400 MHz, CDCl₃) δ/ppm: 7.86 (s, 1H), 7.13 (d, J=1.9 Hz, 1H), 6.70 (d, J=1.9 Hz, 1H), 4.99 (q, J=8.3 Hz, 2H), 3.65 (br s, 2H).

Step C, (2E)-N-[7-bromo-2-(2,2,2-trifluoroethyl) indazol-5-yl]-2-hydroxyimino-acetamide. To a suspension of Chloral hydrate (2.06 g, 12.5 mmol), 7-bromo-2-(2,2,2-trifluoroethyl) indazol-5-amine (1838.2 mg, 6.25 mmol) and Sodium sulfate (7.1 g, 50.01 mmol) in a mixture of DMF (9.92 mL) and Water (21.3 mL) was added Hydrogen Chloride (2.82 mL, 34.4 mmol) (conc HCl). Reaction mixture allowed to stir at 90° C. for 1 hr. Hydroxylamine hydrochloride (1.52 g, 21.88 mmol) was added. Reaction mixture allowed to stir at 90° C. for 20 min. Reaction mixture allowed to cool back down to room temperature. Partitioned between a layer of EtOAc (80 mL) and water (80 mL). Organic layer separated and aqueous layer extracted with EtOAc (2×40 mL). Organic layers combined, washed with water (2×50 mL), a saturated solution of brine (50 mL), dried over sodium sulfate, filtered and concentrated under reduced pressure to afford (2E)-N-[7-bromo-2-(2,2,2-trifluoroethyl) indazol-5-yl]-2-hydroxyimino-acetamide (2.28 g, 6.25 mmol, 100% yield) as a brown solid.

UPLC-MS (ES+, Method 5): 1.56 min, m/z 366.9 [M+H]⁺.

Step D, 4-bromo-2-(2,2,2-trifluoroethyl)-6H-pyrrolo[3,2-e]indazole-7,8-dione. A mixture of (2E)-N-[7-bromo-2-(2,2,2-trifluoroethyl) indazol-5-yl]-2-hydroxyimino-acetamide (2.28 g, 6.25 mmol) and sulfuric acid (11.66 mL, 218.78 mmol) was allowed to stir at 70° C. for 3 hrs. Reaction mixture allowed to cool back down to room temperature. Ice-water (100 mL) was added. Solid precipitate collected by vacuum filtration over filter paper. Paper washed with water (2×20 mL). Solid precipitate dried under vacuum to afford 4-bromo-2-(2,2,2-trifluoroethyl)-6H-pyrrolo[3,2-e]indazole-7,8-dione (962.4 mg, 2.7649 mmol, 65% yield) as a brown solid.

UPLC-MS (ES+, Method 5): 1.59 min, m/z 347.9 [M+H]⁺

¹H NMR (400 MHz, CDCl₃) δ/ppm: 8.40 (s, 1H), 7.33 (s, 1H), 5.06 (q, J=8.1 Hz, 2H).

Step E, 5-amino-7-bromo-2-(2,2,2-trifluoroethyl) indazole-4-carboxylic acid. To an ice-cooled suspension of 4-bromo-2-(2,2,2-trifluoroethyl)-6H-pyrrolo[3,2-e]indazole-7,8-dione (1827.6 mg, 5.25 mmol) in 1,4-Dioxane (23.8 mL) was added a solution of sodium hydroxide (2100 mg, 52.51 mmol) in Water (2.50 mL). Then Hydrogen peroxide solution (30 wt % in H₂O) (2.68 mL, 26.25 mmol) was added. Reaction mixture allowed to stir at room temperature for 30 min. Reaction mixture quenched with a saturated solution of sodium thiosulphate. Then acidified to ~pH2 with 2M HCl (aq). Partitioned between a layer of EtOAc (50 mL) and water (50 mL). Organic layer separated, aqueous layer extracted with EtOAc (2×20 mL) and organic layers combined. Washed with a saturated solution of brine (20 mL), dried over sodium sulfate, filtered and concentrated under reduced pressure to afford 5-amino-7-bromo-2-(2,2,2-trifluoroethyl) indazole-4-carboxylic acid (1775.1 mg, 5.25 mmol, 100% yield) as a brown solid.

UPLC-MS (ES+, Method 5): 1.56 min, m/z 339.9 [M+H]⁺.

¹H NMR (400 MHz, CDCl₃) δ/ppm: 8.24 (s, 1H), 7.06 (s, 1H), 6.27 (br s, 1H), 5.05 (br s, 1H), 4.97 (q, J=9.1 Hz, 2H).

Step F, 5-amino-7-bromo-2-(2,2,2-trifluoroethyl) indazole-4-carboxamide. HATU (2395.7 mg, 6.3 mmol) was added to a mixture of 5-amino-7-bromo-2-(2,2,2-trifluoroethyl) indazole-4-carboxylic acid (1775.1 mg, 5.25 mmol), ammonium chloride (2808.6 mg, 52.5 mmol) and N, N-Diisopropylethylamine (2.74 mL, 15.75 mmol) in DMF (15.77 mL). Afterwards, allowed to stir at room temperature for 1 hr. Additional N,N-Diisopropylethylamine (2.74 mL, 15.75 mmol) was added. Reaction mixture allowed to stir at room temperature for 30 min. Extra N,N-Diisopropylethylamine (1.37 mL, 7.88 mmol) was added. Reaction mixture allowed to stir at room temperature for 15 min. Water (150 mL) was added. Solid precipitate collected by vacuum filtration. Solid washed with water (2×20 mL). Solid dried under vacuum to afford 5-amino-7-bromo-2-(2,2,2-trifluoroethyl) indazole-4-carboxamide (969.7 mg, 2.88 mmol, 55% yield) as a black solid.

UPLC-MS (ES+, Method 5): 1.43 min, m/z 338.9 [M+H]⁺.

¹H NMR (400 MHz, CDCl₃) δ/ppm: 8.03 (s, 1H), 7.08 (s, 1H), 5.96 (br s, 1H), 5.64 (br s, 1H), 4.98 (q, J=8.3 Hz, 2H).

Step G, 4-bromo-7-chloro-2-(2,2,2-trifluoroethyl)-8H-pyrazolo[4,3-f]quinazolin-9-one. To a suspension of 5-amino-7-bromo-2-(2,2,2-trifluoroethyl) indazole-4-carboxamide (663.3 mg, 1.97 mmol) in 1,4-Dioxane (7.87 mL) was added thiophosgene (0.38 mL, 4.92 mmol). Reaction mixture allowed to stir at 105° C. for 30 min. Reaction mixture allowed to cool back down to room temperature and concentrated under reduced pressure. Residue triturated with petroleum ether (10 mL) and dried under reduced pressure to afford 4-bromo-7-chloro-2-(2,2,2-trifluoroethyl)-8H-pyrazolo[4,3-f]quinazolin-9-one (750.7 mg, 1.97 mmol, 100% yield) as a brown solid.

UPLC-MS (ES+, Method 2): 1.57 min, m/z 383.0 [M+H]⁺.

Step H, 4-bromo-7,9-dichloro-2-(2,2,2-trifluoroethyl)pyrazolo[4,3-f]quinazoline (1-72). Phosphorus oxychloride (3.18 mL, 34.1 mmol) was added to a suspension of 4-bromo-7-chloro-2-(2,2,2-trifluoroethyl)-8H-pyrazolo[4,3-f]quinazolin-9-one (650.4 mg, 1.7 mmol) and N, N-Diisopropylethylamine (1.19 mL, 6.82 mmol). Afterwards, allowed to stir at 100° C. for 1 hr. Reaction mixture allowed to cool back down to room temperature. Quenched carefully into water. Partitioned between a layer of EtOAc (50 mL) and water (50 mL). Organic layer separated and aqueous layer extracted with EtOAc (20 mL). Organic layers combined, washed with a saturated solution of brine (10 mL), dried over sodium sulfate, filtered and concentrated under reduced pressure. Purification by flash column chromatography on silica gel eluting with 0-60% EtOAc in petroleum ether afforded 4-bromo-7,9-dichloro-2-(2,2,2-trifluoroethyl)pyrazolo[4,3-f]quinazoline (211 mg, 0.528 mmol, 31% yield) (1-72) as a brown solid.

¹H NMR (400 MHz, CDCl₃) δ/ppm: 8.94 (s, 1H), 8.08 (s, 1H), 5.17 (q, J=8.1 Hz, 2H).

UPLC-MS (ES+, Method 2): 1.98 min, m/z 401.0 [M+H]+.

### Intermediate I-75, 4-bromo-7,9-dichloro-2,5-dimethyl-pyrazolo[4,3-f]quinazoline

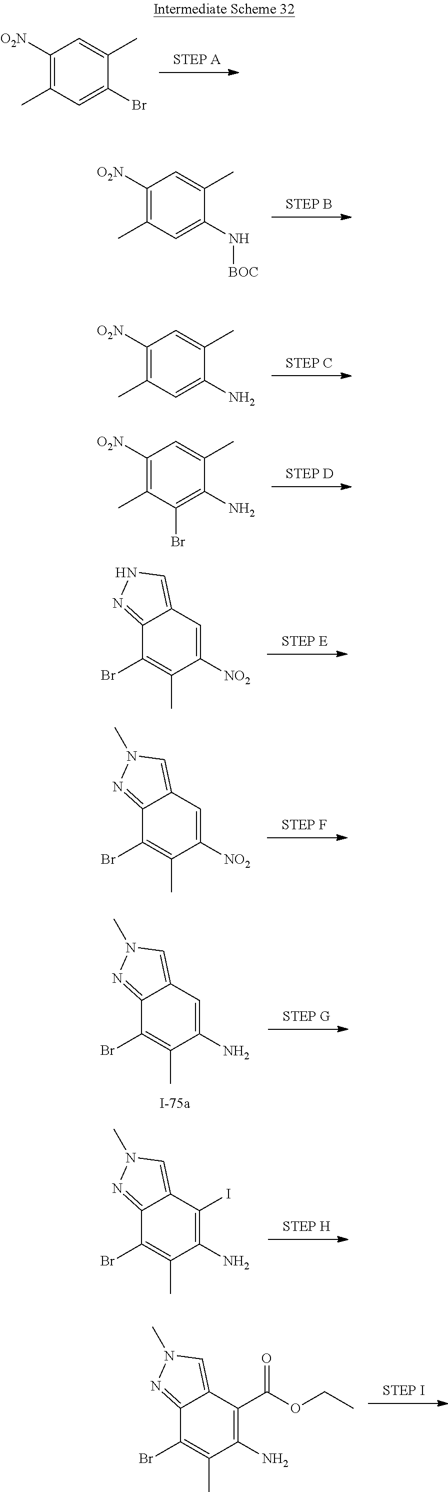

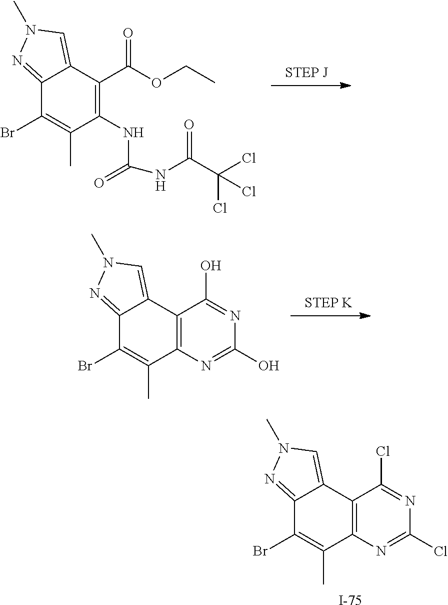

Step A, tert-butyl N-(2,5-dimethyl-4-nitro-phenyl)carbamate. A mixture of 1-bromo-2,5-dimethyl-4-nitro-benzene (50. g, 217.33 mmol) tert-butylcarbamate (28.01 g, 239.07 mmol) X-PHOS (10.36 g, 21.73 mmol) Cs₂CO₃ (106.22 g, 326. mmol) and Pd(OAc)₂ (2.44 g, 10.87 mmol) in 1,4-dioxane (500 mL) was stirred at 100° C. under N₂ for 2 hrs. The reaction mixture was diluted with water (30 ml) and extracted with EtOAc (20 ml×3). The combined organic phases were washed with brine (20 ml×2), dried over sodium sulfate, concentrated under vacuum and purified by silica gel chromatography eluting with 6% EtOAc in petroleum ether to afford tert-butyl N-(2,5-dimethyl-4-nitro-phenyl)carbamate (57.9 g, 179.9 mmol, 90% yield) as a brown solid.

¹H NMR (400 MHz, DMSO-d6) δ/ppm: 8.82 (s, 1H), 7.86 (s, 1H), 7.62 (s, 1H), 2.46 (s, 3H), 2.24 (s, 3H), 1.46 (s, 9H).

Step B, 2,5-dimethyl-4-nitro-aniline. A solution of tert-butyl N-(2,5-dimethyl-4-nitro-phenyl)carbamate (55. g, 206.53 mmol) in DCM (1000 mL) was stirred at room temperature. To the mixture was added Trifluoroacetic acid (200. mL, 2611.75 mmol) and the reaction left to stir at room temperature for 30 min. The pH of the resulting mixture was adjusted to 8 with saturated NaHCO₃ solution, extracted with EtOAc (1000 mL×3), dried over anhydrous Na₂SO₄ and concentrated under vacuum to afford 2,5-dimethyl-4-nitro-aniline (35 g, 210.61 mmol, 100% yield) as a green solid.

¹H NMR (400 MHz, DMSO-d6) δ/ppm: 7.84 (s, 1H), 6.49 (s, 1H), 6.29 (br s, 2H), 2.52 (s, 3H), 2.09 (s, 3H).

Step C, 2-bromo-3,6-dimethyl-4-nitro-aniline. A solution of 2,5-dimethyl-4-nitro-aniline (35. g, 210.61 mmol) and NBS (41.23 g, 231.68 mmol) in MeCN (400 mL) was stirred at 65° C. under N₂ for 2 hrs. The mixture was diluted with water (400 ml) and extracted with EtOAc (100 ml×3). The combined organic phases were washed with brine, dried over sodium sulfate, concentrated under vacuum to afford 2-bromo-3,6-dimethyl-4-nitro-aniline (51 g, 208.1 mmol, 99% yield) as a black solid.

¹H NMR (400 MHz, DMSO-d6) δ/ppm: 7.73 (s, 1H), 6.21 (br s, 2H), 2.55 (s, 3H), 2.18 (s, 3H).

Step D, 7-bromo-6-methyl-5-nitro-1H-indazole. To a stirred solution of 2-bromo-3,6-dimethyl-4-nitro-aniline (50. g, 204.02 mmol) in acetic acid (500 mL) at 0° C. under N₂ was added NaNO₂ (28.15 g, 408.03 mmol) in Water (100 mL) and the mixture was stirred at room temperature overnight. Water (5000 mL) was then added to precipitate a brown solid and filtered. The mixture was concentrated and purified by column eluting 10% MeOH in DCM, desired fractions were combined and evaporated to afford 7-bromo-6-methyl-5-nitro-1H-indazole (25.5 g, 99.59 mmol, 49% yield) as a brown solid.

¹H NMR (400 MHz, DMSO-d6) δ/ppm: 13.92 (s, 1H), 8.56 (s, 1H), 8.45 (s, 1H), 2.63 (s, 3H).

Step E, 7-bromo-2,6-dimethyl-5-nitro-indazole. A solution of 7-bromo-6-methyl-5-nitro-1H-indazole (50. g, 195.27 mmol) in EtOAc (1000 mL) was stirred at room temperature, to the mixture was added Trimethyloxonium tetrafluoroborate (37.55 g, 253.85 mmol) and then the mixture was stirred at room temperature for 3 hrs. The mixture was diluted with NaHCO₃ in water (300 mL) and extracted with EtOAc (100 mL×3). The combined organic phases were washed with brine, dried over sodium sulfate, concentrated under vacuum and purified by silica gel chromatography eluting with 50% EtOAc in petroleum ether, desired fractions were combined and evaporated to afford 7-bromo-2,6-dimethyl-5-nitro-indazole (35 g, 129.59 mmol, 66% yield).

¹H NMR (400 MHz, CDCl₃) δ/ppm: 8.27 (s, 1H), 8.23 (s, 1H), 4.33 (s, 3H), 2.71 (s, 3H).

Step F, 7-bromo-2,6-dimethyl-indazol-5-amine. A solution of 7-bromo-2,6-dimethyl-5-nitro-indazole (30 g, 111.1 mmol), ammonium chloride (29.71 g, 555.4 mmol) and Iron (31.02 g, 555.37 mmol) in Ethanol (300 mL) and Water (150 mL) was stirred at 80° C. under N₂ for 2 hrs. The mixture was diluted with water (3000 mL) and extracted with EtOAc (1000 mL×3). The combined organic phases were washed with brine, dried over sodium sulfate, concentrated under vacuum and purified by silica gel chromatography eluting with 50% EtOAc in petroleum ether, desired fractions were combined and evaporated to afford 7-bromo-2,6-dimethyl-indazol-5-amine (17 g, 70.8 mmol, 64% yield).

¹H NMR (400 MHz, DMSO-d6) δ/ppm: 8.04 (s, 1H), 6.73 (s, 1H), 4.80 (br s, 2H), 4.09 (s, 3H), 2.34 (s, 3H).

Step G, 7-bromo-4-iodo-2,6-dimethyl-indazol-5-amine. N-Iodosuccinimide (1.03 g, 4.58 mmol) was added to a solution of 7-bromo-2,6-dimethyl-indazol-5-amine (1.00 g, 4.16 mmol) in MeCN (20.83 mL) at 0° C. Reaction mixture allowed to stir at that temperature for 15 min. Quenched with a saturated solution of sodium thiosulfate. Partitioned between a layer of EtOAc (20 mL) and water (20 mL). Organic layer separated. Aqueous layer extracted with EtOAc (20 mL). Organic layers combined, washed with water (2×10 mL), saturated solution of brine (10 mL), dried over sodium sulfate, filtered and concentrated under reduced pressure. Purification by flash column chromatography on silica gel eluting with 0-60% EtOAc in petrol afforded 7-bromo-4-iodo-2,6-dimethyl-indazol-5-amine (192.5 mg, 0.526 mmol, 13% yield) as a brown solid.

UPLC-MS (ES+, Method 2): 1.52 min, m/z 368.0 [M+H]⁺

Step H, ethyl 5-amino-7-bromo-2,6-dimethyl-indazole-4-carboxylate. 1,1′-Bis(diphenylphosphino)ferrocene]Palladium(II) chloride dichloromethane complex (42.95 mg, 0.05 mmol) was added to a nitrogen degassed suspension of 7-bromo-4-iodo-2,6-dimethyl-indazol-5-amine (192.5 mg, 0.53 mmol), Carbon monooxide-molybdenum (6:1) (138.85 mg, 0.53 mmol) and 1,8-diazabicyclo[5.4.0]undec-7-ene (0.24 mL, 1.58 mmol) in ethanol (5.20 mL). Afterwards, allowed to stir at 70° C. for 1 hr. Allowed to cool back down to room temperature, filtered over a plug of celite, plug washed with EtOAc (2×10 mL), filtrate collected and all volatiles were removed under reduced pressure. Purification by flash column chromatography on silica gel eluting with 0-60% EtOAc in petroleum ether afforded ethyl 5-amino-7-bromo-2,6-dimethyl-indazole-4-carboxylate (57.8 mg, 0.1852 mmol, 35% yield) as a yellow solid.

¹H NMR (400 MHz, CDCl₃) δ/ppm: 8.01 (s, 1H), 6.47 (br s, 2H), 4.42 (q, J=7.1 Hz, 2H), 4.18 (s, 3H), 2.46 (s, 3H), 1.48 (t, J=7.1 Hz, 3H)

UPLC-MS (ES+, Method 2): 1.58 min, m/z 313.9 [M+H]⁺.

Step I, ethyl 7-bromo-2,6-dimethyl-5-[(2,2,2-trichloroacetyl)carbamoylamino]indazole-4-carboxylate. At room temperature, Trichloroacetyl isocyanate (0.03 mL, 0.23 mmol) was added to a solution of ethyl 5-amino-7-bromo-2,6-dimethyl-indazole-4-carboxylate (57.8 mg, 0.19 mmol) in THF (0.741 mL). After allowing to stir at room temperature for 10 min, all volatiles were removed under reduced pressure and the residue with triturated with diethyl ether (10 mL) for 5 min. Dried under reduced pressure to afford ethyl 7-bromo-2,6-dimethyl-5-[(2,2,2-trichloroacetyl)carbamoylamino]indazole-4-carboxylate (92.7 mg, 0.185 mmol, 100% yield) as an off-white solid.

UPLC-MS (ES+, Method 2): 1.78 min, m/z 501.0 [M+H]⁺.

Step J, 4-bromo-2,5-dimethyl-pyrazolo[4,3-f]quinazoline-7,9-diol. 7M Ammonia in MeOH (0.13 mL, 0.93 mmol) was added to a solution of ethyl 7-bromo-2,6-dimethyl-5-[(2,2,2-trichloroacetyl)carbamoylamino]indazole-4-carboxylate (92.68 mg, 0.19 mmol) in methanol (0.7406 mL) at room temperature. Afterwards, allowed to stir at room temperature for 30 min. More 7M in Ammonia in MeOH (0.13 mL, 0.93 mmol) and Methanol (0.741 mL) were added. Reaction mixture allowed to stir at 50° C. overnight. Reaction mixture allowed to cool back down to room temperature. All volatiles were removed under reduced pressure. The residue was triturated with diethyl ether (10 mL) and dried under reduced pressure to afford 4-bromo-2,5-dimethyl-pyrazolo[4,3-f]quinazoline-7,9-diol (57.24 mg, 0.1852 mmol, 100% yield) as a brown solid.

UPLC-MS (ES+, Method 2): 1.13 min, m/z 310.9 [M+H]⁺.

Step K, 4-bromo-7,9-dichloro-2,5-dimethyl-pyrazolo[4,3-f]quinazoline (I-75). Phosphorus oxychloride (0.43 mL, 4.63 mmol) was added to a mixture of N, N-Diisopropylethylamine (0.06 mL, 0.37 mmol) and 4-bromo-2,5-dimethyl-pyrazolo[4,3-f]quinazoline-7,9-diol (57.24 mg, 0.19 mmol). Afterwards, allowed to stir at 100° C. for 2 hrs. Additional phosphorus oxychloride (0.43 mL, 4.63 mmol) was added and reaction mixture allowed to stir at 100° C. for 3 hrs, room temperature for 10 days and at 100° C. for 1 hr. Reaction mixture allowed to cool back down to room temperature. Quenched carefully into water. Partitioned between a layer of EtOAc (20 mL) and water (20 mL). Organic layer separated and aqueous layer extracted with EtOAc (20 mL). Organic layers combined, washed with a saturated solution of brine (10 mL), dried over sodium sulfate, filtered and concentrated under reduced pressure to afford 4-bromo-7,9-dichloro-2,5-dimethyl-pyrazolo[4,3-f]quinazoline (64.07 mg, 0.185 mmol, 100% yield) (1-75) as a brown oil.

UPLC-MS (ES+, Method 5): 2.12 min, m/z 346.9 [M+H]⁺.

¹H NMR (400 MHz, CDCl₃) δ/ppm: 8.96 (s, 1H), 4.47 (s, 3H), 2.93 (s, 3H).

### Intermediate 1-96, 4-bromo-9-chloro-7-ethylsulfonyl-2,5-dimethyl-pyrazolo[4,3-f]quinazoline

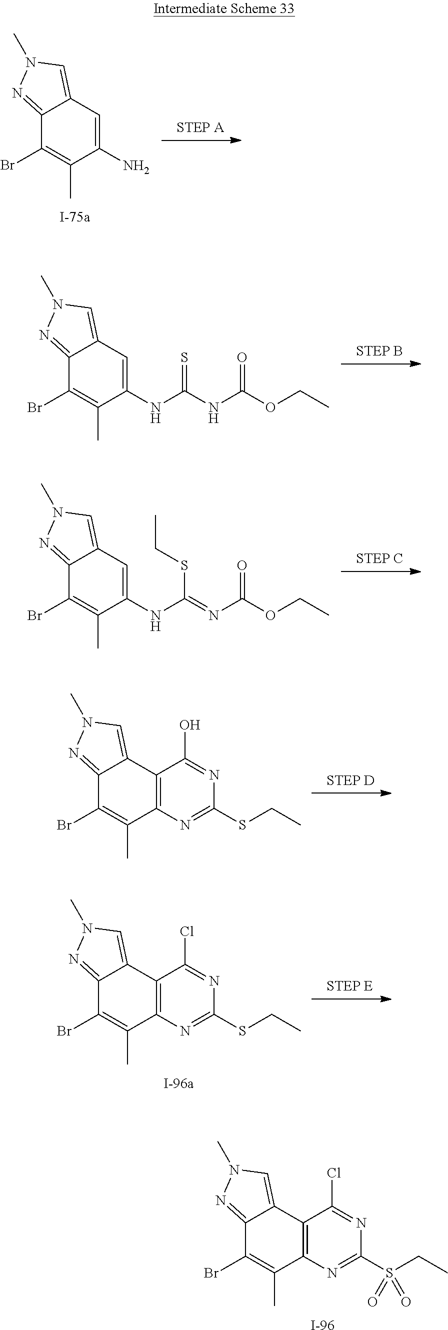

Step A, ethyl N-[(7-bromo-2,6-dimethyl-indazol-5-yl)carbamothioyl]carbamate. To a suspension of 7-bromo-2,6-dimethyl-indazol-5-amine (100 mg, 0.42 mmol) in DCM (2.60 mL) was added ethoxycarbonyl isothiocyanate (0.05 mL, 0.39 mmol) and stirred at 25° C. for 12 hrs. Solvent was evaporated and the crude was purified by flash column chromatography (eluting in 0-100% EtOAc in pet. ether) to give ethyl N-[(7-bromo-2,6-dimethyl-indazol-5-yl)carbamothioyl]carbamate (140 mg, 0.377 mmol, 91% yield) as a yellow powder.

UPLC-MS (ES⁺, Method 5): 1.66 min, m/z 372.9 [M+H]⁺.

¹H NMR (400 MHz, DMSO-d6) δ/ppm: 11.30 (s, 1H), 11.23 (s, 1H), 8.48 (s, 1H), 7.63 (s, 1H), 4.24 (t, J=7.2 Hz, 2H), 4.19 (s, 3H), 2.34 (s, 3H), 1.28 (t, J=7.1 Hz, 3H).

Step B, (NZ)—N-[[(7-bromo-2,6-dimethyl-indazol-5-yl)-ethyl-amino]-ethylsulfanyl-methylene]carbamate. To a solution of ethyl N-[(7-bromo-2,6-dimethyl-indazol-5-yl)carbamothioyl]carbamate (140 mg, 0.38 mmol) in DMF (1.89 mL) under nitrogen, was added Potassium carbonate (156.35 mg, 1.13 mmol) portionwise, followed by dropwise addition of Iodoethane (0.03 mL, 0.38 mmol). Reaction mixture was stirred for 1 hr at room temperature. Mixture was evaporated to give ethyl (NZ)—N-[[(7-bromo-2,6-dimethyl-indazol-5-yl)-ethyl-amino]-ethylsulfanyl-methylene]carbamate (150 mg, 0.351 mmol, 93% yield) as a brown powder. Material was used without further purification.

UPLC-MS (ES⁺, Method 5): 2.03 min, m/z 429.1 [M+H]⁺.

Step C, 4-bromo-7-ethylsulfanyl-2,5-dimethyl-pyrazolo[4,3-f]quinazolin-9-ol. A solution of ethyl (NZ)—N-[[(7-bromo-2,6-dimethyl-indazol-5-yl)amino]-ethylsulfanyl-methylene]carbamate (1320 mg, 3.31 mmol) in NMP (16.6 mL) under nitrogen was stirred for 30 minutes at 175° C. in a microwave. The mixture was diluted with aqueous saturated NaHCO₃ and extracted with EtOAc, washed with water, dried over magnesium sulfate, filtered and evaporated to give 4-bromo-7-ethylsulfanyl-2,5-dimethyl-pyrazolo[4,3-f]quinazolin-9-ol (1120 mg, 3.171 mmol, 96% yield) as a brown oil. Material was used without further purification.

UPLC-MS (ES⁺, Method 5): 1.84 min, m/z 352.9 [M+H]⁺.

Step D, 4-bromo-9-chloro-7-ethylsulfanyl-2,5-dimethyl-pyrazolo[4,3-f]quinazoline. A solution of 4-bromo-7-ethylsulfanyl-2,5-dimethyl-pyrazolo[4,3-f]quinazolin-9-ol (1352 mg, 3.83 mmol) and phosphorus oxychloride (16.05 mL, 172.23 mmol) in DMF (19.2 mL) was stirred at 90° C. for 1 hr. The mixture was cooled down and then poured into ice cold aqueous saturated NaHCO₃, extracted in DCM three times, washed with brine, dried over magnesium sulfate, filtered and evaporated to give 4-bromo-9-chloro-7-ethylsulfanyl-2,5-dimethyl-pyrazolo[4,3-f]quinazoline (920 mg, 2.4752 mmol, 65% yield) as a brown powder. Material was used without further purification.

UPLC-MS (ES⁺, Method 5): 2.34 min, m/z 372.9 [M+H]⁺.

Step E, 4-bromo-9-chloro-7-ethylsulfonyl-2,5-dimethyl-pyrazolo[4,3-f]quinazoline (1-96). To a solution of 4-bromo-9-chloro-7-ethylsulfanyl-2,5-dimethyl-pyrazolo[4,3-f]quinazoline (1420. mg, 3.82 mmol) in DCM (38.3 mL) under N₂ was added m-chloroperbenzoic acid, MCPBA (2175.7 mg, 12.6 mmol). The reaction was stirred at 25° C. for 1 hr. The mixture was diluted with DCM and washed with aqueous saturated NaHCO₃ and brine. The organics were dried over anhydrous Na₂SO₄, filtered and concentrated to give product 4-bromo-9-chloro-7-ethylsulfonyl-2,5-dimethyl-pyrazolo[4,3-f]quinazoline (900 mg, 2.23 mmol, 58% yield) as a yellow solid. Material was used without further purification.

UPLC-MS (ES⁺, Method 5): 1.77 min, m/z 404.9 [M+H]⁺.

The intermediates in table 6 below were prepared by analogy with I-96 and 196a (intermediate scheme 33), replacing with building block ethyl N-[(7-bromo-2,6-dimethyl-indazol-5-yl)carbamothioyl]carbamate as described in the table entry.

TABLE 6

| Intermediate | | |
|---|---|---|
| Building block | Intermediate Structure/Name | UPLC-MS (ES⁺): min, m/z |
| 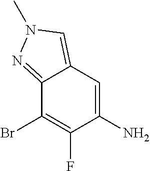 I-52a | Intermediate I-117a 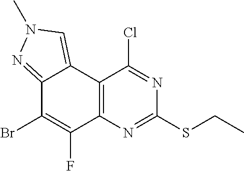 [SMILES: CCSc1nc(Cl)c2c(n1)c(F)c(Br)c1nn(C)cc12] | 2.23 min, 374.9/376.9/378.8 [M + H]⁺ (Method 5) |
| 7-bromo-6-fluoro-2-methyl- | I-117a | |
| indazol-5-amine (I-52a) | 4-bromo-9-chloro-7- | |
| | ethylsulfanyl-5-fluoro-2- | |
| | methyl-pyrazolo[4,3- | |
| | f]quinazoline | |
| 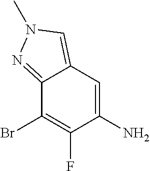 I-52a | Intermediate I-117 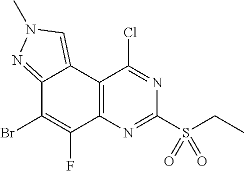 [SMILES: CCS(=O)(=O)c1nc(Cl)c2c(n1)c(F)c(Br)c1nn(C)cc12] | 1.68 min, 406.9/408.9 [M + H]⁺ (Method 5) |
| 7-bromo-6-fluoro-2-methyl- | I-117 | |
| indazol-5-amine (I-52a) | 4-bromo-9-chloro-7- | |
| | ethylsulfonyl-5-fluoro-2- | |
| | methyl-pyrazolo[4,3- | |
| | f]quinazoline | |
| 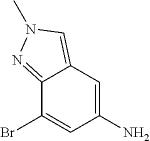 7-bromo-2-methyl- | Intermediate I-124 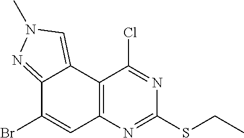 [SMILES: CCSc1nc(Cl)c2c(cc(Br)c3nn(C)cc32)n1] | 2.23 min, 356.9/358.9/360.8 [M + H]⁺ (Method 2) |
| indazol-5-amine | I-124a | |
| 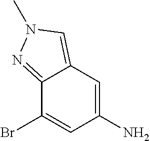 7-bromo-2-methyl- indazol-5-amine | Intermediate I-124a 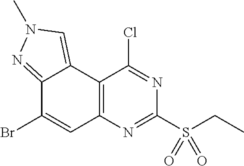 [SMILES: CCS(=O)(=O)c1nc(Cl)c2c(cc(Br)c3nn(C)cc32)n1] | 1.52 min, 389.9/390.9/392.9 [M + H]⁺ (Method 2) |
| | I-124 | |

### Intermediate I-138, 4-[8-ethyl-7-fluoro-3-(methoxymethoxy)-1-naphthyl]-2-methyl-7-[[(2R,8S)-2-fluoro-1,2,3,5,6,7-hexahydropyrrolizin-8-yl]methoxy]pyrazolo[4,3-f]quinazolin-9-ol

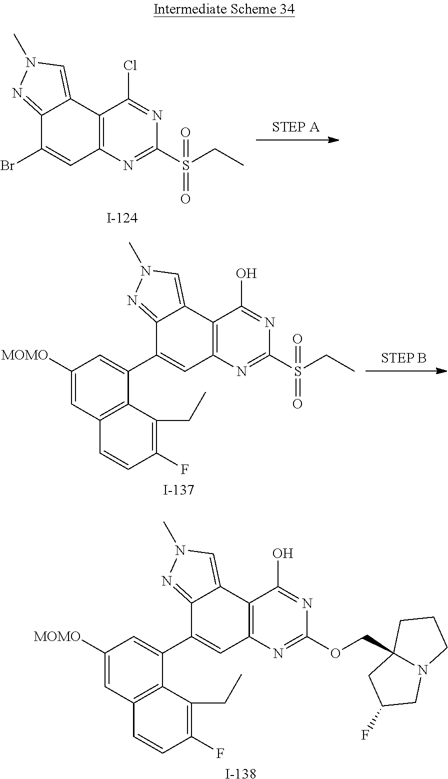

Step A, 4-[8-ethyl-7-fluoro-3-(methoxymethoxy)-1-naphthyl]-7-ethylsulfonyl-2-methyl-pyrazolo[4,3-f]quinazolin-9-ol. A suspension of 4-bromo-9-chloro-7-ethylsulfonyl-2-methyl-pyrazolo[4,3-f]quinazoline (1.51 g, 3.88 mmol), 2-[8-ethyl-7-fluoro-3-(methoxymethoxy)-1-naphthyl]-4,4,5,5-tetramethyl-1,3,2-dioxaborolane (1.82 g, 5.04 mmol) and Cesium Carbonate (2.53 g, 7.77 mmol) in 1,4-Dioxane (32 mL)/Water (6.5 mL) was degassed with N₂ for 5 minutes then [1,1′-Bis(di-tert-butylphosphino)ferrocene]dichloropalladium(II) (379 mg, 0.58 mmol) was added and the mixture was heated at 100° C. for 90 minutes. The mixture was filtered through a plug of celite and the filter cake washed with methanol. The filtrate was concentrated and the residue purified by flash chromatography (40 g column, eluting int 0-20% [NH₃ 1N in methanol] in DCM). Like fractions were pooled and concentrated. The residue was suspended in petroleum ether, filtered and dried to afford 4-[8-ethyl-7-fluoro-3-(methoxymethoxy)-1-naphthyl]-7-ethylsulfonyl-2-methyl-pyrazolo[4,3-f]quinazolin-9-ol (1.372 g, 2.6155 mmol, 67% yield) as a brown solid.

UPLC-MS (ES⁺, Method 5): 1.99 min, m/z 525.1 [M+H]⁺.

Step B, 4-[8-ethyl-7-fluoro-3-(methoxymethoxy)-1-naphthyl]-2-methyl-7-[(2R,8S)-2-fluoro-1,2,3,5,6,7-hexahydropyrrolizin-8-yl]methoxy]pyrazolo[4,3-f]quinazolin-9-ol (I-138). To a solution of 4-[8-ethyl-7-fluoro-3-(methoxymethoxy)-1-naphthyl]-7-ethylsulfonyl-2-methyl-pyrazolo[4,3-f]quinazolin-9-ol (1.07 g, 2.03 mmol) and ((2R,7aS)-2-Fluorohexahydro-1H-pyrrolizin-7a-yl) methanol (405. mg, 2.54 mmol) in anhydrous THF (6.7 mL) at 0° C., under N₂, was slowly added sodium tert-pentoxide 30% in 2-Methyl THF (2.05 mL, 5.08 mmol). The mixture was warmed to room temperature and left to stir for 2 hours. The mixture was cooled to 0° C. and water (30 mL) was slowly added. The mixture was extracted with EtOAc (4×15 mL). The combined organic layers were pooled, passed through a phase separator cartridge and concentrated. The residue was purified by flash chromatography (25 g column, eluting in 0-5% MeOH in DCM). Like fractions were pooled and concentrated to afford 4-[8-ethyl-7-fluoro-3-(methoxymethoxy)-1-naphthyl]-2-methyl-7-[(2R,8S)-2-fluoro-1,2,3,5,6,7-hexahydropyrrolizin-8-yl]methoxy]pyrazolo[4,3-f]quinazolin-9-ol (935 mg, 1.5857 mmol, 78% yield) as a light beige solid.

UPLC-MS (ES⁺, Method 5): 1.68 min, m/z 590.2 [M+H]⁺.

### Intermediate 1-58, 4-(4-bromo-7-chloro-1-tetrahydropyran-2-yl-pyrazolo[3,4-f]quinazolin-9-yl)-1,4-oxazepane

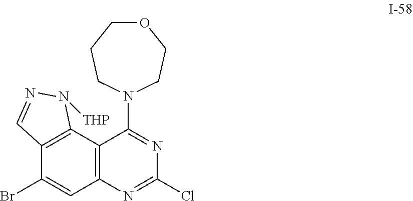

Intermediate 1-58 was prepared following the route in Intermediate Scheme 35 below.

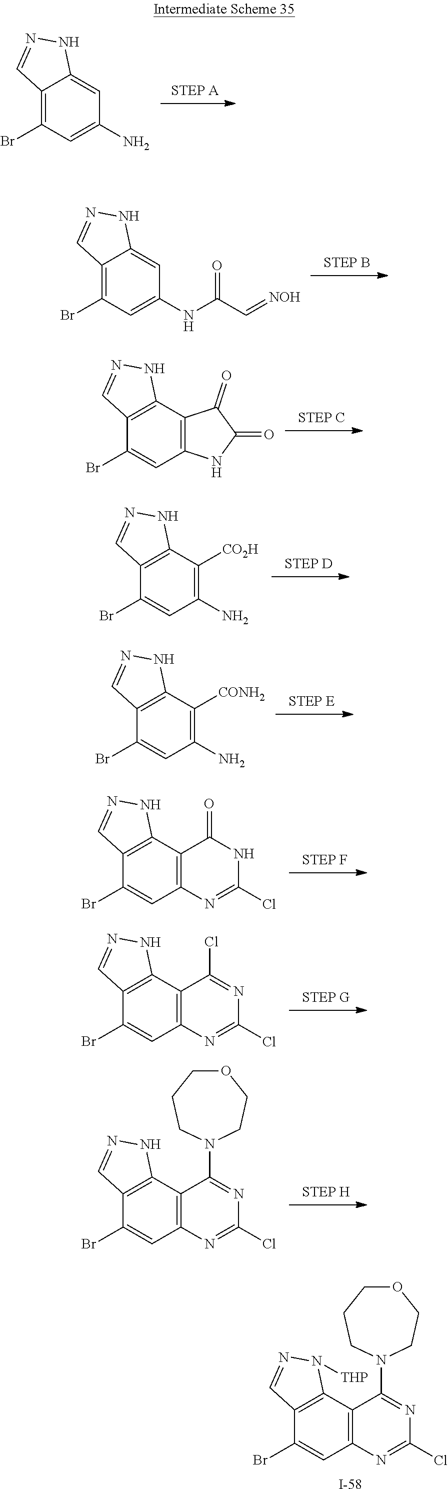

Step A, (2E)-N-(4-bromo-1H-indazol-6-yl)-2-hydroxyimino-acetamide. To a suspension of Chloral hydrate (2.39 g, 14.56 mmol), 4-bromo-1H-indazol-6-amine (1544. mg, 7.28 mmol) and sodium sulfate (8.27 g, 58.25 mmol) in a mixture of DMF (11.558 mL) and water (24.849 mL) was added hydrogen chloride (3.29 mL, 40.05 mmol) (conc HCl). The reaction mixture was heated to 90° C. and left to stir for 1 hour. Hydroxylamine hydrochloride (1.77 g, 25.48 mmol) was added and the reaction mixture left to stir at 90° C. for a further 30 mins. The reaction mixture was allowed to cool to room temperature, water (100 mL) added and the solid precipitate collected by vacuum filtration. The solid precipitate was washed with water (2×10 mL) and dried under vacuum to afford (2E)-N-(4-bromo-1H-indazol-6-yl)-2-hydroxyimino-acetamide (1973.6 mg, 6.9719 mmol, 96% yield) as a brown solid.

UPLC-MS (ES⁺, Method 2): 1.29 min, m/z 282.9/284.9 [M+H]⁺.

Step B, 4-bromo-1,6-dihydropyrrolo[2,3-g]indazole-7,8-dione. A stirring mixture of (2E)-N-(4-bromo-1H-indazol-6-yl)-2-hydroxyimino-acetamide (1973.6 mg, 6.97 mmol) and sulfuric acid (13.01 mL, 244.02 mmol) was heated to 60° C. and left to stir for 1 hour. The reaction mixture was allowed to cool room temperature, Ice-water (100 mL) added and the mixture left to stir for 5 min. The solid precipitate was collected by vacuum filtration, washed with water (2×10 mL) and dried under vacuum to afford 4-bromo-1,6-dihydropyrrolo[2,3-g]indazole-7,8-dione (1563.9 mg, 5.8782 mmol, 84% yield) as a brown solid.

UPLC-MS (ES⁺, Method 2): 1.20 min, m/z 265.9/267.9 [M+H]⁺.

Step C, 6-amino-4-bromo-1H-indazole-7-carboxylic acid. To an ice-cooled suspension of 4-bromo-1,6-dihydropyrrolo[2,3-g]indazole-7,8-dione (1563.9 mg, 5.88 mmol) in 1,4-dioxane (26.592 mL) was added a solution of sodium hydroxide (940.45 mg, 23.51 mmol) in water (2.799 mL). Then hydrogen peroxide solution 30 wt % in H₂O (1.2 mL, 11.76 mmol) was added. The reaction mixture was allowed to warm to room temperature and left to stir for 20 min. The reaction mixture was quenched with a saturated solution of sodium thiosulphate, then acidified to ~pH2 with 2M HCl (aq) and the solid precipitate collected by vacuum filtration. Solid precipitate was washed with water (2×10 mL) and dried under vacuum to afford 6-amino-4-bromo-1H-indazole-7-carboxylic acid (1503.8 mg, 5.8728 mmol, 100% yield) as a brown solid.

UPLC-MS (ES⁺, Method 2): 1.29 min, m/z 255.9/257.9 [M+H]⁺.

Step D, 6-amino-4-bromo-1H-indazole-7-carboxamide. HATU (2456.33 mg, 6.46 mmol) was added to a mixture of 6-amino-4-bromo-1H-indazole-7-carboxylic acid (1503.8 mg, 5.87 mmol), ammonium chloride (3141.47 mg, 58.73 mmol) and N,N-Diisopropylethylamine (3.07 mL, 17.62 mmol) in DMF (29.364 mL). The reaction mixture was left to stir at room temperature for 1 hour. Additional HATU (446.61 mg, 1.17 mmol) was added and the mixture left to stir for a further 30 mins. The reaction mixture was suspended in water (100 mL) and the solid precipitate collected by vacuum filtration. Ethyl acetate (100 mL) was added and both layers partitioned. Aqueous layer extracted with ethyl acetate (50 ml). Organic layers combined, washed with water (50 mL), saturated solution of brine (50 mL), dried over sodium sulfate, filtered, combined with the solid precipitate and concentrated under reduced pressure to afford 6-amino-4-bromo-1H-indazole-7-carboxamide (1498 mg, 5.87 mmol, 100% yield) as a brown solid.

UPLC-MS (ES⁺, Method 2): 1.21 min, m/z 254.9/256.9 [M+H]⁺.

Step E, 4-bromo-7-chloro-1,8-dihydropyrazolo[3,4-f]quinazolin-9-one. To a suspension of 6-amino-4-bromo-1H-indazole-7-carboxamide (1498. mg, 5.87 mmol) in 1,4-Dioxane (23.492 mL) was added thiophosgene (0.95 mL, 12.33 mmol). The reaction mixture was heated to 105° C. and left to stir for 1 hour. The reaction mixture was allowed to cool to room temperature and concentrated under vacuum. The crude residue was triturated with petrol (10 mL) and dried under vacuum. The residue was again suspended in 1,4-Dioxane (23.492 mL) and thiophosgene (0.95 mL, 12.33 mmol) added. The reaction mixture was heated to 105° C. and left to stir for 30 mins. The reaction mixture was allowed to cool to room temperature and concentrated under vacuum. The crude residue was triturated with petrol (10 mL) and dried under vacuum to afford 4-bromo-7-chloro-1,8-dihydropyrazolo[3,4-f]quinazolin-9-one (1759 mg, 5.8729 mmol, 100% yield) as a brown solid.

UPLC-MS (ES⁺, Method 2): 1.42 min, m/z 300.9 [M+H]⁺.

Step F, 4-bromo-7,9-dichloro-1H-pyrazolo[3,4-f]quinazoline. Phosphorus oxychloride (8.21 mL, 88.09 mmol) was added to a suspension of 4-bromo-7-chloro-1,8-dihydropyrazolo[3,4-f]quinazolin-9-one (1759. mg, 5.87 mmol) and N,N-Diisopropylethylamine (3.07 mL, 17.62 mmol). The reaction mixture was heated to 90° C. and left to stir for 30 min. The mixture was allowed to cool to room temperature and quenched carefully into water. The solid precipitate was collected by vacuum filtration and washed with water (2×20 mL). Ethyl acetate (50 mL) was added to the filtrate and the organic layer separated. The aqueous layer extracted with ethyl acetate (50 mL) and organic layers combined. Combined organic layers were washed with a saturated solution of brine (10 mL), dried over sodium sulfate, filtered, combined with the solid precipitate and concentrated under vacuum to afford 4-bromo-7,9-dichloro-1H-pyrazolo[3,4-f]quinazoline (1544.1 mg, 4.8564 mmol, 83% yield) as a brown solid.

UPLC-MS (ES⁺, Method 2): 1.75 min, m/z 318.8 [M+H]⁺.

Step G, 4-(4-bromo-7-chloro-1H-pyrazolo[3,4-f]quinazolin-9-yl)-1,4-oxazepane. At 0° C., N, N-Diisopropylethylamine (2.54 mL, 14.57 mmol) was added to a solution of 1,4-Oxazepane hydrochloride (835.37 mg, 6.07 mmol) and 4-bromo-7,9-dichloro-1H-pyrazolo[3,4-f]quinazoline (1544.1 mg, 4.86 mmol) in DCM (19.426 mL). The reaction mixture was left to stir at room temperature for 30 min. The reaction mixture was concentrated under vacuum and purified by flash column chromatography on silica gel eluting with 0-8% MeOH in DCM to afford 4-(4-bromo-7-chloro-1H-pyrazolo[3,4-f]quinazolin-9-yl)-1,4-oxazepane (702.9 mg, 1.8369 mmol, 38% yield) as a brown solid. 1.66 min, m/z 383.9

UPLC-MS (ES⁺, Method 2): 1.66 min, m/z 382.0 [M+H]⁺.

Step H, 4-(4-bromo-7-chloro-1-tetrahydropyran-2-yl-pyrazolo[3,4-f]quinazolin-9-yl)-1,4-oxazepane (1-58). A suspension of 3,4-Dihydro-2H-pyran (0.12 mL, 1.31 mmol), p-toluenesulfonic acid monohydrate (19.88 mg, 0.1 mmol) and 4-(4-bromo-7-chloro-1H-pyrazolo[3,4-f]quinazolin-9-yl)-1,4-oxazepane (200. mg, 0.52 mmol) in THF (2.1 mL) was allowed to stir at room temperature overnight. Additional 3,4-Dihydro-2H-pyran (0.12 mL, 1.31 mmol) and p-toluenesulfonic acid monohydrate (19.88 mg, 0.1 mmol) were added and the reaction mixture was again left to stir at room temperature overnight. The reaction mixture was concentrated under vacuum and purified by flash column chromatography on silica gel eluting with 0-50% ethyl acetate in petrol to afford 4-(4-bromo-7-chloro-1-tetrahydropyran-2-yl-pyrazolo[3,4-f]quinazolin-9-yl)-1,4-oxazepane (118.6 mg, 0.2541 mmol, 49% yield) as a yellow oil.

UPLC-MS (ES⁺, Method 2): 2.04 min, m/z 468.0 [M+H]⁺.

### Intermediate 1-2, 4-(4-bromo-7-chloro-2-methyl-pyrazolo[4,3-f]quinazolin-9-yl)-1,4-oxazepane

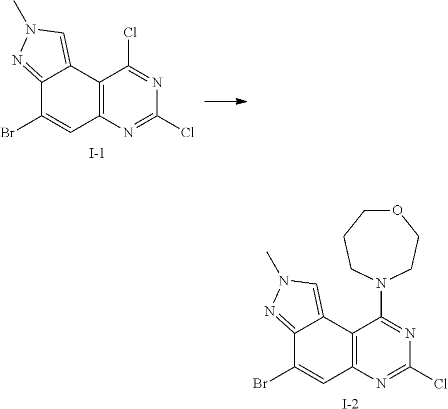

### Intermediate Scheme 36

4-(4-bromo-7-chloro-2-methyl-pyrazolo[4,3-f]quinazolin-9-yl)-1,4-oxazepane. To a solution of 4-bromo-7,9-dichloro-2-methyl-pyrazolo[4,3-f]quinazoline (112 mg, 0.34 mmol) and N,N-diisopropylethylamine (0.59 mL, 3.37 mmol) in THF (2.2 mL) was added 1,4-oxazepane hydrochloride (51.1 mg, 0.37 mmol) and stirred at room temperature for 1 hour. Further 1,4-oxazepane hydrochloride (51.07 mg, 0.37 mmol) was added and stirring continued for a further 5 hours. The solvent was removed in vacuo, and the residue loaded onto silica with DCM. The crude was purified by flash column chromatography (12 g SiO₂, 5-100% EtOAc in petroleum ether). The desired fractions were concentrated to afford 4-(4-bromo-7-chloro-2-methyl-pyrazolo[4,3-f]quinazolin-9-yl)-1,4-oxazepane (I-2) (50 mg, 0.126 mmol, 37% yield) as a yellow solid.

UPLC-MS (ES⁺, Method 2): 1.73 min, m/z 398.0, 400.0 [M+H]⁺.

The intermediates in table 7 below were prepared by analogy with I-2 (intermediate scheme 36), replacing intermediate 1-1 and building block 1,4-oxazepane hydrochloride as described in the table entry.

TABLE 7

| Intermediate Building | | |
|---|---|---|
| block | Intermediate Structure/Name | UPLC-MS (ES⁺): min, m/z |
| Intermediate I-1 and | Intermediate I-5 | 1.16 min, 410.0/412.0 |
| 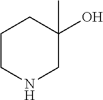 3-Methylpiperidin-3-ol | 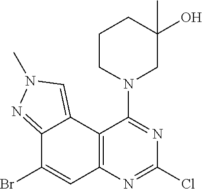 [SMILES: Cn1cc2c(n1)c(Br)cc1nc(Cl)nc(N3CCCC(C)(O)C3)c12] | [M + H]⁺ (Method 4) |
| | I-5 | |
| | 1-(4-bromo-7-chloro-2-methyl- | |
| | pyrazolo[4,3-f]quinazolin-9-yl)-3- | |
| | methyl-piperidin-3-ol | |
| Intermediate I-1 and | Intermediate I-7 | 1.49 min, 426.0/428.0 |
| 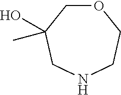 6-Methyl-1,4-oxazepan-6-ol •HCl | 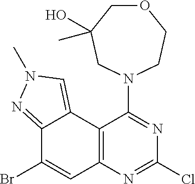 [SMILES: Cn1cc2c(n1)c(Br)cc1nc(Cl)nc(N3CCOCC(C)(O)C3)c12] | [M + H]⁺ (Method 4) |
| | I-7 | |
| | 4-(4-bromo-7-chloro-2-methyl- | |
| | pyrazolo[4,3-f]quinazolin-9-yl)-6- | |
| | methyl-1,4-oxazepan-6-ol | |
| Intermediate I-1 and | Intermediate I-9 | 1.73 min, 393.2/395.2 |
| 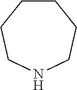 Azepane | 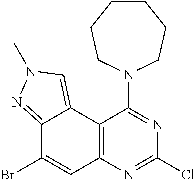 [SMILES: Cn1cc2c(n1)c(Br)cc1nc(Cl)nc(N3CCCCCC3)c12] | [M + H]⁺ (Method 4) |
| | I-9 | |
| | 9-(azepan-1-yl)-4-bromo-7- | |
| | chloro-2-methyl-pyrazolo[4,3- | |
| | f]quinazoline | |
| | 4-(4-bromo-7-chloro-2-methyl- | |
| | pyrazolo[4,3-f]quinazolin-9-yl)-6- | |
| | methyl-1,4-oxazepan-6-ol | |
| Intermediate I-1 and | Intermediate I-11 | 1.67 min, 426.0/428.0 |
| 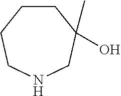 3-methylazepan-3-ol | 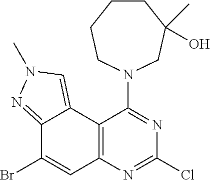 [SMILES: Cn1cc2c(n1)c(Br)cc1nc(Cl)nc(N3CCCCC(C)(O)C3)c12] | [M + H]⁺ (Method 4) |
| | I-11 | |
| | 1-(4-bromo-7-chloro-2-methyl- | |
| | pyrazolo[4,3-f]quinazolin-9-yl)-3- | |
| | methyl-azepan-3-ol | |
| Intermediate I-1 and | Intermediate I-13 | 2.72 min, 481.1/483.1 |
| 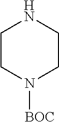 [SMILES: CC(C)(C)OC(=O)N1CCNCC1] N-boc-piperazine | 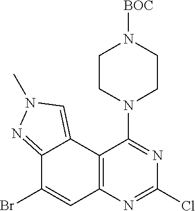 [SMILES: Cn1cc2c(n1)c(Br)cc1nc(Cl)nc(N3CCN(C(=O)OC(C)(C)C)CC3)c12] | [M + H]⁺ (Method 3) |
| | I-13 | |
| | tert-butyl 4-(4-bromo-7-chloro-2- | |
| | methyl-pyrazolo[4,3-f]quinazolin- | |
| | 9-yl)piperazine-1-carboxylate | |
| Intermediate I-1 and | Intermediate I-15 | 2.73 min, 505.1/507.1 |
| 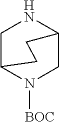 [SMILES: CC(C)(C)OC(=O)N1CC2CCC1CN2] tert-butyl 2,5- diazabicyclo[2.2.2]octane- 2-carboxylate | 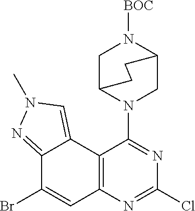 [SMILES: Cn1cc2c(n1)c(Br)cc1nc(Cl)nc(N3CC4CCC3CN4C(=O)OC(C)(C)C)c12] | [M + H]⁺ (Method 4) |
| | I-15 | |
| | tert-butyl 5-(4-bromo-7-chloro-2- | |
| | methyl-pyrazolo[4,3-f]quinazolin- | |
| | 9-yl)-2,5- | |
| | diazabicyclo[2.2.2]octane-2- | |
| | carboxylate | |
| Intermediate I-1 and | Intermediate I-17 | 2.84 min, 505.1/507.1 |
| 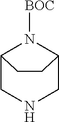 [SMILES: CC(C)(C)OC(=O)N1C2CCC1CNC2] 8-boc-3,8- diazabicyclo[3.2.1]octane | 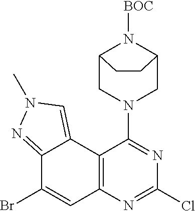 [SMILES: Cn1cc2c(n1)c(Br)cc1nc(Cl)nc(N3CC4CCC(C3)N4C(=O)OC(C)(C)C)c12] | [M + H]⁺ (Method 3) |
| | I-17 | |
| | tert-butyl 3-(4-bromo-7-chloro-2- | |
| | methyl-pyrazolo[4,3-f]quinazolin- | |
| | 9-yl)-3,8- | |
| | diazabicyclo[3.2.1]octane-8- | |
| | carboxylate | |
| Intermediate I-1 and | Intermediate I-19 | 1.28 min, 437.0/439.0 |
| 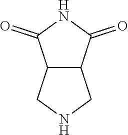 2,3,3a,6a-tetrahydro-1H- pyrrolo[3,4-c]pyrrole-4,6- dione | 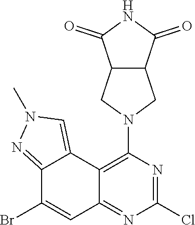 [SMILES: Cn1cc2c(n1)c(Br)cc1nc(Cl)nc(N3CC4C(=O)NC(=O)C4C3)c12] | [M + H]⁺ (Method 4) |
| | I-19 | |
| | 5-(4-bromo-7-chloro-2-methyl- | |
| | pyrazolo[4,3-f]quinazolin-9-yl)- | |
| | 3a,4,6,6a-tetrahydropyrrolo[3,4- | |
| | c]pyrrole-1,3-dione | |
| Intermediate I-1 and | Intermediate I-23 | 2.93 min, 495.3 [M + H]⁺ |
| 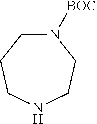 [SMILES: CC(C)(C)OC(=O)N1CCCNCC1] tert-butyl 1,4-diazepane- 1-carboxylate | 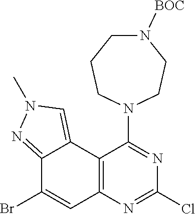 [SMILES: Cn1cc2c(n1)c(Br)cc1nc(Cl)nc(N3CCCN(C(=O)OC(C)(C)C)CC3)c12] | (Method 4) |
| | I-23 | |
| | tert-butyl 4-(4-bromo-7-chloro-2- | |
| | methyl-pyrazolo[4,3-f]quinazolin- | |
| | 9-yl)-1,4-diazepane-1- | |
| | carboxylate | |
| Intermediate I-1 and | Intermediate I-25 | 1.60 min, 505.0 [M + H]⁺ |
| 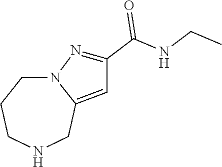 N-ethyl-5,6,7,8- tetrahydro-4H-pyrazolo[1,5- a][1,4]diazepine-2- carboxamide (A-6) | 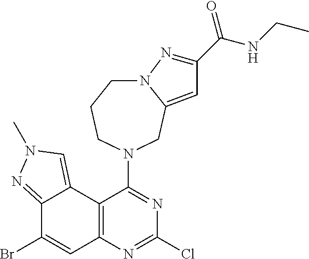 [SMILES: CCNC(=O)c1cc2n(n1)CCCN(c1nc(Cl)nc3cc(Br)c4nn(C)cc4c13)C2] | (Method 4) |
| | I-25 | |
| | 5-(4-bromo-7-chloro-2-methyl- | |
| | pyrazolo[4,3-f]quinazolin-9-yl)- | |
| | N-ethyl-4,6,7,8- | |
| | tetrahydropyrazolo[1,5- | |
| | a][1,4]diazepine-2-carboxamide | |
| Intermediate I-1 and | Intermediate I-27 | 1.57 min, 505.0 [M + H]⁺ |
| 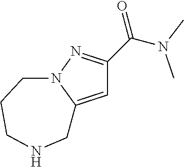 N,N-dimethyl-5,6,7,8- tetrahydro-4H-pyrazolo[1,5- a][1,4]diazepine-2- carboxamide (A-7) | 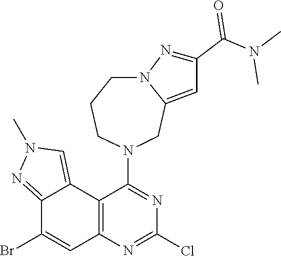 [SMILES: CN(C)C(=O)c1cc2n(n1)CCCN(c1nc(Cl)nc3cc(Br)c4nn(C)cc4c13)C2] | (Method 4) |
| | I-27 | |
| | 5-(4-bromo-7-chloro-2-methyl- | |
| | pyrazolo[4,3-f]quinazolin-9-yl)- | |
| | N,N-dimethyl-4,6,7,8- | |
| | tetrahydropyrazolo[1,5- | |
| | a][1,4]diazepine-2-carboxamide | |
| Intermediate I-1 and | Intermediate I-29 | 1.85 min, 529.1 [M + H]⁺ |
| 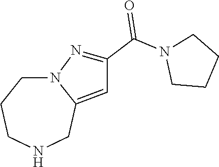 [SMILES: O=C(c1cc2n(n1)CCCNC2)N1CCCC1] pyrrolidin-1-yl(5,6,7,8- tetrahydro-4H-pyrazolo[1,5- a][1,4]diazepin-2- yl)methanone (A-8) | 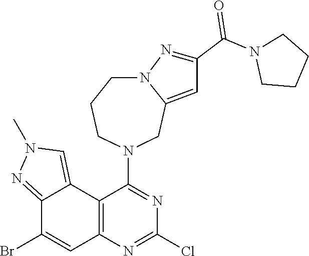 [SMILES: Cn1cc2c(n1)c(Br)cc1nc(Cl)nc(N3CCCn4nc(C(=O)N5CCCC5)cc4C3)c12] | (Method 4) |
| | I-29 | |
| | [5-(4-bromo-7-chloro-2-methyl- | |
| | pyrazolo[4,3-f]quinazolin-9-yl)- | |
| | 4,6,7,8-tetrahydropyrazolo[1,5- | |
| | a][1,4]diazepin-2-yl]-pyrrolidin-1- | |
| | yl-methanone | |
| Intermediate I-1 and | Intermediate I-31 | 1.58 min, 545.1/547.0 |
| 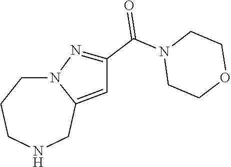 [SMILES: O=C(c1cc2n(n1)CCCNC2)N1CCOCC1] morpholino(5,6,7,8- tetrahydro-4H-pyrazolo[1,5- a][1,4]diazepin-2- yl)methanone (A-9) | 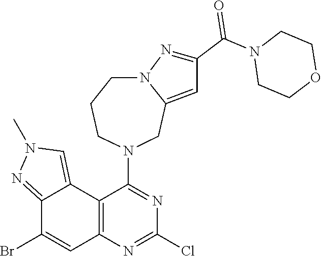 [SMILES: Cn1cc2c(n1)c(Br)cc1nc(Cl)nc(N3CCCn4nc(C(=O)N5CCOCC5)cc4C3)c12] | [M + H]⁺ (Method 4) |
| | I-31 | |
| | [5-(4-bromo-7-chloro-2-methyl- | |
| | pyrazolo[4,3-f]quinazolin-9-yl)- | |
| | 4,6,7,8-tetrahydropyrazolo[1,5- | |
| | a][1,4]diazepin-2-yl]-morpholino- | |
| | methanone | |
| Intermediate I-1 and | Intermediate I-33 | 2.54 min, 507/509/511 |
| 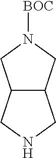 [SMILES: CC(C)(C)OC(=O)N1CC2CNCC2C1] tert-Butyl hexahydropyrrolo[3,4- c]pyrrole-2(1H)- carboxylate | 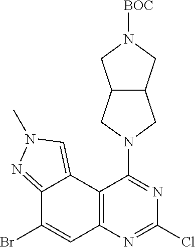 [SMILES: Cn1cc2c(n1)c(Br)cc1nc(Cl)nc(N3CC4CN(C(=O)OC(C)(C)C)CC4C3)c12] | [M + H]⁺ (Method 4) |
| | I-33 | |
| | tert-butyl 2-(4-bromo-7-chloro-2- | |
| | methyl-pyrazolo[4,3-f]quinazolin- | |
| | 9-yl)-1,3,3a,4,6,6a- | |
| | hexahydropyrrolo[3,4-c]pyrrole- | |
| | 5-carboxylate | |
| Intermediate I-1 and | Intermediate I-36 | 1.67 min, 426.0 [M + H]⁺ |
| 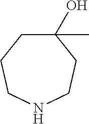 4-Hydroxy-4-Methyl- Hexahydro-1H-azepine hydrochloride | 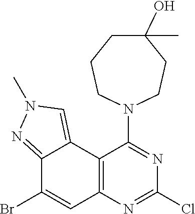 [SMILES: Cn1cc2c(n1)c(Br)cc1nc(Cl)nc(N3CCCC(C)(O)CC3)c12] | (Method 4) |
| | I-36 | |
| | 1-{4-bromo-7-chloro-2-methyl- | |
| | 2H-pyrazolo[4,3-f]quinazolin-9- | |
| | yl}-4-methylazepan-4-ol | |
| Intermediate I-1 and | Intermediate I-47 | 1.55 min, 412.0 [M + H]⁺ |
| 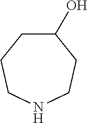 azepan-4-ol hydrochloride | 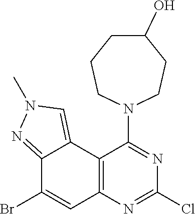 [SMILES: Cn1cc2c(n1)c(Br)cc1nc(Cl)nc(N3CCCC(O)CC3)c12] | (Method 4) |
| | I-47 | |
| | 1-(4-bromo-7-chloro-2-methyl- | |
| | pyrazolo[4,3-f]quinazolin-9- | |
| | yl)azepan-4-ol | |
| Intermediate I-49 and | Intermediate I-50 | 2.33 min, 422.0/423.9 |
| 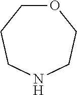 1,4-Oxazepane hydrochloride | 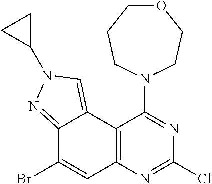 [SMILES: Clc1nc(N2CCCOCC2)c2c(cc(Br)c3nn(C4CC4)cc32)n1] | [M + H]⁺ (Method 4) |
| | I-50 | |
| | 4-(4-bromo-7-chloro-2- | |
| | cyclopropyl-pyrazolo[4,3- | |
| | f]quinazolin-9-yl)-1,4-oxazepane | |
| Intermediate I-52 and | Intermediate I-53 | 1.95 min, 413.0 [M + H]⁺ |
| 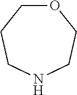 1,4-Oxazepane hydrochloride | 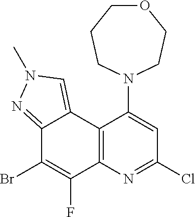 [SMILES: Cn1cc2c(n1)c(Br)c(F)c1nc(Cl)cc(N3CCCOCC3)c12] | (Method 4) |
| | I-53 | |
| | 4-(4-bromo-7-chloro-5-fluoro-2- | |
| | methyl-pyrazolo[4,3-f]quinolin-9- | |
| | yl)-1,4-oxazepane | |
| Intermediate I-55 and | Intermediate I-56 | 1.47 min, 395.9 [M + H]⁺ |
| 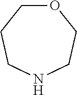 1,4-Oxazepane hydrochloride | 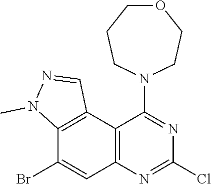 [SMILES: Cn1ncc2c3c(N4CCCOCC4)nc(Cl)nc3cc(Br)c21] | (Method 4) |
| | I-56 | |
| | 4-(4-bromo-7-chloro-3-methyl- | |
| | pyrazolo[4,3-f]quinazolin-9-yl)- | |
| | 1,4-oxazepane | |
| Intermediate I-1 and | Intermediate I-64 | 1.81 min, 434.0 [M + H]⁺ |
| 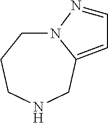 A-10 5,6,7,8-tetrahydro-4H- pyrazolo[1,5-a][1,4]diazepine | 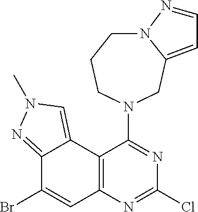 [SMILES: Cn1cc2c(n1)c(Br)cc1nc(Cl)nc(N3CCCn4nccc4C3)c12] | (Method 5) |
| | I-64 | |
| | 4-bromo-7-chloro-2-methyl-9- | |
| | (4,6,7,8-tetrahydropyrazolo[1,5- | |
| | a][1,4]diazepin-5- | |
| | yl)pyrazolo[4,3-f]quinazoline | |
| Intermediate I-1 and | Intermediate I-66 | 1.80 min, 446.0 [M + H]⁺ |
| 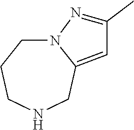 A-20 2-methyl-5,6,7,8- tetrahydro-4H-pyrazolo[1,5- a][1,4]diazepine (A-20) | 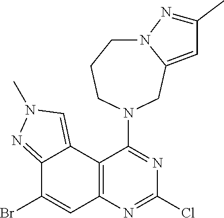 [SMILES: Cc1cc2n(n1)CCCN(c1nc(Cl)nc3cc(Br)c4nn(C)cc4c13)C2] | (Method 5) |
| | I-66 | |
| | 4-bromo-7-chloro-2-methyl-9-(2- | |
| | methyl-4,6,7,8- | |
| | tetrahydropyrazolo[1,5- | |
| | a][1,4]diazepin-5- | |
| | yl)pyrazolo[4,3-f]quinazoline | |
| Intermediate I-1 and | Intermediate I-68 | 1.87 min, 458.9 [M + H]⁺ |
| 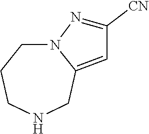 [SMILES: N#Cc1cc2n(n1)CCCNC2] A-21 5,6,7,8-tetrahydro-4H-pyrazolo[1,5- a][1,4]diazepine-2-carbonitrile | 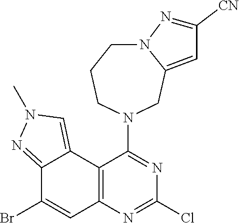 [SMILES: Cn1cc2c(n1)c(Br)cc1nc(Cl)nc(N3CCCn4nc(C#N)cc4C3)c12] | (Method 5) |
| | I-68 | |
| | 5-(4-bromo-7-chloro-2-methyl- | |
| | pyrazolo[4,3-f]quinazolin-9-yl)- | |
| | 4,6,7,8-tetrahydropyrazolo[1,5- | |
| | a][1,4]diazepine-2-carbonitrile | |
| Intermediate I-72 and | Intermediate I-73 | 1.74 min, 573.2 [M + H]⁺ |
| 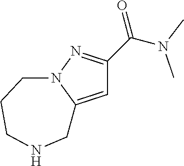 N,N-dimethyl-5,6,7,8- tetrahydro-4H-pyrazolo[1,5- a][1,4]diazepine-2- carboxamide (A-7) | 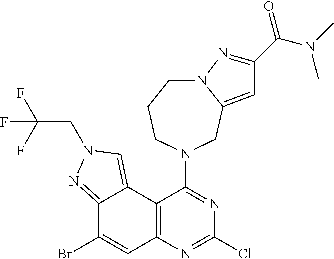 [SMILES: CN(C)C(=O)c1cc2n(n1)CCCN(c1nc(Cl)nc3cc(Br)c4nn(CC(F)(F)F)cc4c13)C2] | (Method 2) |
| | I-73 | |
| | 5-[4-bromo-7-chloro-2-(2,2,2- | |
| | trifluoroethyl)pyrazolo[4,3- | |
| | f]quinazolin-9-yl]-N,N-dimethyl- | |
| | 4,6,7,8-tetrahydropyrazolo[1,5- | |
| | a][1,4]diazepine-2-carboxamide | |
| Intermediate I-75 and | Intermediate I-76 | 2.01 min, 412.0 [M + H]⁺ |
| 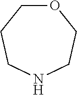 1,4-Oxazepane hydrochloride | 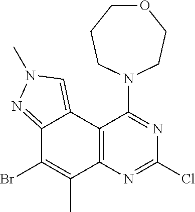 [SMILES: Cc1c(Br)c2nn(C)cc2c2c(N3CCCOCC3)nc(Cl)nc12] | (Method 5) |
| | I-76 | |
| | 4-(4-bromo-7-chloro-2,5- | |
| | dimethyl-pyrazolo[4,3- | |
| | f]quinazolin-9-yl)-1,4-oxazepane | |
| Intermediate I-75 and | Intermediate I-78 | 1.86 min, 519.0 [M + H]⁺ |
| 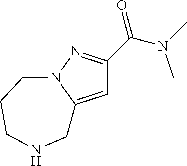 N,N-dimethyl-5,6,7,8- tetrahydro-4H- pyrazolo[1,5- a][1,4]diazepine-2- carboxamide (A-7) | 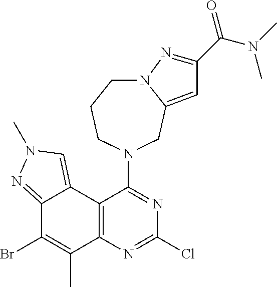 [SMILES: Cc1c(Br)c2nn(C)cc2c2c(N3CCCn4nc(C(=O)N(C)C)cc4C3)nc(Cl)nc12] | (Method 5) |
| | I-78 | |
| | 5-(4-bromo-7-chloro-2,5- | |
| | dimethyl-pyrazolo[4,3- | |
| | f]quinazolin-9-yl)-N,N-dimethyl- | |
| | 4,6,7,8-tetrahydropyrazolo[1,5- | |
| | a][1,4]diazepine-2-carboxamide | |
| Intermediate I-1 and | Intermediate I-81 | 2.22 min, 509.0 [M + H]⁺ |
| 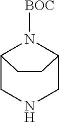 [SMILES: CC(C)(C)OC(=O)N1C2CCC1CNC2] 8-boc-3,8- diazabicyclo[3.2.1]octane | 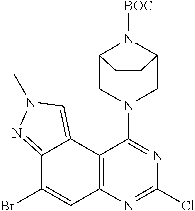 [SMILES: Cn1cc2c(n1)c(Br)cc1nc(Cl)nc(N3CC4CCC(C3)N4C(=O)OC(C)(C)C)c12] | (Method 5) |
| | I-81 | |
| | tert-butyl 3-(4-bromo-7-chloro-2- | |
| | methyl-pyrazolo[4,3-f]quinazolin- | |
| | 9-yl)-3,8- | |
| | diazabicyclo[3.2.1]octane-8- | |
| | carboxylate | |
| Intermediate I-52 and | Intermediate I-83 | 1.68 min, 523.0 [M + H]⁺ |
| 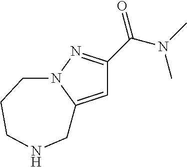 N,N-dimethyl-5,6,7,8- tetrahydro-4H- pyrazolo[1,5- a][1,4]diazepine-2- carboxamide (A-7) | 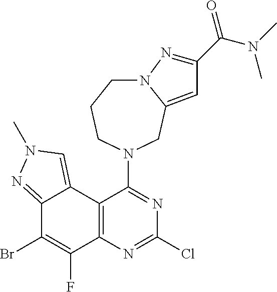 [SMILES: CN(C)C(=O)c1cc2n(n1)CCCN(c1nc(Cl)nc3c(F)c(Br)c4nn(C)cc4c13)C2] | (Method 5) |
| | I-83 | |
| | 5-(4-bromo-7-chloro-5-fluoro-2- | |
| | methyl-pyrazolo[4,3-f]quinazolin- | |
| | 9-yl)-N,N-dimethyl-4,6,7,8- | |
| | tetrahydropyrazolo[1,5- | |
| | a][1,4]diazepine-2-carboxamide | |
| Intermediate I-52 and | Intermediate I-85 | 1.79 min, 416.0 [M + H]⁺ |
| 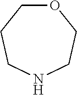 1,4-Oxazepane hydrochloride | 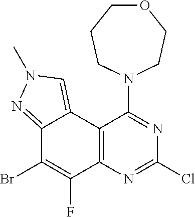 [SMILES: Cn1cc2c(n1)c(Br)c(F)c1nc(Cl)nc(N3CCCOCC3)c12] | (Method 5) |
| | I-85 | |
| | 4-(4-bromo-7-chloro-5-fluoro-2- | |
| | methyl-pyrazolo[4,3-f]quinazolin- | |
| | 9-yl)-1,4-oxazepane | |
| Intermediate I-52 and | Intermediate I-93 | 1.64 min, 445.9 [M + H]⁺ |
| 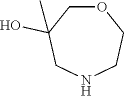 6-Methyl-1,4-oxazepan-6- ol hydrochloride | 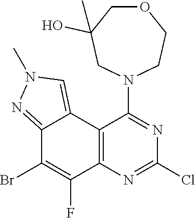 [SMILES: Cn1cc2c(n1)c(Br)c(F)c1nc(Cl)nc(N3CCOCC(C)(O)C3)c12] | (Method 5) |
| | I-93 | |
| | 4-(4-bromo-7-chloro-5-fluoro-2- | |
| | methyl-pyrazolo[4,3-f]quinazolin- | |
| | 9-yl)-6-methyl-1,4-oxazepan-6-ol | |
| Intermediate I-52 and | Intermediate I-103 | 2.18 min, 527.0 [M + H]⁺ |
| 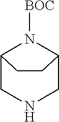 [SMILES: CC(C)(C)OC(=O)N1C2CCC1CNC2] 8-boc-3,8- diazabicyclo[3.2.1]octane | 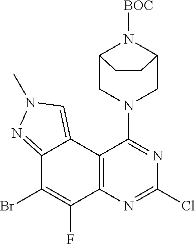 [SMILES: Cn1cc2c(n1)c(Br)c(F)c1nc(Cl)nc(N3CC4CCC(C3)N4C(=O)OC(C)(C)C)c12] | (Method 5) |
| | I-103 | |
| | tert-butyl 3-(4-bromo-7-chloro-5- | |
| | fluoro-2-methyl-pyrazolo[4,3- | |
| | f]quinazolin-9-yl)-3,8- | |
| | diazabicyclo[3.2.1]octane-8- | |
| | carboxylate | |
| Intermediate I-52 and | Intermediate I-105 | 1.79 min, 429.9 [M + H]⁺ |
| 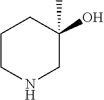 (R)-3-Methylpiperidin-3-ol hydrochloride | 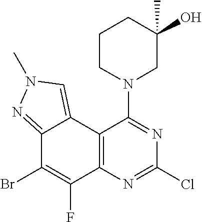 [SMILES: Cn1cc2c(n1)c(Br)c(F)c1nc(Cl)nc(N3CCC[C@@](C)(O)C3)c12] | (Method 5) |
| | I-105 | |
| | (3R)-1-(4-bromo-7-chloro-5- | |
| | fluoro-2-methyl-pyrazolo[4,3- | |
| | f]quinazolin-9-yl)-3-methyl- | |
| | piperidin-3-ol | |
| Intermediate I-1 and | Intermediate I-107 | 1.43 min, 491.0 [M + H]⁺ |
| 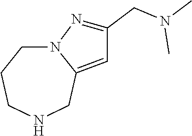 A-28 N,N-dimethyl-1-(5,6,7,8- tetrahydro-4H-pyrazolo[1,5- a][1,4]diazepin-2- | 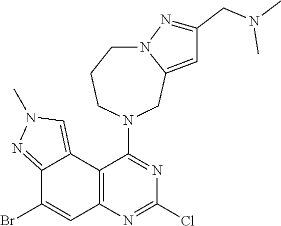 [SMILES: CN(C)Cc1cc2n(n1)CCCN(c1nc(Cl)nc3cc(Br)c4nn(C)cc4c13)C2] | (Method 5) |
| yl)methanamine (A-28) | I-107 | |
| | 1-[5-(4-bromo-7-chloro-2- | |
| | methyl-pyrazolo[4,3-f]quinazolin- | |
| | 9-yl)-4,6,7,8- | |
| | tetrahydropyrazolo[1,5- | |
| | a][1,4]diazepin-2-yl]-N,N- | |
| | dimethyl-methanamine | |
| Intermediate I-1 and | Intermediate I-109 | 1.71 min, 522.9 [M + H]⁺ |
| 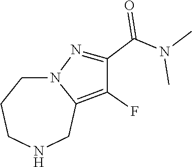 [SMILES: CN(C)C(=O)c1nn2c(c1F)CNCCC2] A-29 3-fluoro-N,N-dimethyl- 5,6,7,8-tetrahydro-4H-pyrazolo[1,5- a][1,4]diazepine-2- carboxamide (A-29) | 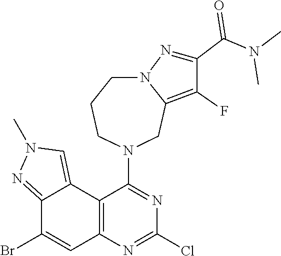 [SMILES: CN(C)C(=O)c1nn2c(c1F)CN(c1nc(Cl)nc3cc(Br)c4nn(C)cc4c13)CCC2] | (Method 5) |
| | I-109 | |
| | 5-(4-bromo-7-chloro-2-methyl- | |
| | pyrazolo[4,3-f]quinazolin-9-yl)-3- | |
| | fluoro-N,N-dimethyl-4,6,7,8- | |
| | tetrahydropyrazolo[1,5- | |
| | a][1,4]diazepine-2-carboxamide | |
| Intermediate I-52 and | Intermediate I-113 | 1.77 min, 411.9 [M + H]⁺ |
| 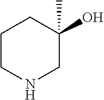 (R)-3-Methylpiperidin-3-ol hydrochloride | 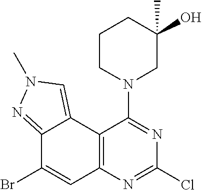 [SMILES: Cn1cc2c(n1)c(Br)cc1nc(Cl)nc(N3CCC[C@@](C)(O)C3)c12] | (Method 5) |
| | I-113 | |
| | (3R)-1-(4-bromo-7-chloro-2- | |
| | methyl-pyrazolo[4,3-f]quinazolin- | |
| | 9-yl)-3-methyl-piperidin-3-ol | |

### Intermediate 1-88, 4-bromo-7-chloro-9-[3-(3-methoxypropyl)-3,8-diazabicyclo[3.2.1]octan-8-yl]-2-methyl-pyrazolo[4,3-f]quinazoline

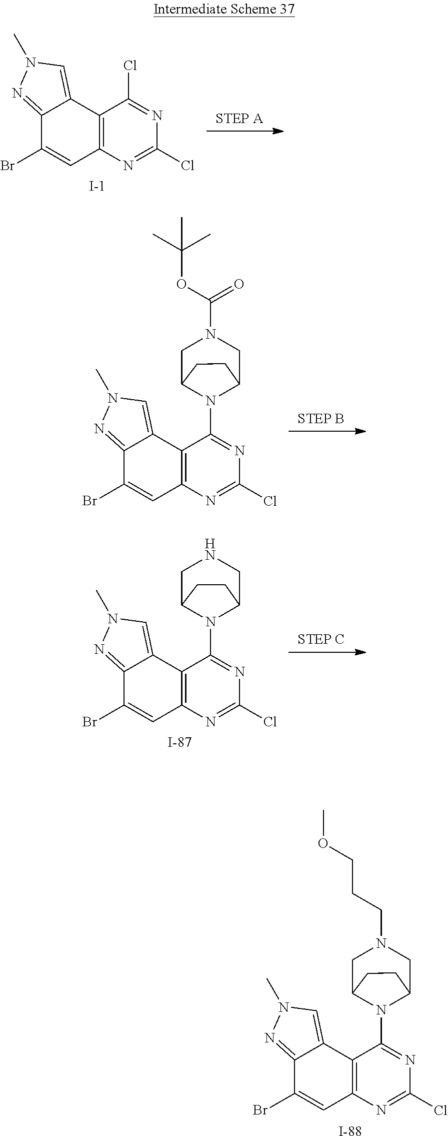

Step A, tert-butyl 8-(4-bromo-7-chloro-2-methyl-pyrazolo[4,3-f]quinazolin-9-yl)-3,8-diazabicyclo[3.2.1]octane-3-carboxylate. N, N-Diisopropylethylamine (0.39 mL, 2.26 mmol) was added to a stirring solution of 4-bromo-7,9-dichloro-2-methyl-pyrazolo[4,3-f]quinazoline (250 mg, (1-1) 0.75 mmol) and 3-Boc-3,8-diazabicyclo[3.2.1]octane (175.9 mg, 0.828 mmol) in DCM (5 mL) and DMF (1.25 mL). The reaction mixture was left to stir at 25° C. for 56 hrs. To the reaction was added water and DCM. The phases were separated. The aqueous phase was extracted with DCM (3×25 ml). The combined organics were dried using a phase separating filter and concentrated to afford tert-butyl 8-(4-bromo-7-chloro-2-methyl-pyrazolo[4,3-f]quinazolin-9-yl)-3,8-diazabicyclo[3.2.1]octane-3-carboxylate (382 mg, 0.752 mmol, 100% yield) as a yellow solid. Material was used without further purification.

UPLC-MS (ES⁺, Method 5): 2.24 min, m/z 509.0 [M+H]⁺.

Step B, 4-bromo-7-chloro-9-(3,8-diazabicyclo[3.2.1]octan-8-yl)-2-methyl-pyrazolo[4,3-f]quinazoline (1-87). Hydrogen Chloride in 1,4-dioxane (2.46 mL, 9.85 mmol) was added to a stirring solution of tert-butyl 8-(4-bromo-7-chloro-2-methyl-pyrazolo[4,3-f]quinazolin-9-yl)-3,8-diazabicyclo[3.2.1]octane-3-carboxylate (382 mg, 0.75 mmol) in DCM (4.92 mL). The reaction mixture was left to stir at 25° C. for 56 hr. To the reaction was added water and DCM. The phases were separated. The aqueous phase was extracted with DCM (3×25 ml). The combined organics were dried using a phase separating filter and concentrated to afford 4-bromo-7-chloro-9-(3,8-diazabicyclo[3.2.1]octan-8-yl)-2-methyl-pyrazolo[4,3-f]quinazoline (306 mg 0.751 mmol, 100% yield) as a yellow solid. Material was used without further purification.

UPLC-MS (ES⁺, Method 5): 1.44 min, m/z 409.0 [M+H]⁺.

Step C, 4-bromo-7-chloro-9-[3-(3-methoxypropyl)-3,8-diazabicyclo[3.2.1]octan-8-yl]-2-methyl-pyrazolo[4,3-f]quinazoline (1-88). 1-Bromo-3-methoxypropane (0.08 mL, 0.67 mmol) was added to a stirring solution of 4-bromo-7-chloro-9-(3,8-diazabicyclo[3.2.1]octan-8-yl)-2-methyl-pyrazolo[4,3-f]quinazoline (250. mg, 0.61 mmol) and Potassium carbonate (254.23 mg, 1.84 mmol) in DMF (3.07 mL). The reaction was stirred at 70° C. for 2 hrs. Solvent was evaporated to afford 4-bromo-7-chloro-9-[3-(3-methoxypropyl)-3,8-diazabicyclo[3.2.1]octan-8-yl]-2-methyl-pyrazolo[4,3-f]quinazoline (0.28 g, 0.584 mmol, 95% yield) (1-88) as a yellow solid.

UPLC-MS (ES⁺, Method 5): 1.53 min, m/z 481.0 [M+H]⁺.

The intermediates in table 8 below were prepared by analogy with 1-88 (intermediate scheme 37) replacing building blocks 3-Boc-3,8-Diazabicyclo[3.2.1]octane in step A and 1-Bromo-3-methoxypropane in step C as required.

TABLE 8

| | Intermediate | | UPLC-MS |
|---|---|---|---|
| Intermediate Building | Building block | Intermediate | (ES⁺): min, |
| block (Step A) | (Step C) | Structure/Name | m/z |
| 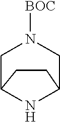 [SMILES: CC(C)(C)OC(=O)N1CC2CCC(C1)N2] 3-Boc-3,8- Diazabicyclo [3.2.1]octane | 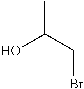 1-Bromo-2- propanol | Intermediate I-90 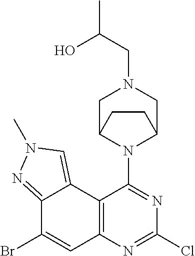 [SMILES: CC(O)CN1CC2CCC(C1)N2c1nc(Cl)nc2cc(Br)c3nn(C)cc3c12] | 1.46 min, 467.0 [M + H]⁺ (Method 5) |
| | | I-90 | |
| | | 1-[8-(4-bromo-7-chloro- | |
| | | 2-methyl-pyrazolo[4,3- | |
| | | f]quinazolin-9-yl)-3,8- | |
| | | diazabicyclo[3.2.1]octan- | |
| | | 3-yl]propan-2-ol | |
| 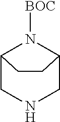 [SMILES: CC(C)(C)OC(=O)N1C2CCC1CNC2] 8-boc-3,8- diazabicyclo [3.2.1]octane | 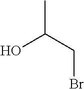 1-Bromo-2- propanol | Intermediate I-163 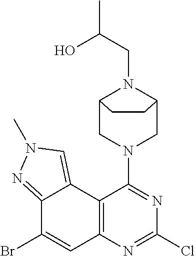 [SMILES: CC(O)CN1C2CCC1CN(c1nc(Cl)nc3cc(Br)c4nn(C)cc4c13)C2] | 1.44 min, 467.0 [M + H]⁺ (Method 5) |
| | | I-163 | |
| | | 1-[3-(4-bromo-7-chloro- | |
| | | 2-methyl-pyrazolo[4,3- | |
| | | f]quinazolin-9-yl)-3,8- | |
| | | diazabicyclo[3.2.1]octan- | |
| | | 8-yl]propan-2-ol | |

Intermediate I-91, 4-bromo-7-chloro-9-[3-[2-(methoxymethoxy) propyl]-3,8-diazabicyclo[3.2.1]octan-8-yl]-2-methyl-pyrazolo[4,3-f]quinazoline.

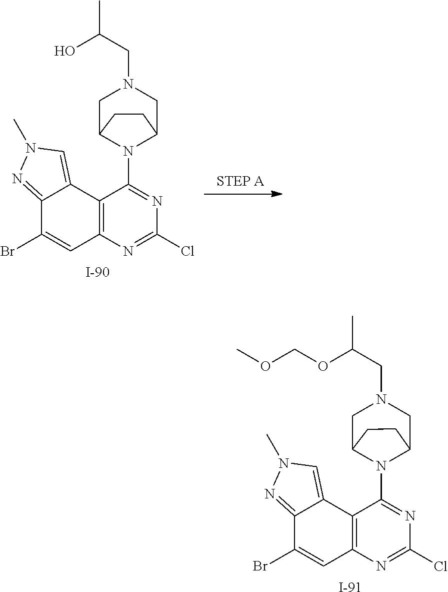

### Intermediate Scheme 38

Step A, 4-bromo-7-chloro-9-[3-[2-(methoxymethoxy) propyl]-3,8-diazabicyclo[3.2.1]octan-8-yl]-2-methyl-pyrazolo[4,3-f]quinazoline (I-91). To a solution of 1-[8-(4-bromo-7-chloro-2-methyl-pyrazolo[4,3-f]quinazolin-9-yl)-3,8-diazabicyclo[3.2.1]octan-3-yl]propan-2-ol (200 mg, 0.43 mmol) in DCM (4.30 mL) was added N, N-Diisopropylethylamine (0.22 mL, 1.29 mmol), the reaction was cooled to 0° C. and stirred for 5 min before the addition of Chloromethyl methyl ether (0.04 mL, 0.56 mmol). The reaction was stirred at 25° C. for 2 hrs. The mixture was extracted with DCM and water (×3) and the combined organic layer was then washed with water (×3) and brine (×1). Combined organics were dried with sodium sulfate, filtered and concentrated in vacuo to give 4-bromo-7-chloro-9-[3-[2-(methoxymethoxy) propyl]-3,8-diazabicyclo[3.2.1]octan-8-yl]-2-methyl-pyrazolo[4,3-f]quinazoline (218 mg, 0.42 mmol, 100% yield) as a yellow solid, which was used in the next step directly without purification.

UPLC-MS (ES⁺, Method 5): 1.57 min, m/z 511.0 [M+H]⁺.

The intermediates in table 9 below were prepared by analogy with I-91 (intermediate scheme 38) replacing building blocks for step A as described in the table entry.

TABLE 9

| | Intermediate | |
|---|---|---|
| Intermediate Building block | Structure/Name | UPLC-MS (ES⁺): min, m/z |
| 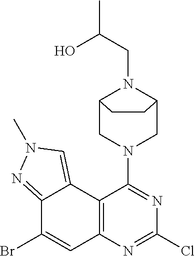 | 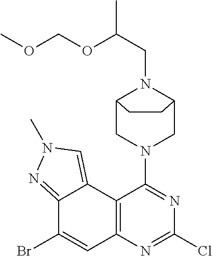 [SMILES: COCOC(C)CN1C2CCC1CN(c1nc(Cl)nc3cc(Br)c4nn(C)cc4c13)C2] | 1.59 min, 509.0/511.0 [M + H]⁺ (Method 5) |
| Intermediate I-163 | Intermediate I-164 | |
| I-163 | I-164 | |
| 1-[3-(4-bromo-7-chloro-2- | 4-bromo-7-chloro-9-[8-[2- | |
| methyl-pyrazolo[4,3- | (methoxymethoxy)propyl]- | |
| f]quinazolin-9-yl)-3,8- | 3,8- | |
| diazabicyclo[3.2.1]octan-8- | diazabicyclo[3.2.1]octan-3- | |
| yl]propan-2-ol | yl]-2-methyl-pyrazolo[4,3- | |
| | f]quinazoline | |

Intermediate 1-3, 4-[4-bromo-2-methyl-7-[[(2R,8S)-2-fluoro-1,2,3,5,6,7-hexahydropyrrolizin-8-yl]methoxy]pyrazolo[4,3-f]quinazolin-9-yl]-1,4-oxazepane.

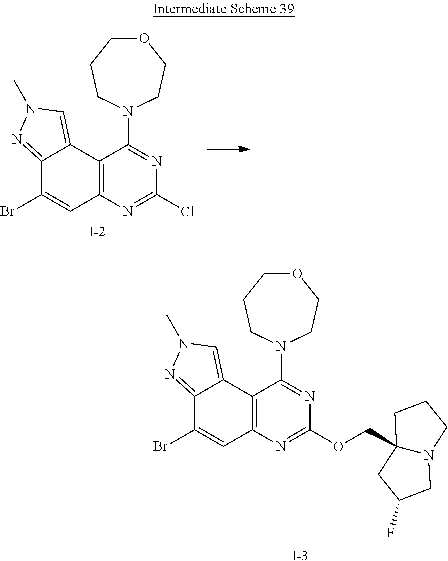

4-[4-bromo-2-methyl-7-[[(2R,8S)-2-fluoro-1,2,3,5,6,7-hexahydropyrrolizin-8-yl]methoxy]pyrazolo[4,3-f]quinazolin-9-yl]-1,4-oxazepane (1-3). Sodium hydride, (60% dispersed in mineral oil) (8.32 mg, 0.21 mmol) was added to a solution of ((2R,7aS)-2-fluorohexahydro-1H-pyrrolizin-7a-yl) methanol (28.7 mg, 0.18 mmol) in anhydrous THF (0.5 mL), and stirred at room temperature for 45 minutes. 4-(4-Bromo-7-chloro-2-methyl-pyrazolo[4,3-f]quinazolin-9-yl)-1,4-oxazepane (1-2) (55. mg, 0.14 mmol) in a solution of THF (0.4622 mL) and DMF (0.5 mL) was then added, and heated to 65° C. overnight. The reaction was quenched with 0.1 mL water and concentrated in vacuo. The residue was loaded onto Isolute HM-N with DCM and purified by flash column chromatography (5 g KP-amino functionalised silica, 5-80% EtOAc in petroleum ether). The desired fractions were concentrated to afford 4-[4-bromo-2-methyl-7-[[(2R,8S)-2-fluoro-1,2,3,5,6,7-hexahydropyrrolizin-8-yl]methoxy]pyrazolo[4,3-f]quinazolin-9-yl]-1,4-oxazepane (1-3) (40 mg, 0.077 mmol, 56% yield) as a colourless gum.

UPLC-MS (ES⁺, Method 2): 1.28 min, m/z 519.2, 521.2 [M+H]⁺.

The intermediates in table 10 below were prepared by analogy with I-3 (intermediate scheme 39) replacing intermediate 1-2 and building block ((2R,7aS)-2-fluorohexahydro-1H-pyrrolizin-7a-yl) methanol as described in the table entry.

TABLE 10

| Intermediate Building | | UPLC-MS (ES⁺): |
|---|---|---|
| block | Intermediate Structure/Name | min, m/z |
| 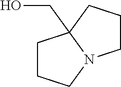 | 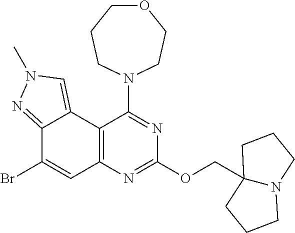 [SMILES: Cn1cc2c(n1)c(Br)cc1nc(OCC34CCCN3CCC4)nc(N3CCCOCC3)c12] | 0.66 min, 501.2/503.2 [M + H]⁺ (Method 4) |
| Intermediate I-2 and | Intermediate I-4 | |
| (tetrahydro-1H- | I-4 | |
| pyrrolizin-7a(5H)- | 4-[4-bromo-7-(1,2,3,5,6,7- | |
| yl)methanol | hexahydropyrrolizin-8- | |
| | ylmethoxy)-2-methyl- | |
| | pyrazolo[4,3-f]quinazolin-9-yl]- | |
| | 1,4-oxazepane | |
| 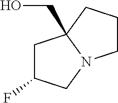 [SMILES: OC[C@@]12CCCN1C[C@H](F)C2] | 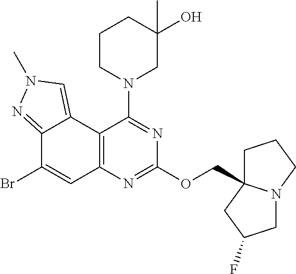 [SMILES: Cn1cc2c(n1)c(Br)cc1nc(OC[C@@]34CCCN3C[C@H](F)C4)nc(N3CCCC(C)(O)C3)c12] | 0.77 min, 533.1/535.1 [M + H]⁺ (Method 4) |
| Intermediate I-5 and | Intermediate I-6 | |
| ((2R,7aS)-2- | I-6 | |
| fluorohexahydro-1H- | 1-[4-bromo-2-methyl-7-[[(2R,8S)- | |
| pyrrolizin-7a- | 2-fluoro-1,2,3,5,6,7- | |
| yl)methanol | hexahydropyrrolizin-8- | |
| | yl]methoxy]pyrazolo[4,3- | |
| | f]quinazolin-9-yl]-3-methyl- | |
| | piperidin-3-ol | |
| 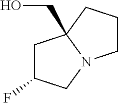 [SMILES: OC[C@@]12CCCN1C[C@H](F)C2] | 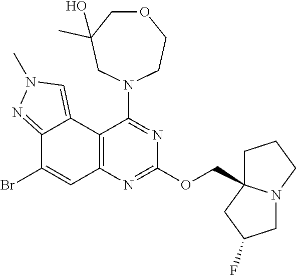 [SMILES: Cn1cc2c(n1)c(Br)cc1nc(OC[C@@]34CCCN3C[C@H](F)C4)nc(N3CCOCC(C)(O)C3)c12] | 0.89 min, 547.2/549.2 [M + H]⁺ (Method 4) |
| Intermediate I-7 and | Intermediate I-8 | |
| ((2R,7aS)-2- | I-8 | |
| fluorohexahydro-1H- | 4-[4-bromo-2-methyl-7-[[(2R,8S)- | |
| pyrrolizin-7a- | 2-fluoro-1,2,3,5,6,7- | |
| yl)methanol | hexahydropyrrolizin-8- | |
| | yl]methoxy]pyrazolo[4,3- | |
| | f]quinazolin-9-yl]-6-methyl-1,4- | |
| | oxazepan-6-ol | |
| 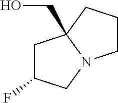 [SMILES: OC[C@@]12CCCN1C[C@H](F)C2] | 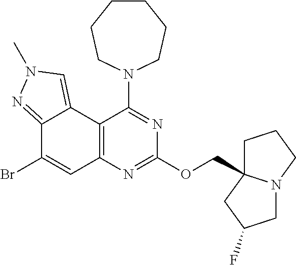 [SMILES: Cn1cc2c(n1)c(Br)cc1nc(OC[C@@]34CCCN3C[C@H](F)C4)nc(N3CCCCCC3)c12] | 1.09 min, 514.5/516.5 [M + H]⁺ (Method 4) |
| Intermediate I-9 and | Intermediate I-10 | |
| ((2R,7aS)-2- | I-10 | |
| fluorohexahydro-1H- | 1-[4-bromo-2-methyl-7-[[(2R,8S)- | |
| pyrrolizin-7a- | 2-fluoro-1,2,3,5,6,7- | |
| yl)methanol | hexahydropyrrolizin-8- | |
| | yl]methoxy]pyrazolo[4,3- | |
| | f]quinazolin-9-yl]azepane | |
| 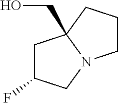 [SMILES: OC[C@@]12CCCN1C[C@H](F)C2] | 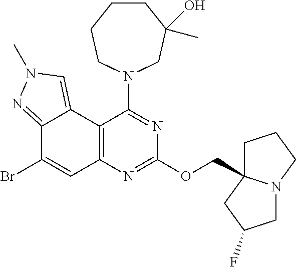 [SMILES: Cn1cc2c(n1)c(Br)cc1nc(OC[C@@]34CCCN3C[C@H](F)C4)nc(N3CCCCC(C)(O)C3)c12] | 0.83 min, 547.1/549.1 [M + H]⁺ (Method 4) |
| Intermediate I-11 and | Intermediate I-12 | |
| ((2R,7aS)-2- | I-12 | |
| fluorohexahydro-1H- | 1-[4-bromo-2-methyl-7-[[(2R,8S)- | |
| pyrrolizin-7a- | 2-fluoro-1,2,3,5,6,7- | |
| yl)methanol | hexahydropyrrolizin-8- | |
| | yl]methoxy]pyrazolo[4,3- | |
| | f]quinazolin-9-yl]-3-methyl- | |
| | azepan-3-ol | |
| 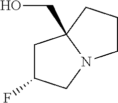 [SMILES: OC[C@@]12CCCN1C[C@H](F)C2] | 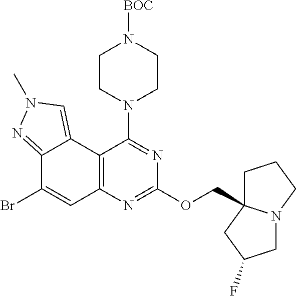 [SMILES: Cn1cc2c(n1)c(Br)cc1nc(OC[C@@]34CCCN3C[C@H](F)C4)nc(N3CCN(C(=O)OC(C)(C)C)CC3)c12] | 1.28 min, 604.2/606.2 [M + H]⁺ (Method 4) |
| Intermediate I-13 and | Intermediate I-14 | |
| ((2R,7aS)-2- | I-14 | |
| fluorohexahydro-1H- | tert-butyl 4-[4-bromo-2-methyl-7- | |
| pyrrolizin-7a- | [[(2R,8S)-2-fluoro-1,2,3,5,6,7- | |
| yl)methanol | hexahydropyrrolizin-8- | |
| | yl]methoxy]pyrazolo[4,3- | |
| | f]quinazolin-9-yl]piperazine-1- | |
| | carboxylate | |
| 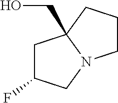 [SMILES: OC[C@@]12CCCN1C[C@H](F)C2] | 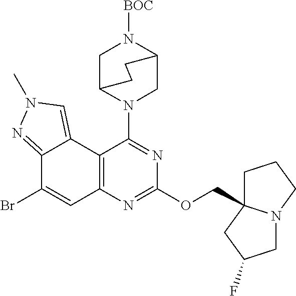 [SMILES: Cn1cc2c(n1)c(Br)cc1nc(OC[C@@]34CCCN3C[C@H](F)C4)nc(N3CC4CCC3CN4C(=O)OC(C)(C)C)c12] | 1.28 min, 630.2/632.2 [M + H]⁺ (Method 4) |
| Intermediate I-15 and | Intermediate I-16 | |
| ((2R,7aS)-2- | I-16 | |
| fluorohexahydro-1H- | tert-butyl 5-[4-bromo-2-methyl-7- | |
| pyrrolizin-7a- | [[(2R,8S)-2-fluoro-1,2,3,5,6,7- | |
| yl)methanol | hexahydropyrrolizin-8- | |
| | yl]methoxy]pyrazolo[4,3- | |
| | f]quinazolin-9-yl]-2,5- | |
| | diazabicyclo[2.2.2]octane-2- | |
| | carboxylate | |
| 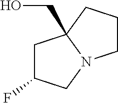 [SMILES: OC[C@@]12CCCN1C[C@H](F)C2] | 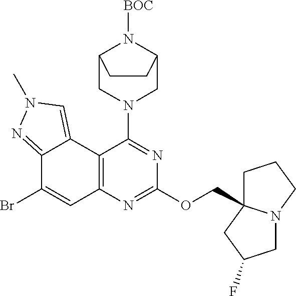 [SMILES: Cn1cc2c(n1)c(Br)cc1nc(OC[C@@]34CCCN3C[C@H](F)C4)nc(N3CC4CCC(C3)N4C(=O)OC(C)(C)C)c12] | 1.34 min, 630.2/632.2 [M + H]⁺ (Method 4) |
| Intermediate I-17 and | Intermediate I-18 | |
| ((2R,7aS)-2- | I-18 | |
| fluorohexahydro-1H- | tert-butyl 3-[4-bromo-2-methyl-7- | |
| pyrrolizin-7a- | [[(2R,8S)-2-fluoro-1,2,3,5,6,7- | |
| yl)methanol | hexahydropyrrolizin-8- | |
| | yl]methoxy]pyrazolo[4,3- | |
| | f]quinazolin-9-yl]-3,8- | |
| | diazabicyclo[3.2.1]octane-8- | |
| | carboxylate | |
| 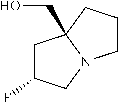 [SMILES: OC[C@@]12CCCN1C[C@H](F)C2] | 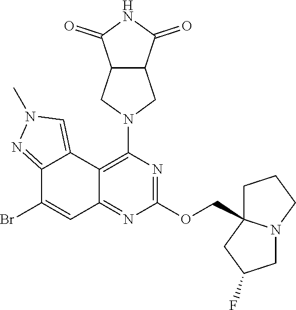 [SMILES: Cn1cc2c(n1)c(Br)cc1nc(OC[C@@]34CCCN3C[C@H](F)C4)nc(N3CC4C(=O)NC(=O)C4C3)c12] | 0.82 min, 558.0/560.0 [M + H]⁺ (Method 4) |
| Intermediate I-19 and | Intermediate I-20 | |
| ((2R,7aS)- | I-20 | |
| 2-fluorohexahydro-1H- | 5-[4-bromo-2-methyl-7-[[2R,8S)- | |
| pyrrolizin-7a- | 2-fluoro-1,2,3,5,6,7- | |
| yl)methanol | hexahydropyrrolizin-8- | |
| | yl]methoxy]pyrazolo[4,3- | |
| | f]quinazolin-9-yl]-3a,4,6,6a- | |
| | tetrahydropyrrolo[3,4-c]pyrrole- | |
| | 1,3-dione | |
| 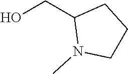 | 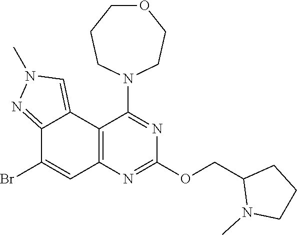 [SMILES: CN1CCCC1COc1nc(N2CCCOCC2)c2c(cc(Br)c3nn(C)cc32)n1] | 0.78 min, 475.1 [M + H]⁺ (Method 4) |
| Intermediate I-2 and | Intermediate I-21 | |
| (1-methylpyrrolidin-2- | I-21 | |
| yl)methanol | 4-[4-bromo-2-methyl-7-[(1- | |
| | methylpyrrolidin-2- | |
| | yl)methoxy]pyrazolo[4,3- | |
| | f]quinazolin-9-yl]-1,4-oxazepane | |
| 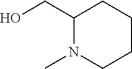 | 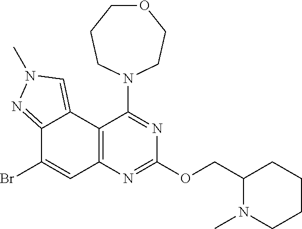 [SMILES: CN1CCCCC1COc1nc(N2CCCOCC2)c2c(cc(Br)c3nn(C)cc32)n1] | 3.13 min, 491.3 [M + H]⁺ (Method 4) |
| Intermediate I-2 and | Intermediate I-22 | |
| 1-Methyl-2- | I-22 | |
| piperidinemethanol | 4-[4-bromo-2-methyl-7-[(1- | |
| | methyl-2- | |
| | piperidyl)methoxy]pyrazolo[4,3- | |
| | f]quinazolin-9-yl]-1,4-oxazepane | |
| 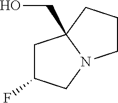 [SMILES: OC[C@@]12CCCN1C[C@H](F)C2] | 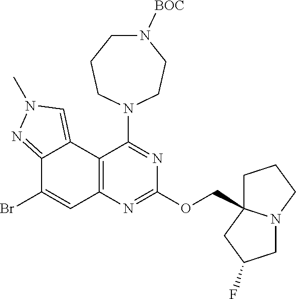 [SMILES: Cn1cc2c(n1)c(Br)cc1nc(OC[C@@]34CCCN3C[C@H](F)C4)nc(N3CCCN(C(=O)OC(C)(C)C)CC3)c12] | 2.88 min, 618.4 [M + H]⁺ (Method 4) |
| Intermediate I-23 and | Intermediate I-24 | |
| ((2R,7aS)- | I-24 | |
| 2-fluorohexahydro-1H- | tert-butyl 4-[4-bromo-2-methyl-7- | |
| pyrrolizin-7a- | [[(2R,8S)-2-fluoro-1,2,3,5,6,7- | |
| yl)methanol | hexahydropyrrolizin-8- | |
| | yl]methoxy]pyrazolo[4,3- | |
| | f]quinazolin-9-yl]-1,4-diazepane- | |
| | 1-carboxylate | |
| 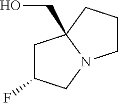 [SMILES: OC[C@@]12CCCN1C[C@H](F)C2] | 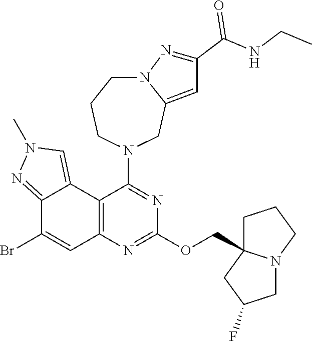 [SMILES: CCNC(=O)c1cc2n(n1)CCCN(c1nc(OC[C@@]34CCCN3C[C@H](F)C4)nc3cc(Br)c4nn(C)cc4c13)C2] | 0.97 min, 626.1 [M + H]⁺ (Method 4) |
| Intermediate I-25 and | Intermediate I-26 | |
| ((2R,7aS)- | I-26 | |
| 2-fluorohexahydro-1H- | 5-[4-bromo-2-methyl-7-[[(2R,8S)- | |
| pyrrolizin-7a- | 2-fluoro-1,2,3,5,6,7- | |
| yl)methanol | hexahydropyrrolizin-8- | |
| | yl]methoxy]pyrazolo[4,3- | |
| | f]quinazolin-9-yl]-N-ethyl-4,6,7,8- | |
| | tetrahydropyrazolo[1,5- | |
| | a][1,4]diazepine-2-carboxamide | |
| 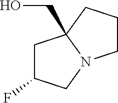 [SMILES: OC[C@@]12CCCN1C[C@H](F)C2] | 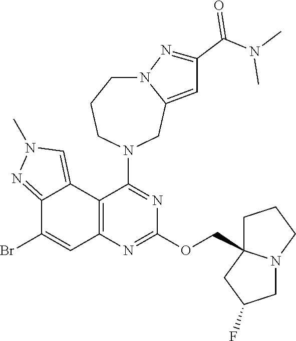 [SMILES: CN(C)C(=O)c1cc2n(n1)CCCN(c1nc(OC[C@@]34CCCN3C[C@H](F)C4)nc3cc(Br)c4nn(C)cc4c13)C2] | 0.93 min, 626.2 [M + H]⁺ (Method 4) |
| Intermediate I-27 and | Intermediate I-28 | |
| ((2R,7aS)- | I-28 | |
| 2-fluorohexahydro-1H- | 5-[4-bromo-2-methyl-7-[[(2R,8S)- | |
| pyrrolizin-7a- | 2-fluoro-1,2,3,5,6,7- | |
| yl)methanol | hexahydropyrrolizin-8- | |
| | yl]methoxy]pyrazolo[4,3- | |
| | f]quinazolin-9-yl]-N,N-dimethyl- | |
| | 4,6,7,8-tetrahydropyrazolo[1,5- | |
| | a][1,4]diazepine-2-carboxamide | |
| 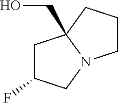 [SMILES: OC[C@@]12CCCN1C[C@H](F)C2] | 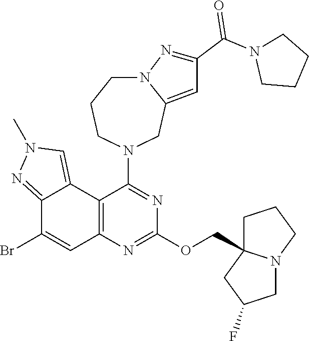 [SMILES: Cn1cc2c(n1)c(Br)cc1nc(OC[C@@]34CCCN3C[C@H](F)C4)nc(N3CCCn4nc(C(=O)N5CCCC5)cc4C3)c12] | 0.92 min, 652.2 [M + H]⁺ (Method 4) |
| Intermediate I-29 and | Intermediate I-30 | |
| ((2R,7aS)- | I-30 | |
| 2-fluorohexahydro-1H- | [5-[4-bromo-2-methyl-7- | |
| pyrrolizin-7a- | [[(2R,8S)-2-fluoro-1,2,3,5,6,7- | |
| yl)methanol | hexahydropyrrolizin-8- | |
| | yl]methoxy]pyrazolo[4,3- | |
| | f]quinazolin-9-yl]-4,6,7,8- | |
| | tetrahydropyrazolo[1,5- | |
| | a][1,4]diazepin-2-yl]-pyrrolidin-1- | |
| | yl-methanone | |
| 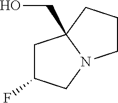 [SMILES: OC[C@@]12CCCN1C[C@H](F)C2] | 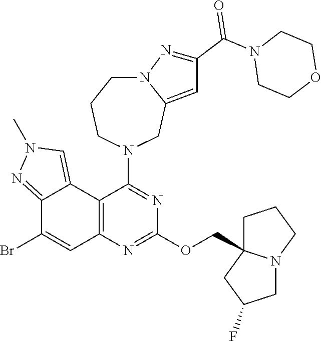 [SMILES: Cn1cc2c(n1)c(Br)cc1nc(OC[C@@]34CCCN3C[C@H](F)C4)nc(N3CCCn4nc(C(=O)N5CCOCC5)cc4C3)c12] | 0.77 min, 668.2/670.2 [M + H]⁺ (Method 4) |
| Intermediate I-31 and | Intermediate I-32 | |
| ((2R,7aS)- | I-32 | |
| 2-fluorohexahydro-1H- | [5-[4-bromo-2-methyl-7- | |
| pyrrolizin-7a- | [[(2R,8S)-2-fluoro-1,2,3,5,6,7- | |
| yl)methanol | hexahydropyrrolizin-8- | |
| | yl]methoxy]pyrazolo[4,3- | |
| | f]quinazolin-9-yl]-4,6,7,8- | |
| | tetrahydropyrazolo[1,5- | |
| | a][1,4]diazepin-2-yl]-morpholino- | |
| | methanone | |
| 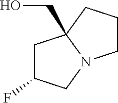 [SMILES: OC[C@@]12CCCN1C[C@H](F)C2] | 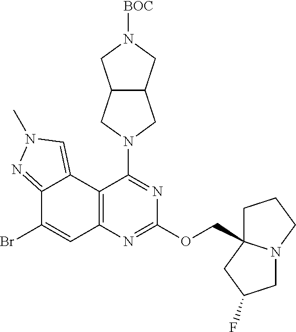 [SMILES: Cn1cc2c(n1)c(Br)cc1nc(OC[C@@]34CCCN3C[C@H](F)C4)nc(N3CC4CN(C(=O)OC(C)(C)C)CC4C3)c12] | 1.11 min, 630.0/632.0 [M + H]⁺ (Method 4) |
| Intermediate I-33 and | Intermediate I-34 | |
| ((2R,7aS)- | I-34 | |
| 2-fluorohexahydro-1H- | tert-butyl 2-[4-bromo-2-methyl-7- | |
| pyrrolizin-7a- | [[(2R,8S)-2-fluoro-1,2,3,5,6,7- | |
| yl)methanol | hexahydropyrrolizin-8- | |
| | yl]methoxy]pyrazolo[4,3- | |
| | f]quinazolin-9-yl]-1,3,3a,4,6,6a- | |
| | hexahydropyrrolo[3,4-c]pyrrole-5- | |
| | carboxylate | |
| 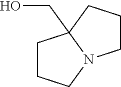 | 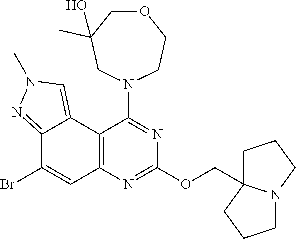 [SMILES: Cn1cc2c(n1)c(Br)cc1nc(OCC34CCCN3CCC4)nc(N3CCOCC(C)(O)C3)c12] | 0.79 min, 531.1/533.2 [M + H]⁺ (Method 4) |
| Intermediate I-7 and | Intermediate I-35 | |
| (tetrahydro-1H- | I-35 | |
| pyrrolizin-7a(5H)- | 4-[4-bromo-7-(1,2,3,5,6,7- | |
| yl)methanol | hexahydropyrrolizin-8- | |
| | ylmethoxy)-2-methyl- | |
| | pyrazolo[4,3-f]quinazolin-9-yl]-6- | |
| | methyl-1,4-oxazepan-6-ol | |
| 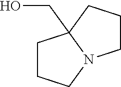 | 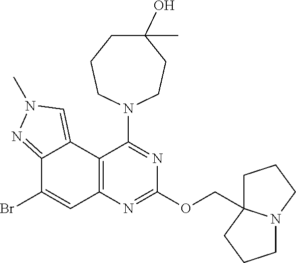 [SMILES: Cn1cc2c(n1)c(Br)cc1nc(OCC34CCCN3CCC4)nc(N3CCCC(C)(O)CC3)c12] | 1.55 min, 529.3 [M + H]⁺ (Method 4) |
| Intermediate I-36 and | Intermediate I-37 | |
| (tetrahydro-1H- | I-37 | |
| pyrrolizin-7a(5H)- | 1-[4-bromo-7-(1,2,3,5,6,7- | |
| yl)methanol | hexahydropyrrolizin-8- | |
| | ylmethoxy)-2-methyl- | |
| | pyrazolo[4,3-f]quinazolin-9-yl]-4- | |
| | methyl-azepan-4-ol | |
| 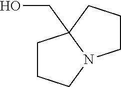 | 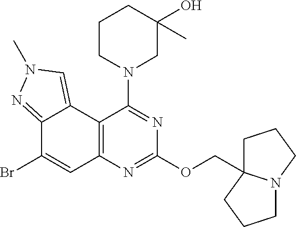 | 0.77 min, 533.1/535.1 [M + H]⁺ (Method 4) |
| Intermediate I-5 and | Intermediate I-38 | |
| (tetrahydro-1H- | I-38 | |
| pyrrolizin-7a(5H)- | 1-[4-bromo-7-(1,2,3,5,6,7- | |
| yl)methanol | hexahydropyrrolizin-8- | |
| | ylmethoxy)-2-methyl- | |
| | pyrazolo[4,3-f]quinazolin-9-yl]-3- | |
| | methyl-piperidin-3-ol | |
| 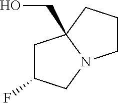 [SMILES: OC[C@@]12CCCN1C[C@H](F)C2] | 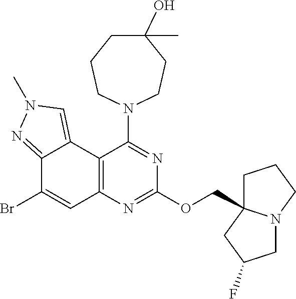 [SMILES: Cn1cc2c(n1)c(Br)cc1nc(OC[C@@]34CCCN3C[C@H](F)C4)nc(N3CCCC(C)(O)CC3)c12] | 1.40 min, 547.30 [M + H]⁺ (Method 4) |
| Intermediate I-36 and | Intermediate I-39 | |
| ((2R,7aS)- | I-39 | |
| 2-fluorohexahydro-1H- | 1-[4-bromo-2-methyl-7-[[(2R,8S)- | |
| pyrrolizin-7a- | 2-fluoro-1,2,3,5,6,7- | |
| yl)methanol | hexahydropyrrolizin-8- | |
| | yl]methoxy]pyrazolo[4,3- | |
| | f]quinazolin-9-yl]-4-methyl- | |
| | azepan-4-ol | |
| 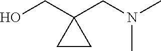 | 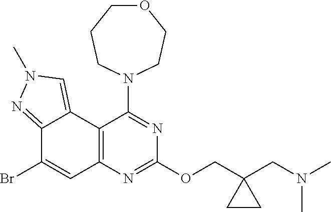 [SMILES: CN(C)CC1(COc2nc(N3CCCOCC3)c3c(cc(Br)c4nn(C)cc43)n2)CC1] | 0.77 min, 489.1 [M + H]⁺ (Method 4) |
| Intermediate I-2 and | Intermediate I-40 | |
| ((dimethylamino)methyl) | I-40 | |
| cyclopropyl)methanol | 1-[1-[[4-bromo-2-methyl-9-(1,4- | |
| | oxazepan-4-yl)pyrazolo[4,3- | |
| | f]quinazolin-7- | |
| | yl]oxymethyl]cyclopropyl]-N,N- | |
| | dimethyl-methanamine | |
| 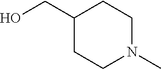 | 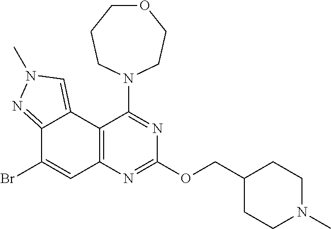 [SMILES: CN1CCC(COc2nc(N3CCCOCC3)c3c(cc(Br)c4nn(C)cc43)n2)CC1] | 0.43 min, 245.1 [M + H]⁺ (Method 4) |
| Intermediate I-2 and | Intermediate I-41 | |
| 1-methyl-4- | I-41 | |
| piperidinemethanol | 4-[4-bromo-2-methyl-7-[(1- | |
| | methyl-4- | |
| | piperidyl)methoxy]pyrazolo[4,3- | |
| | f]quinazolin-9-yl]-1,4-oxazepane | |
| 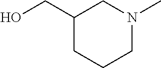 | 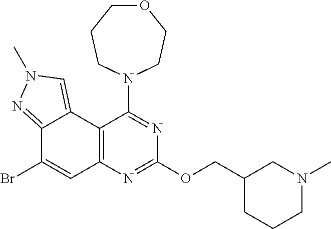 [SMILES: CN1CCCC(COc2nc(N3CCCOCC3)c3c(cc(Br)c4nn(C)cc43)n2)C1] | 1.13 min, 489.1 [M + H]⁺ (Method 4) |
| Intermediate I-2 and | Intermediate I-42 | |
| | I-42 | |
| | 4-[4-bromo-2-methyl-7-[(1- | |
| | methyl-3- | |
| | piperidyl)methoxy]pyrazolo[4,3- | |
| | f]quinazolin-9-yl]-1,4-oxazepane | |
| Chiral separation of I-8 | 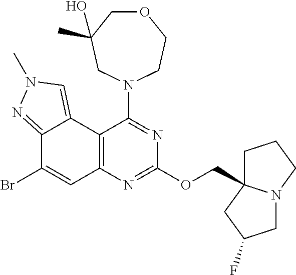 [SMILES: Cn1cc2c(n1)c(Br)cc1nc(OC[C@@]34CCCN3C[C@H](F)C4)nc(N3CCOC[C@@](C)(O)C3)c12] | 2.38 min, 551.4 [M + H]⁺ (Method 3) |
| | Intermediate I-45 | |
| | I-45 | |
| | rac-(6S)-4-[4-bromo-2-methyl-7- | |
| | [[rac-(2R,8S)-2-fluoro- | |
| | 1,2,3,5,6,7-hexahydropyrrolizin- | |
| | 8-yl]methoxy]pyrazolo[4,3- | |
| | f]quinazolin-9-yl]-6-methyl-1,4- | |
| | oxazepan-6-ol | |
| Chiral separation of I-8 | 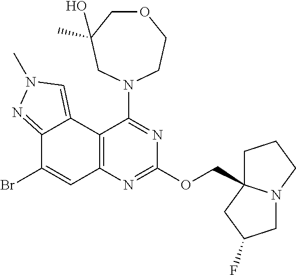 [SMILES: Cn1cc2c(n1)c(Br)cc1nc(OC[C@@]34CCCN3C[C@H](F)C4)nc(N3CCOC[C@](C)(O)C3)c12] | 2.38 min, m/z, 551.4 [M + H]⁺ (Method 3) |
| | Intermediate I-46 | |
| | I-46 | |
| | 6R)-4-[4-bromo-2-methyl-7-[rac- | |
| | (2R,8S)-2-fluoro-1,2,3,5,6,7- | |
| | hexahydropyrrolizin-8- | |
| | yl]methoxy]pyrazolo[4,3- | |
| | f]quinazolin-9-yl]-6-methyl-1,4- | |
| | oxazepan-6-ol | |
| 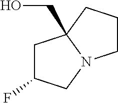 [SMILES: OC[C@@]12CCCN1C[C@H](F)C2] | 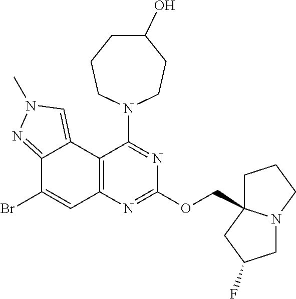 [SMILES: Cn1cc2c(n1)c(Br)cc1nc(OC[C@@]34CCCN3C[C@H](F)C4)nc(N3CCCC(O)CC3)c12] | 1.23 min, 533.0 [M + H]⁺ (Method 4) |
| Intermediate I-47 and | Intermediate I-48 | |
| ((2R,7aS)- | I-48 | |
| 2-fluorohexahydro-1H- | 1-[4-bromo-2-methyl-7-[[(2R,8S)- | |
| pyrrolizin-7a- | 2-fluoro-1,2,3,5,6,7- | |
| yl)methanol | hexahydropyrrolizin-8- | |
| | yl]methoxy]pyrazolo[4,3- | |
| | f]quinazolin-9-yl]azepan-4-ol | |
| 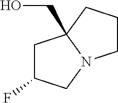 [SMILES: OC[C@@]12CCCN1C[C@H](F)C2] | 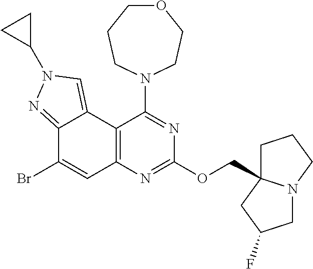 [SMILES: F[C@H]1CN2CCC[C@@]2(COc2nc(N3CCCOCC3)c3c(cc(Br)c4nn(C5CC5)cc43)n2)C1] | 1.09 min, 545.2 [M + H]⁺ (Method 4) |
| Intermediate I-50 and | Intermediate I-51 | |
| ((2R,7aS)- | I-51 | |
| 2-fluorohexahydro-1H- | 4-[4-bromo-2-cyclopropyl-7- | |
| pyrrolizin-7a- | [[(2R,8S)-2-fluoro-1,2,3,5,6,7- | |
| yl)methanol | hexahydropyrrolizin-8- | |
| | yl]methoxy]pyrazolo[4,3- | |
| | f]quinazolin-9-yl]-1,4-oxazepane | |
| 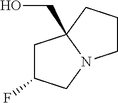 [SMILES: OC[C@@]12CCCN1C[C@H](F)C2] | 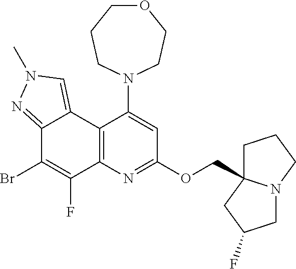 [SMILES: Cn1cc2c(n1)c(Br)c(F)c1nc(OC[C@@]34CCCN3C[C@H](F)C4)cc(N3CCCOCC3)c12] | 1.07 min, 536.1 [M + H]⁺ (Method 4) |
| Intermediate I-53 and | Intermediate I-54 | |
| ((2R,7aS)- | I-54 | |
| 2-fluorohexahydro-1H- | 4-[4-bromo-5-fluoro-2-methyl-7- | |
| pyrrolizin-7a- | [[(2R,8S)-2-fluoro-1,2,3,5,6,7- | |
| yl)methanol | hexahydropyrrolizin-8- | |
| | yl]methoxy]pyrazolo[4,3- | |
| | f]quinolin-9-yl]-1,4-oxazepane | |
| 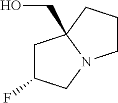 [SMILES: OC[C@@]12CCCN1C[C@H](F)C2] | 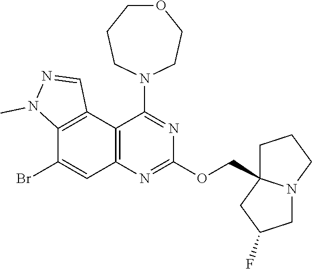 [SMILES: Cn1ncc2c3c(N4CCCOCC4)nc(OC[C@@]45CCCN4C[C@H](F)C5)nc3cc(Br)c21] | 0.91 min, 519.0/521.0 [M + H]⁺ (Method 4) |
| Intermediate I-56 and | Intermediate I-57 | |
| ((2R,7aS)- | I-57 | |
| 2-fluorohexahydro-1H- | 4-[4-bromo-3-methyl-7-[[(2R,8S)- | |
| pyrrolizin-7a- | 2-fluoro-1,2,3,5,6,7- | |
| yl)methanol | hexahydropyrrolizin-8- | |
| | yl]methoxy]pyrazolo[4,3- | |
| | f]quinazolin-9-yl]-1,4-oxazepane | |
| 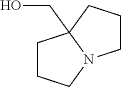 | 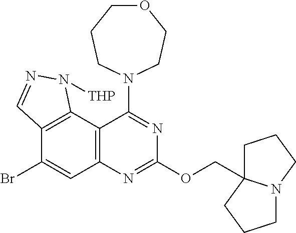 | 2.14 min, 571.1/573.1 [M + H]⁺ (Method 2) |
| Intermediate I-58 and | Intermediate I-59 | |
| (tetrahydro-1H- | I-59 | |
| pyrrolizin-7a(5H)- | 4-[4-bromo-7-(1,2,3,5,6,7- | |
| yl)methanol | hexahydropyrrolizin-8- | |
| | ylmethoxy)-1-tetrahydropyran-2- | |
| | yl-pyrazolo[3,4-f]quinazolin-9-yl]- | |
| | 1,4-oxazepane | |
| 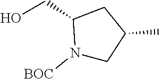 | 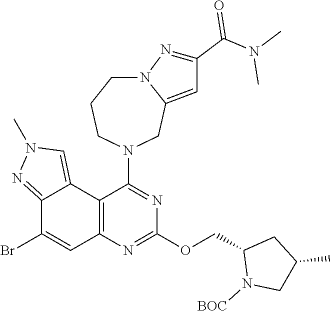 [SMILES: C[C@H]1C[C@@H](COc2nc(N3CCCn4nc(C(=O)N(C)C)cc4C3)c3c(cc(Br)c4nn(C)cc43)n2)N(C(=O)OC(C)(C)C)C1] | 1.38 min, 626.2/628.2 [M + H]⁺ (Method 5) |
| Intermediate I-27 and | Intermediate I-60 | |
| (2S,4S)-tert-butyl 2- | I-60 | |
| (heydroxymethyl)-4- | tert-butyl rac-(2S,4S)-2-[4- | |
| methylpyrrolidine-1- | bromo-9-[2-(dimethylcarbamoyl)- | |
| carboxylate | 4,6,7,8-tetrahydropyrazolo[1,5- | |
| | a][1,4]diazepin-5-yl]-2-methyl- | |
| | pyrazolo[4,3-f]quinazolin-7- | |
| | yl]oxymethyl]-4-methyl- | |
| | pyrrolidine-1-carboxylate | |
| 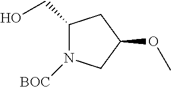 | 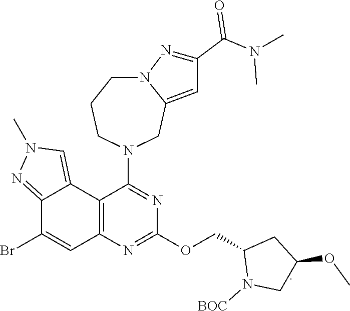 | 2.06 min, 852.6 [M + H]⁺ (Method 2) |
| Intermediate I-27 and | Intermediate I-61 | |
| tert-butyl (2S,4R)-2- | I-61 | |
| (hydroxymethyl)-4- | tert-butyl (2S,4R)-2-[9-[2- | |
| methoxy-pyrrolidine-1- | (dimethylcarbamoyl)-4,6,7,8- | |
| carboxylate | tetrahydropyrazolo[1,5- | |
| | a][1,4]diazepin-5-yl]-4-[8-ethyl-7- | |
| | fluoro-3-(methoxymethoxy)-1- | |
| | naphthyl]-2-methyl-pyrazolo[4,3- | |
| | f]quinazolin-7-yl]oxymethyl]-4- | |
| | methoxy-pyrrolidine-1- | |
| | carboxylate | |
| 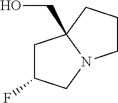 [SMILES: OC[C@@]12CCCN1C[C@H](F)C2] | 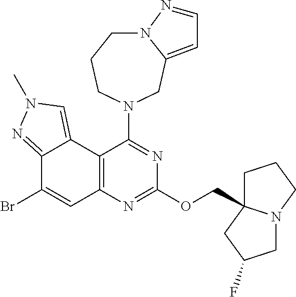 [SMILES: Cn1cc2c(n1)c(Br)cc1nc(OC[C@@]34CCCN3C[C@H](F)C4)nc(N3CCCn4nccc4C3)c12] | 1.18 min, 557.2 [M + H]⁺ (Method 2) |
| Intermediate I-64 and | Intermediate I-65 | |
| ((2R,7aS)- | I-65 | |
| 2-fluorohexahydro-1H- | 4-bromo-2-methyl-7-[[(2R,8S)-2- | |
| pyrrolizin-7a- | fluoro-1,2,3,5,6,7- | |
| yl)methanol | hexahydropyrrolizin-8- | |
| | yl]methoxy]-9-(4,6,7,8- | |
| | tetrahydropyrazolo[1,5- | |
| | a][1,4]diazepin-5-yl)pyrazolo[4,3- | |
| | f]quinazoline | |
| 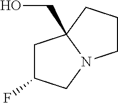 [SMILES: OC[C@@]12CCCN1C[C@H](F)C2] | 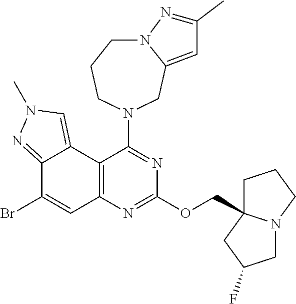 [SMILES: Cc1cc2n(n1)CCCN(c1nc(OC[C@@]34CCCN3C[C@H](F)C4)nc3cc(Br)c4nn(C)cc4c13)C2] | 1.36 min, 571.1 [M + H]⁺ (Method 5) |
| Intermediate I-66 and | Intermediate I-67 | |
| ((2R,7aS)- | I-67 | |
| 2-fluorohexahydro-1H- | 4-bromo-7-[[(2R,8S)-2-fluoro- | |
| pyrrolizin-7a- | 1,2,3,5,6,7-hexahydropyrrolizin- | |
| yl)methanol | 8-yl]methoxy]-2-methyl-9-(2- | |
| | methyl-4,6,7,8- | |
| | tetrahydropyrazolo[1,5- | |
| | a][1,4]diazepin-5-yl)pyrazolo[4,3- | |
| | f]quinazoline | |
| 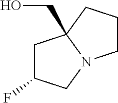 [SMILES: OC[C@@]12CCCN1C[C@H](F)C2] | 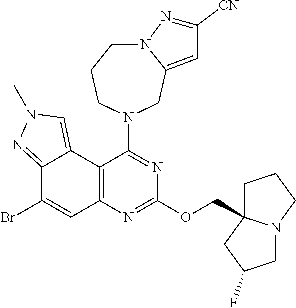 [SMILES: Cn1cc2c(n1)c(Br)cc1nc(OC[C@@]34CCCN3C[C@H](F)C4)nc(N3CCCn4nc(C#N)cc4C3)c12] | 1.45 min, 582.1 [M + H]⁺ (Method 5) |
| Intermediate I-68 and | Intermediate I-69 | |
| ((2R,7aS)- | I-69 | |
| 2-fluorohexahydro-1H- | 5-[4-bromo-2-methyl-7-[[(2R,8S)- | |
| pyrrolizin-7a- | 2-fluoro-1,2,3,5,6,7- | |
| yl)methanol | hexahydropyrrolizin-8- | |
| | yl]methoxy]pyrazolo[4,3- | |
| | f]quinazolin-9-yl]-4,6,7,8- | |
| | tetrahydropyrazolo[1,5- | |
| | a][1,4]diazepine-2-carbonitrile | |
| 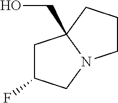 [SMILES: OC[C@@]12CCCN1C[C@H](F)C2] | 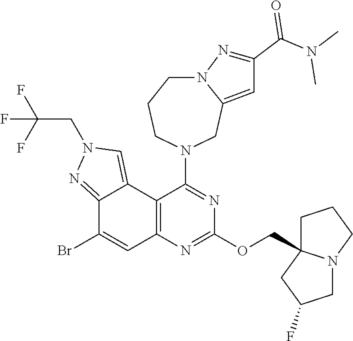 [SMILES: CN(C)C(=O)c1cc2n(n1)CCCN(c1nc(OC[C@@]34CCCN3C[C@H](F)C4)nc3cc(Br)c4nn(CC(F)(F)F)cc4c13)C2] | 1.44 min, 696.3 [M + H]⁺ (Method 2) |
| Intermediate I-73 and | Intermediate I-74 | |
| ((2R,7aS)- | I-74 | |
| 2-fluorohexahydro-1H- | 5-[4-bromo-7-[[(2R,8S)-2-fluoro- | |
| pyrrolizin-7a- | 1,2,3,5,6,7-hexahydropyrrolizin- | |
| yl)methanol | 8-yl]methoxy]-2-(2,2,2- | |
| | trifluoroethyl)pyrazolo[4,3- | |
| | f]quinazolin-9-yl]-N,N-dimethyl- | |
| | 4,6,7,8-tetrahydropyrazolo[1,5- | |
| | a][1,4]diazepine-2-carboxamide | |
| 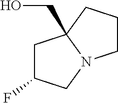 [SMILES: OC[C@@]12CCCN1C[C@H](F)C2] | 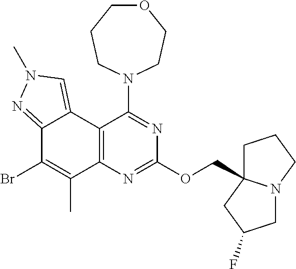 [SMILES: Cc1c(Br)c2nn(C)cc2c2c(N3CCCOCC3)nc(OC[C@@]34CCCN3C[C@H](F)C4)nc12] | 1.44 min, 535.1 [M + H]⁺ (Method 5) |
| Intermediate I-76 and | Intermediate I-77 | |
| ((2R,7aS)- | I-77 | |
| 2-fluorohexahydro-1H- | 4-[4-bromo-2,5-dimethyl-7- | |
| pyrrolizin-7a- | [[(2R,8S)-2-fluoro-1,2,3,5,6,7- | |
| yl)methanol | hexahydropyrrolizin-8- | |
| | yl]methoxy]pyrazolo[4,3- | |
| | f]quinazolin-9-yl]-1,4-oxazepane | |
| 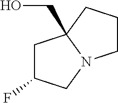 [SMILES: OC[C@@]12CCCN1C[C@H](F)C2] | 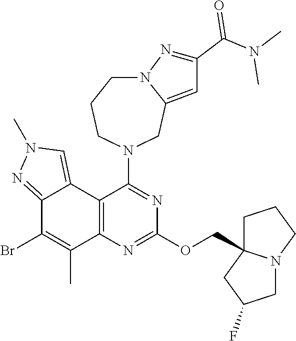 [SMILES: Cc1c(Br)c2nn(C)cc2c2c(N3CCCn4nc(C(=O)N(C)C)cc4C3)nc(OC[C@@]34CCCN3C[C@H](F)C4)nc12] | 1.31 min, 642.4 [M + H]⁺ (Method 2) |
| Intermediate I-78 and | Intermediate I-79 | |
| ((2R,7aS)- | I-79 | |
| 2-fluorohexahydro-1H- | 5-[4-bromo-2,5-dimethyl-7- | |
| pyrrolizin-7a- | [[(2R,8S)-2-fluoro-1,2,3,5,6,7- | |
| yl)methanol | hexahydropyrrolizin-8- | |
| | yl]methoxy]pyrazolo[4,3- | |
| | f]quinazolin-9-yl]-N,N-dimethyl- | |
| | 4,6,7,8-tetrahydropyrazolo[1,5- | |
| | a][1,4]diazepine-2-carboxamide | |
| 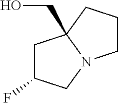 [SMILES: OC[C@@]12CCCN1C[C@H](F)C2] | 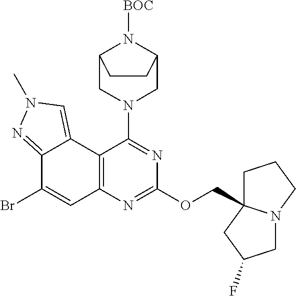 [SMILES: Cn1cc2c(n1)c(Br)cc1nc(OC[C@@]34CCCN3C[C@H](F)C4)nc(N3CC4CCC(C3)N4C(=O)OC(C)(C)C)c12] | 1.69 min, 632.2 [M + H]⁺ (Method 5) |
| Intermediate I-81 and | Intermediate I-82 | |
| ((2R,7aS)- | I-82 | |
| 2-fluorohexahydro-1H- | tert-butyl 3-[4-bromo-7-[[(2R,8S)- | |
| pyrrolizin-7a- | 2-fluoro-1,2,3,5,6,7- | |
| yl)methanol | hexahydropyrrolizin-8- | |
| | yl]methoxy]-2-methyl- | |
| | pyrazolo[4,3-f]quinazolin-9-yl]- | |
| | 3,8-diazabicyclo[3.2.1]octane-8- | |
| | carboxylate | |
| 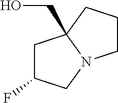 [SMILES: OC[C@@]12CCCN1C[C@H](F)C2] | 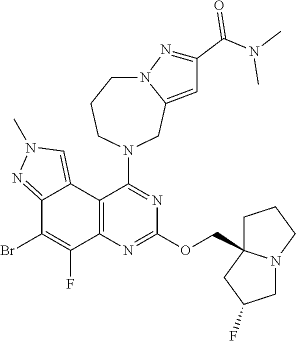 [SMILES: CN(C)C(=O)c1cc2n(n1)CCCN(c1nc(OC[C@@]34CCCN3C[C@H](F)C4)nc3c(F)c(Br)c4nn(C)cc4c13)C2] | 1.38 min, 646.1 [M + H]⁺ (Method 5) |
| Intermediate I-83 and | Intermediate I-84 | |
| ((2R,7aS)- | I-84 | |
| 2-fluorohexahydro-1H- | 5-[4-bromo-5-fluoro-2-methyl-7- | |
| pyrrolizin-7a- | [[(2R,8S)-2-fluoro-1,2,3,5,6,7- | |
| yl)methanol | hexahydropyrrolizin-8- | |
| | yl]methoxy]pyrazolo[4,3- | |
| | f]quinazolin-9-yl]-N,N-dimethyl- | |
| | 4,6,7,8-tetrahydropyrazolo[1,5- | |
| | a][1,4]diazepine-2-carboxamide | |
| 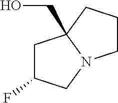 [SMILES: OC[C@@]12CCCN1C[C@H](F)C2] | 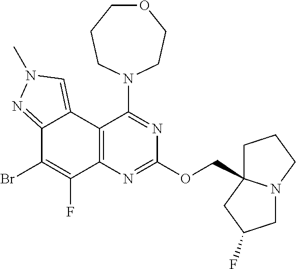 [SMILES: Cn1cc2c(n1)c(Br)c(F)c1nc(OC[C@@]34CCCN3C[C@H](F)C4)nc(N3CCCOCC3)c12] | 1.37 min, 539.0 [M + H]⁺ (Method 5) |
| Intermediate I-85 and | Intermediate I-86 | |
| ((2R,7aS)- | I-86 | |
| 2-fluorohexahydro-1H- | 4-[4-bromo-5-fluoro-2-methyl-7- | |
| pyrrolizin-7a- | [[(2R,8S)-2-fluoro-1,2,3,5,6,7- | |
| yl)methanol | hexahydropyrrolizin-8- | |
| | yl]methoxy]pyrazolo[4,3- | |
| | f]quinazolin-9-yl]-1,4-oxazepane | |
| 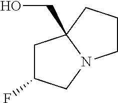 [SMILES: OC[C@@]12CCCN1C[C@H](F)C2] | 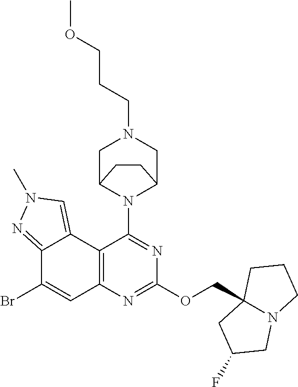 [SMILES: COCCCN1CC2CCC(C1)N2c1nc(OC[C@@]23CCCN2C[C@H](F)C3)nc2cc(Br)c3nn(C)cc3c12] | 1.28 min, 604.1 [M + H]⁺ (Method 5) |
| Intermediate I-88 and | Intermediate I-89 | |
| ((2R,7aS)- | I-89 | |
| 2-fluorohexahydro-1H- | 4-bromo-7-[[(2R,8S)-2-fluoro- | |
| pyrrolizin-7a- | 1,2,3,5,6,7-hexahydropyrrolizin- | |
| yl)methanol | 8-yl]methoxy]-9-[3-(3- | |
| | methoxypropyl)-3,8- | |
| | diazabicyclo[3.2.1]octan-8-yl]-2- | |
| | methyl-pyrazolo[4,3-f]quinazoline | |
| 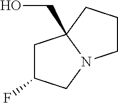 [SMILES: OC[C@@]12CCCN1C[C@H](F)C2] | 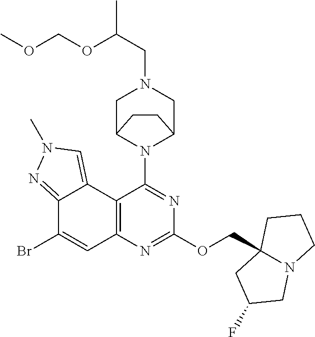 [SMILES: COCOC(C)CN1CC2CCC(C1)N2c1nc(OC[C@@]23CCCN2C[C@H](F)C3)nc2cc(Br)c3nn(C)cc3c12] | 1.21 min, 634.2 [M + H]⁺ (Method 5) |
| Intermediate I-91 and | Intermediate I-92 | |
| ((2R,7aS)- | I-92 | |
| 2-fluorohexahydro-1H- | 4-bromo-7-[[(2R,8S)-2-fluoro- | |
| pyrrolizin-7a- | 1,2,3,5,6,7-hexahydropyrrolizin- | |
| yl)methanol | 8-yl]methoxy]-9-[3-[2- | |
| | (methoxymethoxy)propyl]-3,8- | |
| | diazabicyclo[3.2.1]octan-8-yl]-2- | |
| | methyl-pyrazolo[4,3-f]quinazoline | |
| 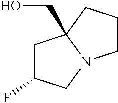 [SMILES: OC[C@@]12CCCN1C[C@H](F)C2] | 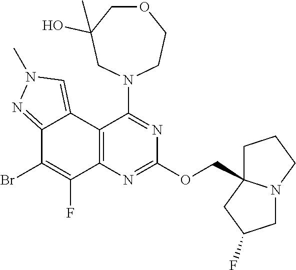 [SMILES: Cn1cc2c(n1)c(Br)c(F)c1nc(OC[C@@]34CCCN3C[C@H](F)C4)nc(N3CCOCC(C)(O)C3)c12] | 1.36 min, 569.0 [M + H]⁺ (Method 5) |
| Intermediate I-93 and | Intermediate I-94 | |
| ((2R,7aS)- | I-94 | |
| 2-fluorohexahydro-1H- | 4-[4-bromo-5-fluoro-2-methyl-7- | |
| pyrrolizin-7a- | [[(2R,8S)-2-fluoro-1,2,3,5,6,7- | |
| yl)methanol | hexahydropyrrolizin-8- | |
| | yl]methoxy]pyrazolo[4,3- | |
| | f]quinazolin-9-yl]-6-methyl-1,4- | |
| | oxazepan-6-ol | |
| 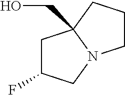 [SMILES: OC[C@@]12CCCN1C[C@H](F)C2] | 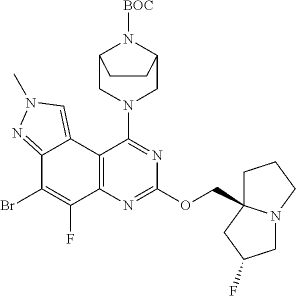 [SMILES: Cn1cc2c(n1)c(Br)c(F)c1nc(OC[C@@]34CCCN3C[C@H](F)C4)nc(N3CC4CCC(C3)N4C(=O)OC(C)(C)C)c12] | 1.70 min, 650.1 [M + H]⁺ (Method 5) |
| Intermediate I-103 and | Intermediate I-104 | |
| ((2R,7aS)- | I-104 | |
| 2-fluorohexahydro-1H- | tert-butyl 3-[4-bromo-5-fluoro-2- | |
| pyrrolizin-7a- | methyl-7-[[(2R,8S)-2-fluoro- | |
| yl)methanol | 1,2,3,5,6,7-hexahydropyrrolizin- | |
| | 8-yl]methoxy]pyrazolo[4,3- | |
| | f]quinazolin-9-yl]-3,8- | |
| | diazabicyclo[3.2.1]octane-8- | |
| | carboxylate | |
| 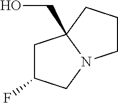 [SMILES: OC[C@@]12CCCN1C[C@H](F)C2] | 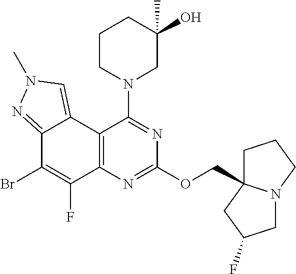 [SMILES: Cn1cc2c(n1)c(Br)c(F)c1nc(OC[C@@]34CCCN3C[C@H](F)C4)nc(N3CCC[C@@](C)(O)C3)c12] | 1.41 min, 553.0 [M + H]⁺ (Method 5) |
| Intermediate I-105 and | Intermediate I-106 | |
| ((2R,7aS)- | I-106 | |
| 2-fluorohexahydro-1H- | (3R)-1-[4-bromo-5-fluoro-2- | |
| pyrrolizin-7a- | methyl-7-[[(2R,8S)-2-fluoro- | |
| yl)methanol | 1,2,3,5,6,7-hexahydropyrrolizin- | |
| | 8-yl]methoxy]pyrazolo[4,3- | |
| | f]quinazolin-9-yl]-3-methyl- | |
| | piperidin-3-ol | |
| 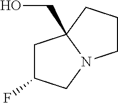 [SMILES: OC[C@@]12CCCN1C[C@H](F)C2] | 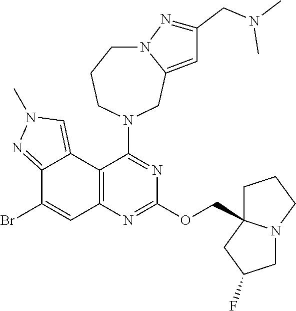 [SMILES: CN(C)Cc1cc2n(n1)CCCN(c1nc(OC[C@@]34CCCN3C[C@H](F)C4)nc3cc(Br)c4nn(C)cc4c13)C2] | 1.28 min, 614.1 [M + H]⁺ (Method 5) |
| Intermediate I-107 and | Intermediate I-108 | |
| ((2R,7aS)- | I-108 | |
| 2-fluorohexahydro-1H- | 1-[5-[4-bromo-2-methyl-7- | |
| pyrrolizin-7a- | [[(2R,8S)-2-fluoro-1,2,3,5,6,7- | |
| yl)methanol | hexahydropyrrolizin-8- | |
| | yl]methoxy]pyrazolo[4,3- | |
| | f]quinazolin-9-yl]-4,6,7,8- | |
| | tetrahydropyrazolo[1,5- | |
| | a][1,4]diazepin-2-yl]-N,N- | |
| | dimethyl-methanamine | |
| 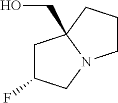 [SMILES: OC[C@@]12CCCN1C[C@H](F)C2] | 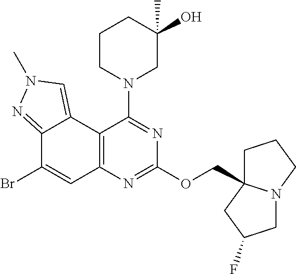 [SMILES: Cn1cc2c(n1)c(Br)cc1nc(OC[C@@]34CCCN3C[C@H](F)C4)nc(N3CCC[C@@](C)(O)C3)c12] | 1.37 min, 533.0 [M + H]⁺ (Method 5) |
| Intermediate I-113 and | Intermediate I-114 | |
| ((2R,7aS)- | I-114 | |
| 2-fluorohexahydro-1H- | (3R)-1-[4-bromo-7-[[(2R,8S)-2- | |
| pyrrolizin-7a- | fluoro-1,2,3,5,6,7- | |
| yl)methanol | hexahydropyrrolizin-8- | |
| | yl]methoxy]-2-methyl- | |
| | pyrazolo[4,3-f]quinazolin-9-yl]-3- | |
| | methyl-piperidin-3-ol | |
| 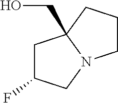 [SMILES: OC[C@@]12CCCN1C[C@H](F)C2] | 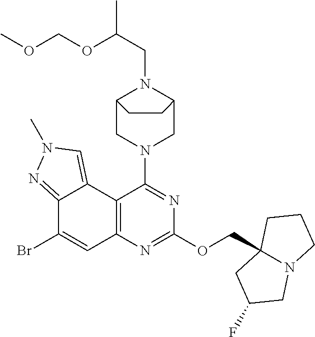 [SMILES: COCOC(C)CN1C2CCC1CN(c1nc(OC[C@@]34CCCN3C[C@H](F)C4)nc3cc(Br)c4nn(C)cc4c13)C2] | 1.27 min, 632.2/634.2 [M + H]⁺ (Method 5) |
| Intermediate I-164 and | Intermediate I-165 | |
| ((2R,7aS)- | I-165 | |
| 2-fluorohexahydro-1H- | 4-bromo-7-[[(2R,8S)-2-fluoro- | |
| pyrrolizin-7a- | 1,2,3,5,6,7-hexahydropyrrolizin- | |
| yl)methanol | 8-yl]methoxy]-9-[8-[2- | |
| | (methoxymethoxy)propyl]-3,8- | |
| | diazabicyclo[3.2.1]octan-3-yl]-2- | |
| | methyl-pyrazolo[4,3-f]quinazoline | |

### Intermediate 1-62, 5-[7-chloro-4-[8-ethyl-7-fluoro-3-(methoxymethoxy)-1-naphthyl]-2-methyl-pyrazolo[4,3-f]quinazolin-9-yl]-N,N-dimethyl-4,6,7,8-tetrahydropyrazolo[1,5-a][1,4]diazepine-2-carboxamide

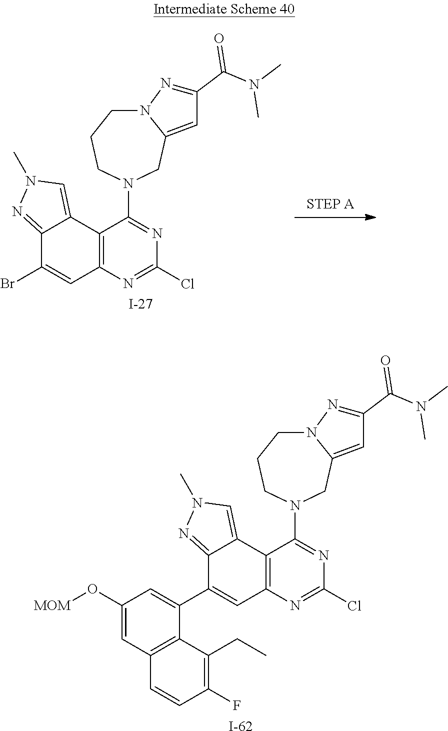

Step A, 5-[7-chloro-4-[8-ethyl-7-fluoro-3-(methoxymethoxy)-1-naphthyl]-2-methyl-pyrazolo[4,3-f]quinazolin-9-yl]-N, N-dimethyl-4,6,7,8-tetrahydropyrazolo[1,5-a][1,4]diazepine-2-carboxamide (1-62). A stirring mixture of 2-[8-ethyl-7-fluoro-3-(methoxymethoxy)-1-naphthyl]-4,4,5,5-tetramethyl-1,3,2-dioxaborolane (715.04 mg, 1.98 mmol), 5-(4-bromo-7-chloro-2-methyl-pyrazolo[4,3-f]quinazolin-9-yl)-N,N-dimethyl-4,6,7,8-tetrahydropyrazolo[1,5-a][1,4]diazepine-2-carboxamide (1. g 1.98 mmol) and Cesium Carbonate (1293.48 mg, 3.97 mmol) in 1,4-Dioxane (10 mL) and Water (2 mL) was degassed with nitrogen before the addition of [1,1′-Bis(di-tert-butylphosphino)ferrocene]dichloropalladium(II) (129.37 mg, 0.2 mmol). The reaction mixture was heated to 95° C. and left to stir overnight. The reaction mixture was left to cool to room temperature before being passed through phase separating filter paper and the solvents reduced in vacuo to afford the crude material. The crude material was purified by flash column chromatography (eluting in 0-100% EtOAc in Pet. ether) to afford the desired product 5-[7-chloro-4-[8-ethyl-7-fluoro-3-(methoxymethoxy)-1-naphthyl]-2-methyl-pyrazolo[4,3-f]quinazolin-9-yl]-N, N-dimethyl-4,6,7,8-tetrahydropyrazolo[1,5-a][1,4]diazepine-2-carboxamide (1.1 g, 1.1382 mmol, 57% yield) as a yellow solid

UPLC-MS (ES⁺, Method 5): 2.05 min, m/z 657.2 [M+H]⁺.

The intermediates in table 11 below were prepared by analogy with 1-62 (intermediate scheme 40) replacing building blocks for step A as described in the table entry.

TABLE 11

| | | UPLC-MS |
|---|---|---|
| Intermediate Building block | Intermediate Structure/Name | (ES⁺): min, m/z |
| Intermediate I-109 and 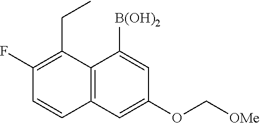 | Intermediate I-110 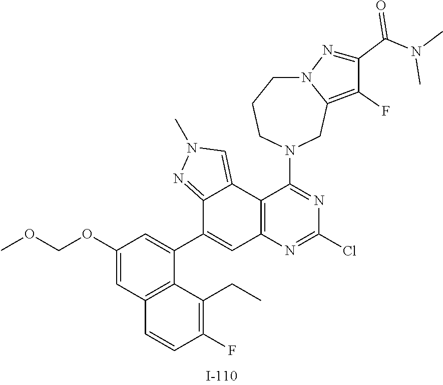 | 2.09 min, 677.2 [M + H]⁺ (Method 5) |
| [8-ethyl-7-fluoro-3- | 5-[7-chloro-4-[8-ethyl-7-fluoro-3- | |
| (methoxymethoxy)naphthalen- | (methoxymethoxy)-1-naphthyl]-2- | |
| 1-yl]boronic acid | methyl-pyrazolo[4,3-f]quinazolin-9-yl]- | |
| | 3-fluoro-N,N-dimethyl-4,6,7,8- | |
| | tetrahydropyrazolo[1,5- | |
| | a][1,4]diazepine-2-carboxamide | |

### Intermediate I-70, 7,9-dichloro-4-[8-ethyl-7-fluoro-3-(methoxymethoxy)-1-naphthyl]-2-methyl-pyrazolo[4,3-f]quinazoline

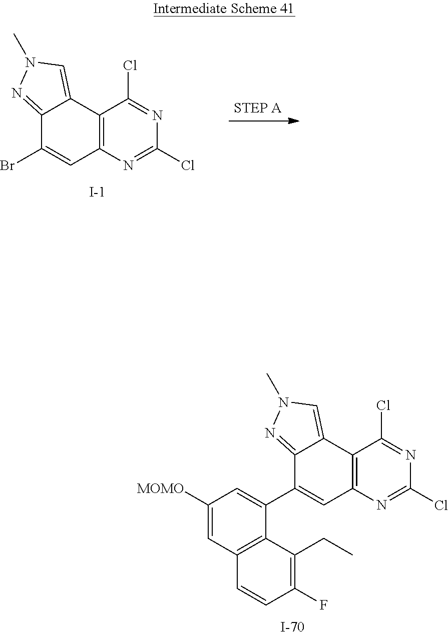

Step A, 7,9-dichloro-4-[8-ethyl-7-fluoro-3-(methoxymethoxy)-1-naphthyl]-2-methyl-pyrazolo[4,3-f]quinazoline (1-70). A solution of 4-bromo-7,9-dichloro-2-methyl-pyrazolo[4,3-f]quinazoline (100. mg, 0.3 mmol), 2-[8-ethyl-7-fluoro-3-(methoxymethoxy)-1-naphthyl]-4,4,5,5-tetramethyl-1,3,2-dioxaborolane (108.51 mg, 0.3 mmol) and Cesium Carbonate (196.29 mg, 0.6 mmol) in 1,4-Dioxane (1.13 mL) and Water (0.37 mL) was degassed with nitrogen for 2 mins. [1,1′-Bis(di-tert-butylphosphino)ferrocene]dichloropalladium(II) (19.63 mg, 0.03 mmol) was added. Reaction mixture allowed to stir at 70° C. for 4 hrs. Reaction mixture cooled back down to room temperature. Diluted with EtOAc (10 mL) and filtered over a plug of celite. Plug washed with EtOAc (2×10 mL). Filtrate collected and concentrated under reduced pressure. Purification by flash column chromatography on silica gel eluting with 0-80% ethyl acetate in petroleum ether afforded 7,9-dichloro-4-[8-ethyl-7-fluoro-3-(methoxymethoxy)-1-naphthyl]-2-methyl-pyrazolo[4,3-f]quinazoline (77.5 mg, 0.1597 mmol, 53% yield) (1-70) as an off-white solid.

UPLC-MS (ES⁺, Method 5): 2.34 min, m/z 485.1 [M+H]⁺.

¹H NMR (400 MHz, CDCl₃) δ/ppm: 8.77 (s, 1H), 7.73 (s, 1H), 7.70 (dd, J=9.0, 5.9 Hz, 1H), 7.53 (d, J=2.7 Hz, 1H), 7.29-7.23 (m, 1H), 7.19 (d, J=2.6 Hz, 1H), 5.32-5.26 (m, 2H), 4.21 (s, 3H), 3.52 (s, 3H), 2.54-2.41 (m, 1H), 2.15-2.02 (m, 1H), 0.65 (t, J=7.4 Hz, 3H).

### Intermediate I-71, 7-chloro-4-[8-ethyl-7-fluoro-3-(methoxymethoxy)-1-naphthyl]-2-methyl-9-(1-methylpyrazol-3-yl)pyrazolo[4,3-f]quinazoline

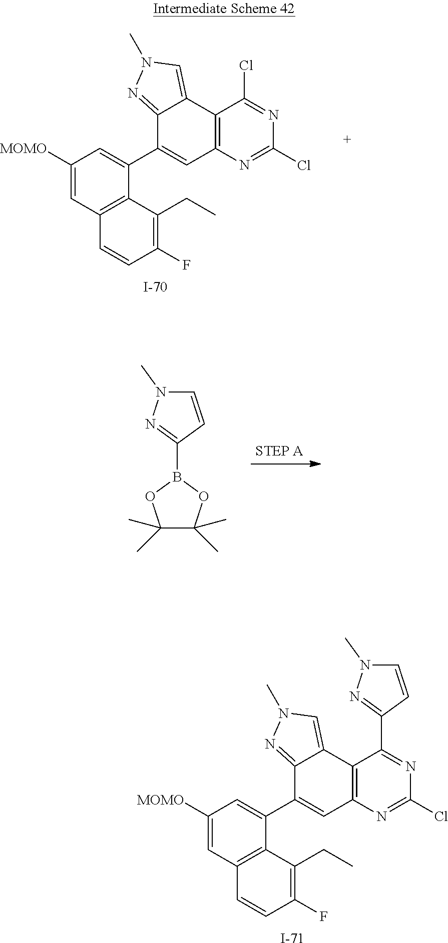

Step A, 7-chloro-4-[8-ethyl-7-fluoro-3-(methoxymethoxy)-1-naphthyl]-2-methyl-9-(1-methylpyrazol-3-yl)pyrazolo[4,3-f]quinazoline (1-71). A solution of 7,9-dichloro-4-[8-ethyl-7-fluoro-3-(methoxymethoxy)-1-naphthyl]-2-methyl-pyrazolo[4,3-f]quinazoline (193.5 mg, 0.4 mmol), 1-Methyl-3-(4,4,5,5-tetramethyl-1,3,2-dioxaborolan-2-yl)-1H-pyrazole (99.55 mg, 0.48 mmol) and Potassium carbonate (110.2 mg, 0.8 mmol) in 1,4-Dioxane (2.9902 mL) and Water (0.9967 mL) was degassed with nitrogen for 2 min. [1,1′-Bis(di-tert-butylphosphino)ferrocene]dichloropalladium(II) (25.98 mg, 0.04 mmol) was added. Reaction mixture was stirred at 70° C. for 2 hrs. Reaction mixture allowed to cool back down to room temperature. Partitioned between a layer of EtOAc (20 mL) and water (20 mL). Organic layer separated and aqueous layer extracted with EtOAc (20 mL). Organic layers combined, washed with a saturated solution of brine (10 mL), dried over sodium sulfate, filtered and concentrated under reduced pressure. Purification by flash column chromatography on silica gel eluting with 0-50% ethyl acetate in petrol afforded 7-chloro-4-[8-ethyl-7-fluoro-3-(methoxymethoxy)-1-naphthyl]-2-methyl-9-(1-methylpyrazol-3-yl)pyrazolo[4,3-f]quinazoline (69.9 mg, 0.1316 mmol, 33% yield) (1-71) as an off-white solid.

¹H NMR (400 MHz, DMSO-d6) δ/ppm: 7.91 (dd, J=9.1, 6.0 Hz, 1H), 7.82 (s, 1H), 7.79 (d, J=2.0 Hz, 1H), 7.67 (d, J=2.7 Hz, 1H), 7.54 (s, 1H), 7.43 (t, J=9.4 Hz, 1H), 7.18 (d, J=2.6 Hz, 1H), 6.87 (d, J=2.0 Hz, 1H), 5.38-5.31 (m, 2H), 4.00 (s, 3H), 3.79 (s, 3H), 3.44 (s, 3H), 2.54-2.38 (m, 1H), 2.14-1.98 (m, 1H), 0.60 (t, J=7.3 Hz, 3H).

UPLC-MS (ES⁺, Method 2): 2.05 min, m/z 531.4 [M+H]⁺.

The intermediates in table 12 below were prepared by analogy with I-71 (intermediate scheme 42) replacing building block 1-Methyl-3-(4,4,5,5-tetramethyl-1,3,2-dioxaborolan-2-yl)-1H-pyrazole as described in the table entry.

TABLE 12

| | | UPLC-MS |
|---|---|---|
| Intermediate Building block | Intermediate Structure/Name | (ES⁺): min, m/z |
| Intermediate I-70 and 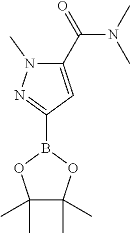 | Intermediate 1-80 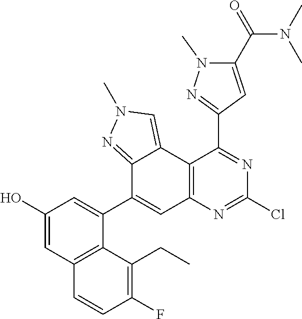 | 2.18 min, 604.2 [M + H]⁺ (Method 5) |
| A-25 | I-80 | |
| N,N,2-trimethyl-5-(4,4,5,5- | 5-[7-chloro-4-[8-ethyl-7- | |
| tetramethyl-1,3,2- | fluoro-3-(methoxymethoxy)- | |
| dioxaborolan-2-yl)pyrazole- | 1-naphthyl]-2-methyl- | |
| 3-carboxamide (A-25). | pyrazolo[4,3-f]quinazolin-9- | |
| | yl]-N,N,2-trimethyl-pyrazole- | |
| | 3-carboxamide | |

### Intermediate 1-95, 7-chloro-4-[8-ethyl-7-fluoro-3-(methoxymethoxy)-1-naphthyl]-9-[2-(methoxymethoxymethyl)-4,6,7,8-tetrahydropyrazolo[1,5-a][1,4]diazepin-5-yl]-2-methyl-pyrazolo[4,3-f]quinazoline

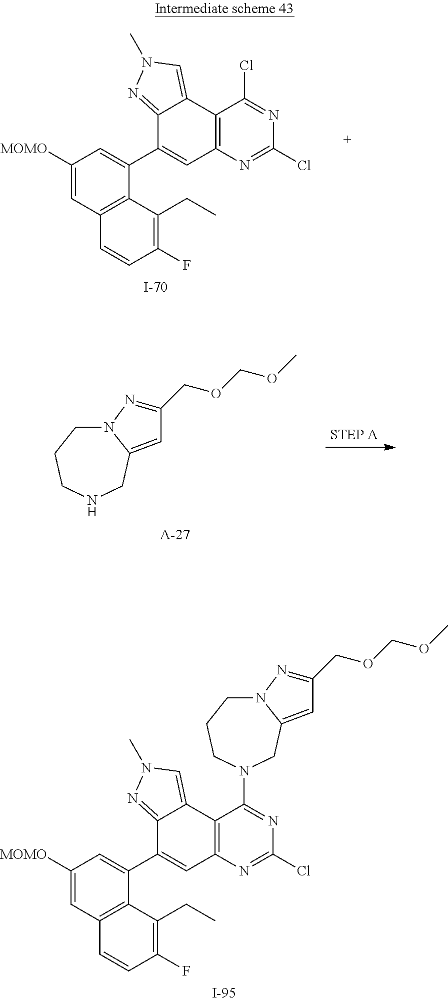

Step A, 7-chloro-4-[8-ethyl-7-fluoro-3-(methoxymethoxy)-1-naphthyl]-9-[2-(methoxymethoxymethyl)-4,6,7,8-tetrahydropyrazolo[1,5-a][1,4]diazepin-5-yl]-2-methyl-pyrazolo[4,3-f]quinazoline (1-95). To a stirred solution of 7,9-dichloro-4-[8-ethyl-7-fluoro-3-(methoxymethoxy)-1-naphthyl]-2-methyl-pyrazolo[4,3-f]quinazoline (115 mg, 0.24 mmol) and 2-(methoxymethoxymethyl)-5,6,7,8-tetrahydro-4H-pyrazolo[1,5-a][1,4]diazepine (55.06 mg, 0.26 mmol) (A-27) in DCM (2 mL) was added N, N-diisopropylethylamine (0.12 mL, 0.71 mmol). The reaction was stirred overnight at room temperature. The reaction was then stirred at 50° C. for 5 hrs. The reaction was partitioned between EtOAc and DCM and the layers separated. The aqueous layer was extracted with DCM (2×). The combined organic extracts were dried via phase separator and evaporated to afford a yellow solid. This material was purified via silica gel chromatography eluting 0-100% EtOAc in petroleum ether, then 0-20% MeOH in DCM to afford 7-chloro-4-[8-ethyl-7-fluoro-3-(methoxymethoxy)-1-naphthyl]-9-[2-(methoxymethoxymethyl)-4,6,7,8-tetrahydropyrazolo[1,5-a][1,4]diazepin-5-yl]-2-methyl-pyrazolo[4,3-f]quinazoline (130 mg, 0.197 mmol, 83% yield) as a yellow gum.

UPLC-MS (ES⁺, Method 5): 2.15 min, m/z 660.2 [M+H]⁺.

### Intermediate 1-97, (3R)-1-(4-bromo-7-ethylsulfonyl-2,5-dimethyl-pyrazolo[4,3-f]quinazolin-9-yl)-3-methyl-piperidin-3-ol

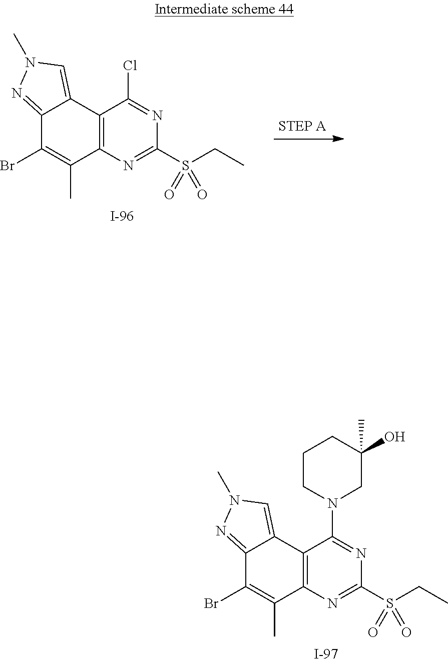

Step A, (3S)-1-(4-bromo-7-ethylsulfonyl-2,5-dimethyl-pyrazolo[4,3-f]quinazolin-9-yl)-3-methyl-piperidin-3-ol (I-97). N,N-Diisopropylethylamine (0.3 mL, 1.71 mmol) was added to a stirring solution of 4-bromo-9-chloro-7-ethylsulfonyl-2,5-dimethyl-pyrazolo[4,3-f]quinazoline (230. mg, 0.57 mmol) and (R)-3-Methylpiperidin-3-ol hydrochloride (86.4 mg, 0.57 mmol) in DCM (2.97 mL) and DMF (0.74 mL). The reaction mixture was left to stir at 25° C. for 4 hrs. To the reaction was added water and DCM. The phases were separated. The aqueous phase was extracted with DCM (3×25 ml). The combined organics were dried using a phase separating filter and concentrated to afford (3S)-1-(4-bromo-7-ethylsulfonyl-2,5-dimethyl-pyrazolo[4,3-f]quinazolin-9-yl)-3-methyl-piperidin-3-ol (0.274 g, 0.568 mmol, 100% yield) as a yellow solid.

UPLC-MS (ES⁺, Method 5): 1.71 min, m/z 484.0 [M+H]⁺.

The following examples in table 13 were made by analogy with I-97 (Intermediate scheme 44) starting from the intermediate and building blocks for step A as described in the table.

TABLE 13

| | | UPLC-MS |
|---|---|---|
| Intermediate Building block | Intermediate Structure/Name | (ES⁺): min, m/z |
| I-96 and 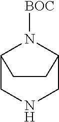 [SMILES: CC(C)(C)OC(=O)N1C2CCC1CNC2] | Intermediate I-99 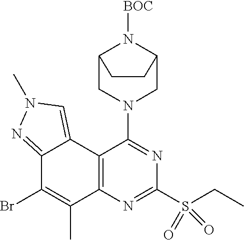 [SMILES: CCS(=O)(=O)c1nc(N2CC3CCC(C2)N3C(=O)OC(C)(C)C)c2c(n1)c(C)c(Br)c1nn(C)cc12] | 2.04 min, 581.0 [M + H]⁺ (Method 5) |
| 8-Boc-3,8- | I-99 | |
| diazabicyclo[3.2.1]octane | tert-butyl 3-(4-bromo-7- | |
| | ethylsulfonyl-2,5-dimethyl- | |
| | pyrazolo[4,3-f]quinazolin-9-yl)- | |
| | 3,8-diazabicyclo[3.2.1]octane- | |
| | 8-carboxylate | |
| I-96 and 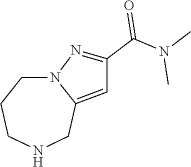 | Intermediate I-101 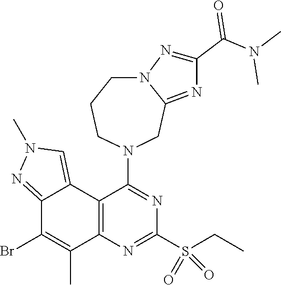 [SMILES: CCS(=O)(=O)c1nc(N2CCCn3nc(C(=O)N(C)C)nc3C2)c2c(n1)c(C)c(Br)c1nn(C)cc12] | 1.63 min, 577.0 [M + H]⁺ (Method 5) |
| (A-7) | I-101 | |
| N,N-dimethyl-5,6,7,8- | 5-(4-bromo-7-ethylsulfonyl- | |
| tetrahydro-4H-pyrazolo[1,5- | 2,5-dimethyl-pyrazolo[4,3- | |
| a][1,4]diazepine-2- | f]quinazolin-9-yl)-N,N- | |
| carboxamide; hydrochloride | dimethyl-4,6,7,8- | |
| (A-7) | tetrahydropyrazolo[1,5- | |
| | a][1,4]diazepine-2- | |
| | carboxamide | |
| I-96 and 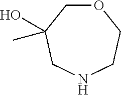 | Intermediate I-115 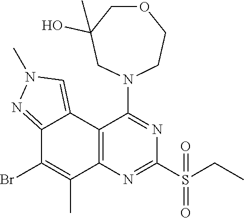 [SMILES: CCS(=O)(=O)c1nc(N2CCOCC(C)(O)C2)c2c(n1)c(C)c(Br)c1nn(C)cc12] | 1.64 min, 499.9 [M + H]⁺ (Method 5) |
| 6-Methyl-1,4-oxazepan-6- | I-115 | |
| ol•HCl | 4-(4-bromo-7-ethylsulfonyl- | |
| | 2,5-dimethyl-pyrazolo[4,3- | |
| | f]quinazolin-9-yl)-6-methyl-1,4- | |
| | oxazepan-6-ol | |
| I-124 and 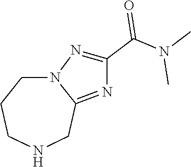 | Intermediate I-125 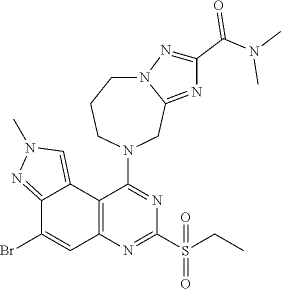 [SMILES: CCS(=O)(=O)c1nc(N2CCCn3nc(C(=O)N(C)C)nc3C2)c2c(cc(Br)c3nn(C)cc32)n1] | 1.43 min, 562.0/564.0 [M + H]⁺ (Method 5) |
| A-35 | I-125 | |
| N,N-dimethyl-6,7,8,9- | 8-(4-bromo-7-ethylsulfonyl-2- | |
| tetrahydro-5H- | methyl-pyrazolo[4,3- | |
| [1,2,4]triazolo[1,5- | f]quinazolin-9-yl)-N,N- | |
| a][1,4]diazepine-2- | dimethyl-5,6,7,9-tetrahydro- | |
| carboxamide (A-35) | [1,2,4]triazolo[1,5- | |
| | a][1,4]diazepine-2- | |
| | carboxamide | |
| I-124 and 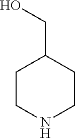 | Intermediate I-127 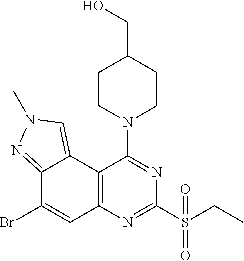 [SMILES: CCS(=O)(=O)c1nc(N2CCC(CO)CC2)c2c(cc(Br)c3nn(C)cc32)n1] | 1.54 min, 467.9/469.9 [M + H]⁺ (Method 5) |
| 4-Piperidinemethanol | I-127 | |
| | [1-(4-bromo-7-ethylsulfonyl-2- | |
| | methyl-pyrazolo[4,3- | |
| | f]quinazolin-9-yl)-4- | |
| | piperidyl]methanol | |
| I-124 and 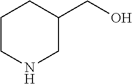 | Intermediate I-129 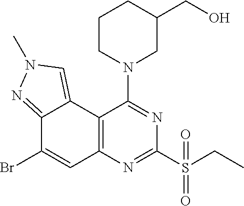 [SMILES: CCS(=O)(=O)c1nc(N2CCCC(CO)C2)c2c(cc(Br)c3nn(C)cc32)n1] | 1.57 min, 467.9/469.9 [M + H]⁺ (Method 5) |
| 3-piperidinemethanol | I-129 | |
| | [1-(4-bromo-7-ethylsulfonyl-2- | |
| | methyl-pyrazolo[4,3- | |
| | f]quinazolin-9-yl)-3- | |
| | piperidyl]methanol | |
| I-124 and 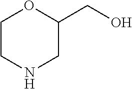 | Intermediate I-131 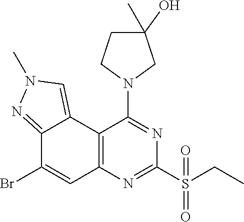 | 1.45 min, 469.9/471.9 [M + H]⁺ (Method 5) |
| morpholin-2-ylmethanol | I-131 | |
| | [4-(4-bromo-7-ethylsulfonyl-2- | |
| | methyl-pyrazolo[4,3- | |
| | f]quinazolin-9-yl)morpholin-2- | |
| | yl]methanol | |
| I-124 and 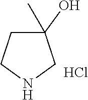 | Intermediate I-133 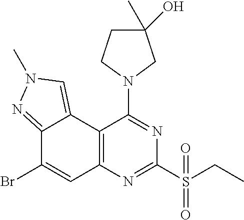 [SMILES: CCS(=O)(=O)c1nc(N2CCC(C)(O)C2)c2c(cc(Br)c3nn(C)cc32)n1] | 1.36 min, 454.0/456.0 [M + H]⁺ (Method 2) |
| 3-methylpyrrolidin-3- | I-133 | |
| ol; hydrochloride | 1-(4-bromo-7-ethylsulfonyl-2- | |
| | methyl-pyrazolo[4,3- | |
| | f]quinazolin-9-yl)-3-methyl- | |
| | pyrrolidin-3-ol | |
| I-124 and 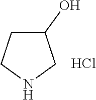 | Intermediate I-135 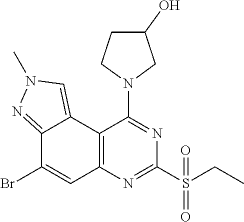 [SMILES: CCS(=O)(=O)c1nc(N2CCC(O)C2)c2c(cc(Br)c3nn(C)cc32)n1] | 1.30 min, 440.0/442.0 [M + H]⁺ (Method 2) |
| pyrrolidin-3-ol; hydrochloride | I-135 | |
| | 1-(4-bromo-7-ethylsulfonyl-2- | |
| | methyl-pyrazolo[4,3- | |
| | f]quinazolin-9-yl)pyrrolidin-3-ol | |
| Intermediate I-117a and 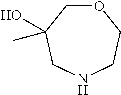 | Intermediate I-139 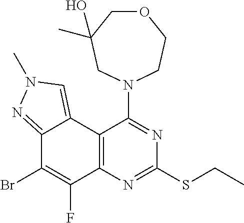 [SMILES: CCSc1nc(N2CCOCC(C)(O)C2)c2c(n1)c(F)c(Br)c1nn(C)cc12] | 1.74 min, 470.1/472.1 [M + H]⁺ (Method 2) |
| 6-Methyl-1,4-oxazepan-6-ol | I-139 | |
| | 4-(4-bromo-7-ethylsulfanyl-5- | |
| | fluoro-2-methyl-pyrazolo[4,3- | |
| | f]quinazolin-9-yl)-6-methyl-1,4- | |
| | oxazepan-6-ol | |
| Intermediate I-117a and 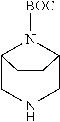 [SMILES: CC(C)(C)OC(=O)N1C2CCC1CNC2] | Intermediate I-141 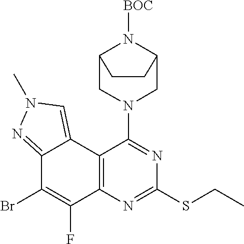 [SMILES: CCSc1nc(N2CC3CCC(C2)N3C(=O)OC(C)(C)C)c2c(n1)c(F)c(Br)c1nn(C)cc12] | 2.19 min, 551.2/553.2 [M + H]⁺ (Method 2) |
| 8-Boc-3,8- | I-141 | |
| diazabicyclo[3.2.1]octane | tert-butyl 3-(4-bromo-7- | |
| | ethylsulfanyl-5-fluoro-2- | |
| | methyl-pyrazolo[4,3- | |
| | f]quinazolin-9-yl)-3,8- | |
| | diazabicyclo[3.2.1]octane-8- | |
| | carboxylate | |
| Intermediate I-117a and 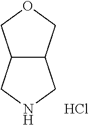 | Intermediate I-144 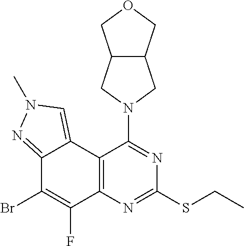 [SMILES: CCSc1nc(N2CC3COCC3C2)c2c(n1)c(F)c(Br)c1nn(C)cc12] | 1.72 min, 452.0/454.0 [M + H]⁺ (Method 2) |
| hexahydro-1H-furo[3,4- | I-144 | |
| c]pyrrole hydrochloride | 5-(4-bromo-7-ethylsulfanyl-5- | |
| | fluoro-2-methyl-pyrazolo[4,3- | |
| | f]quinazolin-9-yl)- | |
| | 1,3,3a,4,6,6a- | |
| | hexahydrofuro[3,4-c]pyrrole | |
| Intermediate I-117a and 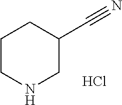 | Intermediate I-147 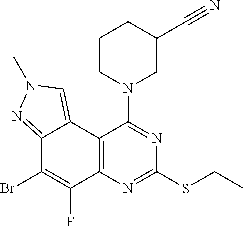 [SMILES: CCSc1nc(N2CCCC(C#N)C2)c2c(n1)c(F)c(Br)c1nn(C)cc12] | 1.91 min, 449.1/451.1 [M + H]⁺ (Method 2) |
| Piperidine-3-carbonitrile | I-147 | |
| hydrochloride | 1-(4-bromo-7-ethylsulfanyl-5- | |
| | fluoro-2-methyl-pyrazolo[4,3- | |
| | f]quinazolin-9-yl)piperidine-3- | |
| | carbonitrile | |
| Intermediate I-117a and 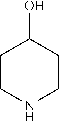 | Intermediate I-149 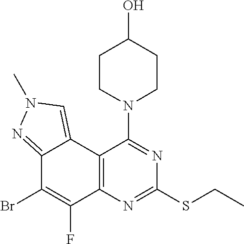 [SMILES: CCSc1nc(N2CCC(O)CC2)c2c(n1)c(F)c(Br)c1nn(C)cc12] | 1.71 min, 439.9/441.9 [M + H]⁺ (Method 2) |
| Piperidin-4-ol | I-149 | |
| | 1-(4-bromo-7-ethylsulfanyl-5- | |
| | fluoro-2-methyl-pyrazolo[4,3- | |
| | f]quinazolin-9-yl)piperidin-4-ol | |
| Intermediate I-117a and 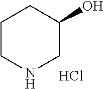 | Intermediate I-151 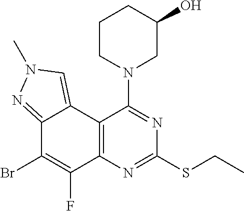 [SMILES: CCSc1nc(N2CCC[C@@H](O)C2)c2c(n1)c(F)c(Br)c1nn(C)cc12] | 1.75 min, 440.0/442.0 [M + H]⁺ (Method 2) |
| (R)-(+)-3-Hydroxypiperidine | I-151 | |
| hydrochloride | (3R)-1-(4-bromo-7- | |
| | ethylsulfanyl-5-fluoro-2- | |
| | methyl-pyrazolo[4,3- | |
| | f]quinazolin-9-yl)piperidin-3-ol | |
| Intermediate I-117a and 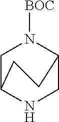 [SMILES: CC(C)(C)OC(=O)N1CC2CCC1CN2] | Intermediate I-153 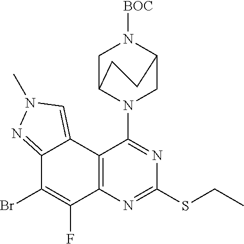 [SMILES: CCSc1nc(N2CC3CCC2CN3C(=O)OC(C)(C)C)c2c(n1)c(F)c(Br)c1nn(C)cc12] | 2.28 min, 551.0/553.0 [M + H]⁺ (Method 5) |
| tert-Butyl 2,5- | I-153 | |
| Diazabicyclo[2.2.2]octane- | tert-butyl 5-(4-bromo-7- | |
| 2-carboxylate | ethylsulfanyl-5-fluoro-2- | |
| | methyl-pyrazolo[4,3- | |
| | f]quinazolin-9-yl)-2,5- | |
| | diazabicyclo[2.2.2]octane-2- | |
| | carboxylate | |
| Intermediate I-96 and 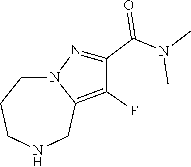 | Intermediate I-155 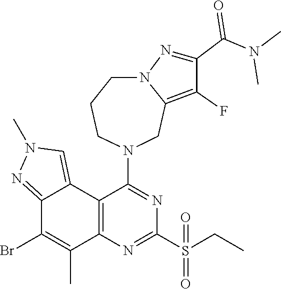 [SMILES: CCS(=O)(=O)c1nc(N2CCCn3nc(C(=O)N(C)C)c(F)c3C2)c2c(n1)c(C)c(Br)c1nn(C)cc12] | 1.69 min, 593.0/595.0 [M + H]⁺ (Method 5) |
| A-29 | I-155 | |
| 3-fluoro-N,N-dimethyl- | 5-(4-bromo-7-ethylsulfonyl- | |
| 5,6,7,8-tetrahydro-4H- | 2,5-dimethyl-pyrazolo[4,3- | |
| pyrazolo[1,5- | f]quinazolin-9-yl)-3-fluoro-N,N- | |
| a][1,4]diazepine-2- | dimethyl-4,6,7,8- | |
| carboxamide. | tetrahydropyrazolo[1,5- | |
| | a][1,4]diazepine-2- | |
| | carboxamide | |
| Intermediate I-96 and 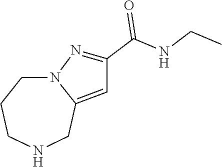 | Intermediate I-157 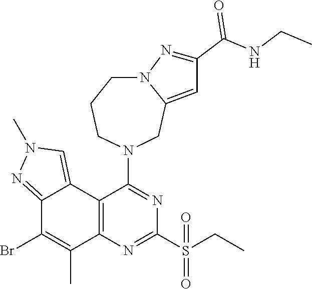 [SMILES: CCNC(=O)c1cc2n(n1)CCCN(c1nc(S(=O)(=O)CC)nc3c(C)c(Br)c4nn(C)cc4c13)C2] | 1.63 min, 575.0/577.0 [M + H]⁺ (Method 5) |
| A-6 | I-157 | |
| N-ethyl-5,6,7,8-tetrahydro- | 5-(4-bromo-7-ethylsulfonyl- | |
| 4H-pyrazolo[1,5- | 2,5-dimethyl-pyrazolo[4,3- | |
| a][1,4]diazepine-2- | f]quinazolin-9-yl)-N-ethyl- | |
| carboxamide | 4,6,7,8- | |
| | tetrahydropyrazolo[1,5- | |
| | a][1,4]diazepine-2- | |
| | carboxamide | |
| Intermediate I-96 and 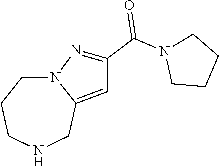 [SMILES: O=C(c1cc2n(n1)CCCNC2)N1CCCC1] | Intermediate I-159 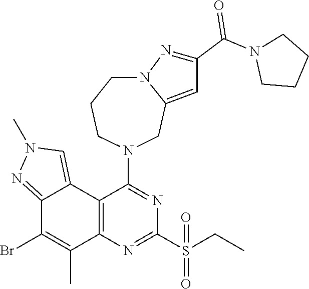 [SMILES: CCS(=O)(=O)c1nc(N2CCCn3nc(C(=O)N4CCCC4)cc3C2)c2c(n1)c(C)c(Br)c1nn(C)cc12] | 1.70 min, 601.1/603.3 [M + H]⁺ (Method 5) |
| A-8 | I-159 | |
| pyrrolidin-1-yl(5,6,7,8- | [5-(4-bromo-7-ethylsulfonyl- | |
| tetrahydro-4H-pyrazolo[1,5- | 2,5-dimethyl-pyrazolo[4,3- | |
| a][1,4]diazepin-2- | f]quinazolin-9-yl)-4,6,7,8- | |
| yl)methanone | tetrahydropyrazolo[1,5- | |
| | a][1,4]diazepin-2-yl]-pyrrolidin- | |
| | 1-yl-methanone | |
| Intermediate I-96a and 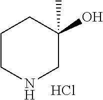 | Intermediate I-161 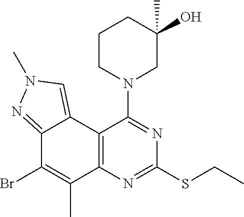 [SMILES: CCSc1nc(N2CCC[C@@](C)(O)C2)c2c(n1)c(C)c(Br)c1nn(C)cc12] | 2.23 min, 450.0/452.0 [M + H]⁺ (Method 5) |
| (R)-3-Methylpiperidin-3-ol | I-161 | |
| hydrochloride | (3R)-1-(4-bromo-7- | |
| | ethylsulfanyl-2,5-dimethyl- | |
| | pyrazolo[4,3-f]quinazolin-9-yl)- | |
| | 3-methyl-piperidin-3-ol | |
| Intermediate I-117a and 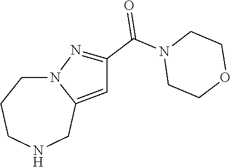 [SMILES: O=C(c1cc2n(n1)CCCNC2)N1CCOCC1] | Intermediate I-166 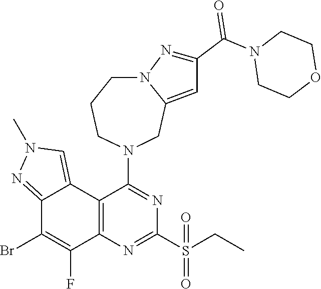 [SMILES: CCS(=O)(=O)c1nc(N2CCCn3nc(C(=O)N4CCOCC4)cc3C2)c2c(n1)c(F)c(Br)c1nn(C)cc12] | 1.40 min, 621.2/623.1 [M + H]⁺ (Method 2) |
| A-9 | I-166 | |
| morpholino(5,6,7,8- | [5-(4-bromo-7-ethylsulfonyl-5- | |
| tetrahydro-4H-pyrazolo[1,5- | fluoro-2-methyl-pyrazolo[4,3- | |
| a][1,4]diazepin-2- | f]quinazolin-9-yl)-4,6,7,8- | |
| yl)methanone (A-9) | tetrahydropyrazolo[1,5- | |
| | a][1,4]diazepin-2-yl]- | |
| | morpholino-methanone | |
| Intermediate I-117a and 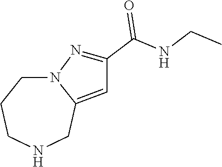 | Intermediate I-168 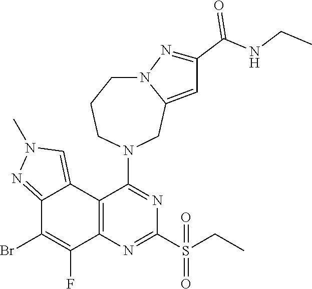 [SMILES: CCNC(=O)c1cc2n(n1)CCCN(c1nc(S(=O)(=O)CC)nc3c(F)c(Br)c4nn(C)cc4c13)C2] | 1.42 min, 579.2/581.1 [M + H]⁺ (Method 2) |
| A-6 | I-168 | |
| N-ethyl-5,6,7,8-tetrahydro- | 5-(4-bromo-7-ethylsulfonyl-5- | |
| 4H-pyrazolo[1,5- | fluoro-2-methyl-pyrazolo[4,3- | |
| a][1,4]diazepine-2- | f]quinazolin-9-yl)-N-ethyl- | |
| carboxamide | 4,6,7,8- | |
| | tetrahydropyrazolo[1,5- | |
| | a][1,4]diazepine-2- | |
| | carboxamide | |
| Intermediate I-117a and 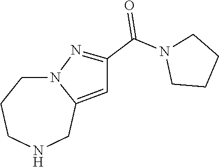 [SMILES: O=C(c1cc2n(n1)CCCNC2)N1CCCC1] | Intermediate I-170 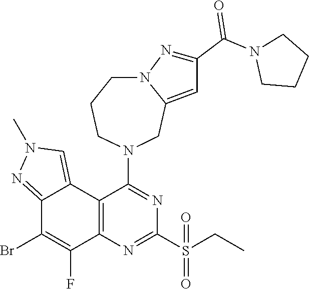 [SMILES: CCS(=O)(=O)c1nc(N2CCCn3nc(C(=O)N4CCCC4)cc3C2)c2c(n1)c(F)c(Br)c1nn(C)cc12] | 1.61 min, 605.1/607.1 [M + H]⁺ (Method 2) |
| A-8 | I-170 | |
| pyrrolidin-1-yl(5,6,7,8- | [5-(4-bromo-7-ethylsulfonyl-5- | |
| tetrahydro-4H-pyrazolo[1,5- | fluoro-2-methyl-pyrazolo[4,3- | |
| a][1,4]diazepin-2- | f]quinazolin-9-yl)-4,6,7,8- | |
| yl)methanone | tetrahydropyrazolo[1,5- | |
| | a][1,4]diazepin-2-yl]-pyrrolidin- | |
| | 1-yl-methanone | |
| Intermediate I-96a and 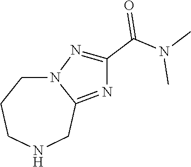 | Intermediate I-172 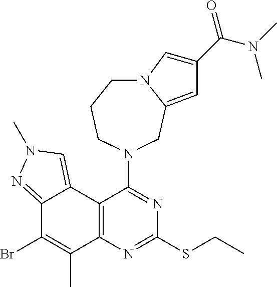 [SMILES: CCSc1nc(N2CCCn3cc(C(=O)N(C)C)cc3C2)c2c(n1)c(C)c(Br)c1nn(C)cc12] | 1.89 min, 544.1/546.1 [M + H]⁺ (Method 5) |
| A-35 | I-172 | |
| N,N-dimethyl-6,7,8,9- | 8-(4-bromo-7-ethylsulfanyl- | |
| tetrahydro-5H- | 2,5-dimethyl-pyrazolo[4,3- | |
| [1,2,4]triazolo[1,5- | f]quinazolin-9-yl)-N,N- | |
| a][1,4]diazepine-2- | dimethyl-5,6,7,9-tetrahydro- | |
| carboxamide (A-35) | [1,2,4]triazolo[1,5- | |
| | a][1,4]diazepine-2- | |
| | carboxamide | |
| Intermediate I-124 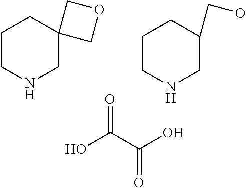 | Intermediate I-174 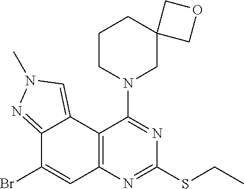 [SMILES: CCSc1nc(N2CCCC3(COC3)C2)c2c(cc(Br)c3nn(C)cc32)n1] | 1.93 min, 448.0/450.0 [M + H]⁺ (Method 5) |
| 2-Oxa-6- | I-174 | |
| azaspiro[3.5]nonane | | |
| hemioxalate | | |
| Intermediate I-117 and 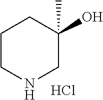 | Intermediate I-176 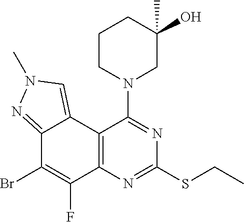 [SMILES: CCSc1nc(N2CCC[C@@](C)(O)C2)c2c(n1)c(F)c(Br)c1nn(C)cc12] | 1.97 min, 454.0/456.0 [M + H]⁺ (Method 5) |
| (R)-3-Methylpiperidin-3-ol | I-176 | |
| hydrochloride | (3R)-1-(4-bromo-7- | |
| | ethylsulfanyl-5-fluoro-2- | |
| | methyl-pyrazolo[4,3- | |
| | f]quinazolin-9-yl)-3-methyl- | |
| | piperidin-3-ol | |
| Intermediate I-117 and 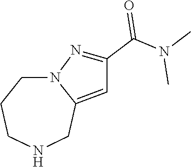 | Intermediate I-179 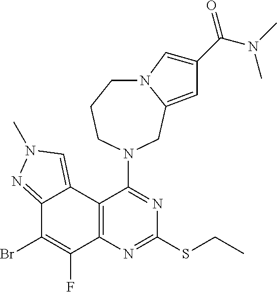 [SMILES: CCSc1nc(N2CCCn3cc(C(=O)N(C)C)cc3C2)c2c(n1)c(F)c(Br)c1nn(C)cc12] | 1.70 min, 547.1/549.1 [M + H]⁺ (Method 2) |
| A-7 | I-179 | |
| N,N-dimethyl-5,6,7,8- | 5-(4-bromo-7-ethylsulfanyl-5- | |
| tetrahydro-4H-pyrazolo[1,5- | fluoro-2-methyl-pyrazolo[4,3- | |
| a][1,4]diazepine-2- | f]quinazolin-9-yl)-N,N- | |
| carboxamide; hydrochloride | dimethyl-4,6,7,8- | |
| | tetrahydropyrazolo[1,5- | |
| | a][1,4]diazepine-2- | |
| | carboxamide | |
| Intermediate I-96a and 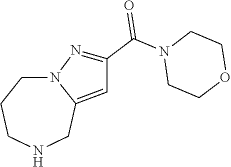 [SMILES: O=C(c1cc2n(n1)CCCNC2)N1CCOCC1] | Intermediate I-181 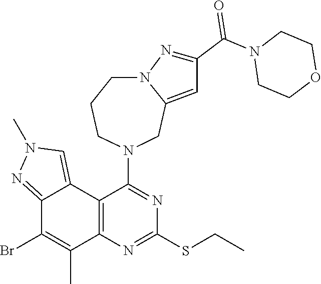 [SMILES: CCSc1nc(N2CCCn3nc(C(=O)N4CCOCC4)cc3C2)c2c(n1)c(C)c(Br)c1nn(C)cc12] | 2.02 min, 585.1/587.1 [M + H]⁺ (Method 5) |
| A-9 | I-181 | |
| morpholino(5,6,7,8- | [5-(4-bromo-7-ethylsulfanyl- | |
| tetrahydro-4H-pyrazolo[1,5- | 2,5-dimethyl-pyrazolo[4,3- | |
| a][1,4]diazepin-2- | f]quinazolin-9-yl)-4,6,7,8- | |
| yl)methanone (A-9) | tetrahydropyrazolo[1,5- | |
| | a][1,4]diazepin-2-yl]- | |
| | morpholino-methanone | |
| Intermediate 1-96a and 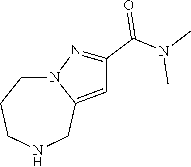 | Intermediate I-183 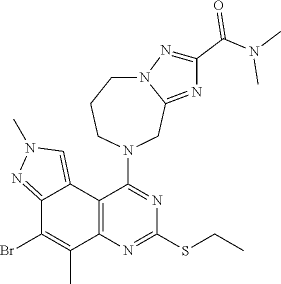 [SMILES: CCSc1nc(N2CCCn3nc(C(=O)N(C)C)nc3C2)c2c(n1)c(C)c(Br)c1nn(C)cc12] | 1.90 min, 545.2 [M + H]⁺ (Method 2) |
| A-7 | I-183 | |
| N,N-dimethyl-5,6,7,8- | 5-(4-bromo-7-ethylsulfanyl- | |
| tetrahydro-4H-pyrazolo[1,5- | 2,5-dimethyl-pyrazolo[4,3- | |
| a][1,4]diazepine-2- | f]quinazolin-9-yl)-N,N- | |
| carboxamide; hydrochloride | dimethyl-4,6,7,8- | |
| | tetrahydropyrazolo[1,5- | |
| | a][1,4]diazepine-2- | |
| | carboxamide | |
| Intermediate I-96a and 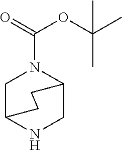 | Intermediate I-185 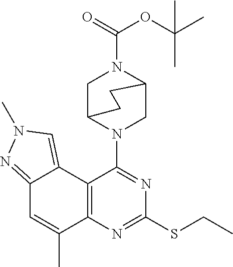 | 2.48 min, 547.1/549.1 [M + H]⁺ (Method 5) |
| tert-butyl 2,5- | I-185 | |
| diazabicyclo[2.2.2]octane-2- | tert-butyl 5-(4-bromo-7- | |
| carboxylate | ethylsulfanyl-2,5-dimethyl- | |
| | pyrazolo[4,3-f]quinazolin-9-yl)- | |
| | 2,5-diazabicyclo[2.2.2]octane- | |
| | 2-carboxylate | |
| Intermediate I-96a and 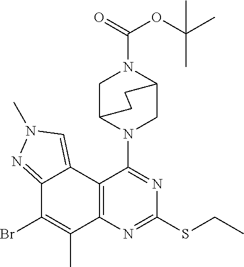 [SMILES: CCSc1nc(N2CC3CCC2CN3C(=O)OC(C)(C)C)c2c(n1)c(C)c(Br)c1nn(C)cc12] | Intermediate I-189 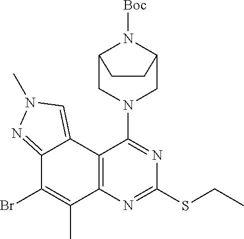 [SMILES: CCSc1nc(N2CC3CCC(C2)N3C(=O)OC(C)(C)C)c2c(n1)c(C)c(Br)c1nn(C)cc12] | 2.19 min, 553.2 [M + H]⁺ (Method 2) |
| 8-Boc-3,8- | I-189 | |
| diazabicyclo[3.2.1]octane | tert-butyl 3-(4-bromo-7- | |
| | ethylsulfanyl-5-fluoro-2- | |
| | methyl-pyrazolo[4,3- | |
| | f]quinazolin-9-yl)-3,8- | |
| | diazabicyclo[3.2.1]octane-8- | |
| | carboxylate | |
| Intermediate 1-117 and 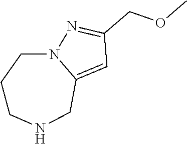 | Intermediate I-191 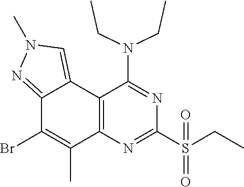 | 2.02 min, 702.3 [M + H]⁺ (Method 5) |
| A-36 | I-191 | |
| Intermediate A-36, 2- | 4-[8-ethyl-7-fluoro-3- | |
| (methoxymethyl)-5,6,7,8- | (methoxymethoxy)-1- | |
| tetrahydro-4H-pyrazolo[1,5- | naphthyl]-7-ethylsulfonyl-9-[2- | |
| a][1,4]diazepine | (methoxymethyl)-4,6,7,8- | |
| | tetrahydropyrazolo[1,5- | |
| | a][1,4]diazepin-5-yl]-2,5- | |
| | dimethyl-pyrazolo[4,3- | |
| | f]quinazoline | |
| Intermediate I-117a and 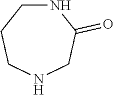 | Intermediate I-193 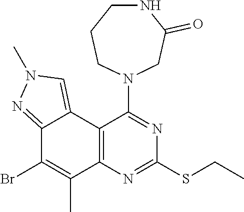 [SMILES: CCSc1nc(N2CCCNC(=O)C2)c2c(n1)c(C)c(Br)c1nn(C)cc12] | 1.46 min, 455.0 [M + 2H]⁺ (Method 2) |
| 1,4-diazepan-2-one | I-193 | |
| | 4-(4-bromo-7-ethylsulfanyl-5- | |
| | fluoro-2-methyl-pyrazolo[4,3- | |
| | f]quinazolin-9-yl)-1,4- | |
| | diazepan-2-one | |

### Intermediate I-118, (6S)-4-(4-bromo-7-ethylsulfonyl-5-fluoro-2-methyl-pyrazolo[4,3-f]quinazolin-9-yl)-6-methyl-1,4-oxazepan-6-ol

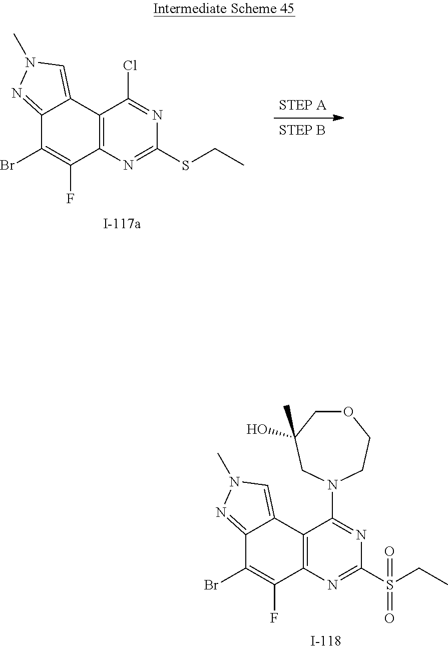

Step A, (6S)-4-(4-bromo-7-ethylsulfanyl-5-fluoro-2-methyl-pyrazolo[4,3-f]quinazolin-9-yl)-6-methyl-1,4-oxazepan-6-ol. To a solution of (6S)-6-methyl-1,4-oxazepan-6-ol;2,2,2-trifluoroacetic acid (210.51 mg, 0.86 mmol) and 4-bromo-9-chloro-7-ethylsulfanyl-5-fluoro-2-methyl-pyrazolo[4,3-f]quinazoline (1-117a) (129. mg, 0.34 mmol) in DCM (1.71 mL) was added N,N-Diisopropylethylamine (0.6 mL, 3.43 mmol) and the mixture stirred at room temperature for 30 min. Additional N,N-Diisopropylethylamine (0.3 mL, 1.72 mmol) was added. Allowed to stir at room temperature for 1 h. Partitioned between a layer of ethyl acetate (20 mL) and water (20 mL). Organic layer separated and aqueous layer extracted with ethyl acetate (20 mL). Organic layers combined, washed with a saturated solution of brine (10 mL), dried over sodium sulfate, filtered and concentrated under reduced pressure. Purification by flash column chromatography on silica gel eluting with 0-100% ethyl acetate in petrol followed by 0-5% MeOH in DCM to afford (6S)-4-(4-bromo-7-ethylsulfanyl-5-fluoro-2-methyl-pyrazolo[4,3-f]quinazolin-9-yl)-6-methyl-1,4-oxazepan-6-ol (123.2 mg, 0.2619 mmol, 76% yield) as a brown solid.

UPLC-MS (ES⁺, Method 5): 1.89 min, m/z 470.0/472.0 [M+H]⁺.

Step B, (6S)-4-(4-bromo-7-ethylsulfonyl-5-fluoro-2-methyl-pyrazolo[4,3-f]quinazolin-9-yl)-6-methyl-1,4-oxazepan-6-ol. At 0° C., m-chloroperbenzoic acid (146.75 mg, 0.65 mmol) was added to a solution of (6S)-4-(4-bromo-7-ethylsulfanyl-5-fluoro-2-methyl-pyrazolo[4,3-f]quinazolin-9-yl)-6-methyl-1,4-oxazepan-6-ol (123.2 mg, 0.26 mmol) in DCM (2.6193 mL). Afterwards, allowed to stir at room temperature for 1 h. Reaction mixture quenched with a saturated solution of sodium thiosulfate. Partitioned between a layer of ethyl acetate (20 mL) and water (20 mL). Organic layer separated, aqueous layer extracted with ethyl acetate (20 mL) and organic layers combined. Combined organic layers washed with a saturated solution of sodium bicarbonate (2×10 mL), a saturated solution of brine (10 mL), dried over sodium sulfate, filtered and concentrated under reduced pressure to afford (6S)-4-(4-bromo-7-ethylsulfonyl-5-fluoro-2-methyl-pyrazolo[4,3-f]quinazolin-9-yl)-6-methyl-1,4-oxazepan-6-ol (131.58 mg, 0.2619 mmol, 100% yield) as a clear oil.

UPLC-MS (ES⁺, Method 5): 1.55 min, m/z 501.9/503.9 [M+H]⁺.

The following examples in table 14 were made by analogy with I-118 (Intermediate scheme 45) starting from the intermediate and building blocks for step A as described in the table.

TABLE 14

| Intermediate Building block | Intermediate Structure/Name | UPLC-MS (ES⁺): min, m/z |
|---|---|---|
| Intermediate I-117a and 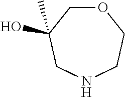 | Intermediate I-120 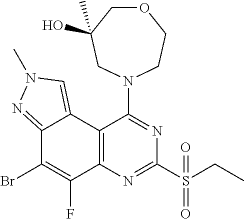 [SMILES: CCS(=O)(=O)c1nc(N2CCOC[C@](C)(O)C2)c2c(n1)c(F)c(Br)c1nn(C)cc12] | 1.41 min, 502.1/504.1 [M + H]⁺ (Method 2) |
| (6R)-6-methyl-1,4- | I-120 | |
| oxazepan-6-ol | (6R)-4-(4-bromo-7- | |
| | ethylsulfonyl-5-fluoro-2- | |
| | methyl-pyrazolo[4,3- | |
| | f]quinazolin-9-yl)-6-methyl- | |
| | 1,4-oxazepan-6-ol | |
| Intermediate I-117a and 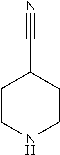 | Intermediate I-122 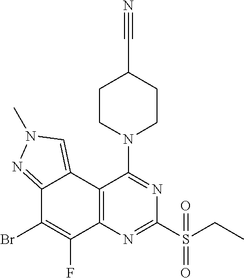 [SMILES: CCS(=O)(=O)c1nc(N2CCC(C#N)CC2)c2c(n1)c(F)c(Br)c1nn(C)cc12] | 1.56 min, 481.0/483.0 [M + H]⁺ (Method 2) |
| 4-Cyanopiperidine | I-122 | |
| | 1-(4-bromo-7-ethylsulfonyl- | |
| | 5-fluoro-2-methyl- | |
| | pyrazolo[4,3-f]quinazolin-9- | |
| | yl)piperidine-4-carbonitrile | |
| Intermediate 1-117a and 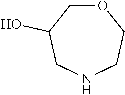 | Intermediate I-187 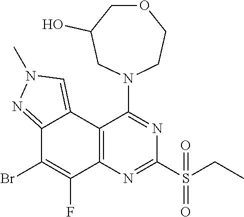 [SMILES: CCS(=O)(=O)c1nc(N2CCOCC(O)C2)c2c(n1)c(F)c(Br)c1nn(C)cc12] | 1.48 min, 487.9.1/489.9 [M + H]⁺ (Method 5) |
| 1,4-oxazepan-6-ol | I-187 | |
| | 4-(4-bromo-7-ethylsulfonyl- | |
| | 5-fluoro-2-methyl- | |
| | pyrazolo[4,3-f]quinazolin-9- | |
| | yl)-1,4-oxazepan-6-ol | |

### Intermediate 1-98, (3R)-1-[4-bromo-2,5-dimethyl-7-[[(2R,8S)-2-fluoro-1,2,3,5,6,7-hexahydropyrrolizin-8-yl]methoxy]pyrazolo[4,3-f]quinazolin-9-yl]-3-methyl-piperidin-3-ol

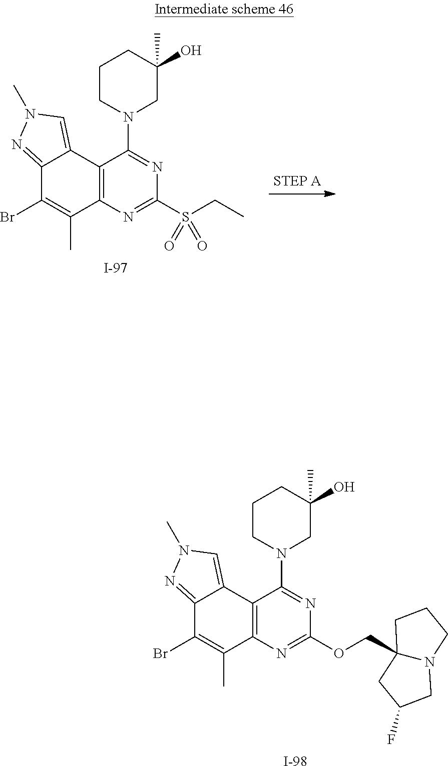

Step A, (3R)-1-[4-bromo-2,5-dimethyl-7-[[(2R,8S)-2-fluoro-1,2,3,5,6,7-hexahydropyrrolizin-8-yl]methoxy]pyrazolo[4,3-f]quinazolin-9-yl]-3-methyl-piperidin-3-ol (1-98). To a solution of ((2R,7aS)-2-Fluorohexahydro-1H-pyrrolizin-7a-yl) methanol (134.1 mg, 0.84 mmol) in THF (4.05 mL) under N₂ was added dropwise 1M Lithium bis(trimethylsilyl)amide in THF (0.78 mL, 0.78 mmol) at 0° C. The reaction mixture was stirred at this temperature for 20 mins then (3R)-1-(4-bromo-7-ethylsulfonyl-2,5-dimethyl-pyrazolo[4,3-f]quinazolin-9-yl)-3-methyl-piperidin-3-ol (250. mg, 0.52 mmol) in THF (1.1337 mL) was added in one portion. The reaction mixture was then stirred at 25° C. overnight. The reaction was quenched with saturated aqueous NaHCO₃, extracted with EtOAc, dried over magnesium sulfate, filtered and evaporated to give (3R)-1-[4-bromo-2,5-dimethyl-7-[[(2R,8S)-2-fluoro-1,2,3,5,6,7-hexahydropyrrolizin-8-yl]methoxy]pyrazolo[4,3-f]quinazolin-9-yl]-3-methyl-piperidin-3-ol (250 mg, 0.457 mmol, 88% yield) as a yellow oil. The material was used without further purification.

UPLC-MS (ES⁺, Method 5): 1.47 min, m/z 549.0 [M+H]⁺.

The following intermediates in table 15 were made by analogy with I-98 (Intermediate scheme 46) starting from the intermediate and building blocks for step A as described in the table.

TABLE 15

| Intermediate Building block | Intermediate Structure/Name | UPLC-MS (ES⁺): min, m/z |
|---|---|---|
| I-99 and 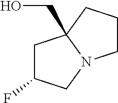 [SMILES: OC[C@@]12CCCN1C[C@H](F)C2] | Intermediate I-100 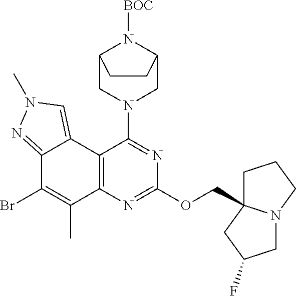 [SMILES: Cc1c(Br)c2nn(C)cc2c2c(N3CC4CCC(C3)N4C(=O)OC(C)(C)C)nc(OC[C@@]34CCCN3C[C@H](F)C4)nc12] | 1.74 min, 646.2 [M + H]⁺ (Method 5) |
| ((2R,7aS)-2- | I-100 | |
| fluorohexahydro-1H- | tert-butyl 3-[4-bromo-7- | |
| pyrrolizin-7a-yl)methanol | [[(2R,8S)-2-fluoro- | |
| | 1,2,3,5,6,7- | |
| | hexahydropyrrolizin-8- | |
| | yl]methoxy]-2,5-dimethyl- | |
| | pyrazolo[4,3-f]quinazolin-9- | |
| | yl]-3,8- | |
| | diazabicyclo[3.2.1]octane-8- | |
| | carboxylate | |
| I-101 and 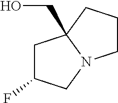 [SMILES: OC[C@@]12CCCN1C[C@H](F)C2] | Intermediate I-102 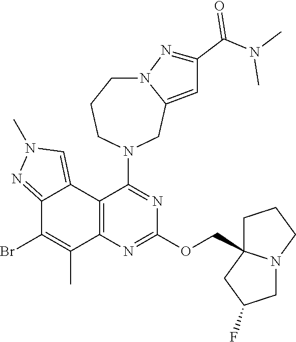 [SMILES: Cc1c(Br)c2nn(C)cc2c2c(N3CCCn4nc(C(=O)N(C)C)cc4C3)nc(OC[C@@]34CCCN3C[C@H](F)C4)nc12] | 1.42 min, 642.1 [M + H]⁺ (Method 5) |
| ((2R,7aS)-2- | I-102 | |
| fluorohexahydro-1H- | 5-[4-bromo-2,5-dimethyl-7- | |
| pyrrolizin-7a-yl)methanol | [[(2R,8S)-2-fluoro- | |
| | 1,2,3,5,6,7- | |
| | hexahydropyrrolizin-8- | |
| | yl]methoxy]pyrazolo[4,3- | |
| | f]quinazolin-9-yl]-N,N- | |
| | dimethyl-4,6,7,8- | |
| | tetrahydropyrazolo[1,5- | |
| | a][1,4]diazepine-2- | |
| | carboxamide | |
| I-115 and 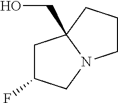 [SMILES: OC[C@@]12CCCN1C[C@H](F)C2] | Intermediate I-116 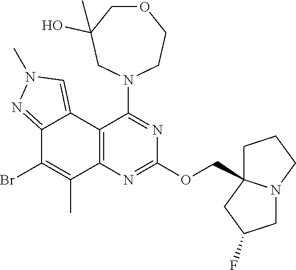 [SMILES: Cc1c(Br)c2nn(C)cc2c2c(N3CCOCC(C)(O)C3)nc(OC[C@@]34CCCN3C[C@H](F)C4)nc12] | 1.45 min, 565.0 [M + H]⁺ (Method 5) |
| ((2R,7aS)-2- | I-116 | |
| fluorohexahydro-1H- | 4-[4-bromo-2,5-dimethyl-7- | |
| pyrrolizin-7a-yl)methanol | [[(2R)-2-fluoro-1,2,3,5,6,7- | |
| | hexahydropyrrolizin-8- | |
| | yl]methoxy]pyrazolo[4,3- | |
| | f]quinazolin-9-yl]-6-methyl- | |
| | 1,4-oxazepan-6-ol | |
| I-118 and 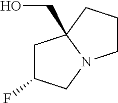 [SMILES: OC[C@@]12CCCN1C[C@H](F)C2] | Intermediate I-119 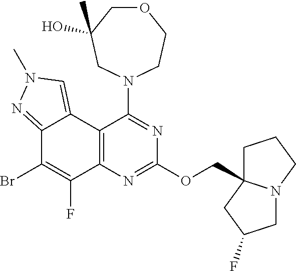 [SMILES: Cn1cc2c(n1)c(Br)c(F)c1nc(OC[C@@]34CCCN3C[C@H](F)C4)nc(N3CCOC[C@@](C)(O)C3)c12] | 1.37 min, 569.0 [M + H]⁺ (Method 5) |
| ((2R,7aS)-2- | I-119 | |
| fluorohexahydro-1H- | (6S)-4-[4-bromo-5-fluoro-2- | |
| pyrrolizin-7a-yl)methanol | methyl-7-[[(2R,8S)-2-fluoro- | |
| | 1,2,3,5,6,7- | |
| | hexahydropyrrolizin-8- | |
| | yl]methoxy]pyrazolo[4,3- | |
| | f]quinazolin-9-yl]-6-methyl- | |
| | 1,4-oxazepan-6-ol | |
| I-120 and 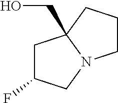 [SMILES: OC[C@@]12CCCN1C[C@H](F)C2] | Intermediate I-121 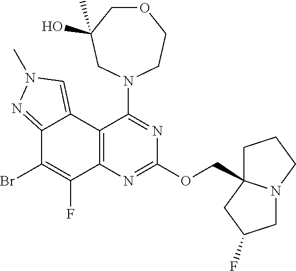 [SMILES: Cn1cc2c(n1)c(Br)c(F)c1nc(OC[C@@]34CCCN3C[C@H](F)C4)nc(N3CCOC[C@](C)(O)C3)c12] | 1.21 min, 569.2 [M + H]⁺ (Method 2) |
| ((2R,7aS)-2- | I-121 | |
| fluorohexahydro-1H- | (6R)-4-[4-bromo-5-fluoro-2- | |
| pyrrolizin-7a-yl)methanol | methyl-7-[[(2R,8S)-2-fluoro- | |
| | 1,2,3,5,6,7- | |
| | hexahydropyrrolizin-8- | |
| | yl]methoxy]pyrazolo[4,3- | |
| | f]quinazolin-9-yl]-6-methyl- | |
| | 1,4-oxazepan-6-ol | |
| I-122 and 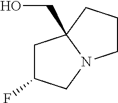 [SMILES: OC[C@@]12CCCN1C[C@H](F)C2] | Intermediate I-123 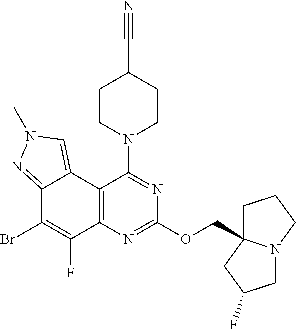 [SMILES: Cn1cc2c(n1)c(Br)c(F)c1nc(OC[C@@]34CCCN3C[C@H](F)C4)nc(N3CCC(C#N)CC3)c12] | 1.29 min, 546.1/548.1 [M + H]⁺ (Method 2) |
| ((2R,7aS)-2- | I-123 | |
| fluorohexahydro-1H- | 1-[4-bromo-5-fluoro-2- | |
| pyrrolizin-7a-yl)methanol | methyl-7-[[(2R,8S)-2-fluoro- | |
| | 1,2,3,5,6,7- | |
| | hexahydropyrrolizin-8- | |
| | yl]methoxy]pyrazolo[4,3- | |
| | f]quinazolin-9-yl]piperidine- | |
| | 4-carbonitrile | |
| I-125 and 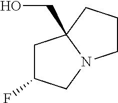 [SMILES: OC[C@@]12CCCN1C[C@H](F)C2] | Intermediate I-126 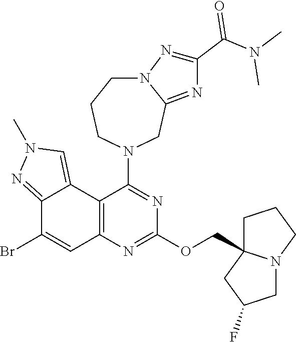 [SMILES: CN(C)C(=O)c1nc2n(n1)CCCN(c1nc(OC[C@@]34CCCN3C[C@H](F)C4)nc3cc(Br)c4nn(C)cc4c13)C2] | 1.29 min, 627.2/629.1 [M + H]⁺ (Method 5) |
| ((2R,7aS)-2- | I-126 | |
| fluorohexahydro-1H- | 8-[4-bromo-2-methyl-7- | |
| pyrrolizin-7a-yl)methanol | [[(2R,8S)-2-fluoro- | |
| | 1,2,3,5,6,7- | |
| | hexahydropyrrolizin-8- | |
| | yl]methoxy]pyrazolo[4,3- | |
| | f]quinazolin-9-yl]-N,N- | |
| | dimethyl-5,6,7,9-tetrahydro- | |
| | [1,2,4]triazolo[1,5- | |
| | a][1,4]diazepine-2- | |
| | carboxamide | |
| I-127 and 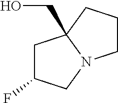 [SMILES: OC[C@@]12CCCN1C[C@H](F)C2] | Intermediate I-128 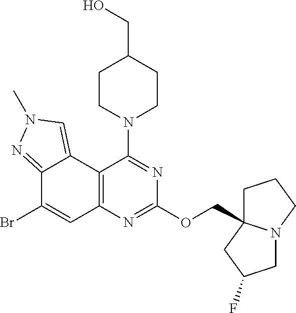 [SMILES: Cn1cc2c(n1)c(Br)cc1nc(OC[C@@]34CCCN3C[C@H](F)C4)nc(N3CCC(CO)CC3)c12] | 1.32 min, 533.1/535.0 [M + H]⁺ (Method 5) |
| ((2R,7aS)-2- | I-128 | |
| fluorohexahydro-1H- | [1-[4-bromo-7-[[(2R,8S)-2- | |
| pyrrolizin-7a-yl)methanol | fluoro-1,2,3,5,6,7- | |
| | hexahydropyrrolizin-8- | |
| | yl]methoxy]-2-methyl- | |
| | pyrazolo[4,3-f]quinazolin-9- | |
| | yl]-4-piperidyl]methanol | |
| I-129 and 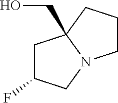 [SMILES: OC[C@@]12CCCN1C[C@H](F)C2] | Intermediate I-130 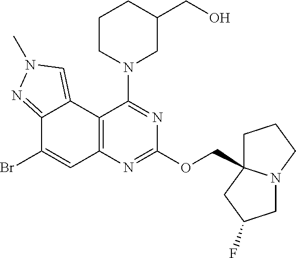 [SMILES: Cn1cc2c(n1)c(Br)cc1nc(OC[C@@]34CCCN3C[C@H](F)C4)nc(N3CCCC(CO)C3)c12] | 1.37 min, 533.1/535.0 [M + H]⁺ (Method 5) |
| ((2R,7aS)-2- | I-130 | |
| fluorohexahydro-1H- | [1-[4-bromo-7-[[(2R,8S)-2- | |
| pyrrolizin-7a-yl)methanol | fluoro-1,2,3,5,6,7- | |
| | hexahydropyrrolizin-8- | |
| | yl]methoxy]-2-methyl- | |
| | pyrazolo[4,3-f]quinazolin-9- | |
| | yl]-3-piperidyl]methanol | |
| I-131 and 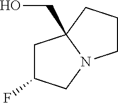 [SMILES: OC[C@@]12CCCN1C[C@H](F)C2] | Intermediate I-132 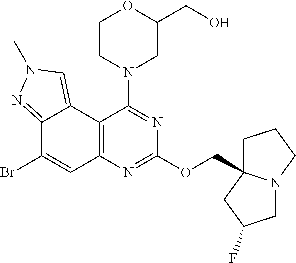 [SMILES: Cn1cc2c(n1)c(Br)cc1nc(OC[C@@]34CCCN3C[C@H](F)C4)nc(N3CCOC(CO)C3)c12] | 1.30 min, 535.0/537.0 [M + H]⁺ (Method 5) |
| ((2R,7aS)-2- | I-132 | |
| fluorohexahydro-1H- | [4-[4-bromo-7-[[(2R,8S)-2- | |
| pyrrolizin-7a-yl)methanol | fluoro-1,2,3,5,6,7- | |
| | hexahydropyrrolizin-8- | |
| | yl]methoxy]-2-methyl- | |
| | pyrazolo[4,3-f]quinazolin-9- | |
| | yl]morpholin-2-yl]methanol | |
| I-133 and 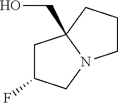 [SMILES: OC[C@@]12CCCN1C[C@H](F)C2] | Intermediate I-134 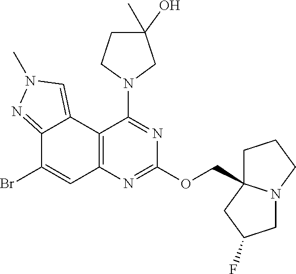 [SMILES: Cn1cc2c(n1)c(Br)cc1nc(OC[C@@]34CCCN3C[C@H](F)C4)nc(N3CCC(C)(O)C3)c12] | 1.19 min, 519.2/521.1 [M + H]⁺ (Method 2) |
| ((2R,7aS)-2- | I-134 | |
| fluorohexahydro-1H- | 1-[4-bromo-2-methyl-7- | |
| pyrrolizin-7a-yl)methanol | [[(2R,8S)-2-fluoro- | |
| | 1,2,3,5,6,7- | |
| | hexahydropyrrolizin-8- | |
| | yl]methoxy]pyrazolo[4,3- | |
| | f]quinazolin-9-yl]-3-methyl- | |
| | pyrrolidin-3-ol | |
| I-135 and 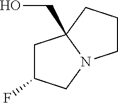 [SMILES: OC[C@@]12CCCN1C[C@H](F)C2] | Intermediate I-136 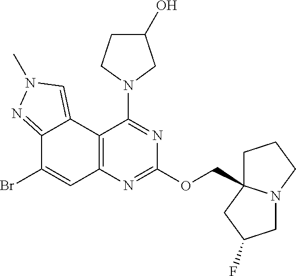 [SMILES: Cn1cc2c(n1)c(Br)cc1nc(OC[C@@]34CCCN3C[C@H](F)C4)nc(N3CCC(O)C3)c12] | 1.08 min, 505.1/507.1 [M + H]⁺ (Method 2) |
| ((2R,7aS)-2- | I-136 | |
| fluorohexahydro-1H- | 1-[4-bromo-2-methyl-7- | |
| pyrrolizin-7a-yl)methanol | [[(2R,8S)-2-fluoro- | |
| | 1,2,3,5,6,7- | |
| | hexahydropyrrolizin-8- | |
| | yl]methoxy]pyrazolo[4,3- | |
| | f]quinazolin-9-yl]pyrrolidin-3- | |
| | ol | |
| I-155 and 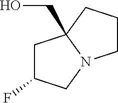 [SMILES: OC[C@@]12CCCN1C[C@H](F)C2] | Intermediate I-156 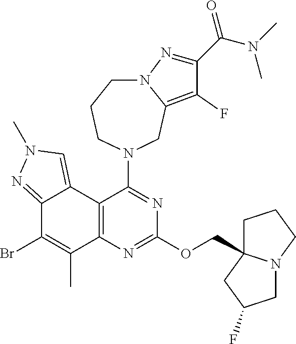 [SMILES: Cc1c(Br)c2nn(C)cc2c2c(N3CCCn4nc(C(=O)N(C)C)c(F)c4C3)nc(OC[C@@]34CCCN3C[C@H](F)C4)nc12] | 1.50 min, 658.2/660.0 [M + H]⁺ (Method 5) |
| ((2R,7aS)-2- | I-156 | |
| fluorohexahydro-1H- | 5-[4-bromo-7-[[(2R,8S)-2- | |
| pyrrolizin-7a-yl)methanol | fluoro-1,2,3,5,6,7- | |
| | hexahydropyrrolizin-8- | |
| | yl]methoxy]-2,5-dimethyl- | |
| | pyrazolo[4,3-f]quinazolin-9- | |
| | yl]-3-fluoro-N,N-dimethyl- | |
| | 4,6,7,8- | |
| | tetrahydropyrazolo[1,5- | |
| | a][1,4]diazepine-2- | |
| | carboxamide | |
| I-157 and 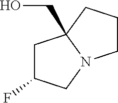 [SMILES: OC[C@@]12CCCN1C[C@H](F)C2] | Intermediate I-158 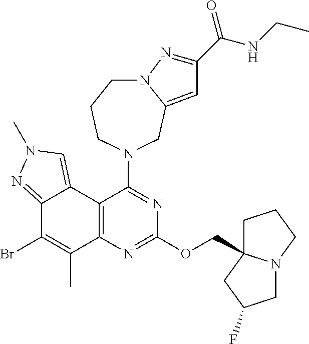 [SMILES: CCNC(=O)c1cc2n(n1)CCCN(c1nc(OC[C@@]34CCCN3C[C@H](F)C4)nc3c(C)c(Br)c4nn(C)cc4c13)C2] | 1.41 min, 540.2/642.2 [M + H]⁺ (Method 5) |
| ((2R,7aS)-2- | I-158 | |
| fluorohexahydro-1H- | 5-[4-bromo-2,5-dimethyl-7- | |
| pyrrolizin-7a-yl)methanol | [[(2R,8S)-2-fluoro- | |
| | 1,2,3,5,6,7- | |
| | hexahydropyrrolizin-8- | |
| | yl]methoxy]pyrazolo[4,3- | |
| | f]quinazolin-9-yl]-N-ethyl- | |
| | 4,6,7,8- | |
| | tetrahydropyrazolo[1,5- | |
| | a][1,4]diazepine-2- | |
| | carboxamide | |

### Intermediate I-140, 4-[4-[8-ethyl-7-fluoro-3-(methoxymethoxy)-1-naphthyl]-7-ethylsulfonyl-5-fluoro-2-methyl-pyrazolo[4,3-f]quinazolin-9-yl]-6-methyl-1,4-oxazepan-6-ol

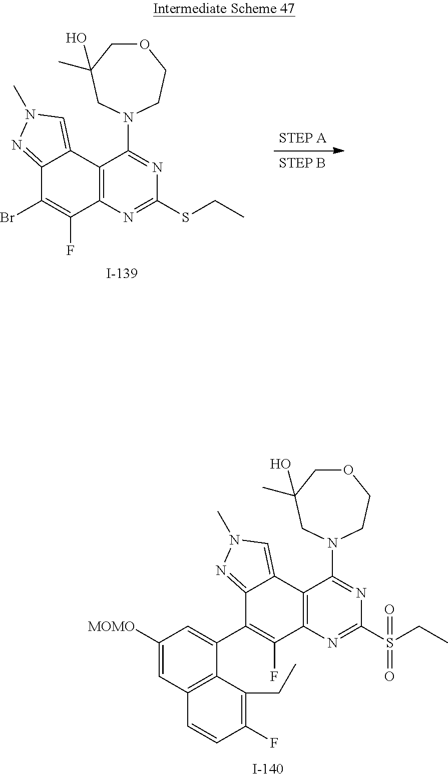

Step A, 4-[4-[8-ethyl-7-fluoro-3-(methoxymethoxy)-1-naphthyl]-7-ethylsulfanyl-5-fluoro-2-methyl-pyrazolo[4,3-f]quinazolin-9-yl]-6-methyl-1,4-oxazepan-6-ol. A solution of 4-(4-bromo-7-ethylsulfanyl-5-fluoro-2-methyl-pyrazolo[4,3-f]quinazolin-9-yl)-6-methyl-1,4-oxazepan-6-ol (520.9 mg, 1.11 mmol), 2-[8-ethyl-7-fluoro-3-(methoxymethoxy)-1-naphthyl]-4,4,5,5-tetramethyl-1,3,2-dioxaborolane (598.4 mg, 1.66 mmol) and Cesium Carbonate (721.66 mg, 2.21 mmol) in 1,4-Dioxane (5.5372 mL) and Water (1.8457 mL) was degassed with nitrogen for 2 min. cataCXium A Pd G3, Methanesulfonato (diadamantyl-n-butylphosphino)-2′amino-1,1′-biphenyl-2-yl)palladium(II) (80.65 mg, 0.11 mmol) was added. Reaction mixture allowed to stir at 100° C. for 90 min. Reaction mixture allowed to cool back down to room temperature. Partitioned between a layer of ethyl acetate (20 mL) and water (20 mL). Organic layer separated and aqueous layer extracted with ethyl acetate (20 mL). Organic layers combined, washed with a saturated solution of brine (10 mL), dried over sodium sulfate, filtered and concentrated under reduced pressure. Purification by flash column chromatography (eluting in 20-100% EtOAc in Pet. Ether) afforded 4-[4-[8-ethyl-7-fluoro-3-(methoxymethoxy)-1-naphthyl]-7-ethylsulfanyl-5-fluoro-2-methyl-pyrazolo[4,3-f]quinazolin-9-yl]-6-methyl-1,4-oxazepan-6-ol (457.4 mg, 0.7333 mmol, 66% yield) as a brown solid.

UPLC-MS (ES⁺, Method 2): 2.05 min, m/z 624.7 [M+H]⁺.

Step B, 4-[4-[8-ethyl-7-fluoro-3-(methoxymethoxy)-1-naphthyl]-7-ethylsulfonyl-5-fluoro-2-methyl-pyrazolo[4,3-f]quinazolin-9-yl]-6-methyl-1,4-oxazepan-6-ol. At 0° C., m-chloroperbenzoic acid, MCPBA (71.86 mg, 0.32 mmol) was added to a solution of 4-[4-[8-ethyl-7-fluoro-3-(methoxymethoxy)-1-naphthyl]-7-ethylsulfanyl-5-fluoro-2-methyl-pyrazolo[4,3-f]quinazolin-9-yl]-6-methyl-1,4-oxazepan-6-ol (80. mg, 0.13 mmol) in DCM (1.2826 mL). Afterwards, allowed to stir at room temperature for 2 h. Additional m-chloroperbenzoic acid, MCPBA (11.07 mg, 0.06 mmol) was added. Reaction mixture allowed to stir at room temperature for 15 min. Reaction mixture quenched with a saturated solution of sodium thiosulfate. Partitioned between a layer of ethyl acetate (20 mL) and water (20 mL). Organic layer separated, aqueous layer extracted with ethyl acetate (20 mL) and organic layers combined. Combined organic layers washed with a saturated solution of sodium bicarbonate (2×10 mL), a saturated solution of brine (10 mL), dried over sodium sulfate, filtered and concentrated under reduced pressure to afford 4-[4-[8-ethyl-7-fluoro-3-(methoxymethoxy)-1-naphthyl]-7-ethylsulfonyl-5-fluoro-2-methyl-pyrazolo[4,3-f]quinazolin-9-yl]-6-methyl-1,4-oxazepan-6-ol (84.104 mg, 0.1283 mmol, 100% yield) as an off-white solid.

UPLC-MS (ES⁺, Method 2): 1.79 min, m/z 656.4 [M+H] *.

The following examples in table 16 were made by analogy with I-140 (Intermediate scheme 47) starting from the intermediate and building blocks for step A as described in the table. Where the intermediate for step A is already the sulfone (e.g. I-97), step B was not required. The catalyst cataCXium A Pd G3 could be substituted for Pd(dtbpf)Cl₂ in step A.

TABLE 16

| Intermediate Building block | Intermediate Structure/Name | UPLC-MS (ES+): min, m/z |
|---|---|---|
| Intermediate I-141 and 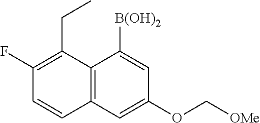 | Intermediate I-142 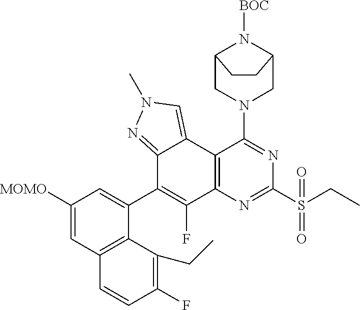 | 2.07 min, 737.6 [M + H]⁺ (Method 2) |
| [8-ethyl-7-fluoro-3- | I-142 | |
| (methoxymethoxy)naphthalen- | tert-butyl 3-[4-[8-ethyl-7-fluoro-3- | |
| 1-yl]boronic acid | (methoxymethoxy)-1-naphthyl]-7- | |
| | ethylsulfonyl-5-fluoro-2-methyl- | |
| | pyrazolo[4,3-f]quinazolin-9-yl]-3,8- | |
| | diazabicyclo[3.2.1]octane-8-carboxylate | |
| Intermediate I-144 and 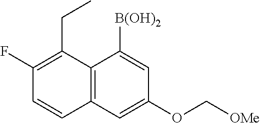 | Intermediate I-145 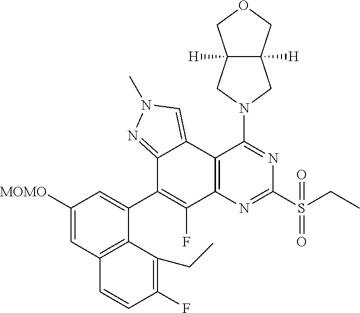 | 1.86 min, 638.4 [M + H]⁺ (Method 2) |
| [8-ethyl-7-fluoro-3- | I-145 | |
| (methoxymethoxy)naphthalen- | (3aS,6aR)-5-[4-[8-ethyl-7-fluoro-3- | |
| 1-yl]boronic acid | (methoxymethoxy)-1-naphthyl]-7- | |
| | ethylsulfonyl-5-fluoro-2-methyl- | |
| | pyrazolo[4,3-f]quinazolin-9-yl]- | |
| | 1,3,3a,4,6,6a-hexahydrofuro[3,4-c]pyrrole | |
| Intermediate I-144 and 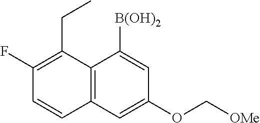 | Intermediate I-146 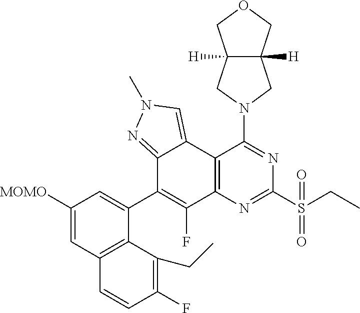 | 1.82 min, 638.4 [M + H]⁺ (Method 2) |
| [8-ethyl-7-fluoro-3- | I-146 | |
| (methoxymethoxy)naphthalen- | (3aS,6aS)-5-[4-[8-ethyl-7-fluoro-3- | |
| 1-yl]boronic acid | (methoxymethoxy)-1-naphthyl]-7- | |
| | ethylsulfonyl-5-fluoro-2-methyl- | |
| | pyrazolo[4,3-f]quinazolin-9-yl]- | |
| | 1,3,3a,4,6,6a-hexahydrofuro[3,4-c]pyrrole | |
| Intermediate I-147 and 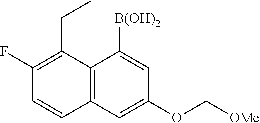 | Intermediate I-148 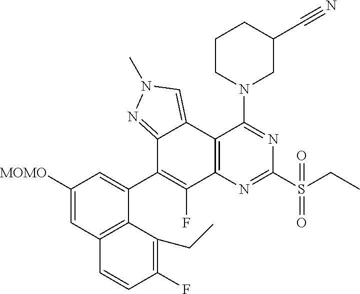 | 1.88 min, 635.4 [M + H]⁺ (Method 2) |
| [8-ethyl-7-fluoro-3- | I-148 | |
| (methoxymethoxy)naphthalen- | 1-[4-[8-ethyl-7-fluoro-3- | |
| 1-yl]boronic acid | (methoxymethoxy)-1-naphthyl]-7- | |
| | ethylsulfonyl-5-fluoro-2-methyl- | |
| | pyrazolo[4,3-f]quinazolin-9-yl]piperidine- | |
| | 3-carbonitrile | |
| Intermediate I-149 and 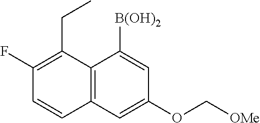 | Intermediate I-150 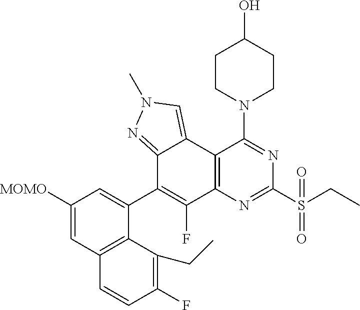 | 1.77 min, 626.4 [M + H]⁺ (Method 2) |
| [8-ethyl-7-fluoro-3- | I-150 | |
| (methoxymethoxy)naphthalen- | 1-[4-[8-ethyl-7-fluoro-3- | |
| 1-yl]boronic acid | (methoxymethoxy)-1-naphthyl]-7- | |
| | ethylsulfonyl-5-fluoro-2-methyl- | |
| | pyrazolo[4,3-f]quinazolin-9-yl]piperidin-4- | |
| | ol | |
| Intermediate I-151 and 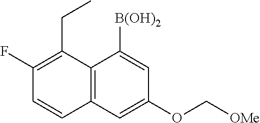 | Intermediate I-152 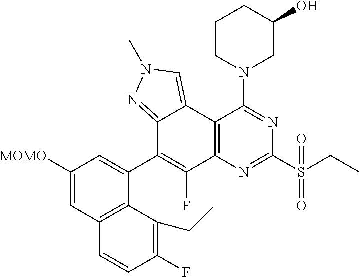 | 1.80 min, 626.4 [M + H]⁺ (Method 2) |
| [8-ethyl-7-fluoro-3- | I-152 | |
| (methoxymethoxy)naphthalen- | (3R)-1-[4-[8-ethyl-7-fluoro-3- | |
| 1-yl]boronic acid | (methoxymethoxy)-1-naphthyl]-7- | |
| | ethylsulfonyl-5-fluoro-2-methyl- | |
| | pyrazolo[4,3-f]quinazolin-9-yl]piperidin-3- | |
| | ol | |
| Intermediate I-153 and 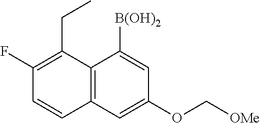 | Intermediate I-154 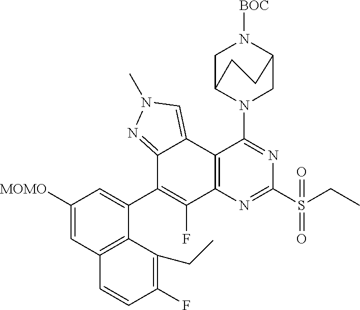 | 2.19 min, 737.3 [M + H]⁺ (Method 5) |
| [8-ethyl-7-fluoro-3- | I-154 | |
| (methoxymethoxy)naphthalen- | tert-butyl 5-[4-[8-ethyl-7-fluoro-3- | |
| 1-yl]boronic acid | (methoxymethoxy)-1-naphthyl]-7- | |
| | ethylsulfonyl-5-fluoro-2-methyl- | |
| | pyrazolo[4,3-f]quinazolin-9-yl]-2,5- | |
| | diazabicyclo[2.2.2]octane-2-carboxylate | |
| Intermediate I-159 and 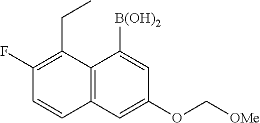 | Intermediate I-160 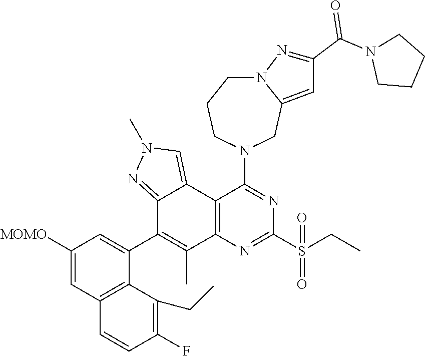 | 2.04 min, 755.3 [M + H]⁺ (Method 5) |
| [8-ethyl-7-fluoro-3- | I-160 | |
| (methoxymethoxy)naphthalen- | [5-[4-[8-ethyl-7-fluoro-3- | |
| 1-yl]boronic acid | (methoxymethoxy)-1-naphthyl]-7- | |
| | ethylsulfonyl-2,5-dimethyl-pyrazolo[4,3- | |
| | f]quinazolin-9-yl]-4,6,7,8- | |
| | tetrahydropyrazolo[1,5-a][1,4]diazepin-2- | |
| | yl]-pyrrolidin-1-yl-methanone | |
| Intermediate I-161 and 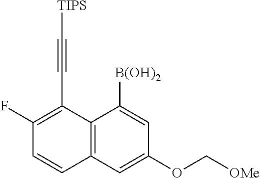 | Intermediate I-162 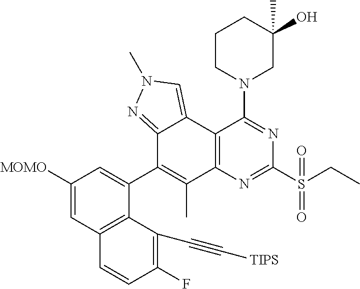 | 2.37 min, 789.4 [M + H]⁺ (Method 2) |
| [7-fluoro-3-(methoxymethoxy)- | I-162 | |
| 8-(2-triisopropylsilylethynyl)-1- | (3R)-1-[7-ethylsulfonyl-4-[7-fluoro-3- | |
| naphthyl]boronic acid | (methoxymethoxy)-8-(2- | |
| | triisopropylsilylethynyl)-1-naphthyl]-2,5- | |
| | dimethyl-pyrazolo[4,3-f]quinazolin-9-yl]-3- | |
| | methyl-piperidin-3-ol | |
| Intermediate I-166 and 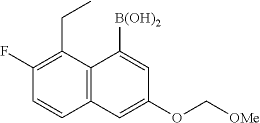 | Intermediate I-167 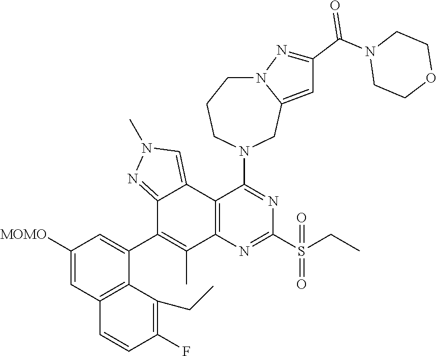 | 1.89 min, 775.3 [M + H]⁺ (Method 5) |
| [8-ethyl-7-fluoro-3- | I-167 | |
| (methoxymethoxy)naphthalen- | [5-[4-[8-ethyl-7-fluoro-3- | |
| 1-yl]boronic acid | (methoxymethoxy)-1-naphthyl]-7- | |
| | ethylsulfonyl-5-fluoro-2-methyl- | |
| | pyrazolo[4,3-f]quinazolin-9-yl]-4,6,7,8- | |
| | tetrahydropyrazolo[1,5-a][1,4]diazepin-2- | |
| | yl]-morpholino-methanone | |
| Intermediate I-168 and 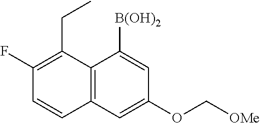 | Intermediate I-169 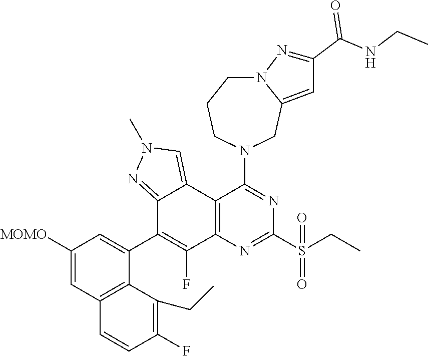 | 1.92 min, 733.3 [M + H]⁺ (Method 5) |
| [8-ethyl-7-fluoro-3- | I-169 | |
| (methoxymethoxy)naphthalen- | N-ethyl-5-[4-[8-ethyl-7-fluoro-3- | |
| 1-yl]boronic acid | (methoxymethoxy)-1-naphthyl]-7- | |
| | ethylsulfonyl-5-fluoro-2-methyl- | |
| | pyrazolo[4,3-f]quinazolin-9-yl]-4,6,7,8- | |
| | tetrahydropyrazolo[1,5-a][1,4]diazepine- | |
| | 2-carboxamide | |
| Intermediate I-170 and 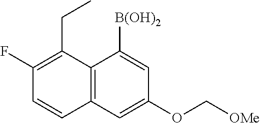 | Intermediate I-171 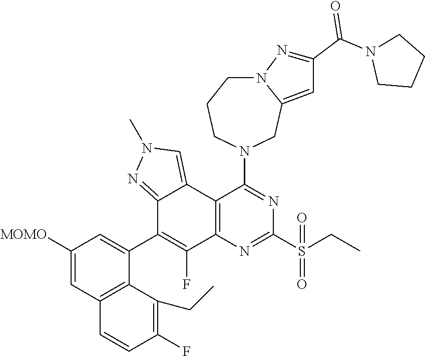 | 1.95 min, 759.3 [M + H]⁺ (Method 5) |
| [8-ethyl-7-fluoro-3- | I-171 | |
| (methoxymethoxy)naphthalen- | [5-[4-[8-ethyl-7-fluoro-3- | |
| 1-yl]boronic acid | (methoxymethoxy)-1-naphthyl]-7- | |
| | ethylsulfonyl-5-fluoro-2-methyl- | |
| | pyrazolo[4,3-f]quinazolin-9-yl]-4,6,7,8- | |
| | tetrahydropyrazolo[1,5-a][1,4]diazepin-2- | |
| | yl]-pyrrolidin-1-yl-methanone | |
| Intermediate I-172 and 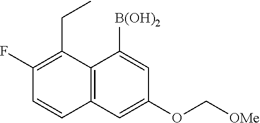 | Intermediate I-173 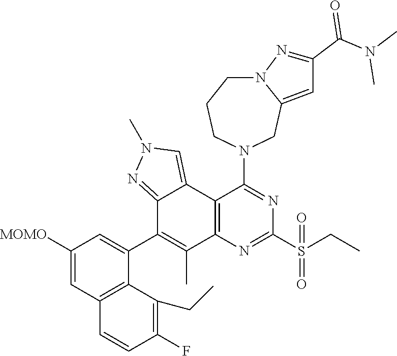 | 1.91 min, 730.3 [M + H]⁺ (Method 5) |
| [8-ethyl-7-fluoro-3- | I-173 | |
| (methoxymethoxy)naphthalen- | 8-[4-[8-ethyl-7-fluoro-3- | |
| 1-yl]boronic acid | (methoxymethoxy)-1-naphthyl]-7- | |
| | ethylsulfonyl-2,5-dimethyl-pyrazolo[4,3- | |
| | f]quinazolin-9-yl]-N,N-dimethyl-5,6,7,9- | |
| | tetrahydro-[1,2,4]triazolo[1,5- | |
| | a][1,4]diazepine-2-carboxamide | |
| Intermediate I-174 and 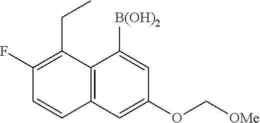 | Intermediate I-175 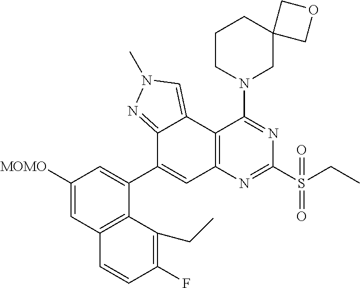 | 1.88 min, 634.4 [M + H]⁺ (Method 2) |
| [8-ethyl-7-fluoro-3- | I-175 | |
| (methoxymethoxy)naphthalen- | 8-[4-[8-ethyl-7-fluoro-3- | |
| 1-yl]boronic acid | (methoxymethoxy)-1-naphthyl]-7- | |
| | ethylsulfonyl-2-methyl-pyrazolo[4,3- | |
| | f]quinazolin-9-yl]-2-oxa-8- | |
| | azaspiro[3.5]nonane | |
| Intermediate I-176 and 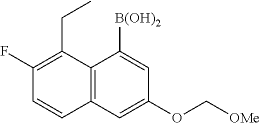 | Intermediate I-177 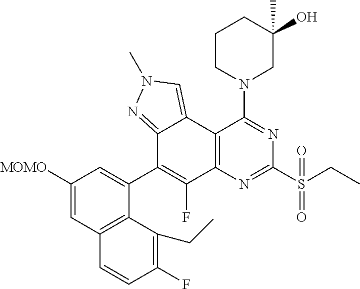 | 1.99 min, 640.2 [M + H]⁺ (Method 5) |
| [8-ethyl-7-fluoro-3- | I-177 | |
| (methoxymethoxy)naphthalen- | (3R)-1-[4-[8-ethyl-7-fluoro-3- | |
| 1-yl]boronic acid | (methoxymethoxy)-1-naphthyl]-7- | |
| | ethylsulfonyl-5-fluoro-2-methyl- | |
| | pyrazolo[4,3-f]quinazolin-9-yl]-3-methyl- | |
| | piperidin-3-ol | |
| Intermediate I-161 and 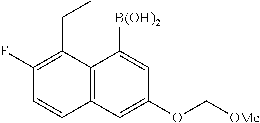 | Intermediate I-178 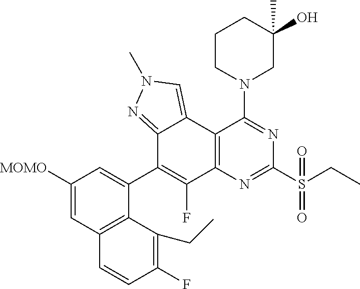 | 2.07 min, 636.2 [M + H]⁺ (Method 5) |
| [8-ethyl-7-fluoro-3- | I-178 | |
| (methoxymethoxy)naphthalen- | (3R)-1-[4-[8-ethyl-7-fluoro-3- | |
| 1-yl]boronic acid | (methoxymethoxy)-1-naphthyl]-7- | |
| | ethylsulfonyl-2,5-dimethyl-pyrazolo[4,3- | |
| | f]quinazolin-9-yl]-3-methyl-piperidin-3-ol | |
| Intermediate I-179 and 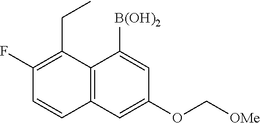 | Intermediate I-180 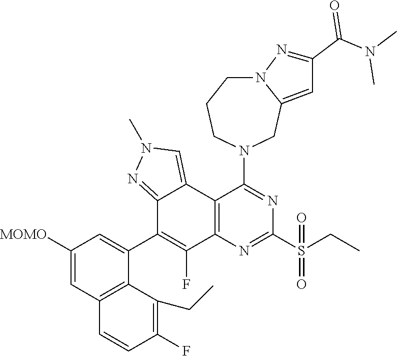 | 1.76 min, 733.6 [M + H]⁺ (Method 2) |
| [8-ethyl-7-fluoro-3- | I-180 | |
| (methoxymethoxy)naphthalen- | 5-[4-[8-ethyl-7-fluoro-3- | |
| 1-yl]boronic acid | (methoxymethoxy)-1-naphthyl]-7- | |
| | ethylsulfonyl-5-fluoro-2-methyl- | |
| | pyrazolo[4,3-f]quinazolin-9-yl]-N,N- | |
| | dimethyl-4,6,7,8-tetrahydropyrazolo[1,5- | |
| | a][1,4]diazepine-2-carboxamide | |
| Intermediate I-181 and 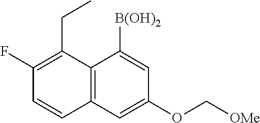 | Intermediate I-182 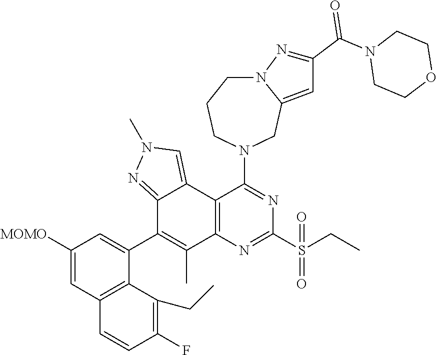 | 1.96 min, 771.3 [M + H]⁺ (Method 5) |
| [8-ethyl-7-fluoro-3- | I-182 | |
| (methoxymethoxy)naphthalen- | [5-[4-[8-ethyl-7-fluoro-3- | |
| 1-yl]boronic acid | (methoxymethoxy)-1-naphthyl]-7- | |
| | ethylsulfonyl-2,5-dimethyl-pyrazolo[4,3- | |
| | f]quinazolin-9-yl]-4,6,7,8- | |
| | tetrahydropyrazolo[1,5-a][1,4]diazepin-2- | |
| | yl]-morpholino-methanone | |
| Intermediate I-183 and 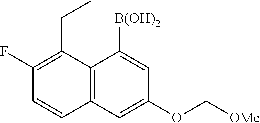 | Intermediate I-184 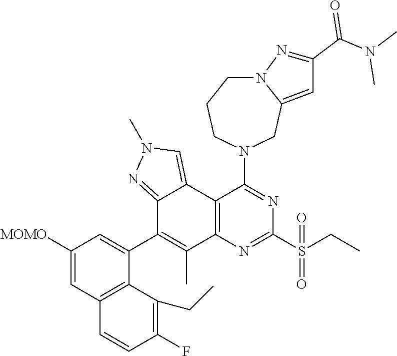 | 1.83 min, 729.6 [M + H]⁺ (Method 2) |
| [8-ethyl-7-fluoro-3- | I-184 | |
| (methoxymethoxy)naphthalen- | 5-[4-[8-ethyl-7-fluoro-3- | |
| 1-yl]boronic acid | (methoxymethoxy)-1-naphthyl]-7- | |
| | ethylsulfonyl-2,5-dimethyl-pyrazolo[4,3- | |
| | f]quinazolin-9-yl]-N,N-dimethyl-4,6,7,8- | |
| | tetrahydropyrazolo[1,5-a][1,4]diazepine- | |
| | 2-carboxamide | |
| Intermediate I-185 and 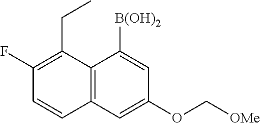 | Intermediate I-186 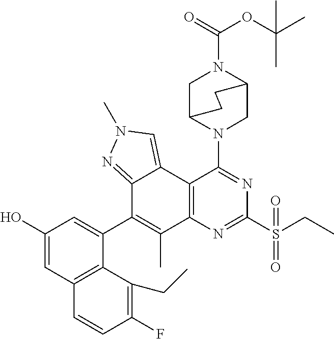 | 1.83 min, 733.3 [M + H]⁺ (Method 5) |
| [8-ethyl-7-fluoro-3- | I-186 | |
| (methoxymethoxy)naphthalen- | tert-butyl 5-[4-[8-ethyl-7-fluoro-3- | |
| 1-yl]boronic acid | (methoxymethoxy)-1-naphthyl]-7- | |
| | ethylsulfonyl-2,5-dimethyl-pyrazolo[4,3- | |
| | f]quinazolin-9-yl]-2,5- | |
| | diazabicyclo[2.2.2]octane-2-carboxylate | |
| Intermediate I-187 and 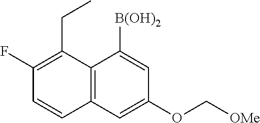 | Intermediate I-188 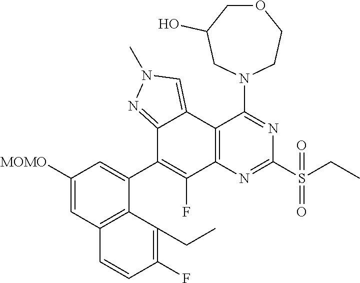 | 1.89 min, 642.2 [M + H]⁺ (Method 5) |
| [8-ethyl-7-fluoro-3- | I-188 | |
| (methoxymethoxy)naphthalen- | 4-[4-[8-ethyl-7-fluoro-3- | |
| 1-yl]boronic acid | (methoxymethoxy)-1-naphthyl]-7- | |
| | ethylsulfonyl-5-fluoro-2-methyl- | |
| | pyrazolo[4,3-f]quinazolin-9-yl]-1,4- | |
| | oxazepan-6-ol | |
| Intermediate I-189 and 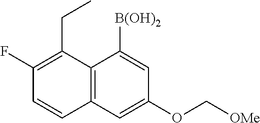 | Intermediate I-190 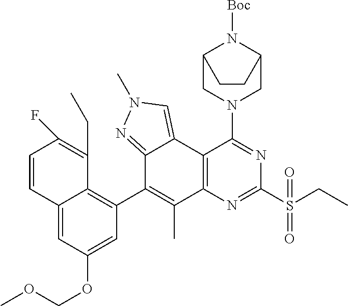 | 2.06 min, 737.2 [M + H]⁺ (Method 2) |
| [8-ethyl-7-fluoro-3- | I-190 | |
| (methoxymethoxy)naphthalen- | tert-butyl 3-[4-[8-ethyl-7-fluoro-3- | |
| 1-yl]boronic acid | (methoxymethoxy)-1-naphthyl]-7- | |
| | ethylsulfonyl-5-fluoro-2-methyl- | |
| | pyrazolo[4,3-f]quinazolin-9-yl]-3,8- | |
| | diazabicyclo[3.2.1]octane-8-carboxylate | |
| Intermediate I-191 and 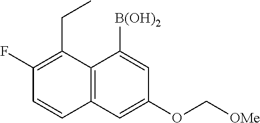 | Intermediate I-192 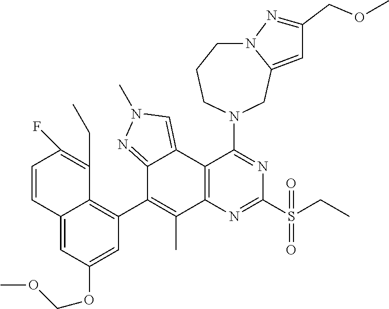 | 2.02 min, 702.3 [M + H]⁺ (Method 5) |
| [8-ethyl-7-fluoro-3- | I-192 | |
| (methoxymethoxy)naphthalen- | 4-[8-ethyl-7-fluoro-3-(methoxymethoxy)- | |
| 1-yl]boronic acid | 1-naphthyl]-7-ethylsulfonyl-9-[2- | |
| | (methoxymethyl)-4,6,7,8- | |
| | tetrahydropyrazolo[1,5-a][1,4]diazepin-5- | |
| | yl]-2,5-dimethyl-pyrazolo[4,3- | |
| | f]quinazoline | |
| Intermediate I-193 and 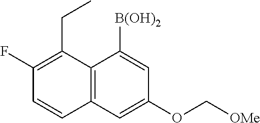 | Intermediate I-194 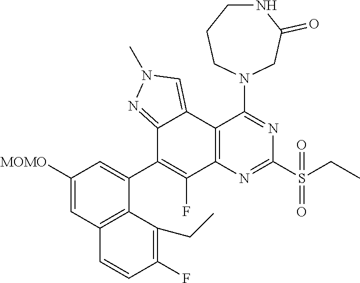 | 1.67 min, 639.4 [M + H]⁺ (Method 5) |
| [8-ethyl-7-fluoro-3- | 4-[4-[8-ethyl-7-fluoro-3- | |
| (methoxymethoxy)naphthalen- | (methoxymethoxy)-1-naphthyl]-7- | |
| 1-yl]boronic acid | ethylsulfonyl-5-fluoro-2-methyl- | |
| | pyrazolo[4,3-f]quinazolin-9-yl]-1,4- | |
| | diazepan-2-one | |
| Intermediate I-118 and 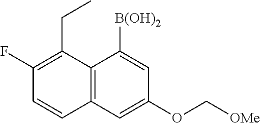 | Intermediate I-195 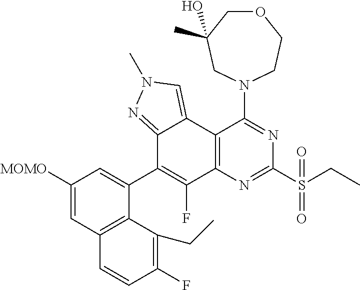 | 1.93 min, 656.2 [M + H]⁺ (Method 5) |
| [8-ethyl-7-fluoro-3- | I-195 | |
| (methoxymethoxy)naphthalen- | (6S)-4-[4-[8-ethyl-7-fluoro-3- | |
| 1-yl]boronic acid | (methoxymethoxy)-1-naphthyl]-7- | |
| | ethylsulfonyl-5-fluoro-2-methyl- | |
| | pyrazolo[4,3-f]quinazolin-9-yl]-6-methyl- | |
| | 1,4-oxazepan-6-ol | |

### Intermediate I-143, 1-[[4-[8-ethyl-7-fluoro-3-(methoxymethoxy)-1-naphthyl]-5-fluoro-9-(6-hydroxy-6-methyl-1,4-oxazepan-4-yl)-2-methyl-pyrazolo[4,3-f]quinazolin-7-yl]oxymethyl]cyclopropanecarbaldehyde

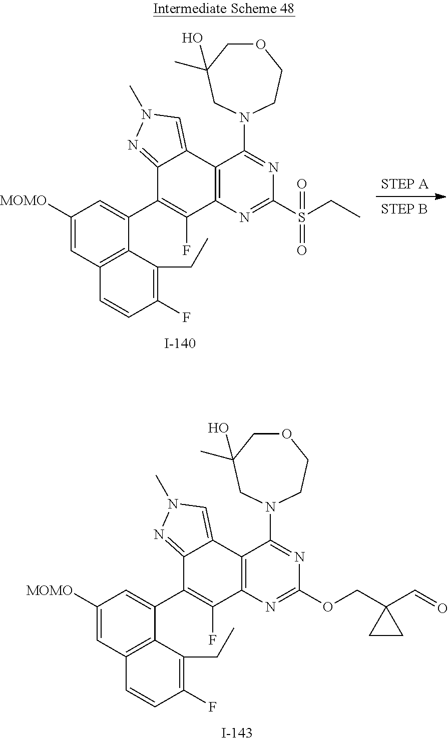

Step A, 4-[4-[8-ethyl-7-fluoro-3-(methoxymethoxy)-1-naphthyl]-5-fluoro-7-[[1-(hydroxymethyl)cyclopropyl]methoxy]-2-methyl-pyrazolo[4,3-f]quinazolin-9-yl]-6-methyl-1,4-oxazepan-6-ol. To a solution of [1-(Hydroxymethyl)cyclopropyl]methanol (54.51 mg, 0.53 mmol) in THF (2 mL) under N₂ was added dropwise 1M Lithium bis(trimethylsilyl)amide in THF (0.53 mL, 0.53 mmol) at 25° C. The reaction mixture was stirred for 20 mins then 4-[4-[8-ethyl-7-fluoro-3-(methoxymethoxy)-1-naphthyl]-7-ethylsulfonyl-5-fluoro-2-methyl-pyrazolo[4,3-f]quinazolin-9-yl]-6-methyl-1,4-oxazepan-6-ol (70. mg, 0.11 mmol) in THF (2 mL) was added in one portion. The reaction mixture was left to stir at room temperature for 2 h. The reaction mixture was quenched with water and NaHCO₃ (sat. aq), extracted with EtOAc (×3) and washed with brine, dried over magnesium sulfate, filtered and evaporated to afford 4-[4-[8-ethyl-7-fluoro-3-(methoxymethoxy)-1-naphthyl]-5-fluoro-7-[1-(hydroxymethyl)cyclopropyl]methoxy]-2-methyl-pyrazolo[4,3-f]quinazolin-9-yl]-6-methyl-1,4-oxazepan-6-ol (70.85 mg, 0.1067 mmol, 100% yield) as a yellow oil.

UPLC-MS (ES⁺, Method 2): 1.77 min, m/z 664.8 [M+H]⁺.

Step B, 1-[4-[8-ethyl-7-fluoro-3-(methoxymethoxy)-1-naphthyl]-5-fluoro-9-(6-hydroxy-6-methyl-1,4-oxazepan-4-yl)-2-methyl-pyrazolo[4,3-f]quinazolin-7-ylloxymethyl]cyclopropanecarbaldehyde. To a solution of 4-[4-[8-ethyl-7-fluoro-3-(methoxymethoxy)-1-naphthyl]-5-fluoro-7-[1-(hydroxymethyl)cyclopropyl]methoxy]-2-methyl-pyrazolo[4,3-f]quinazolin-9-yl]-6-methyl-1,4-oxazepan-6-ol (70.85 mg, 0.11 mmol) in DCM (3 mL) was added Dess Martin periodinane (54.33 mg, 0.13 mmol). The reaction mixture was stirred at room temperature for 1 hour. Additional Dess Martin periodinane (90.55 mg, 0.21 mmol) was added and the mixture left to stir overnight. Additional Dess Martin periodinane (90.55 mg, 0.21 mmol) was added and the reaction left to stir for a further 48 hours. The reaction mixture was quenched with Na₂S₂O₄ (sat. aq):NaHCO₃ (sat. aq) (1:1), extracted with DCM (×3) and washed with sodium bicarbonate aq and brine, dried over magnesium sulfate, filtered and concentrated in vacuo to afford 1-[4-[8-ethyl-7-fluoro-3-(methoxymethoxy)-1-naphthyl]-5-fluoro-9-(6-hydroxy-6-methyl-1,4-oxazepan-4-yl)-2-methyl-pyrazolo[4,3-f]quinazolin-7-yl]oxymethyl]cyclopropanecarbaldehyde (90 mg, 0.1115 mmol, 104% yield) as a yellow oil.

UPLC-MS (ES⁺, Method 2): 2.00 min, m/z 662.3 [M+H]⁺.

### Intermediate I-111, 4-[8-ethyl-7-fluoro-3-(methoxymethoxy)-1-naphthyl]-7-ethylsulfonyl-2,5-dimethyl-pyrazolo[4,3-f]quinazolin-9-ol

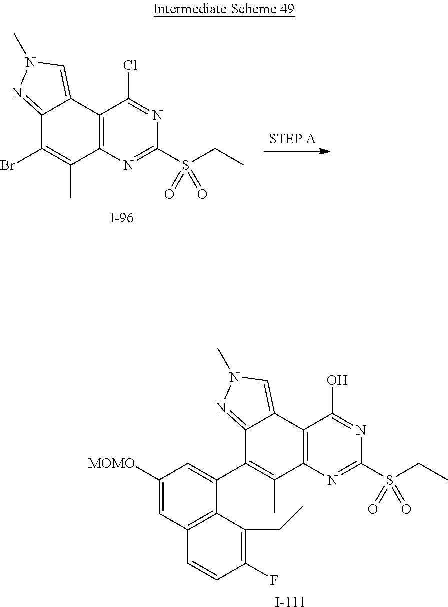

Step A, 4-[8-ethyl-7-fluoro-3-(methoxymethoxy)-1-naphthyl]-7-ethylsulfonyl-2,5-dimethyl-pyrazolo[4,3-f]quinazolin-9-ol. A solution of 2-[8-ethyl-7-fluoro-3-(methoxymethoxy)-1-naphthyl]-4,4,5,5-tetramethyl-1,3,2-dioxaborolane (358.71 mg, 1. mmol), 4-bromo-7-[[(2R,8S)-2-fluoro-1,2,3,5,6,7-hexahydropyrrolizin-8-yl]methoxy]-9-[3-(3-methoxypropyl)-3,8-diazabicyclo[3.2.1]octan-8-yl]-2-methyl-pyrazolo[4,3-f]quinazoline (500. mg, 0.83 mmol) and cesium carbonate (540.74 mg, 1.66 mmol) in 1,4-Dioxane (6.9878 mL) and Water (1.3102 mL) was degassed with nitrogen and [1,1′-Bis(di-tert-butylphosphino)ferrocene]dichloropalladium(II) (54.08 mg, 0.08 mmol) added. The reaction mixture was heated to 100° C. for 30 minutes. The reaction mixture was cooled to room temperature then filtered through a phase separator cartridge, washing with MeOH. The filtrate was concentrated under reduced to afford the desired product 4-[8-ethyl-7-fluoro-3-(methoxymethoxy)-1-naphthyl]-7-ethylsulfonyl-2,5-dimethyl-pyrazolo[4,3-f]quinazolin-9-ol (134 mg, 0.25 mmol, 100% yield) as an oil.

UPLC-MS (ES⁺, Method 5): 2.01 min, m/z 539.1 [M+H]⁺.

### Intermediate I-112, 4-[4-[8-ethyl-7-fluoro-3-(methoxymethoxy)-1-naphthyl]-7-ethylsulfonyl-2,5-dimethyl-pyrazolo[4,3-f]quinazolin-9-yl]-6-methyl-1,4-oxazepan-6-ol

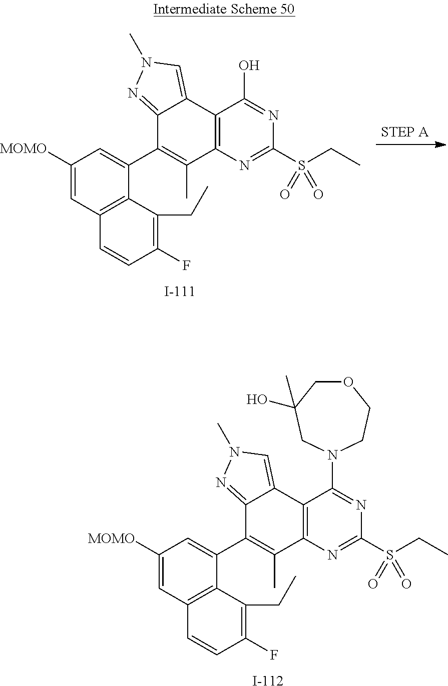

Step A, 4-[4-[8-ethyl-7-fluoro-3-(methoxymethoxy)-1-naphthyl]-7-ethylsulfonyl-2,5-dimethyl-pyrazolo[4,3-f]quinazolin-9-yl]-6-methyl-1,4-oxazepan-6-ol. To a stirring solution of 4-[8-ethyl-7-fluoro-3-(methoxymethoxy)-1-naphthyl]-7-ethylsulfonyl-2,5-dimethyl-pyrazolo[4,3-f]quinazolin-9-ol (110 mg, 0.2 mmol), 6-Methyl-1,4-oxazepan-6-ol.HCl (37.66 mg, 0.22 mmol) and N,N-Diisopropylethylamine (0.21 mL, 1.23 mmol) in THF (2 mL) was added HATU (93.19 mg, 0.25 mmol). The reaction mixture was heated to 71° C. and left to stir overnight. The reaction mixture was cooled to room temperature, diluted with brine, extracted with ethyl acetate and washed with water. The organics were dried over magnesium sulfate, filtered, and evaporated to give 4-[4-[8-ethyl-7-fluoro-3-(methoxymethoxy)-1-naphthyl]-7-ethylsulfonyl-2,5-dimethyl-pyrazolo[4,3-f]quinazolin-9-yl]-6-methyl-1,4-oxazepan-6-ol (120 mg, 0.1841 mmol, 90% yield) as a brown powder.

UPLC-MS (ES⁺, Method 5): 2.04 min, m/z 652.3 [M+H]⁺.

### EXAMPLES

### Example 1, 5-ethyl-6-fluoro-4-[2-methyl-9-(1,4-oxazepan-4-yl)-7-[[(2R,8S)-2-fluoro-1,2,3,5,6,7-hexahydropyrrolizin-8-yl]methoxy]pyrazolo[4,3-f]quinazolin-4-yl]naphthalen-2-ol

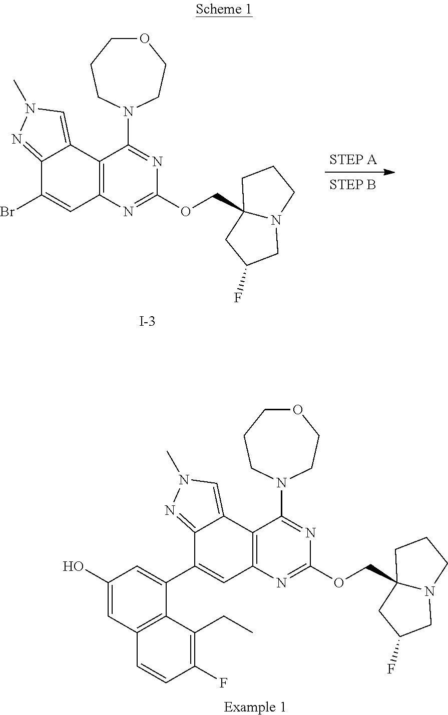

Example 1 was prepared following the route in scheme 1.

Step A, 4-[4-[8-ethyl-7-fluoro-3-(methoxymethoxy)-1-naphthyl]-2-methyl-7-[[(2R,8S)-2-fluoro-1,2,3,5,6,7-hexahydropyrrolizin-8-yl]methoxy]pyrazolo[4,3-f]quinazolin-9-yl]-1,4-oxazepane. 1,1′-Bis(di-tert-butylphosphino)ferrocene]dichloropalladium(II) (5.02 mg, 0.01 mmol) was added to a solution of 4-[4-bromo-2-methyl-7-[(2R,8S)-2-fluoro-1,2,3,5,6,7-hexahydropyrrolizin-8-yl]methoxy]pyrazolo[4,3-f]quinazolin-9-yl]-1,4-oxazepane (1-3) (40 mg, 0.08 mmol), [8-ethyl-7-fluoro-3-(methoxymethoxy) naphthalen-1-yl]boronic acid (27.84 mg, 0.1 mmol) and cesium carbonate (50.2 mg, 0.15 mmol) in 1,4-Dioxane (0.5 mL) and water (0.1 mL), which had been degassed for 10 minutes with N₂. The mixture was heated to 100° C. overnight. The solvent was removed in vacuo, and loaded onto Isolute HM-N with DCM. The crude was purified by flash column chromatography (5 g KP-amino silica, 5-80% EtOAc in petroleum ether). The desired fractions were concentrated to afford 4-[4-[8-ethyl-7-fluoro-3-(methoxymethoxy)-1-naphthyl]-2-methyl-7-[(2R,8S)-2-fluoro-1,2,3,5,6,7-hexahydropyrrolizin-8-yl]methoxy]pyrazolo[4,3-f]quinazolin-9-yl]-1,4-oxazepane (14 mg, 0.021 mmol, 27% yield) as a colourless glassy solid.

UPLC-MS (ES⁺, Method 1): 1.94 min, m/z 673.5 [M+H]⁺.

Step B, 5-ethyl-6-fluoro-4-[2-methyl-9-(1,4-oxazepan-4-yl)-7-[(2R,8S)-2-fluoro-1,2,3,5,6,7-hexahydropyrrolizin-8-yl]methoxy]pyrazolo[4,3-f]quinazolin-4-yl]naphthalen-2-ol. Trifluoroacetic acid (0.02 mL, 0.31 mmol) was added to a solution of 4-[4-[8-ethyl-7-fluoro-3-(methoxymethoxy)-1-naphthyl]-2-methyl-7-[(2R,8S)-2-fluoro-1,2,3,5,6,7-hexahydropyrrolizin-8-yl]methoxy]pyrazolo[4,3-f]quinazolin-9-yl]-1,4-oxazepane (14 mg, 0.02 mmol) and triethylsilane (0.02 mL, 0.1 mmol) in DCM (1 mL), and stirred at room temperature for 2 hours. The solvent was removed in vacuo and residue redissolved in MeOH. The solution was passed through an SCX-2 column, washed with MeOH, and eluted with 1N NH₃ in MeOH. The eluent was concentrated to afford a yellow glassy solid. The crude was purified by flash column chromatography (5 g KP-amino silica, 50-100% EtOAc in petroleum ether, followed by 0-5% MeOH in DCM). The desired fractions were concentrated to afford 5-ethyl-6-fluoro-4-[2-methyl-9-(1,4-oxazepan-4-yl)-7-[[(2R,8S)-2-fluoro-1,2,3,5,6,7-hexahydropyrrolizin-8-yl]methoxy]pyrazolo[4,3-f]quinazolin-4-yl]naphthalen-2-ol (example 1) (6 mg, 0.0095 mmol, 46% yield) as very pale yellow glassy solid.

UPLC-MS (ES⁺, Method 1): 3.04 min, m/z 629.6 [M+H]⁺.

¹H NMR (400 MHz, DMSO-d6) δ/ppm: 9.79 (s, 1H), 8.48 (s, 1H), 7.78-7.71 (m, 1H), 7.35-7.26 (m, 3H), 6.97 (d, J=1.9 Hz, 1H), 5.39-5.19 (m, 1H), 4.16-4.00 (m, 5H), 3.90-3.73 (m, 8H), 3.16-3.03 (m, 2H), 3.02 (br s, 1H), 2.88-2.79 (m, 1H), 2.40-2.35 (m, 1H), 2.21-2.14 (m, 1H), 2.10-1.96 (m, 5H), 1.88-1.75 (m, 3H), 0.53 (t, J=7.2 Hz, 3H).

The following examples in table 17 were made by analogy with example 1 (scheme 1) starting from the intermediate and building blocks for step A as described in the table The catalyst Pd(dtbpf)Cl₂ could be substituted for cataCXium A Pd G3 in step A.

TABLE 17

| | | UPLC- | |
|---|---|---|---|
| | | MS | |
| | | (ES⁺): | |
| Step A, intermediate + | | min, | ¹H NMR (400 MHz, |
| building block | Example Structure/Name | m/z | DMSO-d₆) δ/ppm |
| Intermediate I-4, and 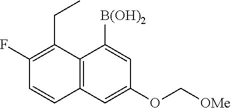 [8-ethyl-7-fluoro-3- (methoxymethoxy) naphthalen-1-yl]boronic acid | Example 6 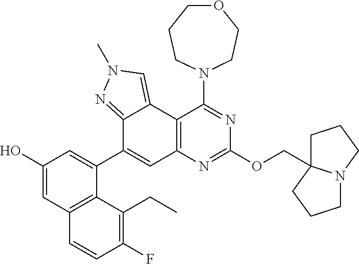 [SMILES: CCc1c(F)ccc2cc(O)cc(-c3cc4nc(OCC56CCCN5CCC6)nc(N5CCCOCC5)c4c4cn(C)nc34)c12] | 1.05 min, 611.3 [M + H]⁺ (Meth- od 4) | 8.48 (s, 1H), 8.23 (s, 1H), 7.76-7.72 (m, 1H), 7.34-7.28 (m, 3H), 6.99 (d, J = 4.0 Hz, 1H), 4.22-4.15 (m, 2H), 4.07 (s, 3H), 3.87-3.76 (m, 8H), 3.13-3.07 (m, 2H), 2.75-2.69 (m, 2H), 2.42-2.33 (m, 1H), 2.11-1.94 (m, 5H), 1.92-1.78 (m, 4H), 1.72-1.65 (m, 2H), 0.53 (t, J = 8.0 Hz, 3H). |
| | Example 6 | | |
| | 5-ethyl-6-fluoro-4-[7- | | |
| | (1,2,3,5,6,7- | | |
| | hexahydropyrrolizin-8- | | |
| | ylmethoxy)-2-methyl-9-(1,4- | | |
| | oxazepan-4-yl)pyrazolo[4,3- | | |
| | f]quinazolin-4-yl]naphthalen-2-ol | | |
| Intermediate I-6 and 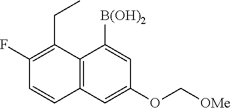 [8-ethyl-7-fluoro-3- (methoxymethoxy) naphthalen-1-yl]boronic acid | Example 7 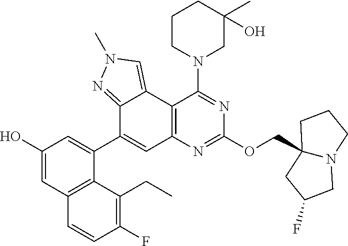 [SMILES: CCc1c(F)ccc2cc(O)cc(-c3cc4nc(OC[C@@]56CCCN5C[C@H](F)C6)nc(N5CCCC(C)(O)C5)c4c4cn(C)nc34)c12] Example 7 | 1.22 min, 643.3 [M + H]⁺ (Meth- od 4) | 9.80 (d, J = 2.4 Hz, 1H), 8.61 (d, J = 18.6 Hz, 1H), 7.74 (m, 1H), 7.42-7.18 (m, 3H), 6.98 (m, 1H), 5.31 (d, J = 54.1 Hz, 1H), 4.64 (d, J = 9.2 Hz, 1H), 4.16 (s, 1H), 4.08 (s, 4H), 3.55 (s, 1H), 3.50-3.39 (m, 2H), 3.12 (m, 3H), 2.87 (s, 1H), 2.41-2.31 (m, 1H), 2.23-1.95 (m, 6H), 1.93-1.55 (m, 6H), 1.08 (m, 3H), 0.53 (t, J = 7.3 Hz, 3H). |
| | 1-[4-(8-ethyl-7-fluoro-3- | | |
| | hydroxy-1-naphthyl)-2-methyl- | | |
| | 7-[[rac-(2R,8S)-2-fluoro- | | |
| | 1,2,3,5,6,7- | | |
| | hexahydropyrrolizin-8- | | |
| | yl]methoxy]pyrazolo[4,3- | | |
| | f]quinazolin-9-yl]-3-methyl- | | |
| | piperidin-3-ol | | |
| Intermediate I-8 and 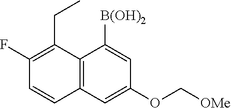 [8-ethyl-7-fluoro-3- (methoxymethoxy)naphthalen- 1-yl]boronic acid | Example 8 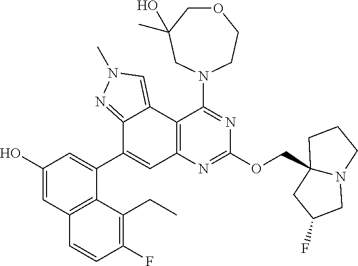 [SMILES: CCc1c(F)ccc2cc(O)cc(-c3cc4nc(OC[C@@]56CCCN5C[C@H](F)C6)nc(N5CCOCC(C)(O)C5)c4c4cn(C)nc34)c12] Example 8 | 2.95 min, 659.5 [M + H]⁺ (Meth- od 3) | δ 9.82-9.80 (d, J = 8.4 Hz, 1H), 8.48 (s, 1H), 7.76-7.72 (dd, J = 9.1, 6.0 Hz, 1H), 7.36-7.28 (m, 3H), 7.00-6.98 (dd, J = 6.2, 2.3 Hz, 1H), 5.56-5.53 (d, J = 14.7 Hz, 1H), 5.35- 5.21 (m, 1H), 4.15- 4.03 (m, 6H), 3.95- 3.70 (m, 6H), 3.58- 3.40 (m, 2H), 3.11- 3.02 (m, 3H), 2.88- 2.82 (m, 1H), 2.41- 2.32 (m, 1H), 2.18- 2.01 (m, 4H), 1.85- 1.78 (m, 3H), 1.05 (s, 1H), 0.87 (s, 2H), 0.57-0.49 (m, 3H). |
| | 4-[4-(8-ethyl-7-fluoro-3- | | |
| | hydroxy-1-naphthyl)-2-methyl- | | |
| | 7-[[(2R,8S)-2-fluoro- | | |
| | 1,2,3,5,6,7- | | |
| | hexahydropyrrolizin-8- | | |
| | yl]methoxy]pyrazolo[4,3-f] | | |
| | quinazolin-9-yl]-6-methyl-1,4- | | |
| | oxazepan-6-ol | | |
| Intermediate I-10 and 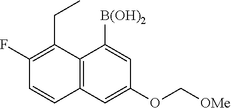 [8-ethyl-7-fluoro-3- (methoxymethoxy)naphthalen- 1-yl]boronic acid | Example 9 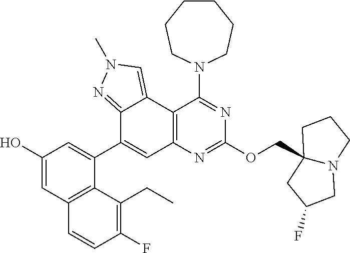 [SMILES: CCc1c(F)ccc2cc(O)cc(-c3cc4nc(OC[C@@]56CCCN5C[C@H](F)C6)nc(N5CCCCCC5)c4c4cn(C)nc34)c12] Example 9 | 3.02 min, 627.5 [M + H]⁺ (Meth- od 3) | 9.79 (s, 1H), 8.42 (s, 1H), 7.74 (dd, J = 9.1, 6.1 Hz, 1H), 7.33-7.26 (m, 3H), 6.98 (d, J = 2.6 Hz, 1H), 5.37- 5.21 (m, 1H), 4.12 (t, J = 9.6 Hz, 1H), 4.06 (s, 4H), 3.75 (m, 4H), 3.15- 3.02 (m, 3H), 2.84 (m, 1H), 2.36 (m, 1H), 2.18-2.00 (m, 4H), 1.88- 1.71 (m, 7H), 1.62 (m, 4H), 0.54- 0.48 (m, 3H). |
| | 4-[9-(azepan-1-yl)-2-methyl-7- | | |
| | [[(2R,8S)-2-fluoro-1,2,3,5,6,7- | | |
| | hexahydropyrrolizin-8- | | |
| | yl]methoxy]pyrazolo[4,3-f] | | |
| | quinazolin-4-yl]-5-ethyl-6- | | |
| | fluoro-naphthalen-2-ol | | |
| Intermediate I-12 and 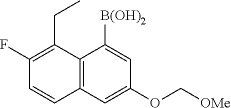 [8-ethyl-7-fluoro-3- (methoxymethoxy)naphthalen- 1-yl]boronic acid | Example 10 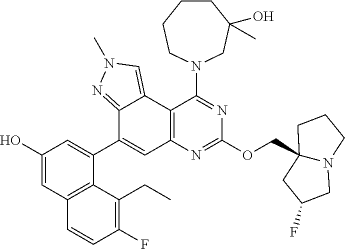 [SMILES: CCc1c(F)ccc2cc(O)cc(-c3cc4nc(OC[C@@]56CCCN5C[C@H](F)C6)nc(N5CCCCC(C)(O)C5)c4c4cn(C)nc34)c12] Example 10 1-[4-(8-ethyl-7-fluoro-3- hydroxy-1-naphthyl)-2-methyl- 7-[[(2R,8S)-2-fluoro-1,2,3,5,6,7- hexahydropyrrolizin-8-yl]methoxy]pyrazolo[4,3-f] quinazolin-9-yl]-3-methyl-azepan-3-ol | 1.50 min, 657.3 [M + H]⁺ (Meth- od 4) | 8.40 (d, J = 2.3 Hz, 1H), 7.74 (dd, J = 9.0, 6.0 Hz, 1H), 7.36-7.24 (m, 3H), 6.99 (dd, J = 4.4, 2.7 Hz, 1H), 5.35 (s, 1H), 4.17-4.02 (m, 5H), 3.80 (dd, J = 14.7, 7.9 Hz, 2H), 3.65 (dd, J = 17.9, 14.7 Hz, 2H), 3.12- 3.06 (m, 2H), 3.02 (s, 1H), 2.83 (d, J = 7.5 Hz, 1H), 2.34 (dd, J = 7.0, 3.0 Hz, 1H), 2.17-1.95 (m, 5H), 1.87-1.73 (m, 5H), 1.67-1.59 (m, 2H), 1.48 (s, 1H), 1.00 (s, 1H), 0.83 (s, 2H), 0.56-0.48 (m, 3H). |
| I-14 and 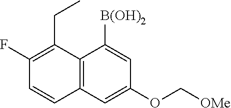 [8-ethyl-7-fluoro-3- (methoxymethoxy) naphthalen-1-yl] boronic acid | Example 13 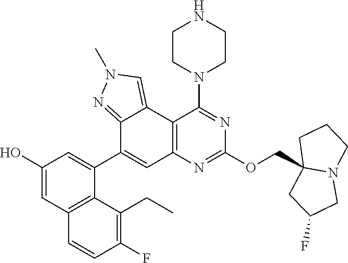 [SMILES: CCc1c(F)ccc2cc(O)cc(-c3cc4nc(OC[C@@]56CCCN5C[C@H](F)C6)nc(N5CCNCC5)c4c4cn(C)nc34)c12] | 0.86 min, 614.3 [M + H]⁺ (Meth- od 4) | 8.47 (s, 1H), 8.24 (s, 1H), 7.74 (dd, J = 9.0, 6.1 Hz, 1H), 7.38 (s, 1H), 7.36-7.25 (m, 2H), 7.23 (s, 1H) 6.97 (s, 1H), 5.29 (d, J = 54.4 Hz, 1H), 4.21-4.01 (m, 6H), 3.19-2.97 (m, 9H), 2.83 (q, J = 8.0 Hz, 1H), 2.43-2.28 (m, 1H), 2.29-1.62 (m, 8H), 0.53 (t, J = 7.3 Hz, 3H). |
| | Example 13 | | |
| | 5-ethyl-6-fluoro-4-[2-methyl-9- | | |
| | piperazin-1-yl-7-[[(2R,8S)-2- | | |
| | fluoro-1,2,3,5,6,7- | | |
| | hexahydropyrrolizin-8- | | |
| | yl]methoxy]pyrazolo[4,3- | | |
| | f]quinazolin-4-yl]naphthalen-2- | | |
| | ol | | |
| I-16 and 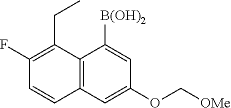 [8-ethyl-7-fluoro-3- (methoxymethoxy) naphthalen-1-yl]boronic acid | Example 14 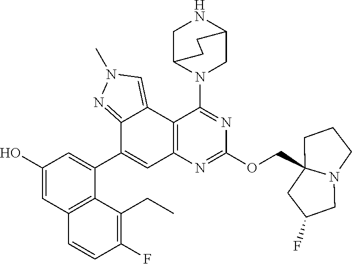 [SMILES: CCc1c(F)ccc2cc(O)cc(-c3cc4nc(OC[C@@]56CCCN5C[C@H](F)C6)nc(N5CC6CCC5CN6)c4c4cn(C)nc34)c12] Example 14 | 0.99 min, 640.3 [M + H]⁺ (Meth- od 4) | 8.49 (d, J = 17.7 Hz, 1H), 7.74 (dd, J = 9.1, 6.0 Hz, 1H), 7.34-7.28 (m, 3H), 6.98 (t, J = 2.6 Hz, 1H), 5.28 (d, J = 54.1 Hz, 1H), 4.47 (s, 1H), 4.14-4.01 (m, 6H), 3.84-3.70 (m, 2H), 3.51-3.25 (m, 3H), 3.15-3.02 (m, 3H), 2.83 (m, 1H), 2.43-2.29 (m, 2H), 2.15-1.98 (m, 7H), 1.86-1.78 (m, 3H), 0.52 (m, 4H). |
| | 4-[9-(2,5- | | |
| | diazabicyclo[2.2.2]octan-2-yl)- | | |
| | 2-methyl-7-[[(2R,8S)-2-fluoro- | | |
| | 1,2,3,5,6,7- | | |
| | hexahydropyrrolizin-8- | | |
| | yl]methoxy]pyrazolo[4,3- | | |
| | f]quinazolin-4-yl]-5-ethyl-6- | | |
| | fluoro-naphthalen-2-ol | | |
| I-18 and 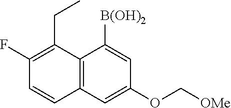 [8-ethyl-7-fluoro-3- (methoxymethoxy) naphthalen-1-yl]boronic acid | Example 15 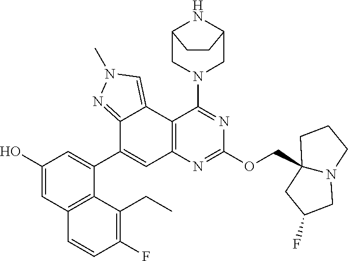 [SMILES: CCc1c(F)ccc2cc(O)cc(-c3cc4nc(OC[C@@]56CCCN5C[C@H](F)C6)nc(N5CC6CCC(C5)N6)c4c4cn(C)nc34)c12] Example 15 | 0.74 min, 640.3 [M + H]⁺ (Meth- od 4) | 8.25 (s, 2H), 7.73 (dd, J = 9.0, 6.0 Hz, 1H), 7.35-7.25 (m, 3H), 6.98 (d, J = 2.5 Hz, 1H), 5.28 (d, J = 53.7 Hz, 1H), 4.13 (t, J = 9.6 Hz, 3H), 4.07 (s, 3H), 4.05- 4.01 (m, 1H), 3.94- 3.89 (m, 1H), 3.81 (s, 2H), 3.69 (d, J = 12.0 Hz, 1H), 3.60 (d, J = 13.6 Hz, 1H), 3.15-3.02 (m, 3H), 2.87-2.79 (m, 1H), 2.44-2.29 (m, 1H), 2.17-2.06 (m, 2H), 2.03-1.93 (m, 2H), 1.87-1.73 (m, 6H), 0.53 (t, J = 7.2 Hz, 3H). |
| | 4-[9-[(3,8- | | |
| | diazabicyclo[3.2.1]octan-3-yl]- | | |
| | 7-[[(2R,8S)-2-fluoro- | | |
| | 1,2,3,5,6,7- | | |
| | hexahydropyrrolizin-8-yl]methoxy]- | | |
| | 2-methyl-pyrazolo[4,3-f]quinazolin-4-yl]- | | |
| | 5-ethyl-6-fluoro-naphthalen-2-ol | | |
| I-20 and 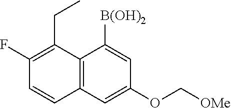 [8-ethyl-7-fluoro-3- (methoxymethoxy) naphthalen-1-yl]boronic acid | Example 17 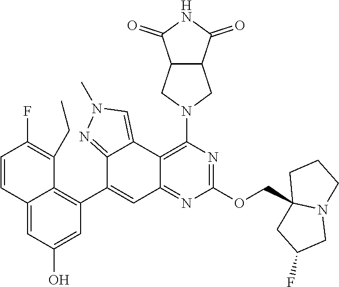 [SMILES: CCc1c(F)ccc2cc(O)cc(-c3cc4nc(OC[C@@]56CCCN5C[C@H](F)C6)nc(N5CC6C(=O)NC(=O)C6C5)c4c4cn(C)nc34)c12] Example 17 | 1.18 min, 668.2 [M + H]⁺ (Meth- od 4) | 11.24 (s, 1H), 10.71 (s, 1H), 9.85 (s, 1H), 8.35 (s, 1H), 7.75 (dd, J = 9.1, 6.0 Hz, 1H), 7.38 (s, 1H), 7.33-7.28 (m, 2H), 6.99 (d, J = 2.6 Hz, 1H), 5.57 (d, J = 51.2 Hz, 1H), 4.53 (s, 1H), 4.40-4.34 (m, 1H), 4.18 (dd, J = 11.8, 6.9 Hz, 1H), 4.08 (s, 3H), 3.81 (s, 1H), 3.69 (s, 2H), 3.63-3.50 (m, 5H), 2.35-2.12 (m, 5H), 2.07-1.96 (m, 3H), 1.55-1.43 (m, 1H), 0.49 (t, J = 7.3 Hz, 3H). Exchangeable proton missing. |
| | 5-[4-(8-ethyl-7-fluoro-3- | | |
| | hydroxy-1-naphthyl)-2-methyl- | | |
| | 7-[[(2R,8S)-2-fluoro- | | |
| | 1,2,3,5,6,7- | | |
| | hexahydropyrrolizin-8- | | |
| | yl]methoxy]pyrazolo[4,3- | | |
| | f]quinazolin-9-yl]-3a,4,6,6a- | | |
| | tetrahydropyrrolo[3,4-c]pyrrole- | | |
| | 1,3-dione | | |
| I-21 and 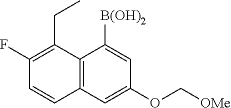 [8-ethyl-7-fluoro-3- (methoxymethoxy) naphthalen-1-yl]boronic acid | Example 18 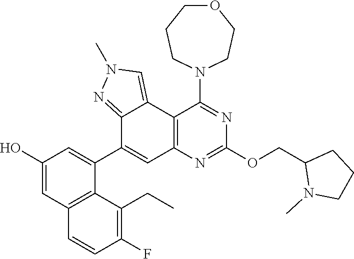 [SMILES: CCc1c(F)ccc2cc(O)cc(-c3cc4nc(OCC5CCCN5C)nc(N5CCCOCC5)c4c4cn(C)nc34)c12] Example 18 5-ethyl-6-fluoro-4-[2-methyl- 7-[(1-methylpyrrolidin-2- yl)methoxy]-9- (1,4-oxazepan-4-yl) pyrazolo[4,3-f]quinazolin- 4-yl]naphthalen-2-ol | 2.68 min, 585.5 [M + H]⁺ (Meth- od 4) | 10.87-9.94 (d, J = 372.6 Hz, 1H), 8.65 (s, 1H), 7.78-7.75 (dd, J = 9.0, 6.0 Hz, 1H), 7.56 (s, 1H), 7.35-7.31 (m, 2H), 7.01-7.00 (d, J = 2.6 Hz, 1H), 4.89-4.79 (m, J = 18.4, 10.4 Hz, 2H), 4.11 (s, 3H), 4.04-4.02 (m, J = 7.1 Hz, 2H), 3.96 (m, 1H), 3.92-3.86 (m, J = 6.4 Hz, 3H), 3.78-3.75 (m, J = 5.6 Hz, 2H), 3.62 (m, 2H), 3.15-3.10 (m, J = 11.3, 7.2 Hz, 1H), 2.97-2.96 (d, J = 4.3 Hz, 3H), 2.36- 2.28 (m, J = 12.4, 6.4 Hz, 2H), 2.10- 2.03 (m, 5H), 1.95- 1.87 (m, J = 13.8, 7.3 Hz, 1H), 0.56-0.52 (t, J = 7.3 Hz, 3H). |
| I-22 and 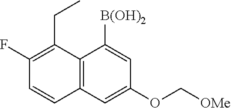 [8-ethyl-7-fluoro-3- (methoxymethoxy) naphthalen-1-yl]boronic acid | Example 19 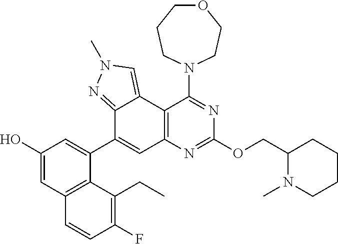 [SMILES: CCc1c(F)ccc2cc(O)cc(-c3cc4nc(OCC5CCCCN5C)nc(N5CCCOCC5)c4c4cn(C)nc34)c12] Example 19 | 2.85 min, 599.5 [M + H]⁺ (Meth- od 4) | 8.48 (s, 1H), 7.74 (dd, J = 9.0, 6.0 Hz, 1H), 7.33-7.27 (m, 3H), 6.98 (d, J = 2.6 Hz, 1H), 4.52-4.43 (m, 1H), 4.37-4.28 (m, 1H), 4.07 (s, 3H), 3.86-3.76 (m, 9H), 2.79 (d, J = 11.7 Hz, 1H), 2.39-2.29 (m, 5H), 2.10-2.01 (m, 4H), 1.78-1.68 (m, 2H), 1.53-1.35 (m, 3H), 0.53 (t, J = 7.3 Hz, 3H). Exchangeable proton not seen. |
| | 5-ethyl-6-fluoro-4-[2-methyl-7- | | |
| | [(1-methyl-2- | | |
| | piperidyl)methoxy]-(1,4- | | |
| | oxazepan-4-yl)pyrazolo[4,3- | | |
| | f]quinazolin-4-yl]naphthalen-2-ol | | |
| I-24 and 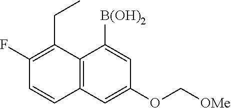 [8-ethyl-7-fluoro-3- (methoxymethoxy) naphthalen-1-yl]boronic acid | Example 20 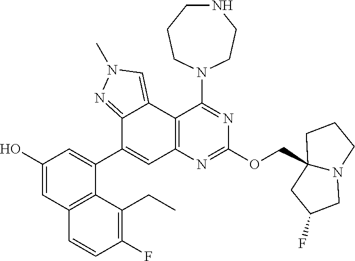 [SMILES: CCc1c(F)ccc2cc(O)cc(-c3cc4nc(OC[C@@]56CCCN5C[C@H](F)C6)nc(N5CCCNCC5)c4c4cn(C)nc34)c12] Example 20 | 3.28 min, 628.5 [M + H]⁺ (Meth- od 4) | 8.52 (s, 1H), 7.74 (dd, J = 9.1, 6.0 Hz, 1H), 7.34-7.28 (m, 3H), 6.98 (d, J = 2.6 Hz, 1H), 5.29 (d, J = 54.2 Hz, 1H), 4.14- 4.10 (m, 1H), 4.07 (s, 3H), 4.04-4.01 (m, 1H), 3.88-3.75 (m, 5H), 3.29-3.27 (m, 2H), 3.12-3.08 (m, 4H), 3.02 (s, 1H), 2.87-2.81 (m, 1H), 2.40-2.33 (m, 1H), 2.17-1.99 (m, 6H), 1.86-1.78 (m, 3H), 0.53 (t, J = 7.2 Hz, 3H). Exchangeable proton not seen. |
| | 4-[9-(1,4-diazepan-1-yl)-2- | | |
| | methyl-7-[[(2R,8S)-2-fluoro- | | |
| | 1,2,3,5,6,7- | | |
| | hexahydropyrrolizin-8- | | |
| | yl]methoxy]pyrazolo[4,3- | | |
| | f]quinazolin-4-yl]-5-ethyl-6- | | |
| | fluoro-naphthalen-2-ol | | |
| I-26 and 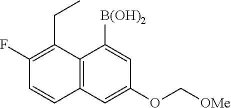 [8-ethyl-7-fluoro-3- (methoxymethoxy) naphthalen-1-yl]boronic acid | Example 21 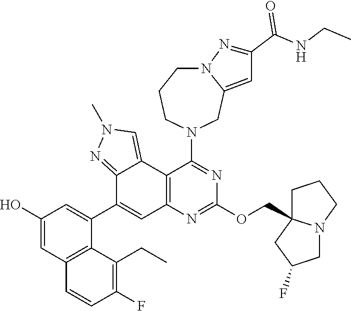 [SMILES: CCNC(=O)c1cc2n(n1)CCCN(c1nc(OC[C@@]34CCCN3C[C@H](F)C4)nc3cc(-c4cc(O)cc5ccc(F)c(CC)c45)c4nn(C)cc4c13)C2] Example 21 | 1.27 min, 736.2 [M + H]⁺ (Meth- od 4). | 9.81 (br s, 1H), 8.49 (s, 1H), 8.15 (s, 1H), 7.99 (t, J = 5.9 Hz, 1H), 7.76-7.73 (m, 1H), 7.35-7.28 (m, 3H), 6.99 (d, J = 2.5 Hz, 1H), 6.26 (d, J = 2.5 Hz, 1H), 5.26 (d, J = 53.8 Hz, 1H), 4.94 (d, J = 15.9 Hz, 1H), 4.80 (d, J = 16.0 Hz, 1H), 4.56-4.46 (m, 2H), 4.08 (s, 3H), 4.04-3.93 (m, 2H), 3.87 (t, J = 21.6 Hz, 1H), 3.23-3.16 (m, 2H), 3.14-2.97 (m, 4H), 2.84-2.80 (m, 1H), 2.43-2.38 (m, 1H), 2.31-2.19 (m, 1H), 2.11-2.07 (m, 2H), 2.04-1.97 (m, 2H), 1.88-1.69 (m, 3H), 1.05 (t, J = 7.1 Hz, 3H), 0.56 (t, J = 7.3 Hz, 3H) |
| | N-ethyl-5-[4-(8-ethyl-7-fluoro- | | |
| | 3-hydroxy-1-naphthyl)-2- | | |
| | methyl-7-[[(2R,8S)-2-fluoro- | | |
| | 1,2,3,5,6,7- | | |
| | hexahydropyrrolizin-8- | | |
| | yl]methoxy]pyrazolo[4,3- | | |
| | f]quinazolin-9-yl]-4,6,7,8- | | |
| | tetrahydropyrazolo[1,5- | | |
| | a][1,4]diazepine-2- | | |
| | carboxamide | | |
| I-28 and 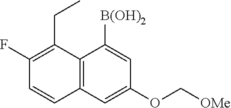 [8-ethyl-7-fluoro-3- (methoxymethoxy) naphthalen-1-yl]boronic acid | Example 22 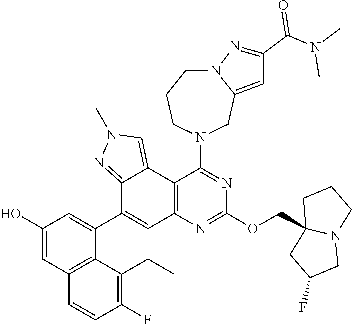 [SMILES: CCc1c(F)ccc2cc(O)cc(-c3cc4nc(OC[C@@]56CCCN5C[C@H](F)C6)nc(N5CCCn6nc(C(=O)N(C)C)cc6C5)c4c4cn(C)nc34)c12] Example 22 | 1.27 min, 736.2 [M + H]⁺ (Meth- od 4) | 9.81 (br s, 1H), 8.49 (s, 1H), 7.76-7.73 (m, 1H), 7.35-7.28 (m, 3H), 6.99 (d, J = 2.6 Hz, 1H), 6.23 (s, 1H), 5.28 (d, J = 54.2 Hz, 1H), 4.94 (d, J = 15.9 Hz, 1H), 4.79 (d, J = 15.9 Hz, 1H), 4.56-4.47 (m, 2H), 4.08 (s, 3H), 4.06- 3.97 (m, 2H), 3.90- 3.85 (m, 1H), 3.25 (s, 3H), 3.16-3.03 (m, 3H), 2.92 (s, 3H), 2.87-2.80 (m, 1H), 2.42-2.36 (m, 1H), 2.31-1.95 (m, 7H), 1.91-1.73 (m, 3H), 0.55 (t, J = 7.3 Hz, 3H). |
| | 5-[4-(8-ethyl-7-fluoro-3- | | |
| | hydroxy-1-naphthyl)-2-methyl- | | |
| | 7-[[(2R,8S)-2-fluoro- | | |
| | 1,2,3,5,6,7- | | |
| | hexahydropyrrolizin-8- | | |
| | yl]methoxy]pyrazolo[4,3- | | |
| | f]quinazolin-9-yl]-N,N-dimethyl- | | |
| | 4,6,7,8-tetrahydropyrazolo[1,5- | | |
| | a][1,4-]diazepine-2-carboxamide | | |
| I-30 and 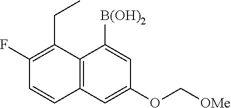 [8-ethyl-7-fluoro-3- (methoxymethoxy) naphthalen-1-yl]boronic acid | Example 23 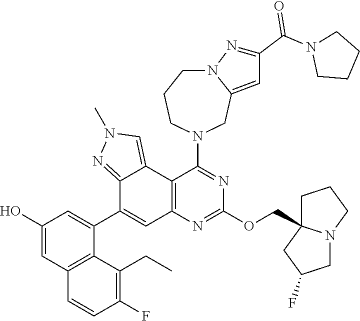 [SMILES: CCc1c(F)ccc2cc(O)cc(-c3cc4nc(OC[C@@]56CCCN5C[C@H](F)C6)nc(N5CCCn6nc(C(=O)N7CCCC7)cc6C5)c4c4cn(C)nc34)c12] Example 23 | 3.02 min, 762.6 [M + H]⁺ (Meth- od 4) | 9.86 (s, 1H), 8.49 (s, 1H), 7.76-7.72 (dd, J = 9.1, 6.0 Hz, 1H), 7.35-7.29 (m, 3H), 7.02-7.01 (d, J = 2.5 Hz, 1H), 6.29 (s, 1H), 5.34-5.20 (d, J = 54.5 Hz, 1H), 4.98- 4.78 (m, 2H), 4.53- 4.47 (d, J = 7.0 Hz, 2H), 4.08-3.80 (m, 9H), 3.43-3.40 (t, J = 6.7 Hz, 2H), 3.09- 3.02 (d, J = 28.7 Hz, 2H), 2.86-2.81 (t, J = 11.9 Hz, 1H), 2.40- 1.79 (m, 15H), 0.58- 0.54 (t, J = 7.2 Hz, 3H). |
| | [5-[4-(8-ethyl-7-fluoro-3- | | |
| | hydroxy-1-naphthyl)-2-methyl- | | |
| | 7-[[(2R,8S)-2-fluoro- | | |
| | 1,2,3,5,6,7-hexahydropyrrolizin-8- | | |
| | yl]methoxy]pyrazolo-[4,3- | | |
| | f]quinazolin-9-yl]4,6,7,8- | | |
| | tetrahydropyrazolo[1,5-a] | | |
| | [1,4]diazepin-2-yl]-pyrrolidin- | | |
| | 1-yl-methanone | | |
| I-32 and 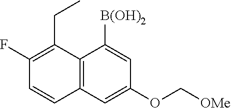 [8-ethyl-7-fluoro-3- (methoxymethoxy) naphthalen-1-yl]boronic acid | Example 24 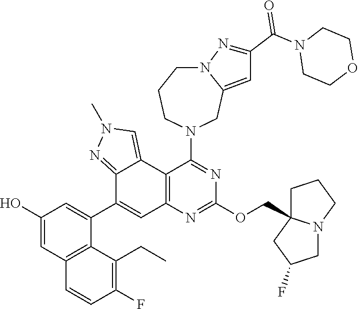 [SMILES: CCc1c(F)ccc2cc(O)cc(-c3cc4nc(OC[C@@]56CCCN5C[C@H](F)C6)nc(N5CCCn6nc(C(=O)N7CCOCC7)cc6C5)c4c4cn(C)nc34)c12] Example 24 | 1.18 min, 778.3 [M + H]⁺ (Meth- od 4) | 8.48 (s, 1H), 7.75- 7.72 (m, 1H), 7.35- 7.27 (m, 3H), 7.0 (d, J = 2.4 Hz, 1H), 6.27 (s, 1H), 5.26 (d, J = 56.0 Hz, 1H), 4.96- 4.77 (m, 2H), 4.53- 4.50 (m, 2H), 4.07 (s, 3H), 4.05-3.84 (m, 6H), 3.59 (s, 6H), 3.11-2.99 (m, 3H), 2.84-2.78 (m, 1H), 2.42-2.36 (m, 1H), 2.26-2.19 (m, 1H), 2.12-1.97 (m, 5H), 1.83-1.72 (m 3H), 0.55 (t, J = 8.0 Hz, 3H). Exchangeable proton not seen. |
| | [5-[4-(8-ethyl-7-fluoro-3- | | |
| | hydroxy-1-naphthyl)-2-methyl- | | |
| | 7-[[(2R,8S)-2-fluoro-1,2,3,5,6,7- | | |
| | hexahydropyrrolizin-8- | | |
| | yl]methoxy]pyrazolo[4,3-f] | | |
| | quinazolin-9-yl]-4,6,7,8- | | |
| | tetrahydropyrazolo[1,5- | | |
| | a][1,4]diazepin-2-yl]- | | |
| | morpholino-methanone | | |
| I-34 and 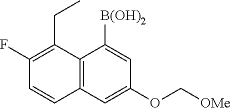 [8-ethyl-7-fluoro-3- (methoxymethoxy) naphthalen-1-yl]boronic acid | Example 25 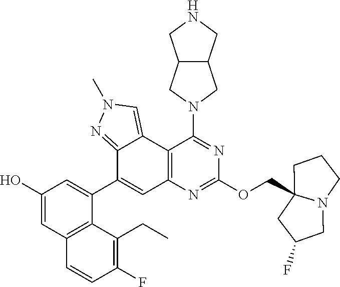 [SMILES: CCc1c(F)ccc2cc(O)cc(-c3cc4nc(OC[C@@]56CCCN5C[C@H](F)C6)nc(N5CC6CNCC6C5)c4c4cn(C)nc34)c12] Example 25 | 0.74 min, 640.3 [M + H]⁺ (Meth- od 4) | 8.46 (s, 1H), 7.74 (dd, J = 9.1, 6.0 Hz, 1H), 7.34 (s, 1H), 7.32-7.26 (m, 2H), 6.99 (d, J = 2.5 Hz, 1H), 5.30 (d, J = 53.8 Hz, 1H), 4.16-4.07 (m, 4H), 4.06-3.95 (m, 2H), 3.80-3.74 (m, 1H), 3.56-3.50 (m, 1H), 3.37-3.26 (m, 3H), 3.22-3.00 (m, 7H), 2.87-2.81 (m, 1H), 2.41-2.32 (m, 1H), 2.23-1.87 (m, 4H), 1.87-1.75 (m, 3H), 0.53 (t, J = 7.2 Hz, 3H). Exchangeable protons not seen (2H). |
| | 4-(2,7-diazaspiro[4.4]nonan-2- | | |
| | yl)-6-(8-ethyl-7-fluoro-3- | | |
| | hydroxy-1-naphthyl)-2- | | |
| | [[(2R,8S)-2-fluoro-1,2,3,5,6,7- | | |
| | hexahydropyrrolizin-8- | | |
| | yl]methoxy]-7H-pyrrolo[3,4- | | |
| | d]pyrimidin-5-one | | |
| I-35 and 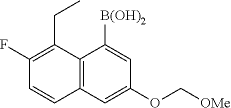 [8-ethyl-7-fluoro-3- (methoxymethoxy) naphthalen-1-yl]boronic acid | Example 26 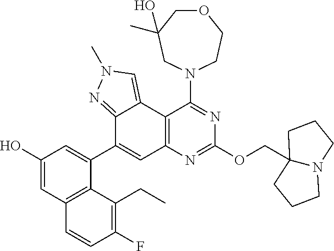 [SMILES: CCc1c(F)ccc2cc(O)cc(-c3cc4nc(OCC56CCCN5CCC6)nc(N5CCOCC(C)(O)C5)c4c4cn(C)nc34)c12] Example 26 | 1.19 min, 641.3 [M + H]⁺ (Meth- od 4) | 10.20 (s, 1H), 8.53 (s, 1H), 7.75 (dd, J = 9.1, 6.0 Hz, 1H), 7.40 (d, J = 7.9 Hz, 1H), 7.35-7.26 (m, 2H), 6.99 (t, J = 3.0 Hz, 1H), 4.62-4.47 (m, 2H), 4.09 (d, J = 3.6 Hz, 3H), 4.01-3.87 (m, 4H), 3.84-3.71 (m, 4H), 3.59-3.41 (m, 4H), 3.30-3.18 (m, 2H), 2.41-2.31 (m, 1H), 2.18 (dd, J = 11.8, 5.5 Hz, 2H), 2.14-1.95 (m, 6H), 0.96 (d, J = 71.5 Hz, 3H), 0.53 (dt, J = 10.3, 7.3 Hz, 3H). |
| | 4-[4-(8-ethyl-7-fluoro-3- | | |
| | hydroxy-1-naphthyl)-7- | | |
| | (1,2,3,5,6,7- | | |
| | hexahydropyrrolizin-8- | | |
| | ylmethoxy)-2-methyl- | | |
| | pyrazolo[4,3-f]quinazolin-9-yl]- | | |
| | 6-methyl-1,4-oxazepan-6-ol | | |
| I-37 and 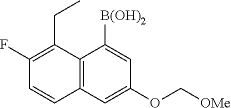 [8-ethyl-7-fluoro-3- (methoxymethoxy) naphthalen-1-yl]boronic acid | Example 27 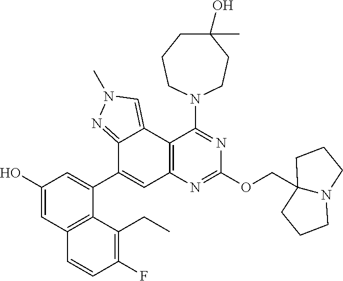 [SMILES: CCc1c(F)ccc2cc(O)cc(-c3cc4nc(OCC56CCCN5CCC6)nc(N5CCCC(C)(O)CC5)c4c4cn(C)nc34)c12] Example 27 | 2.68 min, 639.6 [M + H]⁺ (Meth- od 4) | 8.44 (d, J = 2.9 Hz, 1H), 8.23 (s, 1H), 7.73 (dd, J = 9.1, 6.0 Hz, 1H), 7.34-7.26 (m, 3H), 6.98 (d, J = 2.6 Hz, 1H), 4.11 (t, J = 3.7 Hz, 2H), 4.06 (d, J = 2.0 Hz, 3H), 3.98-3.85 (m, 2H), 3.80-3.69 (m, 3H), 3.02 (dd, J = 10.7, 5.6 Hz, 2H), 2.64 (dt, J = 11.0, 6.9 Hz, 2H), 2.40-2.33 (m, 1H), 2.15-2.04 (m, 1H), 2.04-1.89 (m, 4H), 1.79 (ddd, J = 28.0, 13.5, 6.9 Hz, 6H), 1.69-1.56 (m, 4H), 1.17 (d, J = 8.2 Hz, 3H), 0.51 (dt, J = 10.8, 7.3 Hz, 3H). |
| | 1-[4-(8-ethyl-7-fluoro-3- | | |
| | hydroxy-1-naphthyl)-7- | | |
| | (1,2,3,5,6,7- | | |
| | hexahydropyrrolizin-8- | | |
| | ylmethoxy)-2-methyl- | | |
| | pyrazolo[4,3-f]quinazolin-9-yl]- | | |
| | 4-methyl-azepan-4-ol | | |
| I-38 and 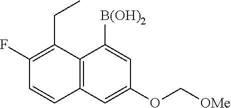 [8-ethyl-7-fluoro-3- (methoxymethoxy) naphthalen-1-yl]boronic acid | Example 28 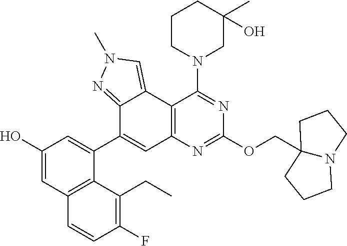 Example 28 | 1.22 min, 643.3 [M + H]⁺ (Meth- od 4) | 10.25 (s, 1H), 8.65 (d, J = 16.9 Hz, 1H), 7.75 (dd, J = 9.0, 6.0 Hz, 1H), 7.40 (d, J = 3.4 Hz, 1H), 7.34- 7.29 (m, 2H), 6.98 (t, J = 2.6 Hz, 1H), 4.57 (t, J = 12 Hz, 2H), 4.10 (s, 3H), 3.72 (s, 1H), 3.55-3.49 (m, 4H), 3.41-3.21 (m, 4H), 2.36 (dd, J = 14.3, 7.7 Hz, 1H), 2.21-2.00 (m, 10H), 1.77-1.65 (m, 3H), 1.07 (d, J = 33.5 Hz, 3H), 0.55-0.50 (m, 3H). |
| | 1-[4-(8-ethyl-7-fluoro-3- | | |
| | hydroxy-1-naphthyl)-7- | | |
| | (1,2,3,5,6,7- | | |
| | hexahydropyrrolizin-8- | | |
| | ylmethoxy)-2-methyl- | | |
| | pyrazolo[4,3-f]quinazolin-9-yl]- | | |
| | 3-methyl-piperidin-3-ol | | |
| I-39 and 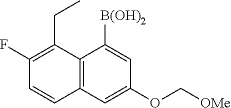 [8-ethyl-7-fluoro-3- (methoxymethoxy) naphthalen-1-yl]boronic acid | Example 29 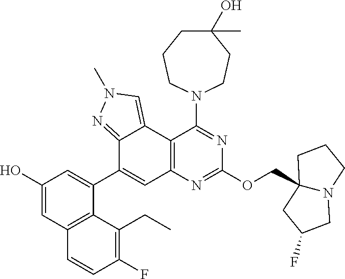 [SMILES: CCc1c(F)ccc2cc(O)cc(-c3cc4nc(OC[C@@]56CCCN5C[C@H](F)C6)nc(N5CCCC(C)(O)CC5)c4c4cn(C)nc34)c12] Example 29 1-[4-(8-ethyl-7-fluoro-3-hydroxy-1- naphthyl)-7-[[(2R,8S)-2-fluoro-1,2,3,5,6,7- hexahydropyrrolizin-8-yl]methoxy]-2-methyl- pyrazolo[4,3-f]quinazolin-9-yl]- 4-methyl-azepan-4-ol | 1.05 min, 657.4 [M + H]⁺ (Meth- od 4) | 8.46-8.42 (m, 1H), 8.19 (s, 1H), 7.73 (dd, J = 9.1, 6.0 Hz, 1H), 7.35-7.26 (m, 3H), 6.98 (d, J = 2.6 Hz, 1H), 5.29 (d, J = 54.4 Hz, 1H), 4.15- 4.10 (m, 1H), 4.06 (d, J = 2.1 Hz, 3H), 4.04-3.70 (m, 4H), 3.57 (ddd, J = 33.0, 14.3, 8.1 Hz, 3H), 3.11 (d, J = 11.2 Hz, 2H), 3.04 (s, 1H), 2.84 (q, J = 8.2 Hz, 1H), 2.37 (m, J = 9.5, 3.6, 2.5 Hz, 1H), 2.18-2.12 (m, 1H), 2.09-1.90 (m, 4H), 1.88-1.66 (m, 6H), 1.58 (t, J = 12.1 Hz, 1H), 1.17 (d, J = 8.2 Hz, 4H), 0.51 (m, J = 11.2, 7.2 Hz, 3H). |
| I-40 and 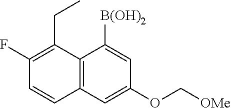 [8-ethyl-7-fluoro-3- (methoxymethoxy) naphthalen-1-yl]boronic acid | Example 30 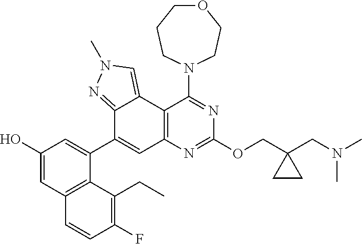 [SMILES: CCc1c(F)ccc2cc(O)cc(-c3cc4nc(OCC5(CN(C)C)CC5)nc(N5CCCOCC5)c4c4cn(C)nc34)c12] Example 30 4-[7-[[1- [(dimethylamino)methyl]cyclopropyl] methoxy]-2-methyl-9-(1,4-oxazepan-4-yl) pyrazolo[4,3-f]quinazolin-4-yl]-5-ethyl-6- fluoro-naphthalen-2-ol | 2.65 min, 599.5 [M + H]⁺ (Meth- od 4) | 10.07-9.99 (d, J = 32.2 Hz, 1H), 8.68 (s, 1H), 7.79-7.75 (dd, J = 9.1, 6.0 Hz, 1H), 7.60 (s, 1H), 7.36-7.32 (m, 2H), 7.01-7.00 (d, J = 2.6 Hz, 1H), 4.54-4.47 (m, J = 8.0 Hz, 2H), 4.12 (s, 3H), 4.08- 3.92 (m, 4H), 3.90- 3.84 (m, 2H), 3.77- 3.74 (t, J = 5.6 Hz, 2H), 3.25-3.23 (d, J = 5.4 Hz, 2H), 2.86- 2.85 (d, J = 4.4 Hz, 6H), 2.35-2.28 (m, 1H), 2.10 (m, 1H), 2.06-1.99 (m, J = 15.2, 7.9, 4.0 Hz, 2H), 0.94-0.90 (m, J = 3.9 Hz, 2H), 0.88-0.83 (m, J = 3.7 Hz, 2H), 0.56-0.52 (t, J = 7.3 Hz, 3H). |
| I-43 and 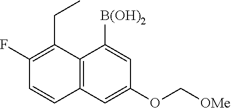 [8-ethyl-7-fluoro-3- (methoxymethoxy) naphthalen-1-yl]boronic acid | Example 31 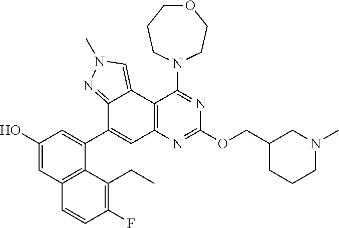 [SMILES: CCc1c(F)ccc2cc(O)cc(-c3cc4nc(OCC5CCCN(C)C5)nc(N5CCCOCC5)c4c4cn(C)nc34)c12] Example 31 | 0.86 min, 599.3 [M + H]⁺ (Meth- od 4) | 8.48 (s, 1H), 8.25 (s, 1H), 7.75-7.72 (m, 1H), 7.33-7.27 (m, 3H), 6.99 (d, J = 4.0 Hz, 1H), 4.22-4.19 (m, 2H), 4.07 (s, 3H), 3.91-3.75 (m, 8H), 2.94 (d, J = 12.0 Hz, 2H), 2.41-2.34 (m, 1H), 2.30 (s, 3H), 2.17-1.96 (m, 5H), 1.86-1.77 (m, 3H), 1.42-1.33 (m, 2H), 0.53 (t, J = 8.0 Hz, 3H). |
| | 5-ethyl-6-fluoro-4-[2-methyl-7- | | |
| | [(1-methyl-4- | | |
| | piperidyl)methoxy]-9-(1,4- | | |
| | oxazepan-4-yl)pyrazolo[4,3- | | |
| | f]quinazolin-4-yl]naphthalen-2-ol | | |
| I-44 and 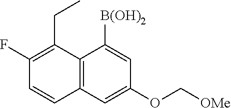 [8-ethyl-7-fluoro-3- (methoxymethoxy) naphthalen-1-yl]boronic acid | Example 32 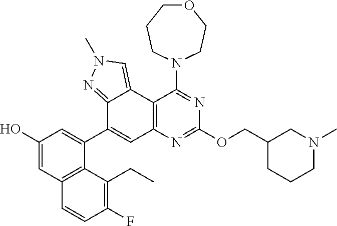 [SMILES: CCc1c(F)ccc2cc(O)cc(-c3cc4nc(OCC5CCCN(C)C5)nc(N5CCCOCC5)c4c4cn(C)nc34)c12] Example 32 | 0.20 min, 599.3 [M + H]⁺ (Meth- od 4) | 8.48 (s, 1H), 7.76- 7.72 (m, 1H), 7.33- 7.27 (m, 3H), 6.98 (d, J = 2.6 Hz, 1H), 4.31-4.26 (m, 1H), 4.21-4.15 (m, 1H), 4.07 (s, 3H), 3.94- 3.74 (m, 8H), 2.97 (d, J = 10.1 Hz, 1H), 2.79 (d, J = 11.2 Hz, 1H), 2.39-2.34 (m, 1H), 2.29 (s, 3H), 2.20-1.94 (m, 6H), 1.80-1.67 (m, 2H), 1.63-1.49 (m, 1H), 1.17-1.08 (m, 1H), 0.53 (t, J = 7.3 Hz, 3H). Exchangeable proton missing. |
| | 5-ethyl-6-fluoro-4-[2-methyl-7- | | |
| | [(1-methyl-3- | | |
| | piperidyl)methoxy]-9-(1,4- | | |
| | oxazepan-4-yl)pyrazolo[4,3- | | |
| | f]quinazolin-4-yl]naphthalen-2-ol | | |
| I-45 and 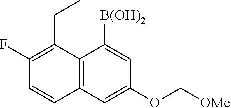 [8-ethyl-7-fluoro-3- (methoxymethoxy) naphthalen-1-yl]boronic acid | Example 40 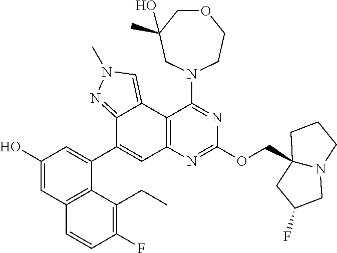 [SMILES: CCc1c(F)ccc2cc(O)cc(-c3cc4nc(OC[C@@]56CCCN5C[C@H](F)C6)nc(N5CCOC[C@@](C)(O)C5)c4c4cn(C)nc34)c12] Example 40 | 1.19 min, 659.1 [M + H]⁺ (Meth- od 4) | 9.87 (d, J = 6.5 Hz, 1H), 8.49 (s, 1H), 7.73 (dd, J = 9.0, 6.0 Hz, 1H), 7.37-7.26 (m, 3H), 7.01 (dd, J = 5.1, 2.6 Hz, 1H), 5.58-5.19 (m, 2H), 4.14-4.04 (m, 5H), 3.91-3.69 (m, 5H), 3.60-3.37 (m, 3H), 3.17-3.00 (m, 3H), 2.88-2.78 (m, 1H), 2.38-1.78 (m, 8H), 0.96 (d, J = 68.4 Hz, 3H), 0.53 (dt, J = 16.6, 7.2 Hz, 3H). |
| | (6S)-4-[4-(8-ethyl-7-fluoro-3- | | |
| | hydroxy-1-naphthyl)-2-methyl- | | |
| | 7-[[(2R,8S)-2-fluoro- | | |
| | 1,2,3,5,6,7- | | |
| | hexahydropyrrolizin-8- | | |
| | yl]methoxy]pyrazolo[4,3- | | |
| | f]quinazolin-9-yl]-6-methyl-1,4- | | |
| | oxazepan-6-ol | | |
| I-46 and 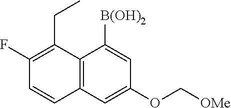 [8-ethyl-7-fluoro-3- (methoxymethoxy) naphthalen-1-yl]boronic acid | Example 41 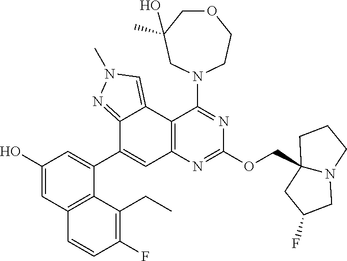 [SMILES: CCc1c(F)ccc2cc(O)cc(-c3cc4nc(OC[C@@]56CCCN5C[C@H](F)C6)nc(N5CCOC[C@](C)(O)C5)c4c4cn(C)nc34)c12] Example 41 (6R)-4-[4-(8-ethyl-7-fluoro-3- hydroxy-1-naphthyl)-2-methyl-7-[[(2R,8S)- 2-fluoro-1,2,3,5,6,7-hexahydropyrrolizin- 8-yl]methoxy]pyrazolo[4,3-f]quinazolin- 9-yl]-6-methyl-1,4-oxazepan-6-ol | 2.98 min, 659.5 [M + H]⁺ (Meth- od 4) | 9.79 (d, J = 8.9 Hz, 1H), 8.48 (s, 1H), 7.74 (dd, J = 9.1, 6.0 Hz, 1H), 7.38- 7.27 (m, 3H), 6.99 (dd, J = 7.0, 2.6 Hz, 1H), 5.61-5.18 (m, 2H), 4.18-4.02 (m, 6H), 3.94-3.84 (m, 2H), 3.73 (dd, J = 21.3, 14.1 Hz, 3H), 3.57-3.40 (m, 2H), 3.11 (t, J = 25.5 Hz, 3H), 2.85 (s, 1H), 2.35 (dd, J = 14.2, 6.4 Hz, 1H), 2.07 (dd, J = 27.3, 16.6 Hz, 4H), 1.81 (s, 3H), 0.96 (d, J = 68.7 Hz, 3H), 0.53 (dt, J = 18.3, 7.2 Hz, 3H). |
| I-48 and 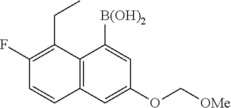 [8-ethyl-7-fluoro-3- (methoxymethoxy) naphthalen-1-yl]boronic acid | Example 42 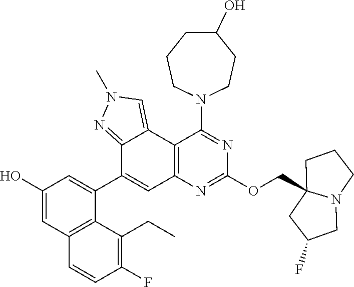 [SMILES: CCc1c(F)ccc2cc(O)cc(-c3cc4nc(OC[C@@]56CCCN5C[C@H](F)C6)nc(N5CCCC(O)CC5)c4c4cn(C)nc34)c12] | 0.98 min 643.1 [M + H]⁺ (Meth- od 4) | 9.83 (s, 1H), 8.44 (s, 1H), 7.73 (dd, J = 9.1, 6.0 Hz, 1H), 7.37-7.21 (m, 3H), 6.99 (d, J = 2.6 Hz, 1H), 5.32 (d, J = 54.0 Hz, 1H), 4.63 (dd, J = 5.9, 3.8 Hz, 1H), 4.06 (s, 5H), 3.85- 3.58 (m, 5H), 3.14 (d, J = 26.4 Hz, 3H), 2.89 (d, J = 13.9 Hz, 1H), 2.37-1.59 (m, 14H), 0.51 (td, J = 7.3, 3.2 Hz, 3H). |
| | Example 42 | | |
| | 1-[4-(8-ethyl-7-fluoro-3- | | |
| | hydroxy-1-naphthyl)-2-methyl- | | |
| | 7-[[(2R,8S)-2-fluoro- | | |
| | 1,2,3,5,6,7- | | |
| | hexahydropyrrolizin-8- | | |
| | yl]methoxy]pyrazolo[4,3- | | |
| | f]quinazolin-9-yl]azepan-4-ol | | |
| I-51 and 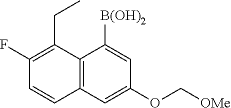 [8-ethyl-7-fluoro-3- (methoxymethoxy) naphthalen-1-yl]boronic acid | Example 44 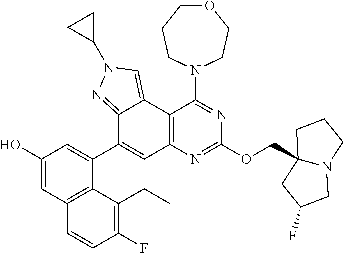 [SMILES: CCc1c(F)ccc2cc(O)cc(-c3cc4nc(OC[C@@]56CCCN5C[C@H](F)C6)nc(N5CCCOCC5)c4c4cn(C5CC5)nc34)c12] | 1.44 min, 655.3 [M + H]⁺ (Meth- od 4) | 9.80 (s, 1H), 8.47 (s, 1H), 7.74 (dd, J = 9.0, 6.0 Hz, 1H), 7.33-7.28 (m, 3H), 6.99 (d, J = 2.6 Hz, 1H), 5.29 (d, J = 54.1 Hz, 1H), 4.16-4.01 (m, 3H), 3.87-3.75 (m, 8H), 3.12 (d, J = 6.1 Hz, 2H), 3.05 (s, 1H), 2.85 (dd, J = 14.3, 6.7 Hz, 1H), 2.36-2.30 (m, 1H), 2.09-1.99 (m, 6H), 1.87-1.76 (m, 3H), 1.14-1.00 (m, 4H), 0.48 (t, J = 7.3 Hz, 3H). |
| | Example 44 | | |
| | 4-[2-cyclopropyl-9-(1,4- | | |
| | oxazepan-4-yl)-7-[[(2R,8S)-2- | | |
| | fluoro-1,2,3,5,6,7- | | |
| | hexahydropyrrolizin-8- | | |
| | yl]methoxy]pyrazolo[4,3- | | |
| | f]quinazolin-4-yl]-5-ethyl-6- | | |
| | fluoro-naphthalen-2-ol | | |
| I-54 and 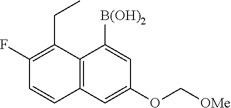 [8-ethyl-7-fluoro-3- (methoxymethoxy) naphthalen-1-yl]boronic acid | Example 45 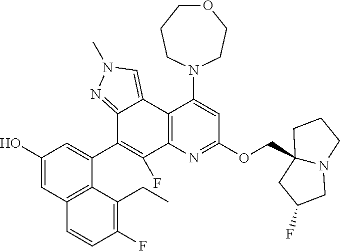 [SMILES: CCc1c(F)ccc2cc(O)cc(-c3c(F)c4nc(OC[C@@]56CCCN5C[C@H](F)C6)cc(N5CCCOCC5)c4c4cn(C)nc34)c12] | 3.22 min, 646.5 [M + H]⁺ (Meth- od 3) | 9.86 (s, 1H), 8.72 (s, 1H), 7.78-7.74 (dd, J = 9.0, 6.0 Hz, 1H), 7.34-7.30 (m, 2H), 7.01-7.00 (d, J = 2.6 Hz, 1H), 6.89 (s, 1H), 5.42-5.28 (m, 1H), 4.28-4.20 (m, 2H), 4.09 (s, 3H), 3.95- 3.90 (d, J = 22.3 Hz, 4H), 3.54-3.43 (m, 6H), 3.19-2.92 (d, J = 24.6 Hz, 2H), 2.38- 1.85 (m, 10H), 0.57- 0.53 (m, 3H). |
| | Example 45 | | |
| | 5-ethyl-6-fluoro-4-[5-fluoro-2- | | |
| | methyl-9-(1,4-oxazepan-4-yl)- | | |
| | 7-[[(2R,8S)-2-fluoro- | | |
| | 1,2,3,5,6,7- | | |
| | hexahydropyrrolizin-8- | | |
| | yl]methoxy]pyrazolo[4,3- | | |
| | f]quinolin-4-yl]naphthalen-2-ol | | |
| I-57 and 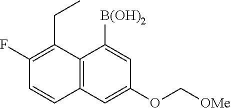 [8-ethyl-7-fluoro-3- (methoxymethoxy) naphthalen-1-yl]boronic acid | Example 46 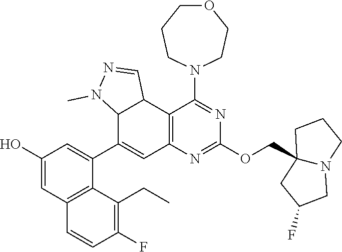 [SMILES: CCc1c(F)ccc2cc(O)cc(C3=Cc4nc(OC[C@@]56CCCN5C[C@H](F)C6)nc(N5CCCOCC5)c4C4C=NN(C)C34)c12] | 1.38 min, 629.3 [M + H]⁺ (Meth- od 4) | 10.00 (s, 1H), 8.27 (s, 1H), 7.81 (dd, J = 9.1, 6.0 Hz, 1H), 7.50 (s, 1H), 7.39-7.34 (m, 2H), 7.09 (t, J = 1.8 Hz, 1H), 5.29 (d, J = 54.1 Hz, 1H), 4.13- 4.05 (m, 2H), 3.94- 3.73 (m, 8H), 3.16 (s, 3H), 3.12-3.03 (m, 3H), 2.84 (q, J = 7.9 Hz, 1H), 2.36-2.30 (m, 1H), 2.17-1.95 (m, 6H), 1.86-1.74 (m, 3H), 0.45 (t, J = 7.2 Hz, 3H). |
| | Example 46 | | |
| | 5-ethyl-6-fluoro-4-[3-methyl-9- | | |
| | (1,4-oxazepan-4-yl)-7- | | |
| | [[(2R,8S)-2-fluoro-1,2,3,5,6,7- | | |
| | hexahydropyrrolizin-8- | | |
| | yl]methoxy]pyrazolo[4,3- | | |
| | f]quinazolin-4-yl]naphthalen-2-ol | | |
| I-59 and 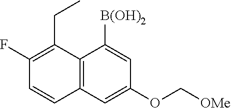 [8-ethyl-7-fluoro-3- (methoxymethoxy) naphthalen-1-yl]boronic acid | Example 47 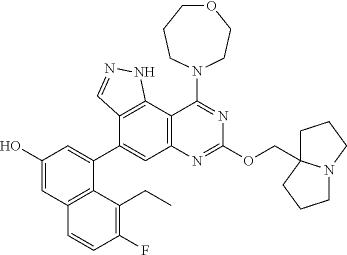 [SMILES: CCc1c(F)ccc2cc(O)cc(-c3cc4nc(OCC56CCCN5CCC6)nc(N5CCCOCC5)c4c4[nH]ncc34)c12] | 2.83 min, 597.4 [M + H]⁺ (Meth- od 1) | 13.33 (s, 1H), 9.89 (s, 1H), 8.42-8.32 (m, 1H), 7.77 (dd, J = 9.1, 6.1 Hz, 1H), 7.34 (t, J = 9.4 Hz, 1H), 7.30 (d, J = 2.5 Hz, 1H), 7.22 (t, J = 9.5 Hz, 1H), 7.03 (d, J = 2.6 Hz, 1H), 4.15- 3.99 (m, 2H), 3.99- 3.62 (m, 6H), 3.45- 3.19 (m, 4H), 3.01- 2.89 (m, 2H), 2.62- 2.24 (m, 2H), 2.14- 1.68 (m, 8H), 1.66- 1.50 (m, 2H), 0.65- 0.49 (m, 3H) |
| | Example 47 | | |
| | 5-ethyl-6-fluoro-4-[7- | | |
| | (1,2,3,5,6,7-hexahydropyrrolizin-8- | | |
| | ylmethoxy)-9-(1,4-oxazepan-4- | | |
| | yl)-1H-pyrazolo[3,4-f] | | |
| | quinazolin-4-yl]naphthalen-2-ol | | |
| I-57 and 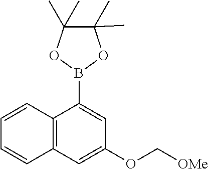 2-(3-(2-methoxymethoxy) naphthalene-1-yl)-4,4,5,5- tetramethyl-1,3,2-dioxaborolane | Example 48 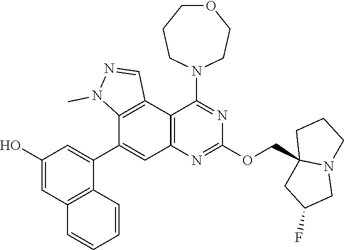 [SMILES: Cn1ncc2c3c(N4CCCOCC4)nc(OC[C@@]45CCCN4C[C@H](F)C5)nc3cc(-c3cc(O)cc4ccccc34)c21] | 3.07 min, 583.5 [M + H]⁺ (Meth- od 1) | 10.06 (s, 1H), 8.30 (s, 1H), 7.84 (d, J = 8.3 Hz, 1H), 7.48-7.43 (m, 1H), 7.41 (s, 1H), 7.33 (d, J = 1.8 Hz, 1H), 7.24-7.14 (m, 3H), 5.38-5.19 (m, 1H), 4.16-4.00 (m, 2H), 3.99-3.71 (m, 8H), 3.21-2.99 (m, 6H), 2.88-2.80 (m, 1H), 2.21-2.12 (m, 1H), 2.11-1.97 (m, 4H), 1.89-1.74 (m, 3H). |
| | Example 48 | | |
| | 4-[3-methyl-9-(1,4-oxazepan- | | |
| | 4-yl)-7-[[(2R,8S)-2-fluoro- | | |
| | 1,2,3,5,6,7- | | |
| | hexahydropyrrolizin-8- | | |
| | yl]methoxy]pyrazolo[4,3- | | |
| | f]quinazolin-4-yl]naphthalen-2-ol | | |
| I-60 and 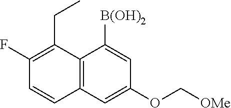 [SMILES: CCc1c(F)ccc2cc(OCOC)cc(B(O)O)c12] [8-ethyl-7-fluoro-3- (methoxymethoxy) naphthalen-1-yl]boronic acid | Example 49 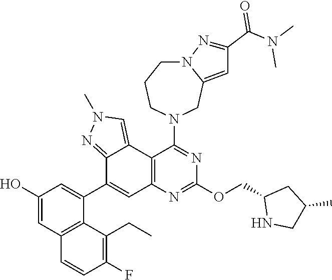 [SMILES: CCc1c(F)ccc2cc(O)cc(-c3cc4nc(OC[C@@H]5C[C@H](C)CN5)nc(N5CCCn6nc(C(=O)N(C)C)cc6C5)c4c4cn(C)nc34)c12] Example 49 | 2.71 min, 692.9 [M H]⁺ (Meth- od 1) | 9.77 (d, J = 5.0 Hz, 1H), 8.32 (s, 1H), 7.73 (dd, J = 9.1, 6.1 Hz, 1H), 7.31 (t, J = 9.4 Hz, 1H), 7.27 (d, J = 2.6 Hz, 1H), 7.12 (br s, 1H), 7.00-6.97 (m, 1H), 6.24 (s, 1H), 5.71 (br s, 1H), 4.92- 4.81 (m, 1H), 4.73 (d, J = 15.9 Hz, 1H), 4.57-4.44 (m, 2H), 4.2-3.92 (m, 6H), 3.88-3.75 (m, 1H), 3.73-3.62 (m, 1H), 3.61-3.52 (m, 1H), 3.29-3.20 (m, 3H), 2.97-2.81 (m, 4H), 2.45-2.35 (m, 1H), 2.56-2.00 (m, 5H), 1.65-1.37 (m, 1H), 1.05 (d, J = 6.4 Hz, 3H), 0.61-0.52 (m, 3H). |
| | 5-[4-(8-ethyl-7-fluoro-3- | | |
| | hydroxy-1-naphthyl)-2-methyl- | | |
| | 7-[[(2S,4S)-4-methylpyrrolidin- | | |
| | 2-yl]methoxy]pyrazolo[4,3- | | |
| | f]quinazolin-9-yl]-N,N-dimethyl- | | |
| | 4,6,7,8-tetrahydropyrazolo[1,5- | | |
| | a][1,4]diazepine-2-carboxamide | | |
| I-65 and 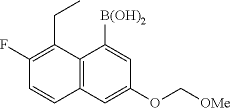 [8-ethyl-7-fluoro-3- (methoxymethoxy) naphthalen-1-yl]boronic acid | Example 59 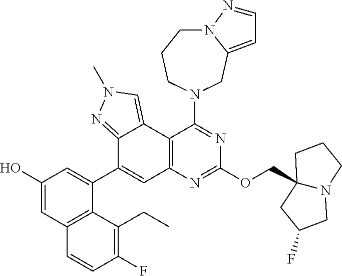 [SMILES: CCc1c(F)ccc2cc(O)cc(-c3cc4nc(OC[C@@]56CCCN5C[C@H](F)C6)nc(N5CCCn6nccc6C5)c4c4cn(C)nc34)c12] | 3.00 min, 665.4 [M + H]⁺ (Meth- od 1) | 9.80 (s, 1H), 8.47 (s, 1H), 7.75 (dd, J = 6.3, 8.9 Hz, 1H), 7.35-7.28 (m, 3H), 7.19 (d, J = 2.1 Hz, 1H), 6.99 (d, J = 2.9 Hz, 1H), 5.83 (s, 1H), 5.27 (d, J = 55.0 Hz, 1H), 4.96-4.75 (m, 2H), 4.50-4.47 (m, 2H), 4.07-3.81 (m, 7H), 3.12-3.00 (m, 3H), 2.85-2.79 (m, 1H), 2.24-1.75 (m, 10H), 0.55 (t, J = 7.0 Hz, 3H), |
| | Example 59 | | |
| | 5-ethyl-6-fluoro-4-[2-methyl-7- | | |
| | [[(2R,8S)-2-fluoro-1,2,3,5,6,7- | | |
| | hexahydropyrrolizin-8- | | |
| | yl]methoxy]-9-(4,6,7,8- | | |
| | tetrahydropyrazolo[1,5- | | |
| | a][1,4]diazepin-5- | | |
| | yl)pyrazolo[4,3-f]quinazolin-4- | | |
| | yl]naphthalen-2-ol | | |
| I-67 and 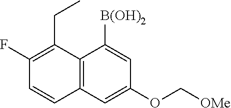 [8-ethyl-7-fluoro-3- (methoxymethoxy) naphthalen-1-yl]boronic acid | Example 72 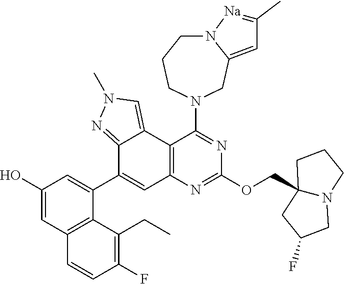 | 3.48 min, 679.3 [M + H]⁺ (Meth- od 6) | 9.81 (s, 1H), 8.47 (s, 1H), 7.75 (dd, J = 9.1, 6.0 Hz, 1H), 7.36-7.27 (m, 3H), 7.00 (d, J = 2.6 Hz, 1H), 5.62 (s, 1H), 5.28 (d, J = 54.3 Hz, 1H), 4.94-4.62 (m, 2H), 4.38 (d, J = 6.1 Hz, 2H), 4.07 (s, 3H), 4.06-3.93 (m, 2H), 3.87-3.76 (m, 1H), 3.09 (d, J = 8.3 Hz, 2H), 3.02 (s, 1H), 2.84 (t, J = 7.9 Hz, 1H), 2.47-2.05 (m, 1H), 2.03 (s, 3H), 2.02-1.68 (m, 1H), 0.62-0.46 (m, 3H) |
| | Example 72 | | |
| I-69 and 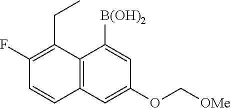 [8-ethyl-7-fluoro-3- (methoxymethoxy) naphthalen-1-yl]boronic acid | Example 73 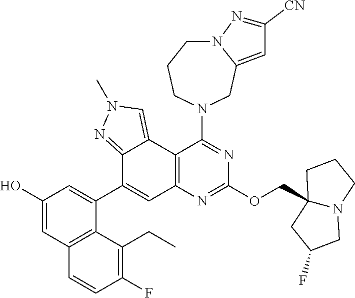 [SMILES: CCc1c(F)ccc2cc(O)cc(-c3cc4nc(OC[C@@]56CCCN5C[C@H](F)C6)nc(N5CCCn6nc(C#N)cc6C5)c4c4cn(C)nc34)c12] | 3.78 min, 690.3 [M + H]⁺ (Meth- od 6) | 9.81 (s, 1H), 8.50 (s, 1H), 7.75 (dd, J = 9.1, 6.1 Hz, 1H), 7.38-7.26 (m, 3H), 6.98 (d, J = 2.6 Hz, 1H), 6.78 (s, 1H), 5.38-5.17 (m, 1H), 4.87 (q, J = 16.1 Hz, 2H), 4.62-4.48 (m, 2H), 4.08 (s, 3H), 4.03-3.83 (m, 3H), 3.16-2.96 (m, 3H), 2.89-2.76 (m, 1H), 2.47-2.30 (m, 2H), 2.24-1.69 (m, 9H), 0.61-0.48 (m, 3H). |
| | Example 73 | | |
| | 5-[4-(8-ethyl-7-fluoro-3- | | |
| | hydroxy-1-naphthyl)-7- | | |
| | [[(2R,8S)-2-fluoro-1,2,3,5,6,7- | | |
| | hexahydropyrrolizin-8- | | |
| | yl]methoxy]-2-methyl- | | |
| | pyrazolo[4,3-f]quinazolin-9-yl]- | | |
| | 4,6,7,8-tetrahydropyrazolo[1,5- | | |
| | a][1,4]diazepine-2-carbonitrile | | |
| I-74 and 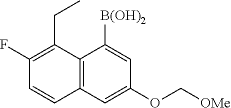 [8-ethyl-7-fluoro-3- (methoxymethoxy) naphthalen-1-yl]boronic acid | Example 78 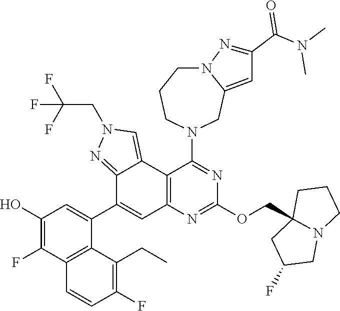 [SMILES: CCc1c(F)ccc2c(F)c(O)cc(-c3cc4nc(OC[C@@]56CCCN5C[C@H](F)C6)nc(N5CCCn6nc(C(=O)N(C)C)cc6C5)c4c4cn(CC(F)(F)F)nc34)c12] | 3.30 min, 804.6 [M + H]⁺ (Meth- od 1) | 9.84 (s, 1H), 8.64 (s, 1H), 7.74 (dd, J = 9.1, 6.1 Hz, 1H), 7.42 (s, 1H), 7.38-7.26 (m, 2H), 7.03 (d, J = 2.5 Hz, 1H), 6.30 (s, 1H), 5.55-5.40 (m, 2H), 5.40-5.19 (m, 1H), 4.96 (d, J = 15.8 Hz, 1H), 4.83 (d, J = 15.8 Hz, 1H), 4.60-4.45 (m, 2H), 4.17-3.92 (m, 3H), 3.91-3.78 (m, 1H), 3.29-2.77 (m, 10H), 2.43-2.33 (m, 1H), 2.28-1.94 (m, 6H), 1.94-1.71 (m, 3H), 0.52 (t, J = 7.2 Hz, 3H) |
| | Example 78 | | |
| | 5-[4-(8-ethyl-7-fluoro-3- | | |
| | hydroxy-1-naphthyl)-7- | | |
| | [[(2R,8S)-2-fluoro-1,2,3,5,6,7- | | |
| | hexahydropyrrolizin-8- | | |
| | yl]methoxy]-2-(2,2,2- | | |
| | trifluoroethyl)pyrazolo[4,3- | | |
| | f]quinazolin-9-yl]-N,N-dimethyl- | | |
| | 4,6,7,8-tetrahydropyrazolo[1,5- | | |
| | a][1,4]diazepine-2- | | |
| | carboxamide | | |
| I-77 and 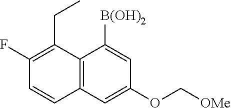 [8-ethyl-7-fluoro-3- (methoxymethoxy) naphthalen-1-yl]boronic acid | Example 79 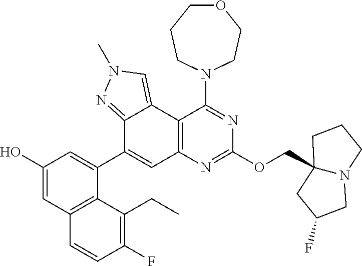 | 3.12 min, 643.6 [M + H]⁺ (Meth- od 1) | 9.77 (s, 1H), 8.44 (s, 1H), 7.75 (dd, J = 9.1, 6.1 Hz, 1H), 7.35-7.25 (m, 2H), 6.86 (d, J = 2.6 Hz, 1H), 5.40-5.19 (m, 1H), 4.22-4.12 (m, 1H), 4.11-4.00 (m, 4H), 3.91-3.70 (m, 8H), 3.18-2.98 (m, 3H), 2.90-2.78 (m, 1H), 2.35-2.12 (m, 6H), 2.12-1.91 (m, 4H), 1.91-1.72 (m, 3H), 0.38 (t, J = 7.3 Hz, 3H) |
| | Example 79 | | |
| | 4-[2,5-dimethyl-9-(1,4- | | |
| | oxazepan-4-yl)-7-[[(2R,8S)-2- | | |
| | fluoro-1,2,3,5,6,7- | | |
| | hexahydropyrrolizin-8- | | |
| | yl]methoxy]pyrazolo[4,3- | | |
| | f]quinazolin-4-yl]-5-ethyl-6- | | |
| | fluoro-naphthalen-2-ol | | |
| I-79 and 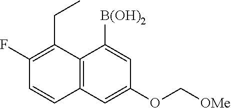 [8-ethyl-7-fluoro-3- (methoxymethoxy) naphthalen-1-yl]boronic acid | Example 80 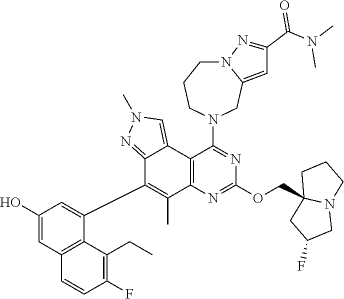 | 3.15 min, 642.4 [M + H]+ (Meth- od 1) | 9.78 (s, 1H), 8.43 (s, 1H), 7.76 (dd, J = 9.1, 6.1 Hz, 1H), 7.37-7.25 (m, 2H), 6.87 (d, J = 2.6 Hz, 1H), 6.19 (s, 1H), 5.38-5.15 (m, 1H), 4.92 (d, J = 16.0 Hz, 1H), 4.78 (d, J = 15.9 Hz, 1H), 4.61- 4.45 (m, 2H), 4.13- 3.93 (m, 6H), 3.91- 3.78 (m, 1H), 3.25 (s, 3H), 3.14-2.96 (m, 3H), 2.92 (s, 3H), 2.87-2.76 (m, 1H), 2.36-1.93 (m, 10H), 1.89-1.68 (m, 3H), 0.43 (t, J = 7.3 Hz, 3H) |
| | Example 80 | | |
| | 5-[4-(8-ethyl-7-fluoro-3- | | |
| | hydroxy-1-naphthyl)-2,5- | | |
| | dimethyl-7-[[(2R,8S)-2-fluoro- | | |
| | 1,2,3,5,6,7- | | |
| | hexahydropyrrolizin-8- | | |
| | yl]methoxy]pyrazolo[4,3- | | |
| | f]quinazolin-9-yl]-N,N-dimethyl- | | |
| | 4,6,7,8-tetrahydropyrazolo[1,5- | | |
| | a][1,4]diazepine-2- | | |
| | carboxamide | | |
| I-84 and 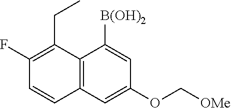 [8-ethyl-7-fluoro-3- (methoxymethoxy) naphthalen-1-yl]boronic acid | Example 87 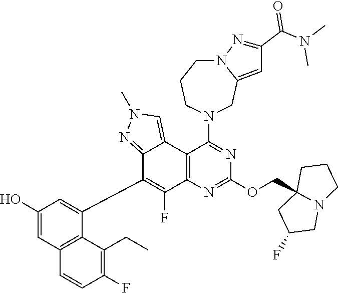 [SMILES: CCc1c(F)ccc2cc(O)cc(-c3c(F)c4nc(OC[C@@]56CCCN5C[C@H](F)C6)nc(N5CCCn6nc(C(=O)N(C)C)cc6C5)c4c4cn(C)nc34)c12] | 3.51 min, 754.4 [M + H]+ (Meth- od 6) | 9.87 (s, 1H), 8.53 (s, 1H), 7.78 (dd, J = 9.1, 6.1 Hz, 1H), 7.39-7.29 (m, 2H), 7.06-7.00 (m, 1H), 6.27 (s, 1H), 5.36- 5.15 (m, 1H), 4.95 (d, J = 16.0 Hz, 1H), 4.83 (d, J = 15.7 Hz, 1H), 4.58-4.44 (m, 2H), 4.13-3.85 (m, 7H), 3.25 (s, 3H), 3.11-2.96 (m, 3H), 2.93 (s, 3H), 2.86- 2.76 (m, 1H), 2.25- 1.90 (m, 7H), 1.87- 1.69 (m, 3H), 0.58 (t, J = 7.3 Hz, 3H) |
| | Example 87 | | |
| | 5-[4-(8-ethyl-7-fluoro-3- | | |
| | hydroxy-1-naphthyl)-5-fluoro-2- | | |
| | methyl-7-[[(2R,8S)-2-fluoro- | | |
| | 1,2,3,5,6,7- | | |
| | hexahydropyrrolizin-8- | | |
| | yl]methoxy]pyrazolo[4,3- | | |
| | f]quinazolin-9-yl]-N,N-dimethyl- | | |
| | 4,6,7,8-tetrahydropyrazolo[1,5- | | |
| | a][1,4]diazepine-2- | | |
| | carboxamide | | |
| I-86 and 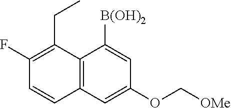 [8-ethyl-7-fluoro-3- (methoxymethoxy) naphthalen-1-yl]boronic acid | Example 88 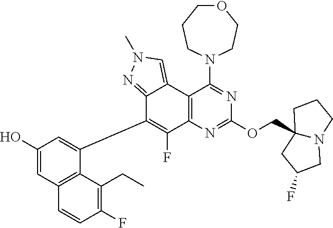 [SMILES: CCc1c(F)ccc2cc(O)cc(-c3c(F)c4nc(OC[C@@]56CCCN5C[C@H](F)C6)nc(N5CCCOCC5)c4c4cn(C)nc34)c12] | 3.60 min, 647.3 [M + H]+ (Meth- od 6) | 9.85 (s, 1H), 8.52 (s, 1H), 7.77 (dd, J = 9.1, 6.2 Hz, 1H), 7.38-7.29 (m, 2H), 7.00 (d, J = 2.6 Hz, 1H), 5.39-5.17 (m, 1H), 4.20-3.98 (m, 5H), 3.92-3.71 (m, 8H), 3.15-2.98 (m, 3H), 2.89-2.77 (m, 1H), 2.47-2.32 (m, 1H), 2.24-1.95 (m, 6H), 1.90-1.70 (m, 3H), 0.56 (t, J = 7.0 Hz, 3H). |
| | Example 88 | | |
| | 5-ethyl-6-fluoro-4-[5-fluoro-2- | | |
| | methyl-9-(1,4-oxazepan-4-yl)- | | |
| | 7-[[(2R,8S)-2-fluoro- | | |
| | 1,2,3,5,6,7-hexahydropyrrolizin-8- | | |
| | yl]methoxy]pyrazolo[4,3-f] | | |
| | quinazolin-4-yl]naphthalen-2-ol | | |
| I-92 and 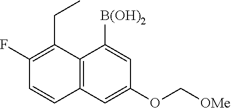 [8-ethyl-7-fluoro-3- (methoxymethoxy) naphthalen-1-yl]boronic acid | Example 91 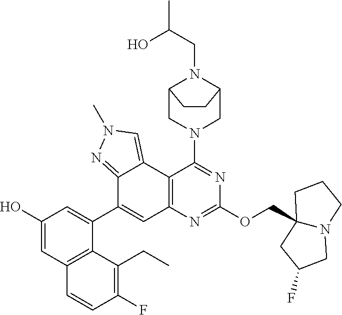 [SMILES: CCc1c(F)ccc2cc(O)cc(-c3cc4nc(OC[C@@]56CCCN5C[C@H](F)C6)nc(N5CC6CCC(C5)N6CC(C)O)c4c4cn(C)nc34)c12] | 3.23 min, 698.3 [M + H]+ (Meth- od 6) | 9.80 (s, 1H), 8.34 (s, 1H), 7.68-7.72 (m, 1H), 7.33-7.28 (m, 3H), 6.99-6.98 (m, 1H), 5.37-5.24 (d, J = 49.97, 1H), 4.54- 4.49 (m, 2H), 4.36- 4.20 (m, 1H), 4.21- 4.02 (m, 2H), 4.10 (s, 3H), 3.84-3.74 (m, 1H), 3.15-3.08 (m, 3H), 2.88-2.79 (m, 3H), 2.71-2.60 (m, 1H), 2.41-2.74 (m, 15H), 1.10-1.09 (d, J = 5.72, 3H), 0.53- 0.50 (t, J = 6.96, 3H). |
| | Example 91 | | |
| | 5-ethyl-6-fluoro-4-[7-[[(2R,8S)- | | |
| | 2-fluoro-1,2,3,5,6,7- | | |
| | hexahydropyrrolizin-8- | | |
| | yl]methoxy]-9-[3-(2- | | |
| | hydroxypropyl)-3,8- | | |
| | diazabicyclo[3.2.1]octan-8-yl]- | | |
| | 2-methyl-pyrazolo[4,3- | | |
| | f]quinazolin-4-yl]naphthalen-2-ol | | |
| I-94 and 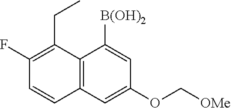 [8-ethyl-7-fluoro-3- (methoxymethoxy) naphthalen-1-yl]boronic acid | Example 92 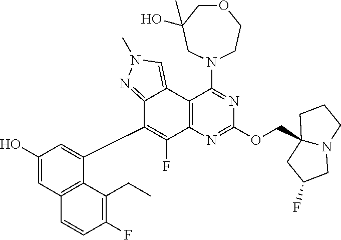 [SMILES: CCc1c(F)ccc2cc(O)cc(-c3c(F)c4nc(OC[C@@]56CCCN5C[C@H](F)C6)nc(N5CCOCC(C)(O)C5)c4c4cn(C)nc34)c12] | 3.49 min, 677.3 [M + H]+ (Meth- od 6) | 9.89-9.82 (m, 1H), 8.55-8.48 (m, 1H), 7.82-7.73 (m, 1H), 7.38-7.29 (m, 2H), 7.05-6.98 (m, 1H), 5.39-5.17 (m, 2H), 4.21-4.01 (m, 5H), 4.01-3.66 (m, 6H), 3.60-3.36 (m, 2H), 3.17-2.96 (m, 3H), 2.89-2.76 (m, 1H), 2.45-2.32 (m, 1H), 2.25-1.94 (m, 4H), 1.90-1.70 (m, 3H), 1.02 (s, 2H), 0.85 (s, 1H), 0.57 (t, J = 7.3 Hz, 3H) |
| | Example 92 | | |
| | 4-[4-(8-ethyl-7-fluoro-3- | | |
| | hydroxy-1-naphthyl)-5-fluoro-2- | | |
| | methyl-7-[[(2R,8S)-2-fluoro- | | |
| | 1,2,3,5,6,7- | | |
| | hexahydropyrrolizin-8- | | |
| | yl]methoxy]pyrazolo[4,3- | | |
| | f]quinazolin-9-yl]-6-methyl-1,4- | | |
| | oxazepan-6-ol | | |
| I-98 and 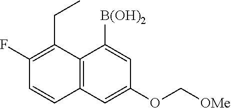 [8-ethyl-7-fluoro-3- (methoxymethoxy) naphthalen-1-yl]boronic acid | Example 95 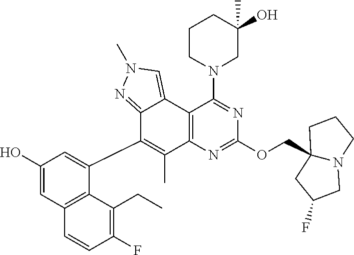 [SMILES: CCc1c(F)ccc2cc(O)cc(-c3c(C)c4nc(OC[C@@]56CCCN5C[C@H](F)C6)nc(N5CCC[C@@](C)(O)C5)c4c4cn(C)nc34)c12] | 3.84 min, 657.3 [M + H]+ (Meth- od 6) | 9.77 (s, 1H), 8.54 (s, 1H), 8.25 (s, 1H), 7.78-7.72 (m, 1H), 7.35-7.26 (m, 2H), 6.87-6.83 (m, 1H), 5.29 (d, J = 54.3 Hz, 1H), 4.66-4.60 (m, 1H), 4.20-4.13 (m, 1H), 4.08-4.02 (s, 4H), 2.29-2.23 (m, 1H), 2.21-2.13 (m, 6H), 2.10-2.01 (m, 5H), 1.87-1.70 (m, 6H), 1.69-1.56 (m, 3H), 1.25-1.21 (m, 1H), 1.13-1.11 (m, 2H), 1.07-0.99 (m, 1H), 0.45-0.30 (m, 3H) |
| | Example 95 | | |
| | (3R)-1-[4-(8-ethyl-7-fluoro-3- | | |
| | hydroxy-1-naphthyl)-2,5- | | |
| | dimethyl-7-[[(2R,8S)-2-fluoro- | | |
| | 1,2,3,5,6,7- | | |
| | hexahydropyrrolizin-8- | | |
| | yl]methoxy]pyrazolo[4,3-f] | | |
| | quinazolin-9-yl]-3-methyl- | | |
| | piperidin-3-ol | | |
| I-100 and 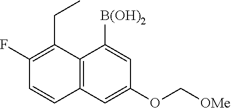 [SMILES: CCc1c(F)ccc2cc(OCOC)cc(B(O)O)c12] [8-ethyl-7-fluoro-3- (methoxymethoxy) naphthalen-1-yl]boronic acid | Example 96 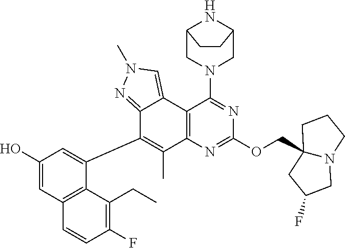 [SMILES: CCc1c(F)ccc2cc(O)cc(-c3c(C)c4nc(OC[C@@]56CCCN5C[C@H](F)C6)nc(N5CC6CCC(C5)N6)c4c4cn(C)nc34)c12] Example 96 | 3.41 min, 654.3 [M H]+ (Meth- od 6) | 9.78 (s, 1H), 8.30 (s, 1H), 7.77-7.73 (m, 1H), 7.32-7.27 (m, 2H), 6.85-6.84 (d, J = 2.60, 1H), 5.35-5.22 (d, J = 53.25, 1H), 4.19-4.15 (m, 2H), 4.05 (s, 3H), 3.84 (s, 3H), 3.65-3.62 (m, 1H), 3.55-3.51 (m, 1H), 3.11-3.09 (m, 2H), 3.02 (m, 1H), 2.87-2.81 (m, 1H), 2.19 (s, 3H), 2.17- 2.15 (m, 2H), 2.08 (m, 1H), 2.03-1.97 (m, 3H), 1.87-1.77 (m, 6H), 1.24 (s, 1H), 0.40-0.36 (t, J = 7.46, 3H). |
| | tert-butyl 3-[4-bromo-7- | | |
| | [[(2R,8S)-2-fluoro-1,2,3,5,6,7- | | |
| | hexahydropyrrolizin-8- | | |
| | yl]methoxy]-2,5-dimethyl- | | |
| | pyrazolo[4,3-f]quinazolin-9-yl]- | | |
| | 3,8-diazabicyclo[3.2.1]octane- | | |
| | 8-carboxylate | | |
| I-104 and 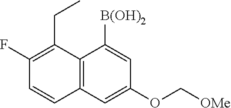 [8-ethyl-7-fluoro-3- (methoxymethoxy) naphthalen-1-yl]boronic acid | Example 99 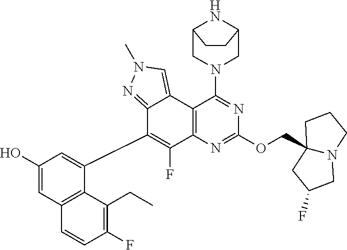 [SMILES: CCc1c(F)ccc2cc(O)cc(-c3c(F)c4nc(OC[C@@]56CCCN5C[C@H](F)C6)nc(N5CC6CCC(C5)N6)c4c4cn(C)nc34)c12] | 3.33 min, 658.3 [M + H]+ (Meth- od 6) | 9.85 (s, 1H), 8.39 (s, 1H), 7.77 (dd, J = 9.2, 6.1 Hz, 1H), 7.38-7.27 (m, 2H), 7.01 (d, J = 2.6 Hz, 1H), 5.39-5.18 (m, 1H), 4.19-3.98 (m, 6H), 3.94-3.81 (m, 1H), 3.63-3.43 (m, 4H), 3.18-2.97 (m, 3H), 2.90-2.78 (m, 1H), 2.46-2.33 (m, 1H), 2.23-1.95 (m, 4H), 1.90-1.66 (m, 4H), 1.64-1.51 (m, 3H), 0.55 (t, J = 7.3 Hz, 3H) Exchangeable proton not seen. |
| | Example 99 | | |
| | 4-[9-(3,8- | | |
| | diazabicyclo[3.2.1]octan-3-yl)- | | |
| | 5-fluoro-2-methyl-7-[[(2R,8S)- | | |
| | 2-fluoro-1,2,3,5,6,7- | | |
| | hexahydropyrrolizin-8- | | |
| | yl]methoxy]pyrazolo[4,3- | | |
| | f]quinazolin-4-yl]-5-ethyl-6- | | |
| | fluoro-naphthalen-2-ol | | |
| I-106 and 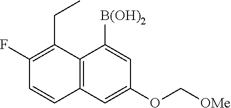 [8-ethyl-7-fluoro-3- (methoxymethoxy) naphthalen-1-yl]boronic acid | Example 100 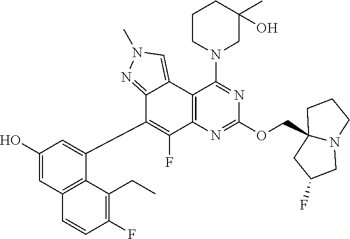 [SMILES: CCc1c(F)ccc2cc(O)cc(-c3c(F)c4nc(OC[C@@]56CCCN5C[C@H](F)C6)nc(N5CCCC(C)(O)C5)c4c4cn(C)nc34)c12] | 3.63 min, 661.3 [M + H]+ (Meth- od 6) | 9.90-9.83 (m, 1H), 8.73-8.53 (m, 1H), 7.77 (dd, J = 9.0, 6.1 Hz, 1H), 7.40-7.27 (m, 2H), 7.01 (d, J = 2.6 Hz, 1H), 5.41- 5.19 (m, 1H), 4.67- 4.52 (m, 1H), 4.24- 4.00 (m, 5H), 3.69- 3.40 (m, 2H), 3.39- 3.23 (m, 2H), 3.19- 2.98 (m, 3H), 2.91- 2.78 (m, 1H), 2.45- 2.34 (m, 1H), 2.24- 1.95 (m, 5H), 1.93- 1.57 (m, 6H), 1.09 (1.5H), 1.01 (s, 1.5H), 0.61-0.50 (m, 3H) |
| | Example 100 | | |
| | (3R)-1-[4-bromo-5-fluoro-2- | | |
| | methyl-7-[[(2R,8S)-2-fluoro- | | |
| | 1,2,3,5,6,7-hexahydropyrrolizin-8- | | |
| | yl]methoxy]pyrazolo[4,3-f] | | |
| | quinazolin-9-yl]-3-methyl- | | |
| | piperidin-3-ol | | |
| I-108 and 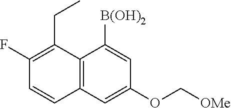 [8-ethyl-7-fluoro-3- (methoxymethoxy) naphthalen-1-yl]boronic acid | Example 102 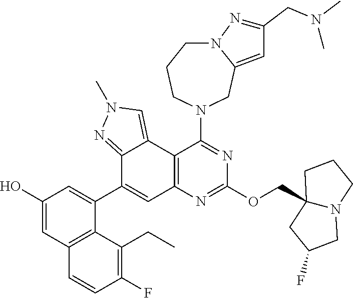 [SMILES: CCc1c(F)ccc2cc(O)cc(-c3cc4nc(OC[C@@]56CCCN5C[C@H](F)C6)nc(N5CCCn6nc(CN(C)C)cc6C5)c4c4cn(C)nc34)c12] Example 102 | 3.25 min, 722.4 [M + H]+ (Meth- od 6) | 8.09 (d, J = 10.7 Hz, 1H), 7.54-7.41 (m, 2H), 7.13 (t, J = 9.3 Hz, 1H), 7.10-7.01 (m, 1H), 6.02 (s, 1H), 5.28 (d, J = 53.4 Hz, 1H), 4.60 (q, J = 16.2 Hz, 2H), 4.49 (s, 2H), 4.28-4.11 (m, 2H), 4.08 (d, J = 3.3 Hz, 2H), 3.90 (s, 2H), 3.58-3.41 (m, 2H), 3.27 (d, J = 20.0 Hz, 1H), 3.23-3.12 (m, 1H), 2.98 (s, 1H), 2.47 (dd, J = 14.4, 7.5 Hz, 1H), 2.34 (d, J = 2.3 Hz, 5H), 2.32- 2.19 (m, 4H), 2.15 (d, J = 10.1 Hz, 1H), 2.01-1.89 (m, 1H), 1.85-1.50 (m 10H), 0.67-0.58 (m, 3H). |
| | 4-[9-[2- | | |
| | [(dimethylamino)methyl]- | | |
| | 4,6,7,8-tetrahydropyrazolo[1,5- | | |
| | a][1,4]diazepin-5-yl]-2-methyl- | | |
| | 7-[[(2R,8S)-2-fluoro- | | |
| | 1,2,3,5,6,7-hexahydropyrrolizin-8- | | |
| | yl]methoxy]pyrazolo[4,3- | | |
| | f]quinazolin-4-yl]-5-ethyl-6- | | |
| | fluoro-naphthalen-2-ol | | |
| I-119 and 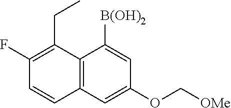 [SMILES: CCc1c(F)ccc2cc(OCOC)cc(B(O)O)c12] [8-ethyl-7-fluoro-3- (methoxymethoxy) naphthalen-1-yl] boronic acid | Example 109 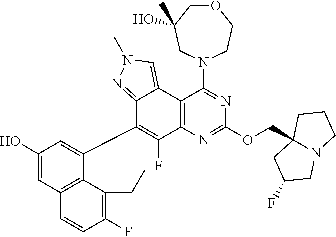 [SMILES: CCc1c(F)ccc2cc(O)cc(-c3c(F)c4nc(OC[C@@]56CCCN5C[C@H](F)C6)nc(N5CCOC[C@@](C)(O)C5)c4c4cn(C)nc34)c12] | 3.02 min, 677.7 [M H]+ (Meth- od 1) | 9.89-9.82 (m, 1H), 8.54-8.49 (m, 1H), 7.81-7.74 (m, 1H), 7.37-7.29 (m, 2H), 7.04-6.99 (m, 1H), 5.38-5.17 (m, 2H), 4.18-4.01 (m, 5H), 4.01-3.68 (m, 6H), 3.59-3.36 (m, 2H), 3.16-2.94 (m, 3H), 2.89-2.76 (m, 1H), 2.45-2.32 (m, 1H), 2.22-1.94 (m, 4H), 1.88-1.70 (m, 3H), 1.02 (s, 2H), 0.84 (s, 1H), 0.56 (t, J = 7.3 Hz, 3H). |
| | Example 109 | | |
| | (6S)-4-[4-(8-ethyl-7-fluoro-3- | | |
| | hydroxy-1-naphthyl)-5-fluoro-2- | | |
| | methyl-7-[[(2R,8S)-2-fluoro- | | |
| | 1,2,3,5,6,7-hexahydropyrrolizin-8- | | |
| | yl]methoxy]pyrazolo[4,3- | | |
| | f]quinazolin-9-yl]-6-methyl-1,4- | | |
| | oxazepan-6-ol | | |
| I-121 and 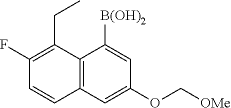 [8-ethyl-7-fluoro-3- (methoxymethoxy) naphthalen-1-yl]boronic acid | Example 110 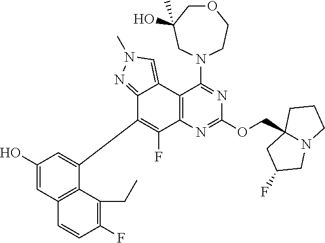 [SMILES: CCc1c(F)ccc2cc(O)cc(-c3c(F)c4nc(OC[C@@]56CCCN5C[C@H](F)C6)nc(N5CCOC[C@](C)(O)C5)c4c4cn(C)nc34)c12] | 3.01 min, 677.5 [M + H]+ (Meth- od 1) | 9.89-9.82 (m, 1H), 8.56-8.49 (m, 1H), 7.83-7.72 (m, 1H), 7.39-7.29 (m, 2H), 7.06-6.99 (m, 1H), 5.39-5.18 (m, 2H), 4.21-4.03 (m, 5H), 4.01-3.67 (m, 6H), 3.59-3.38 (m, 2H), 3.18-2.96 (m, 3H), 2.88-2.78 (m, 1H), 2.45-2.32 (m, 1H), 2.32-1.95 (m, 4H), 1.89-1.71 (m, 3H), 1.02 (s, 2H), 0.85 (s, 1H), 0.57 (t, J = 7.3 Hz, 3H). |
| | Example 110 | | |
| | (6R)-4-[4-(8-ethyl-7-fluoro-3- | | |
| | hydroxy-1-naphthyl)-5-fluoro-2- | | |
| | methyl-7-[[(2R,8S)-2-fluoro- | | |
| | 1,2,3,5,6,7- | | |
| | hexahydropyrrolizin-8- | | |
| | yl]methoxy]pyrazolo[4,3- | | |
| | f]quinazolin-9-yl]-6-methyl-1,4- | | |
| | oxazpan-6-ol | | |
| I-123 and 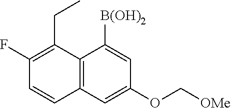 [8-ethyl-7-fluoro-3- (methoxymethoxy) naphthalen-1-yl]boronic acid | Example 111 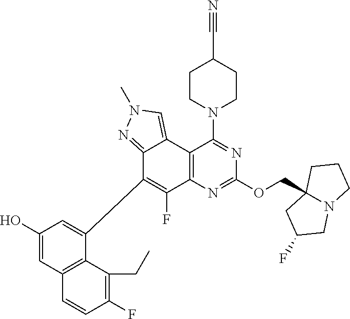 [SMILES: CCc1c(F)ccc2cc(O)cc(-c3c(F)c4nc(OC[C@@]56CCCN5C[C@H](F)C6)nc(N5CCC(C#N)CC5)c4c4cn(C)nc34)c12] | 3.18 min, 656.6 [M + H]+ (Meth- od 1) | 9.87 (s, 1H), 8.55 (s, 1H), 7.78 (dd, J = 9.1, 6.1 Hz, 1H), 7.39-7.29 (m, 2H), 7.00 (d, J = 2.6 Hz, 1H), 5.41-5.19 (m, 1H), 4.27-4.00 (m, 5H), 3.75-3.60 (m, 2H), 3.41-3.27 (m, 2H), 3.25-2.95 (m, 4H), 2.93-2.77 (m, 1H), 2.43-2.34 (m, 1H), 2.27-1.98 (m, 8H), 1.95-1.72 (m, 3H), 0.56 (t, J = 7.2 Hz, 3H). |
| | Example 111 | | |
| | 1-[4-(8-ethyl-7-fluoro-3- | | |
| | hydroxy-1-naphthyl)-5-fluoro-2- | | |
| | methyl-7-[[(2R,8S)-2-fluoro-1,2,3,5,6,7- | | |
| | hexahydropyrrolizin-8-yl]methoxy]pyrazolo[4,3- | | |
| | f]quinazolin-9-yl]piperidine-4-carbonitrile | | |
| 1-126 and 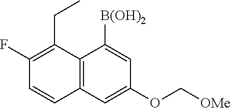 [8-ethyl-7-fluoro-3- (methoxymethoxy) naphthalen-1-yl]boronic acid | Example 112 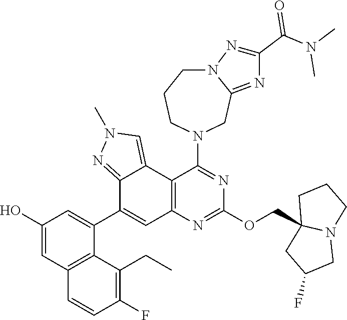 [SMILES: CCc1c(F)ccc2cc(O)cc(-c3cc4nc(OC[C@@]56CCCN5C[C@H](F)C6)nc(N5CCCn6nc(C(=O)N(C)C)nc6C5)c4c4cn(C)nc34)c12] | 2.94 min, 737.3 [M + H]+ (Meth- od 1) | 9.83 (1H, s), 8.57 (1H, s), 7.74 (1H, dd, J = 9.1, 6.1 Hz), 7.36 (1H, d, J = 0.5 Hz), 7.35-7.28 (2H, m), 7.00-6.97 (1H, m), 5.60-5.30 (1H, m), 5.04-4.91 (2H, m), 4.38-4.31 (2H, m), 4.27-3.98 (7H, m), 3.13 (3H, s), 2.99 (3H, s), 2.44- 1.84 (13H, m). |
| | Example 112 | | |
| | 8-[4-(8-ethyl-7-fluoro-3- | | |
| | hydroxy-1-naphthyl)-2-methyl- | | |
| | 7-[[(2R,8S)-2-fluoro- | | |
| | 1,2,3,5,6,7- | | |
| | hexahydropyrrolizin-8- | | |
| | yl]methoxy]pyrazolo[4,3- | | |
| | f]quinazolin-9-yl]-N,N-dimethyl- | | |
| | 5,6,7,9-tetrahydro-[1,2,4]triazolo[1,5-a] | | |
| | [1,4]diazepine-2-carboxamide | | |
| I-128 and 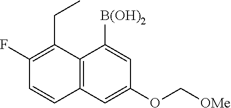 [8-ethyl-7-fluoro-3- (methoxymethoxy) naphthalen-1-yl]boronic acid | Example 113 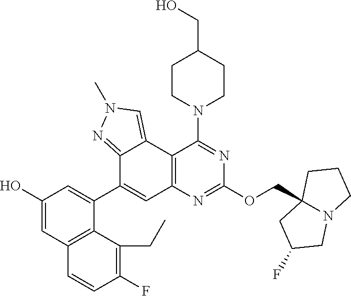 [SMILES: CCc1c(F)ccc2cc(O)cc(-c3cc4nc(OC[C@@]56CCCN5C[C@H](F)C6)nc(N5CCC(CO)CC5)c4c4cn(C)nc34)c12] | 2.88 min, 643.5 [M + H]+ (Meth- od 1) | 9.79 (s, 1H), 8.40 (s, 1H), 7.74 (dd, J = 6.1, 9.2 Hz, 1H), 7.36 (s, 1H), 7.31 (t, J = 9.4 Hz, 1H), 7.28 (d, J = 2.6 Hz, 1H), 6.97 (d, J = 2.6 Hz, 1H), 5.28 (dbr, J = 53.5 Hz, 1H), 4.55 (t, J = 10.4 Hz, 1H), 4.13 (dd, J = 5.6, 10.4 Hz, 1H), 4.10 (s, 3H), 4.04 (dd, J = 2.8, 10.4 Hz, 1H), 3.90 (mc, 1H), 3.44- 3.35 (m, 2H), 3.14- 2.78 (m, 5H), 2.43- 2.34 (m, 1H), 2.21- 1.62 (m, 12H), 1.60- 1.40 (m, 2H), 0.53 (t, J = 7.2 Hz, 3H). |
| | Example 113 | | |
| | 5-ethyl-6-fluoro-4-[9-[4- | | |
| | (hydroxymethyl)-1-piperidyl]-2- | | |
| | methyl-7-[[(2R,8S)-2-fluoro- | | |
| | 1,2,3,5,6,7- | | |
| | hexahydropyrrolizin-8- | | |
| | yl]methoxy]pyrazolo[4,3- | | |
| | f]quinazolin-4-yl]naphthalen-2-ol | | |
| I-130 and 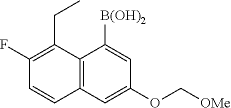 [8-ethyl-7-fluoro-3- (methoxymethoxy) naphthalen-1-yl]boronic acid | Example 114 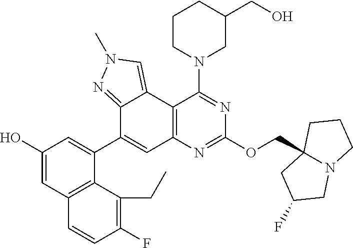 [SMILES: CCc1c(F)ccc2cc(O)cc(-c3cc4nc(OC[C@@]56CCCN5C[C@H](F)C6)nc(N5CCCC(CO)C5)c4c4cn(C)nc34)c12] Example 114 | 2.94 min, 643.5 [M + H]+ (Meth- od 1) | 9.79 (s, 1H), 8.41 (s, 1H), 7.74 (dd, J = 6.1, 9.2 Hz, 1H), 7.36 (d, J = 2.4 Hz, 1H), 7.35-7.28 (m, 1H), 7.28 (d, J = 2.6 Hz, 1H), 6.99-6.96 (m, 1H), 5.32 (d, J = 53.7 Hz, 1H), 4.60-4.51 (m, 1H), 4.25-4.12 (m, 1H), 4.09 (s, 3H), 4.01-3.91 (m, 1H), 3.88-3.75 (m, 1H), 3.46-3.34 (m, 2H), 3.14-2.78 (m, 5H), 2.43-2.34 (m, 1H), 2.21-1.62 (m, 12H), 1.30-1.20 (m, 2H), 0.52 (t, J = 7.3 Hz, 3H). |
| | 5-ethyl-6-fluoro-4-[9-[3- | | |
| | (hydroxymethyl)-1-piperidyl]-2- | | |
| | methyl-7-[[(2R,8S)-2-fluoro- | | |
| | 1,2,3,5,6,7- | | |
| | hexahydropyrrolizin-8- | | |
| | yl]methoxy]pyrazolo[4,3- | | |
| | f]quinazolin-4-yl]naphthalen-2-ol | | |
| I-132 and 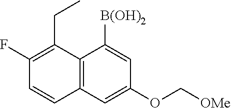 [8-ethyl-7-fluoro-3- (methoxymethoxy) naphthalen-1-yl]boronic acid | Example 115 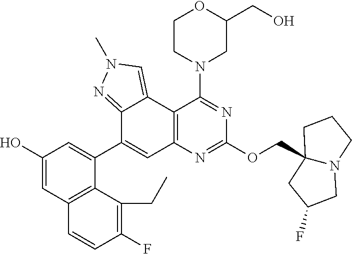 [SMILES: CCc1c(F)ccc2cc(O)cc(-c3cc4nc(OC[C@@]56CCCN5C[C@H](F)C6)nc(N5CCOC(CO)C5)c4c4cn(C)nc34)c12] Example 115 5-ethyl-6-fluoro-4-[9-[2-(hydroxymethyl)morpholin-4- yl]-2-methyl-7-[[(2R,8S)-2-fluoro-1,2,3,5,6,7- hexahydropyrrolizin-8-yl]methoxy]pyrazolo[4,3-f] quinazolin-4-yl]naphthalen-2-ol | 2.88 min, 645.5 [M + H]+ (Meth- od 1) | 9.80 (d, J = 1.8 Hz, 1H), 8.46 (d, J = 4.3 Hz, 1H), 7.75 (dd, J = 6.0, 9.1 Hz, 1H), 7.38 (d, J = 1.7 Hz, 1H), 7.31 (t, J = 9.0 Hz, 1H), 7.28 (t, J = 2.6 Hz, 1H), 6.97 (t, J = 2.5 Hz, 1H), 5.29 (dbr, J = 54.1 Hz, 1H), 4.82 (t, J = 5.6 Hz, 1H), 4.19-4.02 (m, 2H), 4.09 (sbr, 3H), 4.02-3.90 (m, 2H), 3.90-3.78 (m, 2H), 3.78-3.68 (m, 1H), 3.20-2.75 (m, 6H), 2.40-2.33 (m, 1H), 2.22-1.70 (m, 8H), 0.57-0.48 (m, 3H). |
| I-134 and 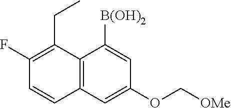 [8-ethyl-7-fluoro-3- (methoxymethoxy) naphthalen-1-yl]boronic acid | Example 116 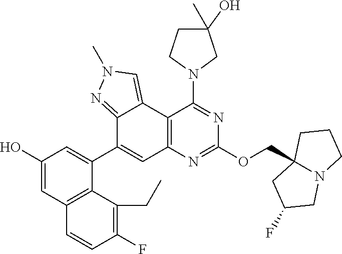 [SMILES: CCc1c(F)ccc2cc(O)cc(-c3cc4nc(OC[C@@]56CCCN5C[C@H](F)C6)nc(N5CCC(C)(O)C5)c4c4cn(C)nc34)c12] | 2.67 min, 629.5 [M + H]+ (Meth- od 1) | 9.80-9.77 (m, 1H), 8.44-8.37 (m, 1H), 7.77-7.71 (m, 1H), 7.35-7.25 (m, 3H), 7.00-6.95 (m, 1H), 5.39-5.18 (m, 1H), 4.84-4.77 (m, 1H), 4.27-4.18 (m, 0.5H), 4.15-3.93 (m, 5H), 3.88-3.80 (m, 0.5H), 3.69-3.61 (m, 0.5H), 3.61-3.55 (m, 0.5H), 3.46-3.36 (m, 1H), 3.14-3.00 (m, 3H), 2.88-2.79 (m, 1H), 2.40-2.35 (m, 1H), 2.19-1.73 (m, 10H), 1.41-1.31 (m, 3H), 0.58-0.48 (m, 3H). |
| | Example 116 | | |
| | 1-[4-(8-ethyl-7-fluoro-3- | | |
| | hydroxy-1-naphthyl)-2-methyl- | | |
| | 7-[[(2R,8S)-2-fluoro-1,2,3,5,6,7- | | |
| | hexahydropyrrolizin-8- | | |
| | yl]methoxy]pyrazolo[4,3- | | |
| | f]quinazolin-9-yl]-3-methyl- | | |
| | pyrrolidin-3-ol | | |
| I-136 and 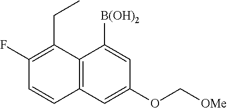 [8-ethyl-7-fluoro-3- (methoxymethoxy) naphthalen-1-yl]boronic acid | Example 117 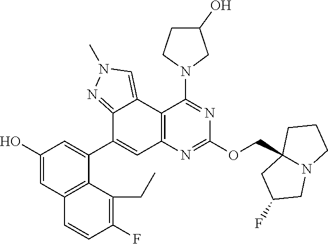 [SMILES: CCc1c(F)ccc2cc(O)cc(-c3cc4nc(OC[C@@]56CCCN5C[C@H](F)C6)nc(N5CCC(O)C5)c4c4cn(C)nc34)c12] | 2.61 min, 615.4 [M + H]+ (Meth- od 1) | 9.81 (m, 1H), 8.46- 8.37 (m, 1H), 7.78- 7.70 (m, 1H), 7.35- 7.25 (m, 3H), 7.01- 6.95 (m, 1H), 5.40- 5.19 (m, 1H), 5.07- 4.94 (m, 1H), 4.46- 4.35 (m, 1H), 4.17- 3.98 (m, 6H), 3.89- 3.62 (m, 1H), 3.46- 3.37 (m, 1H), 3.16- 2.99 (m, 3H), 2.90- 2.79 (m, 1H), 2.42- 2.37 (m, 1H), 2.20- 1.71 (m, 10H), 0.58- 0.46 (m, 3H). |
| | Example 117 | | |
| | 1-[4-(8-ethyl-7-fluoro-3- | | |
| | hydroxy-1-naphthyl)-2-methyl- | | |
| | 7-[[rac-(2R,8S)-2-fluoro- | | |
| | 1,2,3,5,6,7-hexahydropyrrolizin-8- | | |
| | yl]methoxy]pyrazolo[4,3- | | |
| | f]quinazolin-9-yl]pyrrolidin-3-ol | | |
| I-156 and 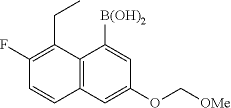 [8-ethyl-7-fluoro-3- (methoxymethoxy) naphthalen-1-yl]boronic acid | Example 180 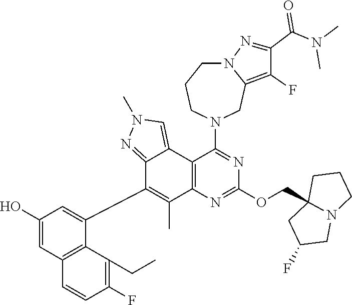 [SMILES: CCc1c(F)ccc2cc(O)cc(-c3c(C)c4nc(OC[C@@]56CCCN5C[C@H](F)C6)nc(N5CCCn6nc(C(=O)N(C)C)c(F)c6C5)c4c4cn(C)nc34)c12] | 3.17 min, 769.0 [M + H]+ (Meth- od 1) | 9.77 (s, 1H), 8.48 (s, 1H), 7.75 (dd, J = 6.1, 9.1 Hz, 1H), 7.34-7.27 (m, 2H), 6.86 (d, J = 2.6 Hz, 1H), 5.28 (dbr, J = 55.0 Hz, 1H), 4.86 (dbr, J = 15.6 Hz, 1H), 4.74 (dbr, J = 15.7 Hz, 1H), 4.56- 4.40 (m, 2H), 4.14- 3.82 (m, 4H), 4.06 (s, 3H), 3.11 (s, 3H), 3.06-2.95 (m, 2H), 2.94 (s, 3H), 2.88- 2.75 (m, 1H), 2.31- 1.92 (m, 8H), 2.19 (s, 3H), 1.88-1.70 (m, 3H), 0.41 (t, J = 7.3 Hz, 3H). |
| | Example 180 | | |
| | 5-[4-(8-ethyl-7-fluoro-3- | | |
| | hydroxy-1-naphthyl)-2,5- | | |
| | dimethyl-7-[[(2R,8S)-2-fluoro- | | |
| | 1,2,3,5,6,7-hexahydropyrrolizin-8- | | |
| | yl]methoxy]pyrazolo[4,3- | | |
| | f]quinazolin-9-yl]-3-fluoro-N,N- | | |
| | dimethyl-4,6,7,8- | | |
| | tetrahydropyrazolo[1,5- | | |
| | a][1,4]diazepine-2-carboxamide | | |
| I-158 and 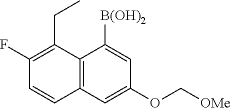 [8-ethyl-7-fluoro-3- (methoxymethoxy) naphthalen-1-yl]boronic acid | Example 181 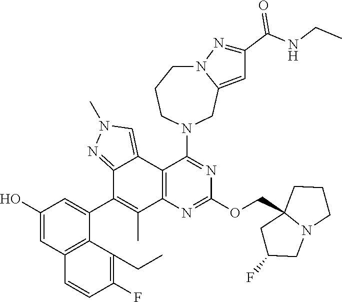 [SMILES: CCNC(=O)c1cc2n(n1)CCCN(c1nc(OC[C@@]34CCCN3C[C@H](F)C4)nc3c(C)c(-c4cc(O)cc5ccc(F)c(CC)c45)c4nn(C)cc4c13)C2] Example 181 | 3.16 min, 750.6 [M + H]+ (Metho 1) | 9.81 (s, 1H), 8.44 (s, 1H), 8.02-7.99 (m, 1H), 7.78-7.74 (m, 1H), 7.33-7.28 (m, 2H), 6.87-6.86 (m, 1H), 6.22 (s, 1H), 5.32 (s, 1H), 5.18 (s, 1H), 4.94-4.90 (m, 1H), 4.80-4.76 (m, 1H), 4.54-4.50 (m, 2H), 4.05 (s, 3H), 3.99-3.95 (m, 1H), 3.84 (s, 1H), 3.06- 3.04 (m, 3H), 2.98 (s, 1H), 2.80 (s, 2H), 2.30 (s, 1H), 2.19 (s, 3H), 2.02-1.97 (m, 4H), 1.83-1.74 (m, 3H), 1.24-1.23 (s, 3H), 1.16-1.12 (m, 1H), 1.06-1.02 (t, J = 13.08 Hz, 3H), 0.45- 0.41 (t, J = 7.44 Hz, 2H). |
| | N-ethyl-5-[4-(8-ethyl-7-fluoro- | | |
| | 3-hydroxy-1-naphthyl)-7- | | |
| | [[(2R,8S)-2-fluoro-1,2,3,5,6,7- | | |
| | hexahydropyrrolizin-8- | | |
| | yl]methoxy]-2,5-dimethyl- | | |
| | pyrazolo[4,3-f]quinazolin-9-yl]- | | |
| | 4,6,7,8-tetrahydropyrazolo[1,5- | | |
| | a][1,4]diazepine-2- | | |
| | carboxamide | | |
| I-165 and 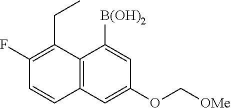 [8-ethyl-7-fluoro-3- (methoxymethoxy) naphthalen-1-yl]boronic acid | Example 186 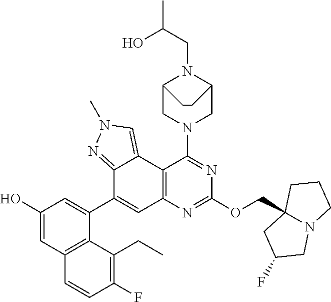 [SMILES: CCc1c(F)ccc2cc(O)cc(-c3cc4nc(OC[C@@]56CCCN5C[C@H](F)C6)nc(N5CC6CCC(C5)N6CC(C)O)c4c4cn(C)nc34)c12] | 3.27 min, 698.1 [M + H]+ (Meth- od 6) | 10.75 (s, 1H), 9.83 (d, J = 27.9 Hz, 3H), 8.28 (s, 1H), 7.76 (dd, J = 9.2, 6.1 Hz, 1H), 7.45-7.20 (m, 2H), 6.96 (d, J = 2.8 Hz, 1H), 5.67 (s, 1H), 5.54 (s, 1H), 4.59 (s, 3H), 4.23 (d, J = 55.4 Hz, 9H), 4.10 (s, 3H), 3.92-3.74 (m, 3H), 3.08 (s, 1H), 2.91 (d, J = 19.0 Hz, 2H), 2.70-2.65 (m, 2H), 1.33-1.14 (m, 5H), 0.88 (t, J = 7.1 Hz, 1H), 0.54 (t, J = 7.3 Hz, 4H). |
| | Example 186 | | |
| | 5-ethyl-6-fluoro-4-[7-[[(2R,8S)- | | |
| | 2-fluoro-1,2,3,5,6,7- | | |
| | hexahydropyrrolizin-8- | | |
| | yl]methoxy]-9-[8-(2- | | |
| | hydroxypropyl)-3,8- | | |
| | diazabicyclo[3.2.1]octan-3-yl]- | | |
| | 2-methyl-pyrazolo[4,3- | | |
| | f]quinazolin-4-yl]naphthalen-2-ol | | |

### Example 2, 5-ethynyl-6-fluoro-4-[2-methyl-9-(1,4-oxazepan-4-yl)-7-[[(2R,8S)-2-fluoro-1,2,3,5,6,7-hexahydropyrrolizin-8-yl]methoxy]pyrazolo[4,3-f]quinazolin-4-yl]naphthalen-2-ol

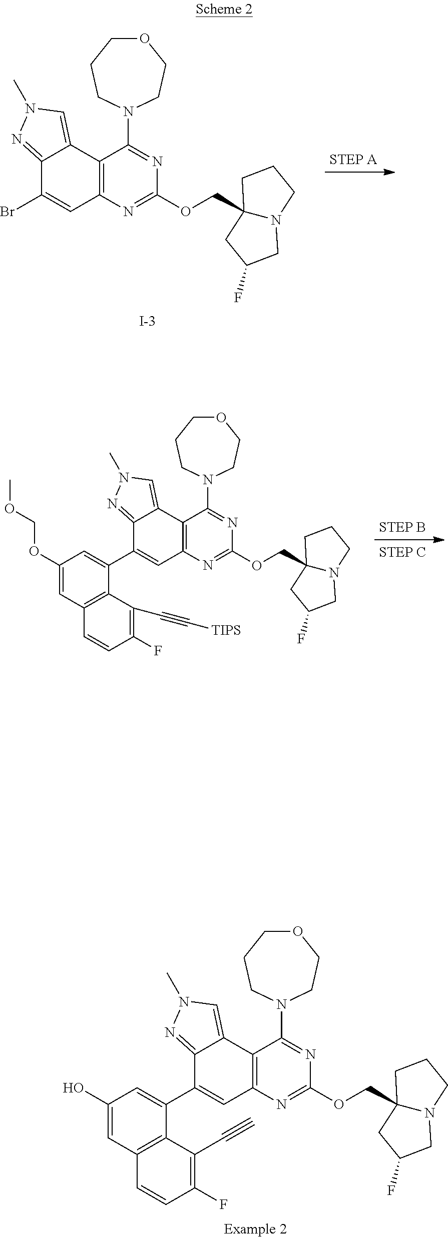

Step A, 2-[2-fluoro-6-(methoxymethoxy)-8-[2-methyl-9-(1,4-oxazepan-4-yl)-7-[[(2R,8S)-2-fluoro-1,2,3,5,6,7-hexahydropyrrolizin-8-yl]methoxy]pyrazolo[4,3-f]quinazolin-4-yl]-1-naphthyl]ethynyl-triisopropyl-silane. A mixture of 4-[4-bromo-2-methyl-7-[2R,8S)-2-fluoro-1,2,3,5,6,7-hexahydropyrrolizin-8-yl]methoxy]pyrazolo[4,3-f]quinazolin-9-yl]-1,4-oxazepane (1-3) (100 mg, 0.19 mmol) cataCXium A Pd G3 (28.04 mg, 0.04 mmol), Cs₂CO₃ (188.2 mg, 0.58 mmol) and [7-fluoro-3-(methoxymethoxy)-8-(2-triisopropylsilylethynyl)-1-naphthyl]boronic acid (165.7 mg, 0.39 mmol) in toluene (1.6 mL) and water (0.4 mL) was heated at 80° C. for 2 h under an atmosphere of N₂. After cooling to ambient temperature, the mixture was filtered through celite and the filtrate was concentrated under vacuum. The crude product was purified by silica gel chromatography (eluting with 50% EtOAc in pet. Ether) to afford 2-[2-fluoro-6-(methoxymethoxy)-8-[2-methyl-9-(1,4-oxazepan-4-yl)-7-[(2R,8S)-2-fluoro-1,2,3,5,6,7-hexahydropyrrolizin-8-yl]methoxy]pyrazolo[4,3-f]quinazolin-4-yl]-1-naphthyl]ethynyl-triisopropyl-silane (95 mg, 0.10 mmol, 54% yield).

LC-MS (ES⁺ Method 3): 3.983 min, m/z, 825.60 [M+H]⁺.

Step B, 4-[4-[8-ethynyl-7-fluoro-3-(methoxymethoxy)-1-naphthyl]-2-methyl-7-[[(2R,8S)-2-fluoro-1,2,3,5,6,7-hexahydropyrrolizin-8-yl]methoxy]pyrazolo[4,3-f]quinazolin-9-yl]-1,4-oxazepane. A solution of 2-[2-fluoro-6-(methoxymethoxy)-8-[2-methyl-9-(1,4-oxazepan-4-yl)-7-[[(2R,8S)-2-fluoro-1,2,3,5,6,7-hexahydropyrrolizin-8-yl]methoxy]pyrazolo[4,3-f]quinazolin-4-yl]-1-naphthyl]ethynyl-triisopropyl-silane (90 mg, 0.11 mmol) and potassium fluoride (19.01 mg, 0.33 mmol) in DMF (3 mL) was stirred at 25° C. for 1 hour. The mixture was diluted with water (5 mL) and extracted with EtOAc (20 mL×3). The combined organic layers were washed with brine (20 mL), dried over Na₂SO₄ and concentrated under vacuum and purified by silica gel column (pet.ether/EtOAc, 10/1 to 1/1) to give 4-[4-[8-ethynyl-7-fluoro-3-(methoxymethoxy)-1-naphthyl]-2-methyl-7-[[(2R,8S)-2-fluoro-1,2,3,5,6,7-hexahydropyrrolizin-8-yl]methoxy]pyrazolo[4,3-f]quinazolin-9-yl]-1,4-oxazepane (80 mg, 0.095 mmol, 88% yield)

LC-MS (ES⁺ Method 4): 1.30 min, m/z 669.30 [M+H]⁺.

Step C, 5-ethynyl-6-fluoro-4-[2-methyl-9-(1,4-oxazepan-4-yl)-7-[(2R,8S)-2-fluoro-1,2,3,5,6,7-hexahydropyrrolizin-8-yl]methoxy]pyrazolo[4,3-f]quinazolin-4-yl]naphthalen-2-ol. A mixture of 4-[4-[8-ethynyl-7-fluoro-3-(methoxymethoxy)-1-naphthyl]-2-methyl-7-[[(2R,8S)-2-fluoro-1,2,3,5,6,7-hexahydropyrrolizin-8-yl]methoxy]pyrazolo[4,3-f]quinazolin-9-yl]-1,4-oxazepane (45 mg, 0.07 mmol) and 4N HCl in dioxane (1 mL) in DCM (1 mL) and methanol (0.5 mL) was stirred at room temperature for 2 hours. The pH of the resulting mixture was adjusted to 8 with saturated Na₂CO₃ solution, extracted with EtOAc (15 mL×3), dried over anhydrous Na₂SO₄ and concentrated under vacuum to afford 5-ethynyl-6-fluoro-4-[2-methyl-9-(1,4-oxazepan-4-yl)-7-[[(2R,8S)-2-fluoro-1,2,3,5,6,7-hexahydropyrrolizin-8-yl]methoxy]pyrazolo[4,3-f]quinazolin-4-yl]naphthalen-2-ol (example 2) (8.2 mg, 0.013 mmol, 20% yield) as a white solid.

UPLC-MS (ES⁺, Method 3): 2.77 min, m/z 313.45 [M+H]²⁺.

¹H NMR (400 MHz, DMSO-d6) δ/ppm: 8.39 (d, J=7.2 Hz, 1H), 7.98 (dd, J=9.2, 6.0 Hz, 1H), 7.43-7.35 (m, 2H), 7.30-7.21 (m, 1H), 7.12 (d, J=2.5 Hz, 1H), 5.28 (d, J=54.3 Hz, 1H), 4.52 (s, 1H), 4.37 (d, J=1.9 Hz, 1H), 4.14-4.08 (m, 1H), 4.05 (s, 4H), 3.84 (dd, J=12.0, 5.4 Hz, 3H), 3.80-3.73 (m, 4H), 3.10 (dd, J=10.9, 4.0 Hz, 2H), 3.02 (s, 1H), 2.84 (t, J=7.4 Hz, 1H), 2.21-1.75 (m, 8H).

The following examples in table 18 were made by analogy with example 2 (scheme 2) starting from the intermediate and building blocks for step A as described in the table. The catalyst cataCXium A Pd G3 could be substituted for Pd(dtbpf)Cl₂ in step A. In examples where the naphthol building block does not contain a protecting group such as a methoxymethyl group (for example building block [7-fluoro-3-hydroxy-8-(2-triisopropylsilylethynyl)-1-naphthyl]boronic acid in the preparation of example 93), step C is not required.

TABLE 18

| | | UPLC-MS | |
|---|---|---|---|
| Step A, intermediate + | | (ES⁺): min, | ¹H NMR (400 MHz, |
| building block | Example Structure/Name | m/z | DMSO-d₆) δ/ppm |
| 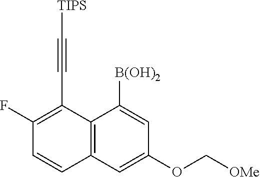 | 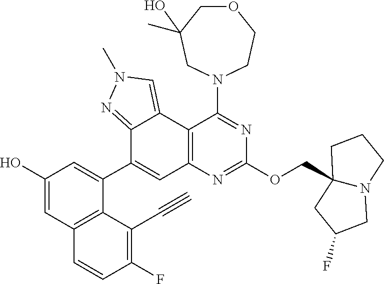 [SMILES: C#Cc1c(F)ccc2cc(O)cc(-c3cc4nc(OC[C@@]56CCCN5C[C@H](F)C6)nc(N5CCOCC(C)(O)C5)c4c4cn(C)nc34)c12] | 2.65 min, 655.5 [M + H]⁺ (Method 3) | 10.04 (m, 1H), 8.41- 8.39 (m, 1H), 7.97-7.92 (m, 1H), 7.44-7.39 (m, 1H), 7.36- 7.34 (m, 1H), 7.29- 7.23 (m, 1H), 7.16-7.14 (m, 1H), 6.09-5.17 (m, 2H), 4.16- 3.92 (m, 8H), 3.82- 3.72 (m, 2H), 3.63-3.55 (m, 2H), 3.48-3.39 (m, 2H), 3.12- 3.03 (m, 3H), 2.87- 2.83 (m, 1H), 2.16-2.02 (m, |
| | | | 3H), 1.87-1.81 |
| Intermediate I-8, | Example 11 | | (m, 3H), 1.12- |
| and | 4-[4-(8-ethynyl-7-fluoro-3- | | 1.05 (m, 3H). |
| [7-fluoro-3- | hydroxy-1-naphthyl)-2-methyl- | | |
| (methoxymethoxy)-8- | 7-[[(2R,8S)-2-fluoro- | | |
| (2-triisopropyl- | 1,2,3,5,6,7- | | |
| silylethynyl)- | hexahydropyrrolizin-8- | | |
| 1-naphthyl]boronic | yl]methoxy]pyrazolo[4,3- | | |
| acid | f]quinazolin-9-yl]-6-methyl-1,4- | | |
| | oxazepan-6-ol | | |
| 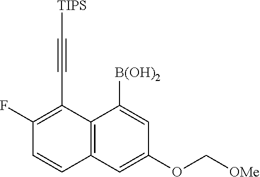 | 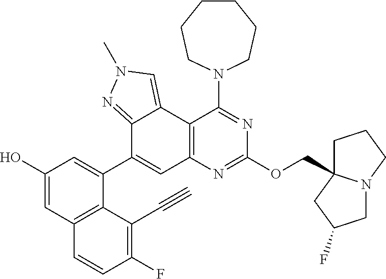 [SMILES: C#Cc1c(F)ccc2cc(O)cc(-c3cc4nc(OC[C@@]56CCCN5C[C@H](F)C6)nc(N5CCCCCC5)c4c4cn(C)nc34)c12] | 1.18 min, 623.3 [M + H]⁺ (Method 4) | 10.02 (s, 1H), 8.36 (s, 1H), 7.95 (dd, J = 9.2, 6.0 Hz, 1H), 7.42 (t, J = 9.0 Hz, 1H), 7.34 (d, J = 2.6 Hz, 1H), 7.22 (s, 1H), 7.13 (d, J = 2.5 Hz, 1H), 5.31 (d, J = 54.0 Hz, 1H), 4.13 (d, J = 7.4 Hz, 1H), 4.05 (s, 4H), 3.80-3.65 (m, 4H), 3.40 (s, 1H), 3.13 (m, 3H), 2.88 (s, 1H), 2.20- 2.05 (m, 3H), 1.86- 1.63 (m, 11H). |
| Intermediate I-10, | Example 12 | | |
| and | 4-[9-(azepan-1-yl)-2-methyl-7- | | |
| [7-fluoro-3- | [[(2R,8S)-2-fluoro-1,2,3,5,6,7- | | |
| (methoxymethoxy)-8- | hexahydropyrrolizin-8- | | |
| (2- | yl]methoxy]pyrazolo[4,3- | | |
| triisopropylsilylethynyl)- | f]quinazolin-4-yl]-5-ethynyl-6- | | |
| 1-naphthyl]boronic | fluoro-naphthalen-2-ol | | |
| acid | | | |
| 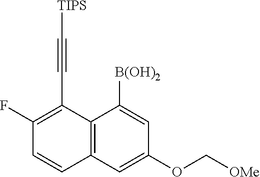 | 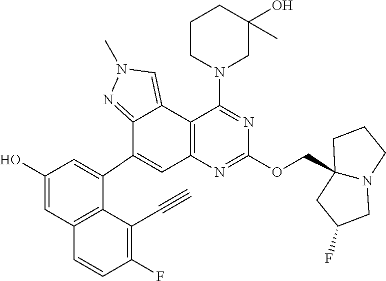 [SMILES: C#Cc1c(F)ccc2cc(O)cc(-c3cc4nc(OC[C@@]56CCCN5C[C@H](F)C6)nc(N5CCCC(C)(O)C5)c4c4cn(C)nc34)c12] | 0.67 min, 639.3 [M + H]⁺ (Method 4) | 9.65 (s, 1H), 8.54 (d, J = 48.5 Hz, 2H), 8.32 (d, J = 14.4 Hz, 1H), 8.10- 7.98 (m, 1H), 7.62 (t, J = 9.0 Hz, 1H), 7.48 (d, J = 2.3 Hz, 1H), 6.13 (t, J = 2.6 Hz, 1H), 6.07-5.93 (m, 1H), 5.30 (d, J = 54.3 Hz, 1H),4.50 (s, 3H), 4.22-4.08 (m, 2H), 3.86- 3.61 (m, 3H), 3.20- 2.99 (m, 4H), 2.92-2.75 (m, 1H), 2.25-1.95 (m, 4H), 1.91- |
| | | | 1.50 (m, 6H), 1.01 |
| Intermediate I-6, | Example 33 | | (d, J = 23.3 Hz, |
| and | 1-[4-(8-ethynyl-7-fluoro-3- | | 3H). |
| [7-fluoro-3- | hydroxy-1-naphthyl)-2-methyl- | | |
| (methoxymethoxy)-8- | 7-[[(2R,8S)-2-fluoro- | | |
| (2- | 1,2,3,5,6,7- | | |
| triisopropylsilylethynyl)- | hexahydropyrrolizin-8- | | |
| 1-naphthyl]boronic | yl]methoxy]pyrazolo[4,3- | | |
| acid | f]quinazolin-9-yl]-3-methyl- | | |
| | piperidin-3-ol | | |
| 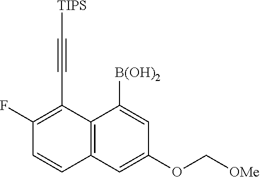 | 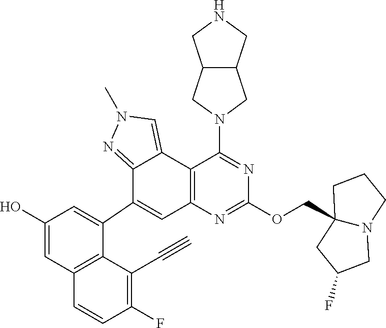 [SMILES: C#Cc1c(F)ccc2cc(O)cc(-c3cc4nc(OC[C@@]56CCCN5C[C@H](F)C6)nc(N5CC6CNCC6C5)c4c4cn(C)nc34)c12] | 3.10 min, m/z, 636.5 [M + H]⁺ (Method 4) | 8.43 (s, 1H), 7.93 (dd, J = 9.2, 5.8 Hz, 1H), 7.43-7.34 (m, 2H), 7.23- 7.16 (m, 2H), 5.28 (d, J = 53.6 Hz, 1H), 4.12-4.05 (m, 4H), 4.01- 3.97 (m, 1H), 3.75- 3.68 (m, 1H), 3.20-3.06 (m, 3H), 3.46 (s, 1H), 3.08- 2.78 (m, 10H), 2.17- 1.92 (m, 4H), 1.90- 1.76 (m, 3H). Exchangeable proton not seen. |
| Intermediate I-34, | Example 34 | | |
| and | 4-[9-(2,3,3a,4,6,6a-hexahydro- | | |
| [7-fluoro-3- | 1H-pyrrolo[3,4-c]pyrrol-5-yl)-2- | | |
| (methoxymethoxy)-8- | methyl-7-[[(2R,8S)-2-fluoro- | | |
| (2- | 1,2,3,5,6,7- | | |
| triisopropylsilylethynyl)- | hexahydropyrrolizin-8- | | |
| 1-naphthyl]boronic | yl]methoxy]pyrazolo[4,3- | | |
| acid | f]quinazolin-4-yl]-5-ethynyl-6- | | |
| | fluoro-naphthalen-2-ol | | |
| 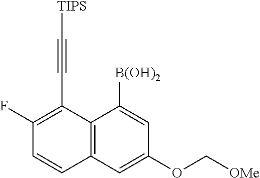 | 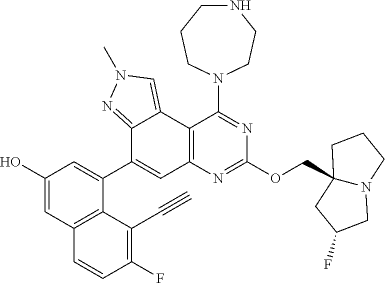 [SMILES: C#Cc1c(F)ccc2cc(O)cc(-c3cc4nc(OC[C@@]56CCCN5C[C@H](F)C6)nc(N5CCCNCC5)c4c4cn(C)nc34)c12] | 3.13 min, 624.5 [M + H]⁺ (Method 4) | 10.44 (s, 1H), 9.94 (s, 1H), 8.64 (s, 1H), 8.26 (d, J = 2.4 Hz, 1H), 8.12 (dd, J = 9.3, 6.1 Hz, 1H), 7.68 (t, J = 9.0 Hz, 1H), 7.53 (d, J = 2.3 Hz, 1H), 6.16 (t, J = 2.6 Hz, 1H), 5.99 (d, J = 2.7 Hz, 1H), 5.44-5.31 (m, 1H), 4.53 (s, 3H), 4.22-4.21 (m, 2H), 4.12-4.06 (m, 1H), 3.98- 3.92 (m, 1H), 3.67- 3.51 (m, 4H), 3.20-3.17 (m, |
| | | | 4H), 2.94 (s, 1H), |
| Intermediate I-24, | Example 35-Atropisomer 1 | | 2.21-2.16 (m, 3H) |
| and | 4-[9-(1,4-diazepan-1-yl)-2- | | 2.03-1.88 (m, |
| [7-fluoro-3- | methyl-7-[[(2R,8S)-2-fluoro- | | 6H). |
| (methoxymethoxy)-8- | 1,2,3,5,6,7- | | |
| (2- | hexahydropyrrolizin-8- | | |
| triisopropylsilylethynyl)- | yl]methoxy]pyrazolo[4,3- | | |
| 1-naphthyl]boronic | f]quinazolin-4-yl]-5-ethynyl-6- | | |
| acid | fluoro-naphthalen-2-ol | | |
| 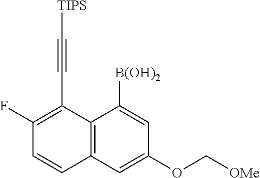 | 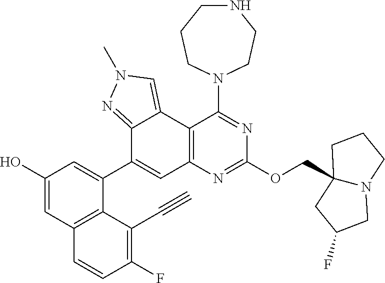 [SMILES: C#Cc1c(F)ccc2cc(O)cc(-c3cc4nc(OC[C@@]56CCCN5C[C@H](F)C6)nc(N5CCCNCC5)c4c4cn(C)nc34)c12] | | 10.82 (s, 1H), 10.09 (s, 1H), 8.48 (d, J = 1.3 Hz, 1H), 7.97 (dd, J = 9.2, 6.0 Hz, 1H), 7.43 (t, J = 9.0 Hz, 1H), 7.36 (d, J = 2.6 Hz, 1H), 7.30 (d, J = 1.3 Hz, 1H), 7.13 (d, J = 2.5 Hz, 1H), 5.65-5.52 (m, 1H), 4.56 (s, 2H), 4.07-4.02 (m, 4H), 3.92-3.78 (m, 5H), 3.68- 3.62 (m, 1H), 3.56- 3.55 (m, 1H), 3.49-3.45 (m, |
| | | | 3H), 3.28 (s, 1H), |
| Intermediate I-24, | Example 36-Atropisomer 2 | | 2.39-1.87 (m, |
| and | Example 35 | | 9H). |
| [7-fluoro-3- | 4-[9-(1,4-diazepan-1-yl)-2- | | |
| (methoxymethoxy)-8- | methyl-7-[[(2R,8S)-2-fluoro- | | |
| (2- | 1,2,3,5,6,7- | | |
| triisopropylsilylethynyl)- | hexahydropyrrolizin-8- | | |
| 1-naphthyl]boronic | yl]methoxy]pyrazolo[4,3- | | |
| acid | f]quinazolin-4-yl]-5-ethynyl-6- | | |
| | fluoro-naphthalen-2-ol | | |
| 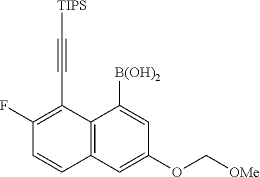 | 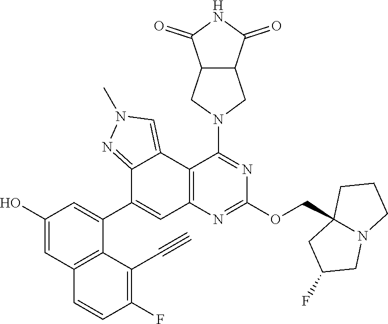 [SMILES: C#Cc1c(F)ccc2cc(O)cc(-c3cc4nc(OC[C@@]56CCCN5C[C@H](F)C6)nc(N5CC6C(=O)NC(=O)C6C5)c4c4cn(C)nc34)c12] | 0.83 min, m/z, 664.2 [M + H]⁺ (Method 4) | 9.56 (s, 1H), 8.60 (s, 1H), 8.42 (s, 1H), 8.31 (d, J = 2.4 Hz, 1H), 8.10 (dd, J = 9.2, 6.0 Hz, 1H), 7.66 (t, J = 8.9 Hz, 1H), 7.51 (d, J = 2.3 Hz, 1H), 6.11 (d, J = 41.8 Hz, 1H), 5.38 (s, 1H), 4.63 (d, J = 10.3 Hz, 1H), 4.49 (s, 3H), 4.12 (dd, J = 8.2, 5.3 Hz, 2H), 4.01 (s, 2H), 3.55- 3.53 (m, 4H), 3.11 (d, J = 11.7 Hz, 2H), 3.06-3.02 (m, 1H), 2.16- 1.97 (m, 4H), 1.90- 1.79 (m, 3H). |
| Intermediate I-20, | Example 37 | | |
| and | 5-[4-(8-ethynyl-7-fluoro-3- | | |
| [7-fluoro-3- | hydroxy-1-naphthyl)-2-methyl- | | |
| (methoxymethoxy)-8- | 7-[(2R,8S)-2-fluoro- | | |
| (2- | 1,2,3,5,6,7- | | |
| triisopropylsilylethynyl)- | hexahydropyrrolizin-8- | | |
| 1-naphthyl]boronic | yl]methoxy]pyrazolo[4,3- | | |
| acid | f]quinazolin-9-yl]-3a,4,6,6a- | | |
| | tetrahydropyrrolo[3,4-c]pyrrole- | | |
| | 1,3-dione | | |
| 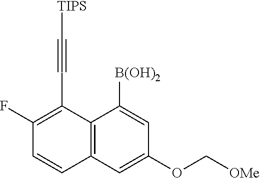 | 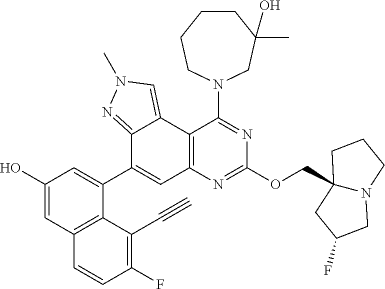 [SMILES: C#Cc1c(F)ccc2cc(O)cc(-c3cc4nc(OC[C@@]56CCCN5C[C@H](F)C6)nc(N5CCCCC(C)(O)C5)c4c4cn(C)nc34)c12] | 2.88 min 653.5 [M + H]⁺ (Method 4) | 10.05 (s, 1H), 8.32 (d, J = 21.5 Hz, 1H), 7.95 (m, 1H), 7.45-7.34 (m, 2H), 7.25 (d, J = 26.9 Hz, 1H), 7.20- 7.12 (m, 1H), 5.32 (d, J = 54.1 Hz, 1H), 4.66 (d, J = 10.3 Hz, 1H), 4.19-3.96 (m, 7H), 3.93-3.53 (m, 3H), 3.44 (d, J = 14.5 Hz, 1H), 3.17 (s, 2H), 2.89 (s, 1H), 2.28-1.97 (m, 3H), 1.98- 1.72 (m, 6H), 1.67- 1.57 (m, 2H), |
| | | | 1.47 (s, 1H), 1.01 |
| Intermediate I-12 and | Example 43 | | (d, J = 20.3 Hz, |
| [7-fluoro-3- | | | 3H). |
| (methoxymethoxy)-8- | | | |
| (2- | | | |
| triisopropylsilylethynyl)- | | | |
| 1-naphthyl]boronic | | | |
| acid | | | |
| 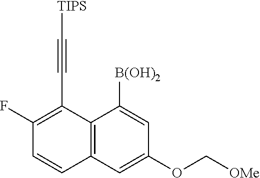 | 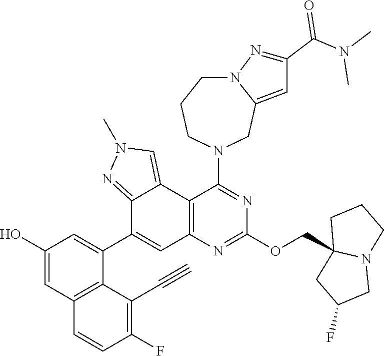 [SMILES: C#Cc1c(F)ccc2cc(O)cc(-c3cc4nc(OC[C@@]56CCCN5C[C@H](F)C6)nc(N5CCCn6nc(C(=O)N(C)C)cc6C5)c4c4cn(C)nc34)c12] | 2.77 min 732.9 [M + H]⁺ (Method 1) | 10.03 (s, 1H), 8.38 (s, 1H), 7.96 (dd, J = 9.2, 6.0 Hz, 1H), 7.43 (t, J = 9.0 Hz, 1H), 7.35 (d, J = 2.5 Hz, 1H), 7.26 (s, 1H), 7.14 (d, J = 2.5 Hz, 1H), 6.43 (s, 1H), 5.27 (d, J = 54.6 Hz, 1H), 4.82 (s, 2H), 4.55-4.43 (m, 2H), 4.06 (s, 3H), 4.04-3.84 (m, 4H), 3.49 (s, 1H), 3.29 (s, 3H), 3.14- 2.98 (s, 3H), 2.95 (s, 3H), 2.86-2.78 (m, 1H), 2.24-1.93 (m, 5H), 1.87-1.70 (m, 3H). |
| Intermediate I-28 and | Example 65 | | |
| [7-fluoro-3- | 5-[4-(8-ethynyl-7-fluoro-3- | | |
| (methoxymethoxy)-8- | hydroxy-1-naphthyl)-2-methyl- | | |
| (2- | 7-[[(2R,8S)-2-fluoro- | | |
| triisopropylsilylethynyl)- | 1,2,3,5,6,7- | | |
| 1-naphthyl]boronic | hexahydropyrrolizin-8- | | |
| acid | yl]methoxy]pyrazolo[4,3- | | |
| | f]quinazolin-9-yl]-N,N-dimethyl- | | |
| | 4,6,7,8-tetrahydropyrazolo[1,5- | | |
| | a][1,4]diazepine-2- | | |
| | carboxamide | | |
| 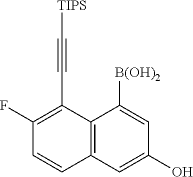 | 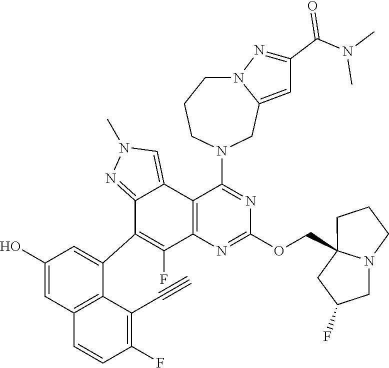 [SMILES: C#Cc1c(F)ccc2cc(O)cc(-c3c(F)c4nc(OC[C@@]56CCCN5C[C@H](F)C6)nc(N5CCCn6nc(C(=O)N(C)C)cc6C5)c4c4cn(C)nc34)c12] | 3.30 min 750.3 [M + H]⁺ (Method 6) | 10.09 (s, 1H), 8.44 (s, 1H), 7.98 (dd, J = 9.3, 6.0 Hz, 1H), 7.45 (t, J = 9.0 Hz, 1H), 7.40 (d, J = 2.5 Hz, 1H), 7.18 (d, J = 2.5 Hz, 1H), 6.44 (s, 1H), 5.38- 5.17 (m, 1H), 4.92- 4.79 (m, 2H), 4.54- 4.44 (m, 2H), 4.10- 3.89 (m, 7H), 3.58- 3.55 (m, 1H), 3.29 (m, 3H), 3.14-2.96 (m, 3H), 2.95 (s, 3H), 2.88-2.77 (m, 1H), 2.19-1.94 (m, 5H), 1.89-1.70 (m, 3H) |
| Intermediate I-84 and | Example 93 | | |
| [7-fluoro-3-hydroxy-8- | 5-[4-(8-ethynyl-7-fluoro-3- | | |
| (2- | hydroxy-1-naphthyl)-5-fluoro-2- | | |
| triisopropylsilylethynyl)- | methyl-7-[[(2R,8S)-2-fluoro- | | |
| 1-naphthyl]boronic | 1,2,3,5,6,7- | | |
| acid | hexahydropyrrolizin-8- | | |
| | yl]methoxy]pyrazolo[4,3- | | |
| | f]quinazolin-9-yl]-N,N-dimethyl- | | |
| | 4,6,7,8-tetrahydropyrazolo[1,5- | | |
| | a][1,4]diazepine-2- | | |
| | carboxamide | | |
| 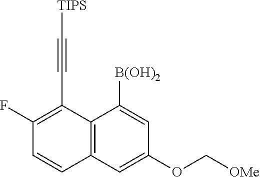 | 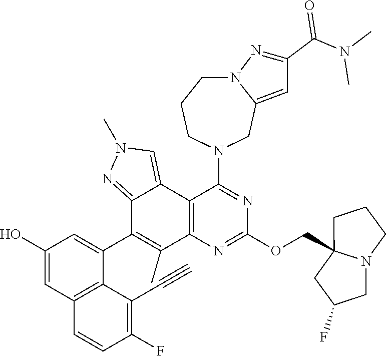 [SMILES: C#Cc1c(F)ccc2cc(O)cc(-c3c(C)c4nc(OC[C@@]56CCCN5C[C@H](F)C6)nc(N5CCCn6nc(C(=O)N(C)C)cc6C5)c4c4cn(C)nc34)c12] | 3.45 min 746.1 [M + H]⁺ (Method 6) | 10.00 (s, 1H), 8.32 (s, 1H), 8.23 (s, 1H), 7.96 (dd, J = 9.1, 5.9 Hz, 1H), 7.42 (t, J = 9.0 Hz, 1H), 7.34 (d, J = 2.6 Hz, 1H), 6.99 (d, J = 2.6 Hz, 1H), 6.54 (s, 1H), 6.39 (s, 1H), 5.36-5.32 (m, 1H), 5.22-5.17 (m, 1H), 4.81 (s, 3H), 4.50 (s, 4H), 4.11- 3.96 (m, 5H), 3.88 (s, 3H), 2.94 (s, 4H), 2.16 (s, 3H), 2.06 (d, J = 21.5 Hz, 1H), 1.77 (s, 4H), 1.27-1.21 (m, 2H) |
| Intermediate I-102 and | Example 97 | | |
| [7-fluoro-3- | 5-[4-(8-ethynyl-7-fluoro-3- | | |
| (methoxymethoxy)-8- | hydroxy-1-naphthyl)-2,5- | | |
| (2- | dimethyl-7-[[(2R,8S)-2-fluoro- | | |
| triisopropylsilylethynyl)- | 1,2,3,5,6,7- | | |
| 1-naphthyl]boronic | hexahydropyrrolizin-8- | | |
| acid | yl]methoxy]pyrazolo[4,3- | | |
| | f]quinazolin-9-yl]-N,N-dimethyl- | | |
| | 4,6,7,8-tetrahydropyrazolo[1,5- | | |
| | a][1,4]diazepine-2- | | |
| | carboxamide | | |
| 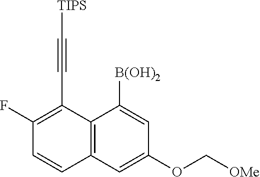 | 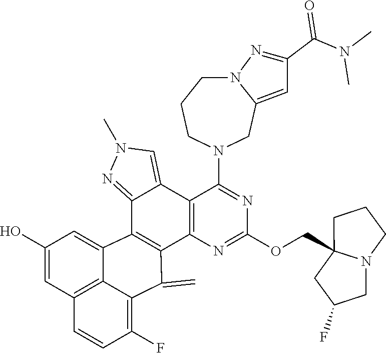 | 3.52 min 748.1 [M + H]⁺ (Method 6) | 9.84 (1H, s), 8.40 (1H, s), 8.14 (1H, s), 7.9 (1H, s), 7.85-7.80 (1H, m), 7.34 (1H, t, J = 9.84 Hz), 7.29-7.28 (1H, m), 6.94-6.91 (1H, m), 6.28-6.26 (1H, m), 5.68-5.60 (1H, m), 5.35-5.30 (1H, m), 5.18 (1H, s), 4.94-4.85 (1H, m), 4.82-4.76 (1H, m), 4.54-4.50 (2H, m), 4.49-4.44(1H, m), 4.07-4.04 (4H, m), 3.99-3.93 (3H, m), 3.92-3.81 (1H, m), 2.88 (3H, s), 2.73 (2H, s), 2.02 (6H, s), 2.00-1.93 (5H, m), 1.85-1.78 (m, 1 |
| | | | H), 0.87-0.78 (m, |
| Intermediate I-102 and | Example 98 | | 1H). |
| [7-fluoro-3- | 5-[4-(7-fluoro-3-hydroxy-8- | | |
| (methoxymethoxy)-8- | vinyl-1-naphthyl)-2,5-dimethyl- | | |
| (2- | 7-[[(2R,8S)-2-fluoro- | | |
| triisopropylsilylethynyl)- | 1,2,3,5,6,7- | | |
| 1-naphthyl]boronic | hexahydropyrrolizin-8- | | |
| acid | yl]methoxy]pyrazolo[4,3- | | |
| | f]quinazolin-9-yl]-N,N-dimethyl- | | |
| | 4,6,7,8-tetrahydropyrazolo[1,5- | | |
| | a][1,4]diazepine-2- | | |
| | carboxamide | | |
| 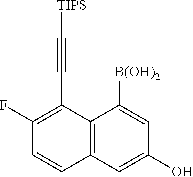 | 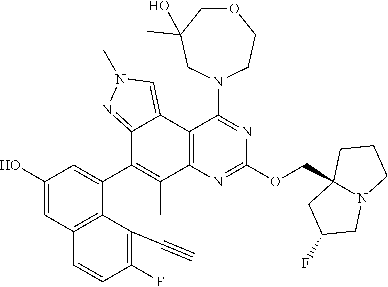 [SMILES: C#Cc1c(F)ccc2cc(O)cc(-c3c(C)c4nc(OC[C@@]56CCCN5C[C@H](F)C6)nc(N5CCOCC(C)(O)C5)c4c4cn(C)nc34)c12] | 2.94 min 669.5 [M + H]⁺ (Method 1) | 10.01(s, 1H), 8.35(s, 1H), 7.97- 7.94(q, 1H), 7.43- 7.38(t, J = 8.96, 1H), 7.34(d, J = 2.56, 1H), 7.00-6.99(m, 1H), 6.17(br, 1H), 5.35-5.21(d, J = 56.33, 1H), 4.19- 4.14(m, 1H), 4.08- 4.06(m, 1H) 4.02(s, 3H), 3.90- 3.87(m, 1H), 3.83- 3.80(m, 2H), 3.73- 3.69(m, 2H), 3.61- 3.55(m, 2H), 3.10- 3.07(m, 2H), 3.01(s, 1H) 2.83(br, 1H), |
| | | | 2.19(s, 3H), 2.09- |
| Intermediate I-115 and | Example 185 | | 1.99(m, 3H), 1.86- |
| [7-fluoro-3-hydroxy-8- | 4-[4-(8-ethynyl-7-fluoro-3- | | 1.78(m, 4H), |
| (2- | hydroxy-1-naphthyl)-7- | | 1.23(s, 1H), 1.13(s, |
| triisopropylsilylethynyl)- | [[(2R,8S)-2-fluoro-1,2,3,5,6,7- | | 3H), |
| 1-naphthyl]boronic | hexahydropyrrolizin-8- | | |
| acid | yl]methoxy]-2,5-dimethyl- | | |
| | pyrazolo[4,3-f]quinazolin-9-yl]- | | |
| | 6-methyl-1,4-oxazepan-6-ol | | |

### Example 3, 2-amino-4-[2-methyl-9-(1,4-oxazepan-4-yl)-7-[[(2R,8S)-2-fluoro-1,2,3,5,6,7-hexahydropyrrolizin-8-yl]methoxy]pyrazolo[4,3-f]quinazolin-4-yl]benzothiophene-3-carbonitrile

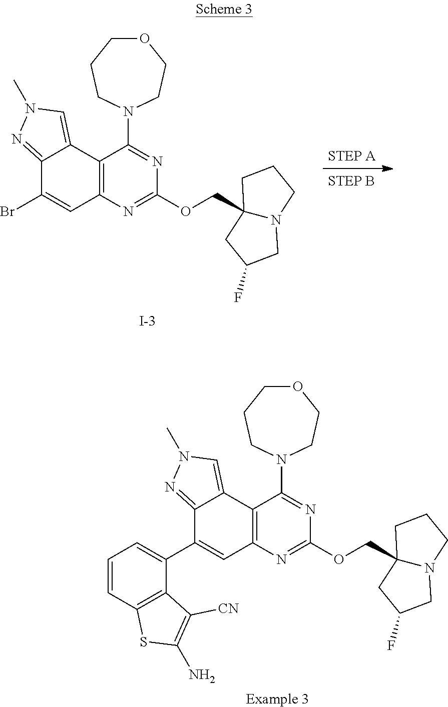

Step A, tert-butyl N-[3-cyano-4-[2-methyl-9-(1,4-oxazepan-4-yl)-7-[[(2R,8S)-2-fluoro-1,2,3,5,6,7-hexahydropyrrolizin-8-yl]methoxy]pyrazolo[4,3-f]quinazolin-4-yl]benzothiophen-2-yl]carbamate. A mixture of 4-(4-bromo-7-(((2R,7aS)-2-fluorotetrahydro-1H-pyrrolizin-7a (5H)-yl) methoxy)-2-methyl-2H-pyrazolo[4,3-f]quinazolin-9-yl)-1,4-oxazepane (150 mg, 0.29 mmol), tert-butyl (3-cyano-4-(4,4,5,5-tetramethyl-1,3,2-dioxaborolan-2-yl)benzo[b]thiophen-2-yl)carbamate (247.7 mg, 0.43 mmol), DPEphosPdCl₂ (41.3 mg, 0.058 mmol) and Cs₂CO₃ (329.3 mg, 1.01 mmol) in dioxane/H₂O (3 mL/1 mL) was stirred at 110° C. under N₂ for 3 hours. The mixture was concentrated under vacuum and purified by silica gel column chromatography (2% MeOH in DCM) to give tert-butyl N-[3-cyano-4-[2-methyl-9-(1,4-oxazepan-4-yl)-7-[[(2R,8S)-2-fluoro-1,2,3,5,6,7-hexahydropyrrolizin-8-yl]methoxy]pyrazolo[4,3-f]quinazolin-4-yl]benzothiophen-2-yl]carbamate (230 mg, 0.23 mmol, 78% yield, ~70% purity) as a yellow solid.

LC-MS (ES⁺, Method 4): 1.47 min, m/z, 713.25 [M+H]⁺.

Step B, 2-amino-4-[2-methyl-9-(1,4-oxazepan-4-yl)-7-[[(2R,8S)-2-fluoro-1,2,3,5,6,7-hexahydropyrrolizin-8-yl]methoxy]pyrazolo[4,3-f]quinazolin-4-yl]benzothiophene-3-carbonitrile. A mixture of tert-butyl (3-cyano-4-(7-(((2R,7aS)-2-fluorotetrahydro-1H-pyrrolizin-7a (5H)-yl) methoxy)-2-methyl-9-(1,4-oxazepan-4-yl)-2H-pyrazolo[4,3-f]quinazolin-4-yl)benzo[b]thiophen-2-yl)carbamate (210 mg, 0.29 mmol) in DCM/TFA (3 mL/1 mL) was stirred at 25° C. for 1.5 hours. NaHCO₃ (saturated aqueous solution) (20 mL) was added to adjust the pH to 7-8 at 0° C. The mixture was poured into water (60 mL) and extracted with 3:1 CHCl₃/IPA (3×30 mL). The combined organic layers were washed with brine (40 mL), dried over Na₂SO₄ and concentrated under vacuum to give 170 mg of crude material purified by preparative TLC (10% MeOH in DCM) affording 2-amino-4-[2-methyl-9-(1,4-oxazepan-4-yl)-7-[[(2R,8S)-2-fluoro-1,2,3,5,6,7-hexahydropyrrolizin-8-yl]methoxy]pyrazolo[4,3-f]quinazolin-4-yl]benzothiophene-3-carbonitrile (example 3), as a white solid (25.7 mg, 0.042 mmol, 14% yield).

LC-MS (ES⁺, Method 4): 1.067 min, m/z, 613.20 [M+H]⁺.

¹H NMR (400 MHz, DMSO-d6) δ/ppm: 8.48 (s, 1H), 7.76 (dd, J=7.8, 1.2 Hz, 1H), 7.60 (s, 2H), 7.31-7.19 (m, 3H), 5.45 (d, J=53.4 Hz, 1H), 4.34 (s, 2H), 4.12 (s, 3H), 3.77 (m, 9H), 3.47 (s, 2H), 3.18-3.06 (m, 1H), 2.42-2.11 (m, 3H), 2.00 (s, 5H).

The following examples in table 19 were made by analogy with example 3 (scheme 3) starting from the intermediate and building blocks for step A as described in the table. The catalyst DPEphosPdCl₂ could be substituted for Pd(dtbpf)Cl₂ in step A. Step B was not required where no protecting groups were present on the step A coupling partner.

TABLE 19

| | | UPLC-MS | |
|---|---|---|---|
| Step A, intermediate + | | (ES⁺): min, | ¹H NMR (400 MHz, |
| building block | Example Structure/Name | m/z | DMSO-d₆) δ/ppm |
| Intermediate I-1, | Example 4 | 2.29 | 9.86 (s, 1H), 8.43 |
| and 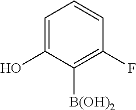 [SMILES: OB(O)c1c(O)cccc1F] 2-fluoro-6- hydroxyphenyl)boronic acid | 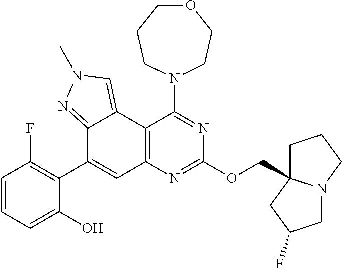 [SMILES: Cn1cc2c(n1)c(-c1c(O)cccc1F)cc1nc(OC[C@@]34CCCN3C[C@H](F)C4)nc(N3CCCOCC3)c12] | min, 551.4 [M + H]⁺ (Method 3) | Hz, 1H), 5.36 (d, J = 3.6 Hz, 1H), 5.22 (dd, J = 4.1, 2.2 Hz, 1H), 4.14 (s, 3H), 4.10 (s, 1H), 4.03 (d, J = 10.3 Hz, 1H), 3.83 (d, J = 5.5 Hz, 2H), 3.79 (s, 2H), 3.75 (d, J = 5.7 Hz, 2H), 3.13- 3.09 (m, 2H), 3.03 (d, J = 2.1 Hz, 1H), 2.83 (dd, J = 9.5, 6.0 Hz, 1H), 2.01 (d, J = 5.4 Hz, 5H), 1.82 (m, 3H). |
| | Example 4 F | | |
| | 3-fluoro-2-[2-methyl-9-(1,4- | | |
| | oxazepan-4-yl)-7-[[(2R,8S)-2- | | |
| | fluoro-1,2,3,5,6,7- | | |
| | hexahydropyrrolizin-8- | | |
| | yl]methoxy]pyrazolo[4,3- | | |
| | f]quinazolin-4-yl]phenol | | |
| Intermediate I-1, | Example 5 | 9.59 | 9.59 (s, 1H), 8.56 |
| and 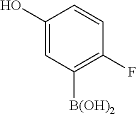 [SMILES: OB(O)c1cc(O)ccc1F] 2-fluoro-5- hydroxyphenyl)boronic acid | 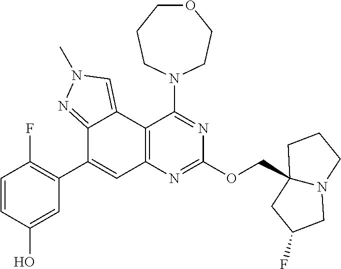 [SMILES: Cn1cc2c(n1)c(-c1cc(O)ccc1F)cc1nc(OC[C@@]34CCCN3C[C@H](F)C4)nc(N3CCCOCC3)c12] | min, 551.4 [M + H]⁺ (Method 4) | (s, 1H), 7.45 (d, J = 1.5 Hz, 1H), 7.21- 7.11 (m, 2H), 6.87 (dt, J = 8.9, 3.5 Hz, 1H), 5.68-5.49 (m, 1H), 4.58 (d, J = 3.8 Hz, 2H), 4.21 (s, 3H), 3.88 (d, J = 5.8 Hz, 6H), 3.76 (d, J = 5.6 Hz, 5H), 3.35-3.29 (m, 1H), 2.57 (d, J = 20.6 Hz, 1H), 2.38- 1.97 (m, 7H). |
| | Example 5 | | |
| | 4-fluoro-3-[2-methyl-9-(1,4- | | |
| | oxazepan-4-yl)-7-[(2R,8S)-2- | | |
| | fluoro-1,2,3,5,6,7- | | |
| | hexahydropyrrolizin-8- | | |
| | yl]methoxy]pyrazolo[4,3- | | |
| | f]quinazolin-4-yl]phenol | | |
| Intermediate I-28 and | Example 60 | 2.46 | 12.89 (s, 1H), 8.47 |
| 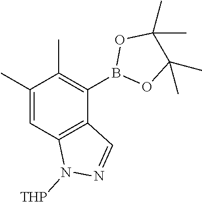 5,6-dimethyl-1- tetrahydropyran-2-yl- 4-(4,4,5,5-tetramethyl- 1,3,2-dioxaborolan-2- yl)indazole | 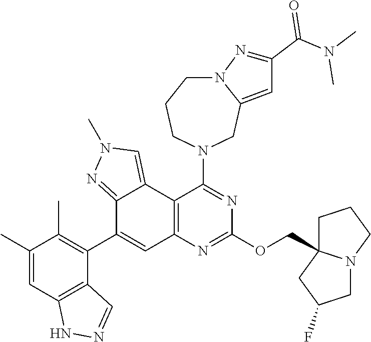 [SMILES: Cc1cc2[nH]ncc2c(-c2cc3nc(OC[C@@]45CCCN4C[C@H](F)C5)nc(N4CCCn5nc(C(=O)N(C)C)cc5C4)c3c3cn(C)nc23)c1C] | min, 692.8 [M + H]⁺ (Method 1) | (s, 1H), 7.44 (s, 1H), 7.38 (s, 1H), 7.28 (s, 1H), 6.42 (s, 1H), 5.27 (d, J = 52.3 Hz, 1H), 4.83 (s, 2H), 4.50 (s, 2H), 4.10 (s, 3H), 4.05-3.91 (m, 4H), 3.27 (s, 3H), 3.10- 2.82 (m, 8H), 2.45 (s, 3H), 2.33-1.99 (m, 8H), 1.84-1.77 (m, 2H) |
| | Example 60 | | |
| | 5-[4-(5,6-dimethyl-1H-indazol- | | |
| | 4-yl)-2-methyl-7-[[(2R,8S)-2- | | |
| | fluoro-1,2,3,5,6,7- | | |
| | hexahydropyrrolizin-8- | | |
| | yl]methoxy]pyrazolo[4,3- | | |
| | f]quinazolin-9-yl]-N,N-dimethyl- | | |
| | 4,6,7,8-tetrahydropyrazolo[1,5- | | |
| | a][1,4]diazepine-2- | | |
| | carboxamide | | |
| Intermediate I-28 | Example 62 | 2.91 min, | 10.82 (s, 1H), 8.51 |
| and 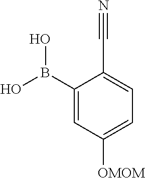 [2-cyano-5- (methoxymethoxy)phe- nyl]boronic acid | 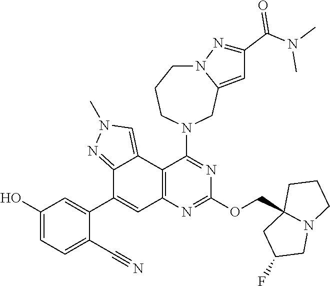 [SMILES: CN(C)C(=O)c1cc2n(n1)CCCN(c1nc(OC[C@@]34CCCN3C[C@H](F)C4)nc3cc(-c4cc(O)ccc4C#N)c4nn(C)cc4c13)C2] | 665.3 [M + H]⁺ (Method 1) | (s, 1H), 7.81 (d, J = 8.6 Hz, 1H), 7.45 (s, 1H), 7.19 (d, J = 2.4 Hz, 1H), 7.01 (dd, J = 2.4, 8.6 Hz, 1H), 6.40 (s, 1H), 5.27 (d, J = 54.2 Hz, 1H), 4.82 (s, 2H), 4.49-4.47 (m, 2H), 4.19 (s, 2H), 4.03-3.93 (m, 4H), 3.26 (s, 3H), 3.08- 2.79 (m, 7H), 2.14- 1.97 (m, 6H), 1.83- 1.75 (m, 3H) |
| | Example 62 | | |
| | 5-[4-(2-cyano-5-hydroxy- | | |
| | phenyl)-2-methyl-7-[(2R,8S)- | | |
| | 2-fluoro-1,2,3,5,6,7- | | |
| | hexahydropyrrolizin-8- | | |
| | yl]methoxy]pyrazolo[4,3- | | |
| | f]quinazolin-9-yl]-N,N-dimethyl- | | |
| | 4,6,7,8-tetrahydropyrazolo[1,5- | | |
| | a][1,4]diazepine-2- | | |
| | carboxamide | | |
| Intermediate I-28 | Example 74 | 3.28 min, | 12.91 (s, 1H), 8.46 |
| and 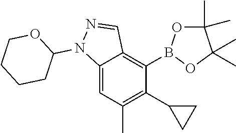 A22 5-cyclopropyl-6- metetrahydropyran-2-yl-thyl-1- 4-(4,4,5,5-tetramethyl- 1,3,2-dioxaborolan-2- yl)indazole (A-22) | 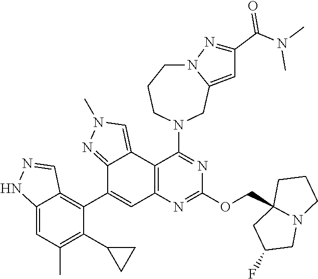 [SMILES: Cc1cc2[nH]ncc2c(-c2cc3nc(OC[C@@]45CCCN4C[C@H](F)C5)nc(N4CCCn5nc(C(=O)N(C)C)cc5C4)c3c3cn(C)nc23)c1C1CC1] | 718.4 [M + H]⁺ (Method 6) | (s, 1H), 7.45 (s, 1H), 7.42 (s, 1H), 7.33 (s, 1H), 6.31 (s, 1H), 5.37-5.17 (m, 1H), 4.94-4.87 (m, 1H), 4.85-4.77 (m, 1H), 4.55-4.48 (m, 2H), 4.11 (s, 3H), 4.06-3.86 (m, 5H), 3.26 (s, 3H), 3.14-3.02 (m, 2H), 3.02-2.98 (m, 1H), 2.93 (s, 3H), 2.86- 2.79 (m, 1H), 2.61 (s, 3H), 2.23-1.96 (m, 6H), 1.92-1.73 (m, 4H), 0.58-0.48 |
| | Example 74 | | (m, 1H), 0.39-0.29 |
| | 5-[4-(5-cyclopropyl-6-methyl- | | (m, 1H). |
| | 1H-indazol-4-yl)-2-methyl-7- | | |
| | [[(2R,8S)-2-fluoro-1,2,3,5,6,7- | | |
| | hexahydropyrrolizin-8- | | |
| | yl]methoxy]pyrazolo[4,3- | | |
| | f]quinazolin-9-yl]-N,N-dimethyl- | | |
| | 4,6,7,8-tetrahydropyrazolo[1,5- | | |
| | a][1,4]diazepine-2- | | |
| | carboxamide | | |
| Intermediate I-28 | Example 76 | 2.65 min, | 8.47 (s, 1H), 7.77 |
| and 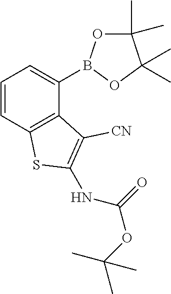 tert-butyl (3-cyano-4- (4,4,5,5-tetramethyl- | 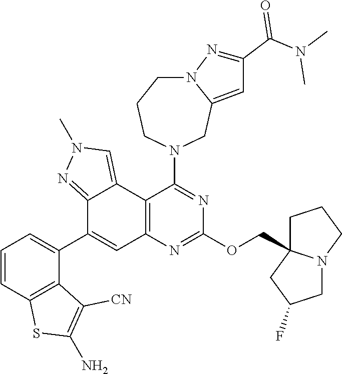 [SMILES: CN(C)C(=O)c1cc2n(n1)CCCN(c1nc(OC[C@@]34CCCN3C[C@H](F)C4)nc3cc(-c4cccc5sc(N)c(C#N)c45)c4nn(C)cc4c13)C2] | 720.7 [M + H]+ (Method 1) | (d, J = 8.9 Hz, 1H), 7.61 (s, 2H), 7.32 (d, J = 6.9 Hz, 1H), 7.27 (s, 1H), 7.23 (t, J = 7.9 Hz, 1H), 6.23 (s, 1H), 5.26 (d, J = 54.9 Hz, 1H), 4.94-4.72 (m, 2H), 4.51 (s, 2H), 4.12 (s, 3H), 4.03 (d, J = 10.7 Hz, 2H), 3.96 (d, J = 11.2 Hz, 1H), 3.87-3.81 (m, 1H), 3.20 (s, 3H), 3.10-3.06 (m, 2H), 3.00 (s, 1H), 2.89 (s, 3H), 2.85-2.79 (m, 1H), 2.30-2.23 (m, 1H), 2.13-2.07 (m, 2H), 2.04-1.96 (m, 2H), 1.85-1.75 (m, 3H). |
| 1,3,2-dioxaborolan-2- | Example 76 | | |
| yl)benzo[b]thiophen- | 5-[4-(2-amino-3-cyano- | | |
| 2-yl)carbamate | benzothiophen-4-yl)-7- | | |
| | [[(2R,8S)-2-fluoro-1,2,3,5,6,7- | | |
| | hexahydropyrrolizin-8- | | |
| | yl]methoxy]-2-methyl- | | |
| | pyrazolo[4,3-f]quinazolin-9-yl]- | | |
| | N,N-dimethyl-4,6,7,8- | | |
| | tetrahydropyrazolo[1,5- | | |
| | a][1,4]diazepine-2- | | |
| | carboxamide | | |
| Intermediate I-28 | Example 82 | 3.73 min, | 10.20 (s, 1H), 8.50 |
| and 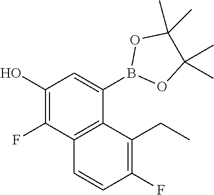 A-23 5-ethyl-1,6-difluoro-4- (4,4,5,5-tetramethyl- | 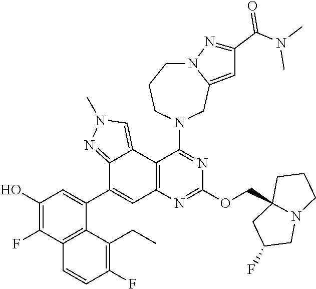 [SMILES: CCc1c(F)ccc2c(F)c(O)cc(-c3cc4nc(OC[C@@]56CCCN5C[C@H](F)C6)nc(N5CCCn6nc(C(=O)N(C)C)cc6C5)c4c4cn(C)nc34)c12] | 754.3 [M + H]+ (Method 6) | (s, 1H), 8.00-7.94 (m, 1H), 7.52-7.44 (m, 1H), 7.38 (s, 1H), 7.20-7.14 (m, 1H), 6.23 (s, 1H), 5.39-5.18 (m, 1H), 4.99-4.90 (m, 1H), 4.84-4.76 (m, 1H), 4.56-4.48 (m, 2H), 4.08 (s, 3H), 4.06- 4.00 (m, 1H), 4.00- 3.95 (m, 1H), 3.92- 3.84 (m, 1H), 3.26 (s, 3H), 3.14-2.98 (m, 3H), 2.93 (s, 3H), 2.87-2.78 (m, 1H), 2.47-2.37 (m, 2H), 2.28-2.19 (m, |
| 1,3,2-dioxaborolan-2- | Example 82 | | 1H), 2.15-1.96 (m, |
| yl)naphthalen-2-ol (A- | 5-[4-(8-ethyl-4,7-difluoro-3- | | 5H), 1.87-1.72 (m, |
| 23) | hydroxy-1-naphthyl)-2-methyl- | | 3H), 0.56 (t, J = 7.2 |
| | 7-[[(2R,8S)-2-fluoro- | | Hz, 3H). |
| | 1,2,3,5,6,7- | | |
| | hexahydropyrrolizin-8- | | |
| | yl]methoxy]pyrazolo[4,3- | | |
| | f]quinazolin-9-yl]-N,N-dimethyl- | | |
| | 4,6,7,8-tetrahydropyrazolo[1,5- | | |
| | a][1,4]diazepine-2- | | |
| | carboxamide | | |
| Intermediate I-82 and | Example 86 | 2.76 min, | 8.34 (s, 1H), 7.78- |
| 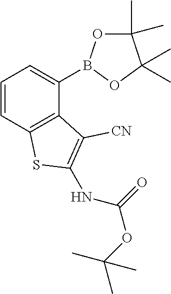 | 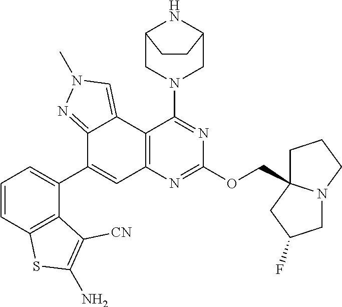 [SMILES: Cn1cc2c(n1)c(-c1cccc3sc(N)c(C#N)c13)cc1nc(OC[C@@]34CCCN3C[C@H](F)C4)nc(N3CC4CCC(C3)N4)c12] | 624.2 [M + H]+ (Method 6) | 7.76 (d, J = 7.32, 1H), 7.61 (br s, 2H), 7.31-7.29 (m, 2H), 7.26-7.20 (m, 1H), 5.43 (d, J = 53.69, 1H), 4.45- 4.02 (m, 9H), 3.76-3.73 (m, 2H), 3.13-2.96 (m, 2H), 2.38-2.11 (m, 4H), 2.01-1.92 (m, 8H). Exchangeable proton not seen. |
| tert-butyl (3-cyano-4- | Example 86 | | |
| (4,4,5,5-tetramethyl- | 2-amino-4-[9-(3,8- | | |
| 1,3,2-dioxaborolan-2- | diazabicyclo[3.2.1]octan-3-yl)- | | |
| yl)benzo[b]thiophen- | 7-[[(2R,8S)-2-fluoro- | | |
| 2-yl)carbamate | 1,2,3,5,6,7- | | |
| | hexahydropyrrolizin-8- | | |
| | yl]methoxy]-2-methyl- | | |
| | pyrazolo[4,3-f]quinazolin-4- | | |
| | yl]benzothiophene-3- | | |
| | carbonitrile | | |
| Intermediate I-79 and | Example 101 | 3.43 min, | 10.58 (s, 1H), 8.43 |
| 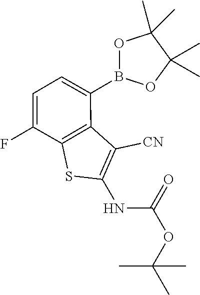 | 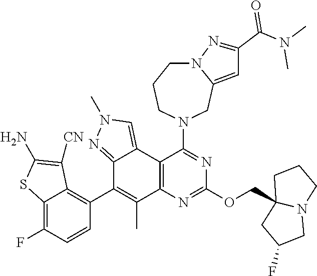 [SMILES: Cc1c(-c2ccc(F)c3sc(N)c(C#N)c23)c2nn(C)cc2c2c(N3CCCn4nc(C(=O)N(C)C)cc4C3)nc(OC[C@@]34CCCN3C[C@H](F)C4)nc12] | 752.3 [M + H]+ (Method 6) | (s, 1H), 7.87 (s, 2H), 7.23-7.19 (m, 1H), 7.15-7.11 (m, 1H), 6.30 (s, 1H), 5.41 (m, 1H), 4.89- 4.75 (m, 2H), 4.50- 4.48 (m, 3H), 4.08 (s, 3H), 4.02 (m, 2H), 3.88 (m, 2H), 3.21 (s, 3H), 2.91 (s, 3H), 2.25 (s, 3H), 2.16-1.89 (m, 9H), 1.37-1.33 (m, 1H), 1.23-1.19 (m, 1H) |
| A-26 | Example 101 | | |
| tert-butyl N-[3-cyano- | 5-[4-(2-amino-3-cyano-7- | | |
| 7-fluoro-4-(4,4,5,5- | fluoro-benzothiophen-4-yl)-7- | | |
| tetramethyl-1,3,2- | [[(2R,8S)-2-fluoro-1,2,3,5,6,7- | | |
| dioxaborolan-2- | hexahydropyrrolizin-8- | | |
| yl)benzothiophen-2- | yl]methoxy]-2,5-dimethyl- | | |
| yl]carbamate (A-26) | pyrazolo[4,3-f]quinazolin-9-yl]- | | |
| | N,N-dimethyl-4,6,7,8- | | |
| | tetrahydropyrazolo[1,5- | | |
| | a][1,4]diazepine-2- | | |
| | carboxamide | | |
| Intermediate I-28 and | Example 104 | 3.94 min, | 8.50 (s, 1H), 8.24 |
| 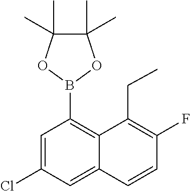 A-30 2-(3-chloro-8-ethyl-7- fluoro-1-naphthyl)- 4,4,5,5-tetramethyl- 1,3,2-dioxaborolane | 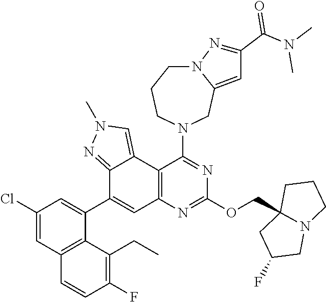 [SMILES: CCc1c(F)ccc2cc(Cl)cc(-c3cc4nc(OC[C@@]56CCCN5C[C@H](F)C6)nc(N5CCCn6nc(C(=O)N(C)C)cc6C5)c4c4cn(C)nc34)c12] | 754.3 [M + H]+ (Method 6) | (d, J = 2.4 Hz, 1H), 8.02 (dd, J = 9.2, 6.1 Hz, 1H), 7.54 (appt t, J = 9.4 Hz, 1H), 7.49-7.42 (m, 2H), 6.23 (s, 1H), 5.38-5.15 (m, 1H), 4.94 (d, J = 16.0 Hz, 1H), 4.80 (d, J = 15.9 Hz, 1H), 4.60-4.44 (m, 2H), 4.14-3.81 (m, 7H), 3.25 (s, 3H), 3.15- 2.96 (m, 3H), 2.92 (s, 3H), 2.88-2.76 (m, 1H), 2.48-2.38 (m, 1H), 2.29-1.90 (m, 6H), 1.88-1.69 (m, 3H), 0.57 (t, J = |
| | Example 104 | | 7.2 Hz, 3H) |
| | 5-[4-(3-chloro-8-ethyl-7-fluoro- | | |
| | 1-naphthyl)-2-methyl-7- | | |
| | [[(2R,8S)-2-fluoro-1,2,3,5,6,7- | | |
| | hexahydropyrrolizin-8- | | |
| | yl]methoxy]pyrazolo[4,3- | | |
| | f]quinazolin-9-yl]-N,N-dimethyl- | | |
| | 4,6,7,8-tetrahydropyrazolo[1,5- | | |
| | a][1,4]diazepine-2- | | |
| | carboxamide | | |
| Intermediate I-116 and | Example 173 | 2.89 min, | 13.21(s, 1H), |
| 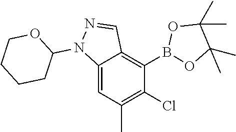 5-chloro-6-methyl-1- tetrahydropyran-2-yl- 4-(4,4,5,5-tetramethyl- 1,3,2-dioxaborolan-2- | 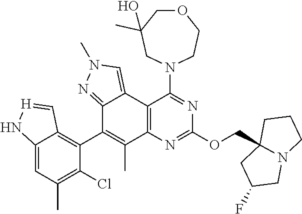 | 649.4 [M + H]+ (Method 1) | 8.42 (s, 1H), 7.63 (s, 1H), 7.35-7.32(d, J = 12.80, 1H), 5.81 (br, 1H), 5.35- 5.22 (d, J = 54.97, 1H), 4.22-4.16 (m, 1H), 4.11-4.08 (m, 1H), 4.04 (m, 3H), 3.96-3.93 (m, 1H), 3.84-3.71 (m, 4H), 3.63-3.56 (m, 2H), 3.11-3.08 (m, 2H), 3.02 (m, 1H), |
| yl)indazole | Example 173 | | 2.86-2.80 (m, 1H), |
| | 4-[4-(5-chloro-6-methyl-1H- | | 2.55-2.54 (m, 3H), |
| | indazol-4-yl)-2,5-dimethyl-7- | | 2.21-2.20 (d, |
| | [[(2R,8S)-2-fluoro-1,2,3,5,6,7- | | J = 2.60, 3H), 2.08- |
| | hexahydropyrrolizin-8- | | 1.98 (m, 3H), 1.87- |
| | yl]methoxy]pyrazolo[4,3- | | 1.77 (m, 3H), |
| | f]quinazolin-9-yl]-6-methyl-1,4- | | 1.23 (s, 1H), |
| | oxazepan-6-ol | | 1.08 (m, 3H). |

### Example 16, 4-methyl-6-[2-methyl-9-(1,4-oxazepan-4-yl)-7-[(2R,8S)-2-fluoro-1,2,3,5,6,7-hexahydropyrrolizin-8-yl]methoxy]pyrazolo[4,3-f]quinazolin-4-yl]-5-(trifluoromethyl)pyridin-2-amine

Example 16 was prepared according to scheme 4 below.

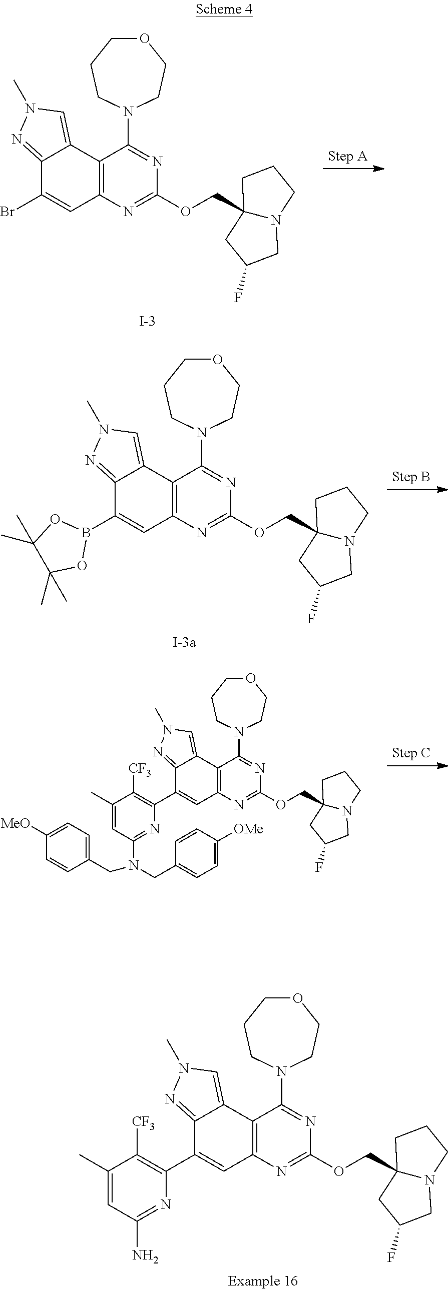

Step A, 4-[2-methyl-7-[[(2R,8S)-2-fluoro-1,2,3,5,6,7-hexahydropyrrolizin-8-yl]methoxy]-4-(4,4,5,5-tetramethyl-1,3,2-dioxaborolan-2-yl)pyrazolo[4,3-f]quinazolin-9-yl]-1,4-oxazepane. A mixture of Pd₂(dba)₃ (70.52 mg, 0.08 mmol), KOAc (113.36 mg, 1.16 mmol), bis(pinacolato)diboron (575.17 mg, 1.93 mmol), X-phos (183.56 mg, 0.39 mmol) and 4-[4-bromo-2-methyl-7-[[(2R,8S)-2-fluoro-1,2,3,5,6,7-hexahydropyrrolizin-8-yl]methoxy]pyrazolo[4,3-f]quinazolin-9-yl]-1,4-oxazepane (1-3) (200 mg, 0.39 mmol) in THF (30 mL) was stirred at 75° C. under N₂ overnight. The mixture was concentrated under vacuum and purified by silica gel column (5% MeOH in DCM) to give 4-[2-methyl-7-[[(2R,8S)-2-fluoro-1,2,3,5,6,7-hexahydropyrrolizin-8-yl]methoxy]-4-(4,4,5,5-tetramethyl-1,3,2-dioxaborolan-2-yl)pyrazolo[4,3-f]quinazolin-9-yl]-1,4-oxazepane (I-3a) (140 mg, 64% yield) as a white solid.

LC-MS (ES⁺, Method 4): 0.46 min, m/z, 485.20 [M+H]⁺ (boronic acid fragment).

¹H NMR (400 MHz, DMSO-d6) δ/ppm: 8.46 (s, 1H), 7.80 (s, 1H), 5.35-5.21 (d, J=54.0 Hz, 1H), 4.23 (s, 3H), 4.12-4.00 (m, 2H), 3.81-3.72 (m, 8H), 3.11-3.02 (m, 3H), 2.86-2.80 (m, 1H), 2.14-1.97 (m, 8H), 1.82 (s, 12H), 1.74-1.70 (m, 2H).

Step B, N,N-bis[(4-methoxyphenyl)methyl]-4-methyl-6-[2-methyl-9-(1,4-oxazepan-4-yl)-7-[[2R,8S)-2-fluoro-1,2,3,5,6,7-hexahydropyrrolizin-8-yl]methoxy]pyrazolo[4,3-f]quinazolin-4-yl]-5-(trifluoromethyl)pyridin-2-amine. A mixture of 4-(7-(((2R,7aS)-2-fluorotetrahydro-1H-pyrrolizin-7a (5H)-yl) methoxy)-2-methyl-4-(4,4,5,5-tetramethyl-1,3,2-dioxaborolan-2-yl)-2H-pyrazolo[4,3-f]quinazolin-9-yl)-1,4-oxazepane (I-3a) (20 mg, 0.035 mmol), 6-chloro-N,N-bis(4-methoxybenzyl)-4-methyl-5-(trifluoromethyl)pyridin-2-amine (15.9 mg, 0.035 mmol), CataCXiumAPdG3 (5.14 mg, 0.007 mmol) and Cs₂CO₃ (40.26 mg, 0.123 mmol) in toluene/H₂O (1.2 mL/0.3 mL) was stirred at 80° C. under N₂ for 3 hours. The mixture was cooled to room temperature, filtered through celite and concentrated to a brown oil, purified by flash column chromatography on silica gel (1% MeOH in DCM) to afford N, N-bis[(4-methoxyphenyl)methyl]-4-methyl-6-[2-methyl-9-(1,4-oxazepan-4-yl)-7-[[2R,8S)-2-fluoro-1,2,3,5,6,7-hexahydropyrrolizin-8-yl]methoxy]pyrazolo[4,3-f]quinazolin-4-yl]-5-(trifluoromethyl)pyridin-2-amine (110 mg, 53% yield) as a yellow solid.

LC-MS (ES⁺, Method 4): 1.867 min, m/z, 855.30 [M+H]⁺.

Step C, 4-methyl-6-[2-methyl-9-(1,4-oxazepan-4-yl)-7-[[(2R,8S)-2-fluoro-1,2,3,5,6,7-hexahydropyrrolizin-8-yl]methoxy]pyrazolo[4,3-f]quinazolin-4-yl]-5-(trifluoromethyl)pyridin-2-amine. A mixture of N, N-bis[(4-methoxyphenyl)methyl]-4-methyl-6-[2-methyl-9-(1,4-oxazepan-4-yl)-7-[[2R,8S)-2-fluoro-1,2,3,5,6,7-hexahydropyrrolizin-8-yl]methoxy]pyrazolo[4,3-f]quinazolin-4-yl]-5-(trifluoromethyl)pyridin-2-amine (100 mg, 0.117 mmol) in toluene/methanesulfonic acid/AcOH (8/2/2 mL) was stirred at 25° C. and overnight. NaHCO₃ saturated aqueous solution (80 mL) was added to adjust the pH to 7-8 at 0° C. The mixture was poured into water (100 mL) and extracted with EtOAc (3×50 mL). The combined organic layers were washed with brine (80 mL), dried over Na₂SO₄ and concentrated in vacuum before purification by reverse phase preparative HPLC by pre-HPLC to afford 4-methyl-6-[2-methyl-9-(1,4-oxazepan-4-yl)-7-[[(2R,8S)-2-fluoro-1,2,3,5,6,7-hexahydropyrrolizin-8-yl]methoxy]pyrazolo[4,3-f]quinazolin-4-yl]-5-(trifluoromethyl)pyridin-2-amine (12.4 mg, 17% yield) as a yellow solid.

LC-MS (ES⁺, Method 4): 1.067 min, m/z, 615.20 [M+H]⁺.

¹H NMR (400 MHz, DMSO-d6) δ/ppm: 8.42 (s, 1H), 7.09 (s, 1H), 6.65 (s, 2H), 6.46 (s, 1H), 5.29 (m, 1H), 4.13 (s, 3H), 4.12-4.08 (m, 1H), 4.05-4.00 (m, 1H), 3.77 (m, 8H), 3.15-3.08 (m, 2H), 3.04 (m, 1H), 2.83 (dd, J=9.3, 6.2 Hz, 1H), 2.37 (d, J=2.2 Hz, 3H), 2.21-2.12 (m, 1H), 2.09-1.98 (m, 4H), 1.89-1.76 (m, 3H).

The following examples in table 20 were made by analogy with example 16 (scheme 4) starting from the intermediate and building blocks for step B as described in the table. Step C was not required where no protecting groups were present on the step B coupling partner.

TABLE 20

| | | UPLC-MS | |
|---|---|---|---|
| Step B, intermediate + | | (ES+): min, | ¹H NMR (400 MHz, |
| building block | Example Structure/Name | m/z | DMSO-d₆) δ/ppm |
| Intermediate I-3a, | Example 38 | 0.97 min, | 9.93 (s, 1H), 8.48 |
| and 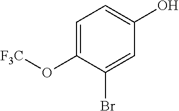 [SMILES: Oc1ccc(OC(F)(F)F)c(Br)c1] 3-Bromo-4- (trifluoromethoxy)phe- nol | 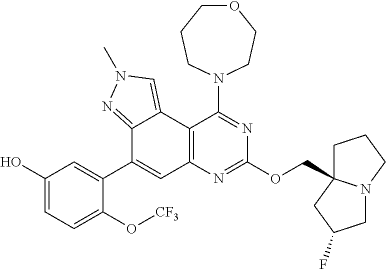 [SMILES: Cn1cc2c(n1)c(-c1cc(O)ccc1OC(F)(F)F)cc1nc(OC[C@@]34CCCN3C[C@H](F)C4)nc(N3CCCOCC3)c12] | 617.2 [M + H]⁺ (Method 4) | (s, 1H), 7.37-7.27 (m, 2H), 7.06 (d, J = 3.0 Hz, 1H), 6.93 (dd, J = 8.9, 3.0 Hz, 1H), 5.30 (d, J = 53.8 Hz, 1H), 4.19- 4.11 (m, 4H), 4.04 (d, J = 10.4 Hz, 1H), 3.83- 3.74 (m, 8H), 3.15- 3.04 (m, 3H), 2.89-2.82 (m, 1H), 2.17-1.98 (m, 5H), 1.89- 1.77 (m, 3H). |
| | Example 38 | | |
| | 3-[2-methyl-9-(1,4-oxazepan- | | |
| | 4-yl)-7-[[(2R,8S)-2-fluoro- | | |
| | 1,2,3,5,6,7- | | |
| | hexahydropyrrolizin-8- | | |
| | yl]methoxy]pyrazolo[4,3- | | |
| | f]quinazolin-4-yl]-4- | | |
| | (trifluoromethoxy)phenol | | |
| Intermediate I-3a, | Example 39 | 2.72 min, | 9.38 (s, 1H), 8.49 |
| and 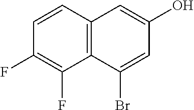 [SMILES: Oc1cc(Br)c2c(F)c(F)ccc2c1] 4-bromo-5,6-difluoro- naphthalen-2-ol | 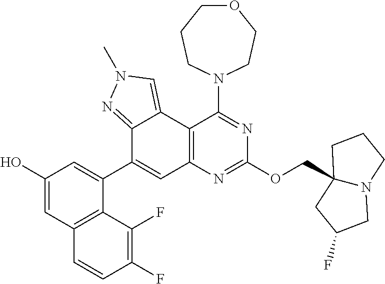 [SMILES: Cn1cc2c(n1)c(-c1cc(O)cc3ccc(F)c(F)c13)cc1nc(OC[C@@]34CCCN3C[C@H](F)C4)nc(N3CCCOCC3)c12] | 619.5 [M + H]⁺ (Method 4) | (s, 1H), 7.70 (dd, J = 9.4, 4.9 Hz, 1H), 7.52 (td, J = 9.6, 7.4 Hz, 1H), 7.34 (d, J = 10.3 Hz, 2H), 7.18 (d, J = 2.2 Hz, 1H), 5.54 (s, 1H), 4.41 (s, 2H), 4.08 (s, 3H), 3.88 (s, 1H), 3.84 (d, J = 5.0 Hz, 2H), 3.79-3.76 (m, 2H), 3.59 (p, J = 6.5 Hz, 4H), 3.11 (q, J = 7.3 Hz, 3H), 2.46-2.27 (m, 2H), 2.25-2.17 (m, 1H), 2.12- |
| | Example 39 | | 1.95 (m, 5H). |
| | 5,6-difluoro-4-[2-methyl-9-(1,4- | | |
| | oxazepan-4-yl)-7-[(2R,8S)-2- | | |
| | fluoro-1,2,3,5,6,7- | | |
| | hexahydropyrrolizin-8- | | |
| | yl]methoxy]pyrazolo[4,3- | | |
| | f]quinazolin-4-yl]naphthalen-2- | | |
| | ol | | |

### Example 106, 2-amino-7-fluoro-4-[2-methyl-7-[[(2R,8S)-2-fluoro-1,2,3,5,6,7-hexahydropyrrolizin-8-yl]methoxy]-9-[(3R)-3-hydroxy-3-methyl-1-piperidyl]pyrazolo[4,3-f]quinazolin-4-yl]thieno[3,2-c]pyridine-3-carbonitrile

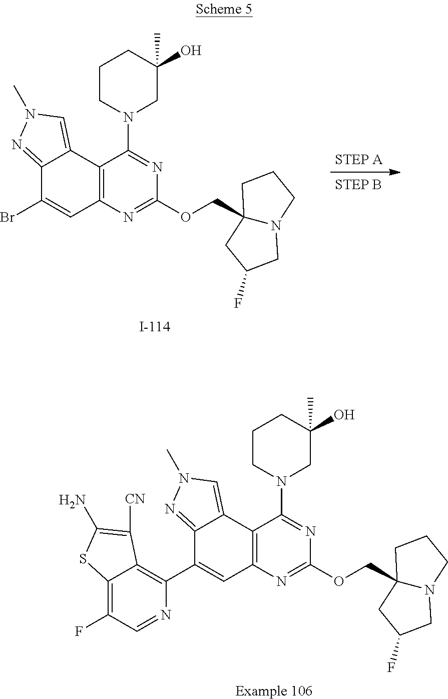 [SMILES: Cn1cc2c(n1)c(Br)cc1nc(OC[C@@]34CCCN3C[C@H](F)C4)nc(N3CCC[C@@](C)(O)C3)c12 ; "I-114" ; Cn1cc2c(n1)c(-c1ncc(F)c3sc(N)c(C#N)c13)cc1nc(OC[C@@]34CCCN3C[C@H](F)C4)nc(N3CCC[C@@](C)(O)C3)c12 ; "Example 106"]

Step A, tert-butyl N-[3-cyano-7-fluoro-4-[7-[[(2R,8S)-2-fluoro-1,2,3,5,6,7-hexahydropyrrolizin-8-yl]methoxy]-9-[(3R)-3-hydroxy-3-methyl-1-piperidyl]-2-methyl-pyrazolo[4,3-f]quinazolin-4-yl]thieno[3,2-c]pyridin-2-yl]carbamate. To a microwave vial was added a mixture of (3R)-1-[4-bromo-7-[(2R,8S)-2-fluoro-1,2,3,5,6,7-hexahydropyrrolizin-8-yl]methoxy]-2-methyl-pyrazolo[4,3-f]quinazolin-9-yl]-3-methyl-piperidin-3-ol (1-114) (73.1 mg, 0.14 mmol), Potassium acetate (40.21 mg, 0.41 mmol), Bis(neopentyl glycolato)diboron (46.47 mg, 0.21 mmol), Dichloro[bis(diphenylphosphinophenyl) ether]palladium(II) Pd 14.8% (9.74 mg, 0.01 mmol) and 1,4-Dioxane (1.4 mL). The vial was sealed and the suspension degassed with nitrogen then heated to 95° C. and left to stir for 1 hour. The reaction mixture was cooled to room temperature then water (0.45 mL) was added and the mixture degassed with nitrogen before tert-butyl N-(4-chloro-3-cyano-7-fluoro-thieno[3,2-c]pyridin-2-yl)carbamate (45.2 mg, 0.14 mmol) (A-34), XPhos Pd G2 (10.7 mg, 0.01 mmol) and potassium phosphate tribasic (43.67 mg, 0.21 mmol) were added. The resulting mixture was heated to 85° C. and left to stir for 1 hour. The reaction mixture was cooled to room temperature and partitioned between DCM and H₂O. The layers were separated and the aqueous extracted (2×DCM). The combined organics were passed through phase separator cartridge and concentrated in vacuo. The residue was purified by flash column chromatography (KP-NH column, eluting in 0-20% MeOH in DCM) to afford the desired product tert-butyl N-[3-cyano-7-fluoro-4-[7-[[(2R,8S)-2-fluoro-1,2,3,5,6,7-hexahydropyrrolizin-8-yl]methoxy]-9-[(3R)-3-hydroxy-3-methyl-1-piperidyl]-2-methyl-pyrazolo[4,3-f]quinazolin-4-yl]thieno[3,2-c]pyridin-2-yl]carbamate (17 mg, 0.0228 mmol, 17% yield) as a yellow solid.

UPLC-MS (ES⁺, Method 5): 1.89 min, m/z, 746.3 [M+H]⁺.

Step B, 2-amino-7-fluoro-4-[2-methyl-7-[[(2R,8S)-2-fluoro-1,2,3,5,6,7-hexahydropyrrolizin-8-yl]methoxy]-9-[(3R)-3-hydroxy-3-methyl-1-piperidyl]pyrazolo[4,3-f]quinazolin-4-yl]thieno[3,2-c]pyridine-3-carbonitrile. To a solution of tert-butyl N-[3-cyano-7-fluoro-4-[2-methyl-7-[(2R,8S)-2-fluoro-1,2,3,5,6,7-hexahydropyrrolizin-8-yl]methoxy]-9-[(3R)-3-hydroxy-3-methyl-1-piperidyl]pyrazolo[4,3-f]quinazolin-4-yl]thieno[3,2-c]pyridin-2-yl]carbamate (17. mg, 0.02 mmol) in DCM (0.4 mL) was added Trifluoroacetic acid (87.27 μL, 1.14 mmol) and the mixture left to stir at room temperature for 3 hours. The reaction was concentrated in vacuo and the residue was passed through SCX (1 g) and flushed off with NH₃ (1M in MeOH). The filtrate was reduced in vacuo and purified by reverse phase column chromatography (eluting in 5-95% MeCN in water (0.1% formic acid)). Product containing fractions were combined and loaded onto a pre-equilibrated SCX-2 column, washing with MEOH and eluting with 1M NH₃/MeOH to afford the desired product 2-amino-7-fluoro-4-[2-methyl-7-[[(2R,8S)-2-fluoro-1,2,3,5,6,7-hexahydropyrrolizin-8-yl]methoxy]-9-[(3R)-3-hydroxy-3-methyl-1-piperidyl]pyrazolo[4,3-f]quinazolin-4-yl]thieno[3,2-c]pyridine-3-carbonitrile (4 mg, 0.0062 mmol, 27% yield) as a yellow solid.

UPLC-MS (ES⁺, Method 1): 2.70 min, m/z, 646.2 [M+H]⁺.

¹H NMR (400 MHz, DMSO-d6) δ/ppm: 8.70-8.54 (m, 1H), 8.39 (s, 1H), 8.17 (s, 2H), 7.36 (s, 1H), 5.30 (d, J=53.0 Hz, 1H), 4.60-4.48 (m, 1H), 4.30-4.22 (m, 1H), 4.13 (s, 3H), 4.09-3.98 (m, 1H), 3.53-3.40 (m, 2H), 3.17-2.98 (m, 4H), 2.92-2.79 (m, 2H), 2.21-2.00 (m, 4H), 1.90-1.73 (m, 4H), 1.72-1.56 (m, 2H), 1.14-1.00 (m, 3H).

The following examples in table 21 were made by analogy with example 106 (scheme 5) starting from the intermediate and building blocks for step A as described in the table.

TABLE 21

| | | UPLC-MS | |
|---|---|---|---|
| Step A, intermediate + | | (ES+): min, | ¹H NMR (400 MHz, |
| building block | Example Structure/Name | m/z | DMSO-d₆) δ/ppm |
| Intermediate I-8 and | Example 107 | 2.33 min, | 8.56-8.44 (1H, m), |
| 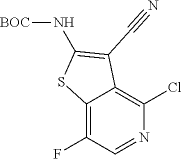 [SMILES: CC(C)(C)OC(=O)Nc1sc2c(F)cnc(Cl)c2c1C#N] A-34 tert-butyl N-(4-chloro- 3-cyano-7-fluoro- thieno[3,2-c]pyridin-2- yl)carbamate (A-34) | 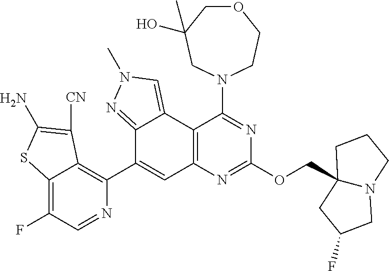 [SMILES: Cn1cc2c(n1)c(-c1ncc(F)c3sc(N)c(C#N)c13)cc1nc(OC[C@@]34CCCN3C[C@H](F)C4)nc(N3CCOCC(C)(O)C3)c12] | 662.5 [M + H]⁺ (Method 1) | 8.39 (1H, s), 8.18 (2H, s), 7.38 (1H, s), 5.67 (1H, br. s), 5.29 (1H, d, J = 54.2 Hz), 4.15-4.02 (5H, m), 3.96-3.79 (4H, m), , 3.59- 3.35 (3H, m), 3.13- 3.06 (2H, m), 3.04- 3.01 (1H, m), 2.89- 2.79 (1H, m), 2.24- 1.99 (4H, m), 1.87- 1.77 (3H, m) 1.01 (3H, s). |
| | Example 107 | | |
| | 2-amino-7-fluoro-4-[7- | | |
| | [[(2R,8S)-2-fluoro-1,2,3,5,6,7- | | |
| | hexahydropyrrolizin-8- | | |
| | yl]methoxy]-9-(6-hydroxy-6- | | |
| | methyl-1,4-oxazepan-4-yl)-2- | | |
| | methyl-pyrazolo[4,3- | | |
| | f]quinazolin-4-yl]thieno[3,2- | | |
| | c]pyridine-3-carbonitrile | | |
| Intermediate I-116 and | Example 108 | 2.55 min, | 8.42-8.39 (m, 2H), |
| 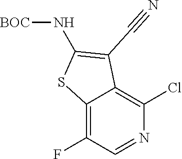 [SMILES: CC(C)(C)OC(=O)Nc1sc2c(F)cnc(Cl)c2c1C#N] A-34 tert-butyl N-(4-chloro- 3-cyano-7-fluoro- thieno[3,2-c]pyridin-2- yl)carbamate (A-34) | 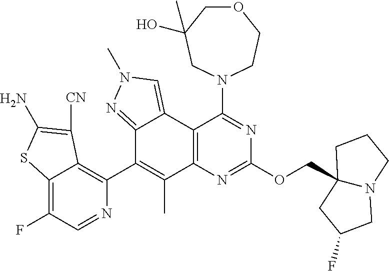 [SMILES: Cc1c(-c2ncc(F)c3sc(N)c(C#N)c23)c2nn(C)cc2c2c(N3CCOCC(C)(O)C3)nc(OC[C@@]34CCCN3C[C@H](F)C4)nc12] | 676.4 [M + H]⁺ (Method 1) | 8.18-8.16 (m, 2H), 5.28 (d, J = 54.1 Hz, 1H), 4.22-4.09 (m, 2H), 4.08-4.04 (m, 3H), 3.94-3.67 (m, 6H), 3.58-3.49 (m, 2H), 3.13-3.06 (m, 2H), 3.06-2.99 (m, 1H), 2.86-2.80 (m, 1H), 2.57-2.54 (m, 1H), 2.25-2.21 (m, 3H), 2.08-1.93 (m, 4H), 1.87-1.77 (m, 3H), 1.04-1.01 (m, 2H). |
| | Example 108 | | |
| | 2-amino-7-fluoro-4-[7- | | |
| | [[(2R,8S)-2-fluoro-1,2,3,5,6,7- | | |
| | hexahydropyrrolizin-8- | | |
| | yl]methoxy]-9-(6-hydroxy-6- | | |
| | methyl-1,4-oxazepan-4-yl)-2,5- | | |
| | dimethyl-pyrazolo[4,3- | | |
| | f]quinazolin-4-yl]thieno[3,2- | | |
| | c]pyridine-3-carbonitrile | | |

### Example 50, 5-[4-(8-ethyl-7-fluoro-3-hydroxy-1-naphthyl)-2-methyl-7-[[(2S)-azetidin-2-yl]methoxy]pyrazolo[4,3-f]quinazolin-9-yl]-N,N-dimethyl-4,6,7,8-tetrahydropyrazolo[1,5-a][1,4]diazepine-2-carboxamide

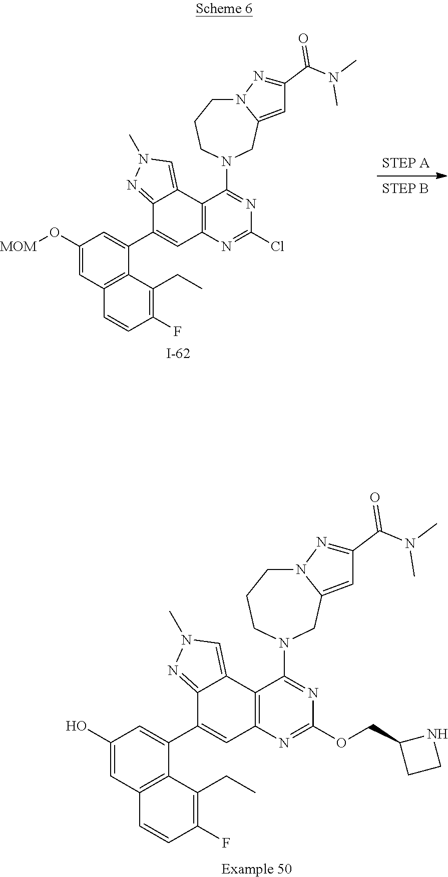

Step A, tert-butyl(2S)-2-[[9-[2-(dimethylcarbamoyl)-4,6,7,8-tetrahydropyrazolo[1,5-a][1,4]diazepin-5-yl]-4-[8-ethyl-7-fluoro-3-(methoxymethoxy)-1-naphthyl]-2-methyl-pyrazolo[4,3-f]quinazolin-7-yl]oxymethyl]azetidine-1-carboxylate. A stirring mixture of 5-[7-chloro-4-[8-ethyl-7-fluoro-3-(methoxymethoxy)-1-naphthyl]-2-methyl-pyrazolo[4,3-f]quinazolin-9-yl]-N,N-dimethyl-4,6,7,8-tetrahydropyrazolo[1,5-a][1,4]diazepine-2-carboxamide (150. mg, 0.16 mmol), (S)-1-Boc-2-Azetidinemethanol (44.88 mg, 0.24 mmol) and Cesium Carbonate (156.18 mg, 0.48 mmol) in Toluene (1 mL) was degassed with nitrogen before Palladium(II) acetate (3.59 mg, 0.02 mmol) and (+/−)-BINAP (19.9 mg, 0.03 mmol) were added. The reaction mixture was heated to 110° C. and left to stir overnight. The reaction mixture was cooled to room temperature, passed through phase separating filter paper and the solvents removed in vacuo. The crude material was purified by flash column chromatography (eluting in 0-100% EtOAc in Pet. ether) to afford the desired product tert-butyl(2S)-2-[9-[2-(dimethylcarbamoyl)-4,6,7,8-tetrahydropyrazolo[1,5-a][1,4]diazepin-5-yl]-4-[8-ethyl-7-fluoro-3-(methoxymethoxy)-1-naphthyl]-2-methyl-pyrazolo[4,3-f]quinazolin-7-yl]oxymethyl]azetidine-1-carboxylate (130 mg, 0.1609 mmol, 100% yield) as a dark yellow oil.

UPLC-MS (ES⁺, Method 5): 1.94 min, 808.4 [M+H]⁺.

Step B, 5-[4-(8-ethyl-7-fluoro-3-hydroxy-1-naphthyl)-2-methyl-7-[(2S)-azetidin-2-yl]methoxy]pyrazolo[4,3-f]quinazolin-9-yl]-N,N-dimethyl-4,6,7,8-tetrahydropyrazolo[1,5-a][1,4]diazepine-2-carboxamide. A mixture of tert-butyl (2S)-2-[9-[2-(dimethylcarbamoyl)-4,6,7,8-tetrahydropyrazolo[1,5-a][1,4]diazepin-5-yl]-4-[8-ethyl-7-fluoro-3-(methoxymethoxy)-1-naphthyl]-2-methyl-pyrazolo[4,3-f]quinazolin-7-yl]oxymethyl]azetidine-1-carboxylate (130. mg, 0.16 mmol), Triethylsilane (0.24 mL, 1.53 mmol) and Trifluoroacetic acid (1.17 mL, 15.26 mmol) in DCM (2 mL) was left to stir at room temperature for 2 hours. The reaction mixture was concentrated in vacuo and the crude material purified by reverse phase chromatography (eluting in 0-50% acetonitrile (0.1% formic acid) in water (0.1% formic acid)). Product containing fractions were loaded onto a pre-equilibrated SCX-2 column (2 g, washing with MeOH and eluting in 1N NH₃/MeOH) to afford the desired product 5-[4-(8-ethyl-7-fluoro-3-hydroxy-1-naphthyl)-2-methyl-7-[(2S)-azetidin-2-yl]methoxy]pyrazolo[4,3-f]quinazolin-9-yl]-N, N-dimethyl-4,6,7,8-tetrahydropyrazolo[1,5-a][1,4]diazepine-2-carboxamide (23.7 mg, 0.0357 mmol, 22% yield) as a pale yellow solid.

UPLC-MS (ES⁺, Method 1): 2.84 min, 664.5 [M+H]⁺.

¹H NMR (400 MHz, DMSO-d6) δ/ppm: 9.82 (br s, 1H), 8.50 (s, 1H), 7.75 (dd, J=9.2, 6.1 Hz, 1H), 7.39-7.25 (m, 3H), 7.01 (d, J=2.6 Hz, 1H), 6.20 (s, 1H), 5.00-4.90 (m, 1H), 4.85-4.74 (m, 1H), 4.61-4.42 (m, 2H), 4.30-4.20 (m, 2H), 4.11-4.08 (m, 5H), 3.92-3.67 (m, 2H), 3.51-3.43 (m, 1H), 3.25 (m, 4H), 2.92 (s, 3H), 2.44-2.35 (m, 1H), 2.31-2.19 (m, 2H), 2.18-1.99 (m, 3H), 0.56 (t, J=7.3 Hz, 3H).

The following examples in table 22 were made by analogy with example 50 (scheme 6) starting from the intermediate and building blocks for step A as described in the table.

TABLE 22

| | | UPLC- | |
|---|---|---|---|
| | | MS | |
| | | (ES+): | |
| Step A, intermediate + | | min, | ¹H NMR (400 MHz, |
| building block | Example Structure/Name | m/z | DMSO-d₆) δ/ppm |
| Intermediate I-62 and | Example 51 | 2.58 | 9.81 (s, 1H), 8.49 |
| 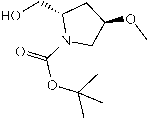 A12 tert-butyl (2S,4R)-2- (hydroxymethyl)-4- methoxy-pyrrolidine-1- carboxylate (A-12) | 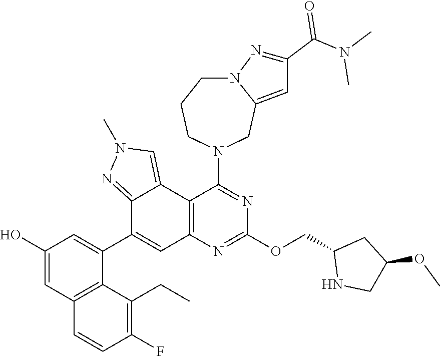 [SMILES: CCc1c(F)ccc2cc(O)cc(-c3cc4nc(OC[C@@H]5C[C@@H](OC)CN5)nc(N5CCCn6nc(C(=O)N(C)C)cc6C5)c4c4cn(C)nc34)c12] | min, 708.6 [M + H]⁺ (Meth- od 1) | (s, 1H), 7.75 (dd, J = 9.1, 6.1 Hz, 1H), 7.38-7.17 (m, 3H), 7.00 (d, J = 2.6 Hz, 1H), 6.21 (s, 1H), 5.01-4.89 (m, 1H), 4.87-4.74 (m, 1H), 4.60-4.43 (m, 2H), 4.19-3.98 (m, 6H), 3.92-3.79 (m, 2H), 3.54-3.44 (m, 1H), 3.25 (s, 3H), 3.18 (s, 3H), 2.98-2.87 (m, 4H), 2.84-2.76 (m, 1H), 2.43-2.35 (m, 1H), 2.31-2.19 (m, 1H), 2.18-1.99 (m, 2H), 1.94-1.84 (m, 1H), 1.60-1.49 (m, 1H), 0.56 (t, J = 7.3 Hz, 3H). Exchangable |
| | Example 51 | | proton not seen. |
| | 5-[4-(8-ethyl-7-fluoro-3- | | |
| | hydroxy-1-naphthyl)-2-methyl- | | |
| | 7-[(2S,4R)-4- | | |
| | methoxypyrrolidin-2- | | |
| | yl]methoxy]pyrazolo[4,3- | | |
| | f]quinazolin-9-yl]-N,N-dimethyl- | | |
| | 4,6,7,8-tetrahydropyrazolo[1,5- | | |
| | a][1,4]diazepine-2- | | |
| | carboxamide | | |
| Intermediate I-62 and | Example 52 | 3.46 | 9.80 (s, 1H), 8.47 |
| 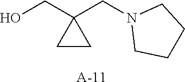 {1-[(pyrrolidin-1- yl)methyl]cyclopropyl} methanol (A-11) | 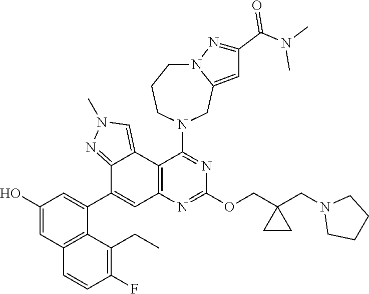 [SMILES: CCc1c(F)ccc2cc(O)cc(-c3cc4nc(OCC5(CN6CCCC6)CC5)nc(N5CCCn6nc(C(=O)N(C)C)cc6C5)c4c4cn(C)nc34)c12] | min, 732.5 [M + H]⁺ (Meth- od 6) | (s, 1H), 7.75 (dd, J = 6.1 Hz, 1H), 7.34-7.28 (m, 3H), 6.99 (d, J = 2.6 Hz, 1H), 6.24 (s, 1H), 4.95-4.77 (m, 2H), 4.57-4.47 (m, 2H), 4.16 (s, 2H), 4.07- 4.03 (m, 4H), 3.89- 3.83 (m, 1H), 3.27 (s, 3H), 2.92 (s, 3H), 2.42-2.06 (m, 10H), 1.63 (s, 4H), 0.55 (t, J = 7.4 Hz, 5H), 0.39 (s, 2H) |
| | Example 52 | | |
| | 5-[4-(8-ethyl-7-fluoro-3- | | |
| | hydroxy-1-naphthyl)-2-methyl- | | |
| | 7-[1-(pyrrolidin-1- | | |
| | ylmethyl)cyclopropyl]methoxy] | | |
| | pyrazolo[4,3-f]quinazolin-9-yl]- | | |
| | N,N-dimethyl-4,6,7,8- | | |
| | tetrahydropyrazolo[1,5- | | |
| | a][1,4]diazepine-2- | | |
| | carboxamide | | |
| Intermediate I-62 and | Example 53 | 3.10 | 9.76 (br s, 1H), |
| 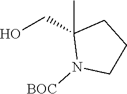 (2S)-1-BOC-2- methylpyrrolidine-2- methanol | 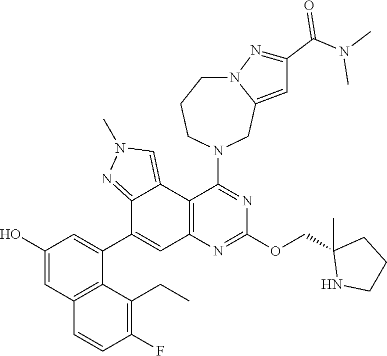 [SMILES: CCc1c(F)ccc2cc(O)cc(-c3cc4nc(OC[C@]5(C)CCCN5)nc(N5CCCn6nc(C(=O)N(C)C)cc6C5)c4c4cn(C)nc34)c12] | min, 692.5 [M + H]⁺ (Meth- od 1) | 8.29 (s, 1H), 7.736 (dd, J = 9.1, 6.2 Hz, 1H), 7.31 (t, J = 9.4 Hz, 1H), 7.27 (d, J = 2.4 Hz, 1H), 7.09 (br s, 1H), 7.00 (s, 1H), 6.28-5.92 (m, 2H), 4.94-4.60 (m, 2H), 4.51 (s, 2H), 4.03 (s, 3H), 3.98- 3.60 (m, 5H), 3.55- 3.46 (m, 1H), 3.25 (s, 3H), 2.93 (s, 3H), 2.44-2.37 (m, 1H), 2.29-1.95 (m, 3H), 1.86-1.62 (m, 4H), 1.38 (s, 3H), 0.64-0.51 (m, 3H). |
| | Example 53 | | |
| | 5-[4-(8-ethyl-7-fluoro-3- | | |
| | hydroxy-1-naphthyl)-2-methyl- | | |
| | 7-[[(2S)-2-methylpyrrolidin-2- | | |
| | yl]methoxy]pyrazolo[4,3- | | |
| | f]quinazolin-9-yl]-N,N-dimethyl- | | |
| | 4,6,7,8-tetrahydropyrazolo[1,5- | | |
| | a][1,4]diazepine-2- | | |
| | carboxamide | | |
| Intermediate I-62 and | Example 54 | 2.89 | 9.80 (d, J = 3.8 Hz, |
| 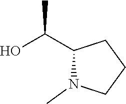 (S)-1-((S)-1- methylpyrrolidin-2- yl)ethanol | 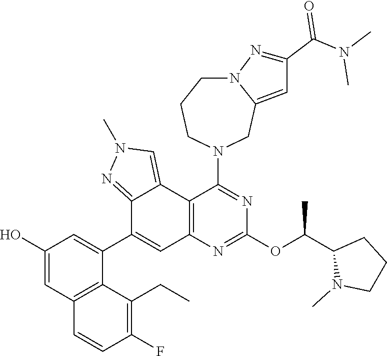 [SMILES: CCc1c(F)ccc2cc(O)cc(-c3cc4nc(O[C@@H](C)[C@@H]5CCCN5C)nc(N5CCCn6nc(C(=O)N(C)C)cc6C5)c4c4cn(C)nc34)c12] | min, 706.6 [M + H]⁺ (Meth- od 1) | 1H), 8.48 (d, J = 2.4 Hz, 1H), 7.75 (dd, J = 5.8, 8.9 Hz, 1H), 7.34-7.28 (m, 3H), 7.00 (t, J = 3.1 Hz, 1H), 6.19 (d, J = 15.8 Hz, 1H), 5.16-5.11 (m, 1H), 4.97-4.89 (m, 1H), 4.82-4.76 (m, 1H), 4.56-4.47 (m, 2H), 4.08-3.81 (m, 5H), 3.25 (s, 3H), 2.91 (s, 4H), 2.43-2.40 (m, 2H), 2.17-1.96 (m, 6H), 1.79-1.55 (m, 5H), 1.18 (d, J = 6.5 Hz, 3H), 0.57 (t, J = 7.5 Hz, 3H) |
| | Example 54 | | |
| | 5-[4-(8-ethyl-7-fluoro-3- | | |
| | hydroxy-1-naphthyl)-2-methyl- | | |
| | 7-[rac-(1S)-1-[rac-(2S)-1- | | |
| | methylpyrrolidin-2- | | |
| | yl]ethoxy]pyrazolo[4,3- | | |
| | f]quinazolin-9-yl]-N,N-dimethyl- | | |
| | 4,6,7,8-tetrahydropyrazolo[1,5- | | |
| | a][1,4]diazepine-2- | | |
| | carboxamide | | |
| Intermediate 1-62 and | Example 68 | 2.88 | 9.80 (s, 1H), 8.48 |
| 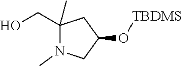 A-17 [(4R)-4-[tert- butyl(dimethyl)silyl]oxy- 1,2-dimethyl- pyrrolidin-2- yl]methanol (A-17) | 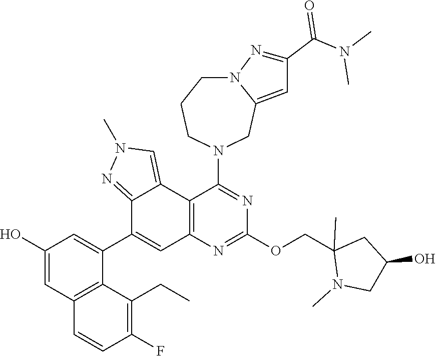 [SMILES: CCc1c(F)ccc2cc(O)cc(-c3cc4nc(OCC5(C)C[C@@H](O)CN5C)nc(N5CCCn6nc(C(=O)N(C)C)cc6C5)c4c4cn(C)nc34)c12] | min, 722.7 [M + H]⁺ (Meth- od 1) | (s, 1H), 7.75 (dd, J = 9.1, 6.1 Hz, 1H), 7.37-7.27 (m, 3H), 6.99 (d, J = 2.5 Hz, 1H), 6.23 (s, 1H), 4.94 (d, J = 15.6 Hz, 1H), 4.80 (d, J = 16.0 Hz, 1H), 4.73-4.67 (m, 1H), 4.58-4.43 (m, 2H), 4.23-4.14 (m, 1H), 4.13-3.98 (m, 6H), 3.93-3.79 (m, 1H), 3.25 (s, 3H), 3.09- 3.00 (m, 1H), 2.92 (s, 3H), 2.44-2.34 (m, 1H), 2.29-2.00 (m, 7H), 1.45 (dd, J = 13.0, 5.0 Hz, 1H), 1.09 (s, 3H), 0.55 (t, J = 7.3 Hz, 3H) |
| | Example 68 | | Exchangeable |
| | 5-[4-(8-ethyl-7-fluoro-3- | | proton not seen. |
| | hydroxy-1-naphthyl)-2-methyl- | | |
| | 7-[rac-(4R)-4-hydroxy-1,2- | | |
| | dimethyl-pyrrolidin-2- | | |
| | yl]methoxy]pyrazolo[4,3- | | |
| | f]quinazolin-9-yl]-N,N-dimethyl- | | |
| | 4,6,7,8-tetrahydropyrazolo[1,5- | | |
| | a][1,4]diazepine-2- | | |
| | carboxamide | | |
| Intermediate I-62 and | Example 69 | 2.95 | 9.80 (s, 1H), 8.48 |
| 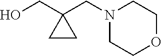 [SMILES: OCC1(CN2CCOCC2)CC1] A-18 [1- (morpholinomethyl)cy- clopropyl]methanol (A- 18) | 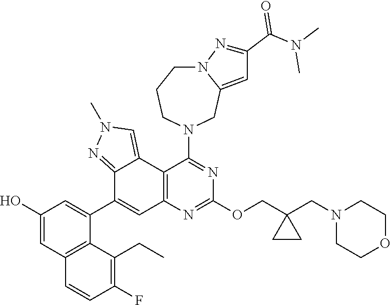 [SMILES: CCc1c(F)ccc2cc(O)cc(-c3cc4nc(OCC5(CN6CCOCC6)CC5)nc(N5CCCn6nc(C(=O)N(C)C)cc6C5)c4c4cn(C)nc34)c12] | min, 748.8 [M + H]⁺ (Meth- od 1) | (s, 1H), 7.70-7.79 (m, 1H), 7.26-7.36 (m, 3H), 6.96-7.03 (m, 1H), 6.23 (s, 1H), 4.74-4.98 (m, 2H), 4.46-4.59 (m, 2H), 4.14-4.24 (m, 2H), 4.00-4.11 (m, 4H), 3.82-3.93 (m, 1H), 3.44-3.56 (m, 4H), 3.27 (s, 3H), 2.92 (s, 3H), 2.31- 2.40 (m, 5H), 2.19- 2.30 (m, 3H), 2.01- 2.16 (m, 2H), 0.51- 0.62 (m, 5H), 0.34- 0.40 (m, 2H). |
| | Example 69 | | |
| | 5-[4-(8-ethyl-7-fluoro-3- | | |
| | hydroxy-1-naphthyl)-2-methyl- | | |
| | 7-[[1- | | |
| | (morpholinomethyl)cyclopropyl] | | |
| | methoxy]pyrazolo[4,3- | | |
| | f]quinazolin-9-yl]-N,N-dimethyl- | | |
| | 4,6,7,8-tetrahydropyrazolo[1,5- | | |
| | a][1,4]diazepine-2- | | |
| | carboxamide | | |
| Intermediate I-62 and | Example 70 | 2.87 | 9.81 (s, 1H), 8.50 |
| 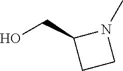 N [(2S)-1- methylazetidin-2- yl]methanol | 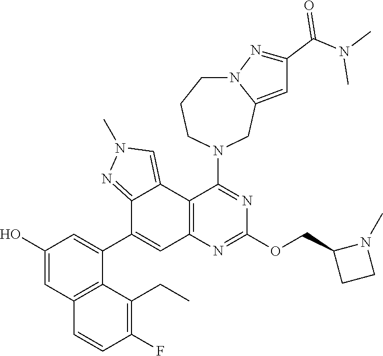 [SMILES: CCc1c(F)ccc2cc(O)cc(-c3cc4nc(OC[C@@H]5CCN5C)nc(N5CCCn6nc(C(=O)N(C)C)cc6C5)c4c4cn(C)nc34)c12] | min, 678.6 [M + H]⁺ (Meth- od 1) | (s, 1H), 7.75 (dd, J = 9.1, 6.1 Hz, 1H), 7.37-7.27 (m, 3H), 7.00 (d, J = 2.5 Hz, 1H), 6.19 (s, 1H), 4.94 (dd, J = 16.0, 4.1 Hz, 1H), 4.79 (dd, J = 16.0, 2.4 Hz, 1H), 4.59-4.45 (m, 2H), 4.31- 4.19 (m, 2H), 4.12- 3.99 (m, 4H), 3.92- 3.81 (m, 1H), 3.30- 3.21 (m, 4H), 2.92 (s, 3H), 2.77-2.68 (m, 1H), 2.44-2.34 (m, 1H), 2.31-2.20 (m, 4H), 2.17-1.84 (m, 4H), 0.56 (t, J = 7.26 Hz, 3H) Exchangeable proton not seen. |
| | Example 70 | | |
| | 5-[4-(8-ethyl-7-fluoro-3- | | |
| | hydroxy-1-naphthyl)-2-methyl- | | |
| | 7-[(2S)-1-methylazetidin-2- | | |
| | yl]methoxy]pyrazolo[4,3- | | |
| | f]quinazolin-9-yl]-N,N-dimethyl- | | |
| | 4,6,7,8-tetrahydropyrazolo[1,5- | | |
| | a][1,4]diazepine-2- | | |
| | carboxamide | | |
| Intermediate 1-62 and | Example 71 | 1.97 | 9.85 (s,1H), 8.47 |
| 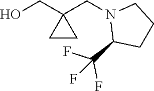 [SMILES: OCC1(CN2CCC[C@H]2C(F)(F)F)CC1] A-19 [1-[(2S)-2- (trifluoromethyl)pyrroli- din-1- yl]methyl]cyclopropyl] methanol (A-19) | 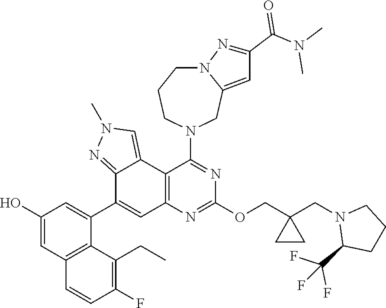 [SMILES: CCc1c(F)ccc2cc(O)cc(-c3cc4nc(OCC5(CN6CCC[C@H]6C(F)(F)F)CC5)nc(N5CCCn6nc(C(=O)N(C)C)cc6C5)c4c4cn(C)nc34)c12] | min, 800.4 [M + H]⁺ (Meth- od 6) | (s, 1H), 7.77-7.72 (m, 1H), 7.34-7.32 (m, 1H), 7.29-7.28 (m, 1H), 7.00 (m, 1H), 6.18-6.17 (m, 1H), 4.94-4.90 (m, 1H), 4.79-7.75 (m, 1H), 4.54-4.49 (m, 3H), 4.07-4.02 (m, 4H), 3.89-3.82 (m, 2H), 3.25 (s, 4H), 3.19-3.16 (m, 3H), 2.92 (s, 3H), 2.41-2.36 (m, 2H), 2.26-2.02 (m, 4H), 1.97-1.89 (m, 1H), 1.77-1.65 (m, 3H), 0.62-0.53 (m, 5H), 0.49-0.45 |
| | Example 71 | | (m, 1H), 0.39-0.36 |
| | 5-[4-(8-ethyl-7-fluoro-3- | | (m, 1H). |
| | hydroxy-1-naphthyl)-2-methyl- | | |
| | 7-[1-[(2S)-2- | | |
| | (trifluoromethyl)pyrrolidin-1- | | |
| | yl]methyl]cyclopropyl]methoxy] | | |
| | pyrazolo[4,3-f]quinazolin-9-yl]- | | |
| | N,N-dimethyl-4,6,7,8- | | |
| | tetrahydropyrazolo[1,5- | | |
| | a][1,4]diazepine-2- | | |
| | carboxamide | | |
| Intermediate I-95 and | Example 94 | 3.33 | 9.80 (s, 1H), 8.46 |
| 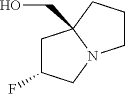 [SMILES: OC[C@@]12CCCN1C[C@H](F)C2] ((2R,7aS)-2- fluorohexahydro-1H- pyrrolizin-7a- yl)methanol | 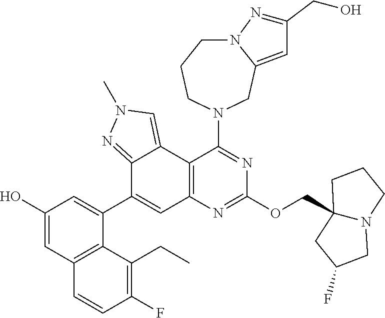 [SMILES: CCc1c(F)ccc2cc(O)cc(-c3cc4nc(OC[C@@]56CCCN5C[C@H](F)C6)nc(N5CCCn6nc(CO)cc6C5)c4c4cn(C)nc34)c12] | min, 695.3 [M + H]⁺ (Meth- od 6) | (s, 1H), 7.75 (dd, J = 9.1, 6.0 Hz, 1H), 7.37-7.27 (m, 3H), 7.00 (d, J = 2.6 Hz, 1H), 5.80 (s, 1H), 5.27 (d, J = 54.2 Hz, 1H), 4.99- 4.83 (m, 2H), 4.72 (d, J = 15.8 Hz, 1H), 4.49- 4.34 (m, 2H), 4.31- 4.22 (m, 2H), 4.07 (s, 3H), 4.05- 3.92 (m, 2H), 3.89- 3.79 (m, 1H), 3.14-2.94 (m, 3H), 2.88-2.75 (m, 1H), 2.30- 2.15 (m, 1H), 2.15- 1.91 (m, 5H), |
| | Example 94 | | 1.88-1.71 (m, |
| | 5-ethyl-6-fluoro-4-[9-[2- | | 4H), 1.31-1.14 |
| | (hydroxymethyl)-4,6,7,8- | | (m, 1H), 0.55 (t, J = |
| | tetrahydropyrazolo[1,5- | | 7.2 Hz, 3H). |
| | a][1,4]diazepin-5-yl]-2-methyl- | | |
| | 7-[[(2R,8S)-2-fluoro- | | |
| | 1,2,3,5,6,7- | | |
| | hexahydropyrrolizin-8- | | |
| | yl]methoxy]pyrazolo[4,3- | | |
| | f]quinazolin-4-yl]naphthalen-2- | | |
| | ol | | |
| Intermediate I-110 and | Example 103 | 3.53 | 9.80 (s, 1H), 8.53 |
| 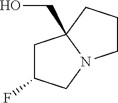 [SMILES: OC[C@@]12CCCN1C[C@H](F)C2] ((2R,7aS)-2- fluorohexahydro-1H- pyrrolizin-7a- yl)methanol | 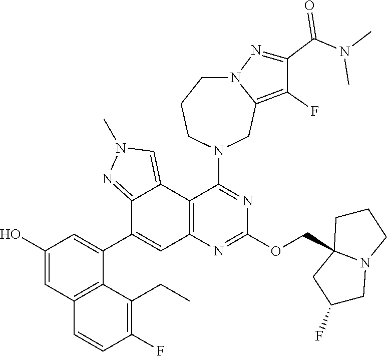 [SMILES: CCc1c(F)ccc2cc(O)cc(-c3cc4nc(OC[C@@]56CCCN5C[C@H](F)C6)nc(N5CCCn6nc(C(=O)N(C)C)c(F)c6C5)c4c4cn(C)nc34)c12] | min, 754.3 [M + H]+ (Meth- od 6) | (s, 1H), 7.75 (dd, J = 6.1, 9.2 Hz, 1H), 7.36 (s, 1H), 7.32 (t, J = 9.4 Hz, 1H), 7.28 (d, J = 2.5 Hz, 1H), 6.98 (d, J = 2.5 Hz, 1H), 5.26 (dbr, J = 55.1 Hz, 1H), 4.89 (d, J = 16.4 Hz, 1H), 4.75 (d, J = 16.2 Hz, 1H), 4.55-4.40 (m, 2H) 4.08 (s, 3H) 4.04 -3.86 (m, 3H), 3.29 (s, 3H), 3.28- 3.11 (m, 3H), 3.10 (s, 3H), 2.81 (mc, 1H), 2.37 (mc, 1H), 2.22-1.62 (m, 10H), 0.54 (t, J = 7.2 Hz, 3H). |
| | Example 103 | | |
| | 5-[4-(8-ethyl-7-fluoro-3- | | |
| | hydroxy-1-naphthyl)-2-methyl- | | |
| | 7-[(2R,8S)-2-fluoro- | | |
| | 1,2,3,5,6,7- | | |
| | hexahydropyrrolizin-8- | | |
| | yl]methoxy]pyrazolo[4,3- | | |
| | f]quinazolin-9-yl]-3-fluoro-N,N- | | |
| | dimethyl-4,6,7,8- | | |
| | tetrahydropyrazolo[1,5- | | |
| | a][1,4]diazepine-2- | | |
| | carboxamide | | |

### Example 105, 4-[4-(8-ethyl-7-fluoro-3-hydroxy-1-naphthyl)-2,5-dimethyl-7-[rac-(2R)-2-fluoro-1,2,3,5,6,7-hexahydropyrrolizin-8-yl]methoxy]pyrazolo[4,3-f]quinazolin-9-yl]-6-methyl-1,4-oxazepan-6-ol

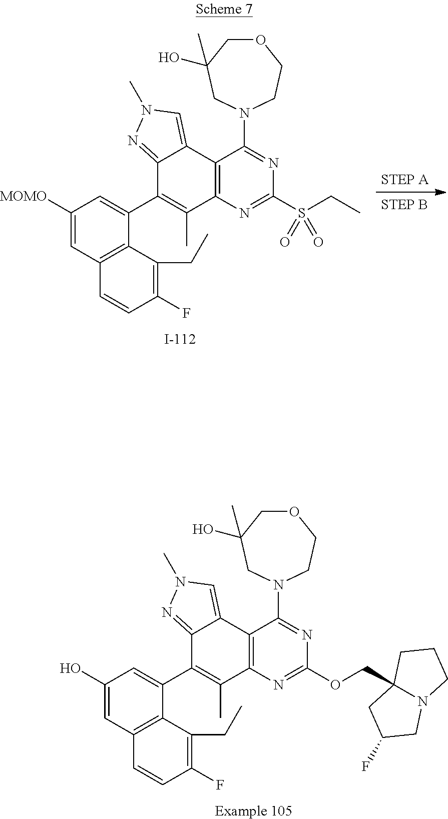

Step A, 4-[4-[8-ethyl-7-fluoro-3-(methoxymethoxy)-1-naphthyl]-2,5-dimethyl-7-[[rac-(2R)-2-fluoro-1,2,3,5,6,7-hexahydropyrrolizin-8-yl]methoxy]pyrazolo[4,3-f]quinazolin-9-yl]-6-methyl-1,4-oxazepan-6-ol. To a solution of ((2R,7aS)-2-Fluorohexahydro-1H-pyrrolizin-7a-yl) methanol (47.63 mg, 0.3 mmol) in THF (0.67 mL) under N₂ was added dropwise 1M Lithium bis(trimethylsilyl)amide in THF (0.28 mL, 0.28 mmol). The reaction mixture was stirred for 20 mins then 4-[4-[8-ethyl-7-fluoro-3-(methoxymethoxy)-1-naphthyl]-7-ethylsulfonyl-2,5-dimethyl-pyrazolo[4,3-f]quinazolin-9-yl]-6-methyl-1,4-oxazepan-6-ol (120. mg, 0.18 mmol) in THF (2.4 mL) was added in one portion. The reaction mixture allowed to warm to room temperature and stirred for 3 hours before being quenched with water and NaHCO₃(aq) and extracted with DCM. The organics were dried over magnesium sulfate, filtered and evaporated to give a brown oil which was purified by flash column chromatography (12 g, eluting in 0-10% MeOH in DCM) to afford t the desired product 4-[4-[8-ethyl-7-fluoro-3-(methoxymethoxy)-1-naphthyl]-2,5-dimethyl-7-[(2R)-2-fluoro-1,2,3,5,6,7-hexahydropyrrolizin-8-yl]methoxy]pyrazolo[4,3-f]quinazolin-9-yl]-6-methyl-1,4-oxazepan-6-ol (20 mg, 0.0279 mmol, 15% yield) as a brown powder.

UPLC-MS (ES⁺, Method 5): 1.85 min, 717.3 [M+H]⁺.

Step B, 4-[4-(8-ethyl-7-fluoro-3-hydroxy-1-naphthyl)-2,5-dimethyl-7-[(2R)-2-fluoro-1,2,3,5,6,7-hexahydropyrrolizin-8-yl]methoxy]pyrazolo[4,3-f]quinazolin-9-yl]-6-methyl-1,4-oxazepan-6-ol. Trifluoroacetic acid (0.21 mL, 2.79 mmol) was added to 4-[4-[8-ethyl-7-fluoro-3-(methoxymethoxy)-1-naphthyl]-2,5-dimethyl-7-[(2R)-2-fluoro-1,2,3,5,6,7-hexahydropyrrolizin-8-yl]methoxy]pyrazolo[4,3-f]quinazolin-9-yl]-6-methyl-1,4-oxazepan-6-ol (20. mg, 0.03 mmol) and the reaction mixture left to stir at room temperature for 2 hours. The mixture was diluted with methanol and the reaction concentrated in vacuo to afford the crude product. Crude material was purified by reverse phase chromatography (12 g, eluting in 5-95% MecN in water (0.1% formic acid)) to afford the desired product 4-[4-(8-ethyl-7-fluoro-3-hydroxy-1-naphthyl)-2,5-dimethyl-7-[[(2R)-2-fluoro-1,2,3,5,6,7-hexahydropyrrolizin-8-yl]methoxy]pyrazolo[4,3-f]quinazolin-9-yl]-6-methyl-1,4-oxazepan-6-ol (1 mg, 0.0015 mmol, 5% yield) as a white powder.

UPLC-MS (ES⁺, Method 1): 3.67 min, 673.3 [M+H]⁺.

The following examples in table 23 were made by analogy with example 105 (scheme 7) starting from the intermediate and building blocks for step A as described in the table.

TABLE 23

| | | UPLC- | |
|---|---|---|---|
| | | MS | ¹H NMR |
| Step A, intermediate + | | (ES⁺): | (400 MHz, |
| building block | Example Structure/Name | min, m/z | DMSO-d₆) δ/ppm |
| Intermediate I-112 and | Example 137 | 1.44 min, | 9.91 (br s, 1H), |
| 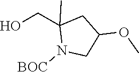 tert-Butyl 2- (hydroxymethyl)-4- methoxy-2- methylpyrrolidine-1- carboxylate | 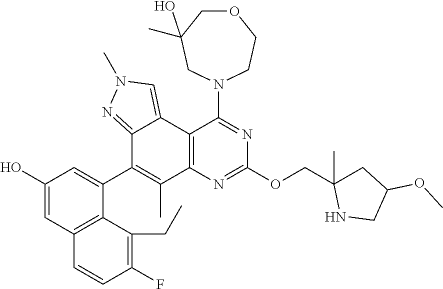 [SMILES: CCc1c(F)ccc2cc(O)cc(-c3c(C)c4nc(OCC5(C)CC(OC)CN5)nc(N5CCOCC(C)(O)C5)c4c4cn(C)nc34)c12] | 659.5 [M + H]+ (Method 1) | 8.43 (s, 1H), 8.32 (s, 1H), 7.77-7.73 (m, 1H), 7.32-7.27 (m, 2H), 6.87-6.85 (m, 1H), 5.63 (br, 1H), 4.37-4.28 (m, 1H), 4.22-4.14 (m, 1H), 4.05(s, 3H), 3.94-3.84 (m, 4H), 3.74-3.68 (m, 3H), 3.58-3.55 (m, 1H), 3.18 (m, 1H), 3.15- 3.14 (m, 3H), 2.94- 2.92 (m, 1H), 2.83- 2.77 (m, 1H), 2.21 (s, 3H), 2.01-1.97 (m, 1H), 1.86-1.82 (m, 1H), 1.75-1.67 |
| | Example 137 | | (m, 1H), 1.26-1.23 |
| | 4-[4-(8-ethyl-7-fluoro-3- | | (m, 2H), 1.19-1.18 |
| | hydroxy-1-naphthyl)-7-[(4- | | (d, 2H), 1.05 (s, |
| | methoxy-2-methyl-pyrrolidin-2- | | 2H), 0.92-0.85 (m, |
| | yl)methoxy]-2,5-dimethyl- | | 1H), 0.43-0.38 (m, |
| | pyrazolo[4,3-f]quinazolin-9-yl]- | | 3H). |
| | 6-methyl-1,4-oxazepan-6-ol | | |
| Intermediate I-112 and | Example 138 | 3.02 min, | 9.76 (s, 1H), |
| 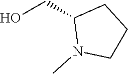 (S)-(−)-1-Methyl-2- pyrrolidinemethanol | 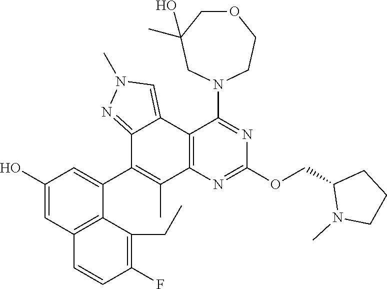 [SMILES: CCc1c(F)ccc2cc(O)cc(-c3c(C)c4nc(OC[C@@H]5CCCN5C)nc(N5CCOCC(C)(O)C5)c4c4cn(C)nc34)c12] | 629.5 [M + H]+ (Method 1) | 8.43(s, 1H), 7.77- 7.73 (m, 1H), 7.32- 7.27 (m, 2H), 6.85 (d, 1H), 5.62-5.58 (m, 1H), 4.46-4.38 (m, 1H), 4.30-4.22 (m, 1H), 4.06 (s, 3H), 3.93-3.83 (m, 3H), 3.73-3.67 (m, 3H), 3.57-3.54 (d, 1H), 3.41 (m, 1H), 3.38 (m, 1H), 2.95 (m, 1H), 2.37-2.36 (m, 3H), 2.21 (s, 3H), 2.19-2.14 (m, 2H), 1.99-1.89 (m, 1H), 1.70-1.63 (m, 3H), 1.23 (s, 1H), |
| | Example 138 | | 1.05 (s, 3H), 0.43- |
| | 4-[4-(8-ethyl-7-fluoro-3- | | 0.39 (t, 3H). |
| | hydroxy-1-naphthyl)-2,5- | | |
| | dimethyl-7-[[(2S)-1- | | |
| | methylpyrrolidin-2- | | |
| | yl]methoxy]pyrazolo[4,3- | | |
| | f]quinazolin-9-yl]-6-methyl-1,4- | | |
| | oxazepan-6-ol | | |
| Intermediate I-112 and | Example 139 | 3.08 min, | 9.79 (br s, 1H), |
| 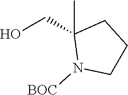 (2S)-1-BOC-2- Methylpyrrolidine-2- methanol | 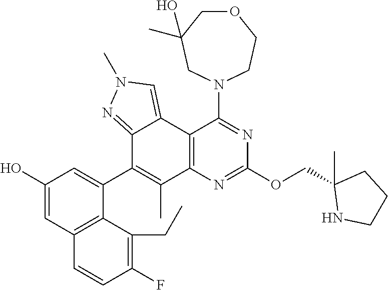 [SMILES: CCc1c(F)ccc2cc(O)cc(-c3c(C)c4nc(OC[C@]5(C)CCCN5)nc(N5CCOCC(C)(O)C5)c4c4cn(C)nc34)c12] | 629.5 [M + H]+ (Method 1) | 8.40 (s, 1H), 8.24 (s, 1H), 7.77-7.73 (m, 1H), 7.33-7.27 (m, 2H), 6.87-6.85 (m, 1H), 5.51 (br s, 1H), 4.36-4.27 (m, 2H), 4.05 (s, 3H), 3.95-3.84 (m, 3H), 3.78-3.69 (m, 3H), 3.58-3.55 (d, J = 12.52, 1H), 3.48- 3.47 (d, J = 7.86, 1H), 3.44 (s, 1H), 3.40 (s, 1H), 3.07- 3.00 (m, 2H), 2.23- 2.22 (m, 3H), 1.90- 1.84 (m, 3H), 1.65- 1.62 (m, 1H), 1.31 |
| | Example 139 | | (s, 3H), 1.04 (s, |
| | 4-[4-(8-ethyl-7-fluoro-3- | | 2H), 0.87 (s, 1H), |
| | hydroxy-1-naphthyl)-2,5- | | 0.42-0.36 (m, 3H). |
| | dimethyl-7-[[(2S)-2- | | |
| | methylpyrrolidin-2- | | |
| | yl]methoxy]pyrazolo[4,3- | | |
| | f]quinazolin-9-yl]-6-methyl-1,4- | | |
| | oxazepan-6-ol | | |
| Intermediate I-112 and | Example 140 | 2.99 min, | 9.78 (br s, 1H), |
| 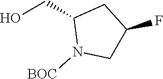 [SMILES: CC(C)(C)OC(=O)N1C[C@H](F)C[C@H]1CO] (2S,4R)-tert-Butyl 4- fluoro-2-(hydroxy- methyl)pyrrolidine- 1-carboxylate | 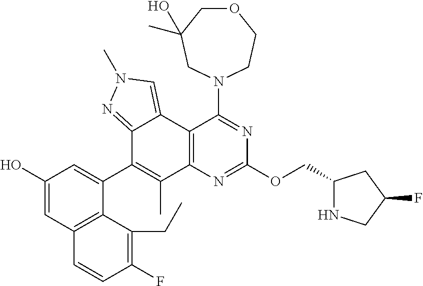 [SMILES: CCc1c(F)ccc2cc(O)cc(-c3c(C)c4nc(OC[C@@H]5C[C@@H](F)CN5)nc(N5CCOCC(C)(O)C5)c4c4cn(C)nc34)c12] | 633.4 [M + H]+ (Method 1) | 8.44 (s, 1H), 8.16 (s, 1H), 7.77-7.73 (m, 1H), 7.32-7.27 (m, 2H), 6.87-6.85 (m, 1H), 5.46 (br s, 1H), 5.34-5.20 (m, 1H), 4.35-4.27 (m, 2H), 4.05 (s, 3H), 3.96-3.83 (m, 3H), 3.77-3.69 (m, 4H), 3.57-3.54 (m, 1H), 3.47 (s, 1H), 3.44- 3.41 (m, 1H), 3.12 (m, 1H), 3.04 (s, 1H), 2.22-2.21 (m, 3H), 2.21-2.09 (m, 2H), 1.84-1.68 (m, 1H), 1.02 (s, 2H), |
| | Example 140 | | 0.86 (s, 1H), 0.42- |
| | 4-[4-(8-ethyl-7-fluoro-3- | | 0.37 (m, 3H). |
| | hydroxy-1-naphthyl)-2,5- | | |
| | dimethyl-7-[[(2S,4R)-4- | | |
| | fluoropyrrolidin-2- | | |
| | yl]methoxy]pyrazolo[4,3- | | |
| | f]quinazolin-9-yl]-6-methyl-1,4- | | |
| | oxazepan-6-ol | | |
| Intermediate I-112 and | Example 141 | 2.97 min, | 9.78-9.75 (d, |
| 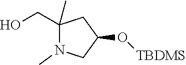 A-17 [(4R)-4-[tert- butyl(dimethyl)silyl] oxy-1,2-dimethyl- pyrrolidin-2- yl]methanol (A-17) | 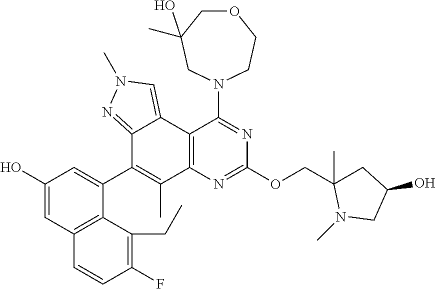 [SMILES: CCc1c(F)ccc2cc(O)cc(-c3c(C)c4nc(OCC5(C)C[C@@H](O)CN5C)nc(N5CCOCC(C)(O)C5)c4c4cn(C)nc34)c12] | 659.7 [M + H]+ (Method 1) | J = 11.4 Hz, 1H), 8.43(s, 1H), 7.77- 7.73(m, 1H), 7.32- 7.27(m, 2H), 6.87- 6.84(m, 1H), 5.83- 5.74(d, J = 33.25, 1H), 4.73(s, 1H), 4.30-4.15(m, 3H), 4.05(s, 3H), 3.92- 3.78(m, 3H), 3.74- 3.49(m, 4H), 3.43- 3.35(m, 1H), 3.09- 3.03(m, 1H), 2.25- 2.19(m, 9H), 1.51- 1.47(m, 1H), 1.23- 0.92(m, 7H), 0.42- 0.35(m, 3H). |
| | Example 141 | | |
| | 4-[4-(8-ethyl-7-fluoro-3- | | |
| | hydroxy-1-naphthyl)-2,5- | | |
| | dimethyl-7-[[(4R)-4-hydroxy- | | |
| | 1,2-dimethyl-pyrrolidin-2- | | |
| | yl]methoxy]pyrazolo[4,3- | | |
| | f]quinazolin-9-yl]-6-methyl-1,4- | | |
| | oxazepan-6-ol | | |
| Intermediate I-112 and | Example 142 | 3.12 min, | 9.78-9.75(d, |
| 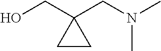 (1- ((dimethylamino) methyl)cyclopropyl) methano | 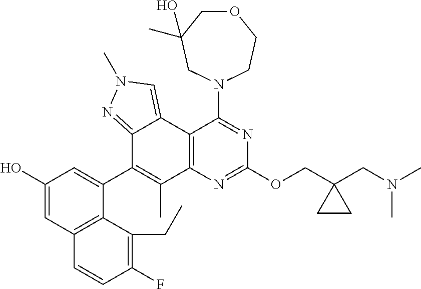 [SMILES: CCc1c(F)ccc2cc(O)cc(-c3c(C)c4nc(OCC5(CN(C)C)CC5)nc(N5CCOCC(C)(O)C5)c4c4cn(C)nc34)c12] | 643.5 [M + H]+ (Method 1) | J = 11.96, 1H), 8.43(s, 1H), 7.77- 7.73(m, 1H), 7.33- 7.27(m, 2H), 6.86- 6.84(m, 1H), 5.64(s, 1H), 4.34- 4.23(m, 2H), 4.04(s, 3H), 3.92- 3.68(m, 6H), 3.58- 3.39(m, 3H), 2.28- 2.26(m, 2H), 2.21- 2.20(d, J = 3.96, 4H), 2.16(s, 5H), 1.23(m, 1H), 1.06(s, 2H), 0.90(m, 1H), 0.64(s, 2H), 0.42- 0.35(m, 5H). |
| | Example 142 | | |
| | 4-[7-[[1-[(dimethyl- | | |
| | amino)methyl]cyclopropyl] | | |
| | methoxy]-4-(8-ethyl-7- | | |
| | fluoro-3-hydroxy-1-naphthyl)- | | |
| | 2,5-dimethyl-pyrazolo[4,3- | | |
| | f]quinazolin-9-yl]-6-methyl-1,4- | | |
| | oxazepan-6-ol | | |
| Intermediate I-112 and | Example 143 | 2.95 min, | 9.75(s, 1H), 8.43(s, |
| 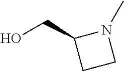 [(2S)-1- methylazetidin-2- yl]methanol | 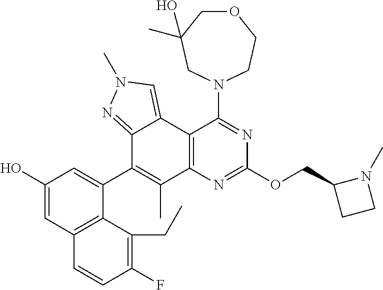 [SMILES: CCc1c(F)ccc2cc(O)cc(-c3c(C)c4nc(OC[C@@H]5CCN5C)nc(N5CCOCC(C)(O)C5)c4c4cn(C)nc34)c12] | 615.5 [M + H]+ (Method 1) | 1H), 7.77-7.73(m, 1H), 7.32-7.27(m, 2H), 6.87-6.85(m, 1H), 5.68-5.59 (d, J = 36.41, 1H), 4.44-4.32(m, 2H), 4.05(s, 3H), 3.93- 3.83(m, 3H), 3.74- 3.60(m, 3H), 3.58- 3.54(d, 1H), 3.42- 3.35(m, 2H), 2.27- 2.26(m, 3H), 2.22- 2.20(m, 3H), 2.00- 1.95(m, 3H), 1.36(s, 1H), 1.26- 1.23 (m, 1H), 1.05- 1.04(m, 3H), 0.87- 0.83(m, 1H), 0.43- |
| | Example 143 | | 0.36(m, 3H). |
| | 4-[4-(8-ethyl-7-fluoro-3- | | |
| | hydroxy-1-naphthyl)-2,5- | | |
| | dimethyl-7-[[(2S)-1- | | |
| | methylazetidin-2- | | |
| | yl]methoxy]pyrazolo[4,3- | | |
| | f]quinazolin-9-yl]-6-methyl-1,4- | | |
| | oxazepan-6-ol | | |
| Intermediate I-112 and | Example 144 | 2.92 min, | 9.77(s, 1H), 8.43(s, |
| 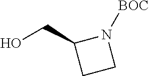 [SMILES: CC(C)(C)OC(=O)N1CC[C@H]1CO] (S)-1-Boc-2- Azetidinemethanol | 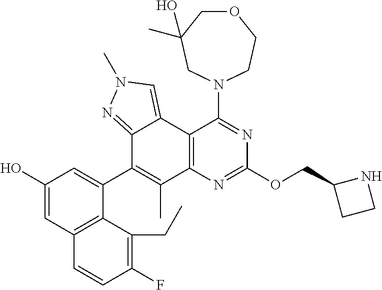 [SMILES: CCc1c(F)ccc2cc(O)cc(-c3c(C)c4nc(OC[C@@H]5CCN5)nc(N5CCOCC(C)(O)C5)c4c4cn(C)nc34)c12] | 601.4 [M + H]+ (Method 1) | 1H), 7.77-7.73(m, 1H), 7.32-7.27(m, 2H), 6.87-6.84(m, 1H), 5.44 (s, 1H), 4.41-4.40(m, 2H), 4.22(br, 1H), 4.05(s, 3H), 3.93- 3.68(m, 7H), 3.57- 3.51(m, 2H), 3.47- 3.41(m, 1H), 2.22- 2.26(m, 3H), 2.22- 2.21(m, 3H), 2.19- 2.11(m, 3H), 1.23 (s, 1H), 1.02(s, 3H), 0.85(m, 1H), 0.42-0.38(m, 3H). |
| | Example 144 | | |
| | 4-[4-(8-ethyl-7-fluoro-3- | | |
| | hydroxy-1-naphthyl)-2,5- | | |
| | dimethyl-7-[[(2S)-azetidin-2- | | |
| | yl]methoxy]pyrazolo[4,3- | | |
| | f]quinazolin-9-yl]-6-methyl-1,4- | | |
| | oxazepan-6-ol | | |
| Intermediate I-112 and | Example 145 | 2.89 min, | 9.78-9.75(m, 1H), |
| 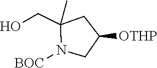 A-37 tert-butyl 2- (hydroxymethyl)-2- methyl-4- tetrahydropyran-2- yloxy-pyrrolidine-1- carboxylate (A-37) | 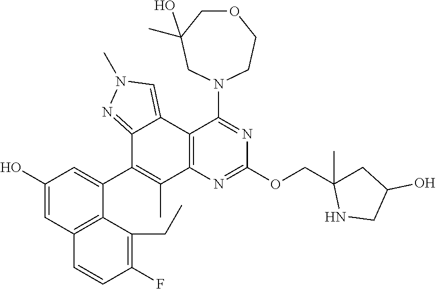 [SMILES: CCc1c(F)ccc2cc(O)cc(-c3c(C)c4nc(OCC5(C)CC(O)CN5)nc(N5CCOCC(C)(O)C5)c4c4cn(C)nc34)c12] | 645.5 [M + H]+ (Method 1) | 8.43(s, 1H), 7.77- 7.73(m, 1H), 7.32- 7.27(m, 2H), 6.87- 6.84(m, 1H), 5.64 (br, 1H), 4.64- 4.59(m, 1H), 4.23- 4.20(m, 1H), 4.11- 4.10(m, 1H), 4.05(s, 3H), 3.93- 3.79(m, 2H), 3.74- 3.67(m, 2H),3.07- 3.00(br, 1H), 2.90- 2.87(m, 1H), 2.23- 2.15(m. 7H), 2.01- 1.94(m, 2H), 1.72- 1.71(m, 1H), 1.48- 1.47(m, 1H), 1.29- 1.28(m, 2H), 1.24- |
| | Example 145 | | 1.23(m, 1H), 1.17- |
| | 4-[4-(8-ethyl-7-fluoro-3- | | 1.16(d, J = 4.00, |
| | hydroxy-1-naphthyl)-7-[(4- | | 1H), 1.06 (s, 2H), |
| | hydroxy-2-methyl-pyrrolidin-2- | | 0.90-0.86(m, 1H), |
| | yl)methoxy]-2,5-dimethyl- | | 0.42-0.35(m, 3H). |
| | pyrazolo[4,3-f]quinazolin-9-yl]- | | |
| | 6-methyl-1,4-oxazepan-6-ol | | |
| Intermediate I-112 and | Example 146 | 3.23 min, | 9.78-9.75(d, |
| 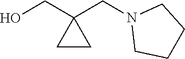 [SMILES: OCC1(CN2CCCC2)CC1] A-11 {1-[(pyrrolidin-1- yl)methyl]cyclopropyl} methanol (A-11) | 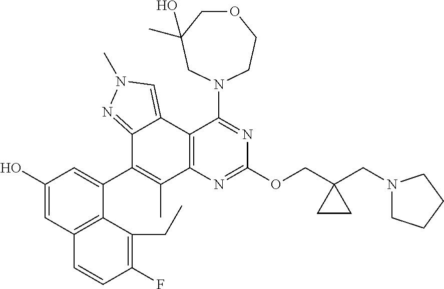 [SMILES: CCc1c(F)ccc2cc(O)cc(-c3c(C)c4nc(OCC5(CN6CCCC6)CC5)nc(N5CCOCC(C)(O)C5)c4c4cn(C)nc34)c12] | 669.5 [M + H]+ (Method 1) | J = 11.28, 1H), 8.42(s, 1H), 7.77- 7.73(q, 1H), 7.32- 7.27(m, 2H), 6.86- 6.84(dd, J = 6.00, 2.88, 1H), 5.64(s, 1H), 4.37-4.29(m, 2H), 4.04(s, 3H), 3.92-3.68(m, 5H), 3.58-3.39(m, 3H), 2.43-2.37(m, 5H), 2.23-2.15(m, 6H), 1.62(s, 4H), 1.05(s, 2H), 0.90(s, 1H), 0.61(s, 2H), 0.42- 0.36(m, 5H). |
| | Example 146 | | |
| | 4-[4-(8-ethyl-7-fluoro-3- | | |
| | hydroxy-1-naphthyl)-2,5- | | |
| | dimethyl-7-[[1-(pyrrolidin-1- | | |
| | ylmethyl)cyclopropyl]methoxy] | | |
| | pyrazolo[4,3-f]quinazolin-9-yl]- | | |
| | 6-methyl-1,4-oxazepan-6-ol | | |
| Intermediate I-112 and | Example 147 | 3.16 min, | 9.78-9.75(d, |
| 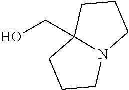 (tetrahydro-1H- pyrrolizin-7a(5H)- yl)methanol | 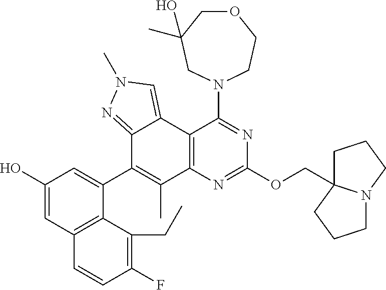 [SMILES: CCc1c(F)ccc2cc(O)cc(-c3c(C)c4nc(OCC56CCCN5CCC6)nc(N5CCOCC(C)(O)C5)c4c4cn(C)nc34)c12] | 655.5 [M + H]+ (Method 1) | J = 10.92, 1H), 8.43(s, 1H), 7.77- 7.73(q, 1H), 7.32- 7.27(m, 2H), 6.87- 6.84(dd, J = 7.00, 2.68, 1H), 5.68 (s, 1H), 4.14-−4.07 (m, 2H), 4.05(s, 3H), 3.92- 3.80(m, 3H), 3.78- 3.67(m, 3H), 3.58- 3.49(m, 1H),3.44- 3.36(m, 1H), 2.95- 2.90(m, 2H), 2.27- 2.13(m, 6H), 1.96- 1.88(m, 2H), 1.84- 1.70(m, 5H), 1.60- 1.52(m, 2H), |
| | Example 147 | | 1.06(s, 2H), 0.91(s, |
| | 4-[4-(8-ethyl-7-fluoro-3- | | 1H), 0.42-0.35(m, |
| | hydroxy-1-naphthyl)-7- | | 3H). |
| | (1,2,3,5,6,7- | | |
| | hexahydropyrrolizin-8- | | |
| | ylmethoxy)-2,5-dimethyl- | | |
| | pyrazolo[4,3-f]quinazolin-9-yl]- | | |
| | 6-methyl-1,4-oxazepan-6-ol | | |
| Intermediate I-112 and | Example 148 | 3.11 min, | 9.78-9.75(d, 1H), |
| 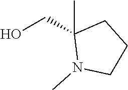 [(2S)-1,2- dimethylpyrrolidin-2- yl]methanol | 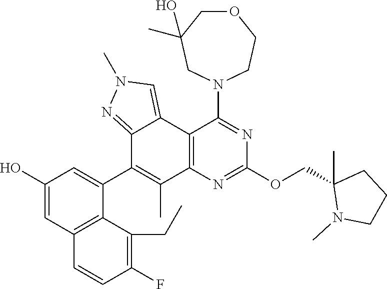 [SMILES: CCc1c(F)ccc2cc(O)cc(-c3c(C)c4nc(OC[C@]5(C)CCCN5C)nc(N5CCOCC(C)(O)C5)c4c4cn(C)nc34)c12] | 643.5 [M + H]+ (Method 1) | 8.42(s, 1H), 7.77- 7.73(m, 1H), 7.33- 7.27(m, 2H), 6.87- 6.85(dd, 1H), 5.81- 5.72(d, 1H), 4.31- 4.21(m, 2H), 4.05(s, 3H), 3.92- 3.84(m, 3H), 3.70- 3.67(m, 3H), 3.58- 3.55 (d, J = , 1H), 2.87-2.81(m, 1H), 2.28-2.26(d, 4H), 2.23-2.17(m, 5H), 1.92(br, 1H), 1.69(br, 1H), 1.58(br, 1H), 1.23 (s, 1H), 1.08- 1.04(m, 5H), |
| | Example 148 | | 0.92(s, 1H), 0.42- |
| | 4-[4-(8-ethyl-7-fluoro-3- | | 0.37(m, 3H). |
| | hydroxy-1-naphthyl)-2,5- | | |
| | dimethyl-7-[[(2S)-1,2- | | |
| | dimethylpyrrolidin-2- | | |
| | yl]methoxy]pyrazolo[4,3- | | |
| | f]quinazolin-9-yl]-6-methyl-1,4- | | |
| | oxazepan-6-ol | | |
| Intermediate I-112 and | Example 149 | 3.14 min, | 9.78-9.75(d, |
| 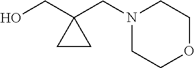 [SMILES: OCC1(CN2CCOCC2)CC1] A-18 [1-(morpholinomethyl) cyclopropyl]methanol (A-18) | 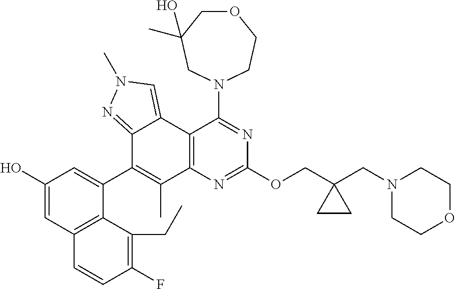 [SMILES: CCc1c(F)ccc2cc(O)cc(-c3c(C)c4nc(OCC5(CN6CCOCC6)CC5)nc(N5CCOCC(C)(O)C5)c4c4cn(C)nc34)c12] | 685.5 [M + H]+ (Method 1) | J = 11.16 Hz, 1H), 8.43(s, 1H), 7.77- 7.73(m, 1H), 7.33- 7.27(m, 2H), 6.86- 6.84(dd, J = 2.76,2.60 Hz, 1H), 5.65(s, 1H), 4.41-4.31(m, 2H), 4.05(s, 3H), 3.93- 3.68(m, 6H), 3.58- 3.42(m, 6H), 2.36- 2.26(m, 8H), 2.22- 2.17(d, J = 4.24 Hz, 3H), 1.05(s, 2H), 0.89(s, 1H), 0.64(s, 2H), 0.42-0.36(m, 5H), |
| | Example 149 | | |
| | 4-[4-(8-ethyl-7-fluoro-3- | | |
| | hydroxy-1-naphthyl)-2,5- | | |
| | dimethyl-7-[[1- | | |
| | (morpholinomethyl)cyclopropyl] | | |
| | methoxy]pyrazolo[4,3- | | |
| | f]quinazolin-9-yl]-6-methyl-1,4- | | |
| | oxazepan-6-ol | | |
| Intermediate I-140 and | Example 150 | 2.92 min, | 9.89-9.81 (m, 1H), |
| 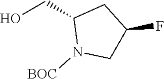 [SMILES: CC(C)(C)OC(=O)N1C[C@H](F)C[C@H]1CO] (2S,4R)-tert-Butyl 4- fluoro-2- (hydroxymethyl) pyrrolidine-1- carboxylate | 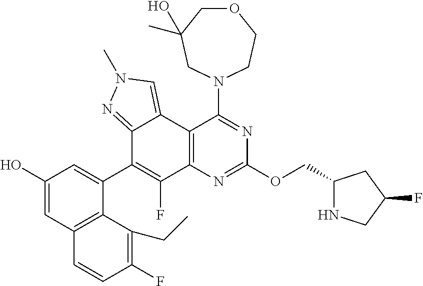 [SMILES: CCc1c(F)ccc2cc(O)cc(-c3c(F)c4nc(OC[C@@H]5C[C@@H](F)CN5)nc(N5CCOCC(C)(O)C5)c4c4cn(C)nc34)c12] | 637.5 [M + H]+ (Method 1) | 8.55-8.49 (m, 1H), 7.81-7.73 (m, 1H), 7.38-7.28 (m, 2H), 7.05-6.99 (m, 1H), 5.35-5.12 (m, 2H), 4.33-4.18 (m, 2H), 4.14-3.83 (m, 7H), 3.83-3.62 (m, 3H), 3.57-3.39 (m, 2H), 3.12-2.94 (m, 2H), 2.45-2.33 (m, 1H), 2.21-2.03 (m, 2H), 1.82-1.61 (m, 1H), 0.99 (s, 2H), 0.81 (s, 1H), 0.57 (t, J = 7.4 Hz, 3H). Exchangeable proton not seen. |
| | Example 150 | | |
| | 4-[4-(8-ethyl-7-fluoro-3- | | |
| | hydroxy-1-naphthyl)-5-fluoro-2- | | |
| | methyl-7-[[(2S,4R)-4- | | |
| | fluoropyrrolidin-2- | | |
| | yl]methoxy]pyrazolo[4,3- | | |
| | f]quinazolin-9-yl]-6-methyl-1,4- | | |
| | oxazepan-6-ol | | |
| Intermediate I-140 and | Example 151 | 2.98 min, | 9.90-9.82 (m, 1H), |
| 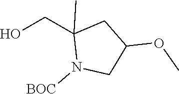 tert-Butyl 2- (hydroxymethyl)-4- methoxy-2- methylpyrrolidine-1- carboxylate | 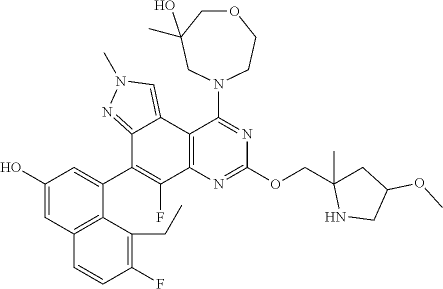 [SMILES: CCc1c(F)ccc2cc(O)cc(-c3c(F)c4nc(OCC5(C)CC(OC)CN5)nc(N5CCOCC(C)(O)C5)c4c4cn(C)nc34)c12] | 663.5 [M + H]+ (Method 1) | 8.55-8.49 (m, 1H), 7.82-7.73 (m, 1H), 7.38-7.28 (m, 2H), 7.04-6.99 (m, 1H), 5.34-5.20 (m, 1H), 4.33-4.03 (m, 5H), 4.02-3.68 (m, 7H), 3.58-3.36 (m, 2H), 3.22-2.76 (m, 5H), 2.48-2.32 (m, 1H), 2.23-1.53 (m, 3H), 1.28-1.14 (m, 3H), 1.02 (s, 2H), 0.83 (s, 1H), 0.57 (t, J = 7.3 Hz, 3H). Exchangeable proton not seen. |
| | Example 151 | | |
| | 4-[4-(8-ethyl-7-fluoro-3- | | |
| | hydroxy-1-naphthyl)-5-fluoro-7- | | |
| | [(4-methoxy-2-methyl- | | |
| | pyrrolidin-2-yl)methoxy]-2- | | |
| | methyl-pyrazolo[4,3- | | |
| | f]quinazolin-9-yl]-6-methyl-1,4- | | |
| | oxazepan-6-ol | | |
| Intermediate I-140 and | Example 152 | 2.86 min, | 9.87-9.85(d, J = |
| 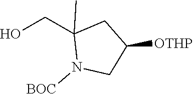 A-37 tert-butyl 2- (hydroxymethyl)-2- methyl-4- tetrahydropyran-2- yloxy-pyrrolidine-1- carboxylate (A-37) | 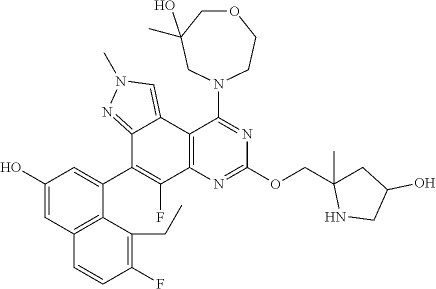 [SMILES: CCc1c(F)ccc2cc(O)cc(-c3c(F)c4nc(OCC5(C)CC(O)CN5)nc(N5CCOCC(C)(O)C5)c4c4cn(C)nc34)c12] | 649.5 [M + H]+ (Method 1) | 6.36, 1H), 8.53- 8.52(d, J = 3.92, 1H), 7.79-7.75(m, 1H), 7.35-7.31(m, 2H), 7.01(s, 1H), 5.28-5.23(m, 1H), 4.78(br, 1H), 4.33- 4.26(m, 2H), 4.14(s, 1H), 4.08(s, 3H), 3.92-3.70(m, 6H), 3.55-3.40(m, 2H), 2.99-2.96(m, 1H), 2.83-2.72(m, 1H), 2.15(br. 2H), 2.04-1.98(m, 1H), 1.77(s, 1H), 1.55- 1.51(m, 1H), 1.34(s, 2H), 1.23(s, |
| | Example 152 | | 1H), 1.00(s, 2H), |
| | 4-[4-(8-ethyl-7-fluoro-3- | | 0.83(s, 1H), 0.58- |
| | hydroxy-1-naphthyl)-5-fluoro-7- | | 0.54(t, J = 7.34, |
| | [(4-hydroxy-2-methyl- | | 3H). |
| | pyrrolidin-2-yl)methoxy]-2- | | |
| | methyl-pyrazolo[4,3- | | |
| | f]quinazolin-9-yl]-6-methyl-1,4- | | |
| | oxazepan-6-ol | | |
| Intermediate I-140 and | Example 153 | 2.89 min, | 9.88-9.86(d, |
| 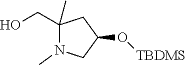 A-17 [(4R)-4-[tert- butyl(dimethyl)silyl] oxy-1,2-dimethyl- pyrrolidin-2- yl]methanol (A-17) | 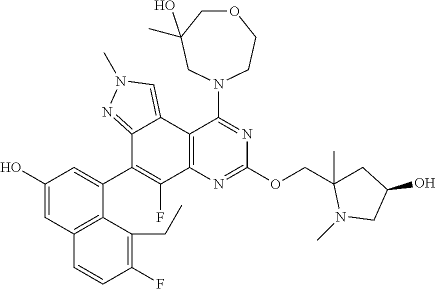 [SMILES: CCc1c(F)ccc2cc(O)cc(-c3c(F)c4nc(OCC5(C)C[C@@H](O)CN5C)nc(N5CCOCC(C)(O)C5)c4c4cn(C)nc34)c12] | 663.5 [M + H]+ (Method 1) | J = 5.92, 1H), 8.55- 8.53(m, 1H), 7.80- 7.60(m, 1H), 7.36- 7.31(m, 2H), 7.01- 7.00(m, 1H), 5.72- 5.20(m, , 2H), 4.81-4.26(m, 4H), 4.08(s, 3H), 3.97- 3.71(m, 7H), 3.58- 3.34(m, 4H), 2.85(br, 1H), 2.15(br, 2H), 1.99- 1.90(m, 1H), 1.76- 1.73(m, 1H), 1.60- 1.38(m, 2H), 1.23(s, 1H), 1.02(s, 2H), 0.85-0.83(m, 1H), 0.58-0.54(t, |
| | Example 153 | | J = 7.42, 3H). |
| | 4-[4-(8-ethyl-7-fluoro-3- | | |
| | hydroxy-1-naphthyl)-5-fluoro-2- | | |
| | methyl-7-[[rac-(4R)-4-hydroxy- | | |
| | 1,2-dimethyl-pyrrolidin-2- | | |
| | yl]methoxy]pyrazolo[4,3- | | |
| | f]quinazolin-9-yl]-6-methyl-1,4- | | |
| | oxazepan-6-ol | | |
| Intermediate I-140 and | Example 154 | 2.95 min, | 9.86-9.85(d, |
| 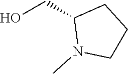 (S)-(−)-1-Methyl-2- pyrrolidinemethanol | 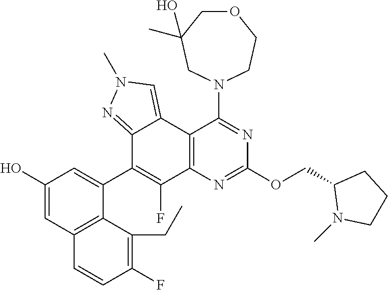 [SMILES: CCc1c(F)ccc2cc(O)cc(-c3c(F)c4nc(OC[C@@H]5CCCN5C)nc(N5CCOCC(C)(O)C5)c4c4cn(C)nc34)c12] | 633.5 [M + H]+ (Method 1) | J = 6.04, 1H), 8.53- 8.52(d, J = 4.28, 1H), 7.79-7.75(m, 1H), 7.35-7.31(m, 2H), 7.02-7.01(d, J = 2.32, 1H), 5.30- 5.22(m, 1H), 4.42- 4.29(m, 2H), 4.08(s, 3H), 3.99- 3.70(m, 7H), 3.55- 3.37(m, 3H), 3.00(br, 1H), 2.43- 2.37(m, 3H), 2.15(br, 2H), 1.98 (br, 1H), 1.70(s, 3H), 1.00(s, 2H), 0.82(s, 1H), 0.59- 0.55(t, J = 7.32, |
| | Example 154 | | 3H). |
| | 4-[4-(8-ethyl-7-fluoro-3- | | |
| | hydroxy-1-naphthyl)-5-fluoro-2- | | |
| | methyl-7-[[(2S)-1- | | |
| | methylpyrrolidin-2- | | |
| | yl]methoxy]pyrazolo[4,3- | | |
| | f]quinazolin-9-yl]-6-methyl-1,4- | | |
| | oxazepan-6-ol | | |
| Intermediate I-140 and | Example 155 | 3.14 min, | 9.87-9.85(d, J = |
| 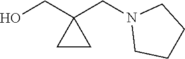 [SMILES: OCC1(CN2CCCC2)CC1] A-11 {1-[(pyrrolidin-1- yl)methyl]cyclopropyl} methanol (A-11) | 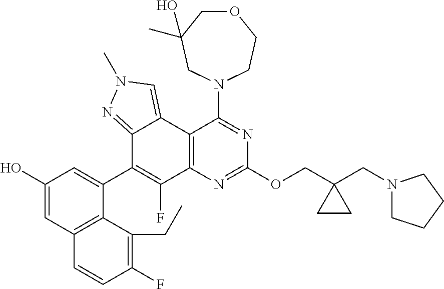 [SMILES: CCc1c(F)ccc2cc(O)cc(-c3c(F)c4nc(OCC5(CN6CCCC6)CC5)nc(N5CCOCC(C)(O)C5)c4c4cn(C)nc34)c12] | 673.6 [M + H]+ (Method 1) | 6.08, 1H), 8.53- 8.52(d, J = 4.24, 1H), 7.80-7.75(m, 1H), 7.36-7.31(m, 2H), 7.01-7.00(m, 1H), 5.32-5.21(d, J = 19.64, 1H), 4.31-4.26(m, 3H), 4.08(s, 3H), 3.88- 3.70(m, 8H), 3.56- 3.40(m, 4H), 3.06(br, 2H), 2.20- 2.12(m, 1H), 1.99- 1.66(m, 5H), 1.38- 1.35(m, 1H), 1.23(s, 1H), 1.02(s, 2H), 0.84(s, 2H), 0.58-0.54(t, |
| | Example 155 | | J = 7.22, 3H). |
| | 4-[4-(8-ethyl-7-fluoro-3- | | |
| | hydroxy-1-naphthyl)-5-fluoro-2- | | |
| | methyl-7-[[1-(pyrrolidin-1- | | |
| | ylmethyl)cyclopropyl]methoxy] | | |
| | pyrazolo[4,3-f]quinazolin-9-yl]- | | |
| | 6-methyl-1,4-oxazepan-6-ol | | |
| Intermediate I-140 and | Example 156 | 2.88 min, | 9.85(br, 1H), 8.55- |
| 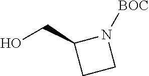 [SMILES: CC(C)(C)OC(=O)N1CC[C@H]1CO] (S)-1-Boc-2- Azetidinemethanol | 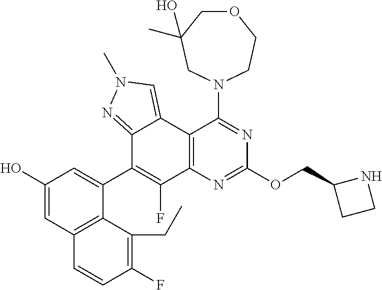 [SMILES: CCc1c(F)ccc2cc(O)cc(-c3c(F)c4nc(OC[C@@H]5CCN5)nc(N5CCOCC(C)(O)C5)c4c4cn(C)nc34)c12] | 605.4 [M + H]+ (Method 1) | 8.53(m, 1H), 7.80- 7.74(m, 1H), 7.36- 7.30(m, 2H), 7.02- 6.99(m, 1H), 5.28 (s, 1H), 5.11(br, 1H), 4.55-4.26(m, 3H), 4.09(s, 3H), 4.04(s, 1H), 3.93- 3.67(m, 8H), 3.09(m, 1H), 2.27- 2.16(m, 2H), 1.91- 1.71(m, 1H), 1.54- 1.47(m, 1H), 1.01- 0.99 (m, 2H), 0.82- 0.79(m, 1H), 0.58- 0.55(m, 3H). |
| | Example 156 | | |
| | 4-[4-(8-ethyl-7-fluoro-3- | | |
| | hydroxy-1-naphthyl)-5-fluoro-2- | | |
| | methyl-7-[[(2S)-azetidin-2- | | |
| | yl]methoxy]pyrazolo[4,3- | | |
| | f]quinazolin-9-yl]-6-methyl-1,4- | | |
| | oxazepan-6-ol | | |
| Intermediate I-140 and | Example 157 | 3.04 min, | 9.86-9.85(d, |
| 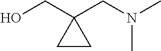 (1-((dimethylamino) methyl)cyclopropyl) methano | 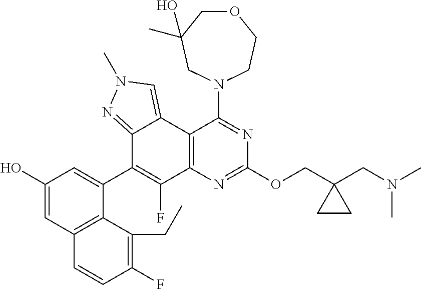 [SMILES: CCc1c(F)ccc2cc(O)cc(-c3c(F)c4nc(OCC5(CN(C)C)CC5)nc(N5CCOCC(C)(O)C5)c4c4cn(C)nc34)c12] | 647.5 [M + H]+ (Method 1) | J = 6.00 Hz, 1H), 8.52-8.51(d, J = 4.36 Hz, 1H), 7.79-7.75(m, 1H), 7.35-7.31(m, 2H), 7.01(d, J = 2.04 Hz, 1H), 5.30(s, 1H), 4.31-4.23(m, 2H), 4.08(s, 3H), 3.98- 3.70(m, 6H), 3.55- 3.35(m, 4H), 2.42- 2.37(m, 1H), 2.16(s, 7H), 1.02(s, 2H), 0.84(s, 1H), 0.66(s, 2H), 0.58- 0.55(t, J = 7.14 Hz, 3H), 0.42(s, 2H). |
| | Example 157 | | |
| | 4-[7-[[1-[(dimethyl- | | |
| | amino)methyl]cyclopropyl] | | |
| | methoxy]-4-(8-ethyl-7- | | |
| | fluoro-3-hydroxy-1-naphthyl)-5- | | |
| | fluoro-2-methyl-pyrazolo[4,3- | | |
| | f]quinazolin-9-yl]-6-methyl-1,4- | | |
| | oxazepan-6-ol | | |
| Intermediate I-140 and | Example 158 | 2.88 min, | 9.86-9.85(d, |
| 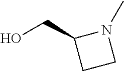 [(2S)-1- methylazetidin-2- yl]methanol | 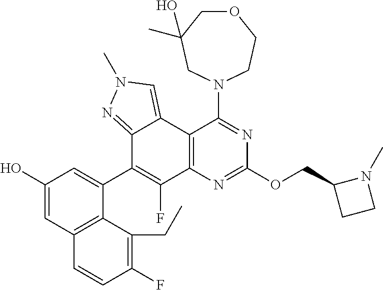 [SMILES: CCc1c(F)ccc2cc(O)cc(-c3c(F)c4nc(OC[C@@H]5CCN5C)nc(N5CCOCC(C)(O)C5)c4c4cn(C)nc34)c12] | 619.5 [M + H]+ (Method 1) | J = 6.04 Hz, 1H), 8.53-8.52(d, J = 4.00 Hz, 1H), 7.79-7.75(m, 1H), 7.35-7.31(m, 2H), 7.02-7.01(m, J = 2.04 Hz, 1H), 5.34-5.21 (m, 1H), 4.43-4.39(m, 2H), 4.08(s, 3H), 3.99- 3.70(m, 6H), 3.55- 3.34(m, 4H), 2.89(s, 1H), 2.44- 2.34(m, 4H), 2.16- 2.04(m, 3H), 1.01- 1.00(d, J = 3.48 Hz, 2H), 0.83-0.82(d, J = 4.24 Hz, 1H), |
| | Example 158 | | 0.59-0.55(t, |
| | 4-[4-(8-ethyl-7-fluoro-3- | | J = 7.38 Hz, 3H). |
| | hydroxy-1-naphthyl)-5-fluoro-2- | | |
| | methyl-7-[[(2S)-1- | | |
| | methylazetidin-2- | | |
| | yl]methoxy]pyrazolo[4,3- | | |
| | f]quinazolin-9-yl]-6-methyl-1,4- | | |
| | oxazepan-6-ol | | |
| Intermediate I-140 and | Example 159 | 2.99 min, | 9.87(br, 1H), 8.53- |
| 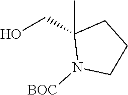 (2S)-1-BOC-2- Methylpyrrolidine-2- methanol | 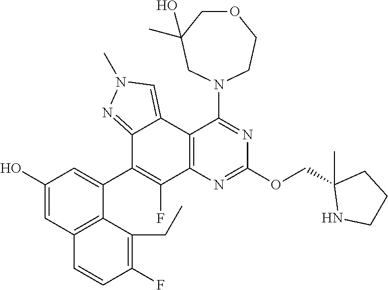 [SMILES: CCc1c(F)ccc2cc(O)cc(-c3c(F)c4nc(OC[C@]5(C)CCCN5)nc(N5CCOCC(C)(O)C5)c4c4cn(C)nc34)c12] | 633.5 [M + H]+ (Method 1) | 8.52(d, J = 4.20 Hz, 1H), 7.79-7.75(m, 1H), 7.35-7.31(m, 2H), 7.02-7.01(d, J = 2.52 Hz, 1H), 5.25-5.23(m, 1H), 4.27-4.19(m, 2H), 4.08(s, 3H), 4.00- 3.71(m, 6H), 3.55- 3.37(m, 3H), 2.99- 2.95 (m, 2H), 2.16(s, 1H), 1.81(m, 3H), 1.59(m, 1H), 1.26- 1.25(m, 3H), 1.01(s, 2H), 0.83(s, 1H), 0.58-0.55(t, J = 7.24 Hz, 3H). |
| | Example 159 | | |
| | 4-[4-(8-ethyl-7-fluoro-3- | | |
| | hydroxy-1-naphthyl)-5-fluoro-2- | | |
| | methyl-7-[[(2S)-2- | | |
| | methylpyrrolidin-2- | | |
| | yl]methoxy]pyrazolo[4,3- | | |
| | f]quinazolin-9-yl]-6-methyl-1,4- | | |
| | oxazepan-6-ol | | |
| Intermediate I-140 and | Example 160 | 3.04 min, | 9.87-9.85(d, |
| 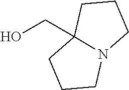 (tetrahydro-1H- pyrrolizin-7a(5H)- yl)methanol | 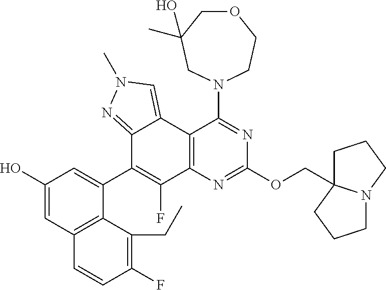 [SMILES: CCc1c(F)ccc2cc(O)cc(-c3c(F)c4nc(OCC56CCCN5CCC6)nc(N5CCOCC(C)(O)C5)c4c4cn(C)nc34)c12] | 659.6 [M + H]+ (Method 1) | J = 6.24 Hz, 1H), 8.52-8.51(d, J = 3.84 Hz, 1H), 7.79-7.75(m, 1H), 7.35-7.31(m, 2H), 7.02-7.01(m, J = 2.04 Hz, 1H), 5.31-5.30 (d, J = 6.68 Hz, 1H), 4.15-4.10(m, 2H), 4.08(s, 3H), 3.98- 3.70(m, 6H), 3.56- 3.42(m, 2H), 3.00(s, 2H), 2.62(s, 1H), 2.20-2.08(m, 1H), 1.96-1.89(m, 3H), 1.86-1.75(m, 5H), 1.63(s, 2H), |
| | Example 160 | | 1.02(s, 2H), 0.84(s, |
| | 4-[4-(8-ethyl-7-fluoro-3- | | 1H), 0.58-0.55(t, |
| | hydroxy-1-naphthyl)-5-fluoro-7- | | J = 7.36 Hz, 3H). |
| | (1,2,3,5,6,7- | | |
| | hexahydropyrrolizin-8- | | |
| | ylmethoxy)-2-methyl- | | |
| | pyrazolo[4,3-f]quinazolin-9-yl]- | | |
| | 6-methyl-1,4-oxazepan-6-ol | | |
| Intermediate I-140 and | Example 161 | 3.00 min, | 9.86-9.85(d, |
| 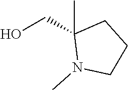 [(2S)-1,2- dimethylpyrrolidin-2- yl]methanol | 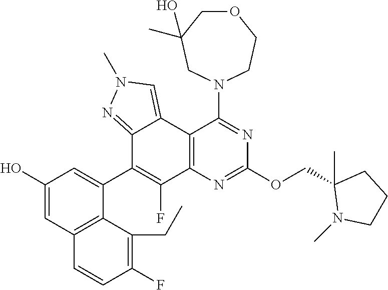 [SMILES: CCc1c(F)ccc2cc(O)cc(-c3c(F)c4nc(OC[C@]5(C)CCCN5C)nc(N5CCOCC(C)(O)C5)c4c4cn(C)nc34)c12] | 647.5 [M + H]+ (Method 1) | J = 5.76 Hz, 1H), 8.52-8.51(d, J = 3.96 Hz, 1H), 7.78-7.75(m, 1H), 7.35-7.31(m, 2H), 7.02-7.01(d, J = 2.48 Hz, 1H), 5.42-5.33(m, 1H), 4.55(s, 2H), 4.31- 4.19(m, 2H), 4.08(s, 3H), 3.97- 3.70(m, 6H), 3.56- 3.37(m, 2H), 2.86(s, 1H), 2.29 (s, 2H), 2.15(s, 2H), 1.93(m, 1H), 1.72-1.69(m, 2H), 1.59(s, 1H), |
| | Example 161 | | 1.05-1.02(m, 5H), |
| | 4-[4-(8-ethyl-7-fluoro-3- | | 0.86-0.85(d, |
| | hydroxy-1-naphthyl)-5-fluoro-2- | | J = 4.12, 1H), 0.58- |
| | methyl-7-[[(2S)-1,2- | | 0.55(t, J = 7.34 Hz, |
| | dimethylpyrrolidin-2- | | 3H). |
| | yl]methoxy]pyrazolo[4,3- | | |
| | f]quinazolin-9-yl]-6-methyl-1,4- | | |
| | oxazepan-6-ol | | |
| Intermediate I-140 and | Example 162 | 3.06 min, | 9.86-9.85(d, |
| 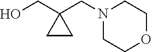 [SMILES: OCC1(CN2CCOCC2)CC1] A-18 [1-(morpholino- methyl)cyclopropyl] methanol (A-18) | 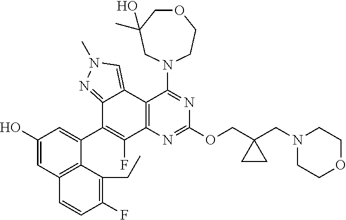 [SMILES: CCc1c(F)ccc2cc(O)cc(-c3c(F)c4nc(OCC5(CN6CCOCC6)CC5)nc(N5CCOCC(C)(O)C5)c4c4cn(C)nc34)c12] | 689.5 [M + H]+ (Method 1) | J = 6.20 Hz, 1H) 8.52-8.51(d, J = 3.24 Hz, 1H), 7.79-7.75(m, 1H), 7.36-7.31(m, 2H), 7.01(m, 1H), 5.30- 5.28(m, 1H), 4.36- 4.29(m, 2H), 4.08(s, 3H), 3.98- 3.70(m, 6H), 3.55- 3.35(m, 8H), 2.37(s, 5H), 2.16(s, 1H), 1.01(s, 2H), 0.84(s, 1H), 0.64(s, |
| | Example 162 | | 2H), 0.59-0.55(t, |
| | 4-[4-(8-ethyl-7-fluoro-3- | | J = 7.80 Hz, 3H), |
| | hydroxy-1-naphthyl)-5-fluoro-2- | | 0.41(s, 2H) |
| | methyl-7-[[1- | | |
| | (morpholinomethyl)cyclopropyl] | | |
| | methoxy]pyrazolo[4,3- | | |
| | f]quinazolin-9-yl]-6-methyl-1,4- | | |
| | oxazepan-6-ol | | |
| Intermediate I-140 and | Example 163 | 3.75 min, | 9.99-9.86 (m, 1H), |
| 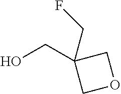 (3- (fluoromethyl)oxetan- 3-yl)methanol | 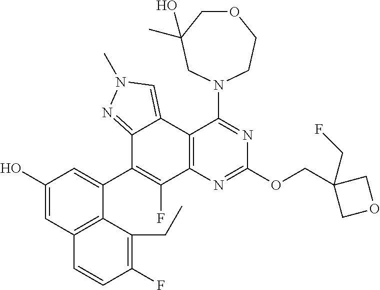 [SMILES: CCc1c(F)ccc2cc(O)cc(-c3c(F)c4nc(OCC5(CF)COC5)nc(N5CCOCC(C)(O)C5)c4c4cn(C)nc34)c12] | 638.6 [M + H]+ (Method 1) | 8.57-8.51 (m, 1H), 7.82-7.73 (m, 1H), 7.38-7.28 (m, 2H), 7.02 (d, J = 2.6 Hz, 1H), 5.16-5.07 (m, 1H), 4.75 (d, J = 47.2 Hz, 2H), 4.68- 4.60 (m, 2H), 4.60- 4.53 (m, 2H), 4.52- 4.43 (m, 2H), 4.18- 3.70 (m, 9H), 3.57- 3.40 (m, 2H), 2.46- 2.34 (m, 1H), 2.22- 2.08 (m, 1H), 0.99 (s, 2H), 0.81 (s, 1H), 0.62-0.51 (m, 3H). |
| | Example 163 | | |
| | 4-[4-(8-ethyl-7-fluoro-3- | | |
| | hydroxy-1-naphthyl)-5-fluoro-7- | | |
| | [[3-(fluoromethyl)oxetan-3- | | |
| | yl]methoxy]-2-methyl- | | |
| | pyrazolo[4,3-f]quinazolin-9-yl]- | | |
| | 6-methyl-1,4-oxazepan-6-ol | | |
| Intermediate I-140 and | Example 164 | 3.80 min, | 9.94-9.86 (m, 1H), |
| 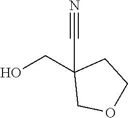 3- (hydroxymethyl) oxolane-3-carbonitrile | 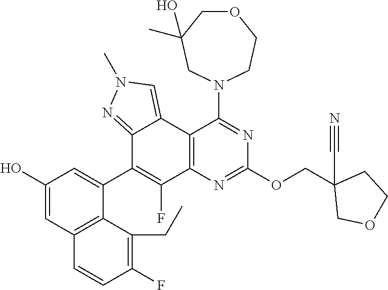 [SMILES: CCc1c(F)ccc2cc(O)cc(-c3c(F)c4nc(OCC5(C#N)CCOC5)nc(N5CCOCC(C)(O)C5)c4c4cn(C)nc34)c12] | 645.5 [M + H]+ (Method 1) | 8.58-8.52 (m, 1H), 7.82-7.73 (m, 1H), 7.38-7.29 (m, 2H), 7.02 (d, J = 2.5 Hz, 1H), 5.10-4.97 (m, 1H), 4.67-4.50 (m, 2H), 4.19-3.86 (m, 11H), 3.86-3.70 (m, 2H), 3.57-3.40 (m, 2H), 2.53-2.33 (m, 2H), 2.33-2.08 (m, 2H), 0.98 (s, 2H), 0.79 (s, 1H), 0.62-0.51 (m, 3H) |
| | Example 164 | | |
| | 3-[[4-(8-ethyl-7-fluoro-3- | | |
| | hydroxy-1-naphthyl)-5-fluoro-9- | | |
| | (6-hydroxy-6-methyl-1,4- | | |
| | oxazepan-4-yl)-2-methyl- | | |
| | pyrazolo[4,3-f]quinazolin-7- | | |
| | yl]oxymethyl]tetrahydrofuran-3- | | |
| | carbonitrile | | |
| Intermediate I-140 and | Example 165 | 3.76 min, | 9.94-9.87 (m, 1H), |
| 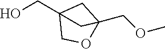 A-31 [1-(methoxymethyl)-2- oxabicyclo[2.1.1] hexan-4-yl]methanol | 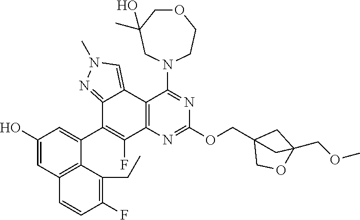 [SMILES: CCc1c(F)ccc2cc(O)cc(-c3c(F)c4nc(OCC56COC(COC)(C5)C6)nc(N5CCOCC(C)(O)C5)c4c4cn(C)nc34)c12] | 676.6 [M + H]+ (Method 1) | 8.56-8.50 (m, 1H), 7.81-7.74 (m, 1H), 7.38-7.29 (m, 2H), 7.02 (d, J = 2.4 Hz, 1H), 5.28-5.16 (m, 1H), 4.7504.61 (m, 2H), 4.08 (s, 3H), 4.02-3.64 (m, 8H), 3.57-3.39 (m, 4H), 3.28-3.26 (m, 3H), 2.45-2.32 (m, 1H), 2.22-2.07 (m, 1H), 1.83-1.75 (m, 2H), 1.56-1.50 (m, 2H), |
| | Example 165 | | 1.00 (s, 2H), 0.82 |
| | 4-[4-(8-ethyl-7-fluoro-3- | | (s, 1H), 0.61-0.53 |
| | hydroxy-1-naphthyl)-5-fluoro-7- | | (m, 3H). |
| | [[1-(methoxymethyl)-2- | | |
| | oxabicyclo[2.1.1]hexan-4- | | |
| | yl]methoxy]-2-methyl- | | |
| | pyrazolo[4,3-f]quinazolin-9-yl]- | | |
| | 6-methyl-1,4-oxazepan-6-ol | | |
| Intermediate I-140 and | Example 166 | 3.14 min, | 9.86-9.85(d, |
| 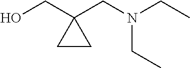 A-32 [1-(diethylaminomethyl) cyclopropyl]methanol | 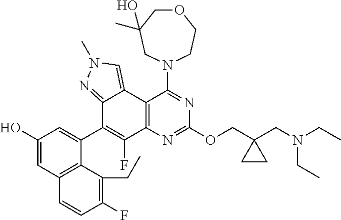 [SMILES: CCc1c(F)ccc2cc(O)cc(-c3c(F)c4nc(OCC5(CN(CC)CC)CC5)nc(N5CCOCC(C)(O)C5)c4c4cn(C)nc34)c12] | 675.6 [M + H]+ (Method 1) | J = 5.80 Hz, 1H), 8.51-8.50(d, J = 3.29 Hz, 1H), 7.79-7.75(m, 1H), 7.35-7.31(m, 2H), 7.02-7.01(t, J = 2.08 Hz, 1H), 5.38-5.28 (d, J = 40.89 Hz, 1H), 4.36-4.21(m, 2H), 4.07(s, 3H), 3.95-3.70(m, 6H), 3.54-3.37(m, 6H), 2.42-2.36(m, 3H), 2.15(s, 1H), 1.01(s, |
| | Example 166 | | 1H), 0.90-0.83(m, |
| | -[7-[[1-(diethyl- | | 8H), 0.61-0.54(m, |
| | aminomethyl)cyclopropyl] | | 5H), 0.40(s, 2H), |
| | methoxy]-4-[8-ethyl-7-fluoro- | | |
| | 3-(methoxymethoxy)-1- | | |
| | naphthyl]-5-fluoro-2-methyl- | | |
| | pyrazolo[4,3-f]quinazolin-9-yl]- | | |
| | 6-methyl-1,4-oxazepan-6-ol | | |
| Intermediate I-142 and | Example 167 | 3.01 min, | 9.85 (s, 1H), 8.41 |
| 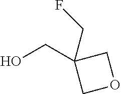 (3- (fluoromethyl)oxetan- 3-yl)methanol | 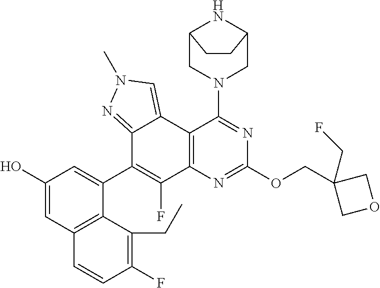 [SMILES: CCc1c(F)ccc2cc(O)cc(-c3c(F)c4nc(OCC5(CF)COC5)nc(N5CC6CCC(C5)N6)c4c4cn(C)nc34)c12] | 619.4 [M + H]+ (Method 1) | (s, 1H), 7.77 (dd, J = 9.2, 6.0 Hz, 1H), 7.37-7.29 (m, 2H), 7.01 (d, J = 2.6 Hz, 1H), 4.76 (d, J = 47.3 Hz, 2H), 4.67- 4.54 (m, 4H), 4.51- 4.45 (m, 2H), 4.18- 4.09 (m, 1H), 4.06 (s, 3H), 3.95-3.84 (m, 1H), 3.65-3.56 (m, 1H), 3.56-3.46 (m, 3H), 2.45-2.34 (m, 1H), 2.21-2.06 (m, 1H), 1.80-1.67 (m, 1H), 1.65-1.51 (m, 3H), 0.56 (t, J = 7.4 Hz, 3H). |
| | Example 167 | | Exchangeable |
| | 4-[9-(3,8- | | proton not seen. |
| | diazabicyclo[3.2.1]octan-3-yl)- | | |
| | 5-fluoro-7-[[3- | | |
| | (fluoromethyl)oxetan-3- | | |
| | yl]methoxy]-2-methyl- | | |
| | pyrazolo[4,3-f]quinazolin-4-yl]- | | |
| | 5-ethyl-6-fluoro-naphthalen-2-ol | | |
| Intermediate I-142 and | Example 168 | 3.04 min, | 9.86 (s, 1H), 8.42 |
| 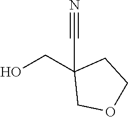 3- (hydroxymethyl) oxolane-3-carbonitrile | 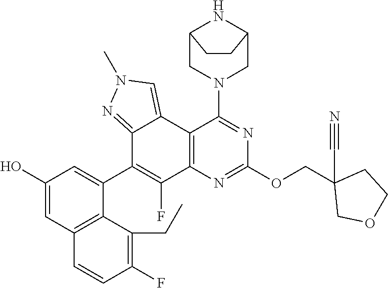 [SMILES: CCc1c(F)ccc2cc(O)cc(-c3c(F)c4nc(OCC5(C#N)CCOC5)nc(N5CC6CCC(C5)N6)c4c4cn(C)nc34)c12] | 626.7 [M + H]+ (Method 1) | (s, 1H), 7.77 (dd, J = 9.1, 6.1 Hz, 1H), 7.38-7.29 (m, 2H), 7.01 (d, J = 2.6 Hz, 1H), 4.65-4.46 (m, 2H), 4.20-4.02 (m, 5H), 3.96-3.82 (m, 4H), 3.66-3.56 (m, 1H), 3.56-3.43 (m, 3H), 2.56-2.35 (m, 2H), 2.31-2.21 (m, 1H), 2.21-2.08 (m, 1H), 1.79-1.49 (m, 4H), 0.56 (t, J = 7.4 Hz, 3H). Exchangeable proton not seen. |
| | Example 168 | | |
| | 3-[[9-(3,8- | | |
| | diazabicyclo[3.2.1]octan-3-yl)- | | |
| | 4-(8-ethyl-7-fluoro-3-hydroxy- | | |
| | 1-naphthyl)-5-fluoro-2-methyl- | | |
| | pyrazolo[4,3-f]quinazolin-7- | | |
| | yl]oxymethyl]tetrahydrofuran-3- | | |
| | carbonitrile | | |
| Intermediate I-142 and | Example 169 | 3.03 min, | 9.85 (s, 1H), 8.39 |
| 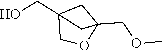 A-31 [1-(methoxymethyl)-2- oxabicyclo[2.1.1] hexan-4-yl]methanol | 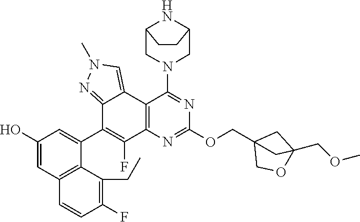 [SMILES: CCc1c(F)ccc2cc(O)cc(-c3c(F)c4nc(OCC56COC(COC)(C5)C6)nc(N5CC6CCC(C5)N6)c4c4cn(C)nc34)c12] | 657.6 [M + H]+ (Method 1) | (s, 1H), 7.77 (dd, J = 9.1, 6.1 Hz, 1H), 7.37-7.28 (m, 2H), 7.01 (d, J = 2.6 Hz, 1H), 4.72-4.59 (m, 2H), 4.13-4.02 (m, 4H), 3.94-3.85 (m, 1H), 3.67 (s, 2H), 3.61-3.43 (m, 6H), 3.28 (s, 3H), 2.44- 2.33 (m, 1H), 2.19- 2.07 (m, 1H), 1.82- 1.75 (m, 2H), 1.75- 1.50 (m, 6H), 0.55 |
| | Example 169 | | (t, J = 7.3 Hz, 3H). |
| | 4-[9-(3,8- | | Exchangeable |
| | diazabicyclo[3.2.1]octan-3-yl)- | | proton not seen. |
| | 5-fluoro-7-[[1-(methoxymethyl)- | | |
| | 2-oxabicyclo[2.1.1]hexan-4- | | |
| | yl]methoxy]-2-methyl- | | |
| | pyrazolo[4,3-f]quinazolin-4-yl]- | | |
| | 5-ethyl-6-fluoro-naphthalen-2-ol | | |
| Intermediate I-145 and | Example 174 | 3.07 min, | 9.86 (s, 1H), 8.50 |
| 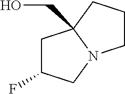 [SMILES: OC[C@@]12CCCN1C[C@H](F)C2] ((2R,7aS)-2- fluorohexahydro-1H- pyrrolizin-7a- yl)methanol | 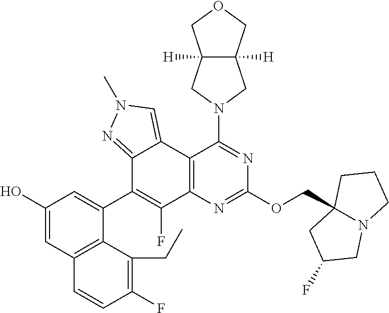 [SMILES: CCc1c(F)ccc2cc(O)cc(-c3c(F)c4nc(OC[C@@]56CCCN5C[C@H](F)C6)nc(N5C[C@H]6COC[C@H]6C5)c4c4cn(C)nc34)c12] | 659.7 [M + H]+ (Method 1) | (s, 1H), 7.77 (dd, J = 9.0, 6.1 Hz, 1H), 7.39-7.28 (m, 2H), 7.01 (d, J = 2.4 Hz, 1H), 5.40-5.18 (m, 1H), 4.21-3.94 (m, 6H), 3.94-3.83 (m, 1H), 3.83-3.61 (m, 4H), 3.49-3.37 (m, 1H), 3.37-3.27 (m, 1H), 3.19-2.95 (m, 5H), 2.90-2.77 (m, 1H), 2.47-2.33 (m, 1H), 2.25-1.94 (m, 4H), 1.92-1.71 (m, 3H), 0.56 (t, J = 7.2 Hz, 3H) |
| | Example 174 | | |
| | 5-ethyl-6-fluoro-4-[5-fluoro-2- | | |
| | methyl-9-[(3aR,6aS)- | | |
| | 1,3,3a,4,6,6a- | | |
| | hexahydrofuro[3,4-c]pyrrol-5- | | |
| | yl]-7-[[(2R,8S)-2-fluoro- | | |
| | 1,2,3,5,6,7- | | |
| | hexahydropyrrolizin-8- | | |
| | yl]methoxy]pyrazolo[4,3-f] | | |
| | quinazolin-4-yl]naphthalen-2-ol | | |
| Intermediate I-146 and | Example 175 | 3.01 min, | 9.87-9.82 (m, 1H), |
| 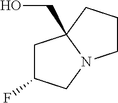 [SMILES: OC[C@@]12CCCN1C[C@H](F)C2] ((2R,7aS)-2- fluorohexahydro-1H- pyrrolizin-7a- yl)methanol | 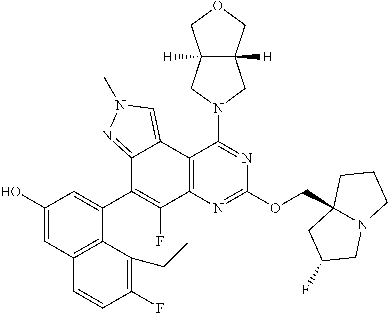 [SMILES: CCc1c(F)ccc2cc(O)cc(-c3c(F)c4nc(OC[C@@]56CCCN5C[C@H](F)C6)nc(N5C[C@H]6COC[C@@H]6C5)c4c4cn(C)nc34)c12] | 659.6 [M + H]+ (Method 1) | 8.58-8.49 (m, 1H), 7.81-7.73 (m, 1H), 7.37-7.28 (m, 2H), 7.03-6.97 (m, 1H), 5.39-5.16 (m, 1H), 4.22-3.82 (m, 9H), 3.55-3.32 (m, 4H), 3.16-2.96 (m, 3H), 2.88-2.78 (m, 1H), 2.62-2.45 (m, 2H), 2.43-2.31 (m, 1H), 2.20-1.95 (m, 4H), 1.91-1.70 (m, 3H), 0.63-0.51 (m, 3H). |
| | Example 175 | | |
| | 5-ethyl-6-fluoro-4-[5-fluoro-2- | | |
| | methyl-9-[(3aS,6aS)- | | |
| | 1,3,3a,4,6,6a- | | |
| | hexahydrofuro[3,4-c]pyrrol-5- | | |
| | yl]-7-[[(2R,8S)-2-fluoro- | | |
| | 1,2,3,5,6,7- | | |
| | hexahydropyrrolizin-8- | | |
| | yl]methoxy]pyrazolo[4,3-f] | | |
| | quinazolin-4-yl]naphthalen-2-ol | | |
| Intermediate I-148 and | Example 176 | 3.19 min, | 9.87 (d, J = 1.2 Hz, |
| 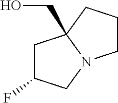 [SMILES: OC[C@@]12CCCN1C[C@H](F)C2] ((2R,7aS)-2- fluorohexahydro-1H- pyrrolizin-7a- yl)methanol | 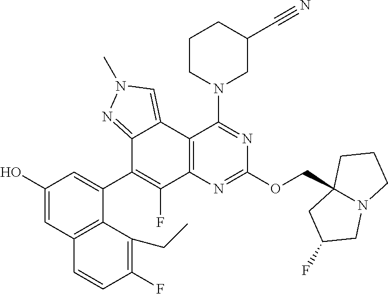 [SMILES: CCc1c(F)ccc2cc(O)cc(-c3c(F)c4nc(OC[C@@]56CCCN5C[C@H](F)C6)nc(N5CCCC(C#N)C5)c4c4cn(C)nc34)c12] | 656.6 [M + H]+ (Method 1) | 1H), 8.48 (s, 1H), 7.78 (dd, J = 9.1, 6.1 Hz, 1H), 7.40- 7.29 (m, 2H), 7.05- 6.98 (m, 1H), 5.41- 5.19 (m, 1H), 4.24- 4.02 (m, 4H), 3.92- 3.81 (m, 1H), 3.73- 3.28 (m, 4H), 3.19- 2.95 (m, 3H), 2.92- 2.77 (m, 1H), 2.63- 2.35 (m, 2H), 2.26- 1.70 (m, 11H), 0.63-0.50 (m, 3H). |
| | Example 176 | | |
| | 1-[4-(8-ethyl-7-fluoro-3- | | |
| | hydroxy-1-naphthyl)-5-fluoro-2- | | |
| | methyl-7-[[(2R,8S)-2-fluoro- | | |
| | 1,2,3,5,6,7- | | |
| | hexahydropyrrolizin-8- | | |
| | yl]methoxy]pyrazolo[4,3-f] | | |
| | quinazolin-9-yl]piperidine-3- | | |
| | carbonitrile | | |
| Intermediate I-150 and | Example 177 | 2.98 min, | 9.86 (s, 1H), 8.46 |
| 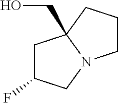 [SMILES: OC[C@@]12CCCN1C[C@H](F)C2] ((2R,7aS)-2- fluorohexahydro-1H- pyrrolizin-7a- yl)methanol | 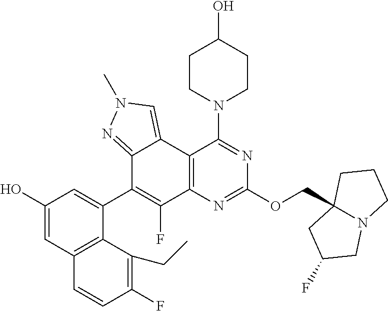 [SMILES: CCc1c(F)ccc2cc(O)cc(-c3c(F)c4nc(OC[C@@]56CCCN5C[C@H](F)C6)nc(N5CCC(O)CC5)c4c4cn(C)nc34)c12] | 647.7 [M + H]+ (Method 1) | (s, 1H), 7.77 (dd, J = 9.1, 6.1 Hz, 1H), 7.39-7.28 (m, 2H), 7.01 (d, J = 2.6 Hz, 1H), 5.41-5.19 (m, 1H), 4.85 (d, J = 3.5 Hz, 1H), 4.23- 4.00 (m, 5H), 3.88- 3.71 (m, 3H), 3.35- 3.18 (m, 2H), 3.18- 2.98 (m, 3H), 2.89- 2.78 (m, 1H), 2.47- 2.32 (m, 1H), 2.26- 1.61 (m, 11H), 0.56 (t, J = 7.2 Hz, 3H). |
| | Example 177 | | |
| | 1-[4-(8-ethyl-7-fluoro-3- | | |
| | hydroxy-1-naphthyl)-5-fluoro-2- | | |
| | methyl-7-[[(2R,8S)-2-fluoro- | | |
| | 1,2,3,5,6,7- | | |
| | hexahydropyrrolizin-8- | | |
| | yl]methoxy]pyrazolo[4,3- | | |
| | f]quinazolin-9-yl]piperidin-4-ol | | |
| Intermediate I-152 and | Example 178 | 2.98 min, | 9.87-9.84 (m, 1H), |
| 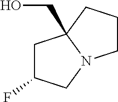 [SMILES: OC[C@@]12CCCN1C[C@H](F)C2] ((2R,7aS)-2- fluorohexahydro-1H- pyrrolizin-7a- yl)methanol | 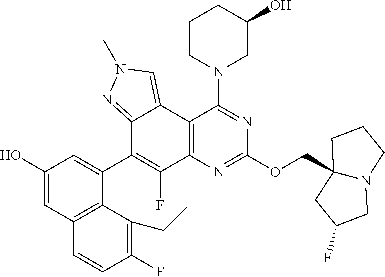 [SMILES: CCc1c(F)ccc2cc(O)cc(-c3c(F)c4nc(OC[C@@]56CCCN5C[C@H](F)C6)nc(N5CCC[C@@H](O)C5)c4c4cn(C)nc34)c12] | 647.7 [M + H]+ (Method 1) | 8.57-8.51 (m, 1H), 7.78 (dd, J = 9.2, 6.1 Hz, 1H), 7.38- 7.30 (m, 2H), 7.00 (d, J = 2.6 Hz, 1H), 5.41-5.18 (m, 1H), 5.03-4.95 (m, 1H), 4.21-4.01 (m, 5H), 3.89-3.75 (m, 2H), 3.74-3.60 (m, 1H), 3.27-2.93 (m, 5H), 2.89-2.76 (m, 1H), 2.47-2.35 (m, 1H), 2.26-1.67 (m, 10H), 1.55-1.41 (m, 1H), 0.57 (t, J = 7.3 Hz, 3H). |
| | Example 178 | | |
| | (3R)-1-[4-(8-ethyl-7-fluoro-3- | | |
| | hydroxy-1-naphthyl)-5-fluoro-2- | | |
| | methyl-7-[[(2R,8S)-2-fluoro- | | |
| | 1,2,3,5,6,7- | | |
| | hexahydropyrrolizin-8- | | |
| | yl]methoxy]pyrazolo[4,3- | | |
| | f]quinazolin-9-yl]piperidin-3-ol | | |
| Intermediate I-154 and | Example 179 | 2.48 min, | 9.85 (1H, br s), |
| 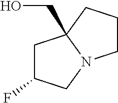 [SMILES: OC[C@@]12CCCN1C[C@H](F)C2] ((2R,7aS)-2- fluorohexahydro-1H- pyrrolizin-7a- yl)methanol | 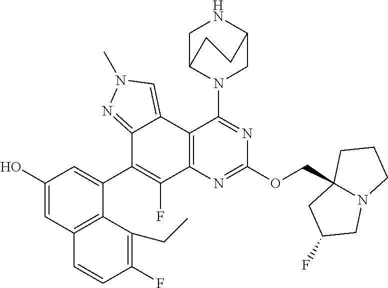 [SMILES: CCc1c(F)ccc2cc(O)cc(-c3c(F)c4nc(OC[C@@]56CCCN5C[C@H](F)C6)nc(N5CC6CCC5CN6)c4c4cn(C)nc34)c12] | 658.5 [M + H]+ (Method 1) | 8.42 (1H, d, J = 4.3 Hz), 7.77 (1H, dd, J = 9.1, 6.1 Hz), 7.36-7.30 (2H, m), 7.02-6.99 (1H, m), 5.37-5.19 (1H, m), 4.41-4.35 (1H, m), 4.17-3.98 (5H, m), 3.82-3.47 (2H, m), 3.17-2.98 (5H, m), 2.87-2.78 (1H, m), 2.45-2.35 (2H, m), 2.22-1.70 (11H, m), 0.55 (3H, t, J = 7.3 Hz). Exchangeable proton not seen. |
| | Example 179 | | |
| | 4-[9-(2,5- | | |
| | diazabicyclo[2.2.2]octan-2-yl)- | | |
| | 5-fluoro-2-methyl-7-[[(2R,8S)- | | |
| | 2-fluoro-1,2,3,5,6,7- | | |
| | hexahydropyrrolizin-8- | | |
| | yl]methoxy]pyrazolo[4,3- | | |
| | f]quinazolin-4-yl]-5-ethyl-6- | | |
| | fluoro-naphthalen-2-ol | | |
| Intermediate I-160 and | Example 182 | 3.27 min, | 9.77(s, 1H), 8.44(s, |
| 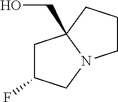 [SMILES: OC[C@@]12CCCN1C[C@H](F)C2] ((2R,7aS)-2- fluorohexahydro-1H- pyrrolizin-7a- yl)methanol | 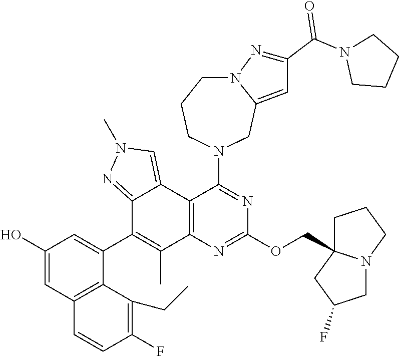 [SMILES: CCc1c(F)ccc2cc(O)cc(-c3c(C)c4nc(OC[C@@]56CCCN5C[C@H](F)C6)nc(N5CCCn6nc(C(=O)N7CCCC7)cc6C5)c4c4cn(C)nc34)c12] | 776.7 [M + H]+ (Method 1) | 1H), 7.77-7.74(m, 1H), 7.33-7.28(dd, J = 9.72, 2.84 Hz, 2H), 6.87-6.86(d, J = 2.48 Hz, 1H), 6.23(s, 1H), 5.32- 5.18 (d, J = 55.89 Hz, 1H), 4.95-4.76(dd, J = 60.87, 15.86 Hz, 2H), 4.52(s, 2H), 4.11-4.07(m, 1H), 4.05(s, 3H), 3.99- 3.96(m, 1H), 3.87- 3.76(m, 4H), 3.42- 3.39(t, J = 6.82 Hz, 2H), 3.07-3.04(m, 2H), 2.98(s, 1H), 2.81(s, 1H), 2.19(s, 3H), 2.12-1.99(m, 6H), 1.88-1.74(m, 8H), 0.45-0.41(t, |
| | Example 182 | | J = 7.38 Hz, 3H). |
| | [5-[4-(8-ethyl-7-fluoro-3- | | |
| | hydroxy-1-naphthyl)-7- | | |
| | [[(2R,8S)-2-fluoro-1,2,3,5,6,7- | | |
| | hexahydropyrrolizin-8- | | |
| | yl]methoxy]-2,5-dimethyl- | | |
| | pyrazolo[4,3-f]quinazolin-9-yl]- | | |
| | 4,6,7,8-tetrahydropyrazolo[1,5- | | |
| | a][1,4]diazepin-2-yl]-pyrrolidin- | | |
| | 1-yl-methanone | | |
| Intermediate I-140 and | Example 187 | 3.17 min, | 9.87-9.84(m, 1H), |
| 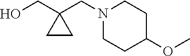 A-33 [1-[(4-methoxy-1- piperidyl)methyl] cyclopropyl]methanol (A-33) | 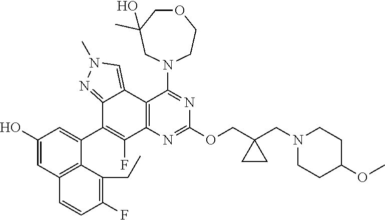 [SMILES: CCc1c(F)ccc2cc(O)cc(-c3c(F)c4nc(OCC5(CN6CCC(OC)CC6)CC5)nc(N5CCOCC(C)(O)C5)c4c4cn(C)nc34)c12] | 717.6 [M + H]+ (Method 1) | 8.51-8.51(d, J = 3.24 Hz, 1H), 7.79-7.75(m, 1H), 7.35-7.30(m, 2H), 7.01-6.99(m, 1H), 5.33-5.27(m, 1H), 4.35-4.26(m, 2H), 4.08(s, 3H), 3.94- 3.69(m, 6H), 3.55- 3.40(m, 3H), 3.18(s, 3H), 3.09(s, 1H), 2.72(s, 1H), 2.30-2.27(m, 1H), 2.16(s, 3H), 2.04- |
| | Example 187 | | 1.97(m, 2H), |
| | 4-[4-(8-ethyl-7-fluoro-3- | | 1.75(m, 2H), 1.37- |
| | hydroxy-1-naphthyl)-5-fluoro-7- | | 1.33(m, 1H), |
| | [[1-[(4-methoxy-1- | | 1.23(m, 3H), |
| | piperidyl)methyl]cyclopropyl] | | 1.01(s, 1H), 0.85- |
| | methoxy]-2-methyl-pyrazolo[4,3- | | 0.83(m, 2H), |
| | f]quinazolin-9-yl]-6-methyl-1,4- | | 0.64(s, 1H), 0.58- |
| | oxazepan-6-ol | | 0.55(t, J = 7.34 Hz, |
| | | | 3H), 0.39(m, 1H), |
| Intermediate I-140 and | Example 188 | 3.61 min, | 9.86-9.84(d, |
| 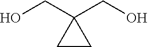 [1- (Hydroxymethyl) cyclopropyl]methanol | 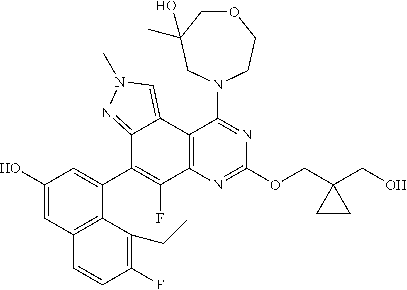 [SMILES: CCc1c(F)ccc2cc(O)cc(-c3c(F)c4nc(OCC5(CO)CC5)nc(N5CCOCC(C)(O)C5)c4c4cn(C)nc34)c12] | 620.6 [M + H]+ (Method 1) | J = 6.28 Hz, 1H), 8.52-8.51(d, J = 3.84 Hz, 1H), 7.79-7.75(m, 1H), 7.35-7.31(m, 2H), 7.02-7.01(d, J = 2.64 Hz, 1H), 5.25(s, 1H), 4.65- 4.61(m, 1H),4.33- 4.26(m, 2H), 4.08(s, 3H), 3.99- 3.70(m, 6H), 3.56- 3.36(m, 5H), 2.15(s, 1H), 1.00(s, 2H), 0.82(m, 1H), 0.58-0.55(m, 5H), 0.51-0.48(m, 2H), |
| | Example 188 | | |
| | 4-[4-(8-ethyl-7-fluoro-3- | | |
| | hydroxy-1-naphthyl)-5-fluoro-7- | | |
| | [[1- | | |
| | (hydroxymethyl)cyclopropyl] | | |
| | methoxy]-2-methyl-pyrazolo[4,3- | | |
| | f]quinazolin-9-yl]-6-methyl-1,4- | | |
| | oxazepan-6-ol | | |
| Intermediate I-167 and | Example 195 | 3.05 min, | 9.89 (s, 1H), 8.56 |
| 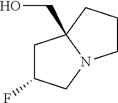 [SMILES: OC[C@@]12CCCN1C[C@H](F)C2] ((2R,7aS)-2- fluorohexahydro-1H- pyrrolizin-7a- yl)methanol | 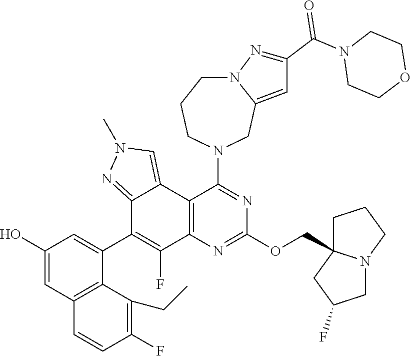 [SMILES: CCc1c(F)ccc2cc(O)cc(-c3c(F)c4nc(OC[C@@]56CCCN5C[C@H](F)C6)nc(N5CCCn6nc(C(=O)N7CCOCC7)cc6C5)c4c4cn(C)nc34)c12] | 796.5 [M + H]+ (Method 1) | (s, 1H), 7.78 (dd, J = 6.1, 9.0 Hz, 1H), 7.39-7.30 (m, 2H), 7.03 (d, J = 2.3 Hz, 1H), 6.36 (br s, 1H), 5.35 (d, J = 53.1 Hz, 1H), 4.97 (d, J = 16.5 Hz, 1H), 4.85 (d, J = 15.9 Hz, 1H), 4.60-4.41 (m, 2H), 4.09 (s, 3H), 4.20-3.86 (m, 6H), 3.70-3.50 (m, 6H), 3.25-2.81 (m, 3H), 2.49-2.37 (m, 1H), 2.27- 1.72 (m, 9H), 1.32- 1.14 (m, 1H), 0.59 (t, J = 7.3 Hz, 3H) ppm. |
| | Example 195 | | |
| | [5-[4-(8-ethyl-7-fluoro-3- | | |
| | hydroxy-1-naphthyl)-5-fluoro-7- | | |
| | [[(2R,8S)-2-fluoro-1,2,3,5,6,7- | | |
| | hexahydropyrrolizin-8- | | |
| | yl]methoxy]-2-methyl- | | |
| | pyrazolo[4,3-f]quinazolin-9-yl]- | | |
| | 4,6,7,8-tetrahydropyrazolo[1,5- | | |
| | a][1,4]diazepin-2-yl]- | | |
| | morpholino-methanone | | |
| Intermediate I-169 and | Example 196 | 3.08 min, | 9.86 (s, 1H), 8.54 |
| 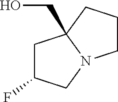 [SMILES: OC[C@@]12CCCN1C[C@H](F)C2] ((2R,7aS)-2- fluorohexahydro-1H- pyrrolizin-7a- yl)methanol | 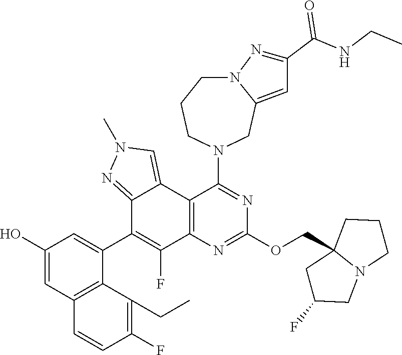 [SMILES: CCNC(=O)c1cc2n(n1)CCCN(c1nc(OC[C@@]34CCCN3C[C@H](F)C4)nc3c(F)c(-c4cc(O)cc5ccc(F)c(CC)c45)c4nn(C)cc4c13)C2] | 754.6 [M + H]+ (Method 1) | (s, 1H), 8.01 (t, J = 6.0 Hz, 1H), 7.78 (dd, J = 6.1, 9.1 Hz, 1H), 7.34 (t, J = 9.4 Hz, 1H), 7.33 (d, J = 2.8 Hz, 1H), 7.03 (d, J = 2.5 Hz, 1H), 6.31 (d, J = 2.5 Hz, 1H), 5.26 (d, J = 55.2 Hz, 1H), 4.96 (d, J = 16.4 Hz, 1H), 4.83 (d, J = 15.9 Hz, 1H), 4.60- 4.40 (m, 2H), 4.08 (s, 3H), 4.07-3.85 (m, 4H), 3.19 (m, 2H), 3.12-2.96 (m, 3H), 2.80 (mc, 1H), 2.23-1.65 (m, 10H), 1.05 (t, J = 7.1 Hz, 3H), 0.59 (t, J = 7.3 Hz, 3H). |
| | Example 196 | | |
| | N-ethyl-5-[4-(8-ethyl-7-fluoro- | | |
| | 3-hydroxy-1-naphthyl)-5-fluoro- | | |
| | 7-[[(2R,8S)-2-fluoro- | | |
| | 1,2,3,5,6,7- | | |
| | hexahydropyrrolizin-8- | | |
| | yl]methoxy]-2-methyl- | | |
| | pyrazolo[4,3-f]quinazolin-9-yl]- | | |
| | 4,6,7,8-tetrahydropyrazolo[1,5- | | |
| | a][1,4]diazepine-2- | | |
| | carboxamide | | |
| Intermediate I-171 and | Example 197 | 3.15 min, | 9.87 (s, 1H), 8.54 |
| 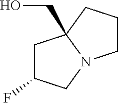 [SMILES: OC[C@@]12CCCN1C[C@H](F)C2] ((2R,7aS)-2- fluorohexahydro-1H- pyrrolizin-7a- yl)methanol | 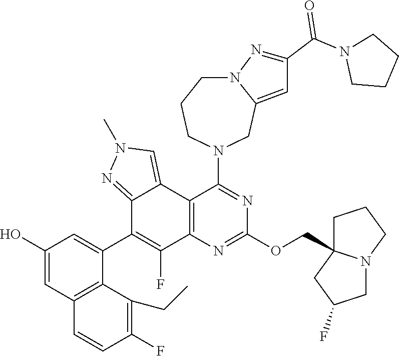 [SMILES: CCc1c(F)ccc2cc(O)cc(-c3c(F)c4nc(OC[C@@]56CCCN5C[C@H](F)C6)nc(N5CCCn6nc(C(=O)N7CCCC7)cc6C5)c4c4cn(C)nc34)c12] | 781.0 [M + H]+ (Method 1) | (s, 1H), 7.78 (dd, J = 6.0, 9.2 Hz, 1H), 7.34 (t, J = 9.3 Hz, 1H), 7.33 (d, J = 2.8 Hz, 1H), 7.03 (d, J = 2.6 Hz, 1H), 6.32 (s, 1H), 5.23 (d, J = 53.3 Hz, 1H), 4.97 (d, J = 16.3 Hz, 1H), 4.83 (d, J = 15.9 Hz, 1H), 4.59-4.45 (m, 2H), 4.08 (s, 3H), 4.06-3.85 (m, 4H), 3.80 (m, 2H), 3.42 (t, J = 6.7 Hz, 2H), 3.12-2.92 (m, 3H), 2.81 (m, 1H), 2.26- 1.67 (m, 14H), 0.59 (t, J = 7.3 Hz, 3H) |
| | Example 197 | | |
| | [5-[4-(8-ethyl-7-fluoro-3- | | |
| | hydroxy-1-naphthyl)-5-fluoro-7- | | |
| | [[(2R,8S)-2-fluoro-1,2,3,5,6,7- | | |
| | hexahydropyrrolizin-8- | | |
| | yl]methoxy]-2-methyl- | | |
| | pyrazolo[4,3-f]quinazolin-9-yl]- | | |
| | 4,6,7,8-tetrahydropyrazolo[1,5- | | |
| | a][1,4]diazepin-2-yl]-pyrrolidin- | | |
| | 1-yl-methanone | | |
| Intermediate I-173 and | Example 198 | 3.07 min, | 9.77 (1H, s), 8.47 |
| 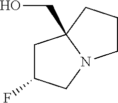 [SMILES: OC[C@@]12CCCN1C[C@H](F)C2] ((2R,7aS)-2- fluorohexahydro-1H- pyrrolizin-7a- yl)methanol | 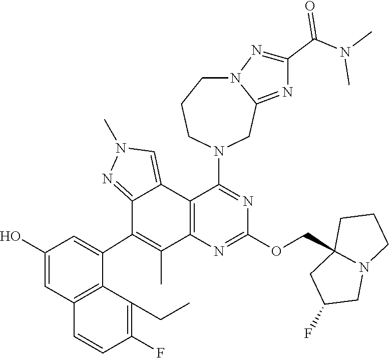 [SMILES: CCc1c(F)ccc2cc(O)cc(-c3c(C)c4nc(OC[C@@]56CCCN5C[C@H](F)C6)nc(N5CCCn6nc(C(=O)N(C)C)nc6C5)c4c4cn(C)nc34)c12] | 751.9 [M + H]+ (Method 1) | (1H, s), 7.75 (1H, dd, J = 9.1, 6.1 Hz), 7.34-7.26 (2H, m), 6.86 (1H, d, J = 2.6 Hz), 5.41-5.16 (1H, m), 5.00-4.88 (2H, m), 4.42-4.32 (2H, m), 4.05 (3H, s), 4.05-3.89 (3H, m) 3.24-2.74 (10H, m), 2.31- 1.59 (14H, m), 0.40 (3H, t, J = 7.3 Hz). |
| | Example 198 | | |
| | 8-[4-(8-ethyl-7-fluoro-3- | | |
| | hydroxy-1-naphthyl)-2,5- | | |
| | dimethyl-7-[[rac-(2R,8S)-2- | | |
| | fluoro-1,2,3,5,6,7- | | |
| | hexahydropyrrolizin-8- | | |
| | yl]methoxy]pyrazolo[4,3- | | |
| | f]quinazolin-9-yl]-N,N-dimethyl- | | |
| | 5,6,7,9-tetrahydro- | | |
| | [1,2,4]triazolo[1,5- | | |
| | a][1,4]diazepine-2- | | |
| | carboxamide | | |
| Intermediate I-175 and | Example 201 | 3.07 min, | 9.80 (s, 1H), 8.26 |
| 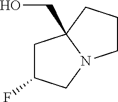 [SMILES: OC[C@@]12CCCN1C[C@H](F)C2] ((2R,7aS)-2- fluorohexahydro-1H- pyrrolizin-7a- yl)methanol | 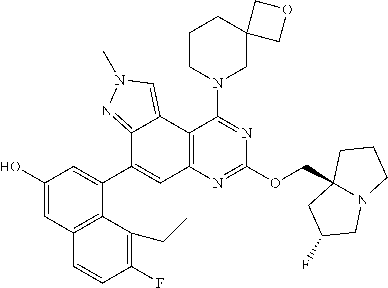 [SMILES: CCc1c(F)ccc2cc(O)cc(-c3cc4nc(OC[C@@]56CCCN5C[C@H](F)C6)nc(N5CCCC6(COC6)C5)c4c4cn(C)nc34)c12] | 655.3 [M + H]+ (Method 1) | (s, 1H), 7.75 (dd, J = 9.6, 6.3 Hz, 1H), 7.37 (s, 1H), 7.27- 7.37 (m, 2H), 6.98 (d, J = 2.7 Hz, 1H), 5.29 (d, J = 54.0 HZ, 1H), 4.35- 4.41 (m, 2H), 4.29- 4.34 (m, 1H), 4.23- 4.28 (m, 1H), 4.12- 4.17 (m, 1H), 4.11 (s, 3 H), 4.03-4.08 (m, 1H), 3.60-3.82 (m, 2H), 3.05-3.14 (m, 5H), 1.94-2.12 (m, 6H), 1.73-1.91 (m, 7H), 0.50-0.56 (m, 3H). |
| | Example 201 | | |
| | 5-ethyl-6-fluoro-4-[2-methyl-9- | | |
| | (2-oxa-8-azaspiro[3.5]nonan-8- | | |
| | yl)-7-[[(2R,8S)-2-fluoro- | | |
| | 1,2,3,5,6,7- | | |
| | hexahydropyrrolizin-8- | | |
| | yl]methoxy]pyrazolo[4,3-f] | | |
| | quinazolin-4-yl]naphthalen-2-ol | | |
| Intermediate I-177 and | Example 202 | 3.21 min, | 9.85 (d, J = 1.9 Hz, |
| 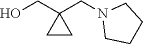 [SMILES: OCC1(CN2CCCC2)CC1] A-11 {1-[(pyrrolidin-1- yl)methyl]cyclopropyl} methanol (A-11) | 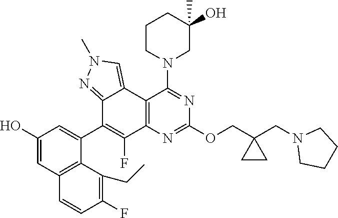 [SMILES: CCc1c(F)ccc2cc(O)cc(-c3c(F)c4nc(OCC5(CN6CCCC6)CC5)nc(N5CCC[C@@](C)(O)C5)c4c4cn(C)nc34)c12] | 657.7 [M + H]+ (Method 1) | 1H), 8.69-8.57 (m, 1H), 7.79-7.75 (m, 1H), 7.36-7.30 (m, 2H), 7.01 (d, J = 2.6 Hz, 1H), 4.62-4.58 (m, 1H), 4.29 (s, 2H), 4.08 (s, 3H), 3.66-3.22 (m, 6H), 2.46-2.36 (m, 5H), 2.22-2.03 (m, 2H), 1.80-1.59 (m, 7H), 1.11-1.00 (m, 3H), 0.63 (br, s, 2H), 0.59-0.52 (m, 3H), |
| | Example 202 | | 0.42 (br, s, 2H). |
| | (3R)-1-[4-(8-ethyl-7-fluoro-3- | | |
| | hydroxy-1-naphthyl)-5-fluoro-2- | | |
| | methyl-7-[[1-(pyrrolidin-1- | | |
| | ylmethyl)cyclopropyl]methoxy] | | |
| | pyrazolo[4,3-f]quinazolin-9-yl]- | | |
| | 3-methyl-piperidin-3-ol | | |
| Intermediate I-177 and | Example 203 | 3.10 min, | 9.86 (d, J = 1.7 Hz, |
| 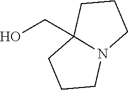 (tetrahydro-1H- pyrrolizin-7a(5H)- yl)methanol | 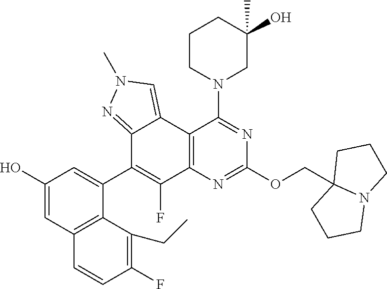 [SMILES: CCc1c(F)ccc2cc(O)cc(-c3c(F)c4nc(OCC56CCCN5CCC6)nc(N5CCC[C@@](C)(O)C5)c4c4cn(C)nc34)c12] | 643.8 [M + H]+ (Method 1) | 1H), 8.68-8.55 (m, 1H), 7.80-7.75 (m, 1H), 7.36-7.30 (m, 2H), 7.02 (d, J = 2.8 Hz, 1H), 4.63-4.55 (m, 1H), 4.12-4.04 (m, 5H), 3.66-3.21 (m, 4H), 2.98-2.91 (m, 2H), 2.59-2.37 (m, 2H), 2.21-2.06 (m, 2H), 1.96-1.55 (m, 12H), 1.11- 0.99 (m, 3H), 0.60- 0.53 (m, 3H). |
| | Example 203 | | |
| | (3R)-1-[4-(8-ethyl-7-fluoro-3- | | |
| | hydroxy-1-naphthyl)-5-fluoro-7- | | |
| | (1,2,3,5,6,7- | | |
| | hexahydropyrrolizin-8- | | |
| | ylmethoxy)-2-methyl- | | |
| | pyrazolo[4,3-f]quinazolin-9-yl]- | | |
| | 3-methyl-piperidin-3-ol | | |
| Intermediate I-178 and | Example 204 | 3.14 min, | 9.77 (d, J = 2.0 Hz, |
| 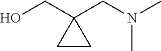 (1-((dimethylamino) methyl)cyclopropyl) methano | 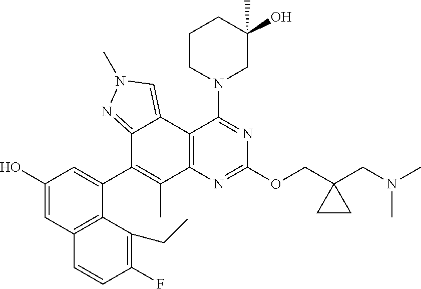 [SMILES: CCc1c(F)ccc2cc(O)cc(-c3c(C)c4nc(OCC5(CN(C)C)CC5)nc(N5CCC[C@@](C)(O)C5)c4c4cn(C)nc34)c12] | 627.5 [M + H]+ (Method 1) | 1H), 8.54 (br s, 1H), 7.75 (dd, J = 9.2, 6.1 Hz, 1H), 7.25-7.34 (m, 2H), 6.85 (d, J = 2.1 Hz, 1H), 4.62-4.70 (m, 1H), 4.24-4.35 (m, 2H), 4.06 (s, 3H), 3.38-3.48 (m, 3H), 1.93-2.40 (m, 14H), 1.53-1.86 (m, 4H), 1.13 (s, 1H), 1.05 (s, 2H), 0.67 (s, 2H), 0.31- 0.52 (m, 5H). |
| | Example 204 | | |
| | (3R)-1-[7-[[1-[(dimethyl- | | |
| | amino)methyl]cyclopropyl] | | |
| | methoxy]-4-(8-ethyl-7- | | |
| | fluoro-3-hydroxy-1-naphthyl)- | | |
| | 2,5-dimethyl-pyrazolo[4,3- | | |
| | f]quinazolin-9-yl]-3-methyl- | | |
| | piperidin-3-ol | | |
| Intermediate I-178 and | Example 205 | 3.15 min, | 9.78 (br s, 1H), |
| 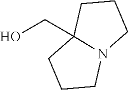 (tetrahydro-1H- pyrrolizin-7a(5H)- yl)methanol | 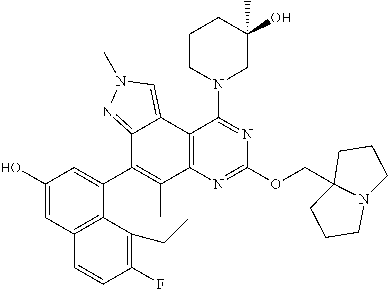 [SMILES: CCc1c(F)ccc2cc(O)cc(-c3c(C)c4nc(OCC56CCCN5CCC6)nc(N5CCC[C@@](C)(O)C5)c4c4cn(C)nc34)c12] | 639.3 [M + H]+ (Method 1) | 8.53 (br s, 1H), 7.75 (d, J = 9.1, 6.2 Hz, 1H), 7.24-7.25 (m, 2H), 6.83 (s, 1H), 4.64 (s, br, 1H), 4.26 (s, br, 2H), 4.06 (s, 3H), 3.38-3.60 (m, 3H), 3.05-3.24 (m, 3H), 2.80 (s, br, 2H), 2.22 (d, J = 4.6 Hz, 3H), 1.56-2.10 (m, 14H), 1.12 (s, 1H), 1.04 (s, 2H), 0.32- 0.44 (m, 3H). |
| | Example 205 | | |
| | (3R)-1-[4-(8-ethyl-7-fluoro-3- | | |
| | hydroxy-1-naphthyl)-7- | | |
| | (1,2,3,5,6,7- | | |
| | hexahydropyrrolizin-8- | | |
| | ylmethoxy)-2,5-dimethyl- | | |
| | pyrazolo[4,3-f]quinazolin-9-yl]- | | |
| | 3-methyl-piperidin-3-ol | | |
| Intermediate I-180 and | Example 206 | 3.72 min, | 9.88 (s, 1H), 8.56 |
| 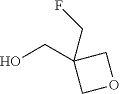 (3- (fluoromethyl)oxetan- 3-yl)methanol | 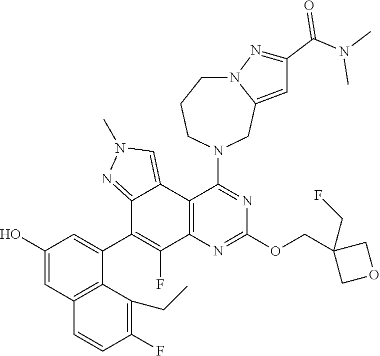 [SMILES: CCc1c(F)ccc2cc(O)cc(-c3c(F)c4nc(OCC5(CF)COC5)nc(N5CCCn6nc(C(=O)N(C)C)cc6C5)c4c4cn(C)nc34)c12] | 715.7 [M + H]+ (Method 1) | (s, 1H), 7.78 (dd, J = 9.0, 6.0 Hz, 1H), 7.39-7.30 (m, 2H), 7.03 (d, J = 2.5 Hz, 1H), 6.33 (s, 1H), 4.97 (d, J = 16.1 Hz, 1H), 4.85 (d, J = 15.7 Hz, 1H), 4.71 (d, J = 47.2 Hz, 2H), 4.59-4.43 (m, 8H), 4.13-4.05 (m, 4H), 3.99-3.87 (m, 1H), 3.25 (s, 3H), 2.93 (s, 3H), 2.48-2.37 (m, 1H), 2.26-2.07 (m, 3H), 0.59 (t, J = 7.3 Hz, 3H) |
| | Example 206 | | |
| | 5-[4-(8-ethyl-7-fluoro-3- | | |
| | hydroxy-1-naphthyl)-5-fluoro-7- | | |
| | [[3-(fluoromethyl)oxetan-3- | | |
| | yl]methoxy]-2-methyl- | | |
| | pyrazolo[4,3-f]quinazolin-9-yl]- | | |
| | N,N-dimethyl-4,6,7,8- | | |
| | tetrahydropyrazolo[1,5- | | |
| | a][1,4]diazepine-2- | | |
| | carboxamide | | |
| Intermediate I-180 and | Example 207 | 3.77 min, | 9.89 (s, 1H), 8.57 |
| 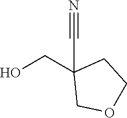 3-(hydroxymethyl) oxolane-3- carbonitrile | 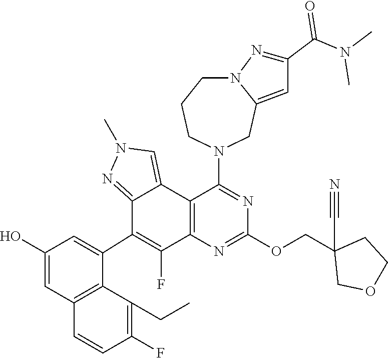 [SMILES: CCc1c(F)ccc2cc(O)cc(-c3c(F)c4nc(OCC5(C#N)CCOC5)nc(N5CCCn6nc(C(=O)N(C)C)cc6C5)c4c4cn(C)nc34)c12] | 722.7 [M + H]+ (Method 1) | (s, 1H), 7.78 (dd, J = 9.1, 6.1 Hz, 1H), 7.41-7.29 (m, 2H), 7.03 (d, J = 2.6 Hz, 1H), 6.44-6.36 (m, 1H), 5.05-4.79 (m, 2H), 4.57-4.42 (m, 3H), 4.42-4.31 (m, 1H), 4.19-4.00 (m, 5H), 4.00-3.83 (m, 4H), 3.27 (s, 3H), 2.94 (s, 3H), 2.48- 2.33 (m, 1H), 2.31- 2.02 (m, 5H), 0.59 (t, J = 7.4 Hz, 3H) |
| | Example 207 | | |
| | 5-[7-[(3-cyanotetrahydrofuran- | | |
| | 3-yl)methoxy]-4-(8-ethyl-7- | | |
| | fluoro-3-hydroxy-1-naphthyl)-5- | | |
| | fluoro-2-methyl-pyrazolo[4,3- | | |
| | f]quinazolin-9-yl]-N,N-dimethyl- | | |
| | 4,6,7,8-tetrahydropyrazolo[1,5- | | |
| | a][1,4]diazepine-2- | | |
| | carboxamide | | |
| Intermediate I-180 and | Example 208 | 3.72 min, | 9.87 (s, 1H), 8.55 |
| 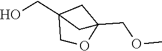 A-31 [1-(methoxymethyl)- 2-oxabicyclo[2.1.1] hexan-4-yl]methanol | 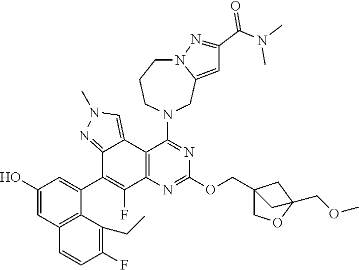 [SMILES: CCc1c(F)ccc2cc(O)cc(-c3c(F)c4nc(OCC56COC(COC)(C5)C6)nc(N5CCCn6nc(C(=O)N(C)C)cc6C5)c4c4cn(C)nc34)c12] | 753.7 [M + H]+ (Method 1) | (s, 1H), 7.78 (dd, J = 9.2, 6.1 Hz, 1H), 7.39-7.29 (m, 2H), 7.03 (d, J = 2.5 Hz, 1H), 6.29 (s, 1H), 5.01-4.77 (m, 2H), 4.63-4.44 (m, 4H), 4.14-3.99 (m, 4H), 3.98-3.86 (m, 1H), 3.63 (s, 2H), 3.53 (s, 2H), 3.28-3.24 (m, 6H), 2.93 (s, 3H), 2.46-2.36 (m, 1H), 2.25-2.08 (m, 3H), 1.76-1.70 (m, 2H), 1.51-1.46 (m, 2H), 0.59 (t, J = 7.3 |
| | Example 208 | | Hz, 3H) |
| | 5-[4-(8-ethyl-7-fluoro-3- | | |
| | hydroxy-1-naphthyl)-5-fluoro-7- | | |
| | [[1-(methoxymethyl)-2- | | |
| | oxabicyclo[2.1.1]hexan-4- | | |
| | yl]methoxy]-2-methyl- | | |
| | pyrazolo[4,3-f]quinazolin-9-yl]- | | |
| | N,N-dimethyl-4,6,7,8- | | |
| | tetrahydropyrazolo[1,5- | | |
| | a][1,4]diazepine-2- | | |
| | carboxamide | | |
| Intermediate I-182 | Example 209 | 3.13 min, | 9.77(s, 1H), 8.44(s, |
| 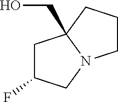 [SMILES: OC[C@@]12CCCN1C[C@H](F)C2] ((2R,7aS)-2- fluorohexahydro-1H- pyrrolizin-7a- yl)methanol | 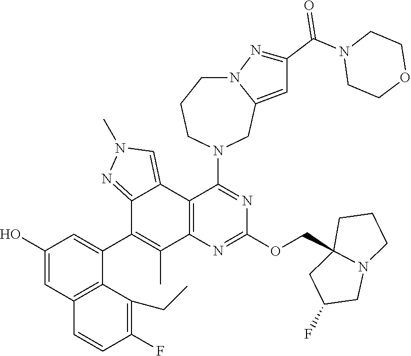 [SMILES: CCc1c(F)ccc2cc(O)cc(-c3c(C)c4nc(OC[C@@]56CCCN5C[C@H](F)C6)nc(N5CCCn6nc(C(=O)N7CCOCC7)cc6C5)c4c4cn(C)nc34)c12] | 792.6 [M + H]+ (Method 1) | 1H), 7.77-7.74(m, 1H), 7.33-7.28(m, 2H), 6.87-6.86(d, J = 2.60 Hz, 1H), 6.24(s, 1H), 5.33- 5.20 (d, J = 52.49 Hz, 1H), 4.94-4.76(dd, J = 56.81, 16.00 Hz, 2H), 4.52(s, 2H), 4.09-4.05(m, 4H), 4.00-3.97(m, 3H), 3.87-3.81(m, 1H), 3.58(s, 6H), 3.07- 2.99(m, 3H), 2.82(s, 1H), 2.29- 2.24(m, 2H), 2.19(s, 3H), 2.14- 1.99(m, 5H), 1.84- 1.75(m, 4H), 0.44- 0.41(t, J = 7.30 Hz, 3H). |
| | Example 209 | | |
| | [5-[4-(8-ethyl-7-fluoro-3- | | |
| | hydroxy-1-naphthyl)-7- | | |
| | [[(2R,8S)-2-fluoro-1,2,3,5,6,7- | | |
| | hexahydropyrrolizin-8- | | |
| | yl]methoxy]-2,5-dimethyl- | | |
| | pyrazolo[4,3-f]quinazolin-9-yl]- | | |
| | 4,6,7,8-tetrahydropyrazolo[1,5- | | |
| | a][1,4]diazepin-2-yl]- | | |
| | morpholino-methanone | | |
| Intermediate I-178 and | Example 211 | 3.09 min, | 9.79 (s, 1H), 8.53 |
| 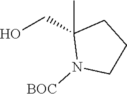 (2S)-1-BOC-2- Methylpyrrolidine-2- methanol | 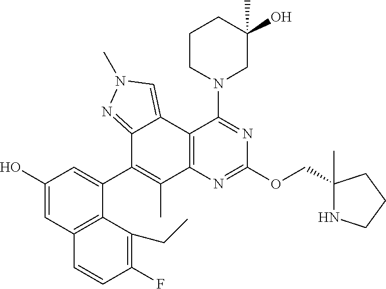 [SMILES: CCc1c(F)ccc2cc(O)cc(-c3c(C)c4nc(OC[C@]5(C)CCCN5)nc(N5CCC[C@@](C)(O)C5)c4c4cn(C)nc34)c12] | 613.3 [M + H]+ (Method 1) | (s, 1H), 7.75 (dd, J = 6.2, 9.1 Hz, 1H), 7.20-7.41 (m, 2H), 6.86 (s, 1H), 4.64 (s, 1H), 4.11-4.24 (m, 2H), 4.06 (s, 3H), 3.34- 3.66 (m, 7H), 2.76- 2.99 (m, 2H), 1.97- 2.39 (m, 7H), 1.55- 1.86 (m, 6H), 1.55- 1.42 (m, 1H), 0.93- 1.17 (m, 3H), 0.32- 0.46 (m, 3H). |
| | Example 211 | | |
| | (3R)-1-[4-(8-ethyl-7-fluoro-3- | | |
| | hydroxy-1-naphthyl)-2,5- | | |
| | dimethyl-7-[[rac-(2S)-2- | | |
| | methylpyrrolidin-2- | | |
| | yl]methoxy]pyrazolo[4,3- | | |
| | f]quinazolin-9-yl]-3-methyl- | | |
| | piperidin-3-ol | | |
| Intermediate I-184 and | Example 215 | 3.80 min, | 9.99-9.86 (m, 1H), |
| 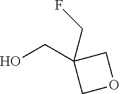 (3- (fluoromethyl)oxetan- 3-yl)methanol | 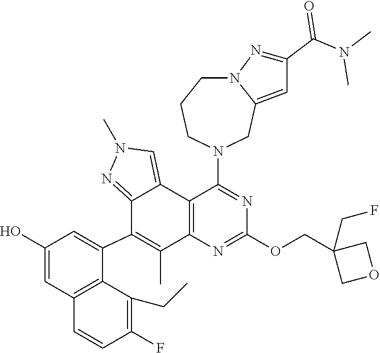 [SMILES: CCc1c(F)ccc2cc(O)cc(-c3c(C)c4nc(OCC5(CF)COC5)nc(N5CCCn6nc(C(=O)N(C)C)cc6C5)c4c4cn(C)nc34)c12] | 711.7 [M + H]+ (Method 1) | 8.57-8.51 (m, 1H), 7.82-7.73 (m, 1H), 7.38-7.28 (m, 2H), 7.02 (d, J = 2.6 Hz, 1H), 5.16- 5.07 (m, 1H), 4.75 (d, J = 47.2 Hz, 2H), 4.68-4.60 (m, 2H), 4.60-4.53 (m, 2H), 4.52-4.43 (m, 2H), 4.18-3.70 (m, 9H), 3.57-3.40 (m, 2H), 2.46-2.34 (m, 1H), 2.22-2.08 (m, 1H), 0.99 (s, 2H), 0.81 (s, 1H), 0.62- 0.51 (m, 3H). |
| | Example 215 | | |
| | 5-[4-(8-ethyl-7-fluoro-3- | | |
| | hydroxy-1-naphthyl)-7-[[3- | | |
| | (fluoromethyl)oxetan-3- | | |
| | yl]methoxy]-2,5-dimethyl- | | |
| | pyrazolo[4,3-f]quinazolin-9-yl]- | | |
| | N,N-dimethyl-4,6,7,8- | | |
| | tetrahydropyrazolo[1,5- | | |
| | a][1,4]diazepine-2- | | |
| | carboxamide | | |
| Intermediate I-186 and | Example 216 | 2.58 min, | 9.76 (1H, br s), |
| 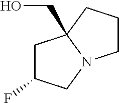 [SMILES: OC[C@@]12CCCN1C[C@H](F)C2] ((2R,7aS)-2- fluorohexahydro-1H- pyrrolizin-7a- yl)methanol | 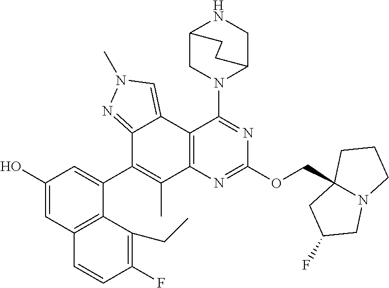 [SMILES: CCc1c(F)ccc2cc(O)cc(-c3c(C)c4nc(OC[C@@]56CCCN5C[C@H](F)C6)nc(N5CC6CCC5CN6)c4c4cn(C)nc34)c12] | 654.6 [M + H]+ (Method 1) | 8.29 (1H, d, J = 8.0 Hz), 7.74 (1H, dd, J = 9.0, 6.1 Hz), 7.33-7.26 (2H, m), 6.87-6.84 (1H, m), 5.37-5.19 (1H, m), 4.33-4.27 (1H, m), 4.18-4.11 (1H, m), 4.04 (3H, d, J = 1.7 Hz), 4.04-3.98 (1H, m), 3.80-3.72 (1H, m), 3.61-3.46 (1H, m), 3.29-3.21 (1H, m) 3.15-2.93 (5H, m), 2.87-2.78 (1H, m), 2.30-1.63 (16H, m), 0.41- 0.34 (3H, m). |
| | Example 216 | | |
| | 4-[9-(2,5- | | |
| | diazabicyclo[2.2.2]octan-2-yl)- | | |
| | 2,5-dimethyl-7-[[(2R,8S)-2- | | |
| | fluoro-1,2,3,5,6,7- | | |
| | hexahydropyrrolizin-8- | | |
| | yl]methoxy]pyrazolo[4,3- | | |
| | f]quinazolin-4-yl]-5-ethyl-6- | | |
| | fluoro-naphthalen-2-ol | | |
| Intermediate I-140 and | Example 221 | 3.90 min, | 9.88-9.83 (m, 1H), |
| 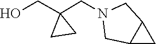 [SMILES: OCC1(CN2CC3CC3C2)CC1] [1-(3- azabicyclo[3.1.0] hexan-3- ylmethyl)cyclopropyl] methanol | 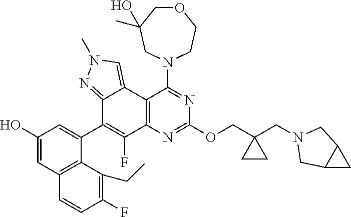 [SMILES: CCc1c(F)ccc2cc(O)cc(-c3c(F)c4nc(OCC5(CN6CC7CC7C6)CC5)nc(N5CCOCC(C)(O)C5)c4c4cn(C)nc34)c12] | 686.0 [M + H]⁺ (Method 1) | 8.53-8.49 (m, 1H), 7.81-7.75 (m, 1H), 7.37-7.29 (m, 2H), 7.03-6.98 (m, 1H), 5.35-5.23 (m, 1H), 4.32-4.19 (m, 2H), 4.08 (s, 3H), 4.00- 3.68 (m, 5H), 3.57- 3.39 (m, 2H), 2.98- 2.90 (m, 2H), 2.45- 2.34 (m, 3H), 2.27- 2.07 (m, 3H), 1.34- 1.22 (m, 4H), 1.01 (s, 1H), 0.87-0.79 |
| | Example 221 | | (m, 1H), 0.62-0.49 |
| | 4-[7-[[1-(3- | | (m, 6H), 0.41-0.33 |
| | azabicyclo[3.1.0]hexan-3- | | (m, 2H), 0.25-0.17 |
| | ylmethyl)cyclopropyl]methoxy]- | | (m, 1H). |
| | 4-(8-ethyl-7-fluoro-3-hydroxy- | | |
| | 1-naphthyl)-5-fluoro-2-methyl- | | |
| | pyrazolo[4,3-f]quinazolin-9-yl]- | | |
| | 6-methyl-1,4-oxazepan-6-ol | | |
| Intermediate I-140 and | Example 222 | 3.16 min, | 9.87-9.85 (d, J = |
| 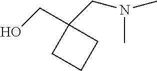 (1-[(dimethyl- amino)methyl] cyclobutyl)methanol | 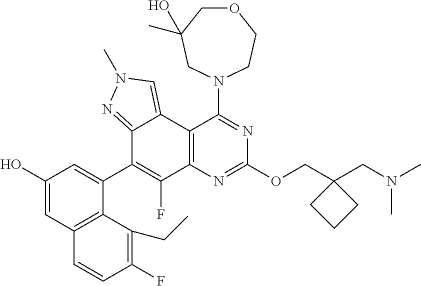 [SMILES: CCc1c(F)ccc2cc(O)cc(-c3c(F)c4nc(OCC5(CN(C)C)CCC5)nc(N5CCOCC(C)(O)C5)c4c4cn(C)nc34)c12] | 661.6 [M + H]⁺ (Method 1) | 5.60 Hz, 1H), 8.52- 8.51 (d, J = 3.60 Hz, 1H), 7.79-7.75 (m, 1H), 7.36-7.31 (m, 2H), 7.03-7.02 (d, J = 2.48 Hz, 1H), 5.41-5.38 (d, J = 11.26 Hz, 1H), 4.50-4.29 (m, 2H), 4.08 (s, 3H), 3.99- 3.70 (m, 6H), 3.56- 3.38 (m, 3H), 2.41-2.40 (m, 2H), 2.11 (s, 6H), 1.98-1.79 (m, 7H), 1.03 (s, 2H), 0.86 (s, 1H), 0.59-0.55 (t, J = 7.30 Hz, 3H). |
| | Example 222 | | |
| | 4-[7-[[1-[(dimethylamino) | | |
| | methyl]cyclobutyl] | | |
| | methoxy]-4-(8-ethyl-7- | | |
| | fluoro-3-hydroxy-1-naphthyl)-5- | | |
| | fluoro-2-methyl-pyrazolo[4,3- | | |
| | f]quinazolin-9-yl]-6-methyl-1,4- | | |
| | oxazepan-6-ol | | |
| Intermediate I-188 and | Example 223 | 2.96 min, | 9.86 (s, 1H), 8.53 |
| 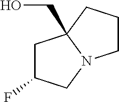 [SMILES: OC[C@@]12CCCN1C[C@H](F)C2] ((2R,7aS)-2- fluorohexahydro-1H- pyrrolizin-7a- yl)methanol | 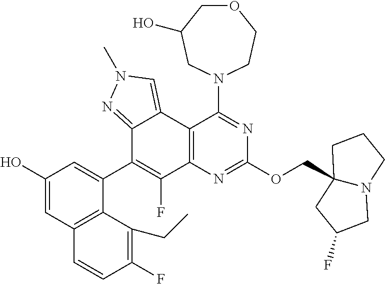 | 633.5 [M + H]⁺ (Method 1) | (d, J = 3.9 Hz, 1H), 7.77 (dd, J = 8.8, 6.0 Hz, 1H), 7.38- 7.26 (m, 2H), 7.01 (d, J = 2.6 Hz, 1H), 5.32 (d, J = 52.7 Hz, 1H), 5.18- 5.04 (m, 1H), 4.28- 3.99 (m, 8H), 3.95-3.50 (m, 8H), 3.25-2.79 (m, 4H), 2.44- 2.35 (m, 1H), 2.27- 1.73 (m, 7H), 0.60-0.52 (m, 3H). |
| | Example 223 | | |
| | 4-[4-(8-ethyl-7-fluoro-3- | | |
| | hydroxy-1-naphthyl)-5-fluoro-7- | | |
| | [[(2R,8S)-2-fluoro-1,2,3,5,6,7- | | |
| | hexahydropyrrolizin-8- | | |
| | yl]methoxy]-2-methyl- | | |
| | pyrazolo[4,3-f]quinazolin-9-yl]- | | |
| | 1,4-oxazepan-6-ol | | |
| Intermediate I-140 and | Example 224 | 2.99 min, | 9.86-9.85 (d, J = |
| 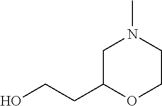 2-(4-methylmorpholin- 2-yl)ethanol | 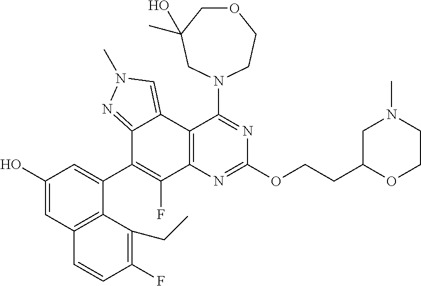 [SMILES: CCc1c(F)ccc2cc(O)cc(-c3c(F)c4nc(OCCC5CN(C)CCO5)nc(N5CCOCC(C)(O)C5)c4c4cn(C)nc34)c12] | 663.5 [M + H]⁺ (Method 1) | 5.56 Hz, 1H), 8.52- 8.51 (d, J = 4.08 Hz, 1H), 7.79-7.75 (m, 1H), 7.35-7.31 (m, 2H), 7.02-7.01 (d, J = 2.56 Hz, 1H), 5.24-5.20 (m, 1H), 4.48-4.37 (m, 2H), 4.08 (s, 3H), 3.99-3.70 (m, 6H), 3.57-3.40 (m, 4H), 2.74-2.69 (m, 1H), 2.58-2.55 (m, 1H), 2.42-2.35 (m, 2H), 2.15-2.13 (m, 4H), 1.97-1.83 (m, 3H), 1.75-1.70 (m, 1H), 1.00 (s, 2H), 0.82 |
| | Example 224 | | (s, 1H), 0.58-0.54 |
| | 4-[4-(8-ethyl-7-fluoro-3- | | (t, J = 7.32 Hz, 3H). |
| | hydroxy-1-naphthyl)-5-fluoro-2- | | |
| | methyl-7-[2-(4- | | |
| | methylmorpholin-2- | | |
| | yl)ethoxy]pyrazolo[4,3- | | |
| | f]quinazolin-9-yl]-6-methyl-1,4- | | |
| | oxazepan-6-ol | | |
| Intermediate I-177 and | Example 230 | 3.13 min, | 9.85 (d, J = 1.8 Hz, |
| 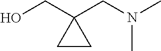 (1-((dimethyl- amino)methyl) cyclopropyl)methanol | 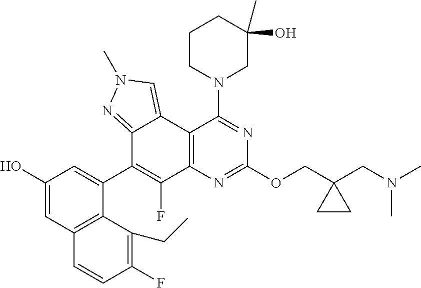 [SMILES: CCc1c(F)ccc2cc(O)cc(-c3c(F)c4nc(OCC5(CN(C)C)CC5)nc(N5CCC[C@@](C)(O)C5)c4c4cn(C)nc34)c12] | 631.8 [M + H]⁺ (Method 1) | 1H), 8.68-8.58 (m, 1H), 7.80-7.75 (m, 1H), 7.36-7.30 (m, 2H), 7.01 (d, J = 2.6 Hz, 1H), 4.62-4.59 (m, 1H), 4.30-4.25 (m, 2H), 4.08 (s, 3H), 3.66-3.19 (m, 6H), 2.44-2.13 (m, 9H), 1.80-1.59 (m, 3H), 1.11-1.00 (m, 3H), 0.67 (s, 2H), 0.59-0.53 (m, 3H), 0.43 (s, 2H). |
| | Example 230 | | |
| | (3R)-1-[7-[[1-[(dimethyl- | | |
| | amino)methyl]cyclopropyl] | | |
| | methoxy]-4-(8-ethyl-7- | | |
| | fluoro-3-hydroxy-1-naphthyl)-5- | | |
| | fluoro-2-methyl-pyrazolo[4,3- | | |
| | f]quinazolin-9-yl]-3-methyl- | | |
| | piperidin-3-ol | | |
| Intermediate I-140 and | Example 232 | 2.89 min, | 9.86-9.85 (d, J = |
| 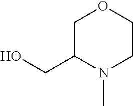 (4-methylmorpholin-3- yl)methanol | 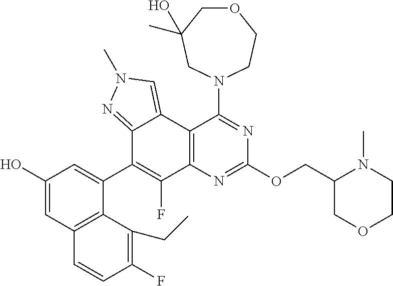 [SMILES: CCc1c(F)ccc2cc(O)cc(-c3c(F)c4nc(OCC5COCCN5C)nc(N5CCOCC(C)(O)C5)c4c4cn(C)nc34)c12] | 649.5 [M + H]⁺ (Method 1) | 5.28 Hz, 1H), 8.53- 8.52 (d, J = 4.04 Hz, 1H), 7.79-7.75 (m, 1H), 7.35-7.31 (m, 2H), 7.02-7.01 (d, J = 2.44 Hz, 1H), 5.24-5.19 (m, 1H), 4.60-4.46 (m, 1H), 4.39-4.27 (m, 1H), 4.08 (s, 3H), 3.96- 3.66 (m, 8H), 3.55- 3.40 (m, 4H), 2.31- 2.30 (d, J = 3.36 Hz, 3H), 2.26-2.12 (m, 3H), 1.24 (s, 2H), 1.00 (s, 2H), 0.87-0.82 (m, 1H), 0.59-0.55 (m, 3H). |
| | Example 232 | | |
| | 4-[4-(8-ethyl-7-fluoro-3- | | |
| | hydroxy-1-naphthyl)-5-fluoro-2- | | |
| | methyl-7-[(4-methylmorpholin- | | |
| | 3-yl)methoxy]pyrazolo[4,3- | | |
| | f]quinazolin-9-yl]-6-methyl-1,4- | | |
| | oxazepan-6-ol | | |
| Intermediate I-140 and | Example 233 | 2.88 min, | 9.86-9.85 (d, J = |
| 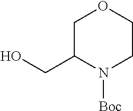 [SMILES: CC(C)(C)OC(=O)N1CCOCC1CO] 4-boc-(3- hydroxymethyl) morpholine | 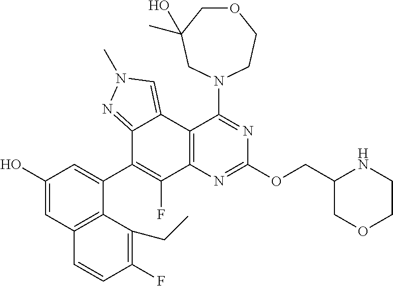 [SMILES: CCc1c(F)ccc2cc(O)cc(-c3c(F)c4nc(OCC5COCCN5)nc(N5CCOCC(C)(O)C5)c4c4cn(C)nc34)c12] | 635.5 [M + H]⁺ (Method 1) | 6.04 Hz, 1H), 8.53- 8.52 (d, J = 4.36 Hz, 1H), 7.79-7.75 (m, 1H), 7.35-7.31 (m, 2H), 7.02-7.01 (d, J = 2.64 Hz, 1H), 5.13-5.12 (d, J = 4.68 Hz, 1H), 4.28- 4.27 (m, 2H), 4.08 (s, 3H), 3.92-3.68 (m, 8H), 3.54-3.39 (m, 3H), 3.18 (s, 1H), 2.87-2.80 (m, 2H), 2.41-2.36 (m, 2H), 2.14-2.08 (m, 1H), 0.98 (s, 2H), 0.79 (s, 1H), 0.58- 0.55 (t, J = 7.34 Hz, |
| | Example 233 | | 3H). |
| | 4-[4-(8-ethyl-7-fluoro-3- | | |
| | hydroxy-1-naphthyl)-5-fluoro-2- | | |
| | methyl-7-(morpholin-3- | | |
| | ylmethoxy)pyrazolo[4,3- | | |
| | f]quinazolin-9-yl]-6-methyl-1,4- | | |
| | oxazepan-6-ol | | |
| Intermediate I-178 and | Example 234 | 3.15 and | 9.79 (s, 1H), 8.53 |
| 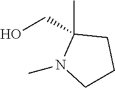 [(2S)-1,2- dimethylpyrrolidin-2- yl]methanol | 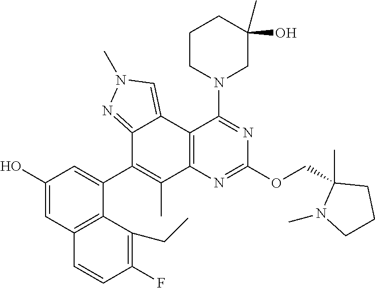 [SMILES: CCc1c(F)ccc2cc(O)cc(-c3c(C)c4nc(OC[C@]5(C)CCCN5C)nc(N5CCC[C@@](C)(O)C5)c4c4cn(C)nc34)c12] | 3.18 min, 627.5 [M + H]⁺ (Method 1) | (s, 1H), 7.75 (dd, J = 6.2, 8.9 Hz, 1H), 7.37-7.22 (m, 2H), 6.89-6.82 (m, 1H), 4.65 (s, 1H), 4.33 (m, 2H), 4.06 (s, 3H), 3.44- 3.39 (m, 3H), 2.93- 2.80 (m, 2H), 2.63- 2.54 (m, 3H), 2.28 (s, 3H), 2.24-2.19 (m, 3H), 2.01-1.89 (m, 2H), 1.75-1.52 (m, 6H), 1.40-1.19 (m, 3H), 1.07-1.01 (s, 3H), 0.45- 0.34 (m, 3H). |
| | Example 234 | | |
| Intermediate I-140 and | Example 235 | 3.17 min, | 9.86 (d, J = 6.4 Hz, |
| 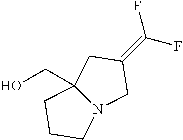 [SMILES: OCC12CCCN1CC(=C(F)F)C2] [6- (difluoromethylene)- 2,3,5,7-tetrahydro-1H- pyrrolizin-8- yl]methanol | 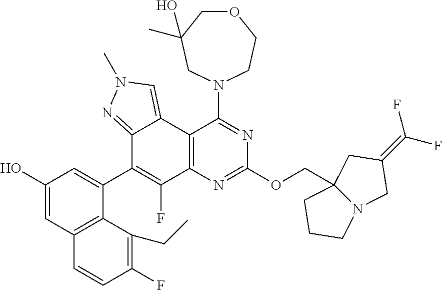 [SMILES: CCc1c(F)ccc2cc(O)cc(-c3c(F)c4nc(OCC56CCCN5CC(=C(F)F)C6)nc(N5CCOCC(C)(O)C5)c4c4cn(C)nc34)c12] | 707.5 [M + H]⁺ (Method 1) | 1H), 8.53 (d, J = 4.3 Hz, 1H), 7.80-7.75 (m, 1H), 7.37-7.31 (m, 2H), 7.03-7.00 (m, 1H), 5.32-5.23 (m, 1H), 4.30-4.03 (m, 3H), 4.08 (s, 3H), 4.00-3.37 (m, 10H), 3.05-2.95 (m, 1H), 2.64-2.35 (m, 2H), 2.21-1.73 (m, 6H), 1.03-0.82 (m, 3H), 0.60-0.53 (m, 3H). |
| | Example 235 | | |
| | 4-[7-[[6-(difluoromethylene)- | | |
| | 2,3,5,7-tetrahydro-1H- | | |
| | pyrrolizin-8-yl]methoxy]-4-(8- | | |
| | ethyl-7-fluoro-3-hydroxy-1- | | |
| | naphthyl)-5-fluoro-2-methyl- | | |
| | pyrazolo[4,3-f]quinazolin-9-yl]- | | |
| | 6-methyl-1,4-oxazepan-6-ol | | |
| Intermediate I-140 and | Example 236 | 3.13 and | 9.86 (d, J = 5.8 Hz, |
| 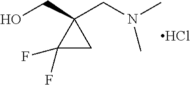 [(1S)-1- [(dimethylamino) methyl]-2,2-difluoro- cyclopropyl]methanol hydrochloride | 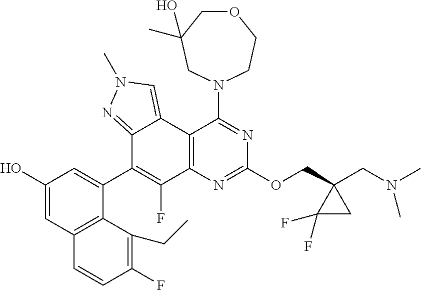 [SMILES: CCc1c(F)ccc2cc(O)cc(-c3c(F)c4nc(OC[C@@]5(CN(C)C)CC5(F)F)nc(N5CCOCC(C)(O)C5)c4c4cn(C)nc34)c12] | 3.16 min, 683.5 [M + H]⁺ (Method 1) | 1H), 8.53 (d, J = 4.2 Hz, 1H), 7.81-7.76 (m, 1H), 7.37-7.31 (m, 2H), 7.02 (d, J = 2.5 Hz, 1H), 5.23- 5.19 (m, 1H), 4.55- 4.43 (m, 2H), 4.09 (s, 3H), 4.01-3.69 (m, 6H), 3.56-3.40 (m, 2H), 2.68-2.59 (m, 1H), 2.45-2.29 (m, 2H), 2.18 (s, 7H), 1.85-1.76 (m, 1H), 1.52-1.42 (m, 1H), 1.02-0.83 (m, 3H), 0.57 (t, J = 7.3 Hz, 3H). |
| | Example 236 | | |
| | 4-[4-(8-ethyl-7-fluoro-3- | | |
| | hydroxy-1-naphthyl)-5-fluoro-2- | | |
| | methyl-7-[[(1S)-1- | | |
| | [(dimethylamino)methyl]-2,2- | | |
| | difluoro- | | |
| | cyclopropyl]methoxy]pyrazolo | | |
| | [4,3-f]quinazolin-9-yl]-6-methyl- | | |
| | 1,4-oxazepan-6-ol | | |
| Intermediate I-178 and | Example 239 | 2.97 and | 9.86 (d, J = 1.9 Hz, |
| 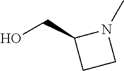 [(2S)-1- methylazetidin-2- yl]methanol | 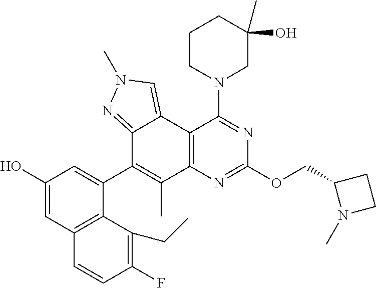 | 2.99 min, 603.5 [M + H]⁺ (Method 1) | 1H), 8.68-8.57 (m, 1H), 7.80-7.75 (m, 1H), 7.36-7.31 (m, 2H), 7.02 (d, J = 2.6 Hz, 1H), 4.65-4.52 (m, 1H), 4.41-4.31 (m, 2H), 4.08 (d, J = 0.8 Hz, 3H), 3.68- 3.57 (m, 1H), 3.52- 3.16 (m, 3H), 2.80- 2.72 (m, 1H), 2.47- 2.34 (m, 2H), 2.27 (d, J = 1.4 Hz, 3H), 2.22-1.58 (m, 8H), 1.09-0.98 (m, 3H), 0.60-0.53 (m, 3H). |
| | Example 239 | | |
| | (3R)-1-[4-(8-ethyl-7-fluoro-3- | | |
| | hydroxy-1-naphthyl)-5-fluoro-2- | | |
| | methyl-7-[[(2S)-1- | | |
| | methylazetidin-2- | | |
| | yl]methoxy]pyrazolo[4,3- | | |
| | f]quinazolin-9-yl]-3-methyl- | | |
| | piperidin-3-ol | | |
| Intermediate I-186 and | Example 240 | 3.06 and | 9.76 (1H, br s), |
| 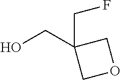 (3- (fluoromethyl)oxetan- 3-yl)methanol | 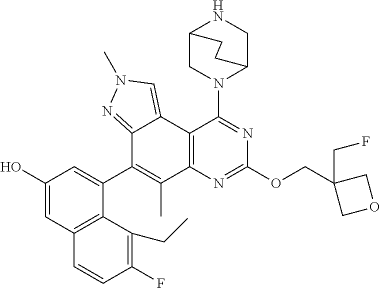 [SMILES: CCc1c(F)ccc2cc(O)cc(-c3c(C)c4nc(OCC5(CF)COC5)nc(N5CC6CCC5CN6)c4c4cn(C)nc34)c12] | 3.14 min, 615.6 [M + H]⁺ (Method 1) | 8.32 (1H, d, J = 7.0 Hz), 7.75 (1H, dd, J = 9.1, 6.1 Hz), 7.33-7.26 (2H, m), 6.86 (1H, dd, J = 4.0, 2.7 Hz), 4.75 (2H, d, J = 47.3 Hz, CH₂F), 4.67-4.60 (2H, m), 4.58-4.53 (2H, m), 4.52-4.47 (2H, m), 4.38-4.30 (1H, m), 4.05 (3H, d, J = 1.8 Hz), 3.82- 3.71 (1H, m), 3.64- 3.49 (1H, m), 3.39- 3.33 (1H, m), 3.28- 3.21 (1H, m), 3.08- 2.98 (2H, m), 2.32- |
| | Example 240 | | 2.15 (6H, m), 1.98- |
| | 4-[9-(2,5- | | 1.66 (3H, m) 0.42- |
| | diazabicyclo[2.2.2]octan-2-yl)- | | 0.34 (3H, m). |
| | 7-[[3-(fluoromethyl)oxetan-3- | | |
| | yl]methoxy]-2,5-dimethyl- | | |
| | pyrazolo[4,3-f]quinazolin-4-yl]- | | |
| | 5-ethyl-6-fluoro-naphthalen-2-ol | | |
| Intermediate I-190 and | Example 241 | 3.19 min, | 9.78 (s, 1H), 8.34 |
| 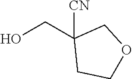 3-(hydroxymethyl) oxolane-3-carbonitrile | 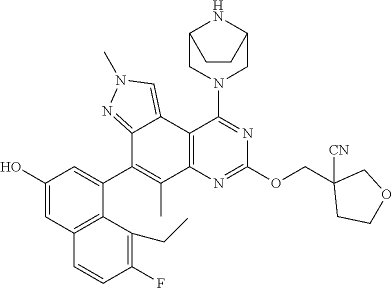 [SMILES: CCc1c(F)ccc2cc(O)cc(-c3c(C)c4nc(OCC5(C#N)CCOC5)nc(N5CC6CCC(C5)N6)c4c4cn(C)nc34)c12] | 622.5 [M + H]⁺ (Method 1) | (s, 1H), 7.75 (dd, J = 9.2 Hz, J = 6.0 Hz, 1H), 7.10 (t, J = 11.8 Hz, 1H), 6.85- 6.87 (m, 2H), 4.65 (q, J = 5.3 Hz, 1H), 4.54 (q, J = 5.1 Hz, 1H), 4.28 (br d, J = 4.2 Hz, 1H), 3.97- 4.15 (m, 8H), 3.84 (br d, J = 4.3 Hz, 1H), 3.65 (m, 3H), 2.48-2.55 (m, 1H), 2.25-2.35 (m, 6H), 2.05 (s, 1H), 1.81 (br s, |
| | Example 241 | | 4H), 1.26 (t, J = |
| | 3-[[9-(3,8- | | 7.14 Hz, 2H), |
| | diazabicyclo[3.2.1]octan-3-yl)- | | 0.48 (t, J = 7.36 |
| | 4-(8-ethyl-7-fluoro-3-hydroxy- | | Hz, 3H) |
| | 1-naphthyl)-2,5-dimethyl- | | |
| | pyrazolo[4,3-f]quinazolin-7- | | |
| | yl]oxymethyl]tetrahydrofuran-3- | | |
| | carbonitrile | | |
| Intermediate I-190 and | Example 242 | 3.00 min, | 9.76 (s, 1H), 8.29 |
| 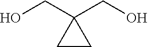 [1-(hydroxymethyl) cyclopropyl]methanol | 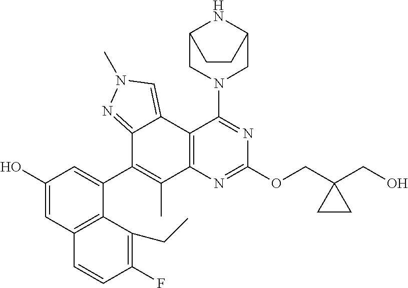 [SMILES: CCc1c(F)ccc2cc(O)cc(-c3c(C)c4nc(OCC5(CO)CC5)nc(N5CC6CCC(C5)N6)c4c4cn(C)nc34)c12] | 597.5 [M + H]⁺ (Method 1) | (s, 1H), 7.75 (dd, J = 9.1, 6.1 Hz, 1H), 7.34-7.24 (m, 2H), 6.86 (d, J = 2.6 Hz, 1H), 4.62 (t, J = 5.6 Hz, 1H), 4.29 (s, 2H), 4.16-4.06 (m, 1H), 4.03 (s, 3H), 3.79-3.70 (m, 1H), 3.62-3.32 (m, 6H), 2.32-2.12 (m, 5H), 1.90-1.52 (m, 4H), 0.59-0.47 (m, 4H), 0.38 (t, J = 7.3 Hz, 3H) |
| | Example 242 | | |
| | 4-[9-(3,8- | | |
| | diazabicyclo[3.2.1]octan-3-yl)- | | |
| | 7-[[1- | | |
| | (hydroxymethyl)cyclopropyl] | | |
| | methoxy]-2,5-dimethyl- | | |
| | pyrazolo[4,3-f]quinazolin-4-yl]- | | |
| | 5-ethyl-6-fluoro-naphthalen-2-ol | | |
| Intermediate I-192 and | Example 243 | 3.14 min, | 9.78 (s, 1H), 8.41 |
| 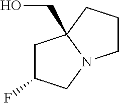 [SMILES: OC[C@@]12CCCN1C[C@H](F)C2] ((2R,7aS)-2- fluorohexahydro-1H- pyrrolizin-7a- yl)methanol | 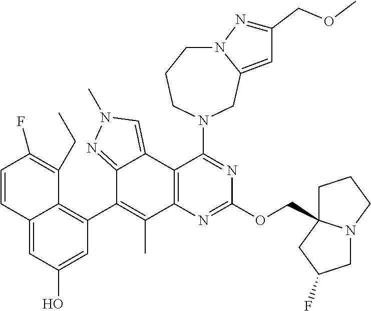 [SMILES: CCc1c(F)ccc2cc(O)cc(-c3c(C)c4nc(OC[C@@]56CCCN5C[C@H](F)C6)nc(N5CCCn6nc(COC)cc6C5)c4c4cn(C)nc34)c12] | 723.8 [M + H]⁺ (Method 1) | (s, 1H), 7.76 (dd, J = 9.1, 6.2 Hz, 1H), 7.34-7.26 (m, 2H), 6.87 (d, J = 2.4 Hz, 1H), 5.83 (s, 1H), 5.27 (d, J = 53.5 Hz, 1H), 4.88 (d, J = 16.1 Hz, 1H), 4.73 (d, J = 15.6 Hz, 1H), 4.51-4.38 (m, 2H), 4.21 (s, 2H), 4.13-4.06 (m, 1H), 4.04 (s, 3H), 4.03-3.96 (m, 1H), 3.87-3.77 (m, 1H), 3.19 (s, 3H), 3.15- 3.04 (m, 2H), 3.04- 2.96 (m, 1H), 2.87- 2.77 (m, 1H), 2.31- |
| | Example 243 | | 2.21 (m, 2H), 2.19 |
| | | | (s, 3H), 2.16-1.96 |
| | | | (m, 6H), 1.90-1.70 |
| | | | (m, 3H), 0.43 (t, J = |
| | | | 7.3 Hz, 3H) |
| Intermediate I-194 and | Example 244 | 3.48 min, | 9.88 (s, 1H), 8.54 |
| 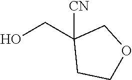 3-(hydroxymethyl) oxolane-3-carbonitrile | 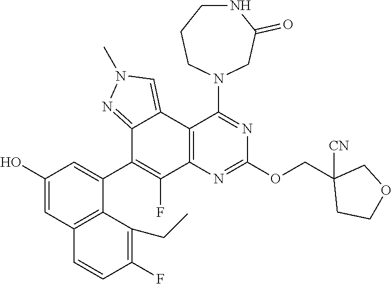 [SMILES: CCc1c(F)ccc2cc(O)cc(-c3c(F)c4nc(OCC5(C#N)CCOC5)nc(N5CCCNC(=O)C5)c4c4cn(C)nc34)c12] | 628.6 [M + H]⁺ (Method 1) | (s, 1H), 7.78 (dd, J = 9.2, 6.1 Hz, 1H), 7.67-7.59 (m, 1H), 7.38-7.30 (m 2H), 7.02 (d, J = 2.6 Hz, 1H), 4.72- 4.49 (m, 2H), 4.24 (s, 2H), 4.11-4.04 (m, 4H), 3.97-3.81 (m, 5H), 3.06-2.96 (m, 2H), 2.48-2.36 (m, 2H), 2.26-2.10 (m, 2H), 2.05-1.88 (m, 2H), 0.57 (t, J = 7.3 Hz, 3H) |
| | Example 244 | | |
| | 3-[[4-(8-ethyl-7-fluoro-3- | | |
| | hydroxy-1-naphthyl)-5-fluoro-2- | | |
| | methyl-9-(3-oxo-1,4-diazepan- | | |
| | 1-yl)pyrazolo[4,3-f]quinazolin- | | |
| | 7-yl]oxymethyl]tetrahydrofuran- | | |
| | 3-carbonitrile | | |
| Intermediate I-195 and | Example 245 | 3.04 min, | 9.86-9.85(d, |
| 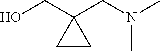 (1-((dimethyl- amino)methyl) cyclopropyl)methanol | 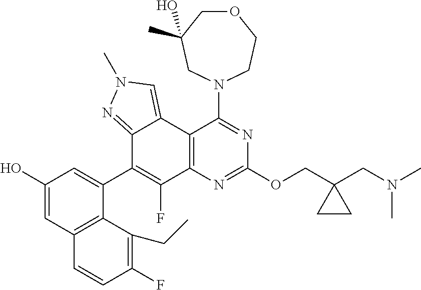 [SMILES: CCc1c(F)ccc2cc(O)cc(-c3c(F)c4nc(OCC5(CN(C)C)CC5)nc(N5CCOC[C@@](C)(O)C5)c4c4cn(C)nc34)c12] | 647.7 [M + H]⁺ (Method 1) | J = 6.08 Hz, 1H), 8.51(d, J = 3.48 Hz, 1H), 7.79-7.75(m, 1H), 7.35-7.31(m, 2H), 7.01(d, J = 2.68 Hz, 1H), 5.30(s, 1H), 4.31- 4.23(m, 2H), 4.07(s, 3H), 3.97- 3.70(m, 6H), 3.56- 3.40(m, 3H), 2.42- 2.35(m, 1H), 2.30- 2.21(m, 2H), 2.16(s, 6H), 1.02(s, 2H), 0.84(s, 1H), 0.64(s, 2H), 0.58- 0.54(t, J = 7.36 Hz, 3H), 0.42(s, 2H). |
| | Example 245 | | |
| | (6S)-4-[7-[[1-[(dimethyl- | | |
| | amino)methyl]cyclopropyl] | | |
| | methoxy]-4-(8-ethyl-7- | | |
| | fluoro-3-hydroxy-1-naphthyl)-5- | | |
| | fluoro-2-methyl-pyrazolo[4,3- | | |
| | f]quinazolin-9-yl]-6-methyl-1,4- | | |
| | oxazepan-6-ol | | |
| Intermediate I-190 and | Example 247 | 3.13 min, | 9.77 (s, 1H), 8.32 |
| 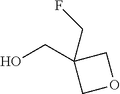 (3-(fluoromethyl)oxetan- 3-yl)methanol | 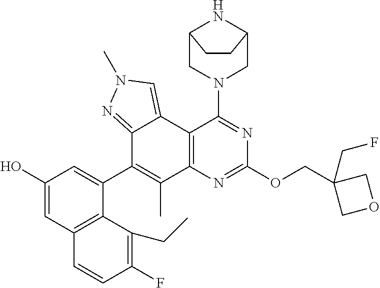 [SMILES: CCc1c(F)ccc2cc(O)cc(-c3c(C)c4nc(OCC5(CF)COC5)nc(N5CC6CCC(C5)N6)c4c4cn(C)nc34)c12] | 615.5 [M + H]⁺ (Method 1) | (s, 1H), 7.75 (dd, J = 9.1 Hz, J = 6.1 Hz, 1H), 7.31 (d, J = 9.1 Hz, 1H), 7.27 (d, J = 2.5 Hz, 1H), 6.87 (d, J = 2.6 Hz, 1H), 4.81 (s, 1H), 4.71 (s, 1H) 4.68- 4.61 (m, 2H), 4.57 (dd, J = 6.3 Hz, J = 2.8 Hz, 2H), 4.50 (d, J = 6.3 Hz, 2H), 4.19-4.10 (m, 1H), 4.04 (s, 3H), 3.79-3.70 (m, 1H), 3.62-3.55 (m, 1H), 3.42-3.47 (m, 3H), 2.31- |
| | Example 247 | | 2.09 (m, 5H), 1.80- |
| | 4-[9-(3,8- | | 1.84 (m, 1H), 1.57 |
| | diazabicyclo[3.2.1]octan-3-yl)- | | (br s, 3H), 0.38 (t, |
| | 7-[[3-(fluoromethyl)oxetan-3- | | J = 7.3 Hz, 3H). |
| | yl]methoxy]-2,5-dimethyl- | | |
| | pyrazolo[4,3-f]quinazolin-4-yl]- | | |
| | 5-ethyl-6-fluoro-naphthalen-2-ol | | |

### Example 184, (3R)-1-[4-(8-ethynyl-7-fluoro-3-hydroxy-1-naphthyl)-2,5-dimethyl-7-[[(2R,8S)-2-fluoro-1,2,3,5,6,7-hexahydropyrrolizin-8-yl]methoxy]pyrazolo[4,3-f]quinazolin-9-yl]-3-methyl-piperidin-3-ol

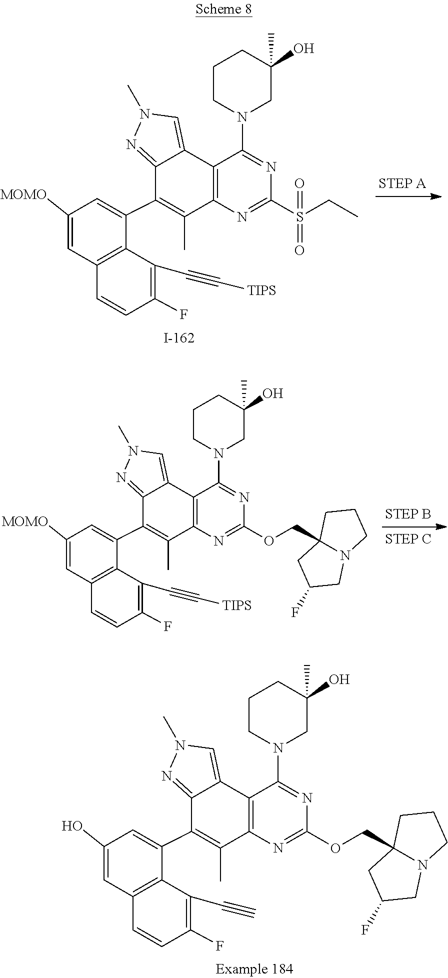

Step A, (3R)-1-[4-[7-fluoro-3-(methoxymethoxy)-8-(2-triisopropylsilylethynyl)-1-naphthyl]-2,5-dimethyl-7-[[(2R,8S)-2-fluoro-1,2,3,5,6,7-hexahydropyrrolizin-8-yl]methoxy]pyrazolo[4,3-f]quinazolin-9-yl]-3-methyl-piperidin-3-ol. A stirring solution of ((2R,7aS)-2-Fluorohexahydro-1H-pyrrolizin-7a-yl) methanol (143.02 mg, 0.9 mmol) in THF (4 mL) was cooled to 0° C. and Lithium bis(trimethylsilyl)amide in THF (1.12 mL, 1.12 mmol) added. The mixture was stirred for 15 mins and a solution of (3R)-1-[7-ethylsulfonyl-4-[7-fluoro-3-(methoxymethoxy)-8-(2-triisopropylsilylethynyl)-1-naphthyl]-2,5-dimethyl-pyrazolo[4,3-f]quinazolin-9-yl]-3-methyl-piperidin-3-ol (354. mg, 0.45 mmol) in THF (2 mL) was added dropwise. The ice bath was removed and the reaction mixture left to stir at room temperature for 2 hours. Water was added and the aqueous phase was extracted with DCM. The aqueous phase was extracted with further DCM (×2), followed by EtOAc (×3). The organic phases were washed with brine, passed through phase separator filter paper and reduced in vacuo. The crude material was purified by flash column chromatography (eluting in 0-100% EtOAc in pet. ether, followed by 0-20% MeOH in DCM) to afford the desired product (3R)-1-[4-[7-fluoro-3-(methoxymethoxy)-8-(2-triisopropylsilylethynyl)-1-naphthyl]-2,5-dimethyl-7-[(2R,8S)-2-fluoro-1,2,3,5,6,7-hexahydropyrrolizin-8-yl]methoxy]pyrazolo[4,3-f]quinazolin-9-yl]-3-methyl-piperidin-3-ol (192 mg, 0.2251 mmol, 50% yield) as a pale brown solid.

UPLC-MS (ES⁺, Method 2): 1.50 min.

Step B, (3R)-1-[4-[8-ethynyl-7-fluoro-3-(methoxymethoxy)-1-naphthyl]-2,5-dimethyl-7-[[(2R,8S)-2-fluoro-1,2,3,5,6,7-hexahydropyrrolizin-8-yl]methoxy]pyrazolo[4,3-f]quinazolin-9-yl]-3-methyl-piperidin-3-ol. Cesium Fluoride (170.93 mg, 1.13 mmol) was added to a solution of (3R)-1-[4-[7-fluoro-3-(methoxymethoxy)-8-(2-triisopropylsilylethynyl)-1-naphthyl]-2,5-dimethyl-7-[(2R,8S)-2-fluoro-1,2,3,5,6,7-hexahydropyrrolizin-8-yl]methoxy]pyrazolo[4,3-f]quinazolin-9-yl]-3-methyl-piperidin-3-ol (192. mg, 0.23 mmol) in DMF (2 mL) and the reaction left to stir for 3 hours. The reaction was quenched with a saturated solution of ammonium hydrochloride, EtOAc and water were added and the layers separated. The aqueous layer was extracted with further EtOAc. Organic fractions were collected, washed with brine, passed through phase separating filter paper and the solvent reduced in vacuo to afford (3R)-1-[4-[8-ethynyl-7-fluoro-3-(methoxymethoxy)-1-naphthyl]-2,5-dimethyl-7-[[(2R,8S)-2-fluoro-1,2,3,5,6,7-hexahydropyrrolizin-8-yl]methoxy]pyrazolo[4,3-f]quinazolin-9-yl]-3-methyl-piperidin-3-ol (156 mg, 0.2239 mmol, 99% yield) as a brown oil.

UPLC-MS (ES⁺, Method 5): 1.66 min, 697.3 [M+H]⁺.

Step C, (3R)-1-[4-(8-ethynyl-7-fluoro-3-hydroxy-1-naphthyl)-2,5-dimethyl-7-[[(2R,8S)-2-fluoro-1,2,3,5,6,7-hexahydropyrrolizin-8-yl]methoxy]pyrazolo[4,3-f]quinazolin-9-yl]-3-methyl-piperidin-3-ol. Hydrogen Chloride (0.56 mL, 2.24 mmol) was added to a stirring solution of (3R)-1-[4-[8-ethynyl-7-fluoro-3-(methoxymethoxy)-1-naphthyl]-2,5-dimethyl-7-[[(2R,8S)-2-fluoro-1,2,3,5,6,7-hexahydropyrrolizin-8-yl]methoxy]pyrazolo[4,3-f]quinazolin-9-yl]-3-methyl-piperidin-3-ol (156. mg, 0.22 mmol) in methanol (2 ml). The reaction mixture was warmed to 40° C. and left to stir overnight. The reaction mixture was reduced in vacuo and the crude material was purified by reverse phase chromatography (eluting in 0-50% acetonitrile (0.1% formic acid) in water (0.1% formic acid)). product containing fractions were loaded onto a pre-equilibrated SCX-2 column (2 g, washing with MeOH and eluting with 1M NH₃/MeOH) to afford the product (3R)-1-[4-(8-ethynyl-7-fluoro-3-hydroxy-1-naphthyl)-2,5-dimethyl-7-[[(2R,8S)-2-fluoro-1,2,3,5,6,7-hexahydropyrrolizin-8-yl]methoxy]pyrazolo[4,3-f]quinazolin-9-yl]-3-methyl-piperidin-3-ol (36 mg, 0.0552 mmol, 25% yield) as a pale orange solid.

UPLC-MS (ES⁺, Method 5): 2.87 min, 653.6 [M+H]⁺.

¹H NMR (400 MHz, DMSO-d6) δ/ppm: 10.04-9.97 (m, 1H), 8.49 (br s, 1H), 8.02-7.89 (m, 1H), 7.47-7.30 (m, 2H), 7.10-6.96 (m, 1H), 5.29 (d, J=54.8 Hz, 1H), 4.77-4.38 (m, 1H), 4.23-4.11 (m, 1H), 4.12-3.94 (m, 4H), 3.60-3.34 (m, 3H), 3.31-2.96 (m, 5H), 2.90-2.77 (m, 1H), 2.28-1.96 (m, 7H), 1.93-1.52 (m, 6H), 1.19-0.98 (m, 3H).

### Example 170, 1-[[1-[4-(8-ethyl-7-fluoro-3-hydroxy-1-naphthyl)-5-fluoro-9-(6-hydroxy-6-methyl-1,4-oxazepan-4-yl)-2-methyl-pyrazolo[4,3-f]quinazolin-7-yl]oxymethyl]cyclopropyl]methyl]pyrrolidine-3-carbonitrile

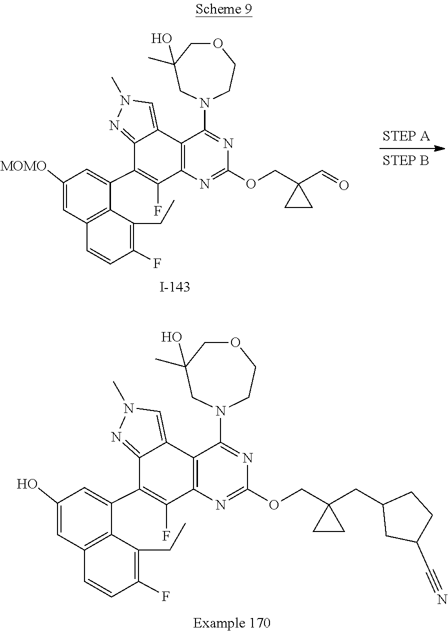

Step A, 1-[[1-[4-[8-ethyl-7-fluoro-3-(methoxymethoxy)-1-naphthyl]-5-fluoro-9-(6-hydroxy-6-methyl-1,4-oxazepan-4-yl)-2-methyl-pyrazolo[4,3-f]quinazolin-7-yl]oxymethyl]cyclopropyl]methyl]pyrrolidine-3-carbonitrile. To a solution of 1-[4-[8-ethyl-7-fluoro-3-(methoxymethoxy)-1-naphthyl]-5-fluoro-9-(6-hydroxy-6-methyl-1,4-oxazepan-4-yl)-2-methyl-pyrazolo[4,3-f]quinazolin-7-yl]oxymethyl]cyclopropanecarbaldehyde (30. mg, 0.05 mmol), Pyrrolidine-3-carbonitrile (6.54 mg, 0.07 mmol) and Acetic Acid (Glacial) (0.01 mL, 0.09 mmol) in DCM (3 mL) was added Sodium triacetoxyborohydride (28.83 mg, 0.14 mmol). The reaction mixture was left to stir at room temperature for 1 hour. The reaction mixture was quenched with NaHCO₃aq, extracted with DCM (×3) and washed with brine, dried over magnesium sulfate, filtered and concentrated in vacuo to afford 1-[1-[4-[8-ethyl-7-fluoro-3-(methoxymethoxy)-1-naphthyl]-5-fluoro-9-(6-hydroxy-6-methyl-1,4-oxazepan-4-yl)-2-methyl-pyrazolo[4,3-f]quinazolin-7-yl]oxymethyl]cyclopropyl]methyl]pyrrolidine-3-carbonitrile (37 mg, 0.0439 mmol, 97% yield) as a yellow oil.

UPLC-MS (ES⁺, Method 5): 1.74 min, 742.4 [M+H]⁺.

Step B, 1-[[1-[[4-(8-ethyl-7-fluoro-3-hydroxy-1-naphthyl)-5-fluoro-9-(6-hydroxy-6-methyl-1,4-oxazepan-4-yl)-2-methyl-pyrazolo[4,3-f]quinazolin-7-yl]oxymethyl]cyclopropyl]methyl]pyrrolidine-3-carbonitrile. A mixture of 1-[1-[4-[8-ethyl-7-fluoro-3-(methoxymethoxy)-1-naphthyl]-5-fluoro-9-(6-hydroxy-6-methyl-1,4-oxazepan-4-yl)-2-methyl-pyrazolo[4,3-f]quinazolin-7-yl]oxymethyl]cyclopropyl]methyl]pyrrolidine-3-carbonitrile (37. mg, 0.05 mmol), Triethylsilane (0.04 mL, 0.25 mmol) and Trifluoroacetic acid (0.11 mL, 1.5 mmol) in DCM (2 mL) was stirred at room temperature for 1 hour. The reaction mixture was concentrated under reduced pressure and purified by reverse phase chromatography (4 g, 0-50% MeCN in water (0.1% formic acid)). Product containing fractions were loaded onto a pre-equilibrated SCX-2 column (1 g, washing with MeOH and eluting in 1N NH₃/MeOH), concentrated and dried to afford the desired product 1-[1-[4-(8-ethyl-7-fluoro-3-hydroxy-1-naphthyl)-5-fluoro-9-(6-hydroxy-6-methyl-1,4-oxazepan-4-yl)-2-methyl-pyrazolo[4,3-f]quinazolin-7-yl]oxymethyl]cyclopropyl]methyl]pyrrolidine-3-carbonitrile (8.4 mg, 0.0114 mmol, 23% yield) as a yellow solid.

UPLC-MS (ES⁺, Method 1): 3.11 min, 698.6 [M+H]⁺.

¹H NMR (400 MHz, DMSO-d6) δ/ppm: 9.85-9.84 (d, J=6.08 Hz, 1H), 8.51 (d, J=3.48 Hz, 1H), 7.79-7.75 (m, 1H), 7.35-7.30 (m, 2H), 7.01-7.00 (m, 1H), 5.32-5.29 (m, 1H), 4.37-4.20 (m, 2H), 4.07 (s, 3H), 3.98-3.70 (m, 5H), 3.56-3.40 (m, 2H), 3.20 (s, 1H), 2.82-2.78 (m, 1H), 2.63-2.61 (m, 1H), 2.55-2.53 (m, 1H), 2.44-2.36 (m, 4H), 2.19-2.09 (m, 2H), 1.93-1.85 (m, 1H), 1.23 (s, 1H), 1.01 (s, 2H), 0.84 (s, 1H), 0.65 (s, 2H), 0.58-0.54 (t, J=7.28 Hz, 3H), 0.44 (s, 2H).

The following examples in table 24 were made by analogy with example 170 (scheme 9) starting from the intermediate and building blocks for step A as described in the table. Some of the examples in table 24 were synthesised with alternative reagents such as borane-2-methylpyridine complex.

TABLE 24

| Step A, | | UPLC-MS | ¹H NMR (400 |
|---|---|---|---|
| intermediate + | | (ES⁺): min, | MHz, DMSO-d₆) |
| building block | Example Structure/Name | m/z | δ/ppm |
| Intermediate I-143 and | Example 171 | 3.05 min, | 9.86-9.84 (d, |
| 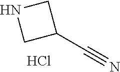 Azetidine-3- carbonitrile hydrochloride | 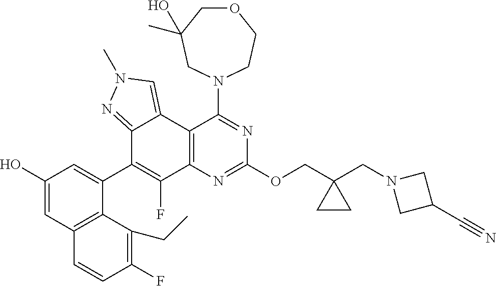 [SMILES: CCc1c(F)ccc2cc(O)cc(-c3c(F)c4nc(OCC5(CN6CC(C#N)C6)CC5)nc(N5CCOCC(C)(O)C5)c4c4cn(C)nc34)c12] | 684.5 [M + H]+ (Method 1) | J = 6.00 Hz, 1H), 8.52-8.51 (d, J = 3.68 Hz, 1H), 7.79-7.75 (m, 1H), 7.35-7.31 (m, 2H), 7.01 (m, 1H), 5.30-5.27 (m, 1H), 4.25- 4.17 (m, 2H), 4.07 (s, 3H), 3.95-3.70 (m, 6H), 3.56-3.41 (m, 5H), 3.26- 3.25 (m, 2H), 2.44-2.39 (m, 3H), 2.16 (s, 1H), 1.01 (s, 2H), 0.83 (s, 1H), 0.58-0.54 |
| | Example 171 | | (m, J = 7.80 Hz, |
| | 1-[[1-[[4-(8-ethyl-7-fluoro-3- | | 5H), 0.44 (s, 2H) |
| | hydroxy-1-naphthyl)-5-fluoro-9- | | |
| | (6-hydroxy-6-methyl-1,4- | | |
| | oxazepan-4-yl)-2-methyl- | | |
| | pyrazolo[4,3-f]quinazolin-7- | | |
| | yl]oxymethyl]cyclopropyl]methyl] | | |
| | azetidine-3-carbonitrile | | |
| Intermediate I-143 and | Example 172 | 3.19 min, | 9.86-9.84 (d, |
| 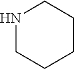 Piperidine | 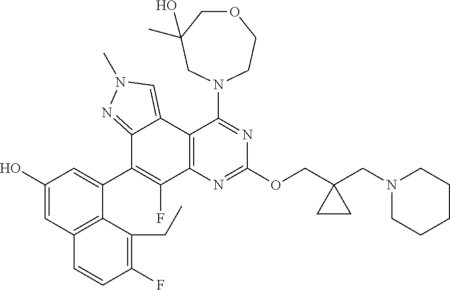 [SMILES: CCc1c(F)ccc2cc(O)cc(-c3c(F)c4nc(OCC5(CN6CCCCC6)CC5)nc(N5CCOCC(C)(O)C5)c4c4cn(C)nc34)c12] | 687.6 [M + H]+ (Method 1) | J = 6.36 Hz, 1H), 8.52-8.51 (d, J = 3.36 Hz, 1H), 7.79-7.75 (m, 1H), 7.35-7.31 (m, 2H), 7.01- 7.00 (m, 1H), 5.29-5.28 (m, 1H), 4.36-4.26 (m, 2H), 4.08 (s, 3H), 3.98-3.70 (m, 6H), 3.55- 3.37 (m, 3H), 2.44-2.34 (m, 3H), 2.34-2.26 (m, 3H), 2.16 (s, 1H), 1.42 (s, 4H), 1.33 (s, 2H), 1.01 |
| | Example 172 | | (s, 2H), 0.84 (s, |
| | 4-[4-(8-ethyl-7-fluoro-3-hydroxy- | | 1H), 0.63 (s, 2H), |
| | 1-naphthyl)-5-fluoro-2-methyl-7- | | 0.58-0.55 (t, |
| | [[1-(1- | | J = 7.36 Hz, 3H), |
| | piperidylmethyl)cyclopropyl] | | 0.38 (s, 2H) |
| | methoxy]pyrazolo[4,3-f]quinazolin-9- | | |
| | yl]-6-methyl-1,4-oxazepan-6-ol | | |
| Intermediate I-143 and | Example 189 | 3.24 min, | 9.86-9.84(d, |
| 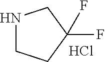 3,3-difluoropyrrolidine, HCl | 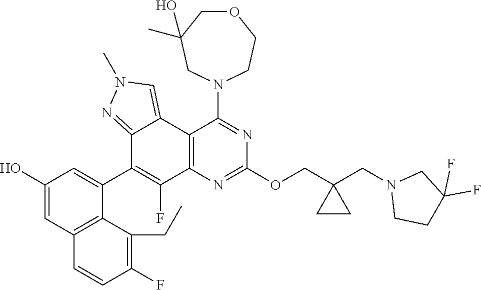 [SMILES: CCc1c(F)ccc2cc(O)cc(-c3c(F)c4nc(OCC5(CN6CCC(F)(F)C6)CC5)nc(N5CCOCC(C)(O)C5)c4c4cn(C)nc34)c12] | 709.5 [M + H]+ (Method 1) | J = 5.80 Hz, 1H), 8.52-8.51(d, J = 3.44 Hz, 1H), 7.79-7.75(m, 1H), 7.35-7.31(m, 2H), 7.01-7.00(d, J = 2.56 Hz, 1H), 5.28(s, 1H), 4.32- 4.25(m, 2H), 4.07(s, 3H), 3.97- 3.70(m, 6H), 3.55-3.40(m, 3H), 2.93-2.88(m, 2H), 2.72-2.69(t, J = 7.20 Hz, 2H), 2.44-2.39(m, 2H), 2.24-2.13(m, 3H), 1.01(s, 2H), |
| | Example 189 | | 0.83(s, 1H), 0.65- |
| | 4-[7-[[1-[(3,3-difluoropyrrolidin-1- | | 0.63(m, 2H), |
| | yl)methyl]cyclopropyl]methoxy]- | | 0.58-0.54(t, |
| | 4-(8-ethyl-7-fluoro-3-hydroxy-1- | | J = 7.41 Hz, 3H), |
| | naphthyl)-5-fluoro-2-methyl- | | 0.44(s, 2H), |
| | pyrazolo[4,3-f]quinazolin-9-yl]-6- | | |
| | methyl-1,4-oxazepan-6-ol | | |
| Intermediate I-143 and | Example 190 | 3.05 min, | 9.86-9.85(d, |
| 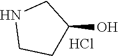 (S)-3- hydroxypyrrolidine hydrochloride | 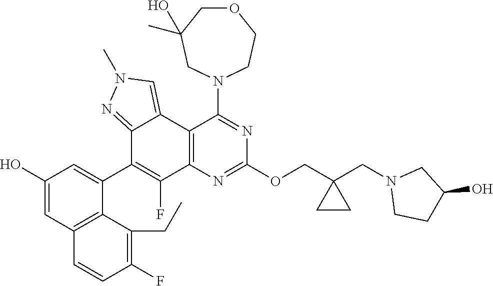 [SMILES: CCc1c(F)ccc2cc(O)cc(-c3c(F)c4nc(OCC5(CN6CC[C@H](O)C6)CC5)nc(N5CCOCC(C)(O)C5)c4c4cn(C)nc34)c12] | 689.6 [M + H]+ (Method 1) | J = 5.64 Hz, 1H), 8.51(d, J = 3.32 Hz, 1H), 7.79-7.75(m, 1H), 7.35-7.31(m, 2H), 7.01(d, J = 2.56 Hz, 1H), 5.32-5.29(m, 1H), 4.63-4.62(d, J = 4.16 Hz, 1H), 4.34-4.21(m, 2H), 4.15(s, 1H), 4.07(s, 3H), 3.97- 3.70(m, 6H), 3.56-3.35(m, 3H), 2.76-2.70(m, 1H), 2.42-2.37(m, 4H),2.30-2.29(m, |
| | Example 190 | | 1H), 2.19-2.11(m, |
| | 4-[4-(8-ethyl-7-fluoro-3-hydroxy- | | 1H), 1.97-1.88(m, |
| | 1-naphthyl)-5-fluoro-2-methyl-7- | | 1H), 1.52-1.45(m, |
| | [[1-[[(3S)-3-hydroxypyrrolidin-1- | | 1H), 1.01(s, 2H), |
| | yl]methyl]cyclopropyl]methoxy] | | 0.84(s, 1H), |
| | pyrazolo[4,3-f]quinazolin-9-yl]-6- | | 0.61(s, 2H), 0.58- |
| | methyl-1,4-oxazepan-6-ol | | 0.54(t, H = 7.32 |
| | | | Hz, 3H), 0.41 |
| | | | (s, 2H), |
| Intermediate I-143 and | Example 191 | 3.18 min, | 9.88-9.83 (m, |
| 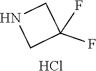 3,3-Difluoroazetidine hydrochloride | 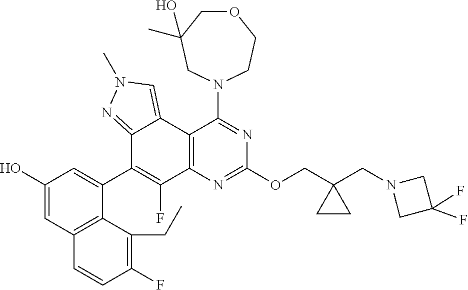 [SMILES: CCc1c(F)ccc2cc(O)cc(-c3c(F)c4nc(OCC5(CN6CC(F)(F)C6)CC5)nc(N5CCOCC(C)(O)C5)c4c4cn(C)nc34)c12] | 695.6 [M + H]+ (Method 1) | 1H), 8.52 (d, J = 5.0 Hz, 1H), 7.78 (dd, J = 6.0, 9.5 Hz, 1H), 7.36- 7.31 (m, 2H), 7.02 (d, J = 3.1 Hz, 1H), 5.28 (s, 1H), 4.29-4.20 (m, 2H), 4.11-4.06 (m, 3H), 4.00- 3.70 (m, 6H), 3.60 (t, J = 12.4 Hz, 4H), 3.54- 3.41 (m, 2H), 2.64-2.55 (m, 2H), 2.45-2.35 (m, 1H), 2.23- 2.10 (m, 1H), |
| | Example 191 | | 1.01 (s, 2H), 0.87- |
| | 4-[7-[[1-[(3,3-difluoroazetidin-1- | | 0.84 (m, 1H), |
| | yl)methyl]cyclopropyl]methoxy]- | | 0.61-0.54 (m, |
| | 4-(8-ethyl-7-fluoro-3-hydroxy-1- | | 5H), 0.51-0.45 |
| | naphthyl)-5-fluoro-2-methyl- | | (m, 2H). |
| | pyrazolo[4,3-f]quinazolin-9-yl]-6- | | |
| | methyl-1,4-oxazepan-6-ol | | |
| Intermediate I-143 and | Example 192 | 1.93 min, | 9.86-9.85(d, |
| 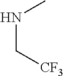 N-Methyl-2,2,2- trifluoroethylamine | 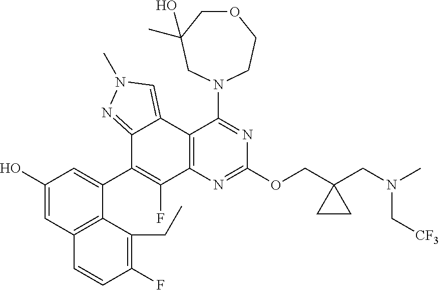 | 689.6 [M + H]+ (Method 1) | J = 5.96 Hz, 1H), 8.52-8.51(d, J = 3.60 Hz, 1H), 7.79-7.75(m, 1H), 7.35-7.31(m, 2H), 7.02-7.01(m, 1H), 5.35-5.27(d, H = 29.24 Hz, 1H), 4.37-4.22(m, 2H), 4.07(s, 3H), 3.96- 3.73(m, 6H), 3.54-3.38(m, 3H), 3.24-3.16(m, 2H), 2.61-2.60(m, 2H), 2.41(s, 3H), 2.19- 2.09(m, 1H), 1.00(s, 2H), 0.81(m, 1H), |
| | Example 192 | | 0.65(m, 2H), |
| | 4-[4-(8-ethyl-7-fluoro-3-hydroxy- | | 0.58-0.54(t, |
| | 1-naphthyl)-5-fluoro-2-methyl-7- | | J = 7.30 Hz, 3H), |
| | [[1-[[methyl(2,2,2- | | 0.44(s, 2H), |
| | trifluoroethyl)amino]methyl] | | |
| | cyclopropyl]methoxy]pyrazolo[4,3- | | |
| | f]quinazolin-9-yl]-6-methyl-1,4- | | |
| | oxazepan-6-ol | | |
| Intermediate I-143 and | Example 193 | 3.13 min, | 9.87-9.84 (m, |
| 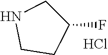 (3R)-3- Fluoropyrrolidine hydrochloride (1:1) | 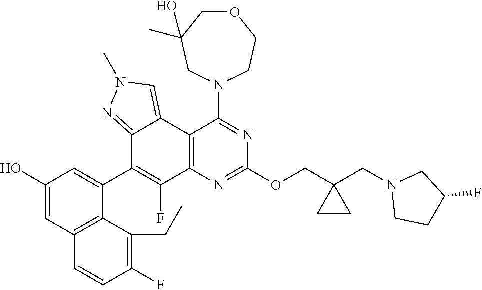 [SMILES: CCc1c(F)ccc2cc(O)cc(-c3c(F)c4nc(OCC5(CN6CC[C@@H](F)C6)CC5)nc(N5CCOCC(C)(O)C5)c4c4cn(C)nc34)c12] | 691.6 [M + H]+ (Method 1) | 1H), 8.52 (d, J = 4.0 Hz, 1H), 7.80-7.76 (m, 1H), 7.36-7.30 (m, 2H), 7.01 (d, J = 3.2 Hz, 1H), 5.31-5.28 (m, 1H), 5.15 (td, J = 6.1, 55.7 Hz, 1H), 4.37-4.21 (m, 2H), 4.12- 4.06 (m, 3H), 3.98-3.69 (m, 5H), 3.56-3.40 (m, 2H), 2.91- 2.74 (m, 2H), 2.47-2.29 (m, 4H), 2.21-1.97 |
| | Example 193 | | (m, 2H), 1.90- |
| | 4-[4-(8-ethyl-7-fluoro-3-hydroxy- | | 1.74 (m, 1H), |
| | 1-naphthyl)-5-fluoro-2-methyl-7- | | 1.02 (s, 2H), 0.85 |
| | [[1-[[(3R)-3-fluoropyrrolidin-1- | | (s, 1H), 0.67- |
| | yl]methyl]cyclopropyl]methoxy] | | 0.61 (m, 2H), |
| | pyrazolo[4,3-f]quinazolin-9-yl]-6- | | 0.57 (t, J = 7.1 Hz, |
| | methyl-1,4-oxazepan-6-ol | | 3H), 0.46-0.40 |
| | | | (m, 2H) |
| Intermediate I-143 and | Example 194 | 3.03 min, | 9.88-9.84 (m, |
| 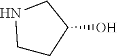 (R)-3-Pyrrolidinol | 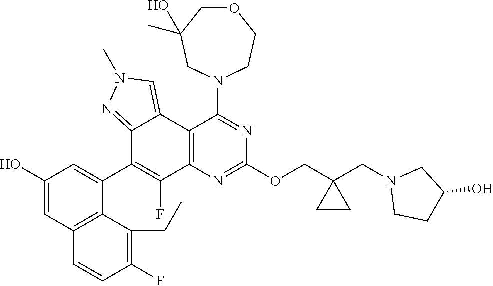 [SMILES: CCc1c(F)ccc2cc(O)cc(-c3c(F)c4nc(OCC5(CN6CC[C@@H](O)C6)CC5)nc(N5CCOCC(C)(O)C5)c4c4cn(C)nc34)c12] | 689.6 [M + H]+ (Method 1) | 1H), 8.56-8.48 (m, 1H), 7.80- 7.74 (m, 1H), 7.38-7.29 (m, 2H), 7.01 (d, J = 2.5 Hz, 1H), 5.35- 5.26 (m, 1H), 4.64 (br s, 1H), 4.36-4.20 (m, 2H), 4.19-4.11 (m, 1H), 4.08 (s, 3H), 3.99-3.68 (m, 6H), 3.57- 3.36 (m, 3H), 2.79-2.68 (m, 1H), 2.60-2.53 (m, 1H), 2.45- 2.27 (m, 4H), |
| | Example 194 | | 2.21-2.09 (m, |
| | 4-[4-(8-ethyl-7-fluoro-3-hydroxy- | | 1H), 2.03-1.87 |
| | 1-naphthyl)-5-fluoro-2-methyl-7- | | (m, 1H), 1.55- |
| | [[1-[[(3R)-3-hydroxypyrrolidin-1- | | 1.42 (m, 1H), |
| | yl]methyl]cyclopropyl]methoxy] | | 1.06-0.80 (m, |
| | pyrazolo[4,3-f]quinazolin-9-yl]-6- | | 3H), 0.62 (s, 2H), |
| | methyl-1,4-oxazepan-6-ol | | 0.56 (t, J = 7.3 |
| | | | Hz, 3H), 0.42 (s, |
| | | | 2H). |
| Intermediate I-143 and | Example 212 | 3.10 min, | 9.86-9.85 (m, |
| 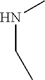 N-Ethylmethylamine | 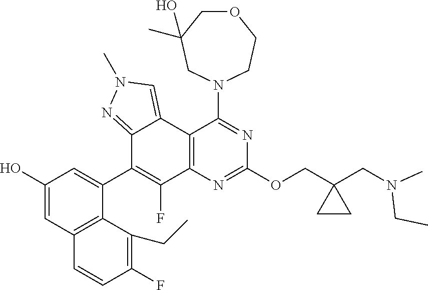 [SMILES: CCc1c(F)ccc2cc(O)cc(-c3c(F)c4nc(OCC5(CN(C)CC)CC5)nc(N5CCOCC(C)(O)C5)c4c4cn(C)nc34)c12] | 661.6 [M + H]⁺ (Method 1) | 1H), 8.51-8.50 (m, 1H), 7.79- 7.75 (m, 1H), 7.35-7.31 (m, 2H), 7.02-7.01 (m, 1H), 5.33- 5.30 (d, J = 14.88 Hz, 1H), 4.33-4.22 (m, 2H), 4.07(s, 3H), 3.96-3.70 (m, 6H), 3.55-3.37 (m, 2H), 2.38- 2.34 (m, 3H), 2.26-2.23 (m, 1H), 2.16 (s, 3H), 1.24 (m, 1H), 1.01 (s, 2H), |
| | Example 212 | | 0.93-0.83 (m, |
| | 4-[4-(8-ethyl-7-fluoro-3-hydroxy- | | 5H), 0.63 (s, 2H), |
| | 1-naphthyl)-7-[[1- | | 0.58-0.54 (t, |
| | [[ethyl(methyl)amino]methyl] | | J = 7.36 Hz, 3H), |
| | cyclopropyl]methoxy]-5-fluoro-2- | | 0.44 (s, 2H). |
| | methyl-pyrazolo[4,3-f]quinazolin- | | |
| | 9-yl]-6-methyl-1,4-oxazepan-6-ol | | |
| Intermediate I-143 and | Example 213 | 3.03 min, | 9.86-9.84 (d, J = |
| 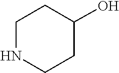 piperidin-4-ol | 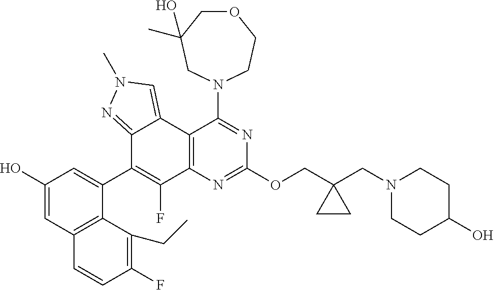 [SMILES: CCc1c(F)ccc2cc(O)cc(-c3c(F)c4nc(OCC5(CN6CCC(O)CC6)CC5)nc(N5CCOCC(C)(O)C5)c4c4cn(C)nc34)c12] | 702.8 [M + H]⁺ (Method 1) | 5.60 Hz, 1H), 8.52-8.51 (d, J = 3.28 Hz, 1H), 7.79-7.75 (m, 1H), 7.35-7.31 (m, 2H), 7.02- 7.01 (m, 1H), 5.30-5.27 (d, J = 12.76 Hz, 1H), 1.47 (s, 1H), 4.37-4.25 (m, 2H), 4.07 (s, 3H), 3.98-3.70 (m, 6H), 3.55-3.36 (m, 3H), 2.75 (s, 2H), 2.30-2.22 (m, 2H), 2.16- 2.10 (m, 2H), |
| | Example 213 | | 1.98 (s, 2H), |
| | 4-[4-(8-ethyl-7-fluoro-3-hydroxy- | | 1.66-1.63 (m, |
| | 1-naphthyl)-5-fluoro-7-[[1-[(4- | | 2H), 1.32 (s, 2H), |
| | hydroxy-1- | | 1.01 (s, 2H), 0.83 |
| | piperidyl)methyl]cyclopropyl] | | (m, 1H), 0.63 (m, |
| | methoxy]-2-methyl-pyrazolo[4,3- | | 2H), 0.59-0.55 (t, |
| | f]quinazolin-9-yl]-6-methyl-1,4- | | J = 7.30 Hz, 3H), |
| | oxazepan-6-ol | | 0.38 (s, 2H). |
| Intermediate I-143 and | Example 214 | 3.08 min, | 9.86 (d, J = 6.1 |
| 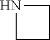 azetidine | 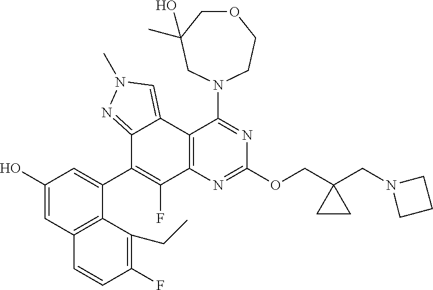 [SMILES: CCc1c(F)ccc2cc(O)cc(-c3c(F)c4nc(OCC5(CN6CCC6)CC5)nc(N5CCOCC(C)(O)C5)c4c4cn(C)nc34)c12] | 659.6 [M + H]⁺ (Method 1) | Hz, 1H), 8.52 (d, J = 3.6 Hz, 1H), 7.80-7.75 (m, 1H), 7.36-7.31 (m, 2H), 7.02 (d, J = 3.2 Hz, 1H), 5.28 (s, 1H), 4.27- 4.19 (m, 2H), 4.13-4.04 (m, 3H), 4.01-3.70 (m, 6H), 3.56- 3.40 (m, 2H), 3.19-3.05 (m, 4H), 2.43-2.35 (m, 4H), 2.22- 2.09 (m, 1H), 2.01-1.89 (m, 2H), 1.02-0.84 |
| | Example 214 | | (m, 3H), 0.59- |
| | 4-[7-[[1-(azetidin-1- | | 0.53 (m, 5H), |
| | ylmethyl)cyclopropyl]methoxy]- | | 0.46-0.39 (m, |
| | 4-(8-ethyl-7-fluoro-3-hydroxy-1- | | 2H). |
| | naphthyl)-5-fluoro-2-methyl- | | |
| | pyrazolo[4,3-f]quinazolin-9-yl]-6- | | |
| | methyl-1,4-oxazepan-6-ol | | |
| Intermediate I-143 and | Example 217 | 3.10 min, | 9.88-9.84 (1H, |
| 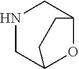 8-Oxa-3- azabicyclo[3,2,1]octane | 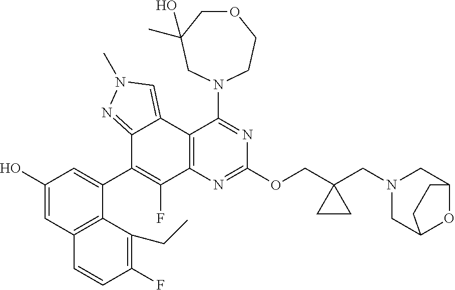 [SMILES: CCc1c(F)ccc2cc(O)cc(-c3c(F)c4nc(OCC5(CN6CC7CCC(C6)O7)CC5)nc(N5CCOCC(C)(O)C5)c4c4cn(C)nc34)c12] | 715.5 [M + H]⁺ (Method 1) | m), 8.53-8.50 (1H, m), 7.81- 7.75 (1H, m), 7.37-7.31 (2H, m), 7.03-6.99 (1H, m), 5.39- 5.31 (1H, m), 4.47-4.27 (2H, m), 4.15-4.07 (5H, m), 3.98- 3.69 (6H, m), 3.56-3.41 (2H, m), 2.65-2.57 (2H, m), 2.41- 2.35 (1H, m), 2.31-2.21 (2H, m), 2.20-2.09 (3H, m), 1.75- |
| | Example 217 | | 1.51 (4H, m), |
| | 4-[4-(8-ethyl-7-fluoro-3-hydroxy- | | 1.01 (2H, s), |
| | 1-naphthyl)-5-fluoro-2-methyl-7- | | 0.87-0.82 (1H, |
| | [[1-(8-oxa-3- | | m), 0.66-0.60 |
| | azabicyclo[3.2.1]octan-3- | | (2H, m), 0.60- |
| | ylmethyl)cyclopropyl]methoxy] | | 0.53 (3H, m), |
| | pyrazolo[4,3-f]quinazolin-9-yl]-6- | | 0.40-0.33 (2H, |
| | methyl-1,4-oxazepan-6-ol | | m). |
| Intermediate I-143 and | Example 218 | 3.32 min, | 9.86-9.84 (m, |
| 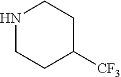 4- trifluoromethyl- pipeirdine | 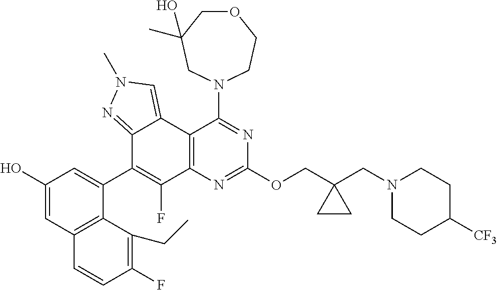 [SMILES: CCc1c(F)ccc2cc(O)cc(-c3c(F)c4nc(OCC5(CN6CCC(C(F)(F)F)CC6)CC5)nc(N5CCOCC(C)(O)C5)c4c4cn(C)nc34)c12] | 755.5 [M + H]⁺ (Method 1) | 1H), 8.52-8.50 (m, 1H), 7.80- 7.75 (m, 1H), 7.37-7.30 (m, 2H), 7.02-6.99 (m, 1H), 5.33- 5.26 (m, 1H), 4.37-4.26 (m, 2H), 4.08 (s, 3H), 4.01-3.69 (m, 6H), 3.56-3.37 (m, 4H), 3.08- 2.97 (m, 2H), 2.26-2.08 (m, 2H), 1.91-1.82 (m, 2H), 1.75- 1.66 (m, 2H), 1.43-1.29 (m, |
| | Example 218 | | 2H), 1.01 (br s, |
| | 4-[4-(8-ethyl-7-fluoro-3-hydroxy- | | 2H), 0.83 (br s, |
| | 1-naphthyl)-5-fluoro-2-methyl-7- | | 1H), 0.66 (br s, |
| | [[1-[[4-(trifluoromethyl)-1- | | 2H), 0.57 (t, J = |
| | piperidyl]methyl]cyclopropyl] | | 7.7 Hz, 3H), 0.41 |
| | methoxy]pyrazolo[4,3-f]quinazolin-9- | | (br s, 2H) |
| | yl]-6-methyl-1,4-oxazepan-6-ol | | |
| Intermediate I-143 and | Example 219 | 3.64 min, | 9.87-9.84 (m, |
| 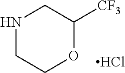 2-trifluoro- methylmorpholine hydrochloride | 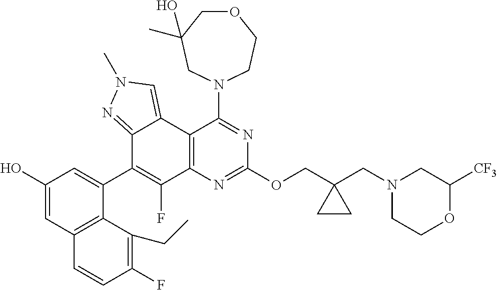 [SMILES: CCc1c(F)ccc2cc(O)cc(-c3c(F)c4nc(OCC5(CN6CCOC(C(F)(F)F)C6)CC5)nc(N5CCOCC(C)(O)C5)c4c4cn(C)nc34)c12] | 757.5 [M + H]⁺ (Method 1) | 1H), 8.53-8.51 (m, 1H), 7.80- 7.75 (m, 1H), 7.37-7.30 (m, 2H), 7.02-6.99 (m, 1H), 5.32- 5.26 (m, 1H), 4.41-4.34 (m, 1H), 4.32-4.26 (m, 1H), 4.11- 4.04 (m, 3H), 4.00-3.69 (m 7H), 3.56-3.38 (m, 5H), 3.04- 2.96 (m, 1H), 2.86-2.77 (m, 1H), 2.21-1.97 (m, 3H), 1.01 (br |
| | Example 219 | | s, 2H), 0.86-0.81 |
| | 4-[4-(8-ethyl-7-fluoro-3-hydroxy- | | (m, 1H), 0.72- |
| | 1-naphthyl)-5-fluoro-2-methyl-7- | | 0.64 (m, 2H), |
| | [[1-[[2- | | 0.57 (t, J = 7.4 |
| | (trifluoromethyl)morpholin-4- | | Hz, 3H), 0.44 (br |
| | yl]methyl]cyclopropyl]methoxy] | | s, 2H) Several H |
| | pyrazolo[4,3-f]quinazolin-9-yl]-6- | | overlap with |
| | methyl-1,4-oxazepan-6-ol | | DMSO peak. |
| Intermediate I-143 and | Example 225 | 3.15 min, | 9.87-9.84 (m, 1H), |
| 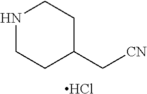 2-(piperidin-4- yl)acetonitrile hydrochloride | 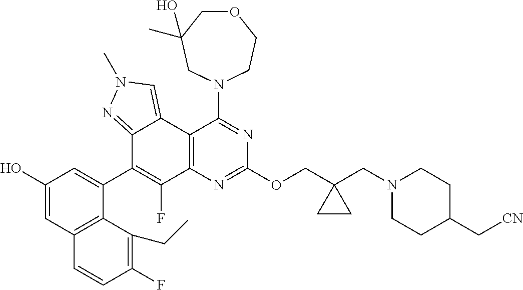 [SMILES: CCc1c(F)ccc2cc(O)cc(-c3c(F)c4nc(OCC5(CN6CCC(CC#N)CC6)CC5)nc(N5CCOCC(C)(O)C5)c4c4cn(C)nc34)c12] | 726.6 [M + H]⁺ (Method 1) | 8.52-8.50 (m, 1H), 7.80-7.75 (m, 1H), 7.37-7.30 (m, 2H), 7.03-7.00 (m, 1H), 5.29 (s, 1H), 4.33- 4.25 (m, 2H), 4.08 (s, 3H), 3.98-3.69 (m, 6H), 3.57-3.39 (m, 3H), 2.99-2.90 (m, 2H), 1.89-1.80 (m, 2H), 1.67-1.59 (m, 2H), 1.59-1.50 (m, 1H), 1.26-1.16 (m, 2H), 1.01 (s, 2H), 0.84 (s, 1H), 0.65 (s, 2H), 0.57 (t, J = 7.5 Hz, 3H), 0.42-0.37 (m, 2H). |
| | Example 225 | | Several H overlap |
| | 2-[1-[[1-[[4-(8-ethyl-7-fluoro-3- | | with DMSO peak. |
| | hydroxy-1-naphthyl)-5-fluoro-9- | | |
| | (6-hydroxy-6-methyl-1,4- | | |
| | oxazepan-4-yl)-2-methyl- | | |
| | pyrazolo[4,3-f]quinazolin-7- | | |
| | yl]oxymethyl]cyclopropyl]methyl]- | | |
| | 4-piperidyl]acetonitrile | | |
| Intermediate I-143 and | Example 226 | 3.06 min, | 9.87-9.84 (m, 1H), |
| 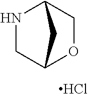 (1S,4S)-2-oxa-5- azabicyclo[2.2.1] heptane hydrochloride | 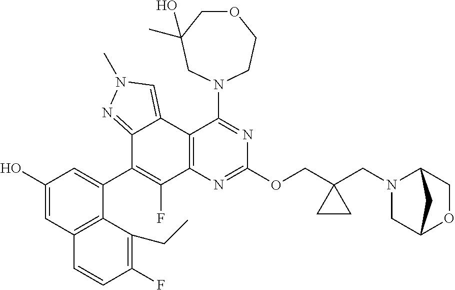 [SMILES: CCc1c(F)ccc2cc(O)cc(-c3c(F)c4nc(OCC5(CN6C[C@@H]7C[C@H]6CO7)CC5)nc(N5CCOCC(C)(O)C5)c4c4cn(C)nc34)c12] | 701.6 [M + H]⁺ (Method 1) | 8.52-8.50 (m, 1H), 7.80-7.75 (m, 1H), 7.37-7.30 (m, 2H), 7.03-7.00 (m, 1H), 5.31-5.26 (m, 1H), 4.40-4.23 (m, 3H), 4.08 (s, 3H), 3.99- 3.69 (m, 6H), 3.57- 3.37 (m, 4H), 2.87- 2.81 (m, 1H), 2.23- 2.09 (m, 2H), 1.68- 1.62 (m, 1H), 1.54- 1.48 (m, 1H), 1.01 (s, 2H), 0.84 (s, 1H), 0.63-0.53 (m, 5H), 0.50-0.36 (m, 2H). Several H overlap with |
| | Example 226 | | DMSO peak. |
| | 4-[4-(8-ethyl-7-fluoro-3-hydroxy- | | |
| | 1-naphthyl)-5-fluoro-2-methyl-7- | | |
| | [[1-[[rac-(1S,4S)-2-oxa-5- | | |
| | azabicyclo[2.2.1]heptan-5- | | |
| | yl]methyl]cyclopropyl]methoxy] | | |
| | pyrazolo[4,3-f]quinazolin-9-yl]-6- | | |
| | methyl-1,4-oxazepan-6-ol | | |
| Intermediate I-143 and | Example 227 | 3.18 min, | 9.87-9.84 (m, 1H), |
| 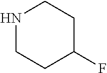 4-fluoropiperidine | 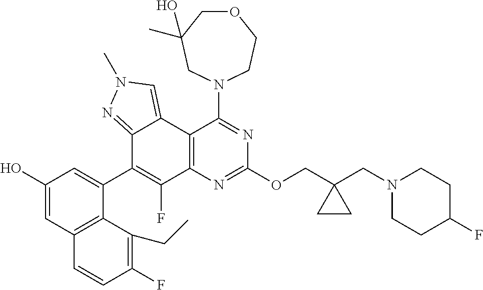 [SMILES: CCc1c(F)ccc2cc(O)cc(-c3c(F)c4nc(OCC5(CN6CCC(F)CC6)CC5)nc(N5CCOCC(C)(O)C5)c4c4cn(C)nc34)c12] | 705.6 [M + H]⁺ (Method 1) | 8.53-8.51 (m, 1H), 7.80-7.75 (m, 1H), 7.36-7.31 (m, 2H), 7.02-7.00 (m, 1H), 5.31-5.26 (m, 1H), 4.70-4.52 (m, 1H), 4.38-4.27 (m, 2H), 4.08 (s, 3H), 4.01- 3.69 (m, 6H), 3.57- 3.38 (m, 2H), 2.36- 2.25 (m, 3H), 2.21- 2.10 (m, 1H), 1.86- 1.72 (m, 2H), 1.67- 1.57 (m, 2H), 1.01 (s, 2H), 0.84 (s, 1H), 0.67-0.63 (m, 2H), 0.57 (t, J = 7.6 Hz, 3H), 0.42-0.37 |
| | Example 227 | | (m, 2H) Several H |
| | 4-[4-(8-ethyl-7-fluoro-3-hydroxy- | | under DMSO peak. |
| | 1-naphthyl)-5-fluoro-7-[[1-[(4- | | |
| | fluoro-1- | | |
| | piperidyl)methyl]cyclopropyl] | | |
| | methoxy]-2-methyl-pyrazolo[4,3- | | |
| | f]quinazolin-9-yl]-6-methyl-1,4- | | |
| | oxazepan-6-ol | | |
| Intermediate I-143 and | Example 228 | 3.16 min, | 9.87-9.84 (m, 1H), |
| 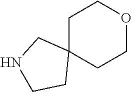 8-oxa-2- azaspiro[4.5]decane | 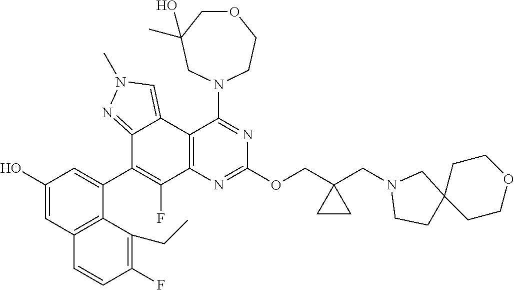 [SMILES: CCc1c(F)ccc2cc(O)cc(-c3c(F)c4nc(OCC5(CN6CCC7(CCOCC7)C6)CC5)nc(N5CCOCC(C)(O)C5)c4c4cn(C)nc34)c12] | 743.6 [M + H]⁺ (Method 1) | 8.52-8.50 (m, 1H), 7.80-7.75 (m, 1H), 7.37-7.30 (m, 2H), 7.02-6.99 (m, 1H), 5.31-5.25 (m, 1H), 4.36-4.28 (m, 2H), 4.08 (s, 3H), 4.00- 3.68 (m, 6H), 3.56- 3.36 (m, 6H), 2.23- 2.09 (m, 2H), 1.56- 1.49 (m, 2H), 1.47- 1.32 (m, 4H), 1.01 (s, 2H), 0.83 (s, 1H), 0.64-0.60 (m, 2H), 0.57 (t, J = 7.6 Hz, 3H), 0.44-0.37 (m, 2H). DMSO peal overlaps with |
| | Example 228 | | 6H. |
| | 4-[4-(8-ethyl-7-fluoro-3-hydroxy- | | |
| | 1-naphthyl)-5-fluoro-2-methyl-7- | | |
| | [[1-(8-oxa-2-azaspiro[4.5]decan- | | |
| | 2- | | |
| | ylmethyl)cyclopropyl]methoxy] | | |
| | pyrazolo[4,3-f]quinazolin-9-yl]-6- | | |
| | methyl-1,4-oxazepan-6-ol | | |
| Intermediate I-143 and | Example 229 | 3.23 min, | 9.86 (d, J = 6.5 Hz, |
| 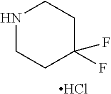 4,4-difluoropiperidine hydrochloride | 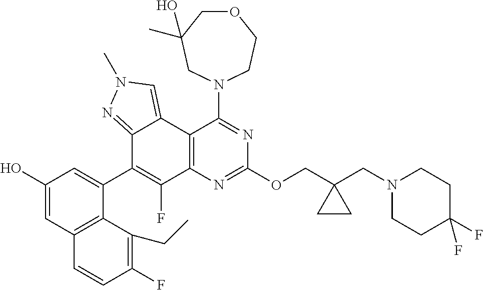 [SMILES: CCc1c(F)ccc2cc(O)cc(-c3c(F)c4nc(OCC5(CN6CCC(F)(F)CC6)CC5)nc(N5CCOCC(C)(O)C5)c4c4cn(C)nc34)c12] | 723.6 [M + H]⁺ (Method 1) | 1H), 8.52 (d, J = 4.0 Hz, 1H), 7.80-7.74 (m, 1H), 7.36-7.30 (m, 2H), 7.02- 6.98 (m, 1H), 5.30-5.26 (m, 1H), 4.34 (q, J = 10.9 Hz, 2H), 4.09 (s, 3H), 4.02- 3.70 (m, 6H), 3.56-3.40 (m, 2H), 2.43-2.35 (m, 3H), 2.23- 2.08 (m, 1H), 1.93-1.80 (m, 4H), 1.01 (s, 2H), 0.84 (s, 1H), 0.70- |
| | Example 229 | | 0.65 (m, 2H), |
| | 4-[7-[[1-[(4,4-difluoro-1- | | 0.57 (t, J = 7.3 Hz, |
| | piperidyl)methyl]cyclopropyl] | | 3H), 0.46-0.40 |
| | methoxy]-4-(8-ethyl-7-fluoro-3- | | (m, 2H). |
| | hydroxy-1-naphthyl)-5-fluoro-2- | | |
| | methyl-pyrazolo[4,3-f]quinazolin- | | |
| | 9-yl]-6-methyl-1,4-oxazepan-6-ol | | |
| Intermediate I-143 and | Example 231 | 3.12 min, | 9.86-9.84 (d, J = |
| 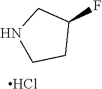 (3S)-fluoropyrrolidine hydrochloride | 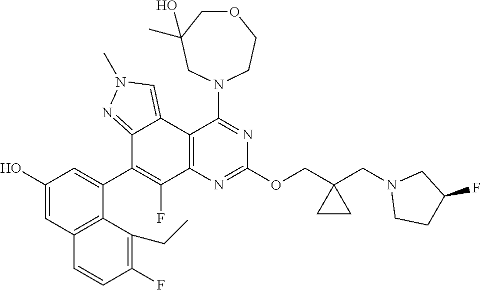 [SMILES: CCc1c(F)ccc2cc(O)cc(-c3c(F)c4nc(OCC5(CN6CC[C@H](F)C6)CC5)nc(N5CCOCC(C)(O)C5)c4c4cn(C)nc34)c12] | 691.6 [M + H]⁺ (Method 1) | 5.60 Hz, 1H), 8.51 (d, J = 3.40 Hz, 1H), 7.79-7.75 (m, 1H), 7.35- 7.31 (m, 2H), 7.01 (d, J = 2.64 Hz, 1H), 5.32- 5.29 (m, 1H), 5.23-5.06 (td, J = 55.67, 5.95 Hz, 1H), 4.36-4.20 (m, 2H), 4.07 (s, 3H), 3.94-3.70 (m, 6H), 3.56- 3.40 (m, 2H), 2.88-2.75 (m, 2H), 2.61-2.57(m, 1H), 2.44-2.29 |
| | Example 231 | | (m, 4H), 2.17- |
| | 4-[4-(8-ethyl-7-fluoro-3-hydroxy- | | 2.01 (m, 2H), |
| | 1-naphthyl)-5-fluoro-2-methyl-7- | | 1.88-1.76 (m, |
| | [[1-[[(3S)-3-fluoropyrrolidin-1- | | 1H), 1.01 (s, 2H), |
| | yl]methyl]cyclopropyl]methoxy] | | 0.84 (s, 1H), 0.63 |
| | pyrazolo[4,3-f]quinazolin-9-yl]-6- | | (m, 2H), 0.58- |
| | methyl-1,4-oxazepan-6-ol | | 0.54 (t, J = 7.44 |
| | | | Hz, 3H), 0.43 (s, |
| | | | 2H). |
| Intermediate I-143 and | Example 237 | 3.19 and | 9.86 (br s, 1H), |
| 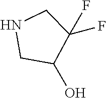 4,4-difluoropyrrolidin- 3-ol | 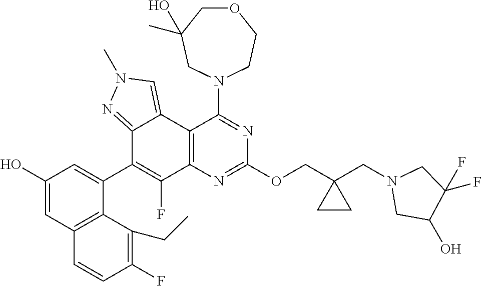 [SMILES: CCc1c(F)ccc2cc(O)cc(-c3c(F)c4nc(OCC5(CN6CC(O)C(F)(F)C6)CC5)nc(N5CCOCC(C)(O)C5)c4c4cn(C)nc34)c12] | 3.22 min, 725.5 [M + H]⁺ (Method 1) | 8.51 (d, J = 3.5 Hz, 1H), 7.80- 7.74 (m, 1H), 7.36-7.30 (m, 2H), 7.01 (d, J = 2.8 Hz, 1H), 5.64 (br d, J = 6.0 Hz, 1H), 5.30-5.26 (m, 1H), 4.36- 4.27 (m, 1H), 4.26-4.16 (m, 1H), 4.08 (s, 3H), 3.98-3.69 (m, 6H), 3.56-3.40 (m, 2H), 3.25- 3.17 (m, 2H), 2.21-2.10 (m, 1H), 1.01 (br s, |
| | Example 237 | | 2H), 0.84 (br s, |
| | 4-[7-[[1-[(3,3-difluoro-4-hydroxy- | | 1H), 0.64 (br s, |
| | pyrrolidin-1- | | 2H), 0.57 (t, J = |
| | yl)methyl]cyclopropyl]methoxy]- | | 7.3 Hz, 3H), 0.47- |
| | 4-(8-ethyl-7-fluoro-3-hydroxy-1- | | 0.39 (m, 2H). |
| | naphthyl)-5-fluoro-2-methyl- | | |
| | pyrazolo[4,3-f]quinazolin-9-yl]-6- | | |
| | methyl-1,4-oxazepan-6-ol | | |
| Intermediate I-143 and | Example 238 | 3.06 min, | 9.87-9.84 (m, |
| 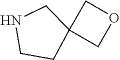 2-oxa-6- azaspiro[3.4]octane | 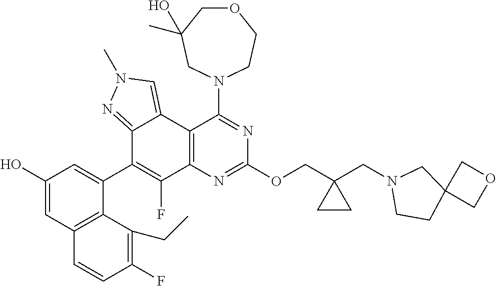 [SMILES: CCc1c(F)ccc2cc(O)cc(-c3c(F)c4nc(OCC5(CN6CCC7(COC7)C6)CC5)nc(N5CCOCC(C)(O)C5)c4c4cn(C)nc34)c12] | 715.6 [M + H]⁺ (Method 1) | 1H), 8.53-8.50 (m, 1H), 7.80- 7.74 (m, 1H), 7.36-7.30 (m, 2H), 7.02-7.00 (m, 1H), 5.28- 5.25 (m, 1H), 4.45-4.38 (m, 4H), 4.33-4.25 (m, 2H), 4.08 (s, 3H), 4.00-3.69 (m, 6H), 3.56- 3.39 (m, 2H), 2.75 (br s, 2H), 2.21-2.10 (m, 1H), 1.99-1.93 (m, 2H), 1.01 (s, 2H), 0.83 (br s, |
| | Example 238 | | 1H), 0.67-0.61 |
| | 4-[4-(8-ethyl-7-fluoro-3-hydroxy- | | (m, 2H), 0.56 (t, |
| | 1-naphthyl)-5-fluoro-2-methyl-7- | | J = 7.5 Hz, 3H), |
| | [[1-(2-oxa-7-azaspiro[3.4]octan- | | 0.46-0.39 (m, |
| | 7- | | 2H). Several H |
| | ylmethyl)cyclopropyl]methoxy] | | overlap with |
| | pyrazolo[4,3-f]quinazolin-9-yl]-6- | | DMSO peak. |
| | methyl-1,4-oxazepan-6-ol | | |
| Intermediate I-143 and | Example 246 | 3.16 min, | 9.90-9.82 (1H, |
| 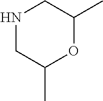 2,6- dimethylmorpholine | 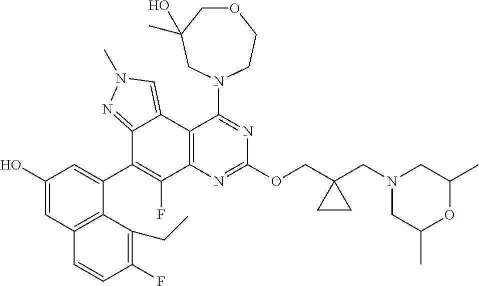 [SMILES: CCc1c(F)ccc2cc(O)cc(-c3c(F)c4nc(OCC5(CN6CC(C)OC(C)C6)CC5)nc(N5CCOCC(C)(O)C5)c4c4cn(C)nc34)c12] | 717.7 [M + H]⁺ (Method 1) | m), 8.55-8.48 (1H, m), 7.81- 7.74 (1H, m), 7.37-7.30 (2H, m), 7.03-6.99 (1H, m), 5.38- 5.24 (1H, m), 4.57-3.38 (15H, m), 2.84-2.74 (1H, m), 2.45- 2.34 (3H, m), 2.32-1.94 (4H, m), 1.60-1.46 (1H, m), 1.06- 0.96 (7H, m), 0.87-0.79 (1H, m), 0.70-0.61 (2H, m), 0.60- |
| | Example 246 | | 0.52 (3H, m), |
| | 4-[7-[[1-[(2,6-dimethylmorpholin- | | 0.47-0.33 (2H, m) |
| | 4- | | |
| | yl)methyl]cyclopropyl]methoxy]- | | |
| | 4-(8-ethyl-7-fluoro-3-hydroxy-1- | | |
| | naphthyl)-5-fluoro-2-methyl- | | |
| | pyrazolo[4,3-f]quinazolin-9-yl]-6- | | |
| | methyl-1,4-oxazepan-6-ol | | |

### Example 58, 6-hydroxy-8-[2-methyl-9-(1,4-oxazepan-4-yl)-7-[[(2R,8S)-2-fluoro-1,2,3,5,6,7-hexahydropyrrolizin-8-yl]methoxy]pyrazolo[4,3-f]quinazolin-4-yl]tetralin-1-one

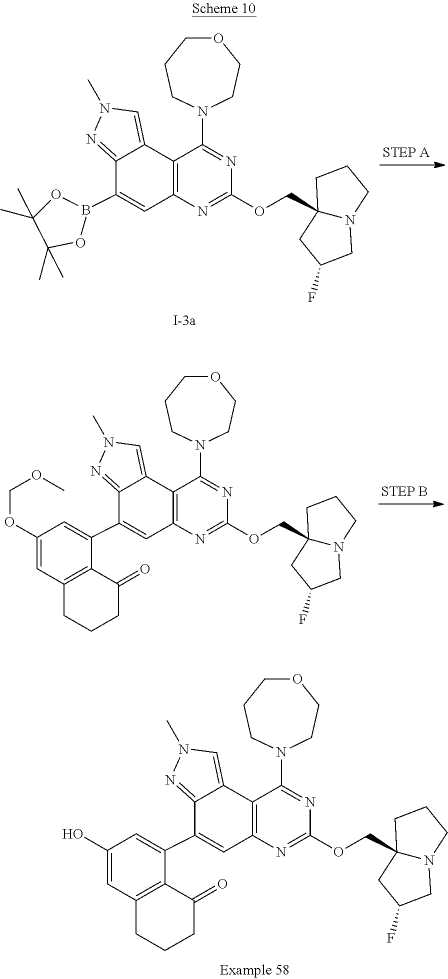 [SMILES: Cn1cc2c(n1)c(B1OC(C)(C)C(C)(C)O1)cc1nc(OC[C@@]34CCCN3C[C@H](F)C4)nc(N3CCCOCC3)c12 ; COCOc1cc2c(c(-c3cc4nc(OC[C@@]56CCCN5C[C@H](F)C6)nc(N5CCCOCC5)c4c4cn(C)nc34)c1)C(=O)CCC2 ; Cn1cc2c(n1)c(-c1cc(O)cc3c1C(=O)CCC3)cc1nc(OC[C@@]34CCCN3C[C@H](F)C4)nc(N3CCCOCC3)c12]

Step A, 8-[7-[[(2R,8S)-2-fluoro-1,2,3,5,6,7-hexahydropyrrolizin-8-yl]methoxy]-2-methyl-9-(1,4-oxazepan-4-yl)pyrazolo[4,3-f]quinazolin-4-yl]-6-(methoxymethoxy)tetralin-1-one. Added Potassium carbonate (20.36 mg, 0.15 mmol) to a stirring solution of 4-[7-[[(2R,8S)-2-fluoro-1,2,3,5,6,7-hexahydropyrrolizin-8-yl]methoxy]-2-methyl-4-(4,4,5,5-tetramethyl-1,3,2-dioxaborolan-2-yl)pyrazolo[4,3-f]quinazolin-9-yl]-1,4-oxazepane (27.81 mg, 0.05 mmol) and 8-bromo-6-(methoxymethoxy)tetralin-1-one (A-13) (14. mg, 0.05 mmol) in 1,4-Dioxane (0.81 mL) and Water (0.16 mL). The solution was degassed with N₂, and [1,1′-Bis(diphenylphosphino)ferrocene]Palladium(II) chloride dichloromethane complex (4.01 mg) added. The mixture was heated to 100° C. for 3 h. Additional [1,1′-Bis(diphenylphosphino)ferrocene]Palladium(II) chloride dichloromethane complex (4 mg) was added. Reaction mixture allowed to stir at 100° C. overnight. The reaction was cooled to room temperature. Diluted with ethyl acetate (10 mL), filtered over a plug of celite, plug washed with ethyl acetate (2×10 mL) and concentrated under reduced pressure. Purification by flash column chromatography on KP-amino D eluting with 0-100% ethyl acetate in petrol afforded 8-[7-[[(2R,8S)-2-fluoro-1,2,3,5,6,7-hexahydropyrrolizin-8-yl]methoxy]-2-methyl-9-(1,4-oxazepan-4-yl)pyrazolo[4,3-f]quinazolin-4-yl]-6-(methoxymethoxy)tetralin-1-one (25.1 mg, 0.0389 mmol, 79% yield) as an off-white solid.

UPLC-MS (ES⁺, Method 5): 1.52 min, 645.3 [M+H]⁺.

Step B, 6-hydroxy-8-[2-methyl-9-(1,4-oxazepan-4-yl)-7-[[(2R,8S)-2-fluoro-1,2,3,5,6,7-hexahydropyrrolizin-8-yl]methoxy]pyrazolo[4,3-f]quinazolin-4-yl]tetralin-1-one. Trifluoroacetic acid (0.15 mL, 1.95 mmol) was added to a solution of 6-(methoxymethoxy)-8-[2-methyl-9-(1,4-oxazepan-4-yl)-7-[[(2R,8S)-2-fluoro-1,2,3,5,6,7-hexahydropyrrolizin-8-yl]methoxy]pyrazolo[4,3-f]quinazolin-4-yl]tetralin-1-one (25.1 mg, 0.04 mmol) and

Triethylsilane (0.06 mL, 0.39 mmol) in DCM (0.3893 mL). Afterwards, allowed to stir at room temperature for 1 h. Additional Triethylsilane (0.03 mL, 0.19 mmol) and Trifluoroacetic acid (0.07 mL, 0.97 mmol) were added. Reaction mixture allowed to stir at room temperature for 1 h. The reaction mixture was concentrated under reduced pressure and purified by reverse phase column chromatography (eluting with 5-30% MeCN (0.1% formic acid) in Water (0.1% formic acid)). Product containing fractions were isolated by SCX (MeCN wash (×2) followed by 1 M NH₃ in MeOH (×2)) to afford the desired product 6-hydroxy-8-[2-methyl-9-(1,4-oxazepan-4-yl)-7-[[(2R,8S)-2-fluoro-1,2,3,5,6,7-hexahydropyrrolizin-8-yl]methoxy]pyrazolo[4,3-f]quinazolin-4-yl]tetralin-1-one (6.5 mg, 0.0108 mmol, 28% yield) as a yellow solid.

UPLC-MS (ES⁺, Method 1): 2.22 min, 601.5 [M+H]⁺.

¹H NMR (400 MHz, DMSO-d6) δ/ppm: 10.38 (s, 1H), 8.35 (s, 1H), 7.03 (s, 1H), 6.76 (d, J=2.4 Hz, 1H), 6.59 (d, J=2.5 Hz, 1H), 5.40-5.18 (m, 1H), 4.16-3.98 (m, 5H), 3.88-3.69 (m, 8H), 3.18-2.99 (m, 3H), 2.99-2.89 (m, 2H), 2.89-2.78 (m, 1H), 2.44-2.33 (m, 2H), 2.22-1.93 (m, 7H), 1.91-1.71 (m, 3H).

The following examples in table 25 were made by analogy with example 58 (scheme 10) starting from the intermediate and building blocks for step A as described in the table.

TABLE 25

| | | UPLC-MS | |
|---|---|---|---|
| Step A, intermediate + | | (ES⁺): min, | ¹H NMR (400 MHz, |
| building block | Example Structure/Name | m/z | DMSO-d₆) δ/ppm |
| Intermediate I-3a and | Example 66 | 2.93 min, | 9.12 (s, 1H), 8.44 |
| 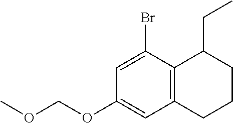 A-14 8-bromo-1-ethyl-6- (methoxymethoxy) tetralin | 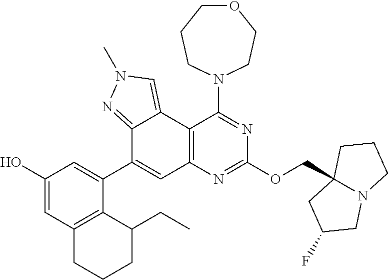 [SMILES: CCC1CCCc2cc(O)cc(-c3cc4nc(OC[C@@]56CCCN5C[C@H](F)C6)nc(N5CCCOCC5)c4c4cn(C)nc34)c21] | 615.3 [M + H]⁺ (Method 1) | (s, 1H), 7.16 (s, 1H), 6.61-6.42 (m, 2H), 5.41-5.16 (m, 1H), 4.21-3.95 (m, 5H), 3.93-3.65 (m, 8H), 3.19-3.00 (m, 3H), 2.90-2.78 (m, 1H), 2.78-2.67 (m, 2H), 2.57-2.45 (m, 1H), 2.24-1.91 (m, 5H), 1.91-1.51 (m, 7H), 1.24-1.01 (m, 2H), 0.27 (t, J = 7.1 Hz, 3H). 1H obscured by DMSO peak. |
| | Example 66 | | |
| | 1-ethyl-8-[2-methyl-9-(1,4- | | |
| | oxazepan-4-yl)-7-[[(2R,8S)-2- | | |
| | fluoro-1,2,3,5,6,7- | | |
| | hexahydropyrrolizin-8- | | |
| | yl]methoxy]pyrazolo[4,3- | | |
| | f]quinazolin-4-yl]tetralin-6-ol | | |

### Example 61, 5-[4-(4-fluoro-3-hydroxy-1-naphthyl)-2-methyl-7-[[(2R,8S)-2-fluoro-1,2,3,5,6,7-hexahydropyrrolizin-8-yl]methoxy]pyrazolo[4,3-f]quinazolin-9-yl]-N,N-dimethyl-4,6,7,8-tetrahydropyrazolo[1,5-a][1,4]diazepine-2-carboxamide

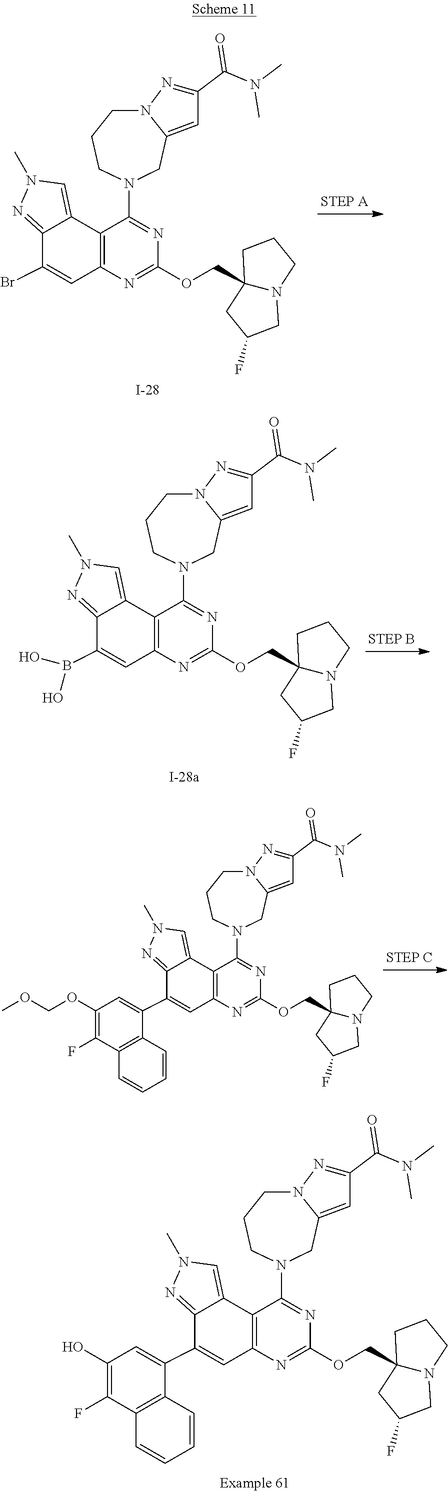

Step A, [9-[2-(dimethylcarbamoyl)-4,6,7,8-tetrahydropyrazolo[1,5-a][1,4]diazepin-5-yl]-7-[[(2R,8S)-2-fluoro-1,2,3,5,6,7-hexahydropyrrolizin-8-yl]methoxy]-2-methyl-pyrazolo[4,3-f]quinazolin-4-yl]boronic acid (I-28a). 5-[4-bromo-7-[(2R,8S)-2-fluoro-1,2,3,5,6,7-hexahydropyrrolizin-8-yl]methoxy]-2-methyl-pyrazolo[4,3-f]quinazolin-9-yl]-N,N-dimethyl-4,6,7,8-tetrahydropyrazolo[1,5-a][1,4]diazepine-2-carboxamide (298. mg, 0.48 mmol), potassium acetate (140.04 mg, 1.43 mmol), bis(pinacolato)diboron (603.9 mg, 2.38 mmol) and 2-dicyclohexylphosphino-2′,4′,6′-triisopropylbiphenyl (226.74 mg, 0.48 mmol) was added to a 20 ml microwave vial. The reaction vessel was sealed and vacuum cycled with nitrogen. Tris(dibenzylideneacetone)dipalladium (0) (87.11 mg, 0.1 mmol) was added in one portion and the reaction was resealed and vacuum cycled. The reaction was heated to 75° C. overnight. The reaction was filtered through a celite pad, washing with EtOAc afford the desired product [9-[2-(dimethylcarbamoyl)-4,6,7,8-tetrahydropyrazolo[1,5-a][1,4]diazepin-5-yl]-7-[[(2R,8S)-2-fluoro-1,2,3,5,6,7-hexahydropyrrolizin-8-yl]methoxy]-2-methyl-pyrazolo[4,3-f]quinazolin-4-yl]boronic acid (281 mg, 0.4751 mmol, 100% yield) as a brown solid.

UPLC-MS (ES⁺, Method 5): 1.18 min, 592.3 [M+H]⁺. (Boronic acid fragment)

Step B, 5-[4-[4-fluoro-3-(methoxymethoxy)-1-naphthyl]-2-methyl-7-[(2R,8S)-2-fluoro-1,2,3,5,6,7-hexahydropyrrolizin-8-yl]methoxy]pyrazolo[4,3-f]quinazolin-9-yl]-N,N-dimethyl-4,6,7,8-tetrahydropyrazolo[1,5-a][1,4]diazepine-2-carboxamide. A microwave vial was charged with 4-bromo-1-fluoro-2-(methoxymethoxy) naphthalene (A-16) (55. mg, 0.19 mmol), N,N-dimethyl-5-[2-methyl-7-[(2R,8S)-2-fluoro-1,2,3,5,6,7-hexahydropyrrolizin-8-yl]methoxy]-4-(4,4,5,5-tetramethyl-1,3,2-dioxaborolan-2-yl)pyrazolo[4,3-f]quinazolin-9-yl]-4,6,7,8-tetrahydropyrazolo[1,5-a][1,4]diazepine-2-carboxamide (185.63 mg, 0.19 mmol), Bis(triphenylphosphine) palladium(II) dichloride (9.59 mg, 0.01 mmol) and sodium carbonate (28.96 mg, 0.27 mmol) in 1,4-Dioxane (0.8 mL) and Water (0.4 mL). The vial was degassed with nitrogen, sealed and the mixture irradiated in the microwave at 120° C. for 2 hours. The mixture was reduced in vacuo, taken up in EtOAc and filtered through a phase separating filter. The filtrate was concentrated and the resulting crude was purified by flash column chromatography (12 g KP-NH column, eluting in 0-100% EtOAc in Pet. Ether, followed by 0-4% MeOH in DCM) to afford the desired product 5-[4-[4-fluoro-3-(methoxymethoxy)-1-naphthyl]-2-methyl-7-[[(2R,8S)-2-fluoro-1,2,3,5,6,7-hexahydropyrrolizin-8-yl]methoxy]pyrazolo[4,3-f]quinazolin-9-yl]-N, N-dimethyl-4,6,7,8-tetrahydropyrazolo[1,5-a][1,4]diazepine-2-carboxamide (74 mg, 0.0984 mmol, 51% yield) as a light yellow oil.

UPLC-MS (ES⁺, Method 5): 1.67 min, 752.4 [M+H]⁺.

Step C, 5-[4-(4-fluoro-3-hydroxy-1-naphthyl)-2-methyl-7-[[(2R,8S)-2-fluoro-1,2,3,5,6,7-hexahydropyrrolizin-8-yl]methoxy]pyrazolo[4,3-f]quinazolin-9-yl]-N,N-dimethyl-4,6,7,8-tetrahydropyrazolo[1,5-a][1,4]diazepine-2-carboxamide. Trifluoroacetic acid (0.19 mL, 2.46 mmol) was added to a solution of 5-[4-[4-fluoro-3-(methoxymethoxy)-1-naphthyl]-2-methyl-7-[[(2R,8S)-2-fluoro-1,2,3,5,6,7-hexahydropyrrolizin-8-yl]methoxy]pyrazolo[4,3-f]quinazolin-9-yl]-N,N-dimethyl-4,6,7,8-tetrahydropyrazolo[1,5-a][1,4]diazepine-2-carboxamide (74. mg, 0.1 mmol) and Triethylsilane (0.08 mL, 0.49 mmol) in DCM (0.9 mL) and the mixture stirred at room temperature for 30 min. The reaction mixture was loaded onto an SCX-2 column, washed with 2 column volumes (CV) of MeOH, eluted with 2 CV 1N NH₃/MeOH and the solvents removed in vacuo. The crude material was purified by reverse phase flash column chromatography (20 g C18-column, eluting int 20-50% MeCN in Water, 0.1% FA). Product containing fractions were loaded onto a pre-equilibrated SCX-2 column, washed with 2 CV MeOH, and eluted with 2 CV 1N NH₃/MeOH to afford the desired product 5-[4-(4-fluoro-3-hydroxy-1-naphthyl)-2-methyl-7-[[(2R,8S)-2-fluoro-1,2,3,5,6,7-hexahydropyrrolizin-8-yl]methoxy]pyrazolo[4,3-f]quinazolin-9-yl]-N,N-dimethyl-4,6,7,8-tetrahydropyrazolo[1,5-a][1,4]diazepine-2-carboxamide (6.1 mg, 0.0086 mmol, 9% yield) as a yellow solid.

UPLC-MS (ES⁺, Method 1): 2.88 min, 708.6 [M+H]⁺.

¹H NMR (400 MHz, DMSO-d6) δ/ppm: 10.24 (s, 1H), 8.50 (s, 1H), 8.00 (d, J=8.3 Hz, 1H), 7.59-7.51 (m, 2H), 7.39-7.35 (m, 2H), 7.30-7.25 (m, 1H), 6.45 (s, 1H), 5.29 (d, J=54.7 Hz, 1H), 4.85 (s, 2H), 4.56-4.43 (m, 2H), 4.10 (s, 3H), 4.08-3.89 (m, 4H), 3.27 (s, 3H), 3.21-2.99 (m, 3H), 2.94 (s, 3H), 2.91-2.77 (m, 1H), 2.22-1.94 (m, 5H), 1.90-1.71 (m, 3H).

The following examples in table 26 were made by analogy with example 61 (scheme 11) starting from the intermediate and building blocks for step B as described in the table. Step C was not required where there was no protecting group present on the step B coupling partner.

TABLE 26

| | | UPLC-MS | |
|---|---|---|---|
| Step B, intermediate + | | (ES⁺): min, | ¹H NMR (400 MHz, |
| building block | Example Structure/Name | m/z | DMSO-d₆) δ/ppm |
| Intermediate I-28a and | Example 63 | 3.65 min, | 8.49 (s, 1H), 8.08 |
| 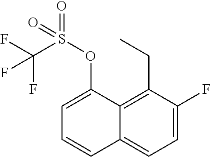 A-15 8-ethyl-7- fluoronapthalen-1-yl trifluoromethane- sulfonate | 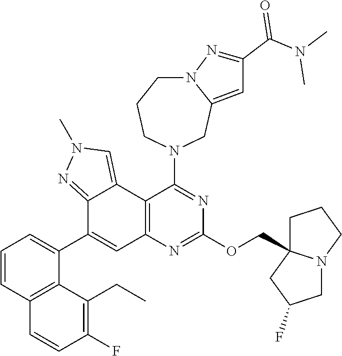 [SMILES: CCc1c(F)ccc2cccc(-c3cc4nc(OC[C@@]56CCCN5C[C@H](F)C6)nc(N5CCCn6nc(C(=O)N(C)C)cc6C5)c4c4cn(C)nc34)c12] | 720.4 [M + H]⁺ (Method 6) | (dd, J = 1.4, 8.5 Hz, 1H), 8.01 (dd, J = 6.1, 8.8 Hz, 1H), 7.55 (t, J = 7.8 Hz, 1H) 7.46 (t, J = 8.9 Hz, 2H), 7.38 (s, 1H), 6.24 (s, 1H), 5.27 (d, J = 54.3 Hz, 1H), 4.96-4.78 (m, 2H), 4.56-4.47 (m, 2H), 4.06-3.85 (m, 7H), 3.26 (s, 3H), 3.10-2.83 (m, 7H), 2.21-1.75 (m, 10H), 0.58 (t, J = 7.2 Hz, 3H) |
| | Example 63 | | |
| | 5-[4-(8-ethyl-7-fluoro-1- | | |
| | naphthyl)-2-methyl-7- | | |
| | [[(2R,8S)-2-fluoro-1,2,3,5,6,7- | | |
| | hexahydropyrrolizin-8- | | |
| | yl]methoxy]pyrazolo[4,3- | | |
| | f]quinazolin-9-yl]-N,N-dimethyl- | | |
| | 4,6,7,8-tetrahydropyrazolo[1,5- | | |
| | a][1,4]diazepine-2- | | |
| | carboxamide | | |
| Intermediate I-28a and | Example 64 | 3.18 min, | 10.43 (s, 1H), 8.45 |
| 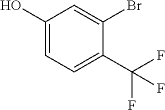 [SMILES: Oc1ccc(C(F)(F)F)c(Br)c1] 3-Bromo-4- (trifluoromethyl)phenol | 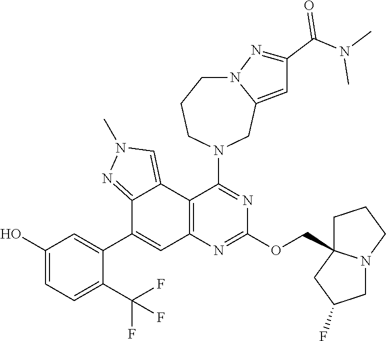 [SMILES: CN(C)C(=O)c1cc2n(n1)CCCN(c1nc(OC[C@@]34CCCN3C[C@H](F)C4)nc3cc(-c4cc(O)ccc4C(F)(F)F)c4nn(C)cc4c13)C2] | 708.3 [M + H]⁺ (Method 1) | (s, 1H), 7.69 (s, J = 8.8 Hz, 1H), 7.21 (s, 1H), 7.01 (dd, J = 1.8, 8.8 Hz, 1H), 6.87 (d, J = 2.4 Hz, 1H), 6.36 (s, 1H), 5.26 (d, J = 53.4 Hz, 1H), 4.81 (s, 2H), 4.49-4.47 (m, 2H), 4.15 (s, 3H), 3.02-3.89 (m, 4H), 3.26 (s, 3H), 3.09- 2.81 (m, 7H), 2.11- 1.97 (m, 6H), 1.83- 1.78 (m, 2H). |
| | Example 64 | | |
| | 5-[4-[5-hydroxy-2- | | |
| | (trifluoromethyl)phenyl]-2- | | |
| | methyl-7-[[rac-(2R,8S)-2- | | |
| | fluoro-1,2,3,5,6,7- | | |
| | hexahydropyrrolizin-8- | | |
| | yl]methoxy]pyrazolo[4,3- | | |
| | f]quinazolin-9-yl]-N,N-dimethyl- | | |
| | 4,6,7,8-tetrahydropyrazolo[1,5- | | |
| | a][1,4]diazepine-2- | | |
| | carboxamide | | |
| Intermediate I-28a and | Example 77 | 2.68 min, | 8.41 (s, 1H), 7.11 |
| 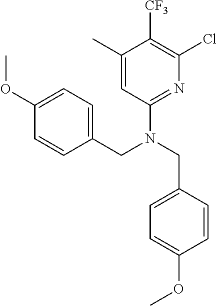 6-chloro-N,N-bis[(4- methoxyphenyl) | 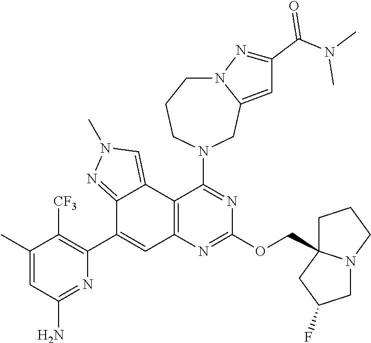 [SMILES: Cc1cc(N)nc(-c2cc3nc(OC[C@@]45CCCN4C[C@H](F)C5)nc(N4CCCn5nc(C(=O)N(C)C)cc5C4)c3c3cn(C)nc23)c1C(F)(F)F] | 722.7 [M + H]⁺ (Method 1) | (s, 1H), 6.68 (s, 2H), 6.47 (s, 1H), 6.31 (s, 1H), 5.26 (d, J = 54.0 Hz, 1H), 4.88-4.74 (m, 2H), 4.48 (t, J = 5.2 Hz, 2H), 4.14 (s, 3H), 3.99-3.88 (m, 3H), 3.26 (s, 3H), 3.10- 3.04 (m, 2H), 3.00- 2.98 (m, 1H), 2.93 (s, 3H), 2.84-2.80 (m, 1H), 2.39 (s, 3H), 2.15-2.08 (m, 3H), 2.03-1.96 (m, 3H), 1.84-1.74 (m, 3H). |
| methyl]-4-methyl-5- | Example 77 | | |
| (trifluoromethyl) | 5-[4-[6-amino-4-methyl-3- | | |
| pyridin-2-amine (A-4) | (trifluoromethyl)-2-pyridyl]-7- | | |
| | [[(2R,8S)-2-fluoro-1,2,3,5,6,7- | | |
| | hexahydropyrrolizin-8- | | |
| | cyl]methoxy]-2-methyl- | | |
| | pyrazolo[4,3-f]quinazolin-9-yl]- | | |
| | N,N-dimethyl-4,6,7,8- | | |
| | tetrahydropyrazolo[1,5- | | |
| | a][1,4]diazepine-2- | | |
| | carboxamide | | |
| Intermediate I-28a and | Example 81 | 3.20 min, | 8.47 (s, 1H), 7.42- |
| 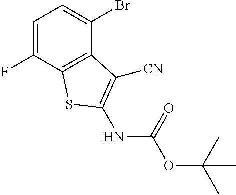 tert-butyl N-(4-bromo- 3-cyano-7-fluoro- benzothiophen-2- yl)carbamate | 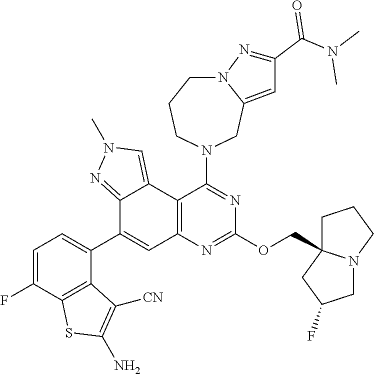 [SMILES: CN(C)C(=O)c1cc2n(n1)CCCN(c1nc(OC[C@@]34CCCN3C[C@H](F)C4)nc3cc(-c4ccc(F)c5sc(N)c(C#N)c45)c4nn(C)cc4c13)C2] | 738.3 [M + H]⁺ (Method 6) | 7.39 (m, 2H), 7.09- 7.04 (m, 1H), 6.48 (s, 1H), 4.86 (d, J = 53.48 Hz, 1H), 5.05-4.98 (m, 1H), 4.60 (s, 1H), 4.55- 4.52 (m, 3H), 4.24- 4.16 (m, 7H), 4.09- 3.96 (m, 2H), 3.09- 3.06 (m, 5H), 2.36- 1.94 (m, 11H), 0.92 (t, J = 7.4 Hz, 1H). |
| | Example 81 | | |
| | 5-[4-(2-amino-3-cyano-7- | | |
| | fluoro-benzothiophen-4-yl)-7- | | |
| | [[(2R,8S)-2-fluoro-1,2,3,5,6,7- | | |
| | hexahydropyrrolizin-8- | | |
| | yl]methoxy]-2-methyl- | | |
| | pyrazolo[4,3-f]quinazolin-9-yl]- | | |
| | N,N-dimethyl-4,6,7,8- | | |
| | tetrahydropyrazolo[1,5- | | |
| | a][1,4]diazepine-2- | | |
| | carboxamide | | |
| Intermediate I-28a and | Example 83 | 2.90 min, | 14.47 (s, 1H), 8.54 |
| 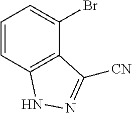 [SMILES: N#Cc1n[nH]c2cccc(Br)c12] 4-bromo-1H-indazole- 3-carbonitrile | 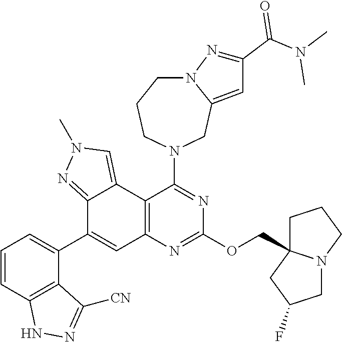 [SMILES: CN(C)C(=O)c1cc2n(n1)CCCN(c1nc(OC[C@@]34CCCN3C[C@H](F)C4)nc3cc(-c4cccc5[nH]nc(C#N)c45)c4nn(C)cc4c13)C2] | 689.3 [M + H]⁺ (Method 6) | (s, 1H), 7.90-7.85 (m, 1H), 7.72-7.76 (m, 1H), 7.63-7.58 (m, 1H), 7.47 (s, 1H), 6.24 (s, 1H), 5.37-5.18 (m, 1H), 4.87 (s, 2H), 4.56- 4.72 (m, 2H), 4.13 (s, 3H), 4.09-4.03 (m, 1H), 4.02-3.93 (m, 3H), 3.23 (s, 3H), 3.14-3.07 (m, 2H), 3.04-2.99 (m, 1H), 2.91 (s, 3H), 2.88-2.79 (m, 1H), 2.21-1.97 (m, 5H), 1.88-1.72 (m, 3H). |
| | Example 83 | | |
| | 5-[4-(3-cyano-1H-indazol-4-yl)- | | |
| | 2-methyl-7-[[(2R,8S)-2-fluoro- | | |
| | 1,2,3,5,6,7- | | |
| | hexahydropyrrolizin-8- | | |
| | yl]methoxy]pyrazolo[4,3- | | |
| | f]quinazolin-9-yl]-N,N-dimethyl- | | |
| | 4,6,7,8-tetrahydropyrazolo[1,5- | | |
| | a][1,4]diazepine-2- | | |
| | carboxamide | | |
| Intermediate I-28a and | Example 84 | 3.32 min, | 13.42 (s, 1H), 8.47 |
| 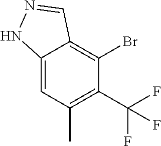 A-24 4-bromo-6-methyl-5- (trifluoromethyl)-1H- indazole (A-24) | 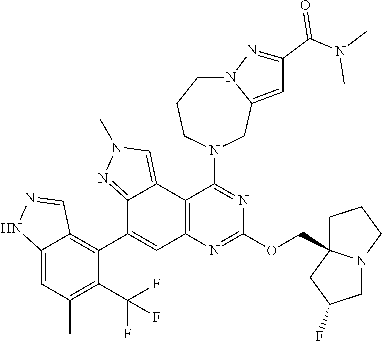 [SMILES: Cc1cc2[nH]ncc2c(-c2cc3nc(OC[C@@]45CCCN4C[C@H](F)C5)nc(N4CCCn5nc(C(=O)N(C)C)cc5C4)c3c3cn(C)nc23)c1C(F)(F)F] | 746.4 [M + H]+ (Method 6) | (s, 1H), 7.69 (s, 1H), 7.46 (s, 1H), 7.28 (s, 1H), 6.39 (s, 1H), 4.85 (d, J = 5.4 Hz, 2H), 4.51 (s, 2H), 4.09 (s, 3H), 4.04-3.80 (m, 3H), 3.27 (s, 3H), 2.94 (s, 3H), 2.67 (s, 3H), 2.47- 2.25 (m, 5H), 2.24- 1.66 (m, 9H). |
| | Example 84 | | |
| | 5-[7-[[(2R,8S)-2-fluoro- | | |
| | 1,2,3,5,6,7- | | |
| | hexahydropyrrolizin-8- | | |
| | yl]methoxy]-2-methyl-4-[6- | | |
| | methyl-5-(trifluoromethyl)-1H- | | |
| | indazol-4-yl]pyrazolo[4,3- | | |
| | f]quinazolin-9-yl]-N,N-dimethyl- | | |
| | 4,6,7,8-tetrahydropyrazolo[1,5- | | |
| | a][1,4]diazepine-2- | | |
| | carboxamide | | |

### Example 89, 5-ethyl-6-fluoro-4-[7-[[(2R,8S)-2-fluoro-1,2,3,5,6,7-hexahydropyrrolizin-8-yl]methoxy]-9-[3-(3-hydroxypropyl)-3,8-diazabicyclo[3.2.1]octan-8-yl]-2-methyl-pyrazolo[4,3-f]quinazolin-4-yl]naphthalen-2-ol

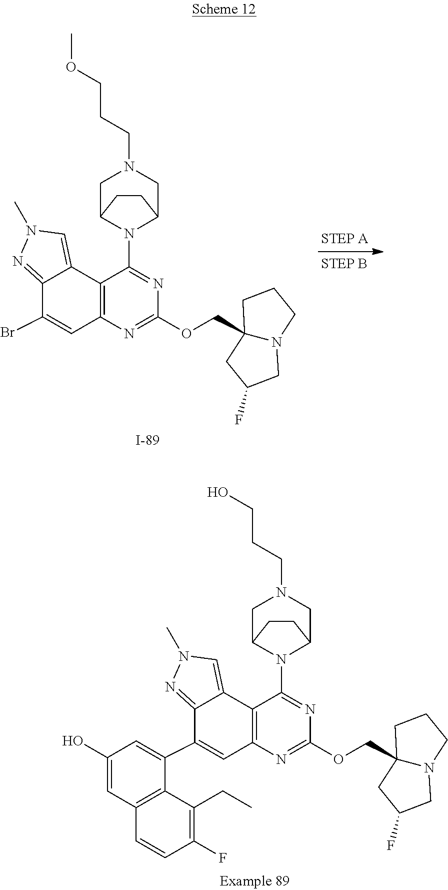

Step A, 4-[8-ethyl-7-fluoro-3-(methoxymethoxy)-1-naphthyl]-7-[[(2R,8S)-2-fluoro-1,2,3,5,6,7-hexahydropyrrolizin-8-yl]methoxy]-9-[3-(3-methoxypropyl)-3,8-diazabicyclo[3.2.1]octan-8-yl]-2-methyl-pyrazolo[4,3-f]quinazoline. A solution of 2-[8-ethyl-7-fluoro-3-(methoxymethoxy)-1-naphthyl]-4,4,5,5-tetramethyl-1,3,2-dioxaborolane (358.7 mg, 1 mmol), 4-bromo-7-[(2R,8S)-2-fluoro-1,2,3,5,6,7-hexahydropyrrolizin-8-yl]methoxy]-9-[3-(3-methoxypropyl)-3,8-diazabicyclo[3.2.1]octan-8-yl]-2-methyl-pyrazolo[4,3-f]quinazoline (500 mg, 0.83 mmol), and Cesium Carbonate (540.74 mg, 1.66 mmol) in 1,4-Dioxane (6.99 mL) and Water (1.31 mL) was degassed for 10 mins. [1,1′-Bis(di-tert-butylphosphino)ferrocene]dichloropalladium(II) (54.08 mg, 0.08 mmol) was added to the solution and heated to 100° C. for 1 hr under N₂. The reaction mixture was cooled to room temperature then filtered through a phase separator cartridge, washing with EtOAc. The filtrate was concentrated under reduced pressure and the crude material was purified by silica gel chromatography eluting 40% EtOAc in petroleum ether. The desired fractions were combined and concentrated under reduced pressure to give 4-[8-ethyl-7-fluoro-3-(methoxymethoxy)-1-naphthyl]-7-[(2R,8S)-2-fluoro-1,2,3,5,6,7-hexahydropyrrolizin-8-yl]methoxy]-9-[3-(3-methoxypropyl)-3,8-diazabicyclo[3.2.1]octan-8-yl]-2-methyl-pyrazolo[4,3-f]quinazoline (140 mg, 0.1852 mmol, 22% yield) as a pale yellow oil.

UPLC-MS (ES⁺, Method 5): 2.35 min, 698.3 [M+H]⁺.

Step B, 5-ethyl-6-fluoro-4-[7-[[(2R,8S)-2-fluoro-1,2,3,5,6,7-hexahydropyrrolizin-8-yl]methoxy]-9-[3-(3-hydroxypropyl)-3,8-diazabicyclo[3.2.1]octan-8-yl]-2-methyl-pyrazolo[4,3-f]quinazolin-4-yl]naphthalen-2-ol (Example 89). To a mixture of 4-[8-ethyl-7-fluoro-3-(methoxymethoxy)-1-naphthyl]-7-[[(2R,8S)-2-fluoro-1,2,3,5,6,7-hexahydropyrrolizin-8-yl]methoxy]-9-[3-(3-methoxypropyl)-3,8-diazabicyclo[3.2.1]octan-8-yl]-2-methyl-pyrazolo[4,3-f]quinazoline (140 mg, 0.19 mmol) in DCM (1.85 mL) was added boron tribromide (109.37 mg, 0.437 mmol) at 25° C. The mixture was stirred at 25° C. for 2 hrs. The reaction mixture was concentrated and purified by reverse phase chromatography (eluting in 0-45% acetonitrile (0.1% formic acid) in water (0.1% formic acid)) and freeze dried to give the final product 5-ethyl-6-fluoro-4-[7-[[(2R,8S)-2-fluoro-1,2,3,5,6,7-hexahydropyrrolizin-8-yl]methoxy]-9-[3-(3-hydroxypropyl)-3,8-diazabicyclo[3.2.1]octan-8-yl]-2-methyl-pyrazolo[4,3-f]quinazolin-4-yl]naphthalen-2-ol (5 mg, 0.007 mmol, 3% yield) as a pale yellow solid.

UPLC-MS (ES⁺, Method 6): 3.47 min, 698.3 [M+H]⁺.

¹H NMR (400 MHz, MeOH-d4) δ/ppm: 8.38 (s, 1H), 7.69 (dd, J=9.0, 5.8 Hz, 1H), 7.44 (s, 1H), 7.30 (d, J=2.7 Hz, 1H), 7.24 (t, J=9.4 Hz, 1H), 7.06 (d, J=2.6 Hz, 1H), 5.64-5.48 (d, J=48.0 Hz, 1H), 4.74 (d, J=13.0 Hz, 2H), 4.69-4.51 (m, 2H), 4.17 (s, 3H), 3.98-3.68 (m, 5H), 3.50-3.34 (m, 1H), 3.04 (d, J=11.1 Hz, 2H), 2.92-2.34 (m, 8H), 2.34-2.22 (m, 2H), 2.22-1.98 (m, 6H), 1.84-1.70 (m, 2H), 1.47-1.20 (m, 2H), 0.62 (t, J=7.3 Hz, 3H).

The following examples in table 27 were made by analogy with example 89 (scheme 12) starting from the intermediate and building blocks for step A as described in the table.

TABLE 27

| | | UPLC-MS | ¹H NMR (400 |
|---|---|---|---|
| Step A Intermediate | | (ES⁺): min, | MHz, DMSO- |
| and building block | Example Structure/Name | m/z | d₆) δ/ppm |
| 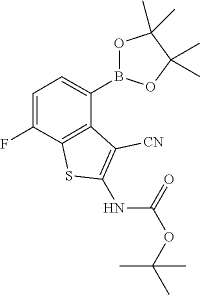 | 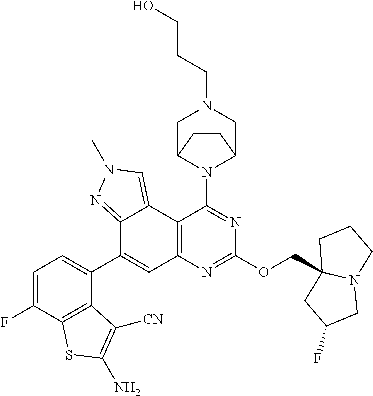 [SMILES: Cn1cc2c(n1)c(-c1ccc(F)c3sc(N)c(C#N)c13)cc1nc(OC[C@@]34CCCN3C[C@H](F)C4)nc(N3C4CCC3CN(CCCO)C4)c12] | 3.12 min, 700.3 [M + H]+ (Method 6) | 8.32 (s, 1H), 7.86 (s, 2H), 7.37-7.34 (m, 1H), 7.25 (s, 1H), 7.14-7.09 (m, 1H), 5.35- 5.21 (d, 1H), 4.51-4.45 (m, 3H), 4.14 (s, 3H), 4.11-4.09 (m, 1H), 4.03- 4.00 (m, 1H), 3.11-3.07 (m, 4H), 3.01 (s, 1H), 2.84-2.77 (m, 4H), 2.16- 2.02 (m, 4H), 1.86-1.77 (m, 7H), 1.61-1.58 (m, 2H), 1.54 (s, 1H) Exchangeable proton not seen. |
| Intermediate 1-89 and | Example 90 | | |
| A-26 | 2-amino-7-fluoro-4-[7-[[(2R,8S)-2- | | |
| tert-butyl N-[3-cyano-7- | fluoro-1,2,3,5,6,7- | | |
| fluoro-4-(4,4,5,5- | hexahydropyrrolizin-8- | | |
| tetramethyl-1,3,2- | yl]methoxy]-9-[3-(3- | | |
| dioxaborolan-2- | hydroxypropyl)-3,8- | | |
| yl)benzothiophen-2- | diazabicyclo[3.2.1]octan-8-yl]-2- | | |
| yl]carbamate (A-26) | methyl-pyrazolo[4,3-f]quinazolin- | | |
| | 4-yl]benzothiophene-3-carbonitrile | | |

### Example 118, 1-[4-(8-ethyl-7-fluoro-3-hydroxy-1-naphthyl)-2-methyl-7-[(2R,8S)-2-fluoro-1,2,3,5,6,7-hexahydropyrrolizin-8-yl]methoxy]pyrazolo[4,3-f]quinazolin-9-yl]-4-methyl-piperidin-4-ol

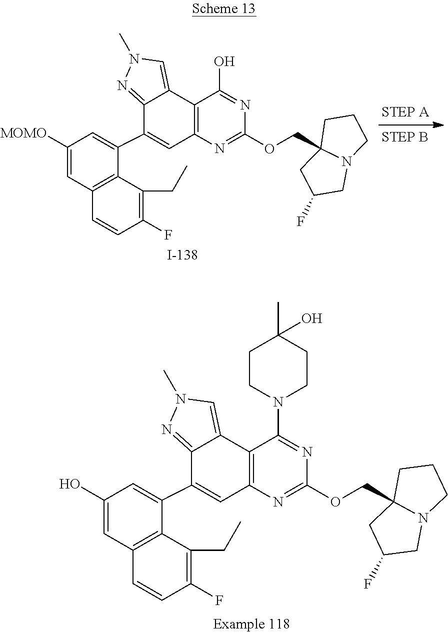

Step A, 1-[4-[8-ethyl-7-fluoro-3-(methoxymethoxy)-1-naphthyl]-2-methyl-7-[[(2R,8S)-2-fluoro-1,2,3,5,6,7-hexahydropyrrolizin-8-yl]methoxy]pyrazolo[4,3-f]quinazolin-9-yl]-4-methyl-piperidin-4-ol. To 4-[8-ethyl-7-fluoro-3-(methoxymethoxy)-1-naphthyl]-2-methyl-7-[[(2R,8S)-2-fluoro-1,2,3,5,6,7-hexahydropyrrolizin-8-yl]methoxy]pyrazolo[4,3-f]quinazolin-9-ol (56.2 mg, 0.1 mmol), (Benzotriazol-1-yloxy)tris(dimethylamino)phosphonium hexafluorophosphate (50.59 mg, 0.11 mmol) and 4-methylpiperidin-4-ol (16.47 mg, 0.14 mmol) in THF (1 mL) was added N,N-Diisopropylethylamine (0.1 mL, 0.57 mmol) and the reaction stirred at 65° C. for 2 h. The reaction mixture was cooled to rt, quenched with sat. aq. NaHCO₃ (20 mL), diluted with EtOAc (15 mL). The phases were separated and the aqueous phase extracted with EtOAc (2×10 mL). The combined organic fractions were dried over Na₂SO₄, filtered and concentrated under reduced pressure. The crude material was purified by silica gel chromatography (11 g KP-amino, 0-100% EtOAc in Pet. ether, then 0-20% MeOH in DCM) the desired fractions were combined and concentrated under reduced pressure to give 1-[4-[8-ethyl-7-fluoro-3-(methoxymethoxy)-1-naphthyl]-2-methyl-7-[[(2R,8S)-2-fluoro-1,2,3,5,6,7-hexahydropyrrolizin-8-yl]methoxy]pyrazolo[4,3-f]quinazolin-9-yl]-4-methyl-piperidin-4-ol (48 mg, 0.0699 mmol, 73% yield) as a yellow oil.

UPLC-MS (ES⁺, Method 2): 1.62 min, 687.6 [M+H]⁺.

Step B, 1-[4-(8-ethyl-7-fluoro-3-hydroxy-1-naphthyl)-2-methyl-7-[(2R,8S)-2-fluoro-1,2,3,5,6,7-hexahydropyrrolizin-8-yl]methoxy]pyrazolo[4,3-f]quinazolin-9-yl]-4-methyl-piperidin-4-ol. Trifluoroacetic acid (0.14 mL, 1.82 mmol) was added to 1-[4-[8-ethyl-7-fluoro-3-(methoxymethoxy)-1-naphthyl]-2-methyl-7-[(2R,8S)-2-fluoro-1,2,3,5,6,7-hexahydropyrrolizin-8-yl]methoxy]pyrazolo[4,3-f]quinazolin-9-yl]-4-methyl-piperidin-4-ol (50. mg, 0.07 mmol) and Triethylsilane (0.06 mL, 0.36 mmol) in DCM (0.6639 mL), and stirred at room temperature for 1 hour. The reaction mixture was passed through an SCX-2 column, washed with 2 CV MeOH, and eluted with 2 CV 1N NH₃/MeOH. The eluent was concentrated in vacuo and the residue purified by reverse phase chromatography (24 g, 10-35% MeCN in water, (0.1% formic acid)) desired fractions were combined and passed through an SCX-2 cartridge (5 g) eluting first with MeOH then 1 M NH₃ in MeOH. The ammoniacal fraction was concentrated under reduced pressure to afford 1-[4-(8-ethyl-7-fluoro-3-hydroxy-1-naphthyl)-2-methyl-7-[[(2R,8S)-2-fluoro-1,2,3,5,6,7-hexahydropyrrolizin-8-yl]methoxy]pyrazolo[4,3-f]quinazolin-9-yl]-4-methyl-piperidin-4-ol (25 mg, 0.0389 mmol, 53% yield) as a colourless solid.

UPLC-MS (ES⁺, Method 1): 2.94 min, 643.5 [M+H]⁺.

¹H NMR (400 MHz, DMSO-d6) δ/ppm: 9.79 (1H, s), 8.44 (1H, s), 7.74 (1H, dd, J=9.1, 6.1 Hz), 7.35-7.27 (3H, m), 6.97 (1H, d, J=2.5 Hz), 5.29 (1H, d, J=53.6 Hz), 4.44 (1H, s), 4.16-4.01 (5H, m), 3.65-3.41 (3H, m), 3.16-2.78 (4H, m), 2.42-2.35 (1H, m), 2.22-1.95 (5H, m), 1.90-1.73 (5H, m), 1.70-1.61 (2H, m), 1.28-1.22 (3H, m), 0.56-0.50 (3H, m).

The following examples in table 28 were made by analogy with example 118 (scheme 13) starting from the intermediate and building blocks for step A as described in the table.

TABLE 28

| | | UPLC- | ¹H NMR |
|---|---|---|---|
| | | MS | (400 MHz, |
| Step A Intermediate and | | (ES⁺): | DMSO-d₆) |
| building block | Example Structure/Name | min, m/z | δ/ppm |
| 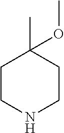 | 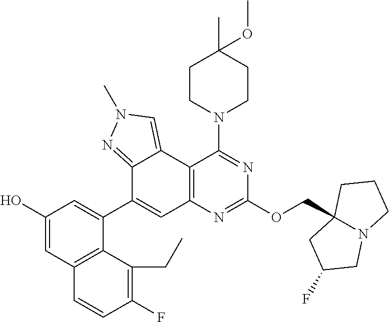 [SMILES: CCc1c(F)ccc2cc(O)cc(-c3cc4nc(OC[C@@]56CCCN5C[C@H](F)C6)nc(N5CCC(C)(OC)CC5)c4c4cn(C)nc34)c12] | 3.16 min, 657.8 [M + H]+ (Method 1) | 9.79 (1H, s), 8.46 (1H, s), 7.74 (1H, dd, J = 9.1, 6.3 Hz), 7.35 (1H, s), 7.31 (1H, t, J = 9.4 Hz), 7.28 (1H, d, J = 7.3 Hz), 6.97 (1H, d, J = 2.4 Hz), 5.40-5.20 (1H, m), 4.19-3.99 (5H, m), 3.66-3.55 |
| | | | (2H, m), |
| Intermediate I-138 and | Example 119 | | 3.19 (3H, |
| 4-methoxy-4-methyl- | 5-ethyl-6-fluoro-4-[9-(4-methoxy-4- | | s), 3.16- |
| piperidine | methyl-1-piperidyl)-2-methyl-7- | | 2.78 (4H |
| | [[(2R,8S)-2-fluoro-1,2,3,5,6,7- | | m), 2.41- |
| | hexahydropyrrolizin-8- | | 2.34 (1H, |
| | yl]methoxy]pyrazolo[4,3- | | m), 2.17- |
| | f]quinazolin-4-yl]naphthalen-2-ol | | 1.97 (5H, |
| | | | m), 1.92- |
| | | | 1.71 (8H, |
| | | | m), 1.34- |
| | | | 1.18 (3H, |
| | | | m), 0.56- |
| | | | 0.50 (3H, |
| | | | m). |
| 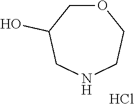 | 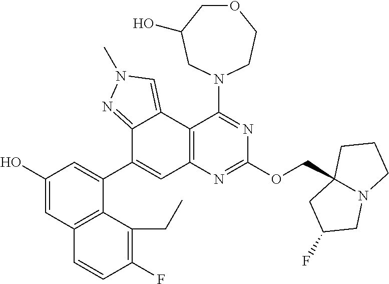 [SMILES: CCc1c(F)ccc2cc(O)cc(-c3cc4nc(OC[C@@]56CCCN5C[C@H](F)C6)nc(N5CCOCC(O)C5)c4c4cn(C)nc34)c12] | 2.80 min, 645.5 [M + H]+ (Method 1) | 9.79 (1H, s), 8.49 (1H, d, J = 6.7Hz), 7.74 (1H, dd, J = 9.0, 6.1Hz), 7.36-7.24 (3H, m), 6.99-6.96 (1H, m), 5.42-5.13 (2H, m), 4.21-3.96 (7H, m), 3.94-3.52 (7H, m), |
| | | | 3.23-2.96 |
| Intermediate I-138 and | Example 120 | | (3H, m), |
| [1,4]Oxazepan-6-ol | 4-[4-(8-ethyl-7-fluoro-3-hydroxy-1- | | 2.94-2.80 |
| | naphthyl)-2-methyl-7-[[(2R,8S)-2- | | (1H, m), |
| | fluoro-1,2,3,5,6,7- | | 2.42-2.32 |
| | hexahydropyrrolizin-8- | | (2H, m), |
| | yl]methoxy]pyrazolo[4,3- | | 2.28-1.70 |
| | f]quinazolin-9-yl]-1,4-oxazepan-6-ol | | (8H, m), |
| | | | 0.56-0.48 |
| | | | (3H, m). |
| 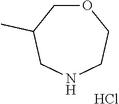 | 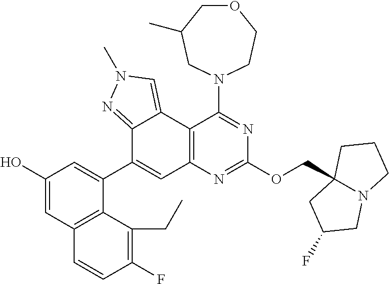 [SMILES: CCc1c(F)ccc2cc(O)cc(-c3cc4nc(OC[C@@]56CCCN5C[C@H](F)C6)nc(N5CCOCC(C)C5)c4c4cn(C)nc34)c12] | 2.95 min, 643.7 [M + H]+ (Method 1) | 9.79 (1H, s), 8.45 (1H, d, J = 10.2Hz), 7.74 (1H, dd, J = 9.0, 6.0Hz), 7.34-7.26 (3H, m), 6.98 (1H, d, J = 2.2Hz), 5.38-5.19 (1H, m), 4.15-4.00 (5H, m), 4.00-3.59 (6H, m), |
| | | | 3.54-3.35 |
| Intermediate I-138 and | Example 121 | | (2H, m), |
| 6-methyl-1,4-oxazepane | 5-ethyl-6-fluoro-4-[2-methyl-9-(6- | | 3.16-3.00 |
| | methyl-1,4-oxazepan-4-yl)-7- | | (3H, m), |
| | [[(2R,8S)-2-fluoro-1,2,3,5,6,7- | | 2.87-2.79 |
| | hexahydropyrrolizin-8- | | (1H, m), |
| | yl]methoxy]pyrazolo[4,3- | | 2.46-2.35 |
| | f]quinazolin-4-yl]naphthalen-2-ol | | (1H, m), |
| | | | 2.30-1.95 |
| | | | (5H, m), |
| | | | 1.90-1.71 |
| | | | (3H, m), |
| | | | 0.82-0.76 |
| | | | (3H, m), |
| | | | 0.54-0.48 |
| | | | (3H, m). |
| 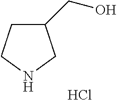 | 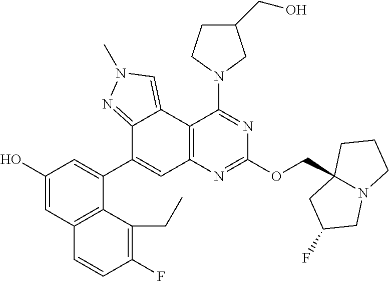 [SMILES: CCc1c(F)ccc2cc(O)cc(-c3cc4nc(OC[C@@]56CCCN5C[C@H](F)C6)nc(N5CCC(CO)C5)c4c4cn(C)nc34)c12] | 2.63 min, 629.5 [M + H]+ (Method 1) | 10.90 (br s, 1H), 9.89- 9.82 (m, 1H), 8.47 (s, 1H), 7.78-7.70 (m, 1H), 7.36-7.26 (m, 3H), 7.02-6.97 (m, 1H), 5.55 (d, J = 52.5 Hz, 1H), 4.81- 4.70 (m, 1H), 4.53 (br s, 2H), |
| | | | 4.11-4.03 |
| Intermediate I-138 and | Example 122 | | (m, 3H), |
| pyrrolidin-3- | 5-ethyl-6-fluoro-4-[9-[3- | | 3.95-3.36 |
| ylmethanol;hydrochloride | (hydroxymethyl)pyrrolidin-1-yl]-2- | | (m, 9H), |
| | methyl-7-[[(2R,8S)-2-fluoro- | | 3.30-3.15 |
| | 1,2,3,5,6,7-hexahydropyrrolizin-8- | | (m, 1H), |
| | yl]methoxy]pyrazolo[4,3- | | 2.63-2.54 |
| | f]quinazolin-4-yl]naphthalen-2-ol | | (m, 0.4H), |
| | | | 2.47-2.34 |
| | | | (m, 3H), |
| | | | 2.31-2.26 |
| | | | (m, 0.6H), |
| | | | 2.22-1.95 |
| | | | (m, 5H), |
| | | | 1.84-1.63 |
| | | | (m, 1H), |
| | | | 0.52 (t, J = |
| | | | 7.3Hz, 3H). |
| 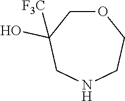 [SMILES: OC1(C(F)(F)F)CNCCOC1] | 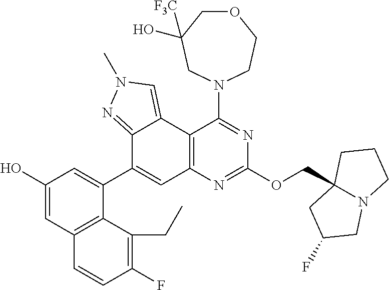 [SMILES: CCc1c(F)ccc2cc(O)cc(-c3cc4nc(OC[C@@]56CCCN5C[C@H](F)C6)nc(N5CCOCC(O)(C(F)(F)F)C5)c4c4cn(C)nc34)c12] | 3.23 min, 713.7 [M + H]+ (Method 1) | 9.84-9.80 (m, 1H), 8.56 (s, 1H), 7.78- 7.71 (m, 1H), 7.45- 7.05 (m, 4H), 6.99 (s, 1H), 5.35 (d, J = 54.0Hz, 1H), 4.35- 3.76 (m, 12H), 3.73- 3.54 (m, 2H), 3.26- 3.03 (m, 2H), 2.98- |
| | | | 2.83 (m, |
| Intermediate I-138 and | Example 123 | | 1H), 2.42- |
| 6-(trifluoromethyl)-1,4- | 4-[4-(8-ethyl-7-fluoro-3-hydroxy-1- | | 2.28 (m |
| oxazepan-6-ol | naphthyl)-2-methyl-7-[[(2R,8S)-2- | | 1H), 2.27- |
| | fluoro-1,2,3,5,6,7- | | 1.99 (m, |
| | hexahydropyrrolizin-8- | | 4H), 1.97- |
| | yl]methoxy]pyrazolo[4,3- | | 1.68 (m, |
| | f]quinazolin-9-yl]-6-(trifluoromethyl)- | | 3H), 0.52 |
| | 1,4-oxazepan-6-ol | | (t, J = |
| | | | 7.2Hz, 3H). |
| 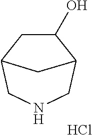 | 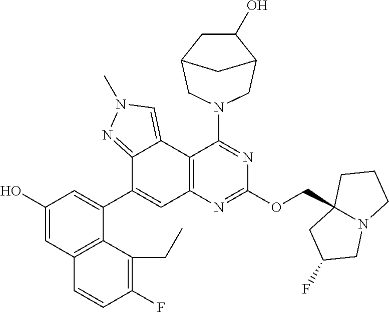 [SMILES: CCc1c(F)ccc2cc(O)cc(-c3cc4nc(OC[C@@]56CCCN5C[C@H](F)C6)nc(N5CC6CC(O)C(C6)C5)c4c4cn(C)nc34)c12] | 2.73 min, 655.5 [M + H]+ (Method 6) | 9.79 (s, 1H), 9.05- 8.78 (m, 1H), 7.79- 7.70 (m, 1H), 7.37- 7.26 (m, 3H), 7.00- 6.97 (m, 1H), 5.43- 5.35 (m, 0.5H), 5.29- 5.18 (1H), 4.99-4.90 (m, 0.5H), 4.29-4.10 (m, 3H), 4.10-3.99 (m, 5H), |
| | | | 3.93-3.82 |
| Intermediate I-138 and | Example 124 | | (m, 0.5H), |
| 3-azabicyclo[3.2.1]octan-6- | 3-[4-(8-ethyl-7-fluoro-3-hydroxy-1- | | 3.61-3.55 |
| ol hydrochloride | naphthyl)-2-methyl-7-[[(2R,8S)-2- | | (m, 0.5H), |
| | fluoro-1,2,3,5,6,7- | | 3.49-3.41 |
| | hexahydropyrrolizin-8- | | (m, 1H), |
| | yl]methoxy]pyrazolo[4,3- | | 3.23-3.00 |
| | f]quinazolin-9-yl]-3- | | (m, 3H), |
| | azabicyclo[3.2.1]octan-6-ol | | 2.95-2.82 |
| | | | (m, 1H), |
| | | | 2.42-2.35 |
| | | | (m, 1H), |
| | | | 2.32-2.25 |
| | | | (m, 1H), |
| | | | 2.25-2.15 |
| | | | (m, 1H), |
| | | | 2.14-1.98 |
| | | | (m, 5H), |
| | | | 1.94-1.76 |
| | | | (m, 3H), |
| | | | 1.75-1.67 |
| | | | (m, 2H), |
| | | | 1.54-1.39 |
| | | | (m, 1H), |
| | | | 0.58-0.48 |
| | | | (m, 3H). |
| 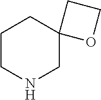 | 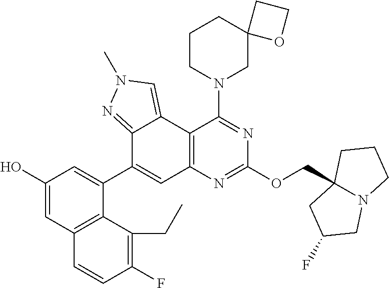 [SMILES: CCc1c(F)ccc2cc(O)cc(-c3cc4nc(OC[C@@]56CCCN5C[C@H](F)C6)nc(N5CCCC6(CCO6)C5)c4c4cn(C)nc34)c12] | 1.48 min, 655.5 [M + H]+ (Method 1) | 9.81 (s, 1H), 8.48- 8.35 (m, 1H), 7.75 (dd, J = 9.0, 6.1Hz, 1H), 7.38- 7.26 (m, 3H), 6.98 (d, J = 2.5Hz, 1H), 6.24-5.65 (m, 1H), 5.37 (d, J = 50.1Hz, 1H), 4.62- 4.49 (m, 1H), 4.39- |
| | | | 4.13 (m, |
| 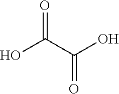 | Example 125 5-ethyl-6-fluoro-4-[2-methyl-9-(1- oxa-8-azaspiro[3.5]nonan-8-yl)-7- [[(2R,8S)-2-fluoro-1,2,3,5,6,7- hexahydropyrrolizin-8- yl]methoxy]pyrazolo[4,3- f]quinazolin-4-yl]naphthalen-2-ol | | 2H), 4.11- 4.01 (m, 3H), 3.87-3.69 (m, 2H), 3.58-3.47 (m, 2H), |
| | | | 3.05-2.84 |
| Intermediate I-138 and | | | (m, 2H), |
| 1-Oxa-6- | | | 2.41-2.34 |
| azaspiro[3.5]nonane | | | (m, 2H), |
| hemioxalate | | | 2.27-2.03 |
| | | | (m, 10H), |
| | | | 2.01-1.72 |
| | | | (m, 4H), |
| | | | 0.63-0.45 |
| | | | (m, 3H). |
| 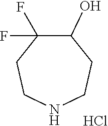 | 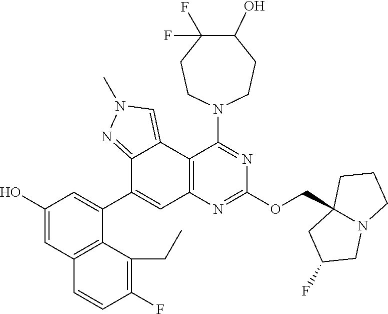 [SMILES: CCc1c(F)ccc2cc(O)cc(-c3cc4nc(OC[C@@]56CCCN5C[C@H](F)C6)nc(N5CCC(O)C(F)(F)CC5)c4c4cn(C)nc34)c12] | 2.88 min, 679.5 [M + H]+ (Method 1) | 9.82-9.78 (m, 1H), 8.58-8.51 (m, 1H), 7.78-7.72 (m, 1H), 7.36-7.26 (m, 3H), 7.00-6.95 (m, 1H), 5.77 (dd, J = 13.6, 5.8 Hz, 1H), 5.39-5.19 (m, 1H), 4.15-3.72 (m, 9H), 3.72-3.58 (m, 1H), |
| | | | 3.15-3.00 |
| Intermediate I-138 and | Example 126 | | (m, 3H), |
| 5,5-difluoroazepan-4-ol | 1-[4-(8-ethyl-7-fluoro-3-hydroxy-1- | | 2.88-2.79 |
| HCl | naphthyl)-2-methyl-7-[[(2R,8S)-2- | | (m, 1H), |
| | fluoro-1,2,3,5,6,7- | | 2.42-2.36 |
| | hexahydropyrrolizin-8- | | (m, 1H), |
| | yl]methoxy]pyrazolo[4,3- | | 2.31-2.22 |
| | f]quinazolin-9-yl]-5,5-difluoro- | | (m, 1H), |
| | azepan-4-ol | | 2.22-1.72 |
| | | | (m, 10H), |
| | | | 0.58-0.48 |
| | | | (m, 3H). |
| 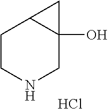 | 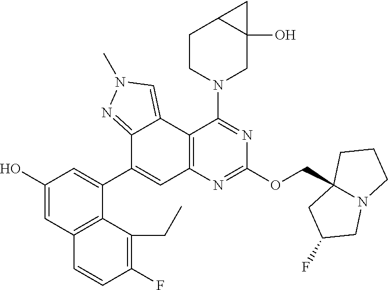 [SMILES: CCc1c(F)ccc2cc(O)cc(-c3cc4nc(OC[C@@]56CCCN5C[C@H](F)C6)nc(N5CCC6CC6(O)C5)c4c4cn(C)nc34)c12] | 2.91 min, 641.6 [M + H]+ (Method 1) | 9.80 (d, J = 2.0 Hz, 1H), 8.43 (d, J = 1.3 Hz, 1H), 7.74 (dd, J = 9.0, 6.1 Hz, 1H), 7.35-7.26 (m, 3H), 6.98 (d, J = 2.4 Hz, 1H), 5.64 (d, J = 11.3 Hz, 1H), 5.29 (d, J = 53.1 Hz, 1H), 4.17- |
| | | | 3.96 (m |
| Intermediate I-138 and | Example 127 | | 6H), 3.77- |
| 3-Azabicyclo[4.1.0]heptan- | 3-[4-(8-ethyl-7-fluoro-3-hydroxy-1- | | 3.69 (m, |
| 1-ol hydrochloride | naphthyl)-2-methyl-7-[[(2R,8S)-2- | | 1H), 3.64- |
| | fluoro-1,2,3,5,6,7- | | 3.49 (m, |
| | hexahydropyrrolizin-8- | | 1H), 3.25- |
| | yl]methoxy]pyrazolo[4,3- | | 2.98 (m, |
| | f]quinazolin-9-yl]-3- | | 4H), 2.88- |
| | azabicyclo[4.1.0]heptan-1-ol | | 2.79 (m, |
| | | | 1H), 2.44- |
| | | | 2.34 (m, |
| | | | 11H), 2.27- |
| | | | 1.93 (m, |
| | | | 6H), 1.91- |
| | | | 1.70 (m, |
| | | | 3H), 1.30- |
| | | | 1.21 (m, |
| | | | 1H), 0.82- |
| | | | 0.77 (m, |
| | | | 1H), 0.57- |
| | | | 0.50 (m, |
| | | | 3H), 0.42- |
| | | | 0.35 (m, |
| | | | 1H). |
| 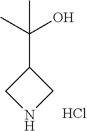 | 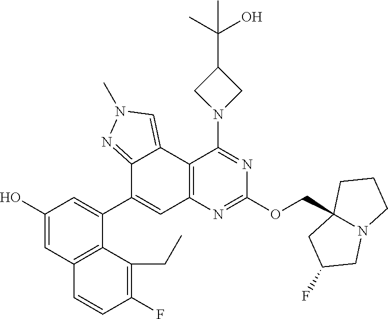 [SMILES: CCc1c(F)ccc2cc(O)cc(-c3cc4nc(OC[C@@]56CCCN5C[C@H](F)C6)nc(N5CC(C(C)(C)O)C5)c4c4cn(C)nc34)c12] | 2.84 min, 643.7 [M + H]+ (Method 1) | 9.79 (s, 1H), 8.46 (s, 1H), 7.74 (dd, J = 9.1, 6.1 Hz, 1H), 7.34-7.26 (m, 3H), 6.96 (d, J = 2.6 Hz, 1H), 5.29 (d, J = 54.5 Hz, 1H), 4.51 (s, 1H), 4.46- 4.38 (m, 1H), 4.32- 4.24 (m, 1H), 4.19- 4.06 (m, |
| | | | 5H), 4.05- |
| Intermediate I-138 and | Example 128 | | 3.98 (m, |
| 2-(azetidin-3-yl)propan-2-ol | 5-ethyl-6-fluoro-4-[9-[3-(1-hydroxy- | | 2H), 3.18- |
| hydrochloride | 1-methyl-ethyl)azetidin-1-yl]-2- | | 2.99 (m, |
| | methyl-7-[[(2R,8S)-2-fluoro- | | 3H), 2.88- |
| | 1,2,3,5,6,7-hexahydropyrrolizin-8- | | 2.71 (m, |
| | yl]methoxy]pyrazolo[4,3- | | 2H), 2.43- |
| | f]quinazolin-4-yl]naphthalen-2-ol | | 2.31 (m, |
| | | | 1H), 2.22- |
| | | | 1.98 (m, |
| | | | 4H), 1.92- |
| | | | 1.72 (m, |
| | | | 3H), 1.09 |
| | | | (s, 3H) |
| | | | 1.08 (s, |
| | | | 3H), 0.57- |
| | | | 0.50 (m, |
| | | | 3H). |
| 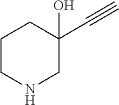 | 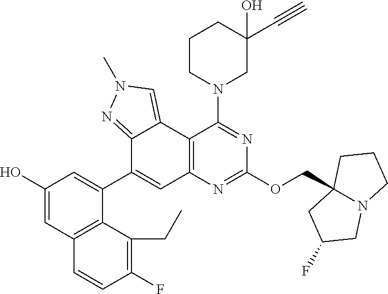 [SMILES: C#CC1(O)CCCN(c2nc(OC[C@@]34CCCN3C[C@H](F)C4)nc3cc(-c4cc(O)cc5ccc(F)c(CC)c45)c4nn(C)cc4c23)C1] | 2.97 min, 653.5 [M + H]+ (Method 1) | 9.81 (1H, d, J = 5.9 Hz), 8.54 (1H, d, J = 5.9 Hz), 7.75 (1H, dd, J = 9.0, 6.1 Hz), 7.38-7.26 (3H, m), 6.98 (1H, dd, J = 7.3, 2.9 Hz), 5.78-5.71 (1H, m) 5.50-5.20 (1H, m), 4.10-4.00 |
| | | | (4H, m), |
| Intermediate I-138 and | Example 129 | | 3.85-3.70 |
| 3-ethynylpiperidin-3-ol | 1-[4-(8-ethyl-7-fluoro-3-hydroxy-1- | | (2H, m), |
| | naphthyl)-2-methyl-7-[[(2R,8S)-2- | | 3.45-3.40 |
| | fluoro-1,2,3,5,6,7- | | (1H, m), |
| | hexahydropyrrolizin-8- | | 3.13-2.77 |
| | yl]methoxy]pyrazolo[4,3- | | (3H, m), |
| | f]quinazolin-9-yl]-3-ethynyl- | | 2.43-2.35 |
| | piperidin-3-ol | | (3H, m), |
| | | | 2.13-1.75 |
| | | | (10H, m), |
| | | | 1.72-1.60 |
| | | | (1H, m), |
| | | | 0.90-0.80 |
| | | | (1H, m), |
| | | | 0.60-0.50 |
| | | | (3H, m). |
| 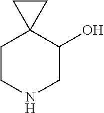 | 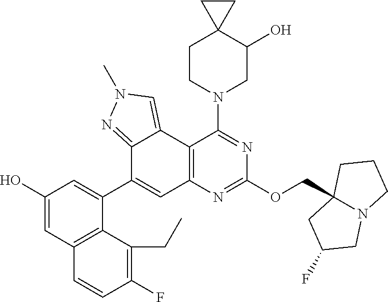 [SMILES: CCc1c(F)ccc2cc(O)cc(-c3cc4nc(OC[C@@]56CCCN5C[C@H](F)C6)nc(N5CCC6(CC6)C(O)C5)c4c4cn(C)nc34)c12] | 3.04 min, 655.7 [M + H]+ (Method 1) | 9.80 (1H, s), 8.72- 8.60 (1H m), 7.74 (1H, dd, J = 9.1, 6.1 Hz), 7.36- 7.26 (3H, m), 6.97 (1H, d, J = 2.5 Hz), 5.29 (1H, d, J = 54.8 Hz), 4.95 (1H, br. s), 4.16-4.01 (5H, m), 3.70-3.45 (4H, m), |
| | | | 3.16-3.00 |
| Intermediate I-138 and | Example 130 | | (3H, m), |
| 6-azaspiro[2.5]octan-8-ol | 6-[4-(8-ethyl-7-fluoro-3-hydroxy-1- | | 2.88-2.77 |
| | naphthyl)-2-methyl-7-[[(2R,8S)-2- | | (1H, m), |
| | fluoro-1,2,3,5,6,7- | | 2.41-2.34 |
| | hexahydropyrrolizin-8- | | (1H, m), |
| | yl]methoxy]pyrazolo[4,3- | | 2.23-1.72 |
| | f]quinazolin-9-yl]-6- | | (9H, m), |
| | azaspiro[2.5]octan-8-ol | | 1.70-1.55 |
| | | | (1H, br. s), |
| | | | 0.66-0.57 |
| | | | (2H, m), |
| | | | 0.57-0.50 |
| | | | (3H, m), |
| | | | 0.35-0.24 |
| | | | (2H, m). |
| 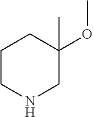 | 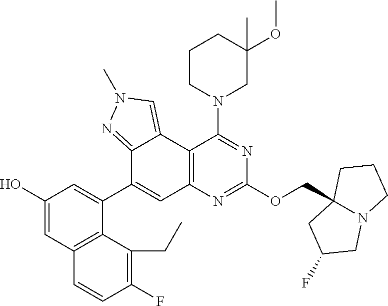 [SMILES: CCc1c(F)ccc2cc(O)cc(-c3cc4nc(OC[C@@]56CCCN5C[C@H](F)C6)nc(N5CCCC(C)(OC)C5)c4c4cn(C)nc34)c12] | 3.12 min, 657.6 [M + H]+ (Method 1) | 9.79 (1H, d, J = 5.7 Hz), 8.51 (1H, d, J = 13.4 Hz), 7.74 (1H, dd, J = 9.0, 6.1 Hz), 7.35-7.26 (3H, m), 7.00-6.97 (1H, m), 5.28 (1H, d, J = 54.9 Hz), 4.20- 4.01 (5H, m), 4.01- 3.54 (2H, m), 2.41- |
| | | | 2.34 |
| Intermediate I-138 and | Example 131 | | (1H, m), |
| 3-methoxy-3- | 5-ethyl-6-fluoro-4-[9-(3-methoxy-3- | | 2.26-1.53 |
| methylpiperidine | methyl-1-piperidyl)-2-methyl-7- | | (12H, m), |
| | [[(2R,8S)-2-fluoro-1,2,3,5,6,7- | | 1.05 (3H, |
| | hexahydropyrrolizin-8- | | s), 0.56- |
| | yl]methoxy]pyrazolo[4,3- | | 0.47 (3H, |
| | f]quinazolin-4-yl]naphthalen-2-ol | | m). |
| 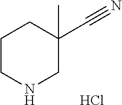 | 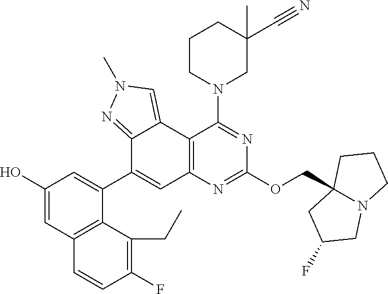 [SMILES: CCc1c(F)ccc2cc(O)cc(-c3cc4nc(OC[C@@]56CCCN5C[C@H](F)C6)nc(N5CCCC(C)(C#N)C5)c4c4cn(C)nc34)c12] | 3.18 min, 652.7 [M + H]+ (Method 1) | 9.81 (1H, d, J = 4.3 Hz), 8.41 (1H, d, J = 3.4 Hz), 7.75 (1H, dd, J = 9.1, 6.1 HZ), 7.42-7.27 (3H, m), 7.00-6.96 (1H, m), 5.29 (1H, d, J = 53.2 Hz), 4.25- 3.72 (7H, m), 3.26- 2.97 (4H, |
| | | | m), 2.93- |
| Intermediate I-138 and | Example 132 | | 2.71 (2H, |
| 3-methylpiperidine-3- | 1-[4-(8-ethyl-7-fluoro-3-hydroxy-1- | | m), 2.43- |
| carbonitrile hydrochloride | naphthyl)-2-methyl-7-[[(2R,8S)-2- | | 2.34 (1H, |
| | fluoro-1,2,3,5,6,7- | | m), 2.30- |
| | hexahydropyrrolizin-8- | | 1.61 (11H, |
| | yl]methoxy]pyrazolo[4,3- | | m), 1.43 |
| | f]quinazolin-9-yl]-3-methyl- | | (3H, s), |
| | piperidine-3-carbonitrile | | 0.64-0.44 |
| | | | (3H, m). |
| 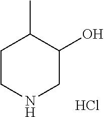 | 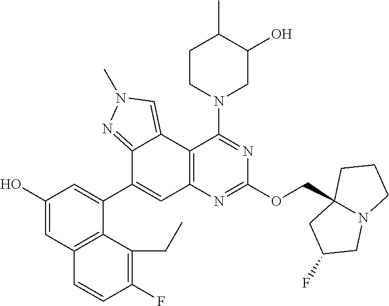 [SMILES: CCc1c(F)ccc2cc(O)cc(-c3cc4nc(OC[C@@]56CCCN5C[C@H](F)C6)nc(N5CCC(C)C(O)C5)c4c4cn(C)nc34)c12] | 3.02 min, 643.5 [M + H]+ (Method 1) | 9.79 (1H, d, J = 1.4 Hz), 8.83- 8.77 (1H, m), 7.74 (1H, dd, J = 9.0, 6.1 Hz), 7.36- 7.26 (3H, m), 6.97 (1H, d, J = 2.5 Hz), 5.29 (1H, d, J = 54.9 Hz), 4.91- 4.79 (1H, m), 4.20- 3.97 (5H, m), 3.90- |
| | | | 3.71 (3H, |
| Intermediate I-138 and | Example 133 | | m), 3.20- |
| 4-methylpiperidin-3-ol | 1-[4-(8-ethyl-7-fluoro-3-hydroxy-1- | | 2.79 (5H, |
| hydrochloride | naphthyl)-2-methyl-7-[[(2R,8S)-2- | | m), 2.22- |
| | fluoro-1,2,3,5,6,7- | | 2.00 (5H, |
| | hexahydropyrrolizin-8- | | m), 1.93- |
| | yl]methoxy]pyrazolo[4,3- | | 1.74 (6H, |
| | f]quinazolin-9-yl]-4-methyl- | | m), 1.62- |
| | piperidin-3-ol | | 1.54 (1H, |
| | | | m), 1.01 |
| | | | (3H, d, J = |
| | | | 6.0 Hz), |
| | | | 0.59-0.48 |
| | | | (3H, m). |
| 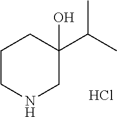 | 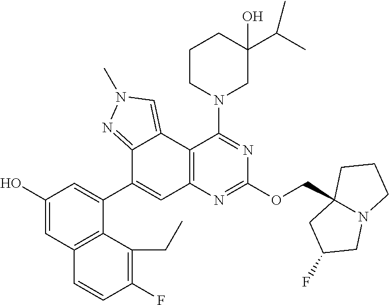 [SMILES: CCc1c(F)ccc2cc(O)cc(-c3cc4nc(OC[C@@]56CCCN5C[C@H](F)C6)nc(N5CCCC(O)(C(C)C)C5)c4c4cn(C)nc34)c12] | 3.18 min, 671.7 [M + H]+ (Method 1) | 9.79 (1H, d, J = 3.1 Hz), 8.75 (1H, br. s), 7.74 (1H, dd, J = 9.0, 6.1 Hz), 7.35-7.26 (3H, m), 6.98 (1H, d, J = 2.6 Hz), 5.29 (1H, d, J = 53.9 Hz), 4.56-4.29 (1H, m), 4.21-3.99 (5H, m), 3.95-3.55 |
| | | | (2H, m), |
| Intermediate I-138 and | Example 134 | | 3.18-2.74 |
| 3-(propan-2-yl)piperidin-3- | 1-[4-(8-ethyl-7-fluoro-3-hydroxy-1- | | (5H, m), |
| ol hydrochloride | naphthyl)-2-methyl-7-[[(2R,8S)-2- | | 2.42-2.34 |
| | fluoro-1,2,3,5,6,7- | | (1H, m), |
| | hexahydropyrrolizin-8- | | 2.23 (5H, |
| | yl]methoxy]pyrazolo[4,3- | | m), 1.94- |
| | f]quinazolin-9-yl]-3-isopropyl- | | 1.41 (8H, |
| | piperidin-3-ol | | m), 0.86 |
| | | | (3H, t, J = |
| | | | 6.1 Hz), |
| | | | 0.80-0.57 |
| | | | (3H, m), |
| | | | 0.55-0.45 |
| | | | (3H, m). |
| 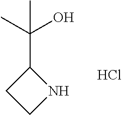 | 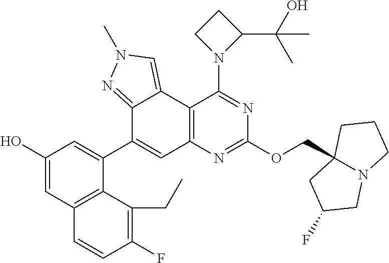 [SMILES: CCc1c(F)ccc2cc(O)cc(-c3cc4nc(OC[C@@]56CCCN5C[C@H](F)C6)nc(N5CCC5C(C)(C)O)c4c4cn(C)nc34)c12] | 2.84 min, 643.7 [M + H]+ (Method 1) | 9.79 (s, 1H), 8.46 (s, 1H), 7.74 (dd, J = 9.1, 6.1 Hz, 1H), 7.34-7.26 (m, 3H), 6.96 (d, J = 2.6 Hz 1H), 5.29 (d, J = 54.5 Hz, 1H), 4.51 (s, 1H), 4.46- 4.38 (m, |
| | | | 1H), 4.32- |
| Intermediate I-138 and | Example 135 | | 4.24 (m, |
| 2-(azetidin-2-yl)propan-2-ol | 5-ethyl-6-fluoro-4-[9-[2-(1-hydroxy- | | 1H), 4.19- |
| hydrochloride | 1-methyl-ethyl)azetidin-1-yl]-2- | | 4.06 (m, |
| | methyl-7-[[(2R,8S)-2-fluoro- | | 5H), 4.05- |
| | 1,2,3,5,6,7-hexahydropyrrolizin-8- | | 3.98 (m, |
| | yl]methoxy]pyrazolo[4,3- | | 2H), 3.18- |
| | f]quinazolin-4-yl]naphthalen-2-ol | | 2.99 (m, |
| | | | 3H), 2.88- |
| | | | 2.71 (m, |
| | | | 2H), 2.43- |
| | | | 2.31 (m, |
| | | | 1H), 2.22- |
| | | | 1.98 (m, |
| | | | 4H), 1.92- |
| | | | 1.72 (m, |
| | | | 3H), 1.09 |
| | | | (s, 3H), |
| | | | 1.08 (s, |
| | | | 3H), 0.57- |
| | | | 0.50 (m, |
| | | | 3H). |
| 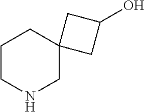 | 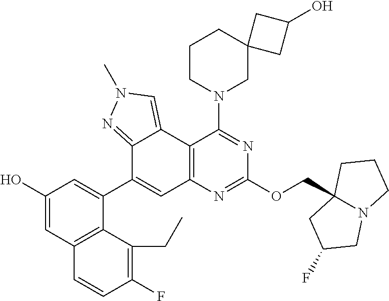 [SMILES: CCc1c(F)ccc2cc(O)cc(-c3cc4nc(OC[C@@]56CCCN5C[C@H](F)C6)nc(N5CCCC6(CC(O)C6)C5)c4c4cn(C)nc34)c12] | 2.90 min, 669.8 [M + H]+ (Method 1) | 9.79 (1H, s), 8.31 (1H, d, J = 3.5 Hz), 7.74 (1H, dd, J = 9.1, 6.0 Hz), 7.36-7.27 (3H, m), 6.99 (1H, d, J = 2.4 Hz), 5.29 (1H, d, J = 54.3 Hz), 4.85 (1H, dd, J = 40.2, 6.3 Hz), 4.17- 3.82 (6H, |
| | | | m), 3.53- |
| Intermediate I-138 and | Example 136 | | 3.36 (2H, |
| 6-azaspiro[3.5]nonan-2-ol | 6-[4-(8-ethyl-7-fluoro-3-hydroxy-1- | | m), 3.16- |
| | naphthyl)-2-methyl-7-[[(2R,8S)-2- | | 3.00 (3H, |
| | fluoro-1,2,3,5,6,7- | | m), 2.88- |
| | hexahydropyrrolizin-8- | | 2.79 (1H, |
| | yl]methoxy]pyrazolo[4,3- | | m), 2.43- |
| | f]quinazolin-9-yl]-6- | | 2.34 (1H, |
| | azaspiro[3.5]nonan-2-ol | | m), 2.21- |
| | | | 1.30 (17H, |
| | | | m), 0.57- |
| | | | 0.49 (3H, |
| | | | m). |
| 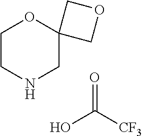 | 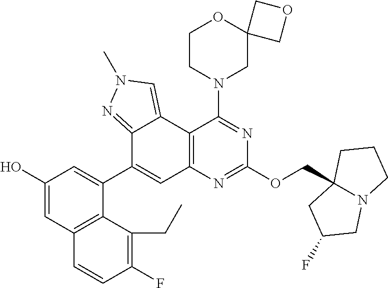 [SMILES: CCc1c(F)ccc2cc(O)cc(-c3cc4nc(OC[C@@]56CCCN5C[C@H](F)C6)nc(N5CCOC6(COC6)C5)c4c4cn(C)nc34)c12] | 3.03 min, 657.5 [M + H]+ (Method 1) | 9.82 (1H, s), 8.34 (1H, s), 7.75 (1H, dd, J = 9.1, 6.0 Hz), 7.41 (1H, s), 7.35- 7.28 (2H, m), 6.97 (1H, d, J = 2.3 Hz), 5.56-5.26 (1H, m), 4.61-4.44 (4H, m), 4.44-4.01 (5H, m), |
| | | | 4.00-3.89 |
| Intermediate I-138 and | Example 199 | | (2H, m), |
| 2,5-dioxa-8- | 4-[9-(2,5-dioxa-8- | | 3.86-3.74 |
| azaspiro[3.5]nonane;2,2,2- | azaspiro[3.5]nonan-8-yl)-2-methyl- | | (2H, m), |
| trifluoroacetic acid | 7-[[(2R,8S)-2-fluoro-1,2,3,5,6,7- | | 3.52-3.36 |
| | hexahydropyrrolizin-8- | | (2H, m), |
| | yl]methoxy]pyrazolo[4,3- | | 2.43-2.35 |
| | f]quinazolin-4-yl]-5-ethyl-6-fluoro- | | (2H, m), |
| | naphthalen-2-ol | | 2.26-1.72 |
| | | | (8H, m), |
| | | | 1.72-1.35 |
| | | | (2H, m), |
| | | | 0.57-0.51 |
| | | | (3H, m). |
| 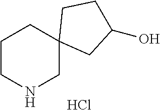 [SMILES: OC1CCC2(CCCNC2)C1 ; Cl] | 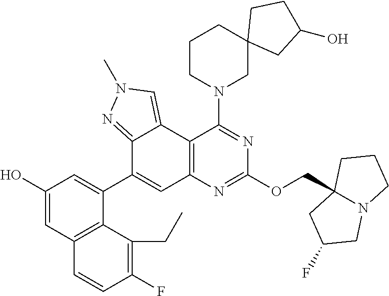 [SMILES: CCc1c(F)ccc2cc(O)cc(-c3cc4nc(OC[C@@]56CCCN5C[C@H](F)C6)nc(N5CCCC6(CCC(O)C6)C5)c4c4cn(C)nc34)c12] | 2.92 min, 683.5 [M + H]+ (Method 1) | 9.82 (1H, m), 8.34- 8.30 (1H, m), 7.77- 7.69 (1H, m), 7.37-7.24 (3H, m), 6.99-6.96 (1H, m), 5.37-5.19 (1H, m), 4.50-4.30 (1H, m), 4.16-3.95 (6H, m), 3.66-3.50 (1H, m), |
| | | | 3.25-3.00 |
| Intermediate I-138 and | Example 200 | | (5H, m), |
| 7-azaspiro[4.5]decan-2-ol | 7-[4-(8-ethyl-7-fluoro-3-hydroxy-1- | | 2.88-2.79 |
| hydrochloride | naphthyl)-2-methyl-7-[[(2R,8S)-2- | | (1H, m), |
| | fluoro-1,2,3,5,6,7- | | 2.41-2.34 |
| | hexahydropyrrolizin-8- | | (1H, m), |
| | yl]methoxy]pyrazolo[4,3- | | 2.18-1.98 |
| | f]quinazolin-9-yl]-7- | | (5H, m), |
| | azaspiro[4.5]decan-3-ol | | 1.87-1.32 |
| | | | (13H, m), |
| | | | 0.56-0.48 |
| | | | (3H, m). |
| 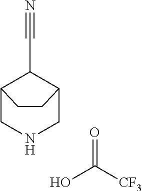 | 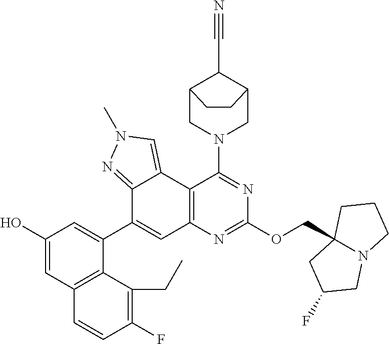 [SMILES: CCc1c(F)ccc2cc(O)cc(-c3cc4nc(OC[C@@]56CCCN5C[C@H](F)C6)nc(N5CC6CCC(C5)C6C#N)c4c4cn(C)nc34)c12] | 3.08 min, 664.5 [M + H]+ (Method 1 | 9.79 (s, 1H), 8.43- 8.35 (m, 1H), 7.74 (dd, J = 8.9, 6.3 Hz, 1H), 7.34- 7.26 (m, 3H), 6.99- 6.96 (m, 1H), 5.37- 5.19 (m, 1H), 4.28- 3.90 (m, 8H), 3.72- 3.40 (m, 3H), 3.16- 2.99 (m, 4H), 2.87- 2.78 (m, 1H), 2.21- |
| | | | 1.96 (m, |
| Intermediate I-138 and | Example 210 | | 5H), 1.88- |
| 3- | 3-[4-(8-ethyl-7-fluoro-3-hydroxy-1- | | 1.73 (m, |
| azoniabicyclo[3.2.1]octane- | naphthyl)-7-[[(2R,8S)-2-fluoro- | | 5H), 1.70- |
| 8-carbonitrile;2,2,2- | 1,2,3,5,6,7-hexahydropyrrolizin-8- | | 1.57 (m, |
| trifluoroacetate | yl]methoxy]-2-methyl-pyrazolo[4,3- | | 2H), 0.54 |
| | f]quinazolin-9-yl]-3- | | (t, J = 7.6 |
| | azabicyclo[3.2.1]octane-8- | | Hz, 3H) |
| | carbonitrile | | |

### Example 183, 5-ethyl-6-fluoro-4-[9-[2-(hydroxymethyl) pyrrolidin-1-yl]-2-methyl-7-[[(2R,8S)-2-fluoro-1,2,3,5,6,7-hexahydropyrrolizin-8-yl]methoxy]pyrazolo[4,3-f]quinazolin-4-yl]naphthalen-2-ol

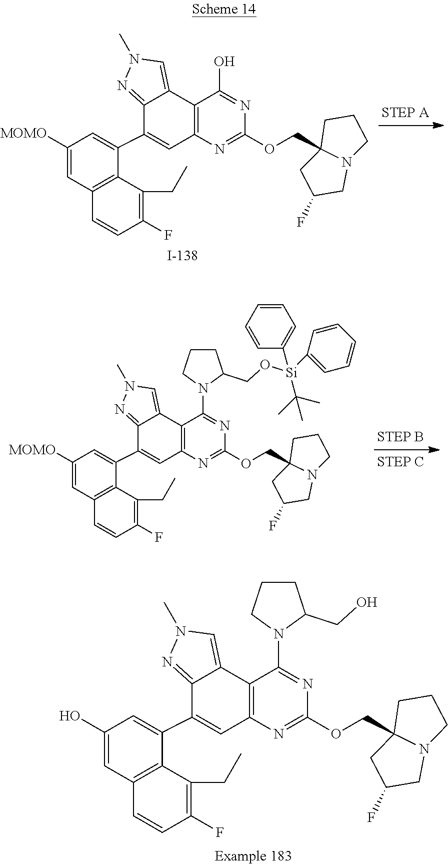

Step A, tert-butyl-[[1-[4-[8-ethyl-7-fluoro-3-(methoxymethoxy)-1-naphthyl]-2-methyl-7-[[(2R,8S)-2-fluoro-1,2,3,5,6,7-hexahydropyrrolizin-8-yl]methoxy]pyrazolo[4,3-f]quinazolin-9-yl]pyrrolidin-2-yl]methoxy]-diphenyl-silane. To a solution of 4-[8-ethyl-7-fluoro-3-(methoxymethoxy)-1-naphthyl]-2-methyl-7-[(2R,8S)-2-fluoro-1,2,3,5,6,7-hexahydropyrrolizin-8-yl]methoxy]pyrazolo[4,3-f]quinazolin-9-ol (54. mg, 0.09 mmol) and N,N-Diisopropylethylamine (39.88 μL, 0.23 mmol) in DCM (1 mL) at 0° C., under N₂, were added 2-Nitrobenzolsulfonylchloride (24.35 mg, 0.11 mmol) and 4-dimethylamino pyridine (0.56 mg, 0. mmol). The mixture was stirred for 15 minutes the left to warm to room temperature and stirred for 2 h. The reaction mixture was diluted with DCM and water was added. The layers were separated and the organic layer was passed through a phase separator and concentrated in vacuo. The residue was taken up in DMA (0.7 mL) and tert-butyl-diphenyl-(pyrrolidin-2-ylmethoxy) silane (66. mg, 0.16 mmol) added. The mixture was cooled to 0° C. and potassium phosphate tribasic (116.64 mg, 0.55 mmol) was added. The reaction mixture was allowed to warm to room temperature and left to stir overnight. Water was added and the mixture was extracted with EtOAc (×3). The combined organic layers were washed with brine (2×10 mL), passed through a phase separator and concentrated to afford tert-butyl-[1-[4-[8-ethyl-7-fluoro-3-(methoxymethoxy)-1-naphthyl]-2-methyl-7-[rac-(2R,8S)-2-fluoro-1,2,3,5,6,7-hexahydropyrrolizin-8-yl]methoxy]pyrazolo[4,3-f]quinazolin-9-yl]pyrrolidin-2-yl]methoxy]-diphenyl-silane (83.45 mg, 0.0916 mmol, 100% yield) as a brown oil.

UPLC-MS (ES⁺, Method 2): 2.18 min, 456.6 [M+H]⁺.

Step B, 4-[9-[2-[[tert-butyl(diphenyl) silyl]oxymethyl]pyrrolidin-1-yl]-2-methyl-7-[[(2R,8S)-2-fluoro-1,2,3,5,6,7-hexahydropyrrolizin-8-yl]methoxy]pyrazolo[4,3-f]quinazolin-4-yl]-5-ethyl-6-fluoro-naphthalen-2-ol. 4N Hydrogen Chloride in dioxane (0.25 mL, 1. mmol) was added to a solution of tert-butyl-[[1-[4-[8-ethyl-7-fluoro-3-(methoxymethoxy)-1-naphthyl]-2-methyl-7-[(2R,8S)-2-fluoro-1,2,3,5,6,7-hexahydropyrrolizin-8-yl]methoxy]pyrazolo[4,3-f]quinazolin-9-yl]pyrrolidin-2-yl]methoxy]-diphenyl-silane (83.45 mg, 0.09 mmol) in DCM (1 mL). The mixture was stirred at room temperature 1 hour. The reaction mixture was concentrated to dryness, taken up in EtOAc and Water. NaHCO₃ (aq.sat.) was added and the layers separated. The aqueous was further extracted with EtOAc (×2). The combined organics were filtered through a phase separator and concentrated to afford 4-[9-[2-[tert-butyl(diphenyl) silyl]oxymethyl]pyrrolidin-1-yl]-2-methyl-7-[(2R,8S)-2-fluoro-1,2,3,5,6,7-hexahydropyrrolizin-8-yl]methoxy]pyrazolo[4,3-f]quinazolin-4-yl]-5-ethyl-6-fluoro-naphthalen-2-ol (79.4 mg, 0.0916 mmol, 100% yield) as an orange oil.

UPLC-MS (ES⁺, Method 2): 2.29 min, 434.0 (half mass) [M+H]⁺.

Step C, 5-ethyl-6-fluoro-4-[9-[2-(hydroxymethyl) pyrrolidin-1-yl]-2-methyl-7-[[(2R,8S)-2-fluoro-1,2,3,5,6,7-hexahydropyrrolizin-8-yl]methoxy]pyrazolo[4,3-f]quinazolin-4-yl]naphthalen-2-ol. To a solution of 4-[9-[2-[tert-butyl(diphenyl) silyl]oxymethyl]pyrrolidin-1-yl]-2-methyl-7-[(2R,8S)-2-fluoro-1,2,3,5,6,7-hexahydropyrrolizin-8-yl]methoxy]pyrazolo[4,3-f]quinazolin-4-yl]-5-ethyl-6-fluoro-naphthalen-2-ol (79 mg, 0.09 mmol) in THF (1 mL) was added Tetrabutylammonium fluoride 1.0M in THF (0.18 mL, 0.18 mmol). The reaction mixture was left to stir at room temperature overnight. The solvents were removed in vacuo and the crude purified by flash chromatography (5 g KP-NH, eluting in 0-6% MeOH in DCM) followed by reverse phase chromatography (12 g, eluting 10-50% MeCN in water (0.1% formic acid)). Product containing fractions were loaded onto a pre-equilibrated SCX-2 column (1 g, washing with MeOH and eluting with 1M NH₃/MeOH) to afford 5-ethyl-6-fluoro-4-[9-[2-(hydroxymethyl) pyrrolidin-1-yl]-2-methyl-7-[(2R,8S)-2-fluoro-1,2,3,5,6,7-hexahydropyrrolizin-8-yl]methoxy]pyrazolo[4,3-f]quinazolin-4-yl]naphthalen-2-ol (7 mg, 0.0111 mmol, 12% yield) as a white solid.

UPLC-MS (ES⁺, Method 1): 2.63 min, 629.5 [M+H]⁺.

¹H NMR (400 MHz, DMSO-d6)/ppm: 9.83-9.75 (m, 1H), 8.42-8.34 (m, 1H), 7.78-7.69 (m, 1H), 7.37-7.25 (m, 3H), 7.00-6.92 (m, 1H), 5.31 (d, J=54.6 Hz, 1H), 4.85-4.70 (m, 2H), 4.21-3.86 (m, 6H), 3.70-3.61 (m, 1H), 3.59-3.46 (m, 1H), 3.22-2.95 (m, 3H), 2.93-2.80 (m, 1H), 2.43-2.35 (m, 1H), 2.24-1.76 (m, 10H), 1.71-1.57 (m, 1H), 1.40-1.28 (m, 1H), 0.63-0.45 (m, 3H).

### Biological Results

### HTRF Nucleotide Exchange Assay Method

The capacity of compounds to bind KRAS G12D, other KRAS mutants and wildtype RAS isoforms was quantified using a HTRF nucleotide exchange assay. Recombinant human RAS protein (2 nM; aa1-188 KRAS WT, HRAS WT, NRAS WT, or KRAS containing the G12D, G13D or Q61H amino acid substitutions, or 4 nM KRAS; aa1-188 containing the G12V, G12C, G12A or G12S amino acid substitution, an N-terminal 6× His-tag and leader sequence), and 2 nM Europium-labeled anti-6× His antibody were mixed in assay buffer (10 mM HEPES pH 7.3, 150 mM NaCl, 5 mM MgCl₂, 0.05% BSA, 0.0025% NP-40 and 100 mM KF) with various concentrations of compound in a 384-well plate and a volume of 5 μL. After a 60-minute incubation at room temperature, 5 μl of 200 nM EDA-GTP-DY647P1 (diluted in assay buffer) was added to the plate. Following 30-minute incubation at room temperature, time-resolved fluorescence was measured on a PerkinElmer Envision plate reader. DMSO (0.3%) and unlabeled GDP (1 μM) or equivalent tool compound were used to generate the Max and Min assay signals, respectively. Data was analysed using a four-parameter logistic model to calculate IC₅₀ values, with at least two independent replicates were performed for each compound. The results are presented as IC₅₀s in Table 29 where “A” corresponds to an IC₅₀ ≤10 nM, “B” to an IC₅₀ >10 nM up to 100 nM, “C” is >100 nM up to 10 μM, “D” represents <49% inhibition at 10 μM and ND=not determined:

TABLE 29

| | KRAS^{G12D} | KRAS^{WT} | KRAS^{G12V} | KRAS^{G12S} | KRAS^{G12A} |
|---|---|---|---|---|---|
| Example | NEA | NEA | NEA | NEA | NEA |
| 1 | A | A | B | B | A |
| 2 | A | A | ND | ND | ND |
| 3 | D | C | ND | ND | ND |
| 4 | D | D | ND | ND | ND |
| 5 | C | D | ND | ND | ND |
| 6 | A | A | ND | ND | ND |
| 7 | A | A | A | ND | ND |
| 8 | A | A | ND | ND | ND |
| 9 | C | C | ND | ND | ND |
| 10 | A | A | ND | ND | ND |
| 11 | A | A | ND | ND | ND |
| 12 | B | B | ND | ND | ND |
| 13 | B | C | ND | ND | ND |
| 14 | A | C | B | ND | ND |
| 15 | A | B | ND | ND | ND |
| 16 | D | D | ND | ND | ND |
| 17 | A | A | ND | ND | ND |
| 18 | B | B | ND | ND | ND |
| 19 | C | C | ND | ND | ND |
| 20 | A | B | ND | ND | ND |
| 21 | A | A | ND | ND | ND |
| 22 | A | A | ND | ND | ND |
| 23 | A | A | ND | ND | ND |
| 24 | A | A | ND | ND | ND |
| 25 | A | C | ND | ND | ND |
| 26 | A | A | ND | ND | ND |
| 27 | C | C | ND | ND | ND |
| 28 | A | A | ND | ND | ND |
| 29 | D | C | ND | ND | ND |
| 30 | B | B | ND | ND | ND |
| 31 | C | C | ND | ND | ND |
| 32 | C | C | ND | ND | ND |
| 33 | B | B | ND | ND | ND |
| 34 | A | C | ND | ND | ND |
| 35 | C | D | ND | ND | ND |
| 36 | A | B | ND | ND | ND |
| 37 | C | D | ND | ND | ND |
| 38 | C | C | ND | ND | ND |
| 39 | B | B | ND | ND | ND |
| 40 | A | A | ND | ND | ND |
| 41 | B | B | ND | ND | ND |
| 42 | B | B | ND | ND | ND |
| 43 | A | A | ND | ND | ND |
| 44 | B | B | ND | ND | ND |
| 45 | C | C | ND | ND | ND |
| 46 | D | C | ND | ND | ND |
| 47 | C | C | ND | ND | ND |
| 48 | D | D | ND | ND | ND |
| 49 | D | C | ND | ND | ND |
| 50 | A | B | ND | ND | ND |
| 51 | A | B | ND | ND | ND |
| 52 | A | B | ND | ND | ND |
| 53 | C | D | ND | ND | ND |
| 54 | C | C | ND | ND | ND |
| 58 | D | D | ND | ND | ND |
| 59 | A | B | ND | ND | ND |
| 60 | B | C | ND | ND | ND |
| 61 | B | C | ND | ND | ND |
| 62 | B | C | ND | ND | ND |
| 63 | A | B | ND | ND | ND |
| 64 | B | C | ND | ND | ND |
| 65 | A | A | ND | ND | ND |
| 66 | B | B | ND | ND | ND |
| 68 | A | A | ND | ND | ND |
| 69 | A | B | ND | ND | ND |
| 70 | A | B | ND | ND | ND |
| 71 | D | D | ND | ND | ND |
| 72 | A | B | ND | ND | ND |
| 73 | A | B | ND | ND | ND |
| 74 | A | B | ND | ND | ND |
| 76 | A | B | ND | ND | ND |
| 77 | C | C | ND | ND | ND |
| 78 | A | C | ND | ND | ND |
| 79 | A | A | ND | ND | ND |
| 80 | A | A | ND | ND | ND |
| 81 | A | B | ND | ND | ND |
| 82 | A | A | ND | ND | ND |
| 83 | D | D | ND | ND | ND |
| 84 | A | B | ND | ND | ND |
| 86 | A | D | NC | ND | ND |
| 87 | A | A | ND | ND | ND |
| 88 | A | A | ND | ND | ND |
| 89 | A | C | ND | ND | ND |
| 90 | D | D | ND | ND | ND |
| 91 | A | C | ND | ND | ND |
| 92 | A | A | ND | ND | ND |
| 93 | A | A | ND | ND | ND |
| 94 | A | A | ND | ND | ND |
| 95 | A | A | ND | ND | ND |
| 96 | A | A | ND | ND | ND |
| 97 | A | A | ND | ND | ND |
| 98 | A | A | ND | ND | ND |
| 99 | A | A | ND | ND | ND |
| 100 | A | A | ND | ND | ND |
| 101 | A | A | ND | ND | ND |
| 102 | A | B | ND | ND | ND |
| 103 | A | A | ND | ND | ND |
| 104 | B | C | ND | ND | ND |
| 105 | A | A | A | B | A |
| 106 | D | D | ND | ND | ND |
| 107 | D | D | ND | ND | ND |
| 108 | C | C | ND | ND | ND |
| 109 | A | A | ND | ND | ND |
| 110 | A | A | ND | ND | ND |
| 111 | B | A | ND | ND | ND |
| 112 | A | A | ND | ND | ND |
| 113 | C | B | ND | ND | ND |
| 114 | A | B | ND | ND | ND |
| 115 | A | A | ND | ND | ND |
| 116 | B | B | ND | ND | ND |
| 117 | A | A | ND | ND | ND |
| 118 | C | C | ND | ND | ND |
| 119 | C | C | ND | ND | ND |
| 120 | A | A | ND | ND | ND |
| 121 | A | A | ND | ND | ND |
| 122 | A | A | ND | ND | ND |
| 123 | B | B | ND | ND | ND |
| 124 | A | A | ND | ND | ND |
| 125 | B | B | ND | ND | ND |
| 126 | B | B | ND | ND | ND |
| 127 | B | B | ND | ND | ND |
| 128 | C | D | ND | ND | ND |
| 129 | B | B | ND | ND | ND |
| 130 | D | C | ND | ND | ND |
| 131 | B | C | ND | ND | ND |
| 132 | C | C | ND | ND | ND |
| 133 | B | C | ND | ND | ND |
| 134 | C | D | ND | ND | ND |
| 135 | C | B | ND | ND | ND |
| 136 | A | A | ND | ND | ND |
| 137 | A | A | ND | ND | ND |
| 138 | A | A | ND | ND | ND |
| 139 | A | A | ND | ND | ND |
| 140 | A | A | ND | ND | ND |
| 141 | A | A | ND | ND | ND |
| 142 | A | A | ND | ND | ND |
| 143 | A | A | ND | ND | ND |
| 144 | A | B | ND | ND | ND |
| 145 | A | A | ND | ND | ND |
| 146 | A | A | ND | ND | ND |
| 147 | A | A | ND | ND | ND |
| 148 | A | A | ND | ND | ND |
| 149 | A | A | ND | ND | ND |
| 150 | A | A | ND | ND | ND |
| 151 | A | A | ND | ND | ND |
| 152 | A | A | ND | ND | ND |
| 153 | A | A | ND | ND | ND |
| 154 | A | A | ND | ND | ND |
| 155 | A | A | ND | ND | ND |
| 156 | A | A | ND | ND | ND |
| 157 | A | A | ND | ND | ND |
| 158 | A | A | ND | ND | ND |
| 159 | A | A | ND | ND | ND |
| 160 | A | A | ND | ND | ND |
| 161 | A | A | ND | ND | ND |
| 162 | A | A | ND | ND | ND |
| 163 | A | A | ND | ND | ND |
| 164 | A | B | ND | ND | ND |
| 165 | A | B | ND | ND | ND |
| 166 | A | A | ND | ND | ND |
| 167 | A | C | ND | ND | ND |
| 168 | A | C | ND | ND | ND |
| 169 | A | C | ND | ND | ND |
| 170 | A | A | ND | ND | ND |
| 171 | A | A | ND | ND | ND |
| 172 | A | A | ND | ND | ND |
| 173 | B | C | ND | ND | ND |
| 174 | A | A | ND | ND | ND |
| 175 | C | B | ND | ND | ND |
| 176 | A | A | ND | ND | ND |
| 177 | A | A | ND | ND | ND |
| 178 | A | A | ND | ND | ND |
| 179 | A | A | ND | ND | ND |
| 180 | A | A | ND | ND | ND |
| 181 | A | A | ND | ND | ND |
| 182 | A | A | ND | ND | ND |
| 183 | A | A | ND | ND | ND |
| 184 | A | A | ND | ND | ND |
| 185 | B | A | ND | ND | ND |
| 186 | B | B | ND | ND | ND |
| 187 | A | A | ND | ND | ND |
| 188 | A | A | ND | ND | ND |
| 189 | B | B | ND | ND | ND |
| 190 | A | A | ND | ND | ND |
| 191 | A | B | ND | ND | ND |
| 192 | C | D | ND | ND | ND |
| 193 | A | A | ND | ND | ND |
| 194 | A | A | ND | ND | ND |
| 195 | A | A | ND | ND | ND |
| 196 | A | A | ND | ND | ND |
| 197 | A | A | ND | ND | ND |
| 198 | A | A | ND | ND | ND |
| 199 | B | B | ND | ND | ND |
| 200 | C | C | ND | ND | ND |
| 201 | A | C | ND | ND | ND |
| 202 | A | A | ND | ND | ND |
| 203 | A | A | ND | ND | ND |
| 204 | A | A | ND | ND | ND |
| 205 | A | A | ND | ND | ND |
| 206 | A | B | ND | ND | ND |
| 207 | A | B | ND | ND | ND |
| 208 | A | B | ND | ND | ND |
| 209 | A | A | ND | ND | ND |
| 210 | B | B | ND | ND | ND |
| 211 | A | A | ND | ND | ND |
| 212 | A | A | ND | ND | ND |
| 213 | A | A | ND | ND | ND |
| 214 | A | A | ND | ND | ND |
| 215 | B | C | ND | ND | ND |
| 216 | A | A | ND | ND | ND |
| 217 | A | A | ND | ND | ND |
| 218 | A | A | ND | ND | ND |
| 219 | C | C | ND | ND | ND |
| 220 | A | A | ND | ND | ND |
| 221 | A | A | ND | ND | ND |
| 222 | A | A | ND | ND | ND |
| 223 | A | A | ND | ND | ND |
| 224 | A | B | ND | ND | ND |
| 225 | A | A | ND | ND | ND |
| 226 | A | A | ND | ND | ND |
| 228 | A | A | ND | ND | ND |
| 229 | A | A | ND | ND | ND |
| 230 | A | A | ND | ND | ND |
| 231 | A | A | ND | ND | ND |
| 232 | B | B | ND | ND | ND |
| 233 | A | B | ND | ND | ND |
| 234 | A | B | ND | ND | ND |
| 235 | A | A | ND | ND | ND |
| 236 | A | A | ND | ND | ND |
| 237 | A | ND | ND | ND | ND |
| 238 | ND | ND | ND | ND | ND |
| 239 | A | ND | ND | ND | ND |
| 240 | B | C | ND | ND | ND |
| 241 | A | C | ND | ND | ND |
| 242 | A | C | ND | ND | ND |
| 243 | A | A | ND | ND | ND |
| 244 | C | C | ND | ND | ND |
| 245 | A | A | ND | ND | ND |
| 246 | A | A | ND | ND | ND |
| 247 | A | ND | ND | ND | ND |

### Metabolic Stability

Determining the in vitro metabolic stability of a new chemical entity (NCE) is a very useful tool within drug discovery. A metabolic stability study will determine the in vitro intrinsic clearance (CLint) of a compound. The CLint value represents a measure of enzyme activity toward an NCE and is independent of other physiological factors such as blood flow. Determination of CLint within drug discovery helps to identify metabolic liabilities within a compound series, and ultimately assist in the identification of compounds with improved metabolic stability. The CLint value determined in vitro can be used to predict in vivo clearance (using species specific scaling factors) in pre-clinical species and ultimately in human. The liver is the primary organ involved in drug metabolism, of which there are two pathways-Phase I and Phase II. Phase I metabolism is primarily oxidative with the majority of these oxidations catalysed by cytochrome P450 enzymes (CYPs) which are located in the smooth endoplasmic reticulum (ER) in the liver. Phase II metabolism is catalysed by various conjugative enzymes which are located mainly in the ER and the cytosol. The in vitro systems used to study drug metabolism are mainly subcellular fractions such as liver microsomes or whole cells (hepatocytes) isolated from the liver. Hepatocytes, being whole cells, contain both Phase I and Phase II metabolizing enzymes and produce their own cofactors. This assay determines the metabolic stability of test compounds in human liver hepatocytes using the substrate depletion approach. The lower limit of the assay is a T1/2 >462 min and a CLint <5 min. Therefore the upper limit of the assay is 277 μL/min/106 cells. Any CLint value higher than this should be given an out of range value. Hepatocytes were plated in Williams E Media with added L-glutamine, compounds were added as 1 mM DMSO solution and incubated at 37° C. Compound depletion was measured by LC-MS/MS at multiple time points following quenching with cold acetonitrile. CLint was calculated from the following equation:

CLint(μl/min/106cells)=(In(2)/T_{1/2})*1000/cell density(10⁶cells/mL)

τ_{1/2}(min)=In(2)/−slope

As demonstrated in table 30 below compounds of this invention demonstrated lower intrinsic clearance (CLint) (indicative of improved metabolic stability) relative to compounds disclosed in WO2022258974 and WO2022248885).

TABLE 30

| | Human Hepatocyte CLint |
|---|---|
| Compound | (μL/min/10⁶ cells) |
| Example 1 | 73.3 |
| Example 2 | 54.2 |
| WO2022258974 Example 26 | 94.6 |
| Example 9 | 42.5 |
| Example 12 | 47.8 |
| WO2022248885 Example 84 | 95.6 |

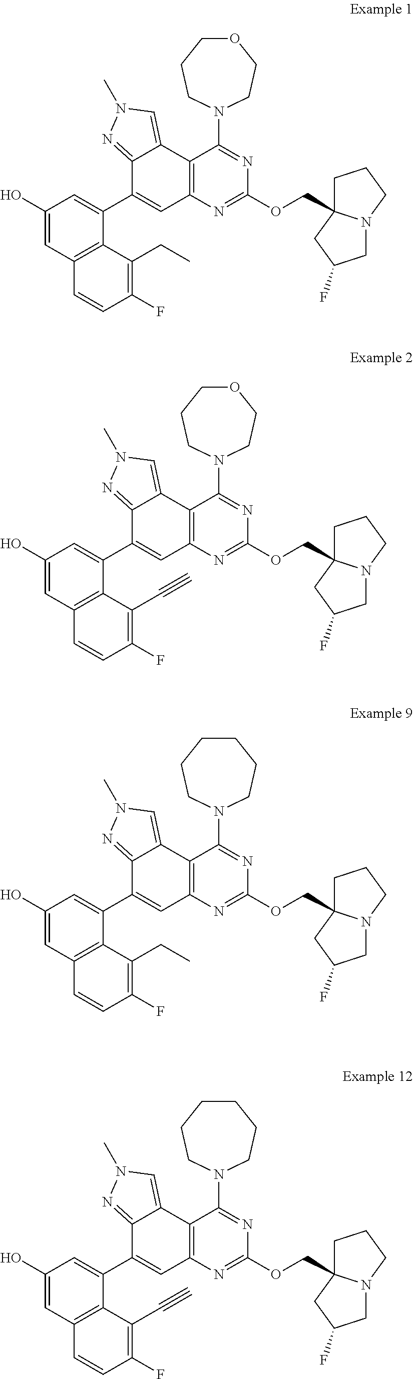 [SMILES: CCc1c(F)ccc2cc(O)cc(-c3cc4nc(OC[C@@]56CCCN5C[C@H](F)C6)nc(N5CCCOCC5)c4c4cn(C)nc34)c12 ; C#Cc1c(F)ccc2cc(O)cc(-c3cc4nc(OC[C@@]56CCCN5C[C@H](F)C6)nc(N5CCCOCC5)c4c4cn(C)nc34)c12 ; CCc1c(F)ccc2cc(O)cc(-c3cc4nc(OC[C@@]56CCCN5C[C@H](F)C6)nc(N5CCCCCC5)c4c4cn(C)nc34)c12 ; C#Cc1c(F)ccc2cc(O)cc(-c3cc4nc(OC[C@@]56CCCN5C[C@H](F)C6)nc(N5CCCCCC5)c4c4cn(C)nc34)c12]

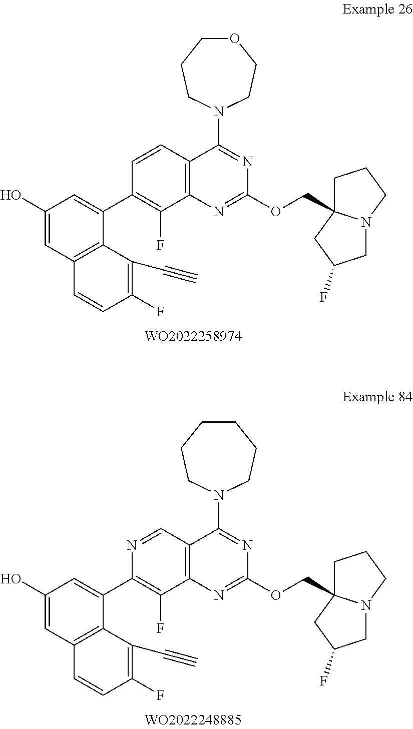 [SMILES: C#Cc1c(F)ccc2cc(O)cc(-c3ccc4c(N5CCCOCC5)nc(OC[C@@]56CCCN5C[C@H](F)C6)nc4c3F)c12 ; C#Cc1c(F)ccc2cc(O)cc(-c3ncc4c(N5CCCCCC5)nc(OC[C@@]56CCCN5C[C@H](F)C6)nc4c3F)c12]

1.-34. (canceled)

35. A compound of formula (I), or a pharmaceutically acceptable salt thereof: 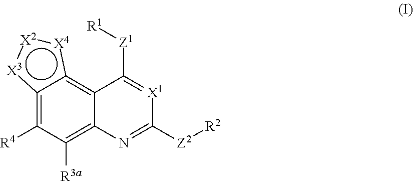
Z¹ is independently selected from —O— and —NR^{5a}—;
Z² is independently absent or is selected from —O— and —NR^{5b}—;
X¹ is independently selected from N and CR^{3b};
X² is selected from N, NR⁶, O and S;
X³ is selected from N and NR⁶;
X⁴ is CR^{3c};
R¹ is independently selected from C₀-C₃-alkylene-R^{1a} and C₂-C₆-alkylene-R^{1b}, wherein Rla is independently selected from: a 4- to 7-membered heterocycloalkyl ring; a phenyl ring; and a C₃-C₇-cycloalkyl ring optionally substituted with an NR⁷R⁸ group; wherein said heterocycloalkyl ring, said phenyl ring or said cycloalkyl ring is optionally substituted with from 1 to 4 R⁹ groups; R^{1b} is independently selected from: NR⁷R⁸, OR⁸, SR⁸, SOR⁸, SO₂R⁸ and SO(NH)R⁸,
or R¹ and R^{5a} together with the nitrogen to which they are attached form a ring system selected from: a monocyclic 4- to 7-membered heterocycloalkyl group, optionally substituted with from 1 to 4 R⁹ groups; and a fused, spirofused or bridged bicyclic 6- to 11-membered heterocyclyl group, optionally substituted with from 1 to 4 R⁹ groups;
R² is independently C₁-C₆-alkyl, C₁-C₄-haloalkyl, C₀-C₄-alkylene-R^{2a}, C₁-C₄-alkylene-R^{2b}, C₂-C₄-alkylene-R^{2c};
R^{2a} is independently selected from monocyclic 4- to 7-membered heterocycloalkyl group; a fused, spirofused or bridged bicyclic 6- to 11-membered heterocycloalkyl group; a 5-, 6-, 9- or 10-membered monocyclic or bicyclic heteroaryl group; phenyl; and C₃-C₇-cycloalkyl; wherein any heterocycloalkyl or cycloalkyl R^{2a} group is optionally substituted with from 1 to 6 R¹⁰ groups and any heteroaryl or phenyl R^{2a} group is optionally substituted with from 1 to 6 R¹¹ groups;
wherein R^{2b} is independently selected from CONR¹²R¹² and CO₂R¹²;
wherein R^{2c} is independently selected from NR¹²R¹³ and OR 12;
or R² and R^{5b} together with the nitrogen to which they are attached form a ring system selected from: monocyclic 4- to 7-membered heterocycloalkyl group; and a fused, spirofused or bridged bicyclic 6- to 11-membered heterocycloalkyl group, said heterocycloalkyl group being optionally substituted with from 1 to 6 R¹⁰ groups;
R^{3a}, R^{3b} and R^{3c} are each independently selected from: H, halo, C₁-C₄-alkyl, O—C₁-C₄-alkyl, C₁-C₄-haloalkyl, O—C₁-C₄-haloalkyl, cyclopropyl, nitro and cyano;
R⁴ is independently selected from phenyl, said phenyl being optionally fused to a C₅-C₇-cycloalkyl ring; naphthyl; monocyclic 4- to 7-membered cycloalkyl or heterocycloalkyl;
and 5-, to 10-membered monocyclic or bicyclic heterocyclyl; wherein R⁴ is optionally substituted with from 1 to 4 R¹⁴ groups;
R^{5a}, R^{5b}, R⁸ and R¹² are each independently selected at each occurrence from H, C₁-C₄-haloalkyl, cyclopropyl and C₁-C₄-alkyl;
R⁶ is independently selected from H, C₁-C₄-haloalkyl, C₁-C₄-alkyl and C₃-C₄-cycloalkyl;
R⁷ and R¹³ are each independently at each occurrence selected from H, C₁-C₄-alkyl, C₁-C₄-haloalkyl and C(O)—C₁-C₄-alkyl;
or R¹² and R¹³ together with the nitrogen to which they are attached form a ring system selected from: monocyclic 4- to 7-membered heterocycloalkyl group; and a fused, spirofused or bridged bicyclic 6- to 11-membered heterocycloalkyl group; said heterocycloalkyl group being optionally substituted with from 1 to 6 R^{10a} groups; R⁹ is independently at each occurrence selected from oxo, halo, cyano, NR¹²R¹³, OR¹², COR¹², CO₂R¹², CONR¹²R¹², CONR¹²R¹³, C₁-C₄-alkyl, C₁-C₄-alkyl substituted with NR¹²R¹³, C₁-C₄-alkyl substituted with OR¹², C₁-C₄-alkyl substituted with cyano, C₂-C₄-alkenyl, C₂-C₄-alkynyl, C₁-C₄-haloalkyl and cyclopropyl;
R¹⁰ is independently at each occurrence selected from oxo, halo, cyano, NR¹²R¹³, OR¹², COR¹², CO₂R¹², CONR¹²R¹², C₁-C₄-alkyl, C₁-C₄-alkyl substituted with NR¹²R¹³, C₁-C₄-alkyl substituted with OR¹², C₁-C₄-alkyl substituted with cyano, C₁-C₄-alkyl substituted with phenyl, C₂-C₄-alkenyl, C₂-C₄-alkynyl, C₁-C₄-haloalkyl and cyclopropyl;
R^{10a} is independently at each occurrence selected from oxo, halo, cyano, NR¹²R^{13a}, OR¹², COR¹², CO₂R¹², CONR¹²R¹², CONR¹²R^{13a}, C₁-C₄-alkyl, C₁-C₄-alkyl substituted with NR¹²R^{13a}, C₁-C₄-alkyl substituted with OR¹², C₁-C₄-alkyl substituted with cyano, C₁-C₄-alkyl substituted with phenyl, C₂-C₄-alkenyl, C₂-C₄-alkynyl, C₁-C₄-haloalkyl and cyclopropyl;
R^{13a} is independently at each occurrence selected from H, C₁-C₄-alkyl, C₁-C₄-haloalkyl and C(O)—C₁-C₄-alkyl;
R¹¹ is independently selected from halo, cyano, nitro, NR¹²R¹³, OR¹², CO₂R¹², CONR¹²R¹², C₁-C₄-alkyl, C₁-C₄-alkyl substituted with NR¹²R¹³, C₁-C₄-alkyl substituted with OR¹², monocyclic 4- to 7-membered cycloalkyl or heterocycloalkyl, C₂-C₄-alkenyl, C₂-C₄-alkynyl, C₁-C₄-haloalkyl and cyclopropyl;
R¹⁴ is independently at each occurrence selected from halo, cyano, nitro, NR¹²R¹³, OR¹², CO₂R¹², CONR¹²R¹², C₁-C₄-alkyl, C₁-C₄-alkyl substituted with NR¹²R¹³, C₁-C₄-alkyl substituted with OR¹², C₂-C₄-alkenyl, C₂-C₄-alkynyl, C₁-C₄-haloalkyl, phenyl and cyclopropyl;
wherein any of the aforementioned alkyl, alkylene, phenyl or cycloalkyl (e.g. cyclopropyl) groups is optionally substituted, where chemically possible, by 1 to 5 substituents which are each independently at each occurrence selected from the group consisting of: C₁-C₄-alkyl, C₁-C₄-alkyl substituted with OR^{a}, halo, nitro, cyano, NR^{a} R^{b}, OR^{a}, SR^{a}, CO₂R^{a}, C(O)R^{a}, CONR^{a}R^{a}; wherein R^{a} is independently at each occurrence selected from H, C₁-C₄-alkyl and C₁-C₄-haloalkyl; and R^{b} is independently at each occurrence selected from H, C₁-C₄-alkyl, C(O)—C₁-C₄-alkyl and S(O)₂—C₁-C₄-alkyl.

36. A compound of claim 35, wherein R¹ and R^{5a} are selected such that NR¹R⁵ comprises no more than a single amine, wherein said single amine may be a primary, secondary or tertiary amine.

37. A compound of claim 35, wherein R¹ and R^{5a} together with the nitrogen to which they are attached form a ring system selected from: a monocyclic 4- to 7-membered heterocycloalkyl group, optionally substituted with from 1 to 4 R⁹ groups; a fused or spirofused bicyclic 6- to 11-membered heterocyclyl group, optionally substituted with from 1 to 4 R⁹ groups; and a bridged bicyclic 6- to 11-membered heterocyclyl group, optionally substituted with from 1 to 4 R⁹ groups; wherein the nitrogen to which R¹ and R^{5a} are attached is the only nitrogen in the ring system.

38. A compound of claim 35, wherein R¹ and R^{5a} together with the nitrogen to which they are attached form a ring system selected from: a monocyclic 4- to 7-membered group heterocycloalkyl group, optionally substituted with from 1 to 4 R⁹ groups; a fused or spirofused bicyclic 6- to 11-membered heterocycloalkyl group, optionally substituted with from 1 to 4 R⁹ groups.

39. A compound of claim 38, wherein R¹ and R⁵ together with the nitrogen to which they are attached form a ring system having the structure: 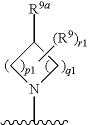
wherein R^{9a} is selected from NR¹²R¹³ and C₁-C₄-alkyl substituted with NR¹²R¹³; p1 is selected from 0, 1, 2 and 3, q1 is selected from 0, 1 and 2; and r1 is selected from 0, 1, 2 and 3.

40. A compound of claim 38, wherein R¹ and R^{5a} together with the nitrogen to which they are attached form a ring system having the structure: 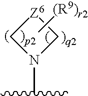
wherein Z⁶ is independently selected from C(O)NR^{9b}, NR^{9b}, O, S, S(O)₂, S(O), S(O)(NR^{9b}) and S(O)(NH); R^{9b} is selected from H and C₁-C₄-alkyl; p2 is selected from 2 and 3, q2 is 2; and r2 is selected from 0, 1, 2 and 3.

41. A compound of claim 39, wherein R¹ and R^{5a} together with the nitrogen to which they are attached form a ring system having the structure: 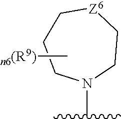
wherein Z⁶ is independently selected from C(O)NR⁹%, O, S, S(O)₂, S(O), S(O)(NR^{9b}), S(O)(NH) and NR^{9b}; R^{9b} is independently at each occurrence selected from H and C₁-C₄-alkyl; and n6 is an integer selected from 0, 1, 2, 3 and 4.

42. A compound of claim 40, wherein Z⁶ may be O.

43. A compound of claim 40, wherein R¹ and R^{5a} together with the nitrogen to which they are attached form a ring system having the structure: 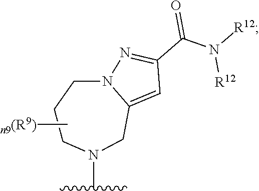
wherein R¹² is independently at each occurrence selected from H, C₁-C₄-haloalkyl, and C₁-C₄-alkyl; and n9 is an integer selected from 0, 1, 2 and 3.

44. A compound of claim 35, wherein R² has the structure: 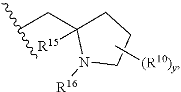
wherein R¹⁵ is independently selected from H, C₁-C₄-alkyl and R¹⁶ is independently selected from H, C₁-C₄-alkyl and cyclopropyl; or wherein R¹⁵ and R¹⁶ together with the atoms to which they are attached form a 5- or 6-membered heterocycloalkyl ring, optionally substituted with 1 or 2 R¹⁰ groups; and y is independently selected from 0, 1, 2, 3, and 4.

45. A compound of claim 44, wherein R² has the structure: 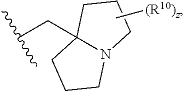
wherein z is independently selected from 0, 1, 2, 3, and 4.

46. A compound of claim 35, wherein X¹ is N.

47. A compound of claim 35, wherein X² is N and X³ is NR⁶.

48. A compound of claim 35, wherein X³ is N and X² is NR⁶.

49. A compound of claim 47, wherein R⁶ is selected from C₁-C₄-haloalkyl, C₁-C₄-alkyl and C₃-C₄-cycloalkyl.

50. A compound of claim 35, wherein R^{3c} is H.

51. A compound of claim 35, wherein R^{3a} is H.

52. A compound of claim 35, wherein R^{3a} is F.

53. A compound of claim 35, wherein R^{3a} is methyl.

54. A compound of claim 35, wherein R⁴ is phenyl, said phenyl being optionally fused to a C₅-C₇-cycloalkyl ring, wherein R⁴ is optionally substituted with from 1 to 4 R¹⁴ groups.

55. A compound of claim 35, wherein R⁴ has the structure: 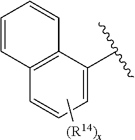
wherein x is independently selected from 0, 1, 2, 3, and 4.

56. A compound of claim 55, wherein R⁴ has the structure: 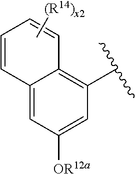
wherein R^{12a} is independently H or C₁-C₄-alkyl; x2 is independently selected from 0, 1, 2 and 3.

57. A compound of claim 35 having the structure:

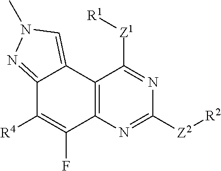

58. A compound of claim 35 having the structure:

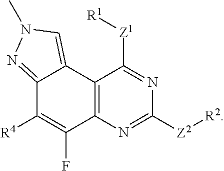

59. A compound of claim 35 having the structure:

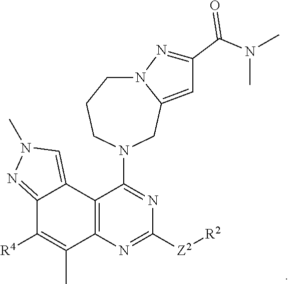

60. A compound of claim 35 having the structure:

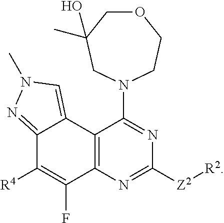

61. A compound of claim 35 having the structure

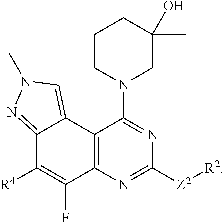

62. A compound of claim 35, wherein R⁴ is 5-, 6-, 9- or 10-membered monocyclic or bicyclic heteroaryl, optionally substituted with from 1 to 4 R¹⁴ groups.

63. A compound of claim 35, wherein the compound of formula (I) is selected from:

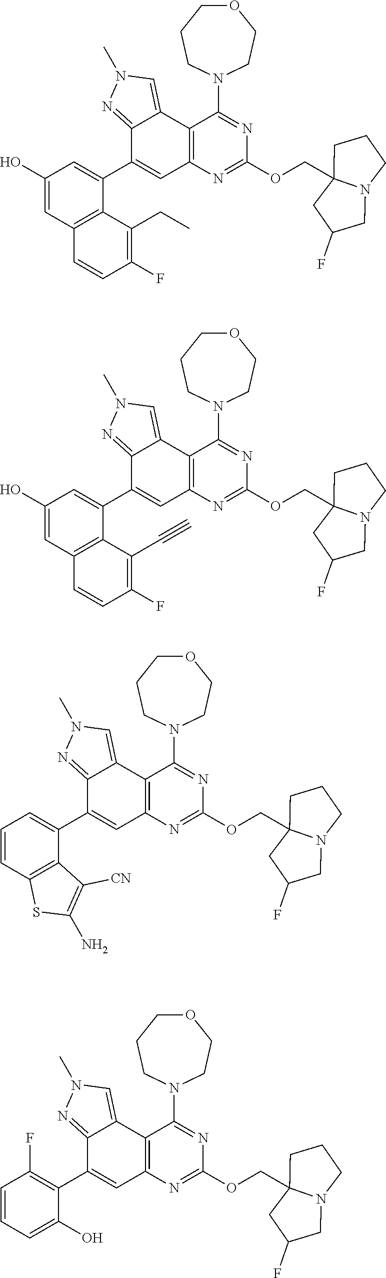 [SMILES: CCc1c(F)ccc2cc(O)cc(-c3cc4nc(OCC56CCCN5CC(F)C6)nc(N5CCCOCC5)c4c4cn(C)nc34)c12 ; C#Cc1c(F)ccc2cc(O)cc(-c3cc4nc(OCC56CCCN5CC(F)C6)nc(N5CCCOCC5)c4c4cn(C)nc34)c12 ; Cn1cc2c(n1)c(-c1cccc3sc(N)c(C#N)c13)cc1nc(OCC34CCCN3CC(F)C4)nc(N3CCCOCC3)c12 ; Cn1cc2c(n1)c(-c1c(O)cccc1F)cc1nc(OCC34CCCN3CC(F)C4)nc(N3CCCOCC3)c12]

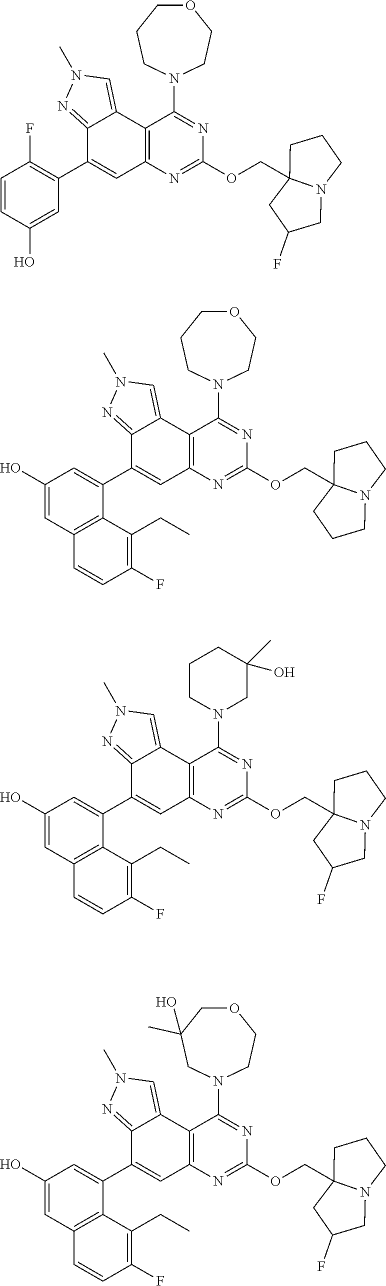 [SMILES: Cn1cc2c(n1)c(-c1cc(O)ccc1F)cc1nc(OCC34CCCN3CC(F)C4)nc(N3CCCOCC3)c12 ; CCc1c(F)ccc2cc(O)cc(-c3cc4nc(OCC56CCCN5CCC6)nc(N5CCCOCC5)c4c4cn(C)nc34)c12 ; CCc1c(F)ccc2cc(O)cc(-c3cc4nc(OCC56CCCN5CC(F)C6)nc(N5CCCC(C)(O)C5)c4c4cn(C)nc34)c12 ; CCc1c(F)ccc2cc(O)cc(-c3cc4nc(OCC56CCCN5CC(F)C6)nc(N5CCOCC(C)(O)C5)c4c4cn(C)nc34)c12]

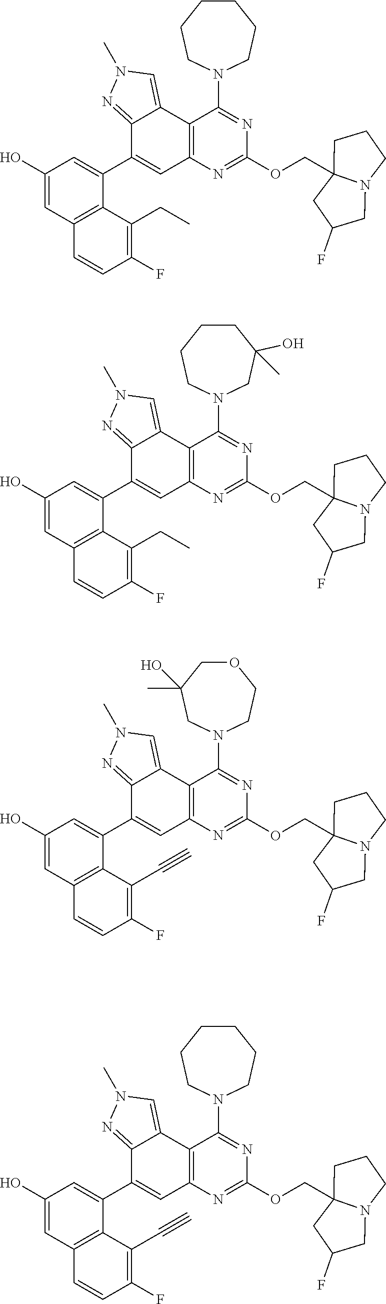 [SMILES: CCc1c(F)ccc2cc(O)cc(-c3cc4nc(OCC56CCCN5CC(F)C6)nc(N5CCCCCC5)c4c4cn(C)nc34)c12 ; CCc1c(F)ccc2cc(O)cc(-c3cc4nc(OCC56CCCN5CC(F)C6)nc(N5CCCCC(C)(O)C5)c4c4cn(C)nc34)c12 ; C#Cc1c(F)ccc2cc(O)cc(-c3cc4nc(OCC56CCCN5CC(F)C6)nc(N5CCOCC(C)(O)C5)c4c4cn(C)nc34)c12 ; C#Cc1c(F)ccc2cc(O)cc(-c3cc4nc(OCC56CCCN5CC(F)C6)nc(N5CCCCCC5)c4c4cn(C)nc34)c12]

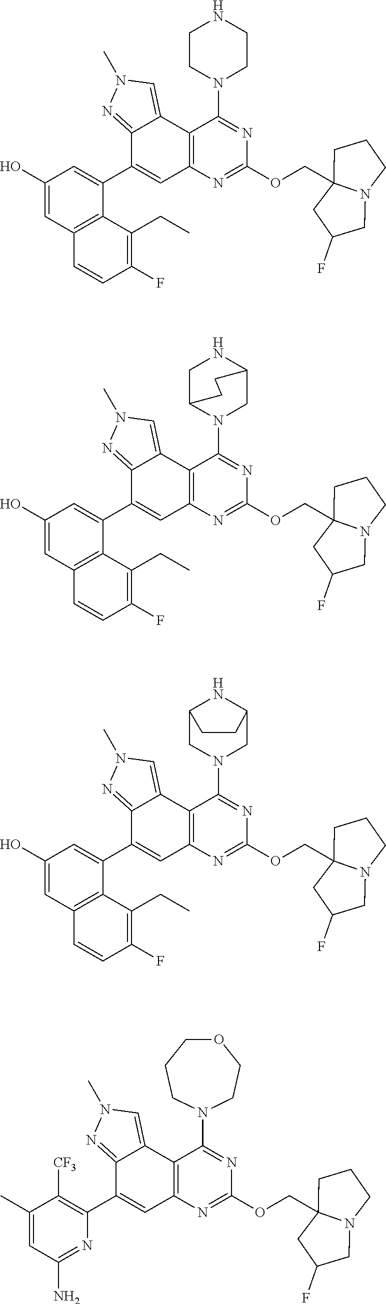 [SMILES: CCc1c(F)ccc2cc(O)cc(-c3cc4nc(OCC56CCCN5CC(F)C6)nc(N5CCNCC5)c4c4cn(C)nc34)c12 ; CCc1c(F)ccc2cc(O)cc(-c3cc4nc(OCC56CCCN5CC(F)C6)nc(N5CC6CCC5CN6)c4c4cn(C)nc34)c12 ; CCc1c(F)ccc2cc(O)cc(-c3cc4nc(OCC56CCCN5CC(F)C6)nc(N5CC6CCC(C5)N6)c4c4cn(C)nc34)c12 ; Cc1cc(N)nc(-c2cc3nc(OCC45CCCN4CC(F)C5)nc(N4CCCOCC4)c3c3cn(C)nc23)c1C(F)(F)F]

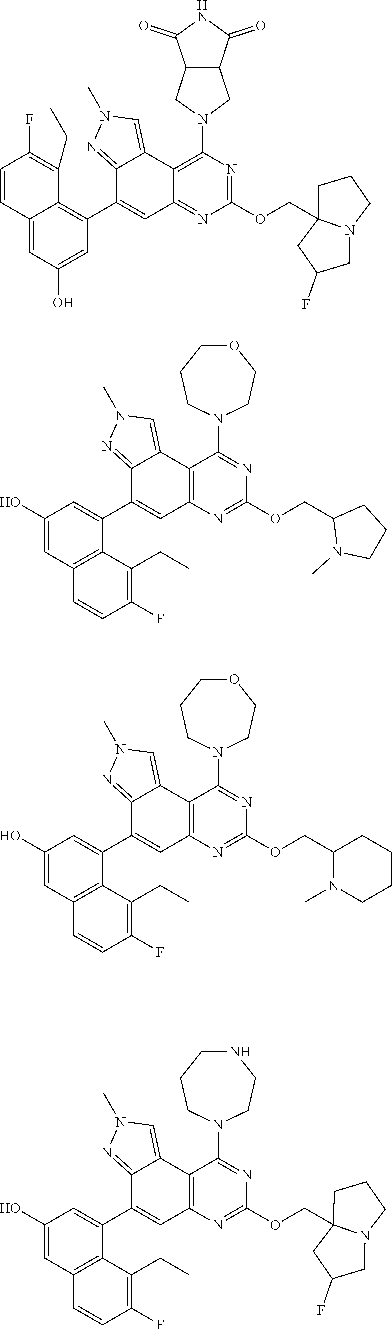 [SMILES: CCc1c(F)ccc2cc(O)cc(-c3cc4nc(OCC56CCCN5CC(F)C6)nc(N5CC6C(=O)NC(=O)C6C5)c4c4cn(C)nc34)c12 ; CCc1c(F)ccc2cc(O)cc(-c3cc4nc(OCC5CCCN5C)nc(N5CCCOCC5)c4c4cn(C)nc34)c12 ; CCc1c(F)ccc2cc(O)cc(-c3cc4nc(OCC5CCCCN5C)nc(N5CCCOCC5)c4c4cn(C)nc34)c12 ; CCc1c(F)ccc2cc(O)cc(-c3cc4nc(OCC56CCCN5CC(F)C6)nc(N5CCCNCC5)c4c4cn(C)nc34)c12]

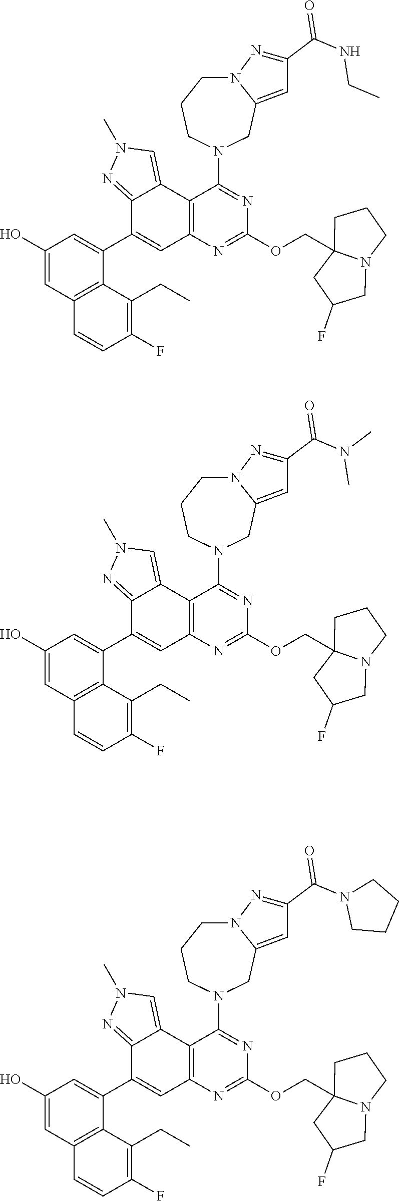 [SMILES: CCNC(=O)c1cc2n(n1)CCCN(c1nc(OCC34CCCN3CC(F)C4)nc3cc(-c4cc(O)cc5ccc(F)c(CC)c45)c4nn(C)cc4c13)C2 ; CCc1c(F)ccc2cc(O)cc(-c3cc4nc(OCC56CCCN5CC(F)C6)nc(N5CCCn6nc(C(=O)N(C)C)cc6C5)c4c4cn(C)nc34)c12 ; CCc1c(F)ccc2cc(O)cc(-c3cc4nc(OCC56CCCN5CC(F)C6)nc(N5CCCn6nc(C(=O)N7CCCC7)cc6C5)c4c4cn(C)nc34)c12]

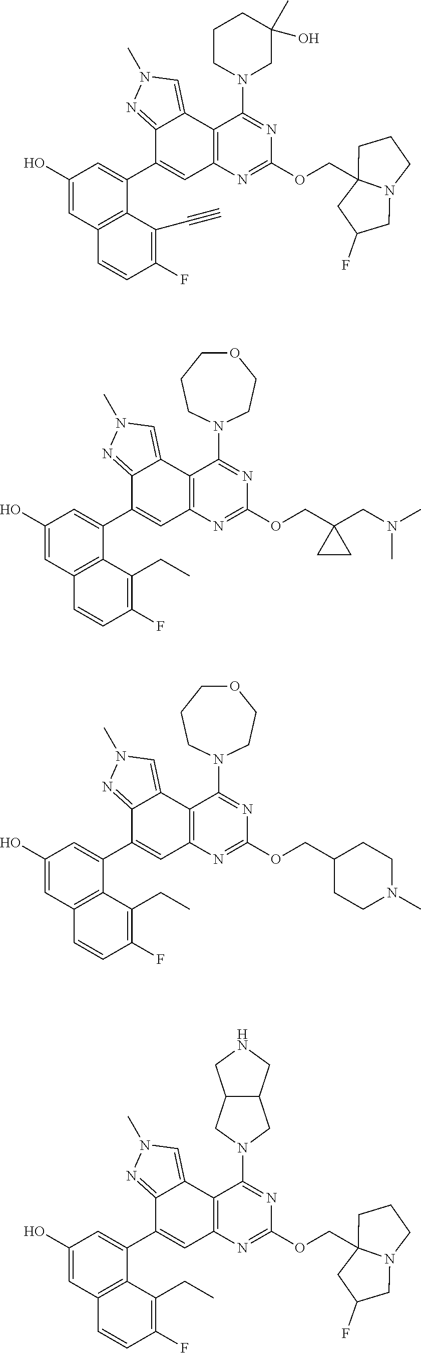 [SMILES: C#Cc1c(F)ccc2cc(O)cc(-c3cc4nc(OCC56CCCN5CC(F)C6)nc(N5CCCC(C)(O)C5)c4c4cn(C)nc34)c12 ; CCc1c(F)ccc2cc(O)cc(-c3cc4nc(OCC5(CN(C)C)CC5)nc(N5CCCOCC5)c4c4cn(C)nc34)c12 ; CCc1c(F)ccc2cc(O)cc(-c3cc4nc(OCC5CCN(C)CC5)nc(N5CCCOCC5)c4c4cn(C)nc34)c12 ; CCc1c(F)ccc2cc(O)cc(-c3cc4nc(OCC56CCCN5CC(F)C6)nc(N5CC6CNCC6C5)c4c4cn(C)nc34)c12]

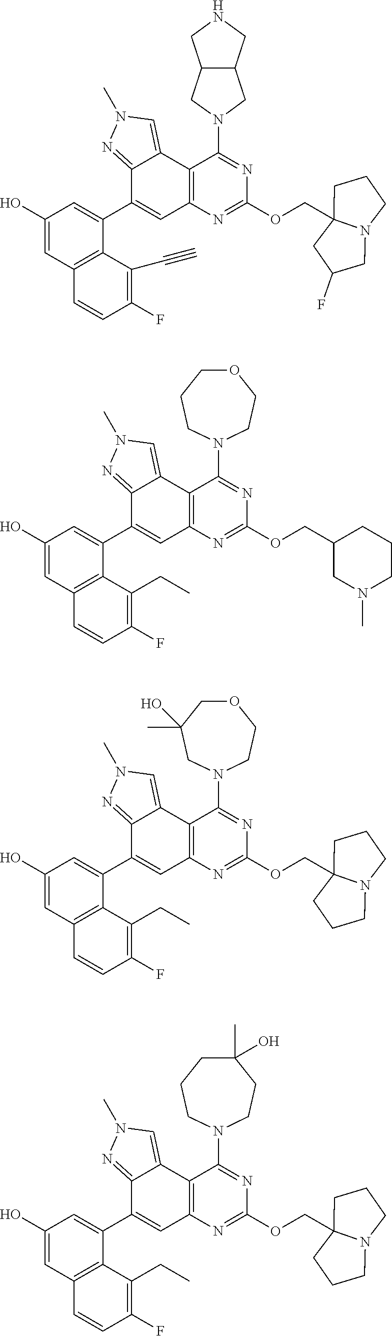 [SMILES: C#Cc1c(F)ccc2cc(O)cc(-c3cc4nc(OCC56CCCN5CC(F)C6)nc(N5CC6CNCC6C5)c4c4cn(C)nc34)c12 ; CCc1c(F)ccc2cc(O)cc(-c3cc4nc(OCC5CCCN(C)C5)nc(N5CCCOCC5)c4c4cn(C)nc34)c12 ; CCc1c(F)ccc2cc(O)cc(-c3cc4nc(OCC56CCCN5CCC6)nc(N5CCOCC(C)(O)C5)c4c4cn(C)nc34)c12 ; CCc1c(F)ccc2cc(O)cc(-c3cc4nc(OCC56CCCN5CCC6)nc(N5CCCC(C)(O)CC5)c4c4cn(C)nc34)c12]

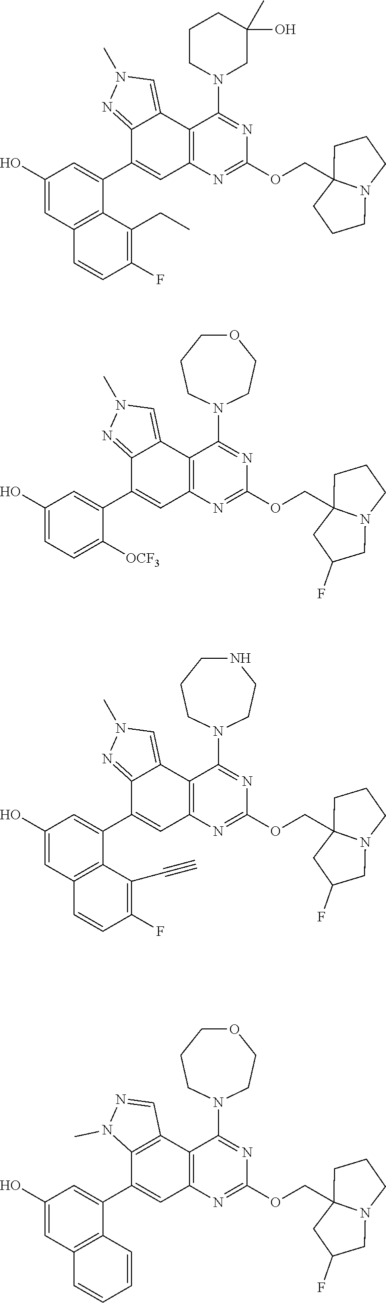 [SMILES: CCc1c(F)ccc2cc(O)cc(-c3cc4nc(OCC56CCCN5CCC6)nc(N5CCCC(C)(O)C5)c4c4cn(C)nc34)c12 ; Cn1cc2c(n1)c(-c1cc(O)ccc1OC(F)(F)F)cc1nc(OCC34CCCN3CC(F)C4)nc(N3CCCOCC3)c12 ; C#Cc1c(F)ccc2cc(O)cc(-c3cc4nc(OCC56CCCN5CC(F)C6)nc(N5CCCNCC5)c4c4cn(C)nc34)c12 ; Cn1ncc2c3c(N4CCCOCC4)nc(OCC45CCCN4CC(F)C5)nc3cc(-c3cc(O)cc4ccccc34)c21]

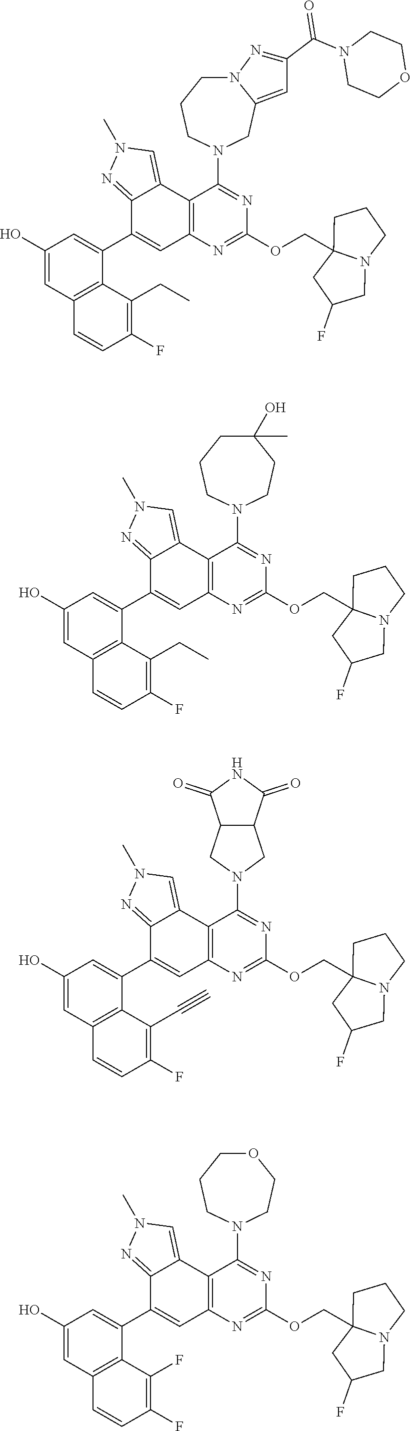 [SMILES: CCc1c(F)ccc2cc(O)cc(-c3cc4nc(OCC56CCCN5CC(F)C6)nc(N5CCCn6nc(C(=O)N7CCOCC7)cc6C5)c4c4cn(C)nc34)c12 ; CCc1c(F)ccc2cc(O)cc(-c3cc4nc(OCC56CCCN5CC(F)C6)nc(N5CCCC(C)(O)CC5)c4c4cn(C)nc34)c12 ; C#Cc1c(F)ccc2cc(O)cc(-c3cc4nc(OCC56CCCN5CC(F)C6)nc(N5CC6C(=O)NC(=O)C6C5)c4c4cn(C)nc34)c12 ; Cn1cc2c(n1)c(-c1cc(O)cc3ccc(F)c(F)c13)cc1nc(OCC34CCCN3CC(F)C4)nc(N3CCCOCC3)c12]

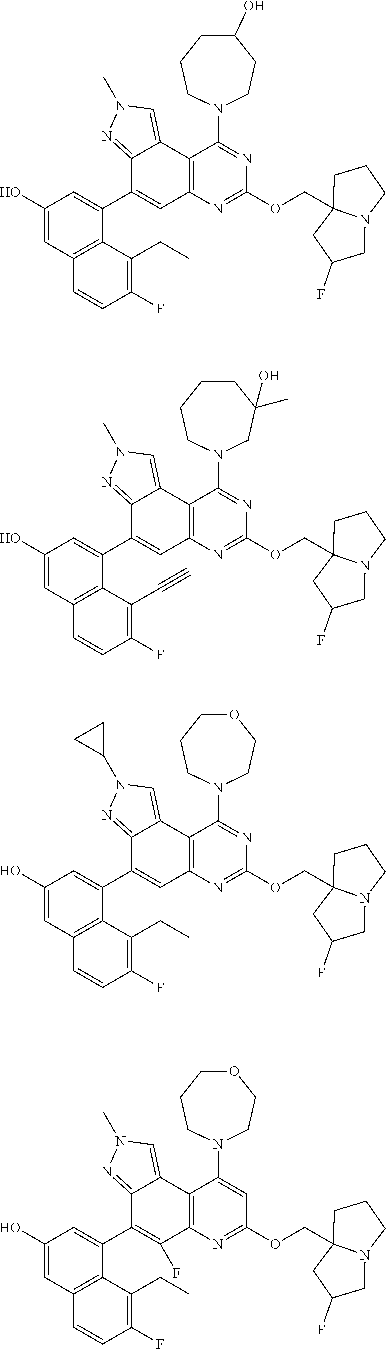 [SMILES: CCc1c(F)ccc2cc(O)cc(-c3cc4nc(OCC56CCCN5CC(F)C6)nc(N5CCCC(O)CC5)c4c4cn(C)nc34)c12 ; C#Cc1c(F)ccc2cc(O)cc(-c3cc4nc(OCC56CCCN5CC(F)C6)nc(N5CCCCC(C)(O)C5)c4c4cn(C)nc34)c12 ; CCc1c(F)ccc2cc(O)cc(-c3cc4nc(OCC56CCCN5CC(F)C6)nc(N5CCCOCC5)c4c4cn(C5CC5)nc34)c12 ; CCc1c(F)ccc2cc(O)cc(-c3c(F)c4nc(OCC56CCCN5CC(F)C6)cc(N5CCCOCC5)c4c4cn(C)nc34)c12]

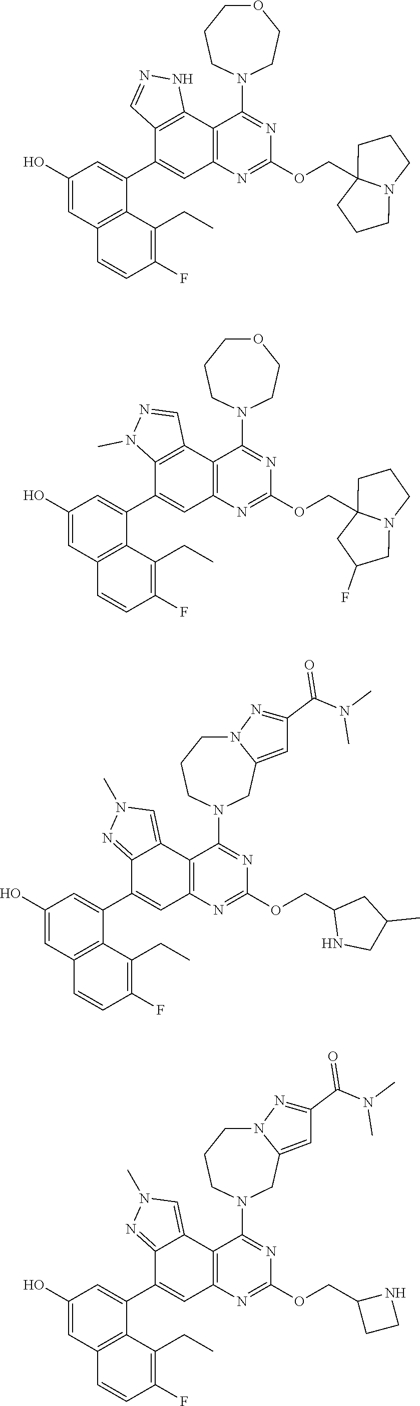 [SMILES: CCc1c(F)ccc2cc(O)cc(-c3cc4nc(OCC56CCCN5CCC6)nc(N5CCCOCC5)c4c4[nH]ncc34)c12 ; CCc1c(F)ccc2cc(O)cc(-c3cc4nc(OCC56CCCN5CC(F)C6)nc(N5CCCOCC5)c4c4cnn(C)c34)c12 ; CCc1c(F)ccc2cc(O)cc(-c3cc4nc(OCC5CC(C)CN5)nc(N5CCCn6nc(C(=O)N(C)C)cc6C5)c4c4cn(C)nc34)c12 ; CCc1c(F)ccc2cc(O)cc(-c3cc4nc(OCC5CCN5)nc(N5CCCn6nc(C(=O)N(C)C)cc6C5)c4c4cn(C)nc34)c12]

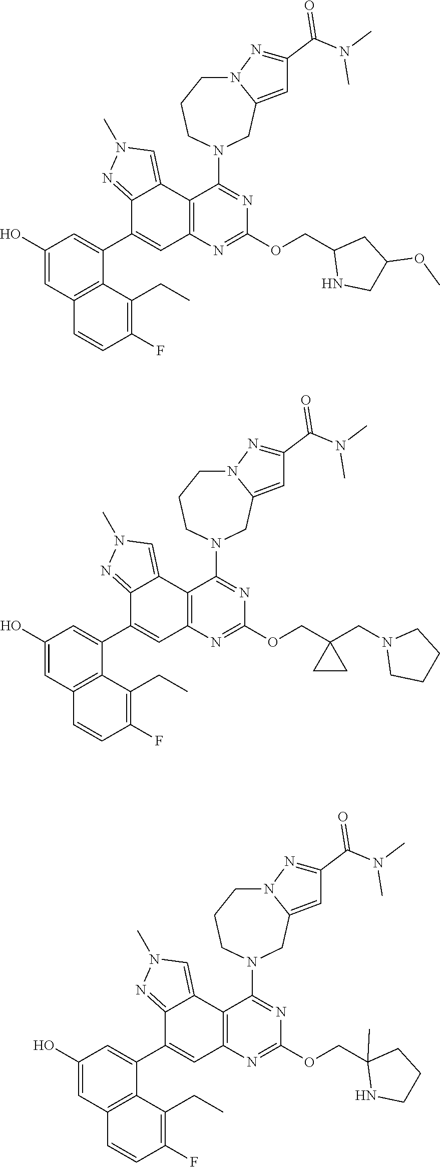 [SMILES: CCc1c(F)ccc2cc(O)cc(-c3cc4nc(OCC5CC(OC)CN5)nc(N5CCCn6nc(C(=O)N(C)C)cc6C5)c4c4cn(C)nc34)c12 ; CCc1c(F)ccc2cc(O)cc(-c3cc4nc(OCC5(CN6CCCC6)CC5)nc(N5CCCn6nc(C(=O)N(C)C)cc6C5)c4c4cn(C)nc34)c12 ; CCc1c(F)ccc2cc(O)cc(-c3cc4nc(OCC5(C)CCCN5)nc(N5CCCn6nc(C(=O)N(C)C)cc6C5)c4c4cn(C)nc34)c12]

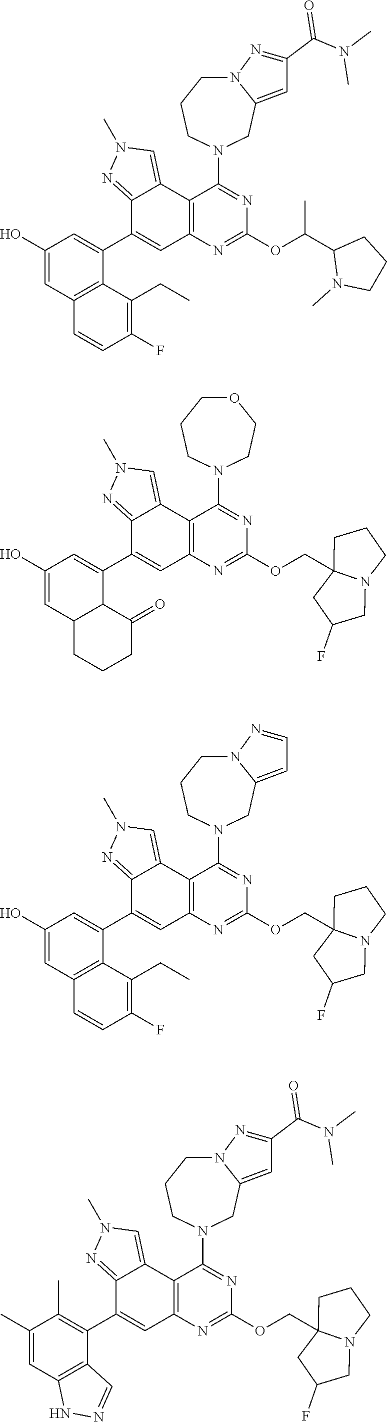 [SMILES: CCc1c(F)ccc2cc(O)cc(-c3cc4nc(OC(C)C5CCCN5C)nc(N5CCCn6nc(C(=O)N(C)C)cc6C5)c4c4cn(C)nc34)c12 ; Cn1cc2c(n1)c(C1=CC(O)=CC3CCCC(=O)C13)cc1nc(OCC34CCCN3CC(F)C4)nc(N3CCCOCC3)c12 ; CCc1c(F)ccc2cc(O)cc(-c3cc4nc(OCC56CCCN5CC(F)C6)nc(N5CCCn6nccc6C5)c4c4cn(C)nc34)c12 ; Cc1cc2[nH]ncc2c(-c2cc3nc(OCC45CCCN4CC(F)C5)nc(N4CCCn5nc(C(=O)N(C)C)cc5C4)c3c3cn(C)nc23)c1C]

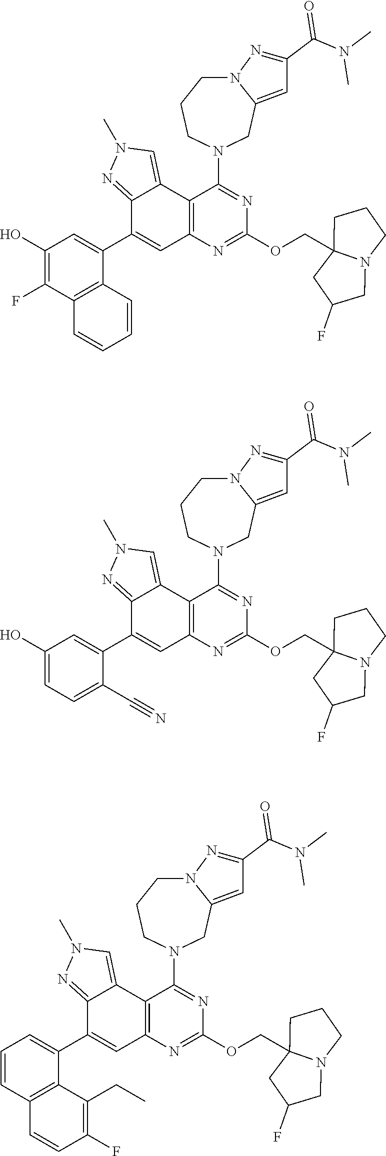 [SMILES: CN(C)C(=O)c1cc2n(n1)CCCN(c1nc(OCC34CCCN3CC(F)C4)nc3cc(-c4cc(O)c(F)c5ccccc45)c4nn(C)cc4c13)C2 ; CN(C)C(=O)c1cc2n(n1)CCCN(c1nc(OCC34CCCN3CC(F)C4)nc3cc(-c4cc(O)ccc4C#N)c4nn(C)cc4c13)C2 ; CCc1c(F)ccc2cccc(-c3cc4nc(OCC56CCCN5CC(F)C6)nc(N5CCCn6nc(C(=O)N(C)C)cc6C5)c4c4cn(C)nc34)c12]

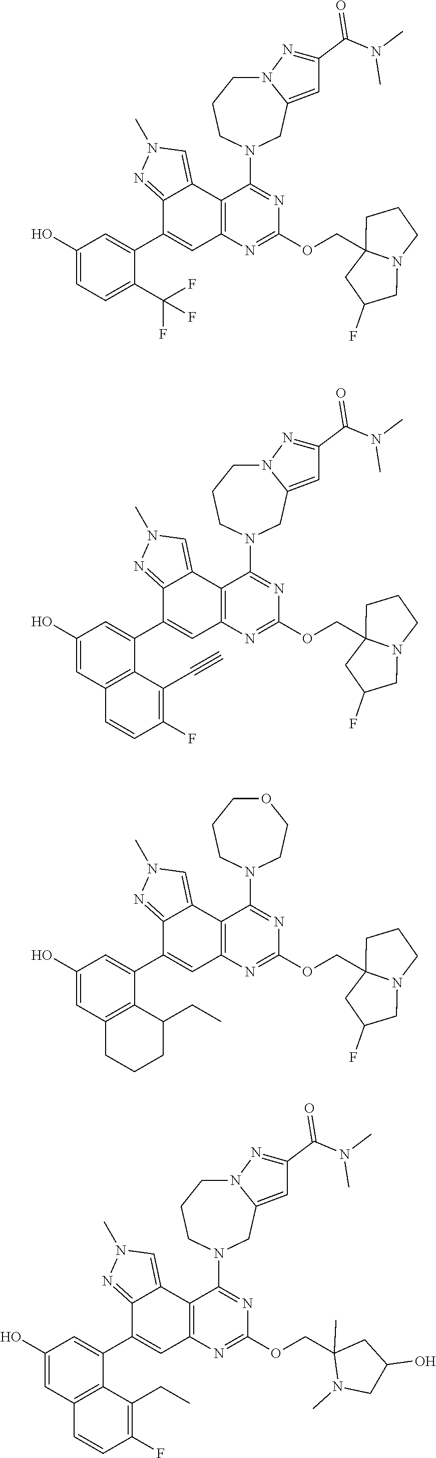 [SMILES: CN(C)C(=O)c1cc2n(n1)CCCN(c1nc(OCC34CCCN3CC(F)C4)nc3cc(-c4cc(O)ccc4C(F)(F)F)c4nn(C)cc4c13)C2 ; C#Cc1c(F)ccc2cc(O)cc(-c3cc4nc(OCC56CCCN5CC(F)C6)nc(N5CCCn6nc(C(=O)N(C)C)cc6C5)c4c4cn(C)nc34)c12 ; CCC1CCCc2cc(O)cc(-c3cc4nc(OCC56CCCN5CC(F)C6)nc(N5CCCOCC5)c4c4cn(C)nc34)c21 ; CCc1c(F)ccc2cc(O)cc(-c3cc4nc(OCC5(C)CC(O)CN5C)nc(N5CCCn6nc(C(=O)N(C)C)cc6C5)c4c4cn(C)nc34)c12]

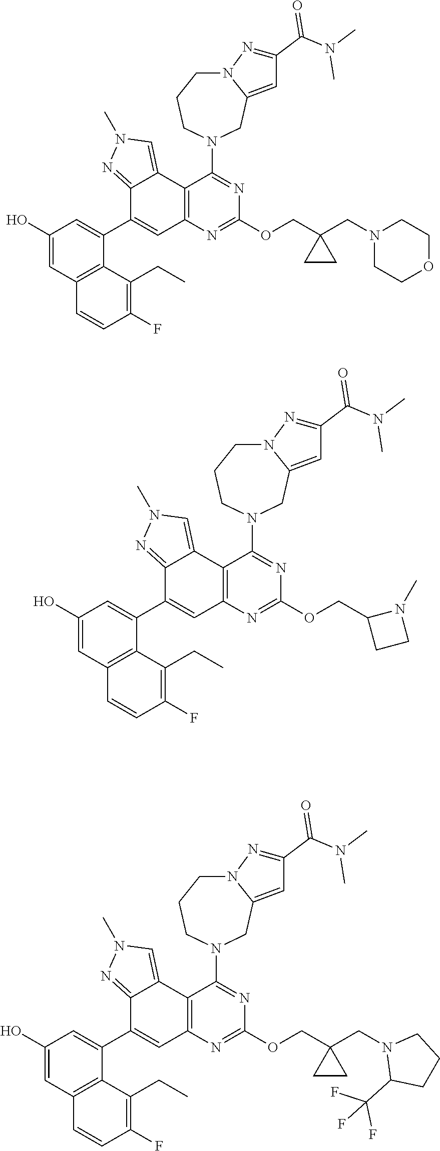 [SMILES: CCc1c(F)ccc2cc(O)cc(-c3cc4nc(OCC5(CN6CCOCC6)CC5)nc(N5CCCn6nc(C(=O)N(C)C)cc6C5)c4c4cn(C)nc34)c12 ; CCc1c(F)ccc2cc(O)cc(-c3cc4nc(OCC5CCN5C)nc(N5CCCn6nc(C(=O)N(C)C)cc6C5)c4c4cn(C)nc34)c12 ; CCc1c(F)ccc2cc(O)cc(-c3cc4nc(OCC5(CN6CCCC6C(F)(F)F)CC5)nc(N5CCCn6nc(C(=O)N(C)C)cc6C5)c4c4cn(C)nc34)c12]

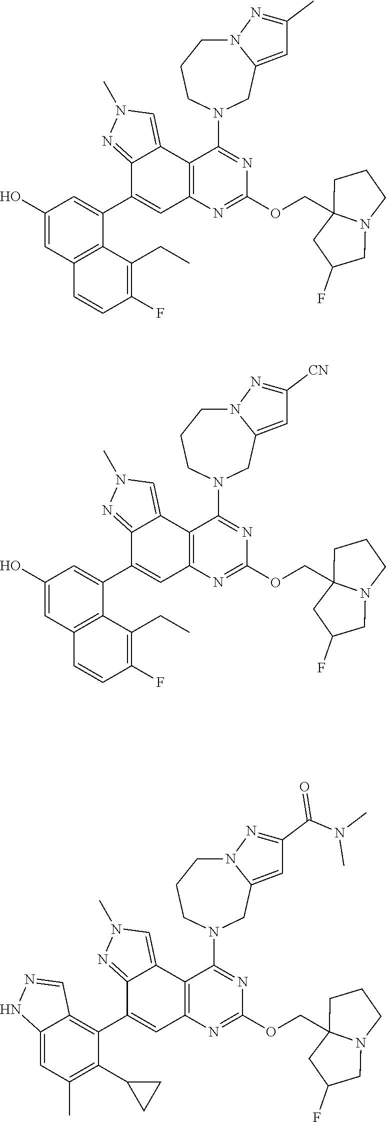 [SMILES: CCc1c(F)ccc2cc(O)cc(-c3cc4nc(OCC56CCCN5CC(F)C6)nc(N5CCCn6nc(C)cc6C5)c4c4cn(C)nc34)c12 ; CCc1c(F)ccc2cc(O)cc(-c3cc4nc(OCC56CCCN5CC(F)C6)nc(N5CCCn6nc(C#N)cc6C5)c4c4cn(C)nc34)c12 ; Cc1cc2[nH]ncc2c(-c2cc3nc(OCC45CCCN4CC(F)C5)nc(N4CCCn5nc(C(=O)N(C)C)cc5C4)c3c3cn(C)nc23)c1C1CC1]

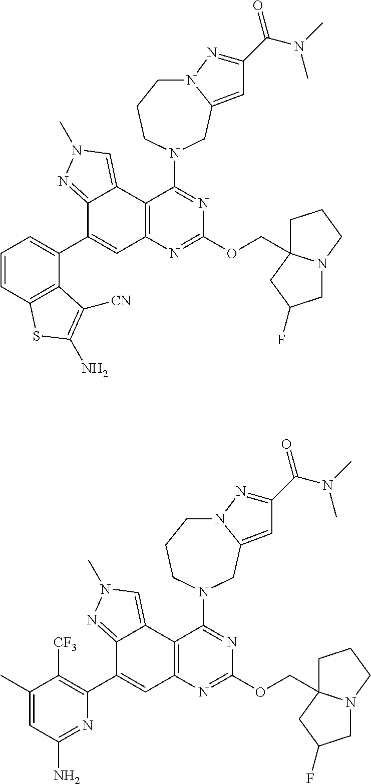 [SMILES: CN(C)C(=O)c1cc2n(n1)CCCN(c1nc(OCC34CCCN3CC(F)C4)nc3cc(-c4cccc5sc(N)c(C#N)c45)c4nn(C)cc4c13)C2 ; Cc1cc(N)nc(-c2cc3nc(OCC45CCCN4CC(F)C5)nc(N4CCCn5nc(C(=O)N(C)C)cc5C4)c3c3cn(C)nc23)c1C(F)(F)F]

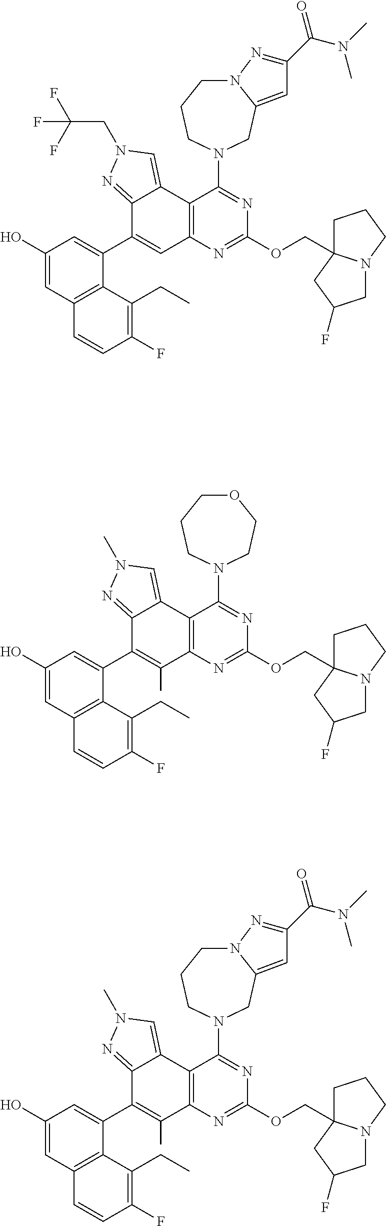 [SMILES: CCc1c(F)ccc2cc(O)cc(-c3cc4nc(OCC56CCCN5CC(F)C6)nc(N5CCCn6nc(C(=O)N(C)C)cc6C5)c4c4cn(CC(F)(F)F)nc34)c12 ; CCc1c(F)ccc2cc(O)cc(-c3c(C)c4nc(OCC56CCCN5CC(F)C6)nc(N5CCCOCC5)c4c4cn(C)nc34)c12 ; CCc1c(F)ccc2cc(O)cc(-c3c(C)c4nc(OCC56CCCN5CC(F)C6)nc(N5CCCn6nc(C(=O)N(C)C)cc6C5)c4c4cn(C)nc34)c12]

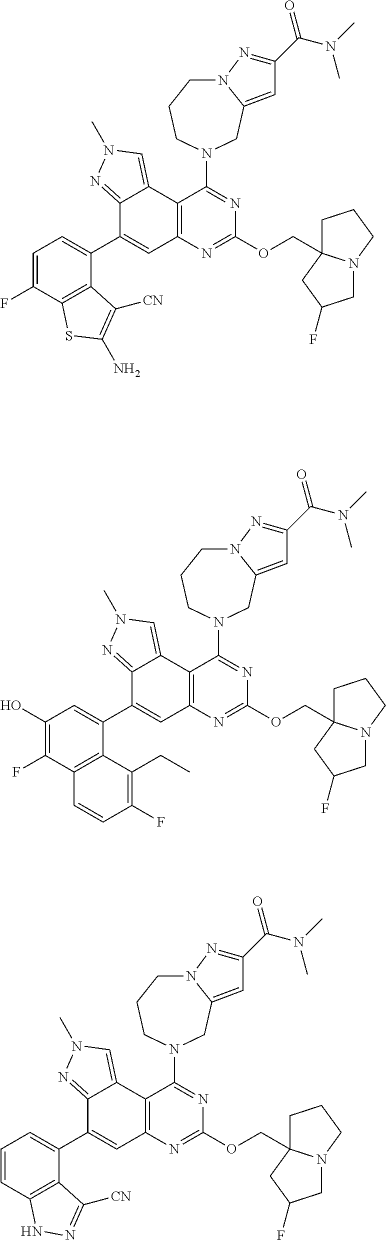 [SMILES: CN(C)C(=O)c1cc2n(n1)CCCN(c1nc(OCC34CCCN3CC(F)C4)nc3cc(-c4ccc(F)c5sc(N)c(C#N)c45)c4nn(C)cc4c13)C2 ; CCc1c(F)ccc2c(F)c(O)cc(-c3cc4nc(OCC56CCCN5CC(F)C6)nc(N5CCCn6nc(C(=O)N(C)C)cc6C5)c4c4cn(C)nc34)c12 ; CN(C)C(=O)c1cc2n(n1)CCCN(c1nc(OCC34CCCN3CC(F)C4)nc3cc(-c4cccc5[nH]nc(C#N)c45)c4nn(C)cc4c13)C2]

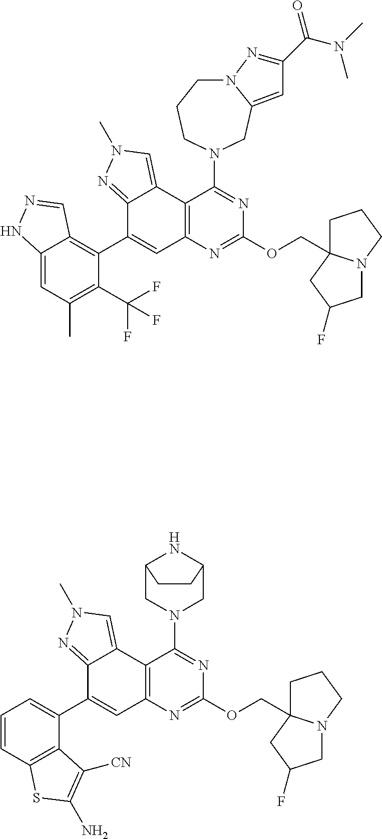 [SMILES: Cc1cc2[nH]ncc2c(-c2cc3nc(OCC45CCCN4CC(F)C5)nc(N4CCCn5nc(C(=O)N(C)C)cc5C4)c3c3cn(C)nc23)c1C(F)(F)F ; Cn1cc2c(n1)c(-c1cccc3sc(N)c(C#N)c13)cc1nc(OCC34CCCN3CC(F)C4)nc(N3CC4CCC(C3)N4)c12]

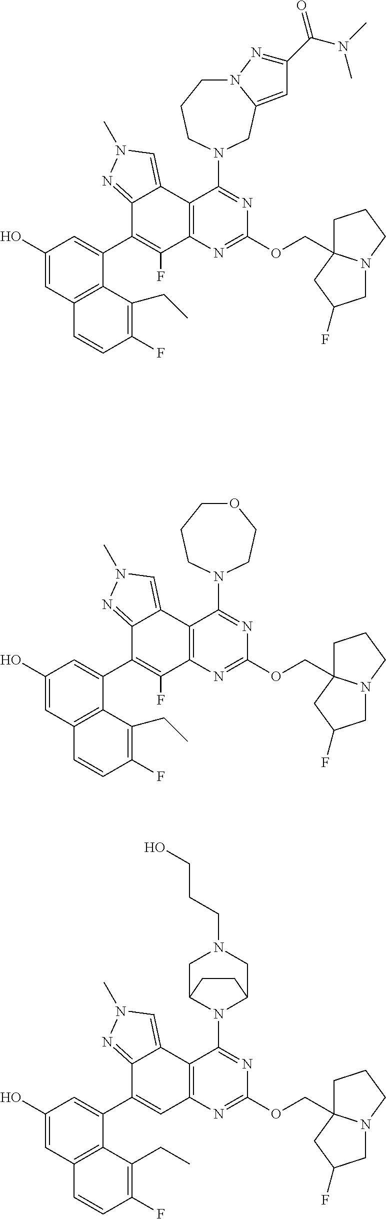 [SMILES: CCc1c(F)ccc2cc(O)cc(-c3c(F)c4nc(OCC56CCCN5CC(F)C6)nc(N5CCCn6nc(C(=O)N(C)C)cc6C5)c4c4cn(C)nc34)c12 ; CCc1c(F)ccc2cc(O)cc(-c3c(F)c4nc(OCC56CCCN5CC(F)C6)nc(N5CCCOCC5)c4c4cn(C)nc34)c12 ; CCc1c(F)ccc2cc(O)cc(-c3cc4nc(OCC56CCCN5CC(F)C6)nc(N5C6CCC5CN(CCCO)C6)c4c4cn(C)nc34)c12]

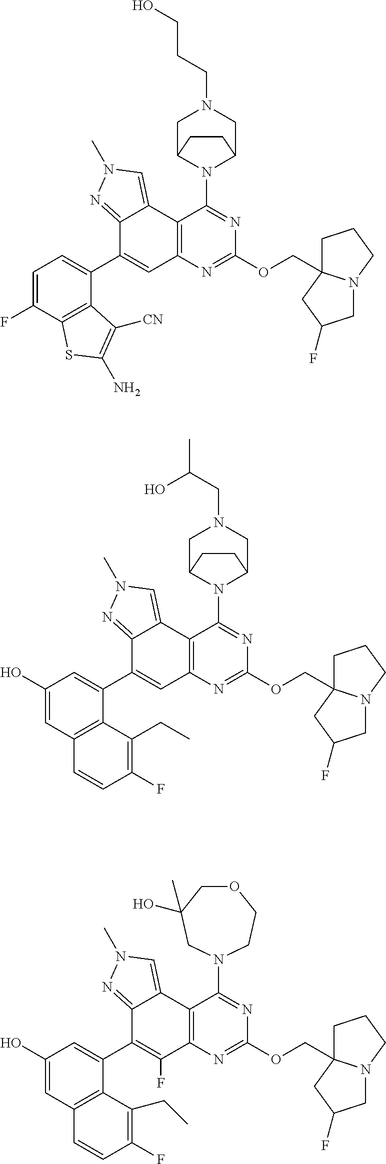 [SMILES: Cn1cc2c(n1)c(-c1ccc(F)c3sc(N)c(C#N)c13)cc1nc(OCC34CCCN3CC(F)C4)nc(N3C4CCC3CN(CCCO)C4)c12 ; CCc1c(F)ccc2cc(O)cc(-c3cc4nc(OCC56CCCN5CC(F)C6)nc(N5C6CCC5CN(CC(C)O)C6)c4c4cn(C)nc34)c12 ; CCc1c(F)ccc2cc(O)cc(-c3c(F)c4nc(OCC56CCCN5CC(F)C6)nc(N5CCOCC(C)(O)C5)c4c4cn(C)nc34)c12]

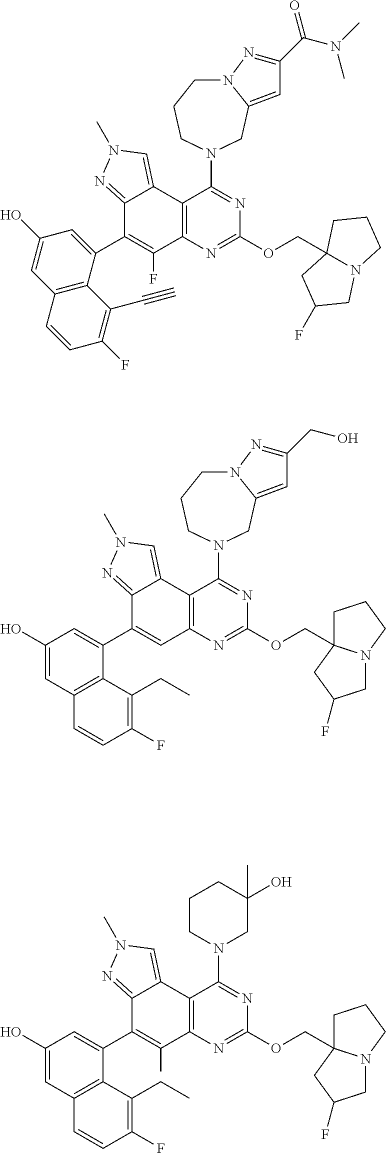 [SMILES: C#Cc1c(F)ccc2cc(O)cc(-c3c(F)c4nc(OCC56CCCN5CC(F)C6)nc(N5CCCn6nc(C(=O)N(C)C)cc6C5)c4c4cn(C)nc34)c12 ; CCc1c(F)ccc2cc(O)cc(-c3cc4nc(OCC56CCCN5CC(F)C6)nc(N5CCCn6nc(CO)cc6C5)c4c4cn(C)nc34)c12 ; CCc1c(F)ccc2cc(O)cc(-c3c(C)c4nc(OCC56CCCN5CC(F)C6)nc(N5CCCC(C)(O)C5)c4c4cn(C)nc34)c12]

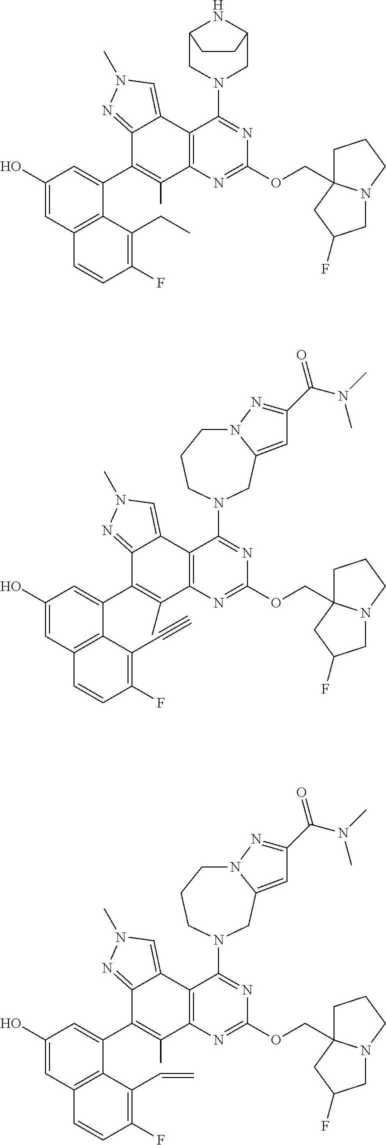 [SMILES: CCc1c(F)ccc2cc(O)cc(-c3c(C)c4nc(OCC56CCCN5CC(F)C6)nc(N5CC6CCC(C5)N6)c4c4cn(C)nc34)c12 ; C#Cc1c(F)ccc2cc(O)cc(-c3c(C)c4nc(OCC56CCCN5CC(F)C6)nc(N5CCCn6nc(C(=O)N(C)C)cc6C5)c4c4cn(C)nc34)c12 ; C=Cc1c(F)ccc2cc(O)cc(-c3c(C)c4nc(OCC56CCCN5CC(F)C6)nc(N5CCCn6nc(C(=O)N(C)C)cc6C5)c4c4cn(C)nc34)c12]

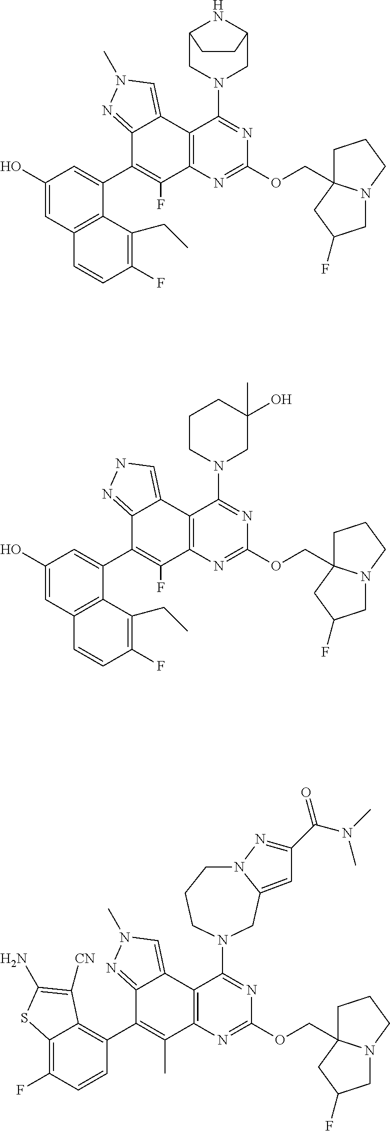 [SMILES: CCc1c(F)ccc2cc(O)cc(-c3c(F)c4nc(OCC56CCCN5CC(F)C6)nc(N5CC6CCC(C5)N6)c4c4cn(C)nc34)c12 ; CCc1c(F)ccc2cc(O)cc(-c3c(F)c4nc(OCC56CCCN5CC(F)C6)nc(N5CCCC(C)(O)C5)c4c4c[nH]nc34)c12 ; Cc1c(-c2ccc(F)c3sc(N)c(C#N)c23)c2nn(C)cc2c2c(N3CCCn4nc(C(=O)N(C)C)cc4C3)nc(OCC34CCCN3CC(F)C4)nc12]

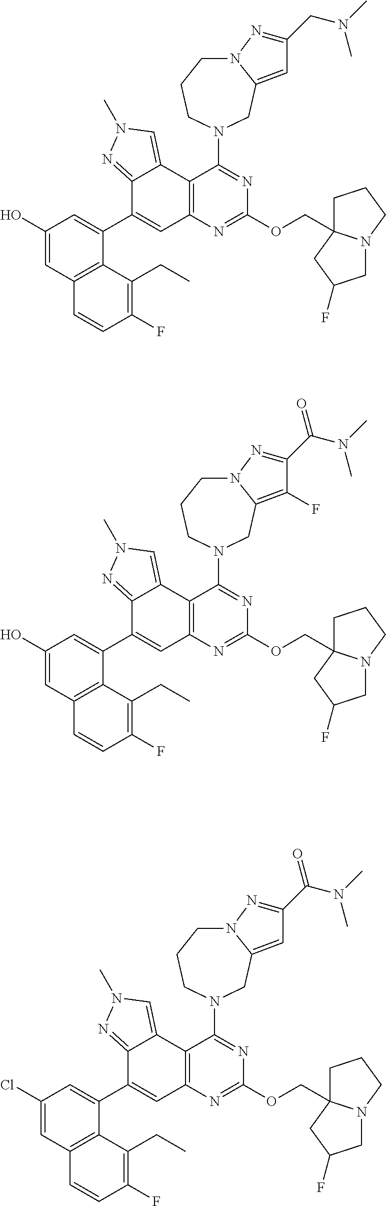 [SMILES: CCc1c(F)ccc2cc(O)cc(-c3cc4nc(OCC56CCCN5CC(F)C6)nc(N5CCCn6nc(CN(C)C)cc6C5)c4c4cn(C)nc34)c12 ; CCc1c(F)ccc2cc(O)cc(-c3cc4nc(OCC56CCCN5CC(F)C6)nc(N5CCCn6nc(C(=O)N(C)C)c(F)c6C5)c4c4cn(C)nc34)c12 ; CCc1c(F)ccc2cc(Cl)cc(-c3cc4nc(OCC56CCCN5CC(F)C6)nc(N5CCCn6nc(C(=O)N(C)C)cc6C5)c4c4cn(C)nc34)c12]

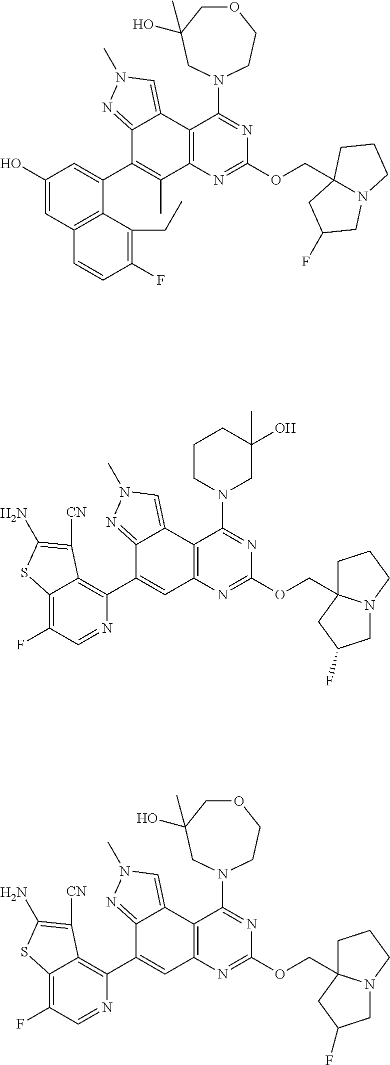 [SMILES: CCc1c(F)ccc2cc(O)cc(-c3c(C)c4nc(OCC56CCCN5CC(F)C6)nc(N5CCOCC(C)(O)C5)c4c4cn(C)nc34)c12 ; Cn1cc2c(n1)c(-c1ncc(F)c3sc(N)c(C#N)c13)cc1nc(OCC34CCCN3C[C@H](F)C4)nc(N3CCCC(C)(O)C3)c12 ; Cn1cc2c(n1)c(-c1ncc(F)c3sc(N)c(C#N)c13)cc1nc(OCC34CCCN3CC(F)C4)nc(N3CCOCC(C)(O)C3)c12]

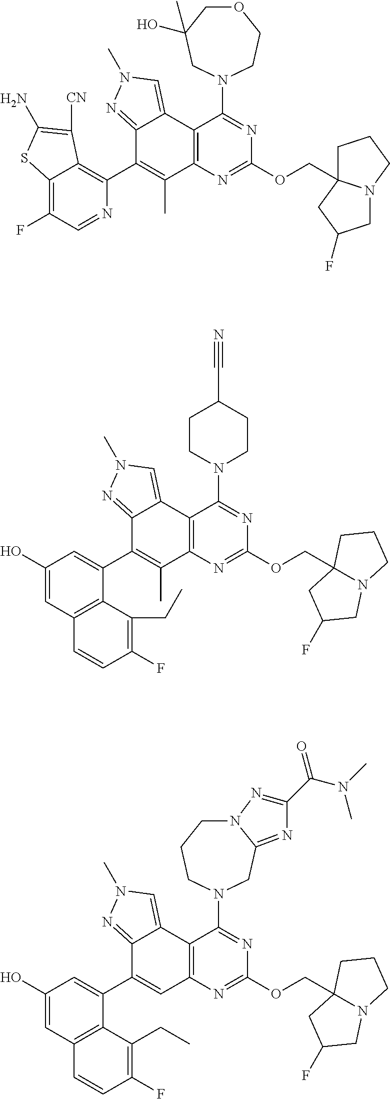 [SMILES: Cc1c(-c2ncc(F)c3sc(N)c(C#N)c23)c2nn(C)cc2c2c(N3CCOCC(C)(O)C3)nc(OCC34CCCN3CC(F)C4)nc12 ; CCc1c(F)ccc2cc(O)cc(-c3c(C)c4nc(OCC56CCCN5CC(F)C6)nc(N5CCC(C#N)CC5)c4c4cn(C)nc34)c12 ; CCc1c(F)ccc2cc(O)cc(-c3cc4nc(OCC56CCCN5CC(F)C6)nc(N5CCCn6nc(C(=O)N(C)C)nc6C5)c4c4cn(C)nc34)c12]

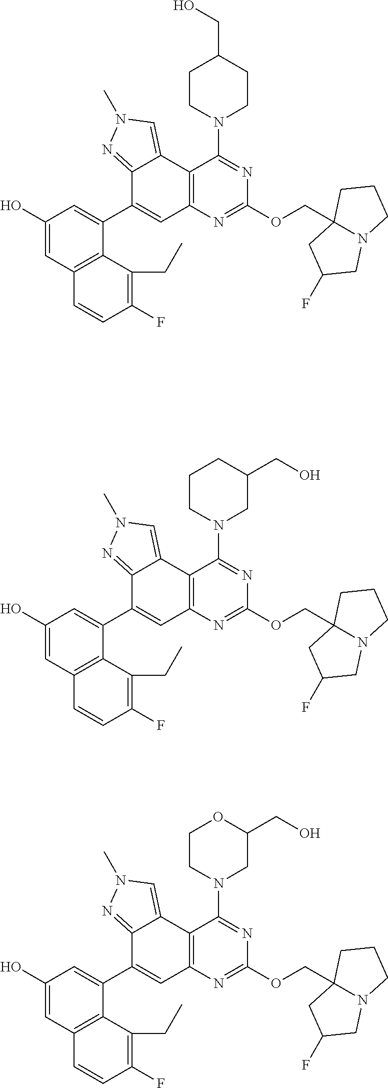 [SMILES: CCc1c(F)ccc2cc(O)cc(-c3cc4nc(OCC56CCCN5CC(F)C6)nc(N5CCC(CO)CC5)c4c4cn(C)nc34)c12 ; CCc1c(F)ccc2cc(O)cc(-c3cc4nc(OCC56CCCN5CC(F)C6)nc(N5CCCC(CO)C5)c4c4cn(C)nc34)c12 ; CCc1c(F)ccc2cc(O)cc(-c3cc4nc(OCC56CCCN5CC(F)C6)nc(N5CCOC(CO)C5)c4c4cn(C)nc34)c12]

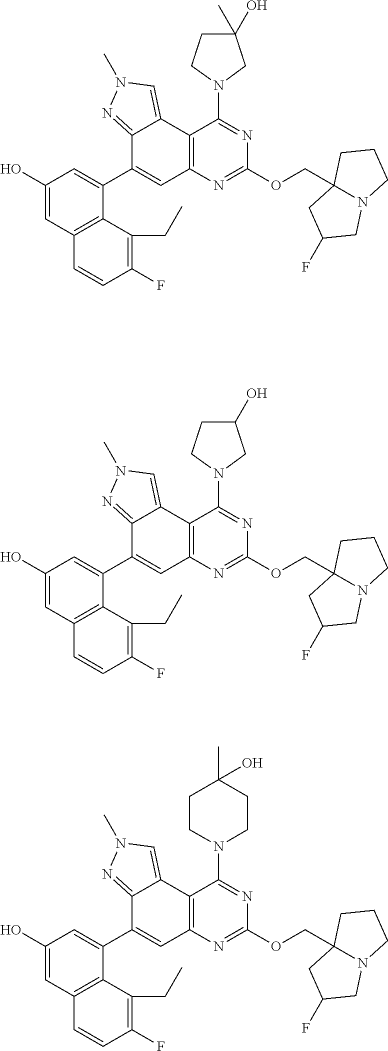 [SMILES: CCc1c(F)ccc2cc(O)cc(-c3cc4nc(OCC56CCCN5CC(F)C6)nc(N5CCC(C)(O)C5)c4c4cn(C)nc34)c12 ; CCc1c(F)ccc2cc(O)cc(-c3cc4nc(OCC56CCCN5CC(F)C6)nc(N5CCC(O)C5)c4c4cn(C)nc34)c12 ; CCc1c(F)ccc2cc(O)cc(-c3cc4nc(OCC56CCCN5CC(F)C6)nc(N5CCC(C)(O)CC5)c4c4cn(C)nc34)c12]

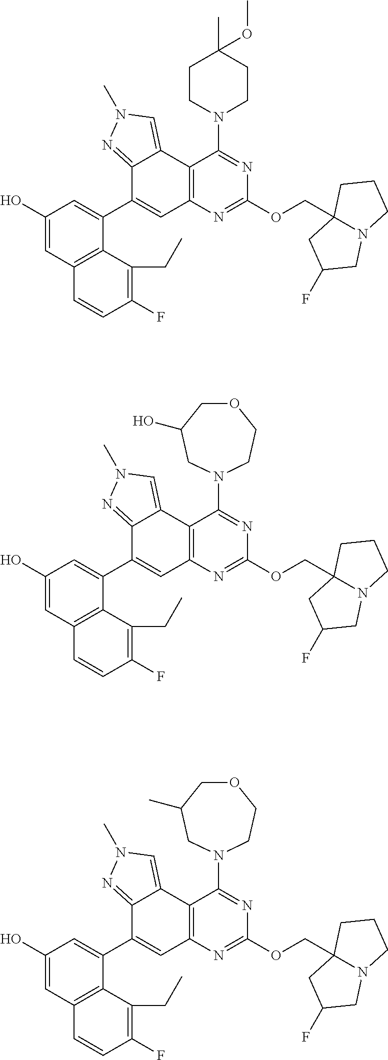 [SMILES: CCc1c(F)ccc2cc(O)cc(-c3cc4nc(OCC56CCCN5CC(F)C6)nc(N5CCC(C)(OC)CC5)c4c4cn(C)nc34)c12 ; CCc1c(F)ccc2cc(O)cc(-c3cc4nc(OCC56CCCN5CC(F)C6)nc(N5CCOCC(O)C5)c4c4cn(C)nc34)c12 ; CCc1c(F)ccc2cc(O)cc(-c3cc4nc(OCC56CCCN5CC(F)C6)nc(N5CCOCC(C)C5)c4c4cn(C)nc34)c12]

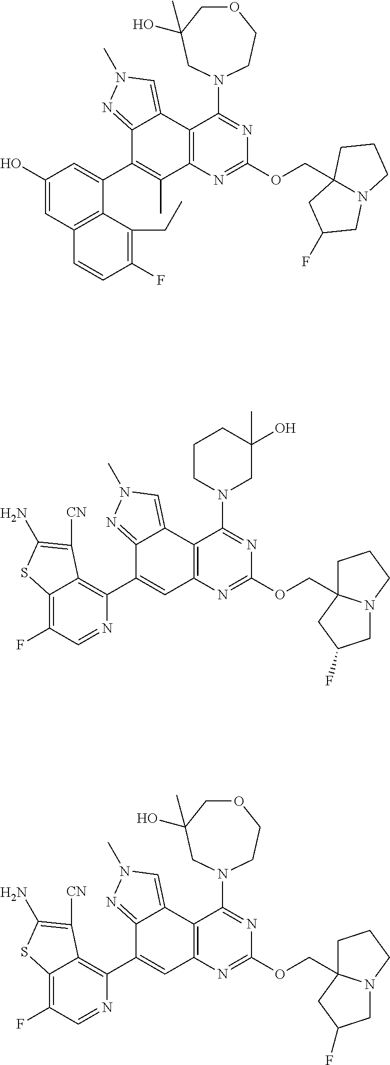 [SMILES: CCc1c(F)ccc2cc(O)cc(-c3c(C)c4nc(OCC56CCCN5CC(F)C6)nc(N5CCOCC(C)(O)C5)c4c4cn(C)nc34)c12 ; Cn1cc2c(n1)c(-c1ncc(F)c3sc(N)c(C#N)c13)cc1nc(OCC34CCCN3C[C@H](F)C4)nc(N3CCCC(C)(O)C3)c12 ; Cn1cc2c(n1)c(-c1ncc(F)c3sc(N)c(C#N)c13)cc1nc(OCC34CCCN3CC(F)C4)nc(N3CCOCC(C)(O)C3)c12]

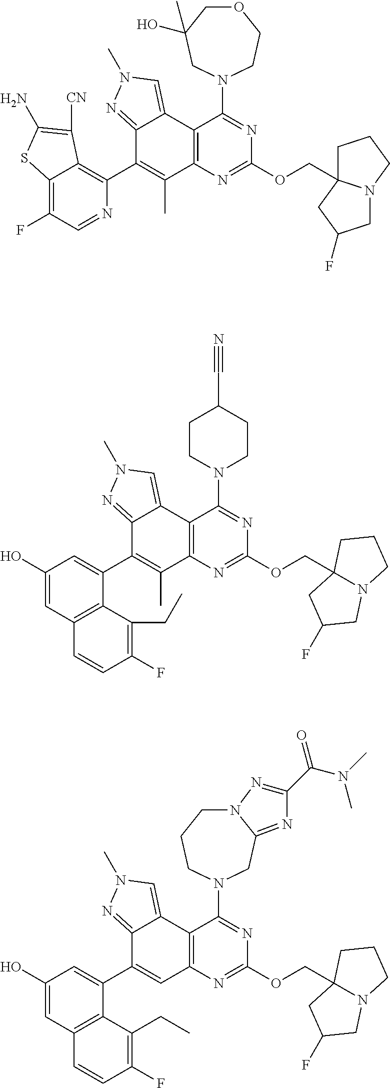 [SMILES: Cc1c(-c2ncc(F)c3sc(N)c(C#N)c23)c2nn(C)cc2c2c(N3CCOCC(C)(O)C3)nc(OCC34CCCN3CC(F)C4)nc12 ; CCc1c(F)ccc2cc(O)cc(-c3c(C)c4nc(OCC56CCCN5CC(F)C6)nc(N5CCC(C#N)CC5)c4c4cn(C)nc34)c12 ; CCc1c(F)ccc2cc(O)cc(-c3cc4nc(OCC56CCCN5CC(F)C6)nc(N5CCCn6nc(C(=O)N(C)C)nc6C5)c4c4cn(C)nc34)c12]

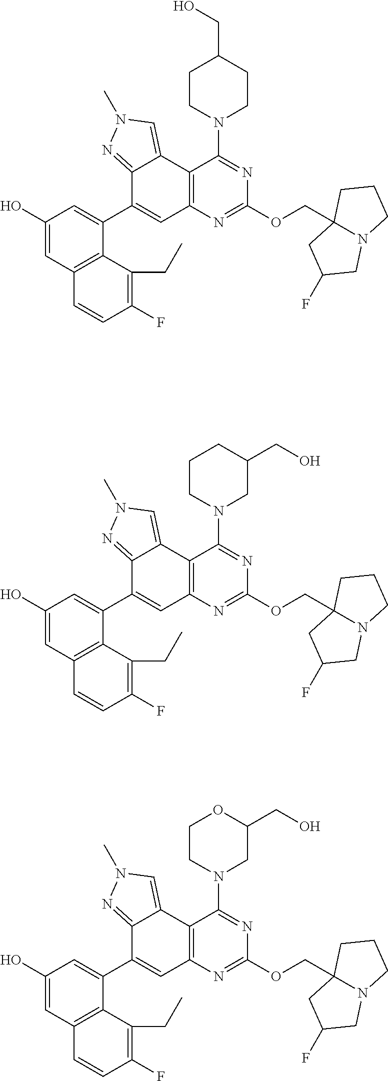 [SMILES: CCc1c(F)ccc2cc(O)cc(-c3cc4nc(OCC56CCCN5CC(F)C6)nc(N5CCC(CO)CC5)c4c4cn(C)nc34)c12 ; CCc1c(F)ccc2cc(O)cc(-c3cc4nc(OCC56CCCN5CC(F)C6)nc(N5CCCC(CO)C5)c4c4cn(C)nc34)c12 ; CCc1c(F)ccc2cc(O)cc(-c3cc4nc(OCC56CCCN5CC(F)C6)nc(N5CCOC(CO)C5)c4c4cn(C)nc34)c12]

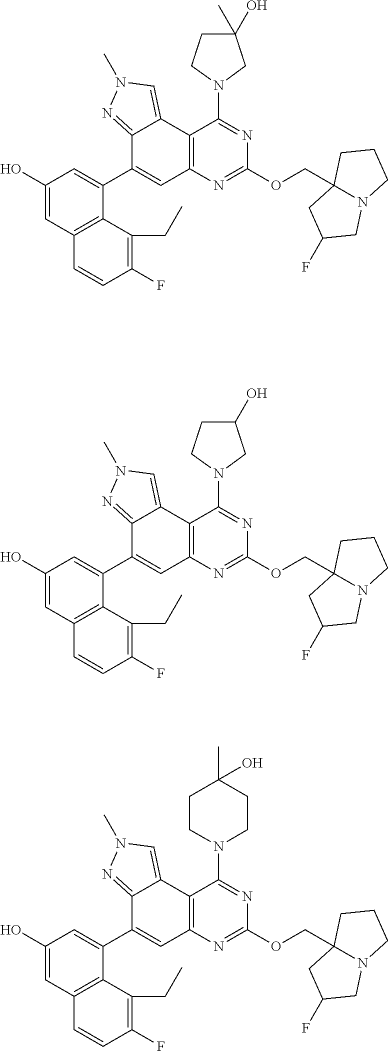 [SMILES: CCc1c(F)ccc2cc(O)cc(-c3cc4nc(OCC56CCCN5CC(F)C6)nc(N5CCC(C)(O)C5)c4c4cn(C)nc34)c12 ; CCc1c(F)ccc2cc(O)cc(-c3cc4nc(OCC56CCCN5CC(F)C6)nc(N5CCC(O)C5)c4c4cn(C)nc34)c12 ; CCc1c(F)ccc2cc(O)cc(-c3cc4nc(OCC56CCCN5CC(F)C6)nc(N5CCC(C)(O)CC5)c4c4cn(C)nc34)c12]

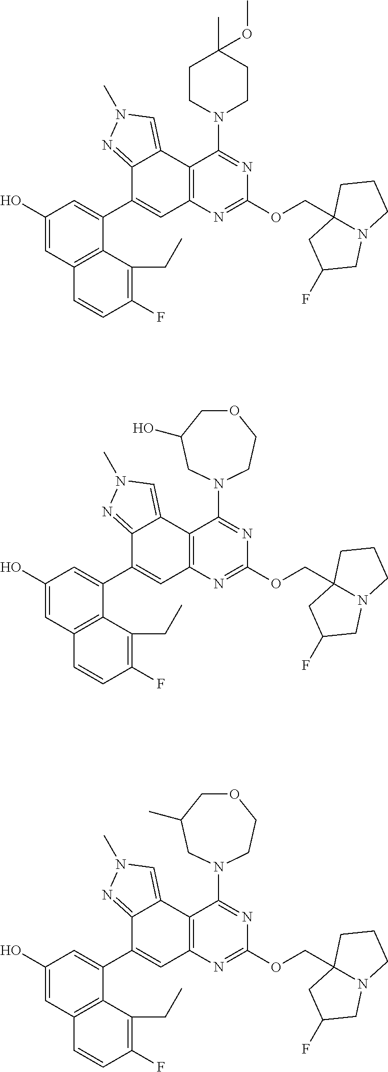 [SMILES: CCc1c(F)ccc2cc(O)cc(-c3cc4nc(OCC56CCCN5CC(F)C6)nc(N5CCC(C)(OC)CC5)c4c4cn(C)nc34)c12 ; CCc1c(F)ccc2cc(O)cc(-c3cc4nc(OCC56CCCN5CC(F)C6)nc(N5CCOCC(O)C5)c4c4cn(C)nc34)c12 ; CCc1c(F)ccc2cc(O)cc(-c3cc4nc(OCC56CCCN5CC(F)C6)nc(N5CCOCC(C)C5)c4c4cn(C)nc34)c12]

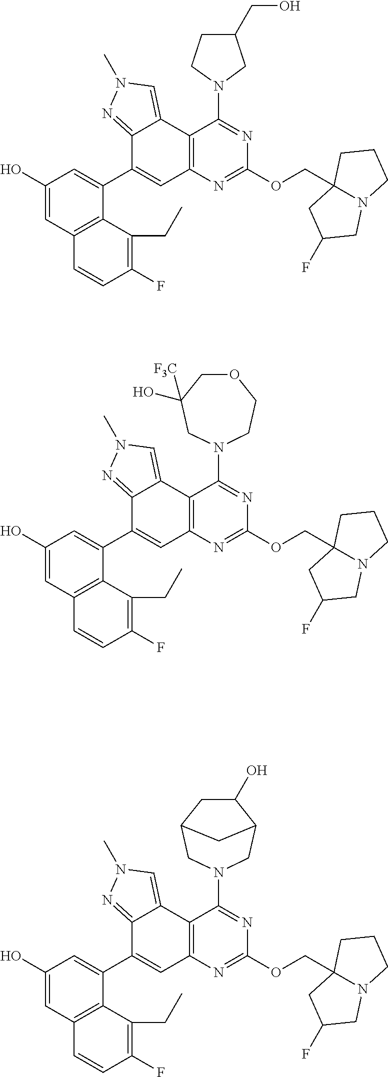 [SMILES: CCc1c(F)ccc2cc(O)cc(-c3cc4nc(OCC56CCCN5CC(F)C6)nc(N5CCC(CO)C5)c4c4cn(C)nc34)c12 ; CCc1c(F)ccc2cc(O)cc(-c3cc4nc(OCC56CCCN5CC(F)C6)nc(N5CCOCC(O)(C(F)(F)F)C5)c4c4cn(C)nc34)c12 ; CCc1c(F)ccc2cc(O)cc(-c3cc4nc(OCC56CCCN5CC(F)C6)nc(N5CC6CC(O)C(C6)C5)c4c4cn(C)nc34)c12]

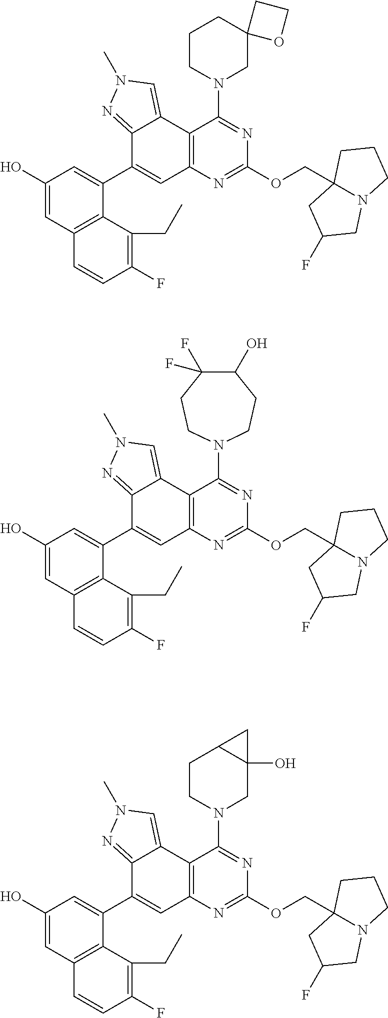 [SMILES: CCc1c(F)ccc2cc(O)cc(-c3cc4nc(OCC56CCCN5CC(F)C6)nc(N5CCCC6(CCO6)C5)c4c4cn(C)nc34)c12 ; CCc1c(F)ccc2cc(O)cc(-c3cc4nc(OCC56CCCN5CC(F)C6)nc(N5CCC(O)C(F)(F)CC5)c4c4cn(C)nc34)c12 ; CCc1c(F)ccc2cc(O)cc(-c3cc4nc(OCC56CCCN5CC(F)C6)nc(N5CCC6CC6(O)C5)c4c4cn(C)nc34)c12]

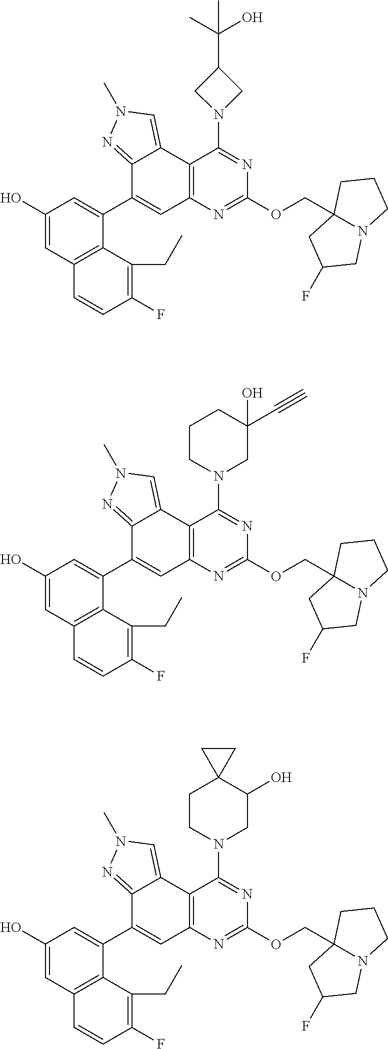 [SMILES: CCc1c(F)ccc2cc(O)cc(-c3cc4nc(OCC56CCCN5CC(F)C6)nc(N5CC(C(C)(C)O)C5)c4c4cn(C)nc34)c12 ; C#CC1(O)CCCN(c2nc(OCC34CCCN3CC(F)C4)nc3cc(-c4cc(O)cc5ccc(F)c(CC)c45)c4nn(C)cc4c23)C1 ; CCc1c(F)ccc2cc(O)cc(-c3cc4nc(OCC56CCCN5CC(F)C6)nc(N5CCC6(CC6)C(O)C5)c4c4cn(C)nc34)c12]

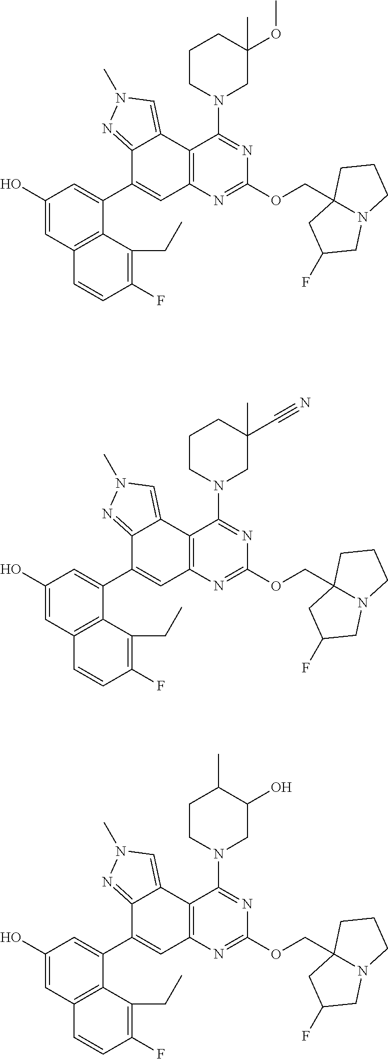 [SMILES: CCc1c(F)ccc2cc(O)cc(-c3cc4nc(OCC56CCCN5CC(F)C6)nc(N5CCCC(C)(OC)C5)c4c4cn(C)nc34)c12 ; CCc1c(F)ccc2cc(O)cc(-c3cc4nc(OCC56CCCN5CC(F)C6)nc(N5CCCC(C)(C#N)C5)c4c4cn(C)nc34)c12 ; CCc1c(F)ccc2cc(O)cc(-c3cc4nc(OCC56CCCN5CC(F)C6)nc(N5CCC(C)C(O)C5)c4c4cn(C)nc34)c12]

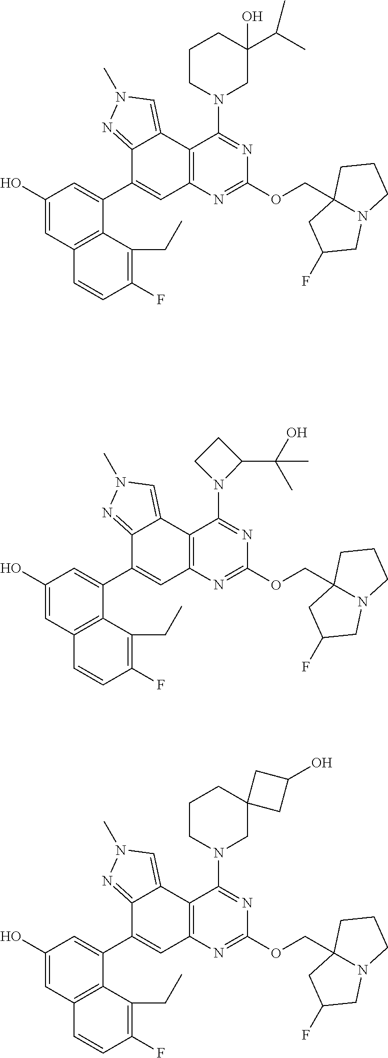 [SMILES: CCc1c(F)ccc2cc(O)cc(-c3cc4nc(OCC56CCCN5CC(F)C6)nc(N5CCCC(O)(C(C)C)C5)c4c4cn(C)nc34)c12 ; CCc1c(F)ccc2cc(O)cc(-c3cc4nc(OCC56CCCN5CC(F)C6)nc(N5CCC5C(C)(C)O)c4c4cn(C)nc34)c12 ; CCc1c(F)ccc2cc(O)cc(-c3cc4nc(OCC56CCCN5CC(F)C6)nc(N5CCCC6(CC(O)C6)C5)c4c4cn(C)nc34)c12]

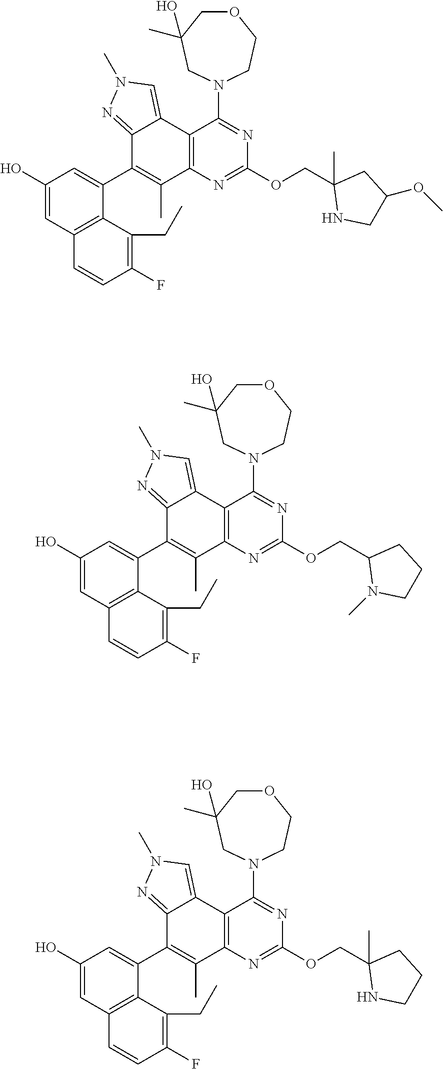 [SMILES: CCc1c(F)ccc2cc(O)cc(-c3c(C)c4nc(OCC5(C)CC(OC)CN5)nc(N5CCOCC(C)(O)C5)c4c4cn(C)nc34)c12 ; CCc1c(F)ccc2cc(O)cc(-c3c(C)c4nc(OCC5CCCN5C)nc(N5CCOCC(C)(O)C5)c4c4cn(C)nc34)c12 ; CCc1c(F)ccc2cc(O)cc(-c3c(C)c4nc(OCC5(C)CCCN5)nc(N5CCOCC(C)(O)C5)c4c4cn(C)nc34)c12]

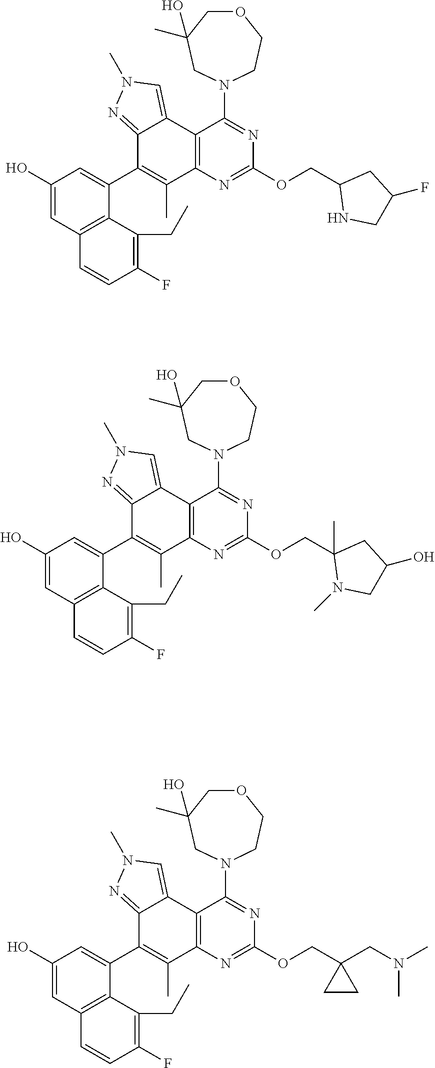 [SMILES: CCc1c(F)ccc2cc(O)cc(-c3c(C)c4nc(OCC5CC(F)CN5)nc(N5CCOCC(C)(O)C5)c4c4cn(C)nc34)c12 ; CCc1c(F)ccc2cc(O)cc(-c3c(C)c4nc(OCC5(C)CC(O)CN5C)nc(N5CCOCC(C)(O)C5)c4c4cn(C)nc34)c12 ; CCc1c(F)ccc2cc(O)cc(-c3c(C)c4nc(OCC5(CN(C)C)CC5)nc(N5CCOCC(C)(O)C5)c4c4cn(C)nc34)c12]

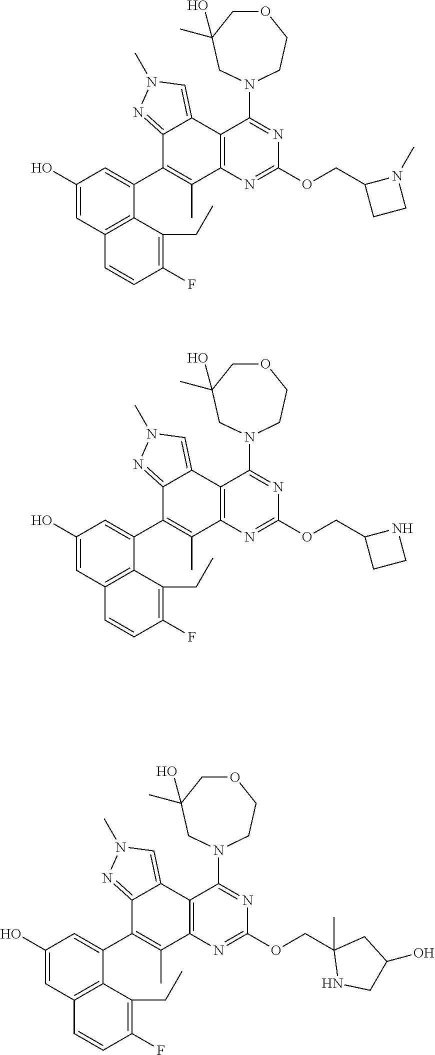 [SMILES: CCc1c(F)ccc2cc(O)cc(-c3c(C)c4nc(OCC5CCN5C)nc(N5CCOCC(C)(O)C5)c4c4cn(C)nc34)c12 ; CCc1c(F)ccc2cc(O)cc(-c3c(C)c4nc(OCC5CCN5)nc(N5CCOCC(C)(O)C5)c4c4cn(C)nc34)c12 ; CCc1c(F)ccc2cc(O)cc(-c3c(C)c4nc(OCC5(C)CC(O)CN5)nc(N5CCOCC(C)(O)C5)c4c4cn(C)nc34)c12]

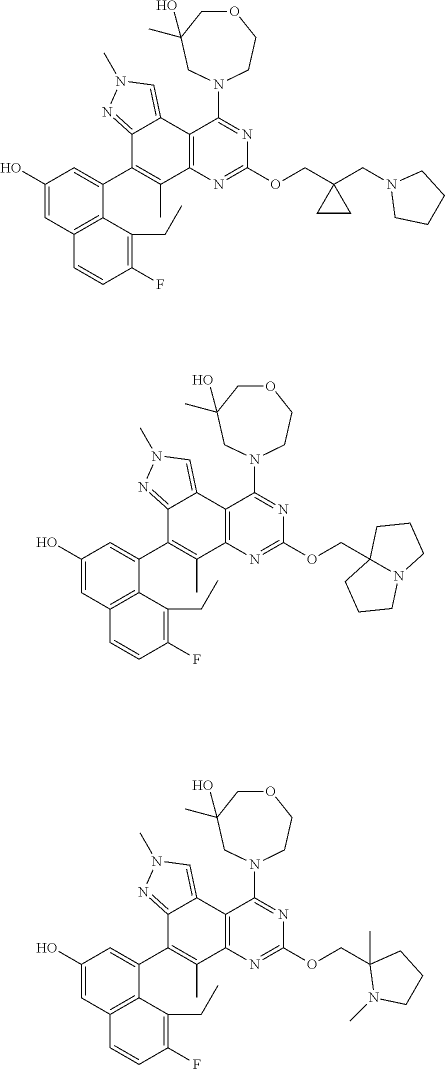 [SMILES: CCc1c(F)ccc2cc(O)cc(-c3c(C)c4nc(OCC5(CN6CCCC6)CC5)nc(N5CCOCC(C)(O)C5)c4c4cn(C)nc34)c12 ; CCc1c(F)ccc2cc(O)cc(-c3c(C)c4nc(OCC56CCCN5CCC6)nc(N5CCOCC(C)(O)C5)c4c4cn(C)nc34)c12 ; CCc1c(F)ccc2cc(O)cc(-c3c(C)c4nc(OCC5(C)CCCN5C)nc(N5CCOCC(C)(O)C5)c4c4cn(C)nc34)c12]

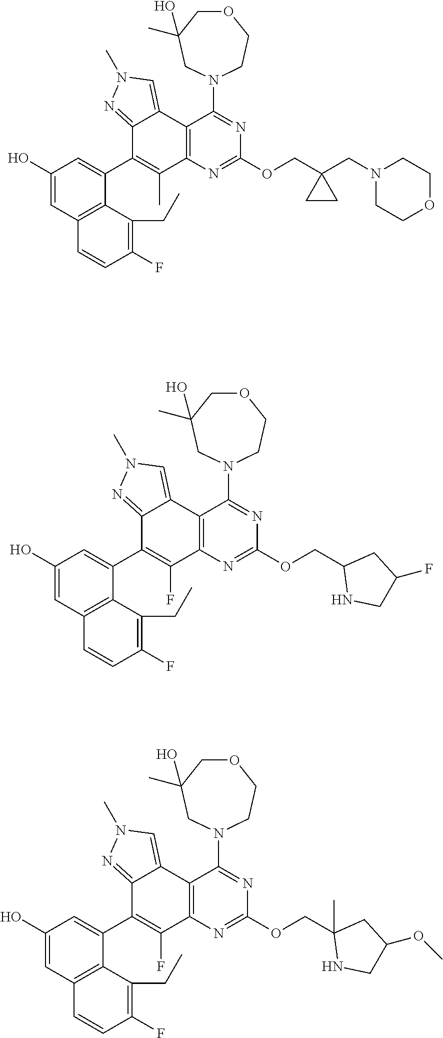 [SMILES: CCc1c(F)ccc2cc(O)cc(-c3c(C)c4nc(OCC5(CN6CCOCC6)CC5)nc(N5CCOCC(C)(O)C5)c4c4cn(C)nc34)c12 ; CCc1c(F)ccc2cc(O)cc(-c3c(F)c4nc(OCC5CC(F)CN5)nc(N5CCOCC(C)(O)C5)c4c4cn(C)nc34)c12 ; CCc1c(F)ccc2cc(O)cc(-c3c(F)c4nc(OCC5(C)CC(OC)CN5)nc(N5CCOCC(C)(O)C5)c4c4cn(C)nc34)c12]

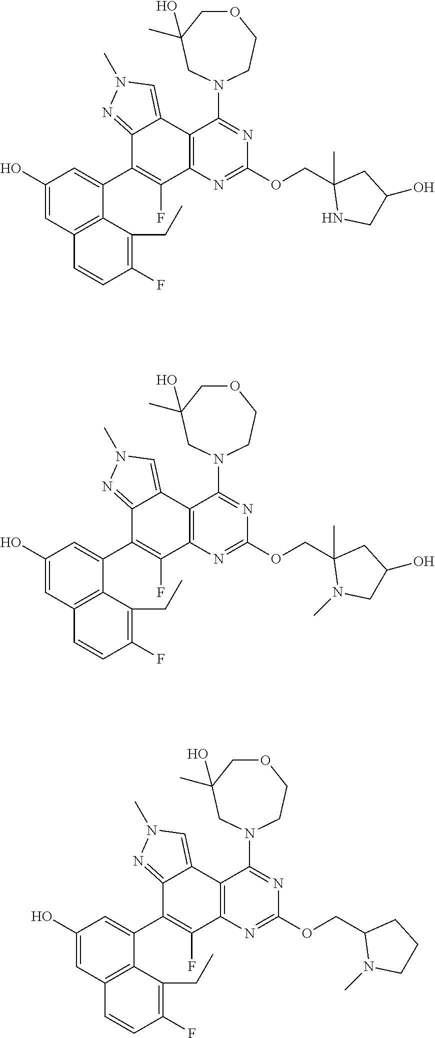 [SMILES: CCc1c(F)ccc2cc(O)cc(-c3c(F)c4nc(OCC5(C)CC(O)CN5)nc(N5CCOCC(C)(O)C5)c4c4cn(C)nc34)c12 ; CCc1c(F)ccc2cc(O)cc(-c3c(F)c4nc(OCC5(C)CC(O)CN5C)nc(N5CCOCC(C)(O)C5)c4c4cn(C)nc34)c12 ; CCc1c(F)ccc2cc(O)cc(-c3c(F)c4nc(OCC5CCCN5C)nc(N5CCOCC(C)(O)C5)c4c4cn(C)nc34)c12]

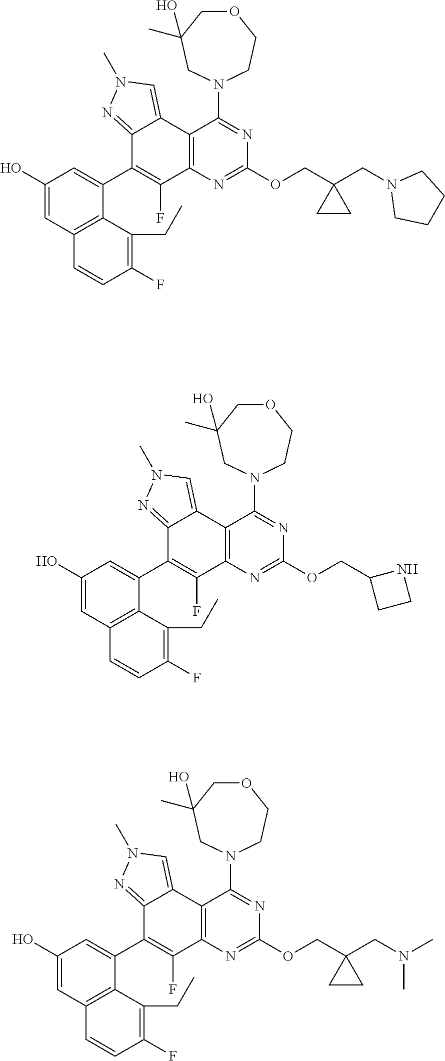 [SMILES: CCc1c(F)ccc2cc(O)cc(-c3c(F)c4nc(OCC5(CN6CCCC6)CC5)nc(N5CCOCC(C)(O)C5)c4c4cn(C)nc34)c12 ; CCc1c(F)ccc2cc(O)cc(-c3c(F)c4nc(OCC5CCN5)nc(N5CCOCC(C)(O)C5)c4c4cn(C)nc34)c12 ; CCc1c(F)ccc2cc(O)cc(-c3c(F)c4nc(OCC5(CN(C)C)CC5)nc(N5CCOCC(C)(O)C5)c4c4cn(C)nc34)c12]

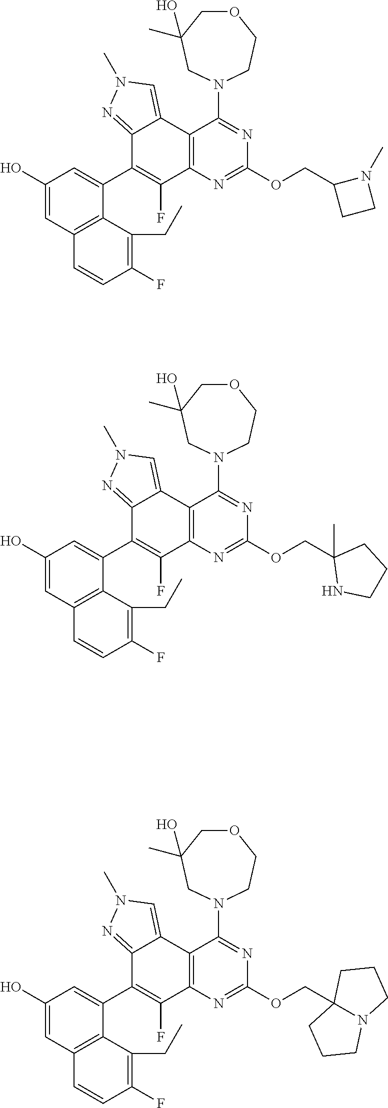 [SMILES: CCc1c(F)ccc2cc(O)cc(-c3c(F)c4nc(OCC5CCN5C)nc(N5CCOCC(C)(O)C5)c4c4cn(C)nc34)c12 ; CCc1c(F)ccc2cc(O)cc(-c3c(F)c4nc(OCC5(C)CCCN5)nc(N5CCOCC(C)(O)C5)c4c4cn(C)nc34)c12 ; CCc1c(F)ccc2cc(O)cc(-c3c(F)c4nc(OCC56CCCN5CCC6)nc(N5CCOCC(C)(O)C5)c4c4cn(C)nc34)c12]

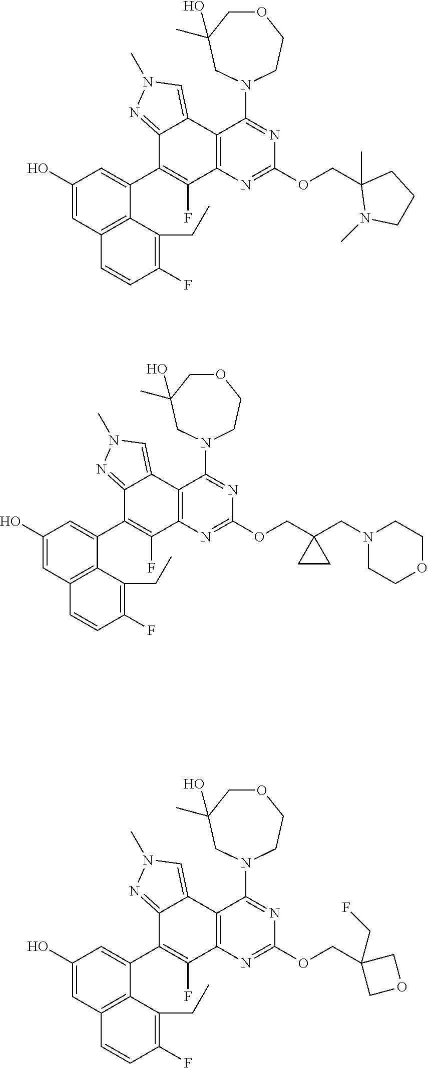 [SMILES: CCc1c(F)ccc2cc(O)cc(-c3c(F)c4nc(OCC5(C)CCCN5C)nc(N5CCOCC(C)(O)C5)c4c4cn(C)nc34)c12 ; CCc1c(F)ccc2cc(O)cc(-c3c(F)c4nc(OCC5(CN6CCOCC6)CC5)nc(N5CCOCC(C)(O)C5)c4c4cn(C)nc34)c12 ; CCc1c(F)ccc2cc(O)cc(-c3c(F)c4nc(OCC5(CF)COC5)nc(N5CCOCC(C)(O)C5)c4c4cn(C)nc34)c12]

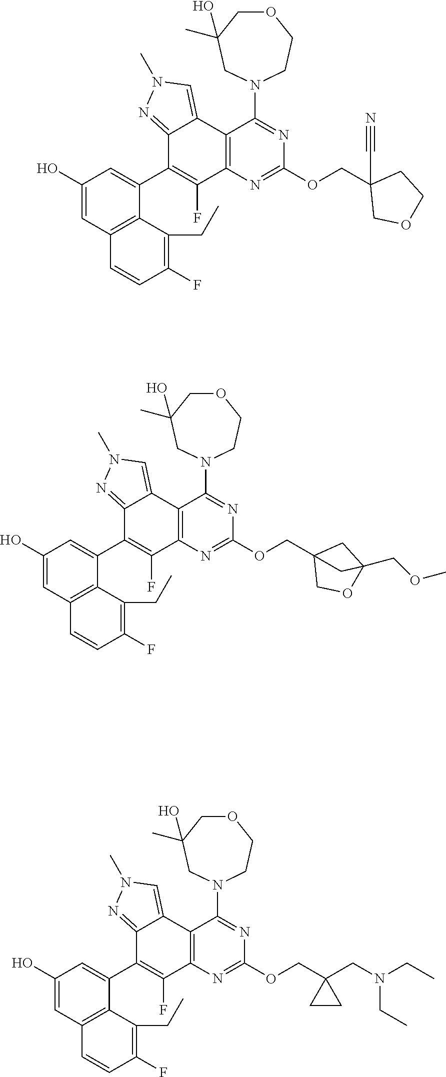 [SMILES: CCc1c(F)ccc2cc(O)cc(-c3c(F)c4nc(OCC5(C#N)CCOC5)nc(N5CCOCC(C)(O)C5)c4c4cn(C)nc34)c12 ; CCc1c(F)ccc2cc(O)cc(-c3c(F)c4nc(OCC56COC(COC)(C5)C6)nc(N5CCOCC(C)(O)C5)c4c4cn(C)nc34)c12 ; CCc1c(F)ccc2cc(O)cc(-c3c(F)c4nc(OCC5(CN(CC)CC)CC5)nc(N5CCOCC(C)(O)C5)c4c4cn(C)nc34)c12]

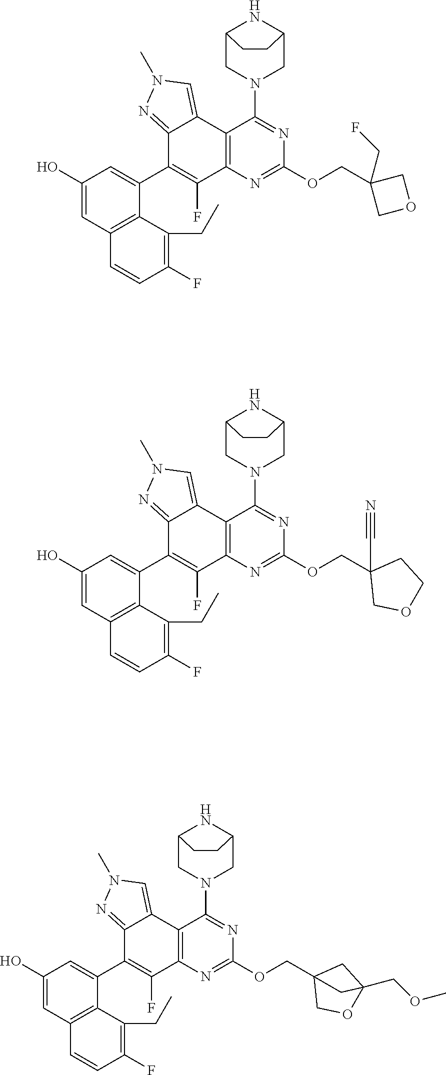 [SMILES: CCc1c(F)ccc2cc(O)cc(-c3c(F)c4nc(OCC5(CF)COC5)nc(N5CC6CCC(C5)N6)c4c4cn(C)nc34)c12 ; CCc1c(F)ccc2cc(O)cc(-c3c(F)c4nc(OCC5(C#N)CCOC5)nc(N5CC6CCC(C5)N6)c4c4cn(C)nc34)c12 ; CCc1c(F)ccc2cc(O)cc(-c3c(F)c4nc(OCC56COC(COC)(C5)C6)nc(N5CC6CCC(C5)N6)c4c4cn(C)nc34)c12]

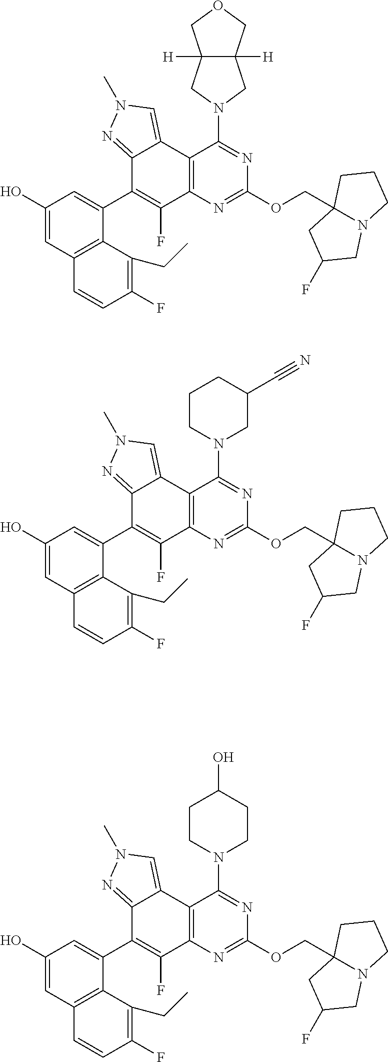 [SMILES: CCc1c(F)ccc2cc(O)cc(-c3c(F)c4nc(OCC56CCCN5CC(F)C6)nc(N5CC6COCC6C5)c4c4cn(C)nc34)c12 ; CCc1c(F)ccc2cc(O)cc(-c3c(F)c4nc(OCC56CCCN5CC(F)C6)nc(N5CCCC(C#N)C5)c4c4cn(C)nc34)c12 ; CCc1c(F)ccc2cc(O)cc(-c3c(F)c4nc(OCC56CCCN5CC(F)C6)nc(N5CCC(O)CC5)c4c4cn(C)nc34)c12]

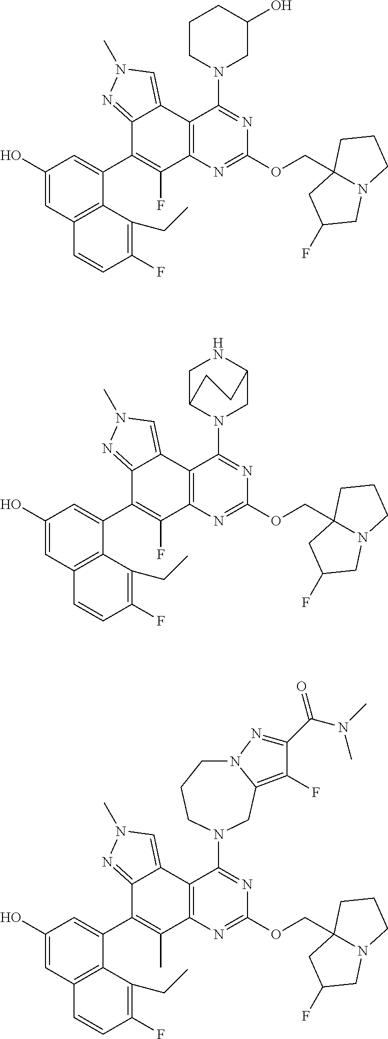 [SMILES: CCc1c(F)ccc2cc(O)cc(-c3c(F)c4nc(OCC56CCCN5CC(F)C6)nc(N5CCCC(O)C5)c4c4cn(C)nc34)c12 ; CCc1c(F)ccc2cc(O)cc(-c3c(F)c4nc(OCC56CCCN5CC(F)C6)nc(N5CC6CCC5CN6)c4c4cn(C)nc34)c12 ; CCc1c(F)ccc2cc(O)cc(-c3c(C)c4nc(OCC56CCCN5CC(F)C6)nc(N5CCCn6nc(C(=O)N(C)C)c(F)c6C5)c4c4cn(C)nc34)c12]

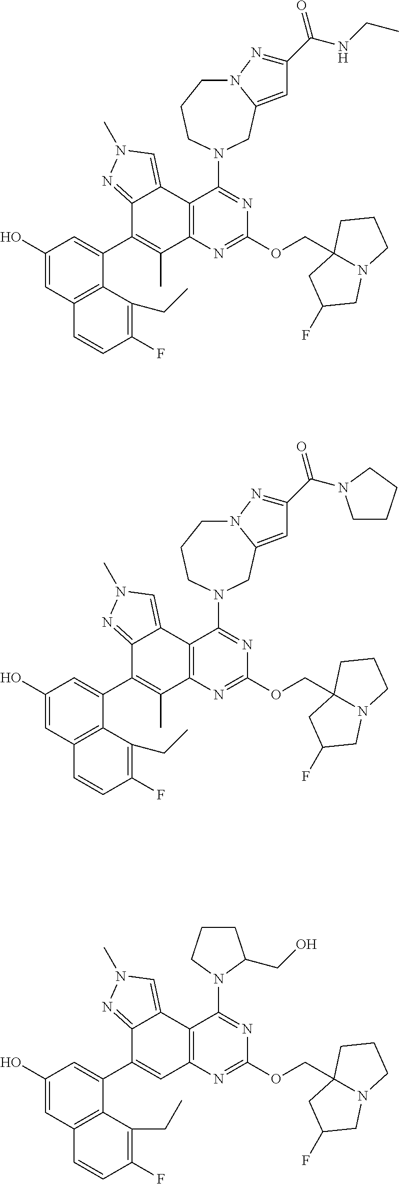 [SMILES: CCNC(=O)c1cc2n(n1)CCCN(c1nc(OCC34CCCN3CC(F)C4)nc3c(C)c(-c4cc(O)cc5ccc(F)c(CC)c45)c4nn(C)cc4c13)C2 ; CCc1c(F)ccc2cc(O)cc(-c3c(C)c4nc(OCC56CCCN5CC(F)C6)nc(N5CCCn6nc(C(=O)N7CCCC7)cc6C5)c4c4cn(C)nc34)c12 ; CCc1c(F)ccc2cc(O)cc(-c3cc4nc(OCC56CCCN5CC(F)C6)nc(N5CCCC5CO)c4c4cn(C)nc34)c12]

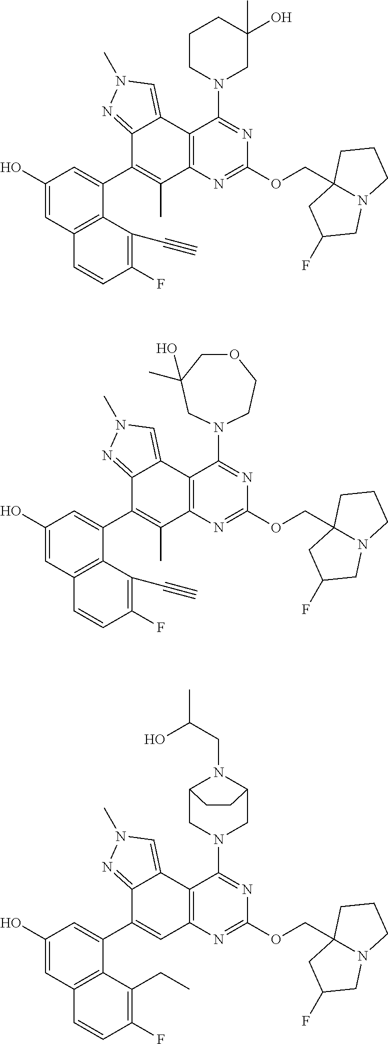 [SMILES: C#Cc1c(F)ccc2cc(O)cc(-c3c(C)c4nc(OCC56CCCN5CC(F)C6)nc(N5CCCC(C)(O)C5)c4c4cn(C)nc34)c12 ; C#Cc1c(F)ccc2cc(O)cc(-c3c(C)c4nc(OCC56CCCN5CC(F)C6)nc(N5CCOCC(C)(O)C5)c4c4cn(C)nc34)c12 ; CCc1c(F)ccc2cc(O)cc(-c3cc4nc(OCC56CCCN5CC(F)C6)nc(N5CC6CCC(C5)N6CC(C)O)c4c4cn(C)nc34)c12]

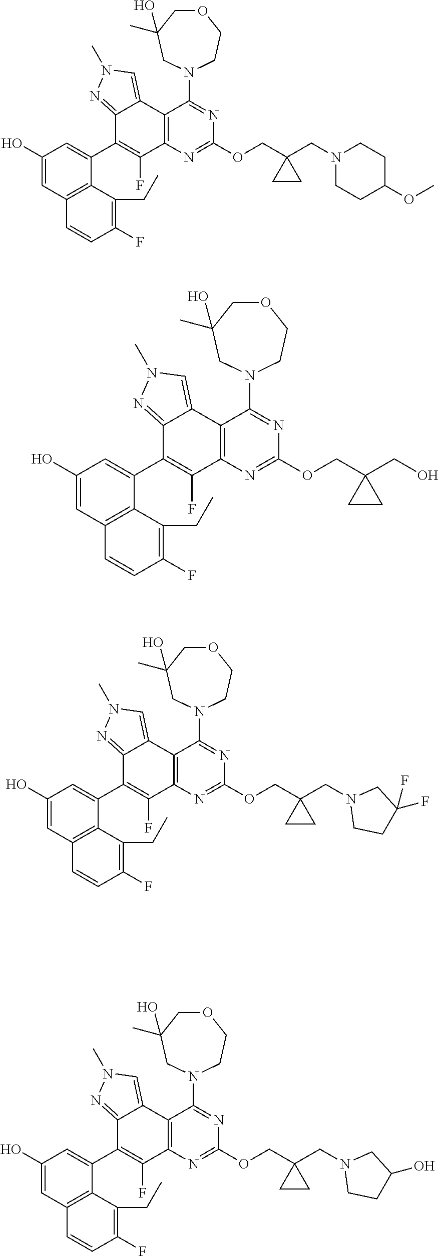 [SMILES: CCc1c(F)ccc2cc(O)cc(-c3c(F)c4nc(OCC5(CN6CCC(OC)CC6)CC5)nc(N5CCOCC(C)(O)C5)c4c4cn(C)nc34)c12 ; CCc1c(F)ccc2cc(O)cc(-c3c(F)c4nc(OCC5(CO)CC5)nc(N5CCOCC(C)(O)C5)c4c4cn(C)nc34)c12 ; CCc1c(F)ccc2cc(O)cc(-c3c(F)c4nc(OCC5(CN6CCC(F)(F)C6)CC5)nc(N5CCOCC(C)(O)C5)c4c4cn(C)nc34)c12 ; CCc1c(F)ccc2cc(O)cc(-c3c(F)c4nc(OCC5(CN6CCC(O)C6)CC5)nc(N5CCOCC(C)(O)C5)c4c4cn(C)nc34)c12]

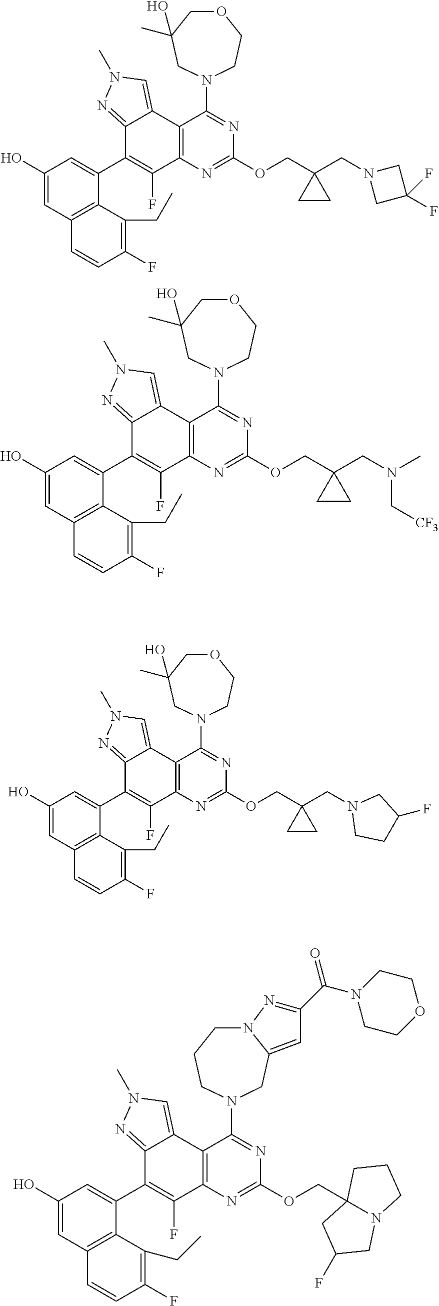

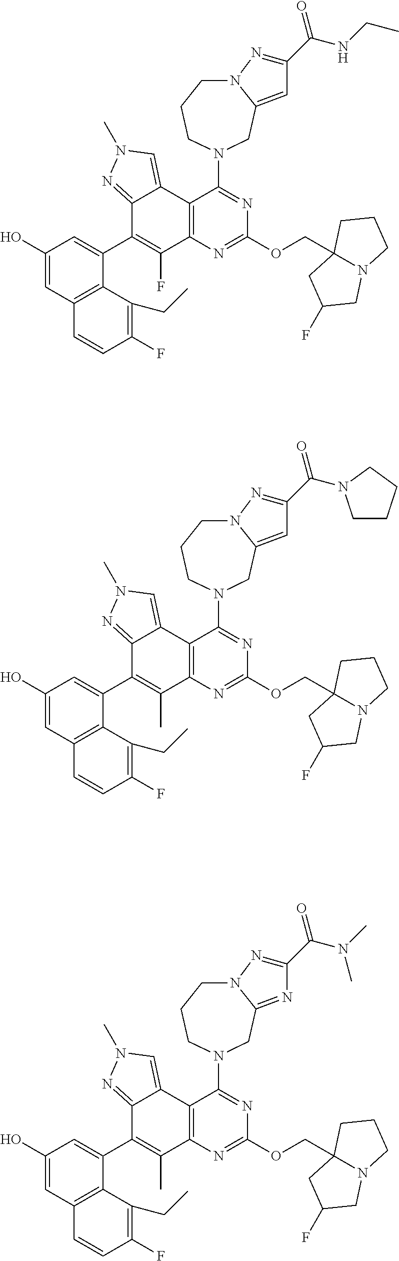 [SMILES: CCNC(=O)c1cc2n(n1)CCCN(c1nc(OCC34CCCN3CC(F)C4)nc3c(F)c(-c4cc(O)cc5ccc(F)c(CC)c45)c4nn(C)cc4c13)C2 ; CCc1c(F)ccc2cc(O)cc(-c3c(C)c4nc(OCC56CCCN5CC(F)C6)nc(N5CCCn6nc(C(=O)N7CCCC7)cc6C5)c4c4cn(C)nc34)c12 ; CCc1c(F)ccc2cc(O)cc(-c3c(C)c4nc(OCC56CCCN5CC(F)C6)nc(N5CCCn6nc(C(=O)N(C)C)nc6C5)c4c4cn(C)nc34)c12]

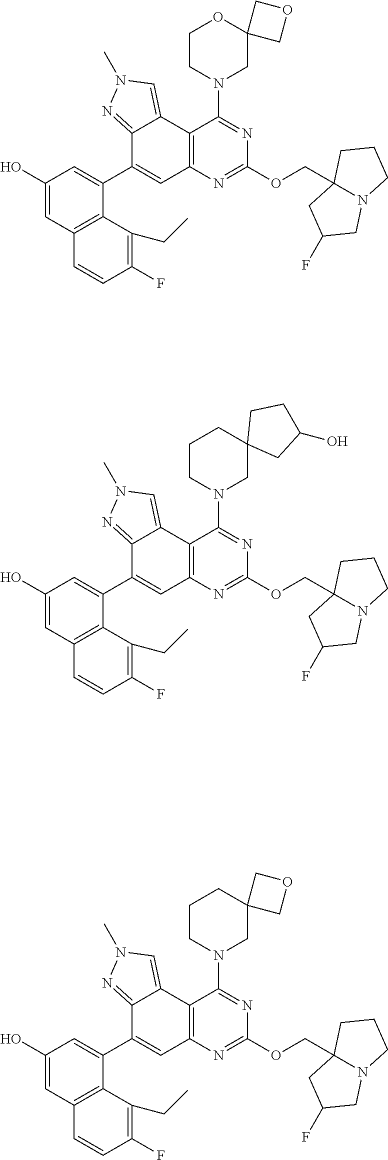 [SMILES: CCc1c(F)ccc2cc(O)cc(-c3cc4nc(OCC56CCCN5CC(F)C6)nc(N5CCOC6(COC6)C5)c4c4cn(C)nc34)c12 ; CCc1c(F)ccc2cc(O)cc(-c3cc4nc(OCC56CCCN5CC(F)C6)nc(N5CCCC6(CCC(O)C6)C5)c4c4cn(C)nc34)c12 ; CCc1c(F)ccc2cc(O)cc(-c3cc4nc(OCC56CCCN5CC(F)C6)nc(N5CCCC6(COC6)C5)c4c4cn(C)nc34)c12]

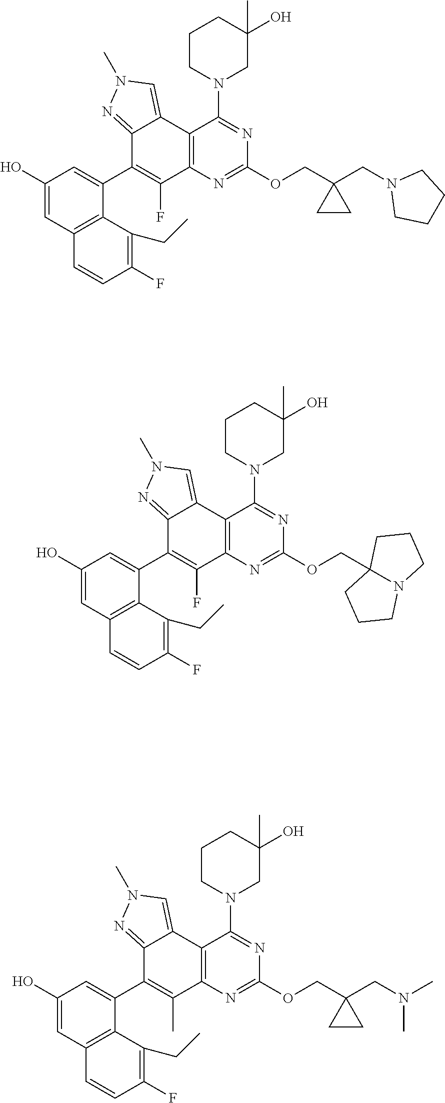 [SMILES: CCc1c(F)ccc2cc(O)cc(-c3c(F)c4nc(OCC5(CN6CCCC6)CC5)nc(N5CCCC(C)(O)C5)c4c4cn(C)nc34)c12 ; CCc1c(F)ccc2cc(O)cc(-c3c(F)c4nc(OCC56CCCN5CCC6)nc(N5CCCC(C)(O)C5)c4c4cn(C)nc34)c12 ; CCc1c(F)ccc2cc(O)cc(-c3c(C)c4nc(OCC5(CN(C)C)CC5)nc(N5CCCC(C)(O)C5)c4c4cn(C)nc34)c12]

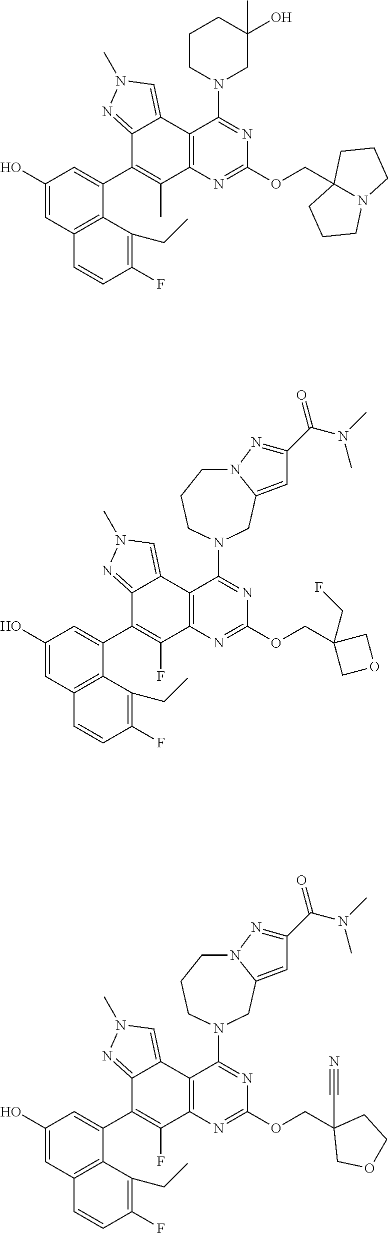 [SMILES: CCc1c(F)ccc2cc(O)cc(-c3c(C)c4nc(OCC56CCCN5CCC6)nc(N5CCCC(C)(O)C5)c4c4cn(C)nc34)c12 ; CCc1c(F)ccc2cc(O)cc(-c3c(F)c4nc(OCC5(CF)COC5)nc(N5CCCn6nc(C(=O)N(C)C)cc6C5)c4c4cn(C)nc34)c12 ; CCc1c(F)ccc2cc(O)cc(-c3c(F)c4nc(OCC5(C#N)CCOC5)nc(N5CCCn6nc(C(=O)N(C)C)cc6C5)c4c4cn(C)nc34)c12]

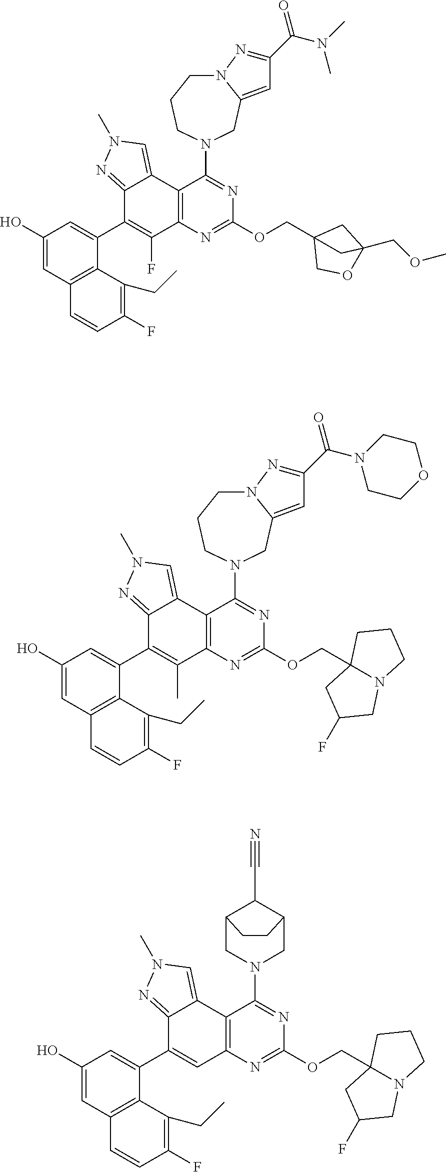 [SMILES: CCc1c(F)ccc2cc(O)cc(-c3c(F)c4nc(OCC56COC(COC)(C5)C6)nc(N5CCCn6nc(C(=O)N(C)C)cc6C5)c4c4cn(C)nc34)c12 ; CCc1c(F)ccc2cc(O)cc(-c3c(C)c4nc(OCC56CCCN5CC(F)C6)nc(N5CCCn6nc(C(=O)N7CCOCC7)cc6C5)c4c4cn(C)nc34)c12 ; CCc1c(F)ccc2cc(O)cc(-c3cc4nc(OCC56CCCN5CC(F)C6)nc(N5CC6CCC(C5)C6C#N)c4c4cn(C)nc34)c12]

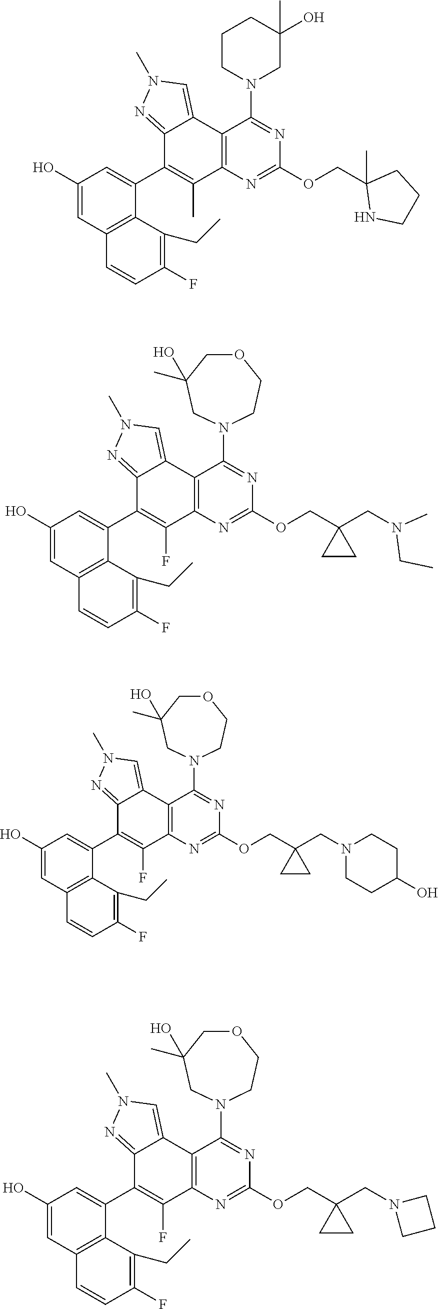 [SMILES: CCc1c(F)ccc2cc(O)cc(-c3c(C)c4nc(OCC5(C)CCCN5)nc(N5CCCC(C)(O)C5)c4c4cn(C)nc34)c12 ; CCc1c(F)ccc2cc(O)cc(-c3c(F)c4nc(OCC5(CN(C)CC)CC5)nc(N5CCOCC(C)(O)C5)c4c4cn(C)nc34)c12 ; CCc1c(F)ccc2cc(O)cc(-c3c(F)c4nc(OCC5(CN6CCC(O)CC6)CC5)nc(N5CCOCC(C)(O)C5)c4c4cn(C)nc34)c12 ; CCc1c(F)ccc2cc(O)cc(-c3c(F)c4nc(OCC5(CN6CCC6)CC5)nc(N5CCOCC(C)(O)C5)c4c4cn(C)nc34)c12]

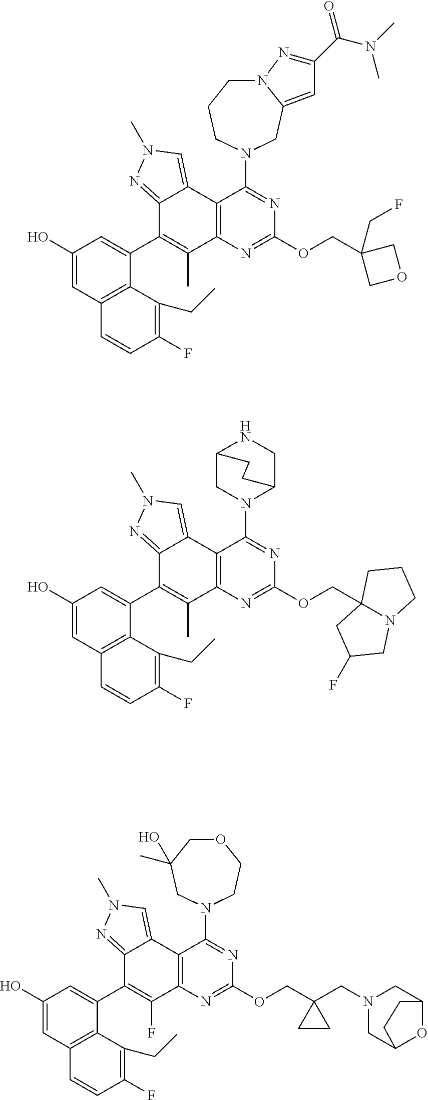 [SMILES: CCc1c(F)ccc2cc(O)cc(-c3c(C)c4nc(OCC5(CF)COC5)nc(N5CCCn6nc(C(=O)N(C)C)cc6C5)c4c4cn(C)nc34)c12 ; CCc1c(F)ccc2cc(O)cc(-c3c(C)c4nc(OCC56CCCN5CC(F)C6)nc(N5CC6CCC5CN6)c4c4cn(C)nc34)c12 ; CCc1c(F)ccc2cc(O)cc(-c3c(F)c4nc(OCC5(CN6CC7CCC(C6)O7)CC5)nc(N5CCOCC(C)(O)C5)c4c4cn(C)nc34)c12]

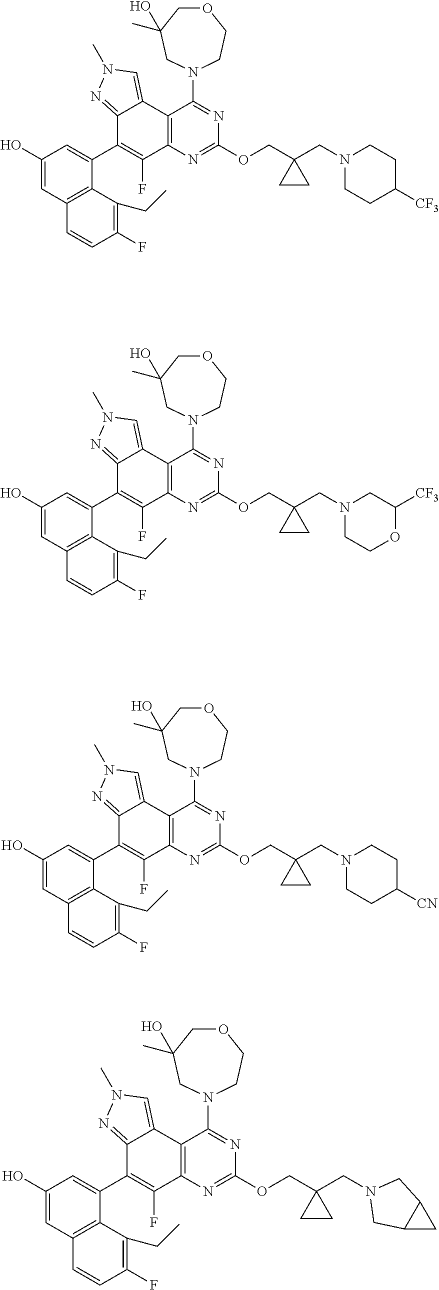 [SMILES: CCc1c(F)ccc2cc(O)cc(-c3c(F)c4nc(OCC5(CN6CCC(C(F)(F)F)CC6)CC5)nc(N5CCOCC(C)(O)C5)c4c4cn(C)nc34)c12 ; CCc1c(F)ccc2cc(O)cc(-c3c(F)c4nc(OCC5(CN6CCOC(C(F)(F)F)C6)CC5)nc(N5CCOCC(C)(O)C5)c4c4cn(C)nc34)c12 ; CCc1c(F)ccc2cc(O)cc(-c3c(F)c4nc(OCC5(CN6CCC(C#N)CC6)CC5)nc(N5CCOCC(C)(O)C5)c4c4cn(C)nc34)c12 ; CCc1c(F)ccc2cc(O)cc(-c3c(F)c4nc(OCC5(CN6CC7CC7C6)CC5)nc(N5CCOCC(C)(O)C5)c4c4cn(C)nc34)c12]

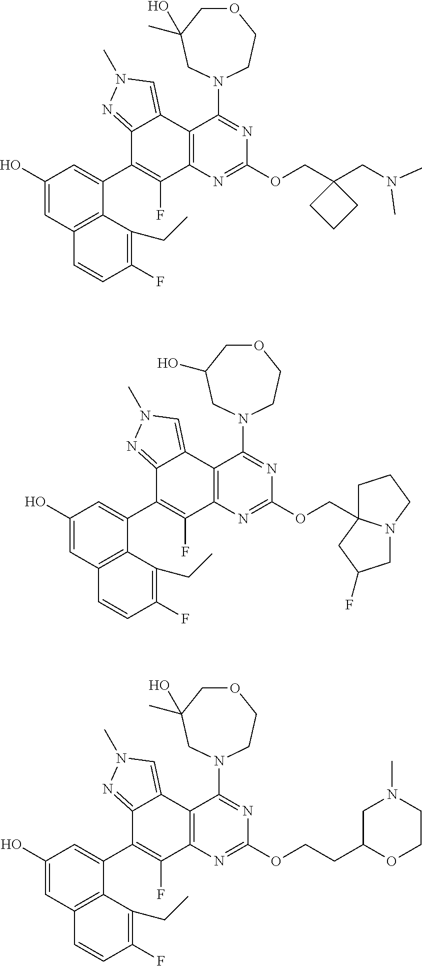 [SMILES: CCc1c(F)ccc2cc(O)cc(-c3c(F)c4nc(OCC5(CN(C)C)CCC5)nc(N5CCOCC(C)(O)C5)c4c4cn(C)nc34)c12 ; CCc1c(F)ccc2cc(O)cc(-c3c(F)c4nc(OCC56CCCN5CC(F)C6)nc(N5CCOCC(O)C5)c4c4cn(C)nc34)c12 ; CCc1c(F)ccc2cc(O)cc(-c3c(F)c4nc(OCCC5CN(C)CCO5)nc(N5CCOCC(C)(O)C5)c4c4cn(C)nc34)c12]

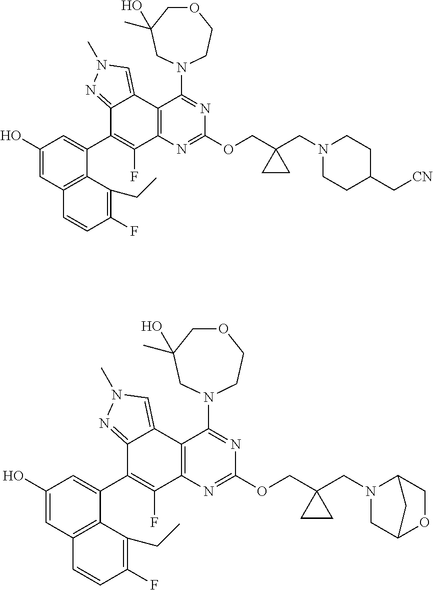 [SMILES: CCc1c(F)ccc2cc(O)cc(-c3c(F)c4nc(OCC5(CN6CCC(CC#N)CC6)CC5)nc(N5CCOCC(C)(O)C5)c4c4cn(C)nc34)c12 ; CCc1c(F)ccc2cc(O)cc(-c3c(F)c4nc(OCC5(CN6CC7CC6CO7)CC5)nc(N5CCOCC(C)(O)C5)c4c4cn(C)nc34)c12]

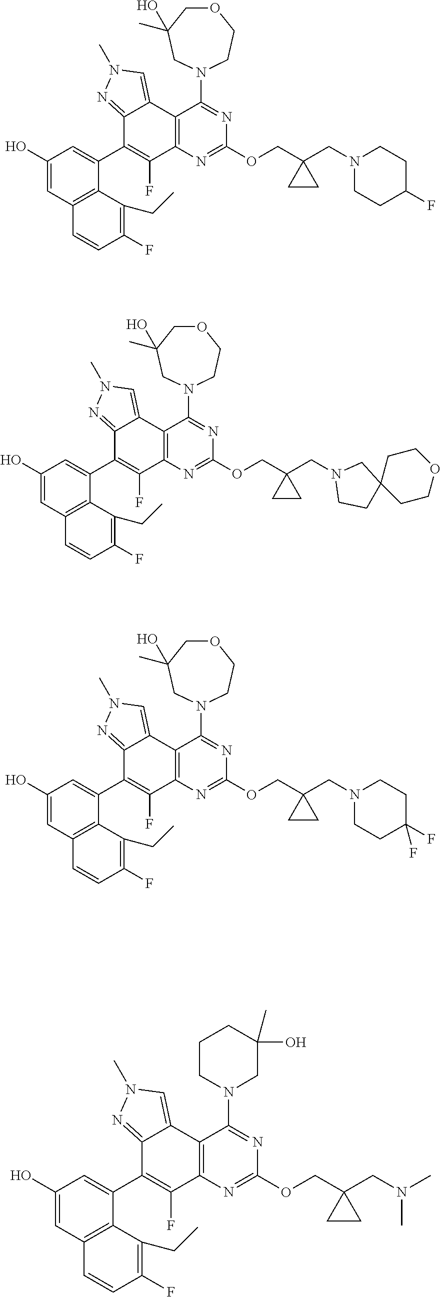 [SMILES: CCc1c(F)ccc2cc(O)cc(-c3c(F)c4nc(OCC5(CN6CCC(F)CC6)CC5)nc(N5CCOCC(C)(O)C5)c4c4cn(C)nc34)c12 ; CCc1c(F)ccc2cc(O)cc(-c3c(F)c4nc(OCC5(CN6CCC7(CCOCC7)C6)CC5)nc(N5CCOCC(C)(O)C5)c4c4cn(C)nc34)c12 ; CCc1c(F)ccc2cc(O)cc(-c3c(F)c4nc(OCC5(CN6CCC(F)(F)CC6)CC5)nc(N5CCOCC(C)(O)C5)c4c4cn(C)nc34)c12 ; CCc1c(F)ccc2cc(O)cc(-c3c(F)c4nc(OCC5(CN(C)C)CC5)nc(N5CCCC(C)(O)C5)c4c4cn(C)nc34)c12]

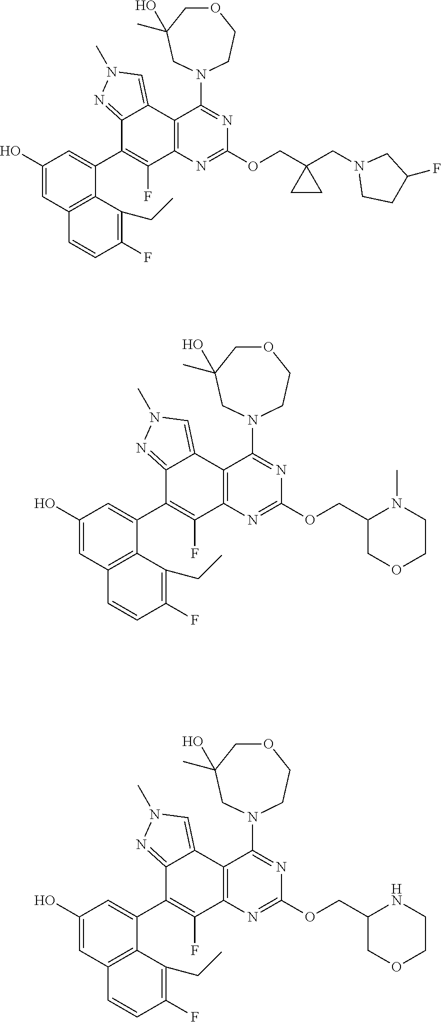 [SMILES: CCc1c(F)ccc2cc(O)cc(-c3c(F)c4nc(OCC5(CN6CCC(F)C6)CC5)nc(N5CCOCC(C)(O)C5)c4c4cn(C)nc34)c12 ; CCc1c(F)ccc2cc(O)cc(-c3c(F)c4nc(OCC5COCCN5C)nc(N5CCOCC(C)(O)C5)c4c4cn(C)nc34)c12 ; CCc1c(F)ccc2cc(O)cc(-c3c(F)c4nc(OCC5COCCN5)nc(N5CCOCC(C)(O)C5)c4c4cn(C)nc34)c12]

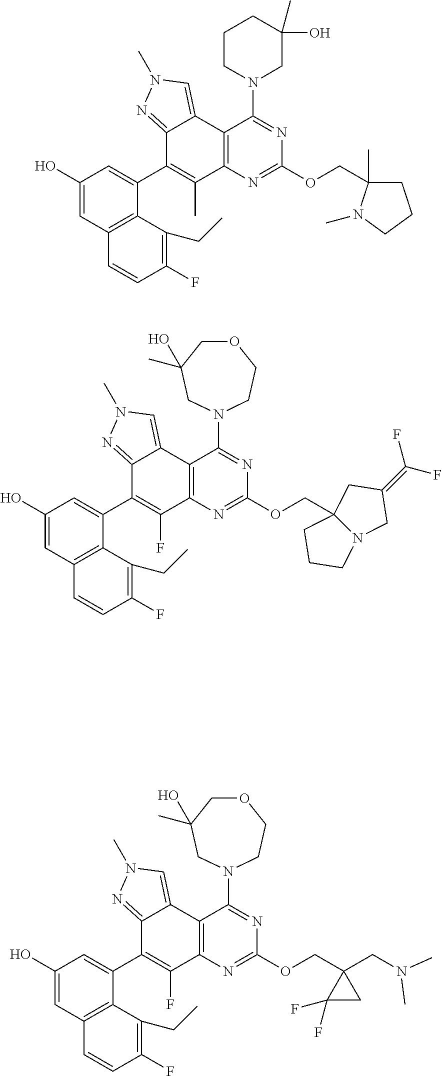 [SMILES: CCc1c(F)ccc2cc(O)cc(-c3c(C)c4nc(OCC5(C)CCCN5C)nc(N5CCCC(C)(O)C5)c4c4cn(C)nc34)c12 ; CCc1c(F)ccc2cc(O)cc(-c3c(F)c4nc(OCC56CCCN5CC(=C(F)F)C6)nc(N5CCOCC(C)(O)C5)c4c4cn(C)nc34)c12 ; CCc1c(F)ccc2cc(O)cc(-c3c(F)c4nc(OCC5(CN(C)C)CC5(F)F)nc(N5CCOCC(C)(O)C5)c4c4cn(C)nc34)c12]

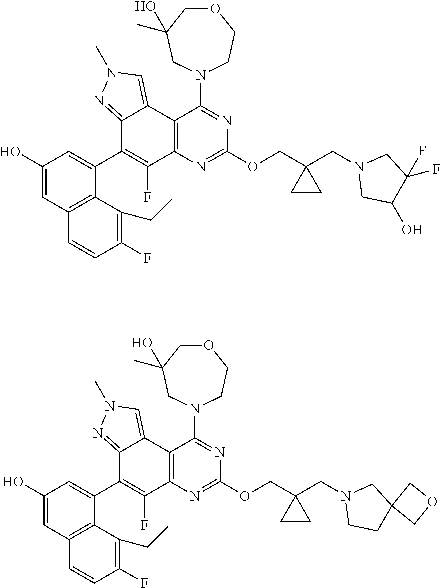 [SMILES: CCc1c(F)ccc2cc(O)cc(-c3c(F)c4nc(OCC5(CN6CC(O)C(F)(F)C6)CC5)nc(N5CCOCC(C)(O)C5)c4c4cn(C)nc34)c12 ; CCc1c(F)ccc2cc(O)cc(-c3c(F)c4nc(OCC5(CN6CCC7(COC7)C6)CC5)nc(N5CCOCC(C)(O)C5)c4c4cn(C)nc34)c12]

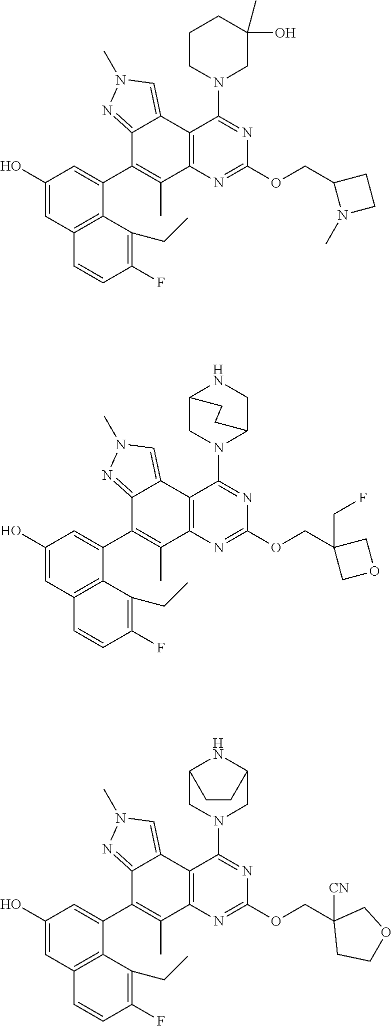 [SMILES: CCc1c(F)ccc2cc(O)cc(-c3c(C)c4nc(OCC5CCN5C)nc(N5CCCC(C)(O)C5)c4c4cn(C)nc34)c12 ; CCc1c(F)ccc2cc(O)cc(-c3c(C)c4nc(OCC5(CF)COC5)nc(N5CC6CCC5CN6)c4c4cn(C)nc34)c12 ; CCc1c(F)ccc2cc(O)cc(-c3c(C)c4nc(OCC5(C#N)CCOC5)nc(N5CC6CCC(C5)N6)c4c4cn(C)nc34)c12]

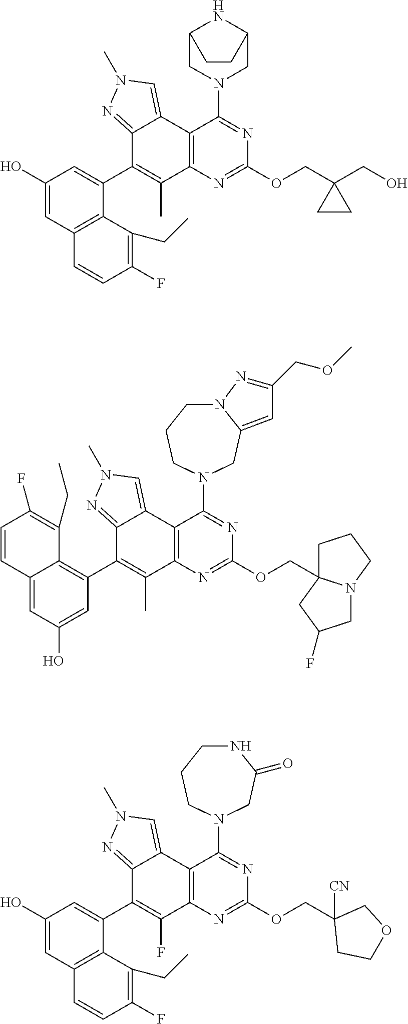 [SMILES: CCc1c(F)ccc2cc(O)cc(-c3c(C)c4nc(OCC5(CO)CC5)nc(N5CC6CCC(C5)N6)c4c4cn(C)nc34)c12 ; CCc1c(F)ccc2cc(O)cc(-c3c(C)c4nc(OCC56CCCN5CC(F)C6)nc(N5CCCn6nc(COC)cc6C5)c4c4cn(C)nc34)c12 ; CCc1c(F)ccc2cc(O)cc(-c3c(F)c4nc(OCC5(C#N)CCOC5)nc(N5CCCNC(=O)C5)c4c4cn(C)nc34)c12]

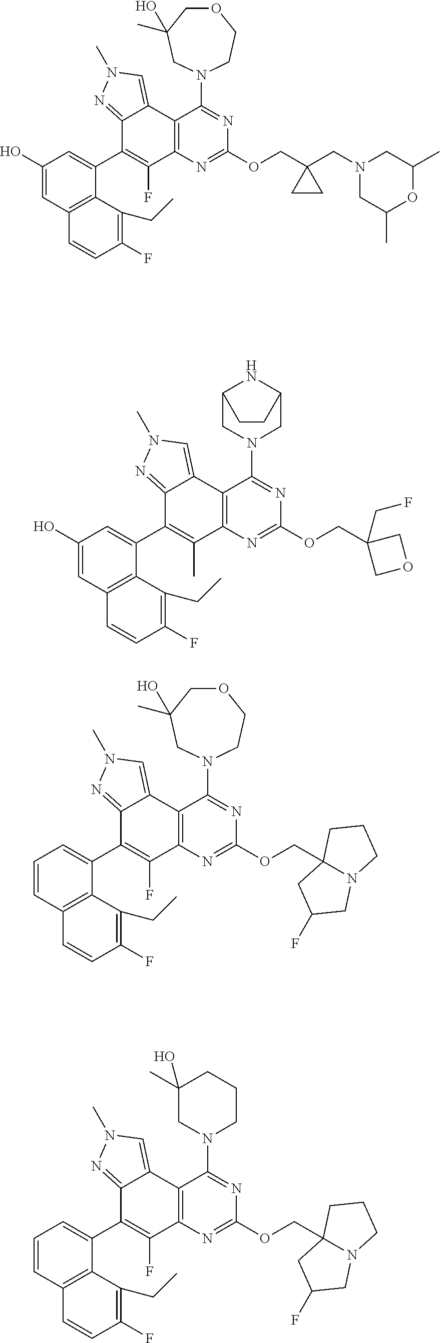

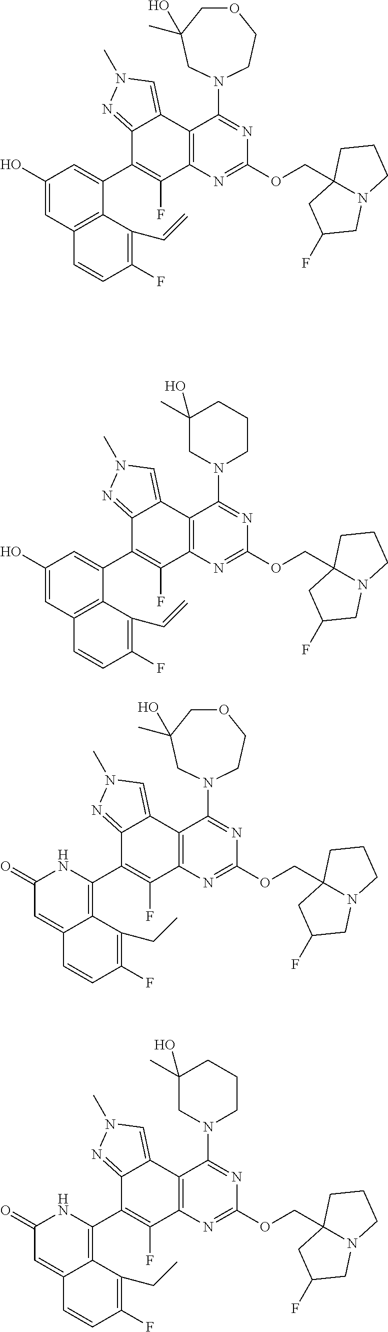

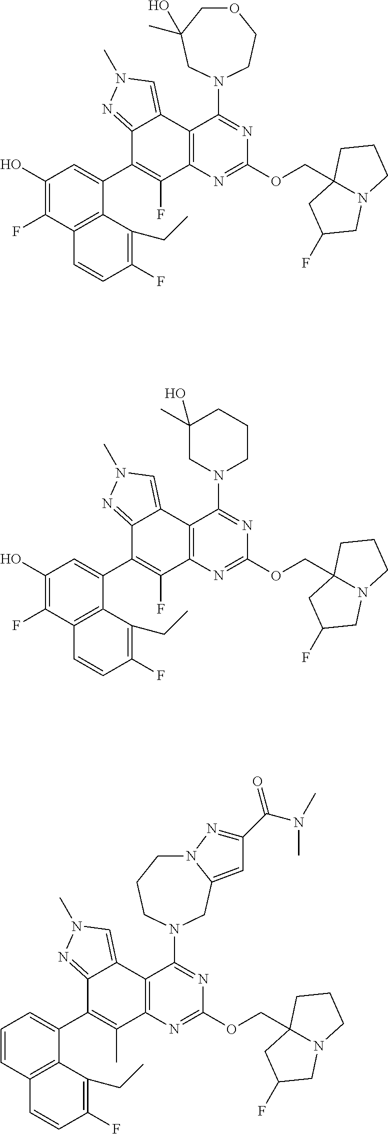 [SMILES: CCc1c(F)ccc2c(F)c(O)cc(-c3c(F)c4nc(OCC56CCCN5CC(F)C6)nc(N5CCOCC(C)(O)C5)c4c4cn(C)nc34)c12 ; CCc1c(F)ccc2c(F)c(O)cc(-c3c(F)c4nc(OCC56CCCN5CC(F)C6)nc(N5CCCC(C)(O)C5)c4c4cn(C)nc34)c12 ; CCc1c(F)ccc2cccc(-c3c(C)c4nc(OCC56CCCN5CC(F)C6)nc(N5CCCn6nc(C(=O)N(C)C)cc6C5)c4c4cn(C)nc34)c12]

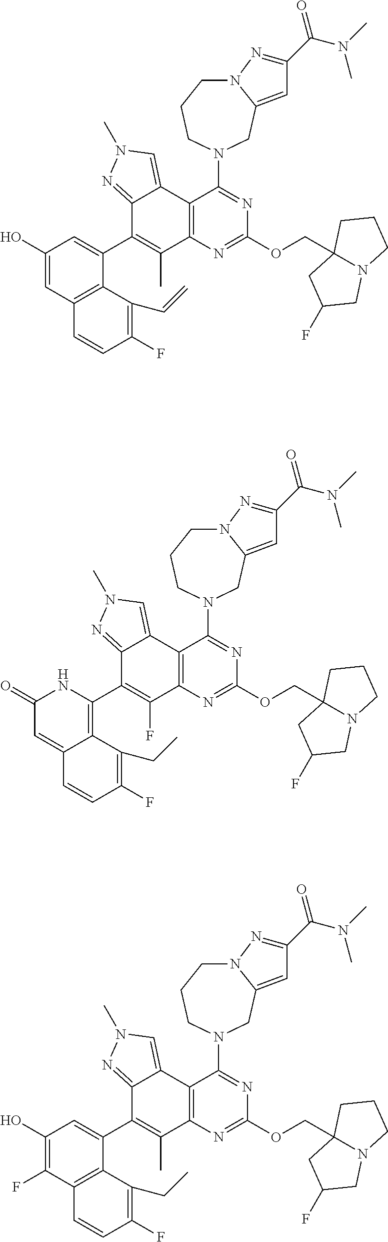 [SMILES: C=Cc1c(F)ccc2cc(O)cc(-c3c(C)c4nc(OCC56CCCN5CC(F)C6)nc(N5CCCn6nc(C(=O)N(C)C)cc6C5)c4c4cn(C)nc34)c12 ; CCc1c(F)ccc2cc(=O)[nH]c(-c3c(F)c4nc(OCC56CCCN5CC(F)C6)nc(N5CCCn6nc(C(=O)N(C)C)cc6C5)c4c4cn(C)nc34)c12 ; CCc1c(F)ccc2c(F)c(O)cc(-c3c(C)c4nc(OCC56CCCN5CC(F)C6)nc(N5CCCn6nc(C(=O)N(C)C)cc6C5)c4c4cn(C)nc34)c12]

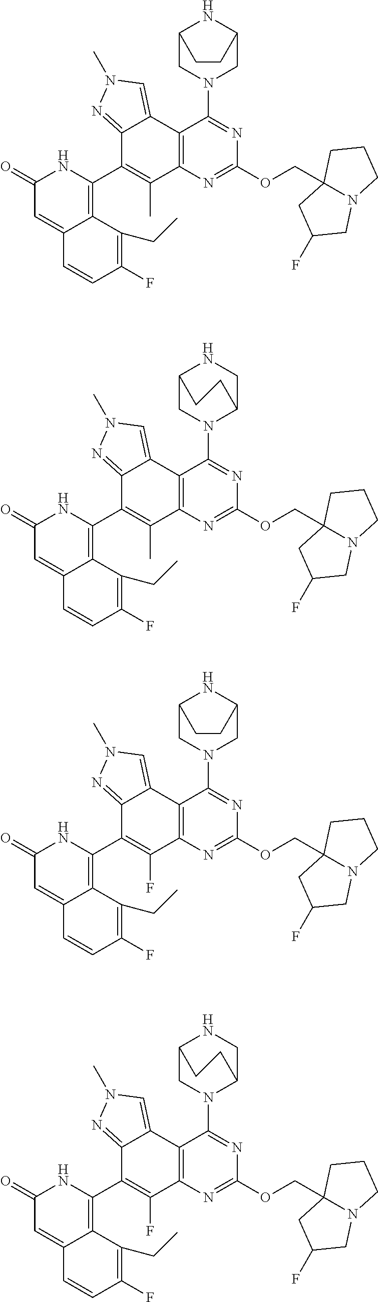 [SMILES: CCc1c(F)ccc2cc(=O)[nH]c(-c3c(C)c4nc(OCC56CCCN5CC(F)C6)nc(N5CC6CCC(C5)N6)c4c4cn(C)nc34)c12 ; CCc1c(F)ccc2cc(=O)[nH]c(-c3c(C)c4nc(OCC56CCCN5CC(F)C6)nc(N5CC6CCC5CN6)c4c4cn(C)nc34)c12 ; CCc1c(F)ccc2cc(=O)[nH]c(-c3c(F)c4nc(OCC56CCCN5CC(F)C6)nc(N5CC6CCC(C5)N6)c4c4cn(C)nc34)c12 ; CCc1c(F)ccc2cc(=O)[nH]c(-c3c(F)c4nc(OCC56CCCN5CC(F)C6)nc(N5CC6CCC5CN6)c4c4cn(C)nc34)c12]

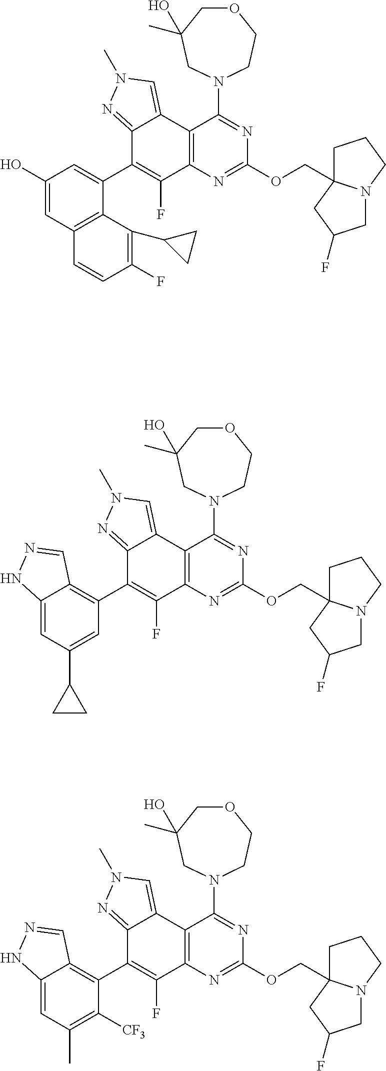 [SMILES: Cn1cc2c(n1)c(-c1cc(O)cc3ccc(F)c(C4CC4)c13)c(F)c1nc(OCC34CCCN3CC(F)C4)nc(N3CCOCC(C)(O)C3)c12 ; Cn1cc2c(n1)c(-c1cc(C3CC3)cc3[nH]ncc13)c(F)c1nc(OCC34CCCN3CC(F)C4)nc(N3CCOCC(C)(O)C3)c12 ; Cc1cc2[nH]ncc2c(-c2c(F)c3nc(OCC45CCCN4CC(F)C5)nc(N4CCOCC(C)(O)C4)c3c3cn(C)nc23)c1C(F)(F)F]

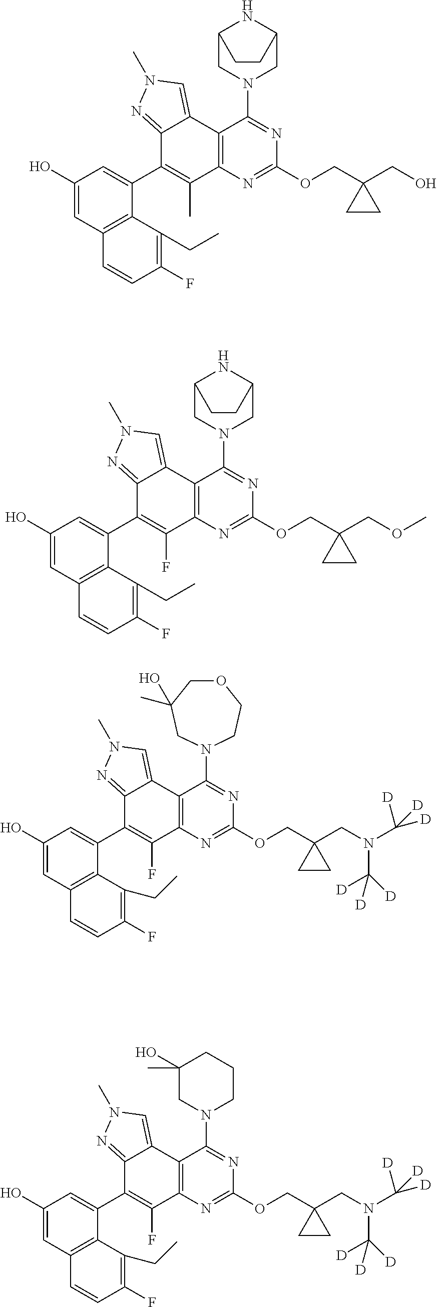 [SMILES: CCc1c(F)ccc2cc(O)cc(-c3c(C)c4nc(OCC5(CO)CC5)nc(N5CC6CCC(C5)N6)c4c4cn(C)nc34)c12 ; CCc1c(F)ccc2cc(O)cc(-c3c(F)c4nc(OCC5(COC)CC5)nc(N5CC6CCC(C5)N6)c4c4cn(C)nc34)c12 ; [2H]C([2H])([2H])N(CC1(COc2nc(N3CCOCC(C)(O)C3)c3c(n2)c(F)c(-c2cc(O)cc4ccc(F)c(CC)c24)c2nn(C)cc23)CC1)C([2H])([2H])[2H] ; [2H]C([2H])([2H])N(CC1(COc2nc(N3CCCC(C)(O)C3)c3c(n2)c(F)c(-c2cc(O)cc4ccc(F)c(CC)c24)c2nn(C)cc23)CC1)C([2H])([2H])[2H]]

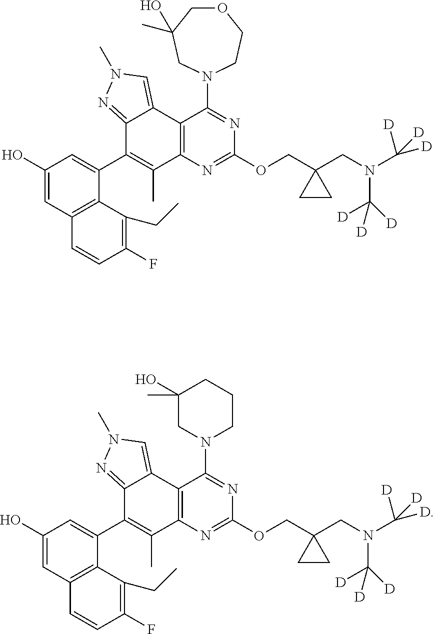

64. A compound of claim 35 for medical use.

65. A compound of claim 35 for use in treating cancer; optionally wherein the cancer is selected from: pancreatic carcinoma, colorectal carcinoma, rectal carcinoma, endometrial carcinoma, non-small cell lung carcinoma, gastric carcinoma, ovarian carcinoma and small cell lung carcinoma.

66. A compound for use of claim 65, wherein the subject being treated has a cancer having wild-type KRAS.

67. A compound for use of claim 66, wherein the subject being treated has a cancer having a KRAS mutation selected from: KRAS G12D, KRAS G12C, KRAS G12V, KRAS G12A, KRAS G12D, KRAS G12S, KRAS G13D and KRAS Q61H.

68. A pharmaceutical composition comprising a compound of claim 35 and a pharmaceutically acceptable excipient.
